(19) United States
(12) Patent Application Publication
(10) Pub. No.: US 2025/0353837 A1
(43) Pub. Date: Nov. 20, 2025

(54) HETEROCYCLIC COMPOUND, ORGANIC LIGHT EMITTING DEVICE AND COMPOSITION FOR ORGANIC MATERIAL LAYER OF ORGANIC LIGHT EMITTING DEVICE

(71) Applicant: LT MATERIALS CO.,LTD., Yongin-si (KR)

(72) Inventors: Seon-Min PARK, Yongin-si (KR); Geon-Yu PARK, Yongin-si (KR); Young-Seok NO, Yongin-si (KR); Dong-Jun KIM, Yongin-si (KR); Dae-Hyuk CHOI, Yongin-si (KR)

(21) Appl. No.: 19/203,184

(22) Filed: May 9, 2025

(30) Foreign Application Priority Data
May 17, 2024 (KR) 10-2024-0064421

(51) Int. Cl. C07D 405/14; C07D 403/10; C07D 405/10; C07D 409/10; C07D 409/14; H10K 85/60

(52) U.S. Cl. CPC C07D 405/14; C07D 403/10; C07D 405/10; C07D 409/10; C07D 409/14; H10K 85/622; H10K 85/654; H10K 85/6572; H10K 85/6574; H10K 85/6576

(57) ABSTRACT

Disclosed area heterocyclic compound of Chemical Formula 1, an organic light emitting device including the same, and a composition for an organic material layer of an organic light emitting device. When the heterocyclic compound is used for an organic light emitting device, the driving voltage of the device can be lowered, the light efficiency of the device can be improved, and the thermal stability of the heterocyclic compound can be improved to improve the service life characteristics of the device.

### CROSS REFERENCE TO RELATED APPLICATIONS

This application claims priority to and the benefit of Korean Patent Application No. 10-2024-0064421 filed in the Korean Intellectual Property Office on May 17, 2024, the entire contents of which are incorporated herein by reference.

### TECHNICAL FIELD

The present specification relates to a heterocyclic compound, an organic light emitting device, and a composition for an organic material layer of an organic light emitting device.

### BACKGROUND ART

A light emitting device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENTS

### Patent Documents

(Patent Document 1) U.S. Pat. No. 4,356,429

### SUMMARY OF THE INVENTION

The present specification has been made in an effort to provide a heterocyclic compound, an organic light emitting device, and a composition for an organic material layer of an organic light emitting device.

An exemplary embodiment of the present specification provides a heterocyclic compound represented by the following Chemical Formula 1.

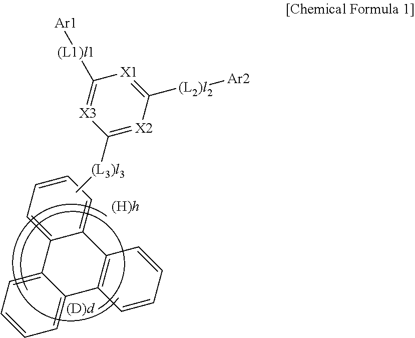

In Chemical Formula 1,
X1 to X3 are the same as or different from each other, and are each N or CRa, and one or more of X1 to X3 are N,
Ra is hydrogen; a substituted or unsubstituted C6 to C60 aryl group; or a substituted or unsubstituted C2 to C60 heteroaryl group,
L1 to L3 are the same as or different from each other, and are each independently a direct bond; a substituted or unsubstituted C6 to C60 arylene group; or a substituted or unsubstituted C2 to C60 heteroarylene group, and at least one of L1 and L2 is a substituted or unsubstituted C6 to C60 arylene group,
Ar1 and Ar2 are the same as or different from each other, and are each independently a substituted or unsubstituted C6 to C60 aryl group; or a substituted or unsubstituted C2 to C60 heteroaryl group,
I1 to I3 are the same as or different from each other, and are each independently an integer from 1 to 4, and when each of I1 to I3 is an integer of 2 or higher, substituents in the parenthesis are the same as or different from each other,
H is hydrogen,
D is deuterium,
h is an integer from 0 to 10,
d is an integer from 1 to 11, and
the sum of h and d is 11.

Another exemplary embodiment of the present specification provides an organic light emitting device including: a first electrode; a second electrode disposed to face the first electrode; and an organic material layer having one or more layers disposed between the first electrode and the second electrode, in which one or more layers of the organic material layer include the above-described heterocyclic compound.

Yet another exemplary embodiment of the present specification provides a composition for forming an organic material layer of an organic light emitting device, including the above-described heterocyclic compound and a heterocyclic compound represented by the following Chemical Formula 2 or 3.

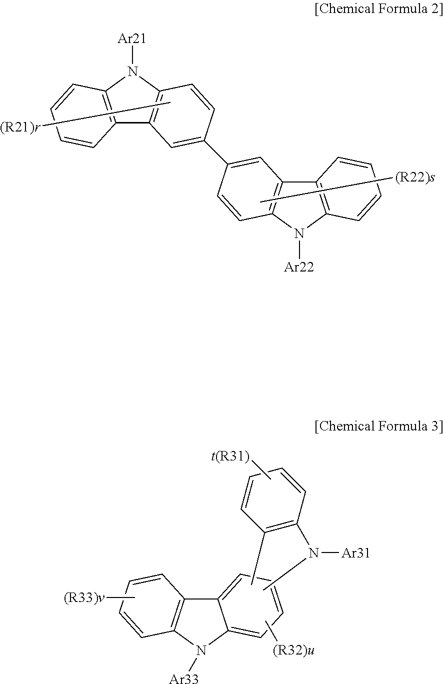

In Chemical Formulae 2 and 3,
R21, R22, and R31 to R33 are the same as or different from each other, and are each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR′R″R′″; —P(═O)R′R″; and an amine group which is unsubstituted or substituted with a substituted or unsubstituted C1 to C60 alkyl group, a substituted or unsubstituted C6 to C60 aryl group or a substituted or unsubstituted C2 to C60 heteroaryl group, or two or more adjacent groups are bonded to each other to form a substituted or unsubstituted C6 to C60 aliphatic or aromatic hydrocarbon ring or a substituted or unsubstituted C2 to C60 aliphatic or aromatic hetero ring,
R′, R″, and R′″ are the same as or different from each other, and are each independently hydrogen; deuterium; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r and s are each an integer from 0 to 7, and when each of r and s is 2 or higher, substituents in the parenthesis are the same as or different from each other,
t and v are each an integer from 0 to 4, and when each of t and v is 2 or higher, substituents in the parenthesis are the same as or different from each other,
u is an integer from 0 to 2, and when u is 2, substituents in the parenthesis are the same as or different from each other, and
Ar21, Ar22, Ar31, and Ar33 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

The heterocyclic compound according to an exemplary embodiment of the present application is an azine 1-substituted triphenylene core represented by Chemical Formula 1, and is characterized by a structure in which at least one of the 11 carbon atoms of triphenylene is linked to deuterium, except for the carbon to which an azine-based substituent is linked. Further, the heterocyclic compound according to an exemplary embodiment of the present application is additionally characterized in that the azine-based substituent includes two substituents in addition to a bond linked to triphenylene, and at least one linker (L1 and/or L2) in a first substituent (-(L1)|1-Ar1) and second substituent (-(L2)|2-Ar2) thereof is a substituted or unsubstituted C6 to C60 arylene group.

As a result, when the heterocyclic compound is used in an organic light emitting device, the glass transition temperature (Tg) is high, so that the driving stability of the device is enhanced because the thermal stability is excellent. In addition, by substituting the heterocyclic compound with deuterium, which has a 2-fold higher atomic mass than hydrogen, the bond dissociation energy between carbon and deuterium can be increased, thereby improving the structural stability of the compound and the service life characteristics of the device.

### BRIEF DESCRIPTION OF THE DRAWINGS

FIGS. 1 to 3 are views each exemplarily illustrating a stacking structure of an organic light emitting device according to an exemplary embodiment of the present specification.

### DETAILED DESCRIPTION

Hereinafter, the present specification will be described in more detail.

### Definitions

When one part “includes” one constituent element in the present specification, unless otherwise specifically described, this does not mean that another constituent element is excluded, but means that another constituent element may be further included.

In the present specification,

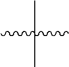

of a chemical formula or structural formula means a position to be bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

Unless particularly defined in the present specification, “substituted or unsubstituted” means to be unsubstituted or substituted with one or more substituents selected from the group consisting of a cyano group; a halogen group; a C1 to C60 straight-chained or branched alkyl group; a C2 to C60 straight-chained or branched alkenyl group; a C2 to C60 straight-chained or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; a silyl group; a phosphine oxide group; and an amine group, or to be unsubstituted or substituted with a substituent to which two or more substituents selected from among the exemplified substituents are linked.

In the present specification, deuterium (D) is one of the isotopes of hydrogen, is an element that has a deuteron composed of one proton and one neutron as a nucleus, and may be represented by hydrogen-2, and the element symbol may also be expressed as D or ²H.

In the present specification, the isotope means an atom with the same atomic number (Z), but different mass numbers (A), and may also be interpreted as an element which has the same number of protons, but different number of neutrons.

According to an exemplary embodiment of the present specification, when the total number of substituents of a basic compound is defined as T1 and the number of specific substituents among the substituents is defined as T2, the content T % of the specific substituent may be defined as T2/T1×100=T %.

That is, when taking a phenyl group represented by

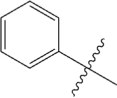 [SMILES: *c1ccccc1]

as an example, herein, a deuterium content of 20% may be represented by 20% when the total number of substituents that the phenyl group can have is 5 (T1 in the formula) and the number of deuterium atoms among the substituents is 1 (T2 in the formula). That is, a deuterium content of 20% in the phenyl group may be represented by the following structural formula.

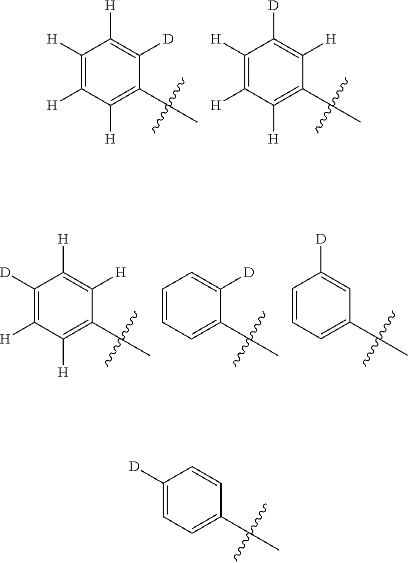

In the present specification, “a phenyl group having a deuterium content of 0%” may mean a phenyl group that does not include a deuterium atom, that is, has five hydrogen atoms.

In the present specification, the cyano group may mean —CN.

In the present specification, the halogen group may be fluorine, chlorine, bromine, or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group; a 2-butenyl group; a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. H ere, the polycycle means a group in which a cycloalkyl group is directly linked to or fused with another cyclic group. H ere, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

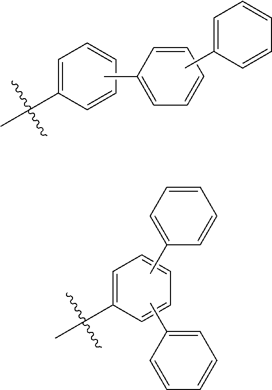

In the present specification, the fluorenyl group may be substituted, and adjacent substituents may be bonded to each other to form a ring.

When the fluorenyl group is substituted, the substituent may be selected from the following structures, but is not limited thereto.

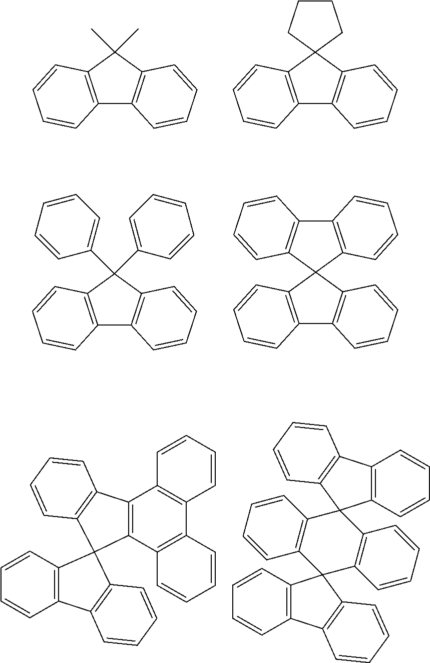

In the present specification, the heteroaryl group includes S, O, Se, N, or Si as a heteroatom, includes a monocycle or a polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 2,3-dihydrobenzo[b]thiophene group, a 2,3-dihydrobenzofuran group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, when the substituent is a carbazole group, it means being bonded to nitrogen or carbon of carbazole.

In the present specification, when a carbazole group is substituted, an additional substituent may be substituted with a nitrogen or carbon of the carbazole, and when the substituents are adjacent, two or more adjacent substituents may be bonded to each other to form a ring. The ring formed herein may be a substituted or unsubstituted benzene ring, a substituted or unsubstituted naphthalene ring, a substituted or unsubstituted indene ring, a substituted or unsubstituted indole ring, a substituted or unsubstituted benzofuran ring, a substituted or unsubstituted benzothiophene ring, or a combination thereof. For example, when an additional substituent is substituted with a carbon of the carbazole and the substituents are adjacent, two or more adjacent substituents are bonded to each other to form a benzene ring, which is equivalent to the meaning of a benzocarbazole group.

In the present specification, a benzocarbazole group may be any one of the following structures.

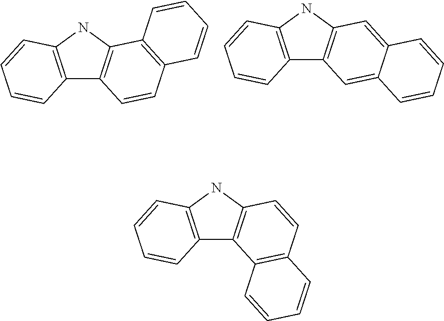

In the present specification, a dibenzocarbazole group may be any one of the following structures.

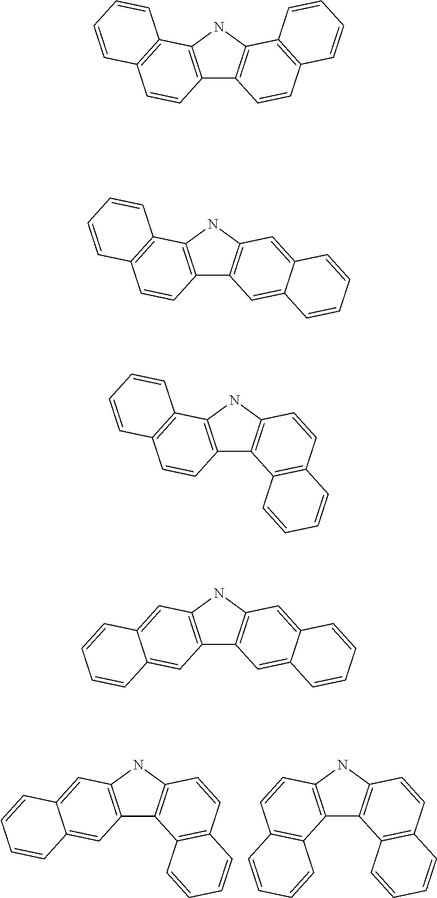

In the present specification, a naphthobenzofuran group may be any one of the following structures.

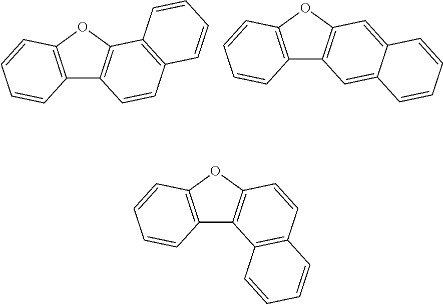

In the present specification, a naphthobenzothiophene group may be any one of the following structures.

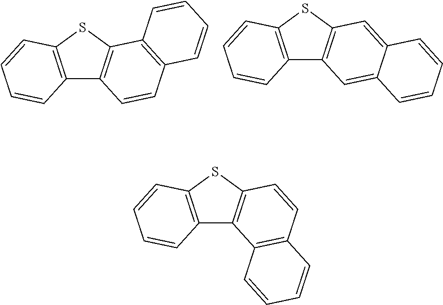

In the present specification, a silyl group includes Si and is a substituent to which the Si atom is directly linked as a radical, and is represented by —Si(R101)(R102)(R103), and R101 to R103 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

Specific examples of the silyl group include

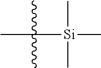 [SMILES: *[Si](C)(C)C]

(a trimethylsilyl group),

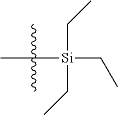 [SMILES: *[Si](CC)(CC)CC]

(a triethylsilyl group),

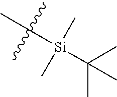 [SMILES: *[Si](C)(C)C(C)(C)C]

(a t-butyldimethylsilyl group),

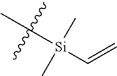 [SMILES: *[Si](C)(C)C=C]

(a vinyldimethylsilyl group),

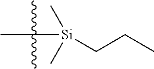 [SMILES: *[Si](C)(C)CCC]

(a propyldimethylsilyl group),

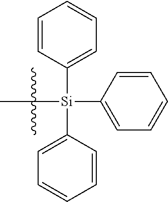 [SMILES: *[Si](c1ccccc1)(c1ccccc1)c1ccccc1]

(a triphenylsilyl group),

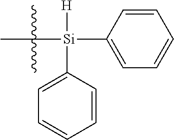 [SMILES: *[SiH](c1ccccc1)c1ccccc1]

(a diphenylsilyl group),

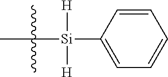 [SMILES: *[SiH2]c1ccccc1]

(a phenylsilyl group) and the like, but are not limited thereto.

In the present specification, the phosphine oxide group is represented by —P(═O)(R104)(R105), and R104 and R105 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, a dinaphthylphosphine oxide group, and the like, but are not limited thereto.

In the present specification, the amine group is represented by —N(R106)(R107), and R106 and R107 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be each applied to the rings, except for those that are not monovalent groups.

### <Heterocyclic Compound>

Hereinafter, the heterocyclic compound according to the present specification will be described.

The heterocyclic compound according to an exemplary embodiment of the present specification may be represented by the following Chemical Formula 1.

### [Chemical Formula 1]

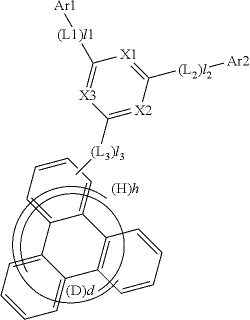

In Chemical Formula 1, the description of each substituent is the same as that described above.

The heterocyclic compound represented by Chemical Formula 1 is an azine-substituted triphenylene, and is characterized in that all 11 carbon atoms of the triphenylene except for the azine-substituted carbon are linked to deuterium, and at least one of L1 and L2 of the azine substituent is a substituted or unsubstituted C6 to C60 arylene group.

As shown in Chemical Formula 1, azine-based substituents are 1-substituted on triphenylene, but when a heterocyclic compound in which only a triphenylene moiety is substituted with deuterium is used in an organic light emitting device, the glass transition temperature (Tg) is high, so that the driving stability of the device may be enhanced because thermal stability is excellent. In addition, by substituting the heterocyclic compound with deuterium, which has a 2-fold higher atomic mass than hydrogen, the bond dissociation energy between carbon and deuterium can be increased, thereby improving the structural stability of the compound and the service life characteristics of the device.

The heterocyclic compound may be characterized by being a structure in which only the triphenylene moiety is substituted with deuterium to strengthen the LUMO and an arylene linker is introduced into the end of the azine-based substituent, thereby reducing the degree of overlap between the HOMO and LUMO orbitals, and as a result, the stability of electrons and holes may be improved.

According to an exemplary embodiment of the present specification, Chemical Formula 1 may be represented by the following Chemical Formula 1-1 or 1-2.

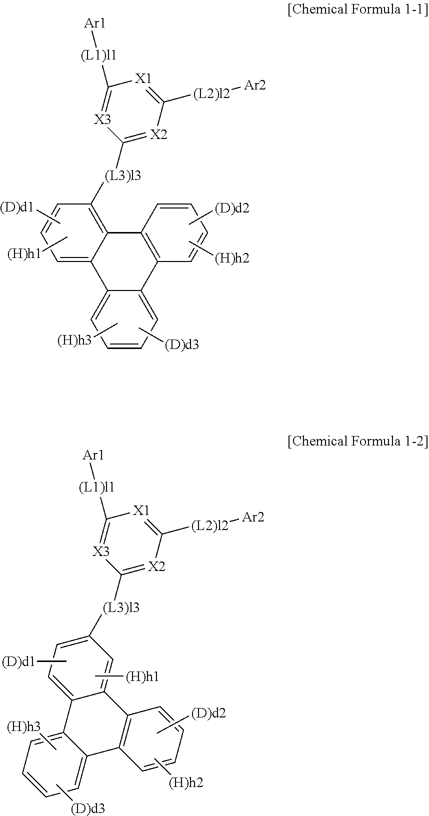

In Chemical Formulae 1-1 and 1-2,
each of X1 to X3, L1 to L3, Ar1, Ar2, I1 to I3, H, and D is the same as that defined in Chemical Formula 1,
h1 is an integer from 0 to 3,
h2 and h3 are each an integer from 0 to 4,
d1 is an integer from 0 to 3,
d2 and d3 are each an integer from 0 to 4,
the sum of h1 and d1 is 3,
the sum of h2 and d2 and the sum of h3 and d3 are each 4, and
the sum of d1, d2, and d3 are an integer from 1 to 11.

According to an exemplary embodiment of the present specification, “substituted or unsubstituted” means being unsubstituted or substituted with one or more substituents selected from the group consisting of a C1 to C60 alkyl group; a C3 to C60 cycloalkyl group; a C2 to C60 heterocycloalkyl group; a C6 to C60 aryl group; a C2 to C60 heteroaryl group; —SiR′R″R′″; and —P(═O)R′R″, or being unsubstituted or substituted with a substituent to which two or more substituents selected from the above substituents are linked, or means that two or more substituents selected from the above substituents are bonded to each other to form a ring, and
R′, R″, and R′″ are the same as or different from each other, and are each independently hydrogen; a C1 to C60 alkyl group; a C3 to C60 cycloalkyl group; a C6 to C60 aryl group; or a C2 to C60 heteroaryl group.

According to an exemplary embodiment of the present specification, the deuterium content of

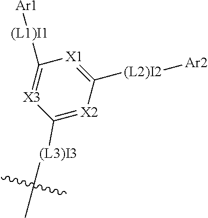

### of Chemical Formula 1 is 0%, and

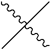

means a linking position in Chemical Formula 1.

The heterocyclic compound according to the exemplary embodiments is characterized in that only a limited region is substituted with deuterium, meaning that the remaining 11 carbon atoms of triphenylene, except for the carbon linked to the azine-based substituent, are linked to deuterium, and when the heterocyclic compound having such characteristics is used in an organic light emitting device in the future, the organic light emitting device may have low driving voltage, high light emitting efficiency, and/or long service life characteristics.

According to an exemplary embodiment of the present specification, Ra may be hydrogen; a substituted or unsubstituted C6 to C40 aryl group; or a substituted or unsubstituted C2 to C40 heteroaryl group.

According to an exemplary embodiment of the present specification, Ra may be hydrogen; a substituted or unsubstituted C6 to C20 aryl group; or a substituted or unsubstituted C2 to C20 heteroaryl group.

According to an exemplary embodiment of the present specification, X1 to X3 are the same or different from each other, and are each N or CRa, and two or more groups of X1 to X3 may be N.

According to an exemplary embodiment of the present specification, all of X1 to X3 may be N.

According to an exemplary embodiment of the present specification, L1 to L3 are the same as or different from each other, and are each independently a direct bond; a substituted or unsubstituted C6 to C40 arylene group; or a substituted or unsubstituted C2 to C40 heteroarylene group, and at least one of L1 and L2 may be a substituted or unsubstituted C6 to C40 arylene group.

According to an exemplary embodiment of the present specification, L1 to L3 are the same as or different from each other, and are each independently a direct bond; a substituted or unsubstituted C6 to C30 arylene group; or a substituted or unsubstituted C2 to C30 heteroarylene group, and at least one of L1 and L2 may be a substituted or unsubstituted C6 to C30 arylene group.

According to an exemplary embodiment of the present specification, L1 to L3 are the same as or different from each other, and are each independently a direct bond; or a substituted or unsubstituted C6 to C30 arylene group, and at least one of L1 and L2 may be a substituted or unsubstituted C6 to C30 arylene group.

According to an exemplary embodiment of the present specification, L1 to L3 are the same as or different from each other, and are each independently a direct bond; or an unsubstituted C6 to C30 arylene group, and at least one of L1 and L2 may bean unsubstituted C6 to C30 arylene group.

According to an exemplary embodiment of the present specification, L1 to L3 are the same as or different from each other, and are each independently a direct bond; or a substituted or unsubstituted phenylene, and at least one of L1 and L2 may be a substituted or unsubstituted phenylene group.

According to an exemplary embodiment of the present specification, L1 to L3 are the same as or different from each other, and are each independently a direct bond; or a phenylene group, and at least one of L1 and L 2 may be a phenylene group.

According to an exemplary embodiment of the present specification, Ar1 and Ar2 are the same as or different from each other, and may be each independently a substituted or unsubstituted C6 to C30 aryl group; or a substituted or unsubstituted C2 to C30 heteroaryl group.

According to an exemplary embodiment of the present specification, Ar1 and Ar2 are the same as or different from each other, and may be each independently a C6 to C30 aryl group unsubstituted or substituted with an aryl group; or a C2 to C30 heteroaryl group unsubstituted or substituted with an aryl group.

According to an exemplary embodiment of the present specification, Ar1 and Ar2 are the same as or different from each other, and may be each independently a phenyl group; a biphenyl group; a dibenzofuran group; a dibenzothiophene group; a carbazole group unsubstituted or substituted with phenyl; a carbazole group unsubstituted or substituted with phenyl and fused with an indoline group; a carbazole group unsubstituted or substituted with phenyl and fused with benzofuran; or a carbazole group unsubstituted or substituted with phenyl and fused with benzothiophene.

The heterocyclic compound according to the exemplary embodiments is characterized in that one of the linkers of the azine substituent is not a direct bond as defined above, and when the heterocyclic compound having such characteristics is used in an organic light emitting device in the future, the organic light emitting device may have low driving voltage, high light emitting efficiency, and/or long service life characteristics.

According to an exemplary embodiment of the present specification, h may be an integer from 1 to 10, an integer from 2 to 10, an integer from 3 to 10, an integer from 4 to 10, an integer from 5 to 10, an integer from 6 to 10, an integer from 7 to 10, an integer from 8 to 10, an integer from 9 to 10, or 10.

According to an exemplary embodiment of the present specification, d may be an integer from 3 to 11, an integer from 4 to 11, an integer from 5 to 11, an integer from 6 to 11, an integer from 7 to 11, an integer from 8 to 11, an integer from 9 to 11, 10, or 11.

According to an exemplary embodiment of the present specification, the deuterium content of the heterocyclic compound represented by Chemical Formula 1 may be more than 0% and 41% or less.

According to preferred exemplary embodiments of the present specification, d may be 3 or more. In other words, the deuterium substitution rate of the triphenylene moiety

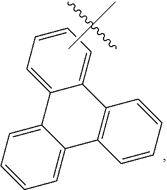

except for carbon linked to a linking moiety

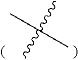

of Chemical Formula 1 may be 27% or more.

According to preferred exemplary embodiments of the present specification, d may be 4 or more. In other words, the deuterium substitution rate of the triphenylene moiety (except for the linking moiety) of Chemical Formula 1 may be 36% or more.

According to preferred exemplary embodiments of the present specification, d may be 5 or more. In other words, the deuterium substitution rate of the triphenylene moiety (except for the linking moiety) of Chemical Formula 1 may be 45% or more.

According to preferred exemplary embodiments of the present specification, d may be 6 or more. In other words, the deuterium substitution rate of the triphenylene moiety (except for the linking moiety) of Chemical Formula 1 may be 54% or more.

According to preferred exemplary embodiments of the present specification, d may be 7 or more. In other words, the deuterium substitution rate of the triphenylene moiety (except for the linking moiety) of Chemical Formula 1 may be 63% or more.

According to preferred exemplary embodiments of the present specification, d may be 8 or more. In other words, the deuterium substitution rate of the triphenylene moiety (except for the linking moiety) of Chemical Formula 1 may be 72% or more.

According to preferred exemplary embodiments of the present specification, d may be 9 or more. In other words, the deuterium substitution rate of the triphenylene moiety (except for the linking moiety) of Chemical Formula 1 may be 81% or more.

According to preferred exemplary embodiments of the present specification, d may be 10 or more. In other words, the deuterium substitution rate of the triphenylene moiety (except for the linking moiety) of Chemical Formula 1 may be 90% or more.

According to preferred exemplary embodiments of the present specification, d may be 11. In other words, the deuterium substitution rate of the triphenylene moiety (except for the linking moiety) of Chemical Formula 1 may be 100%.

According to an exemplary embodiment of the present specification, when L1 is a substituted or unsubstituted C6 to C60 arylene group, Ar1 is a substituted or unsubstituted C2 to C60 heteroaryl group, or when L2 is a substituted or unsubstituted C6 to C60 arylene group, Ar2 may be a substituted or unsubstituted C2 to C60 heteroaryl group.

According to an exemplary embodiment of the present specification, when L1 is a substituted or unsubstituted C6 to C30 arylene group, Ar1 is a substituted or unsubstituted C2 to C30 heteroaryl group, or when L2 is a substituted or unsubstituted C6 to C30 arylene group, Ar2 may be a substituted or unsubstituted C2 to C30 heteroaryl group.

According to an exemplary embodiment of the present specification, when L1 is a substituted or unsubstituted C6 to C30 arylene group, Ar1 is a substituted or unsubstituted C2 to C30 heteroaryl group, or when L2 is a substituted or unsubstituted C6 to C30 arylene group, Ar2 is a substituted or unsubstituted C2 to C30 heteroaryl group, and the heteroatom of the substituted or unsubstituted C2 to C30 heteroaryl group may include at least one of N, O, and S.

According to an exemplary embodiment of the present specification, when L1 is an unsubstituted C6 to C30 arylene group, Ar1 is a substituted or unsubstituted C2 to C30 heteroaryl group, or when L2 is an unsubstituted C6 to C30 arylene group, Ar2 is a substituted or unsubstituted C2 to C30 heteroaryl group, and the heteroatom of the substituted or unsubstituted C2 to C30 heteroaryl group may include at least one of N, O, and S.

According to an exemplary embodiment of the present specification, when L1 is a phenylene group, Ar1 is a substituted or unsubstituted C2 to C30 heteroaryl group, or when L2 is a phenylene group, Ar2 is a substituted or unsubstituted C2 to C30 heteroaryl group, and the heteroatom of the substituted or unsubstituted C2 to C30 heteroaryl group may include at least one of N, O, and S.

According to an exemplary embodiment of the present specification, when L1 is a phenylene group, A r is a substituted or unsubstituted carbazolyl group; a substituted or unsubstituted dibenzofuran group; or a substituted or unsubstituted dibenzothiophene group, or when L2 is a phenylene group, Ar2 may be a substituted or unsubstituted carbazolyl group; a substituted or unsubstituted dibenzofuran group; or a substituted or unsubstituted dibenzothiophene group. Here, the “substituted or unsubstituted” means being unsubstituted or substituted with one or more substituents selected from the group consisting of a C1 to C60 alkyl group; a C3 to C60 cycloalkyl group; a C2 to C60 heterocycloalkyl group; a C6 to C60 aryl group; and a C2 to C60 heteroaryl group, or being unsubstituted or substituted with a substituent to which two or more substituents selected from the above substituents are linked, or means that two or more substituents selected from the above substituents are bonded to each other to form a hydrocarbon ring or hetero ring.

In an exemplary embodiment, at least one of -(L1)|1-Ar1 and -(L2)|2-Ar2 of Chemical Formula 1 may be represented by the following Structural Formula A-1 or A-2.

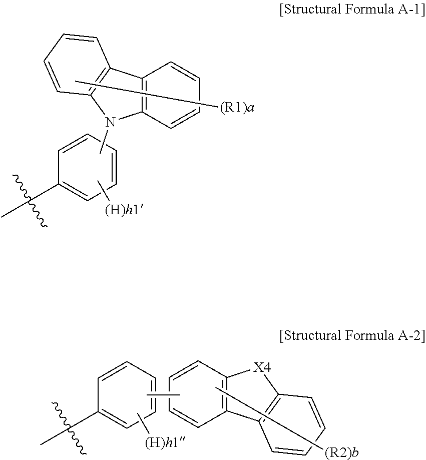

In Structural Formulae A-1 and A-2,

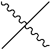

means a linking position in Chemical Formula 1,
H is hydrogen,
h1′ and h1″ are each 4,
X4 is O; S; or NR3,
R1 to R3 are the same as or different from each other, and are each independently hydrogen; an unsubstituted C6 to C60 aryl group; or an unsubstituted C2 to C60 heteroaryl group, or two or more groups are bonded to each other to form a ring unsubstituted or substituted with an aryl group,
a is an integer from 0 to 8, and when a is 2 or higher, R1's in the parenthesis are the same as or different from each other, and
b is an integer from 0 to 7, and when b is 2 or higher, R2's in the parenthesis are the same as or different from each other.

In an exemplary embodiment, R1 and R2 are the same as or different from each other, and are each independently hydrogen; an unsubstituted C6 to C30 aryl group; or an unsubstituted C2 to C30 heteroaryl group, or two or more groups may be bonded to each other to form a C2 to C30 hetero ring unsubstituted or substituted with an aryl group.

In an exemplary embodiment, R1 and R2 are the same as or different from each other, and are each independently hydrogen; or a phenyl group, or two or more groups may be bonded to each other to form an indoline group unsubstituted or substituted with a phenyl group, a benzofuran group unsubstituted or substituted with a phenyl group, or a benzothiophene group unsubstituted or substituted with a phenyl group.

In an exemplary embodiment, any one group of -(L1)|1-Ar1 and -(L2)|2-Ar2 of Chemical Formula 1 may be represented by Structural Formula A-1 or A-2, and the other group may be an unsubstituted C6 to C60 aryl group; or an unsubstituted C2 to C60 heteroaryl group.

In an exemplary embodiment, any one group of -(L1)|1-Ar1 and -(L2)|2-Ar2 of Chemical Formula 1 may be represented by Structural Formula A-1 or A-2, and the other group may be an unsubstituted C6 to C30 aryl group; or an unsubstituted C2 to C30 heteroaryl group, and the heteroatom of the unsubstituted C2 to C30 heteroaryl group may include at least one of O and S.

In an exemplary embodiment, any one group of -(L1)|1-Ar1 and -(L2)|2-Ar2 of Chemical Formula 1 may be represented by Structural Formula A-1 or A-2, and the other group may a phenyl group; a biphenyl group; a dibenzofuran group; or a dibenzothiophene group.

In an exemplary embodiment, Structural Formula A-1 may be represented by any one of the following Structural Formulae A-11 to A-13.

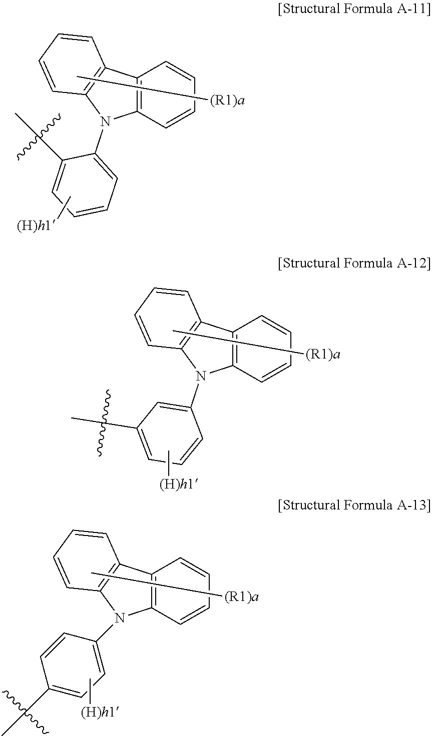

In Structural Formulae A-11 to A-13,

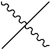

means a linking position in Chemical Formula 1, and
the definitions of H, h1′, R1, and a are the same as those defined in Structural Formula A-1.

In an exemplary embodiment, Structural Formula A-2 may be represented by any one of the following Structural Formulae A-21 to A-32.

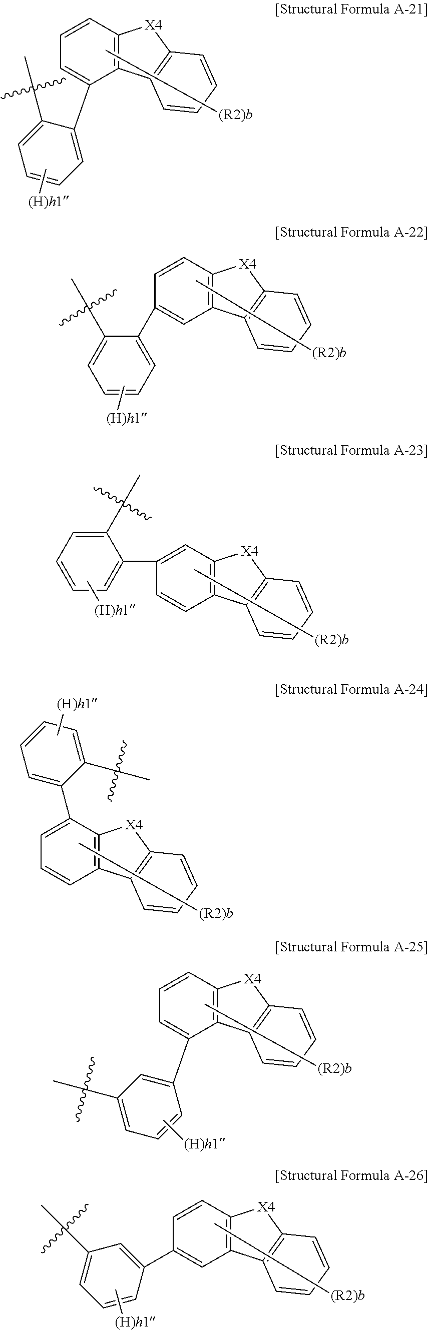

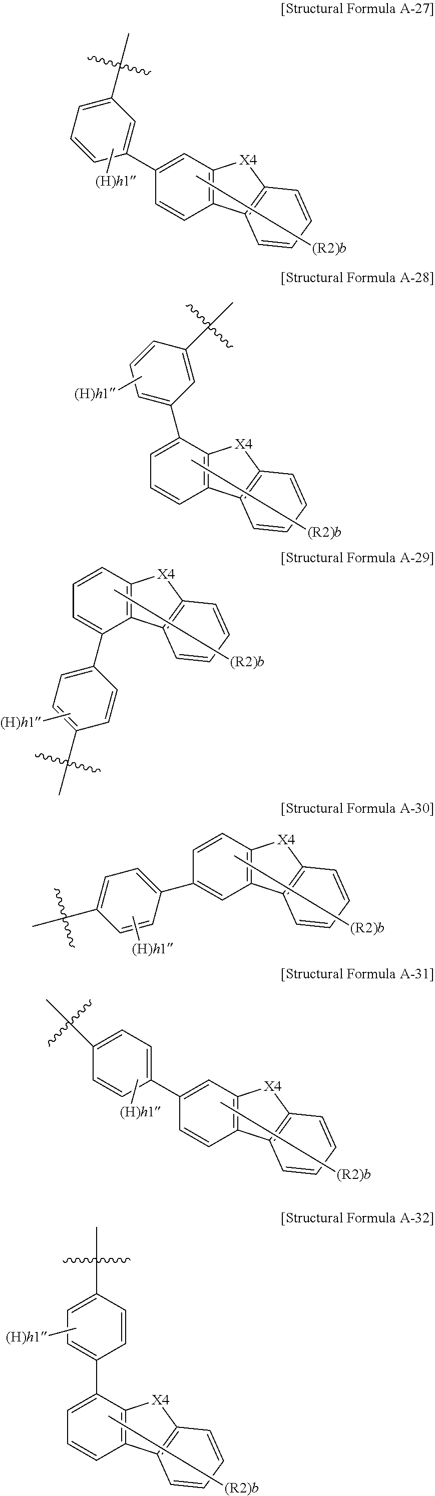

In Structural Formulae A-21 to A-32,

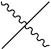

means a linking position in Chemical Formula 1, and
H, h1″, X4, R1, R2, and b are the same as those defined in Structural Formula A-2.

The heterocyclic compound according to the exemplary embodiments is characterized in that at least one (L1 or 12) of the linkers of the azine substituent corresponds to an arylene group such as a phenylene group as defined above, and a terminal group (Ar1 or Ar2) linked thereto is a heteroaryl group such as Structural Formulae A-1, A-11 to A-13, A-2, and A-21 to A-32, and when the heterocyclic group having the characteristics as described above is used in an organic light emitting device in the future, the organic light emitting device may have low driving voltage, high light emitting efficiency, and/or long service life characteristics.

According to an exemplary embodiment of the present specification, L3 may be a direct bond; or a substituted or unsubstituted C6 to C40 arylene group.

According to an exemplary embodiment of the present specification, L3 may be a direct bond; or a substituted or unsubstituted C6 to C30 arylene group.

According to an exemplary embodiment of the present specification, L3 may be a direct bond; or an unsubstituted C6 to C30 arylene group.

According to an exemplary embodiment of the present specification, L3 may be a direct bond; or a phenylene group.

According to an exemplary embodiment of the present specification, L3 may be a direct bond.

According to an exemplary embodiment of the present specification, the heterocyclic compound represented by Chemical Formula 1 may be represented by any one of the followings.

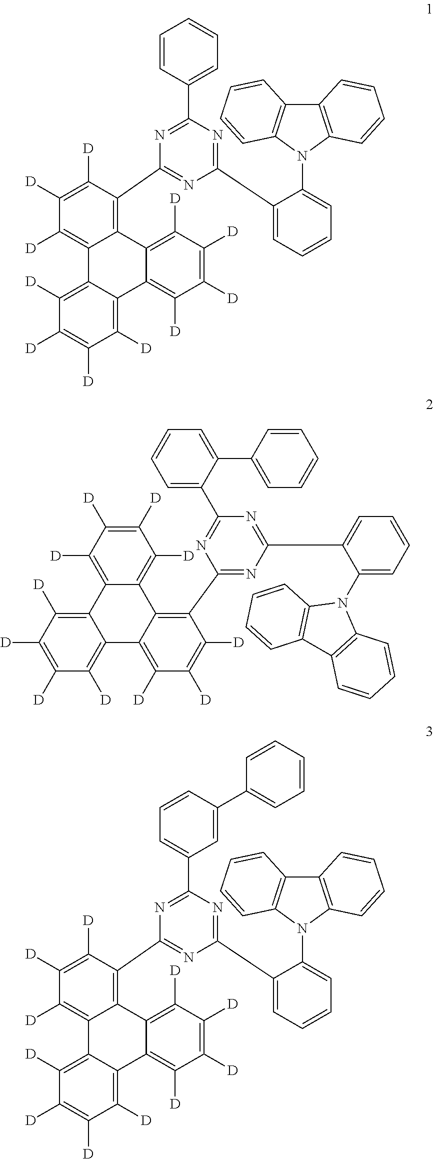 [SMILES: [2H]c1c([2H])c([2H])c2c(c1[2H])c1c([2H])c([2H])c([2H])c([2H])c1c1c(-c3nc(-c4ccccc4)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4-c4ccccc4)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-c5ccccc5)c4)nc(-c4ccccc4-n4c5ccccc5c5ccccc54)n3)c([2H])c([2H])c([2H])c21]

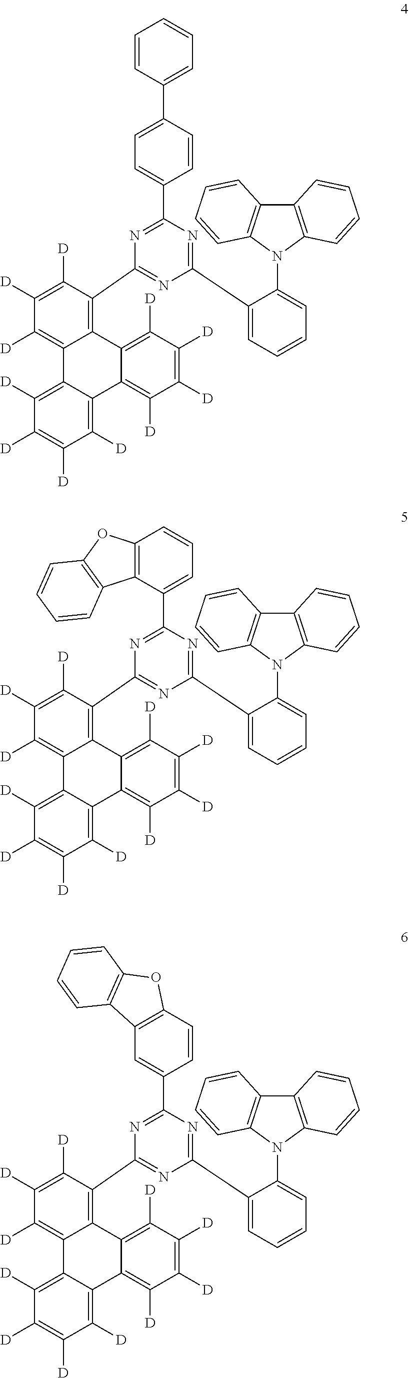 [SMILES: [2H]c1c([2H])c([2H])c2c(c1[2H])c1c([2H])c([2H])c([2H])c([2H])c1c1c(-c3nc(-c4ccc(-c5ccccc5)cc4)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5oc6ccccc6c45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc5oc6ccccc6c5c4)nc(-c4ccccc4-n4c5ccccc5c5ccccc54)n3)c([2H])c([2H])c([2H])c21]

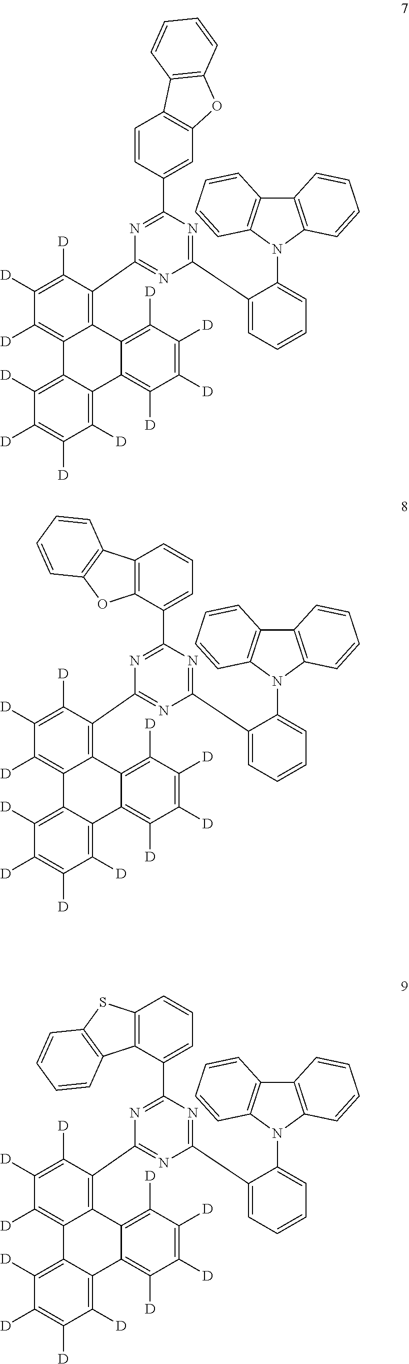

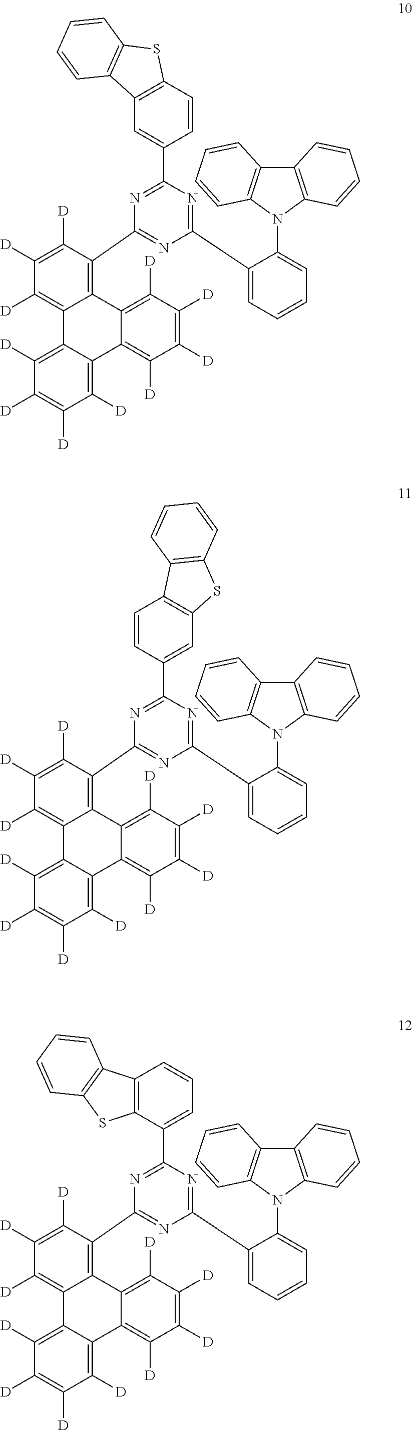 [SMILES: [2H]c1c([2H])c([2H])c2c(c1[2H])c1c([2H])c([2H])c([2H])c([2H])c1c1c(-c3nc(-c4ccc5sc6ccccc6c5c4)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc5c(c4)sc4ccccc45)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5c4sc4ccccc45)n3)c([2H])c([2H])c([2H])c21]

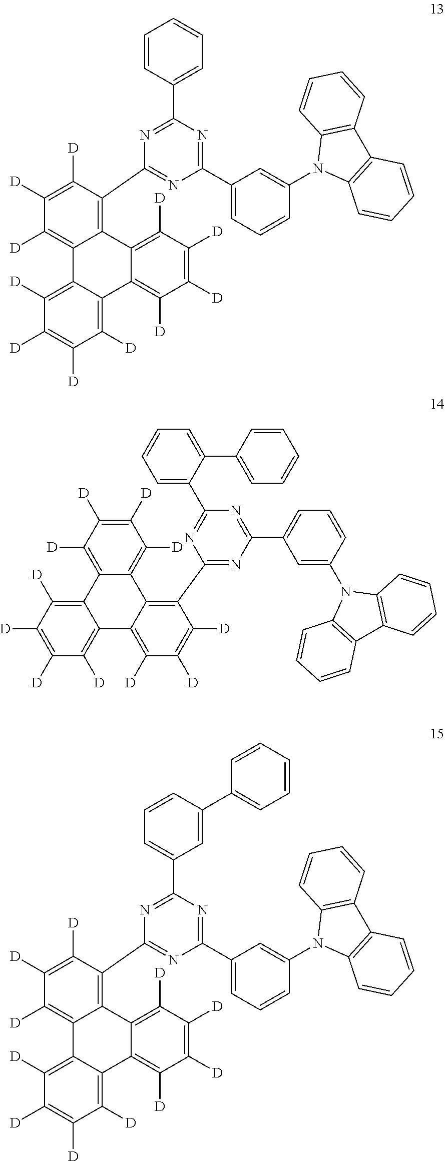 [SMILES: [2H]c1c([2H])c([2H])c2c(c1[2H])c1c([2H])c([2H])c([2H])c([2H])c1c1c(-c3nc(-c4ccccc4)nc(-c4cccc(-n5c6ccccc6c6ccccc65)c4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccccc4-c4ccccc4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-c5ccccc5)c4)nc(-c4cccc(-n5c6ccccc6c6ccccc65)c4)n3)c([2H])c([2H])c([2H])c21]

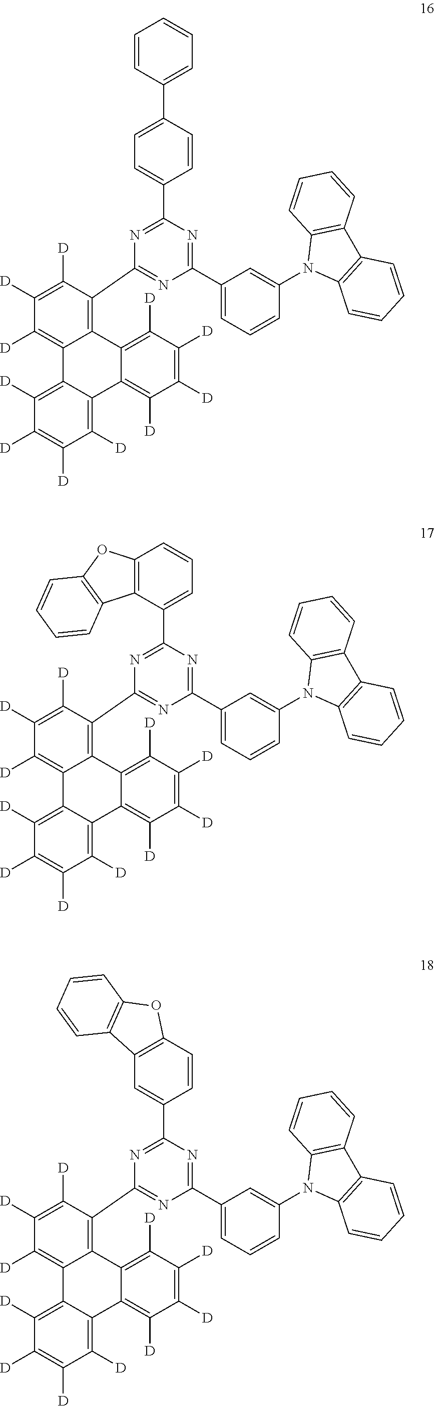 [SMILES: [2H]c1c([2H])c([2H])c2c(c1[2H])c1c([2H])c([2H])c([2H])c([2H])c1c1c(-c3nc(-c4ccc(-c5ccccc5)cc4)nc(-c4cccc(-n5c6ccccc6c6ccccc65)c4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5oc6ccccc6c45)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5oc6ccccc6c5c4)n3)c([2H])c([2H])c([2H])c21]

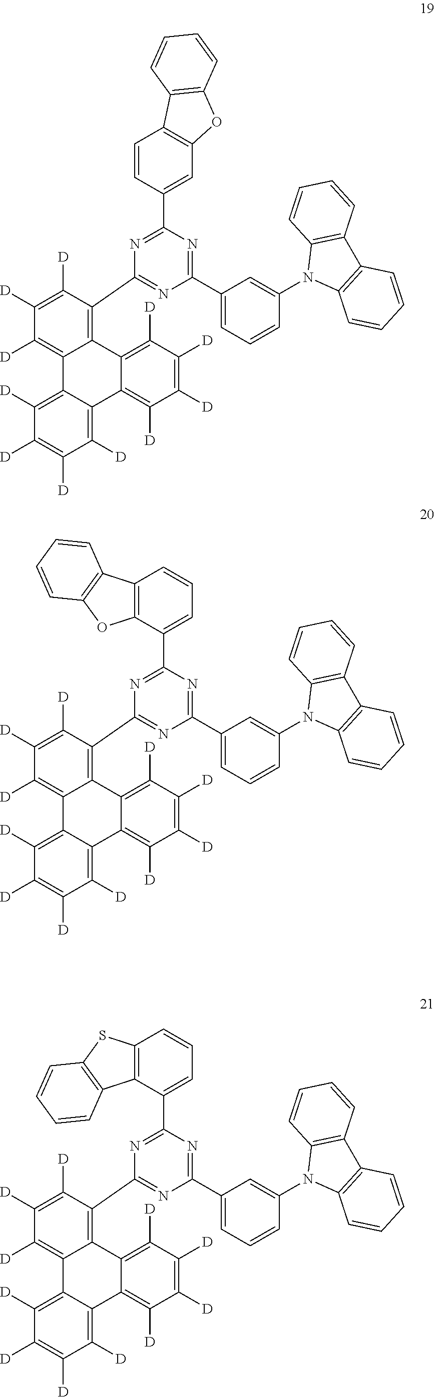 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5c4oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5sc6ccccc6c45)n3)c([2H])c([2H])c([2H])c21]

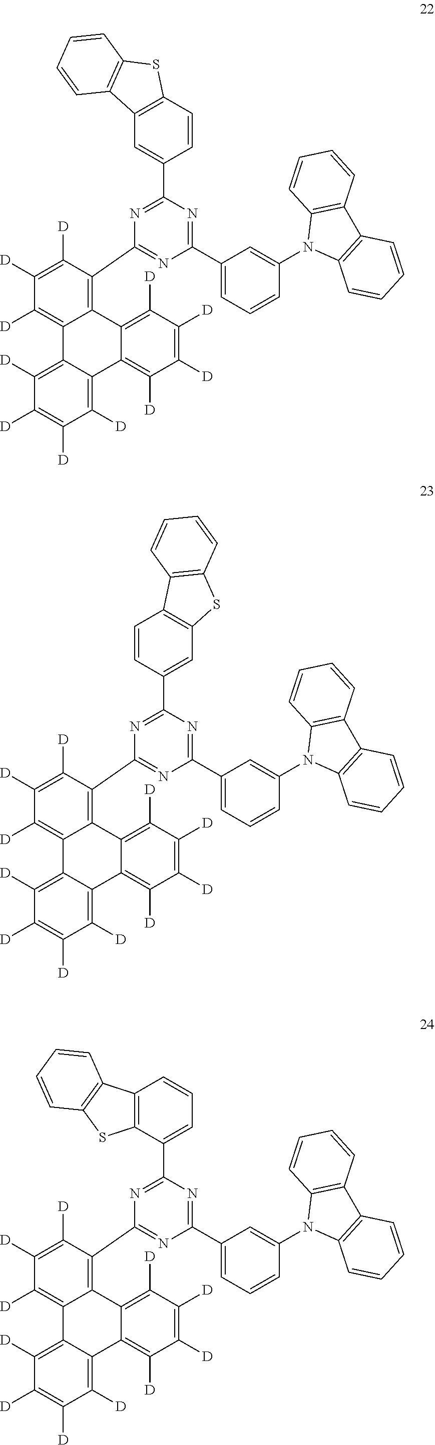 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5sc6ccccc6c5c4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5c(c4)sc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5c4sc4ccccc45)n3)c([2H])c([2H])c([2H])c21]

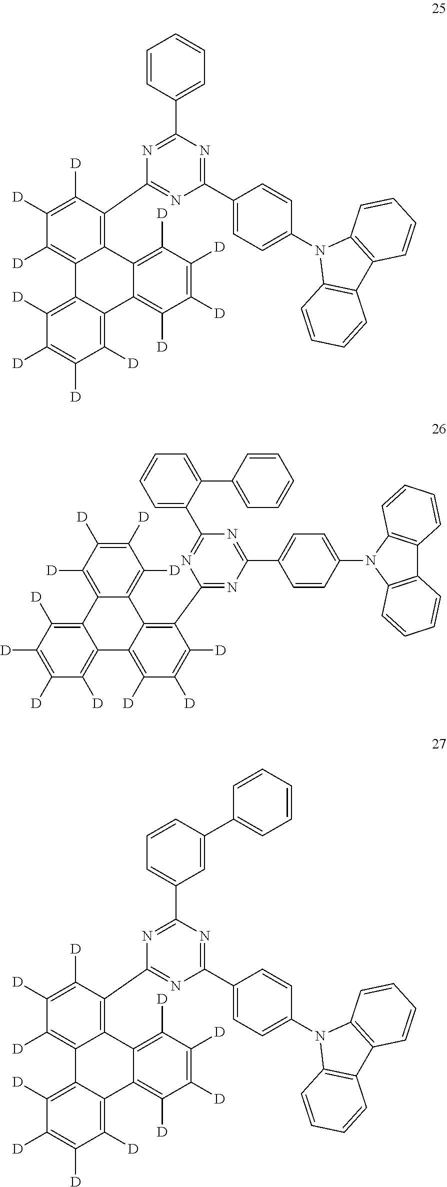 [SMILES: [2H]c1c([2H])c([2H])c2c(c1[2H])c1c([2H])c([2H])c([2H])c([2H])c1c1c(-c3nc(-c4ccccc4)nc(-c4ccc(-n5c6ccccc6c6ccccc65)cc4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccccc4-c4ccccc4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc(-c5ccccc5)c4)n3)c([2H])c([2H])c([2H])c21]

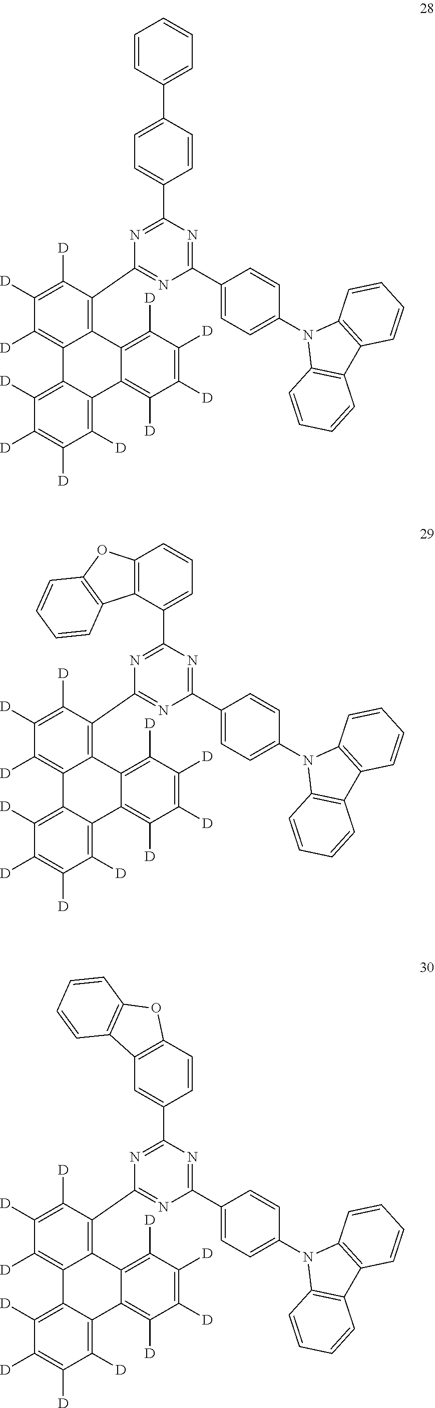 [SMILES: [2H]c1c([2H])c([2H])c2c(c1[2H])c1c([2H])c([2H])c([2H])c([2H])c1c1c(-c3nc(-c4ccc(-c5ccccc5)cc4)nc(-c4ccc(-n5c6ccccc6c6ccccc65)cc4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5oc6ccccc6c45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5oc6ccccc6c5c4)n3)c([2H])c([2H])c([2H])c21]

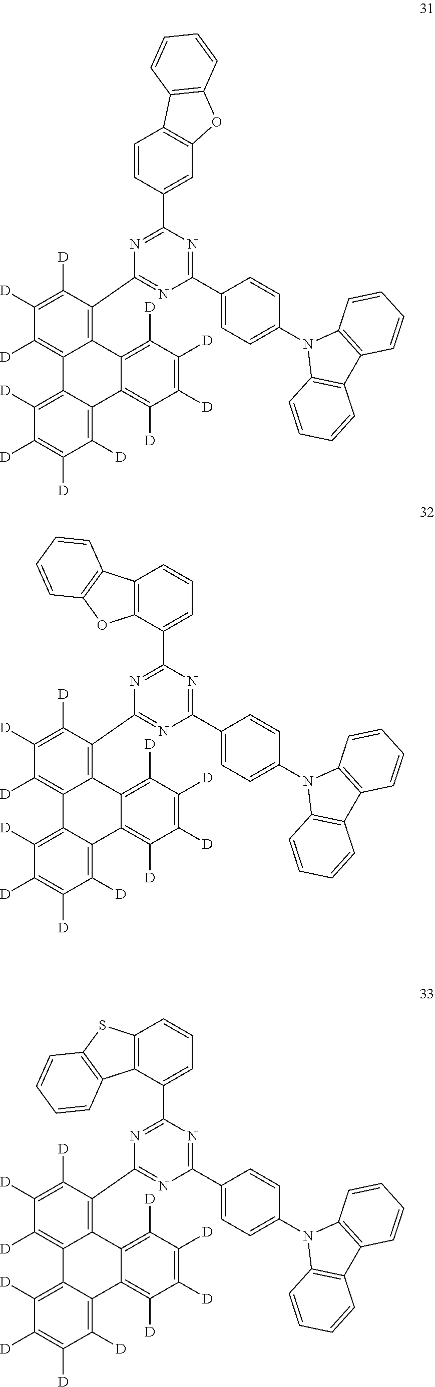 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5c4oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5sc6ccccc6c45)n3)c([2H])c([2H])c([2H])c21]

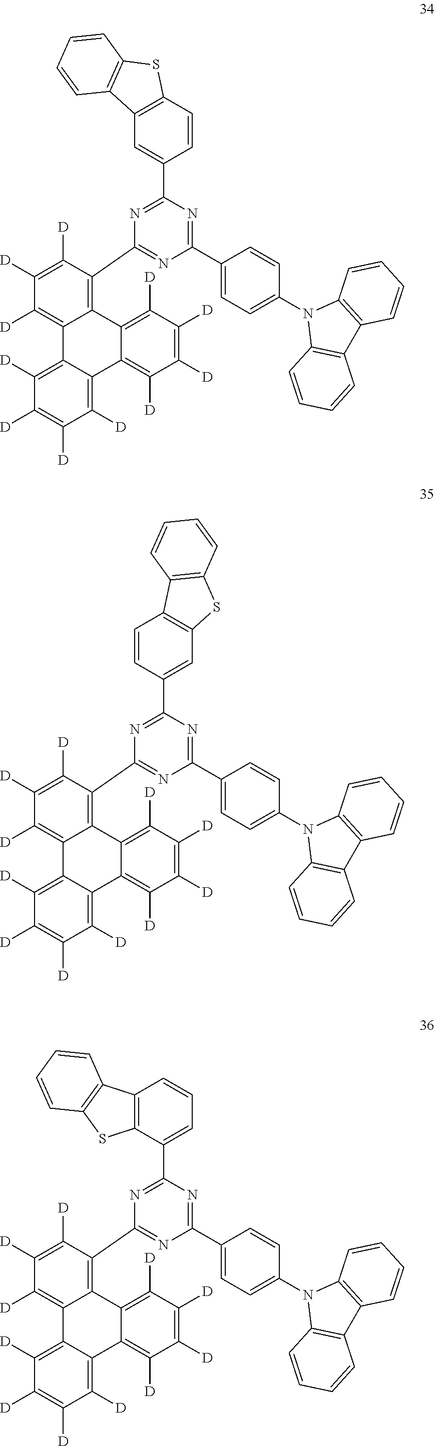 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5sc6ccccc6c5c4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5c(c4)sc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5c4sc4ccccc45)n3)c([2H])c([2H])c([2H])c21]

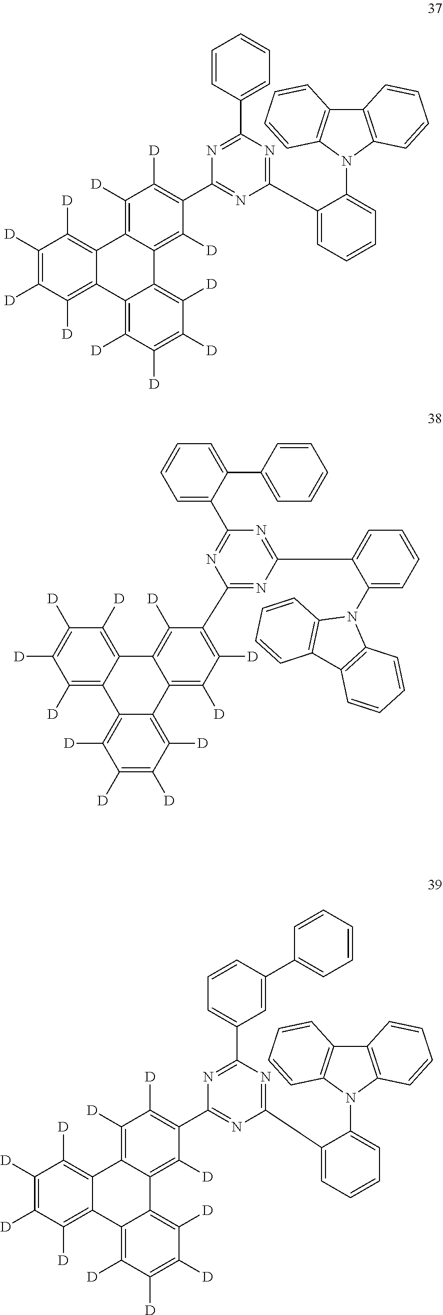 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-n4c5ccccc5c5ccccc54)n3)c([2H])c([2H])c21]

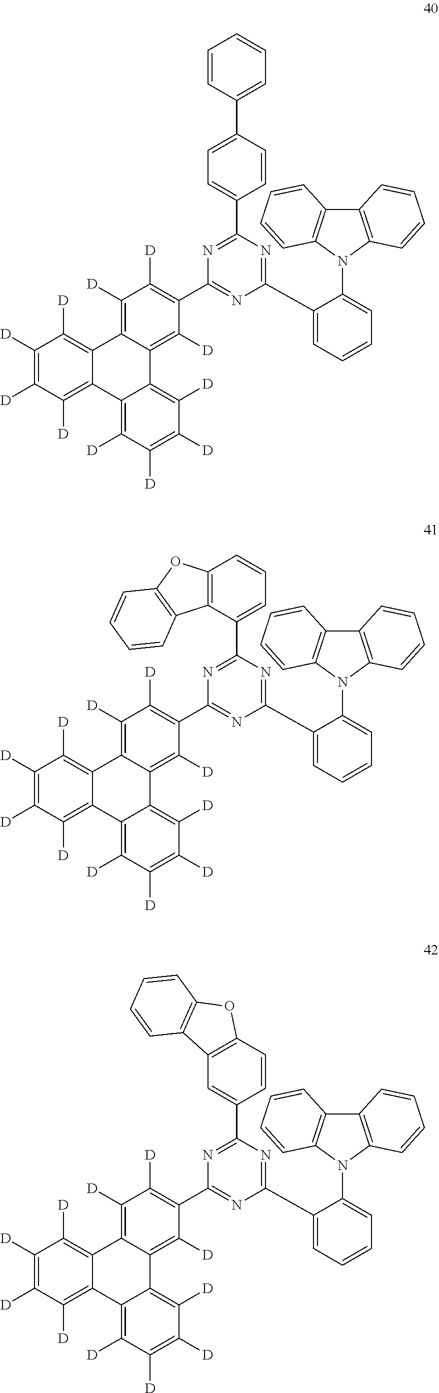 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-n4c5ccccc5c5ccccc54)n3)c([2H])c([2H])c21]

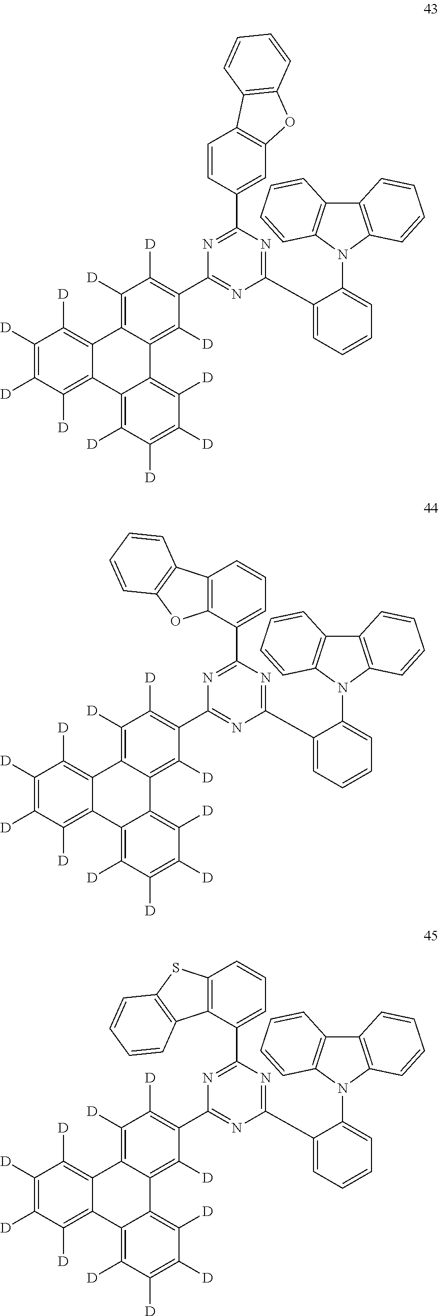 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5sc6ccccc6c45)n3)c([2H])c([2H])c21]

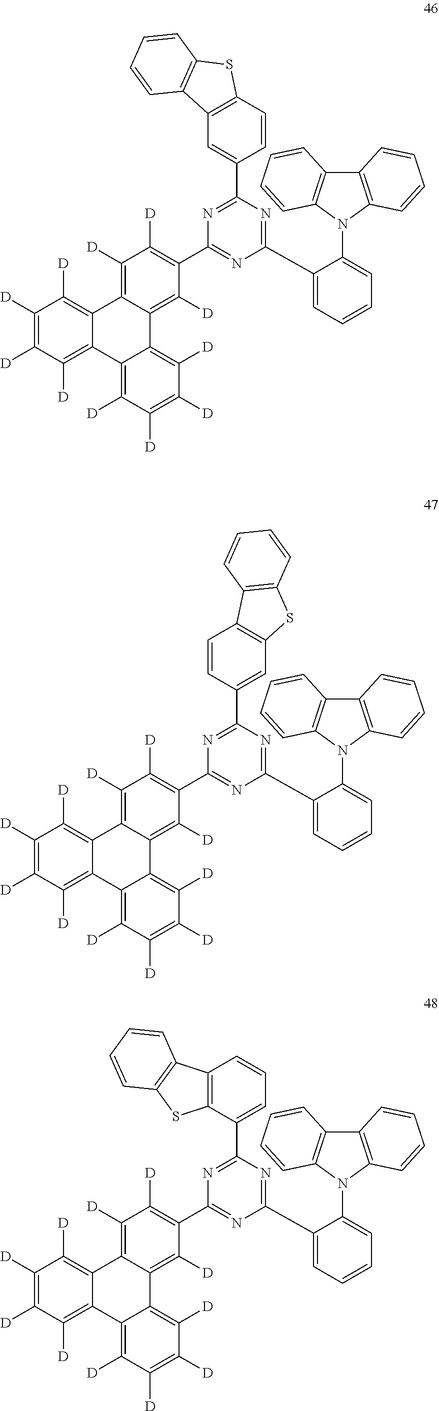 [SMILES: [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5c4sc4ccccc45)n3)c([2H])c([2H])c21]

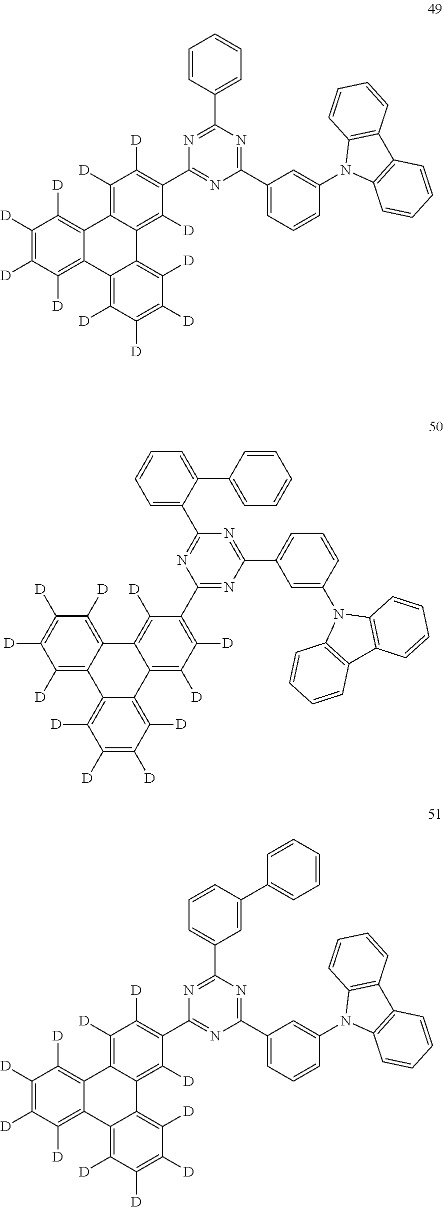 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-n5c6ccccc6c6ccccc65)c4)n3)c([2H])c([2H])c21]

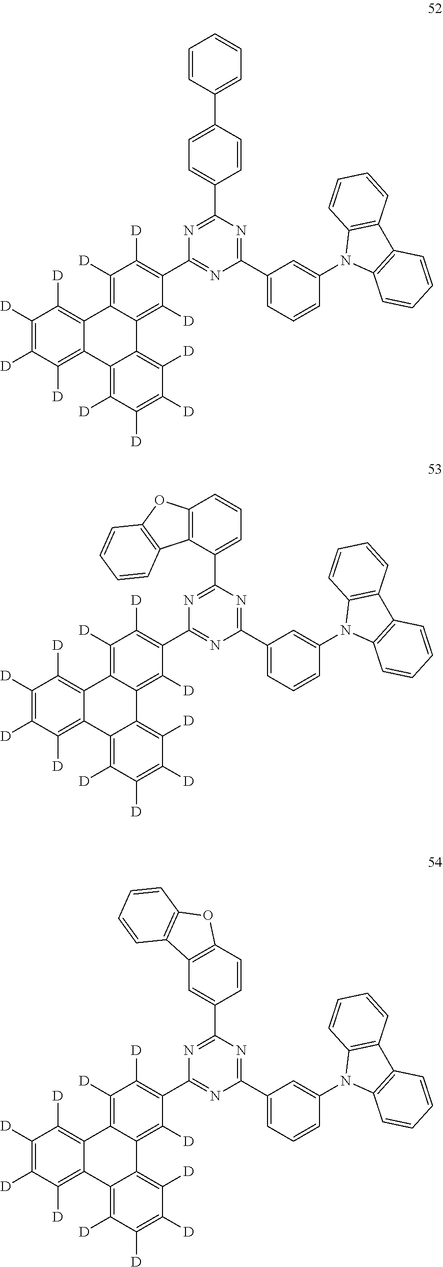 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-n5c6ccccc6c6ccccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5oc6ccccc6c5c4)n3)c([2H])c([2H])c21]

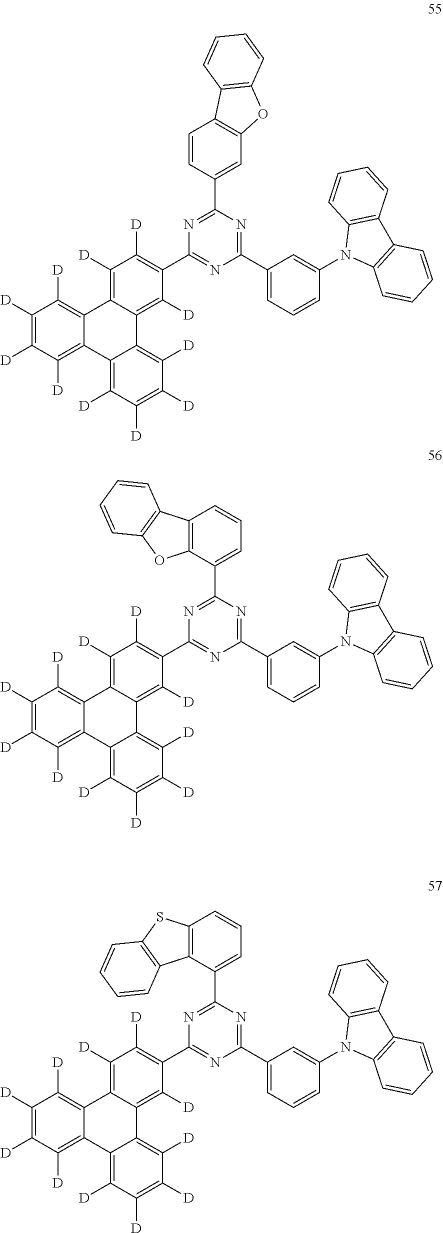 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5sc6ccccc6c45)n3)c([2H])c([2H])c21]

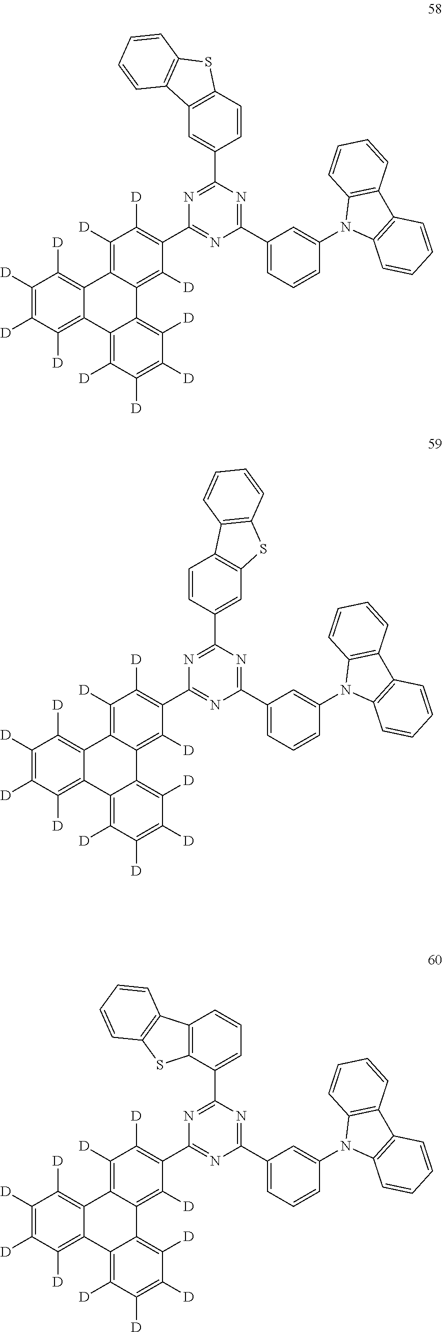 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5c4sc4ccccc45)n3)c([2H])c([2H])c21]

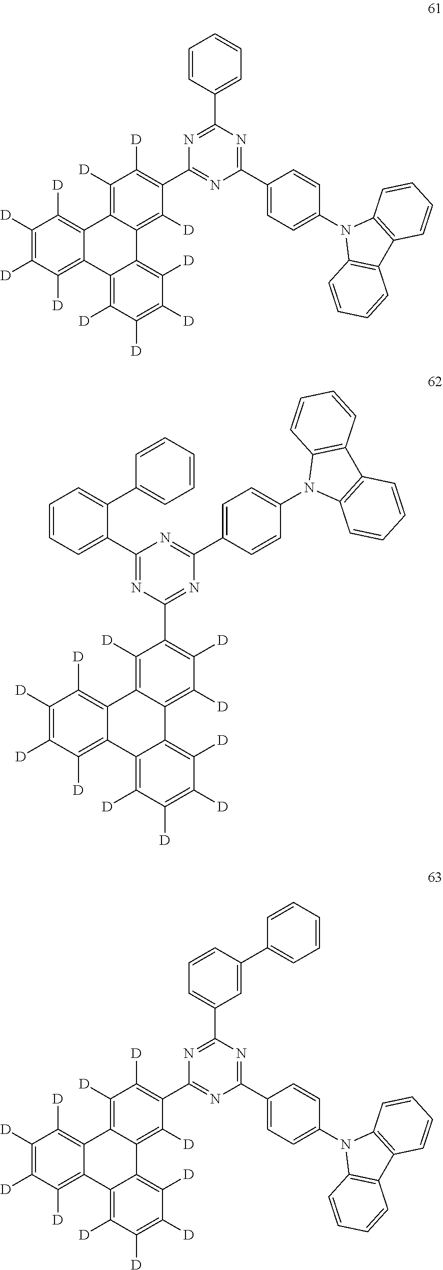 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-n5c6ccccc6c6ccccc65)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc(-c5ccccc5)c4)n3)c([2H])c([2H])c21]

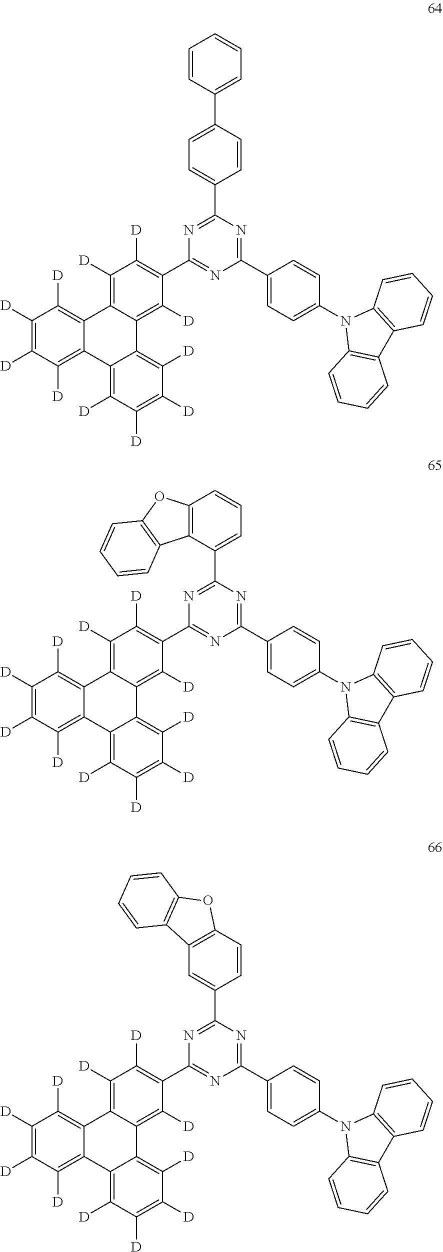 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-n5c6ccccc6c6ccccc65)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5oc6ccccc6c5c4)n3)c([2H])c([2H])c21]

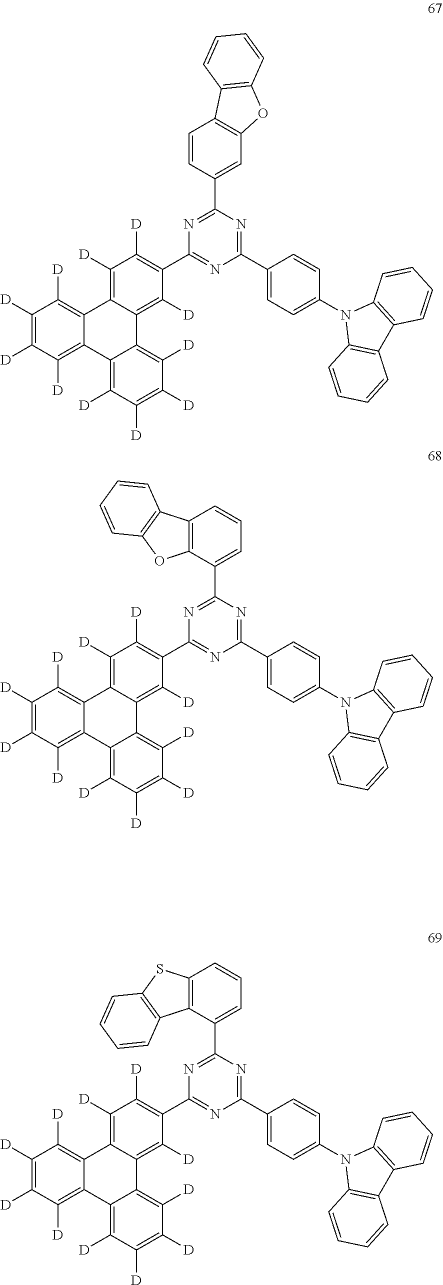 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5sc6ccccc6c45)n3)c([2H])c([2H])c21]

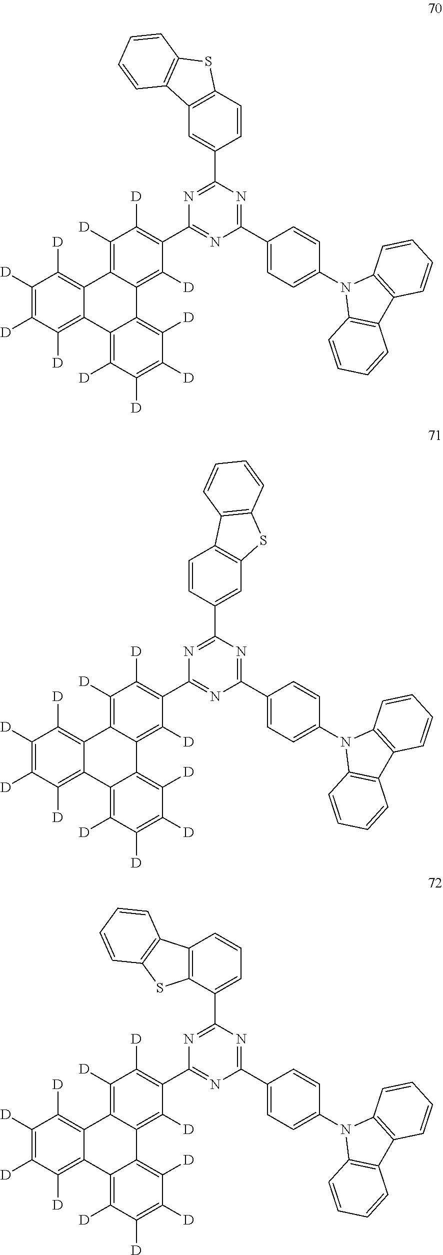 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5c4sc4ccccc45)n3)c([2H])c([2H])c21]

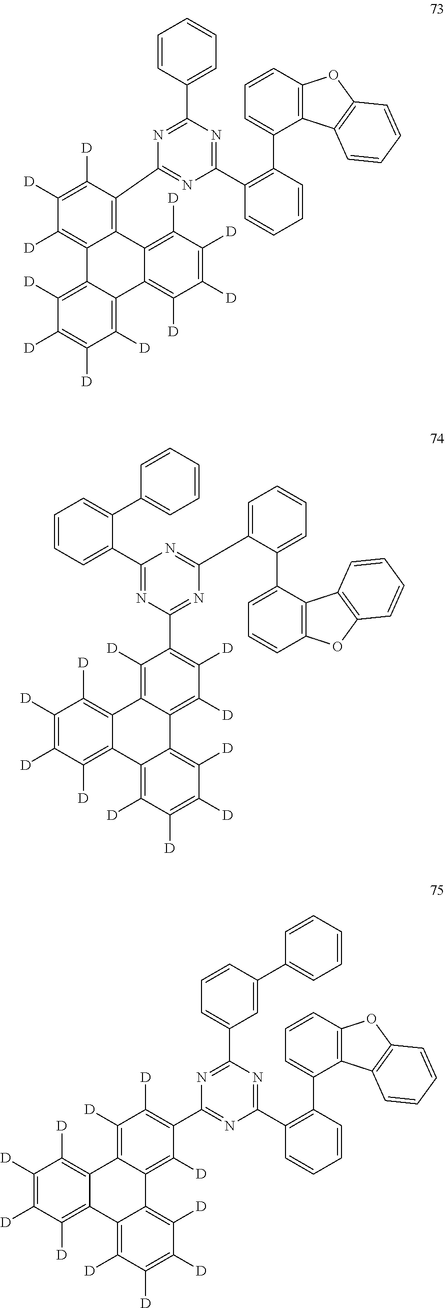 [SMILES: [2H]c1c([2H])c([2H])c2c(c1[2H])c1c([2H])c([2H])c([2H])c([2H])c1c1c(-c3nc(-c4ccccc4)nc(-c4ccccc4-c4cccc5oc6ccccc6c45)n3)c([2H])c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5oc6ccccc6c45)n3)c([2H])c([2H])c21]

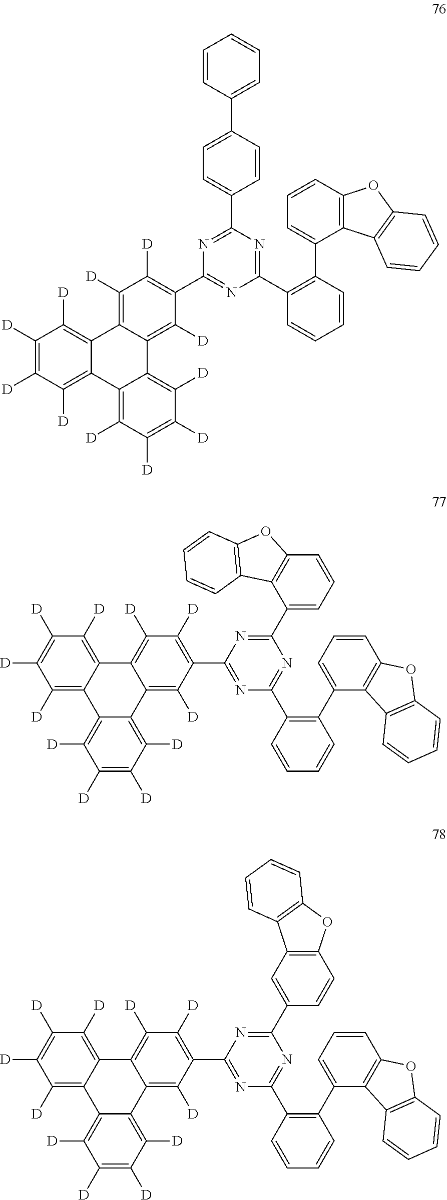 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5oc6ccccc6c45)n3)c([2H])c([2H])c21]

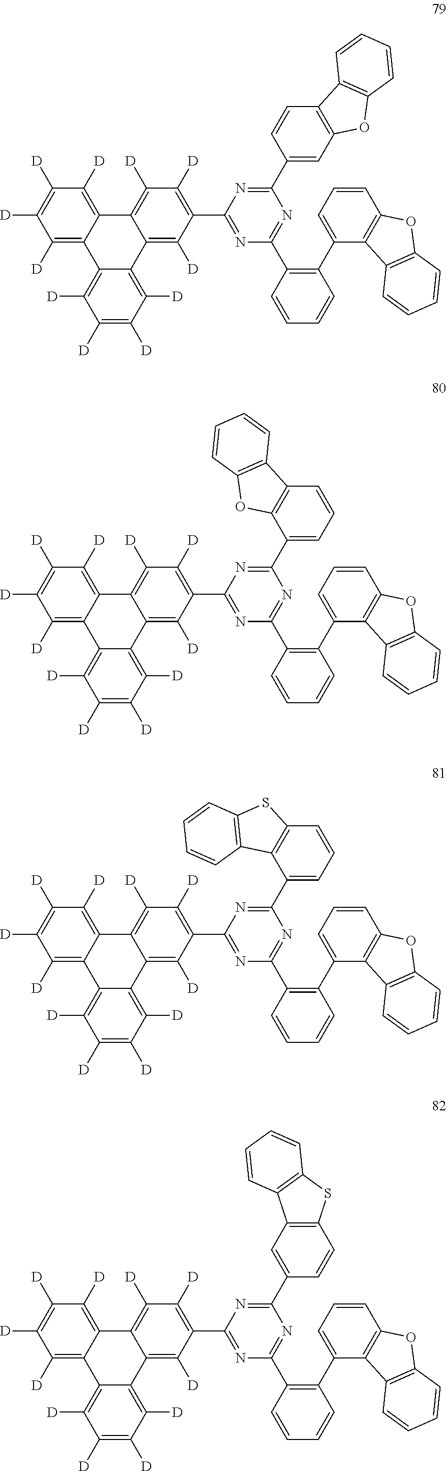

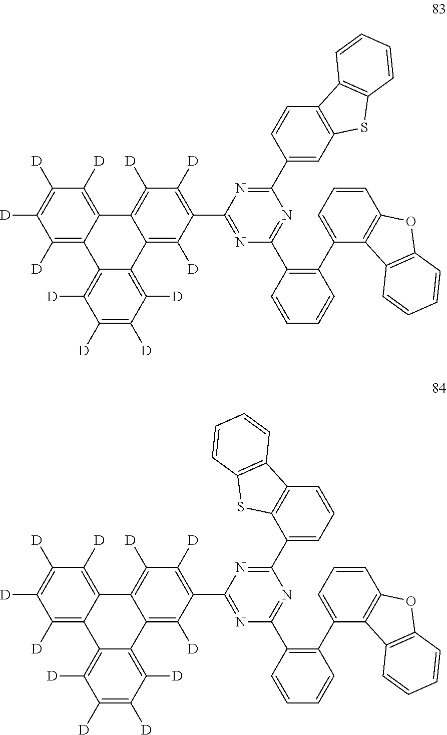 [SMILES: [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5c4sc4ccccc45)n3)c([2H])c([2H])c21]

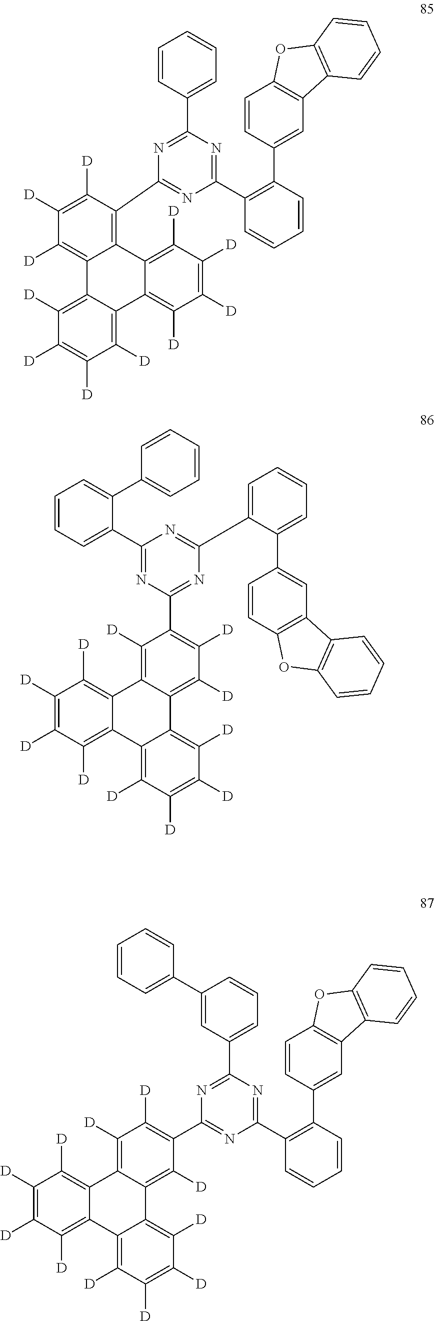 [SMILES: [2H]c1c([2H])c([2H])c2c(c1[2H])c1c([2H])c([2H])c([2H])c([2H])c1c1c(-c3nc(-c4ccccc4)nc(-c4ccccc4-c4ccc5oc6ccccc6c5c4)n3)c([2H])c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5oc6ccccc6c5c4)n3)c([2H])c([2H])c21]

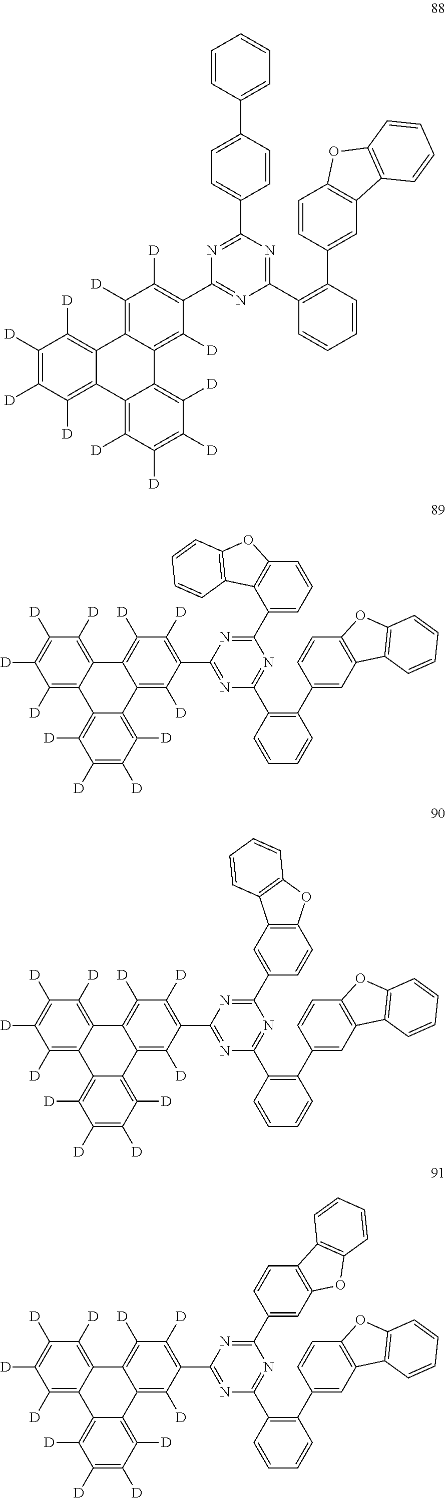

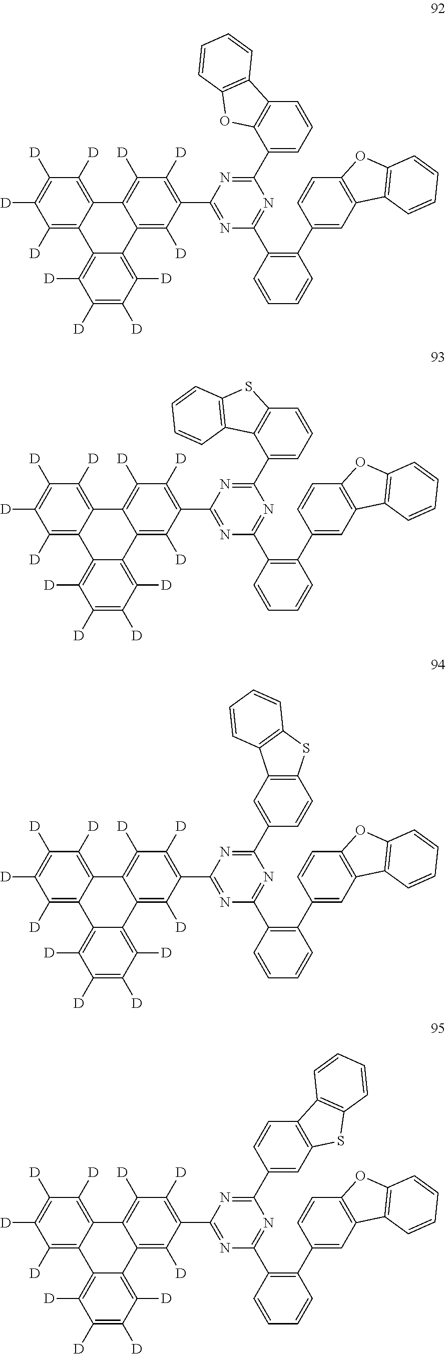

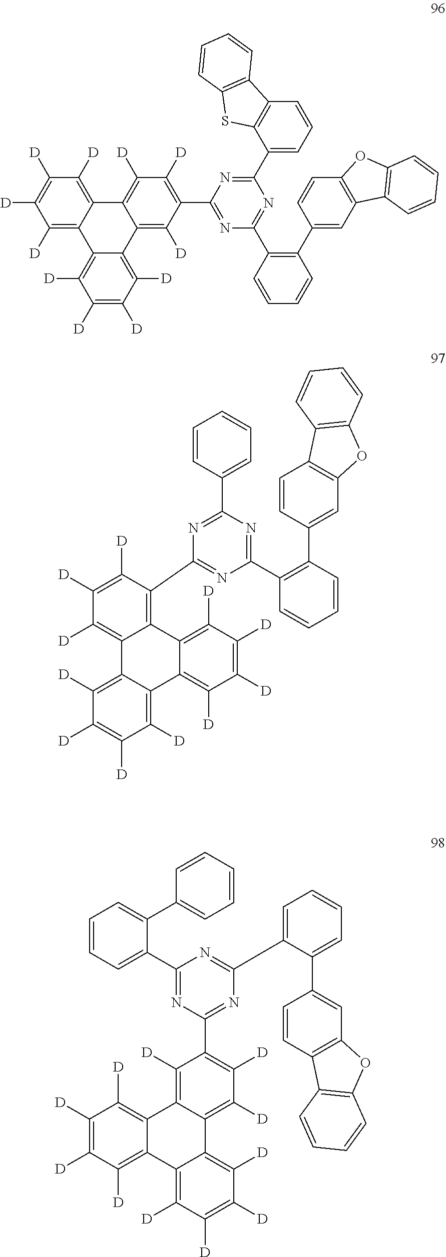 [SMILES: [2H]c1c([2H])c([2H])c2c(c1[2H])c1c([2H])c([2H])c([2H])c([2H])c1c1c([2H])c(-c3nc(-c4ccccc4-c4ccc5oc6ccccc6c5c4)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c(-c3nc(-c4ccccc4)nc(-c4ccccc4-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c(c4)oc4ccccc45)n3)c([2H])c([2H])c21]

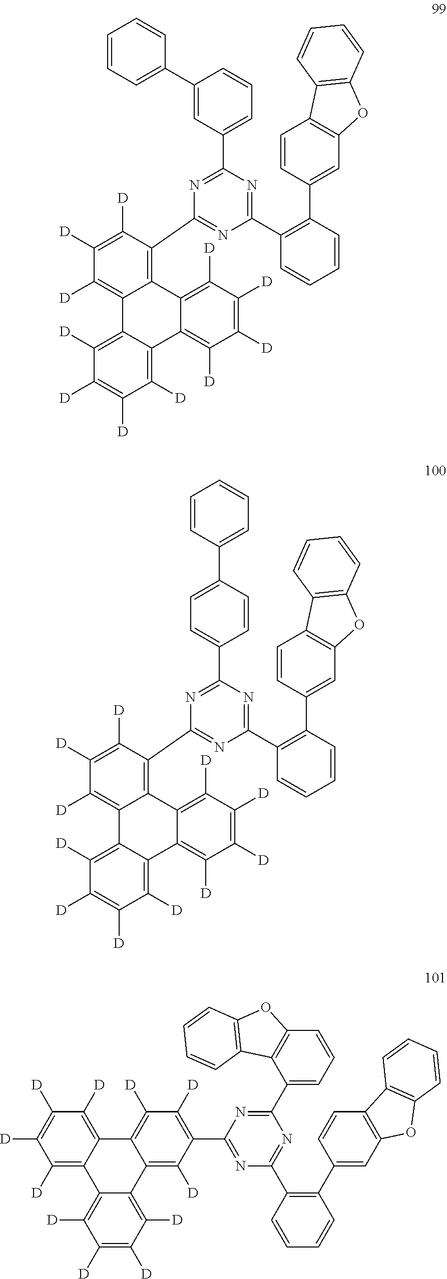 [SMILES: [2H]c1c([2H])c([2H])c2c(c1[2H])c1c([2H])c([2H])c([2H])c([2H])c1c1c(-c3nc(-c4cccc(-c5ccccc5)c4)nc(-c4ccccc4-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-c5ccccc5)cc4)nc(-c4ccccc4-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5oc6ccccc6c45)n3)c([2H])c([2H])c21]

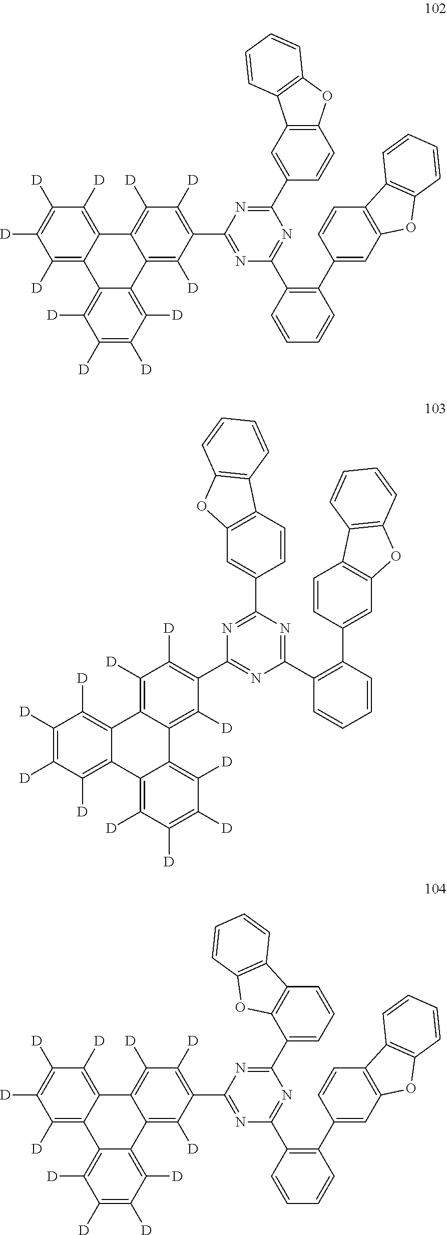 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5c4oc4ccccc45)n3)c([2H])c([2H])c21]

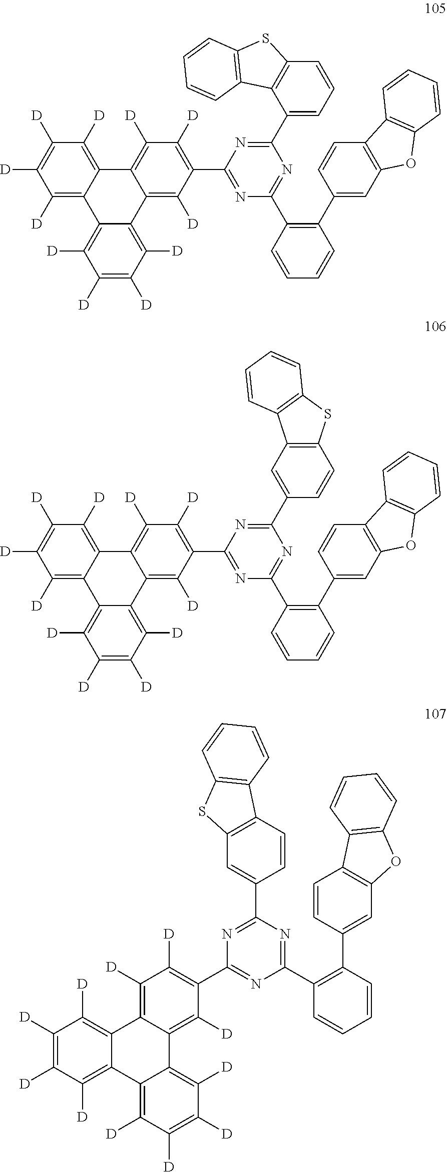 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4ccc5c(c4)oc4ccccc45)n3)c([2H])c([2H])c21]

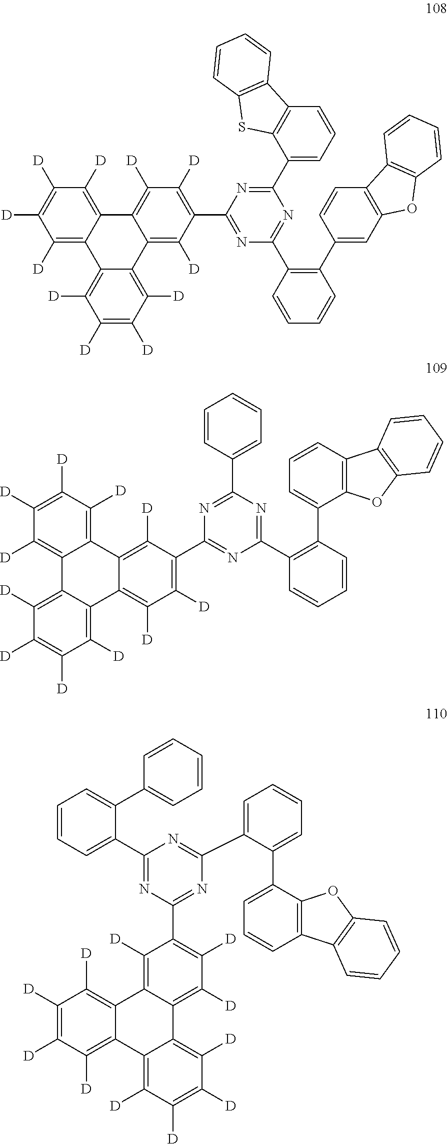 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c4oc4ccccc45)n3)c([2H])c([2H])c21]

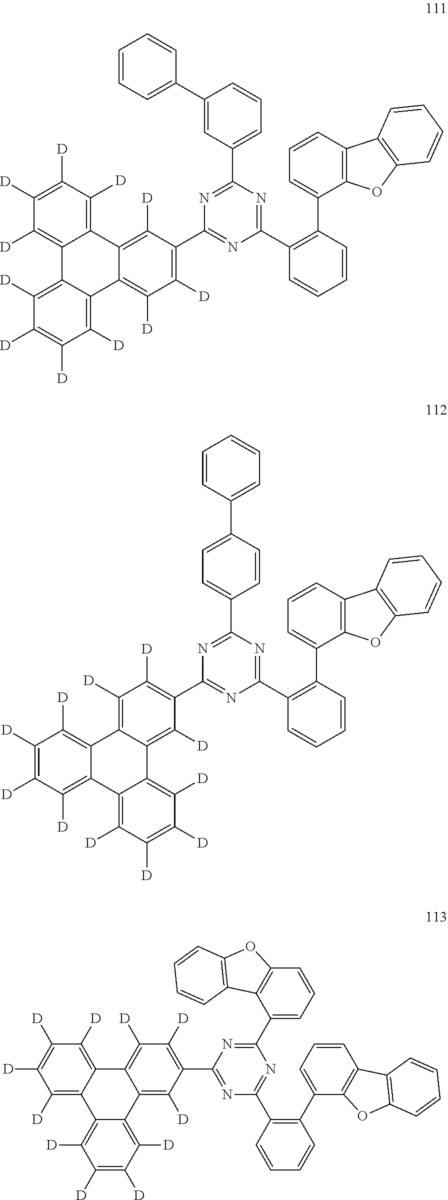 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5oc6ccccc6c45)n3)c([2H])c([2H])c21]

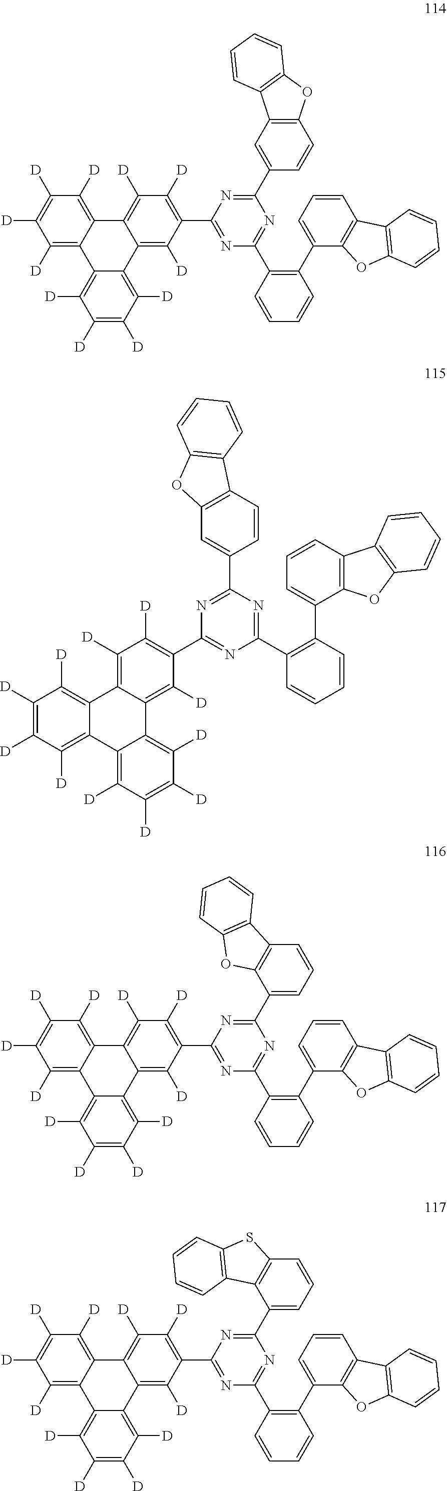

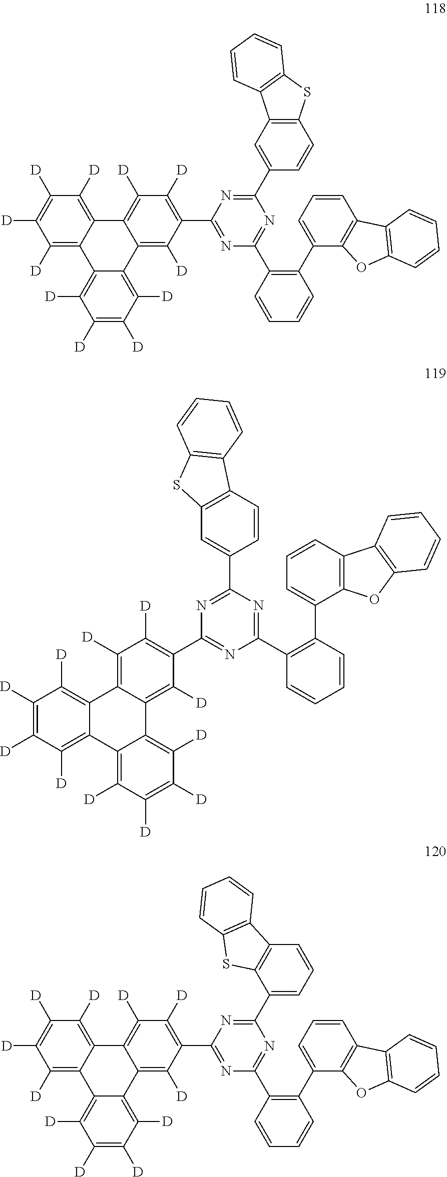 [SMILES: [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5c4sc4ccccc45)n3)c([2H])c([2H])c21]

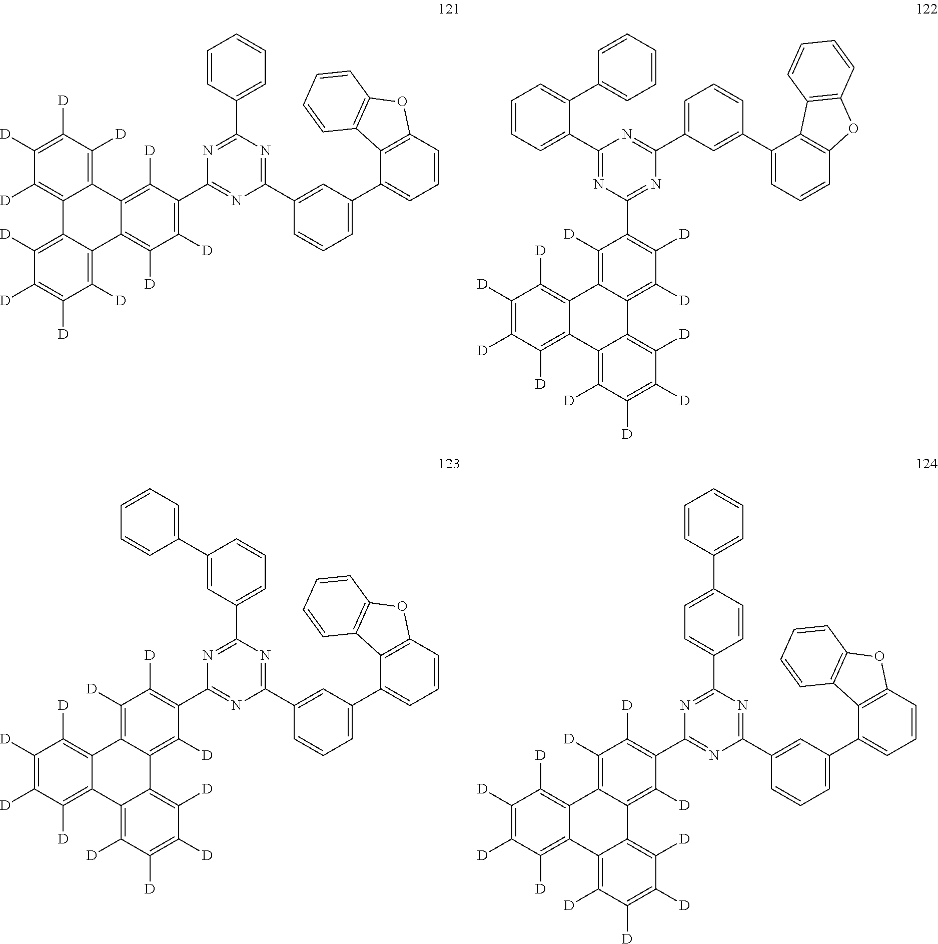

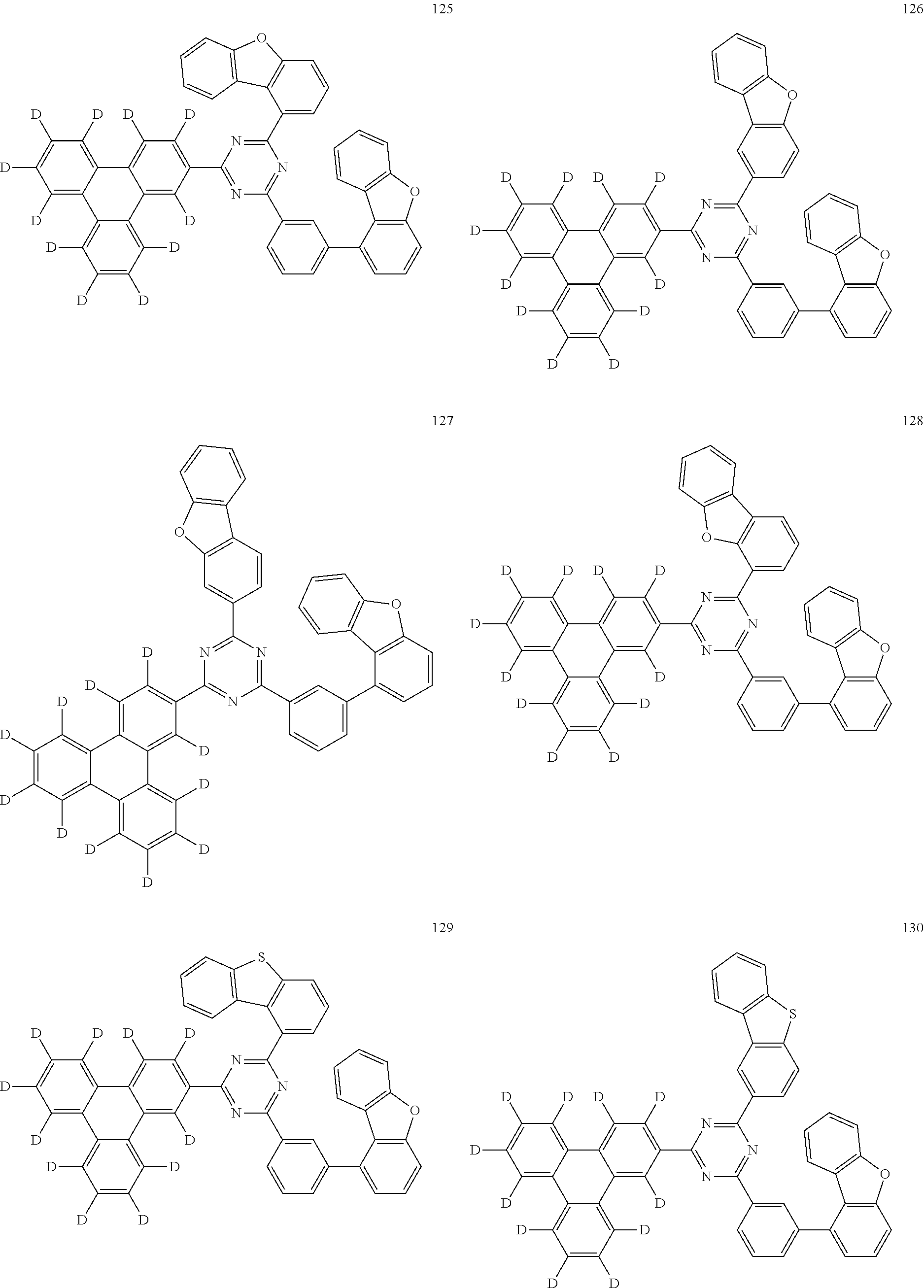

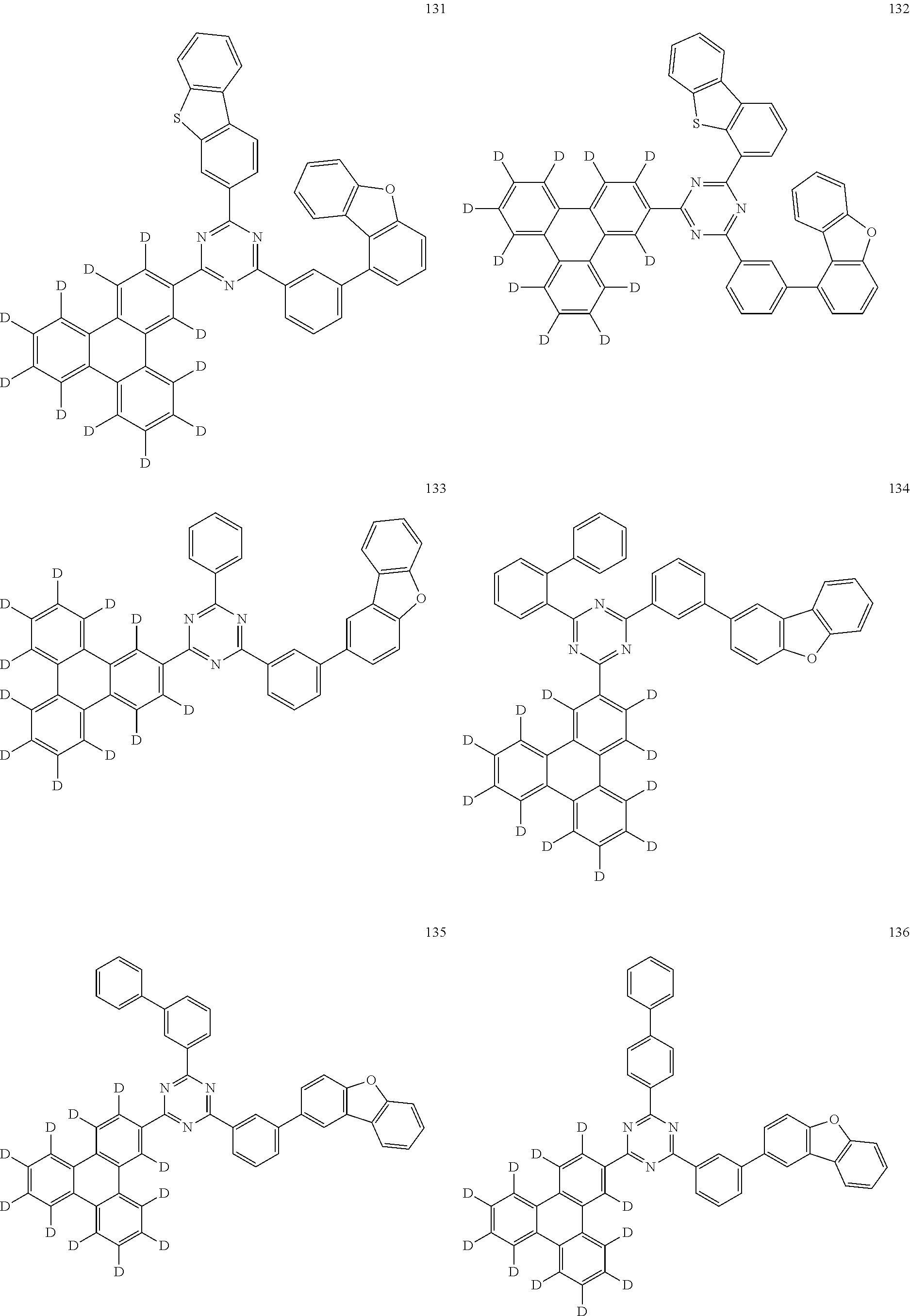

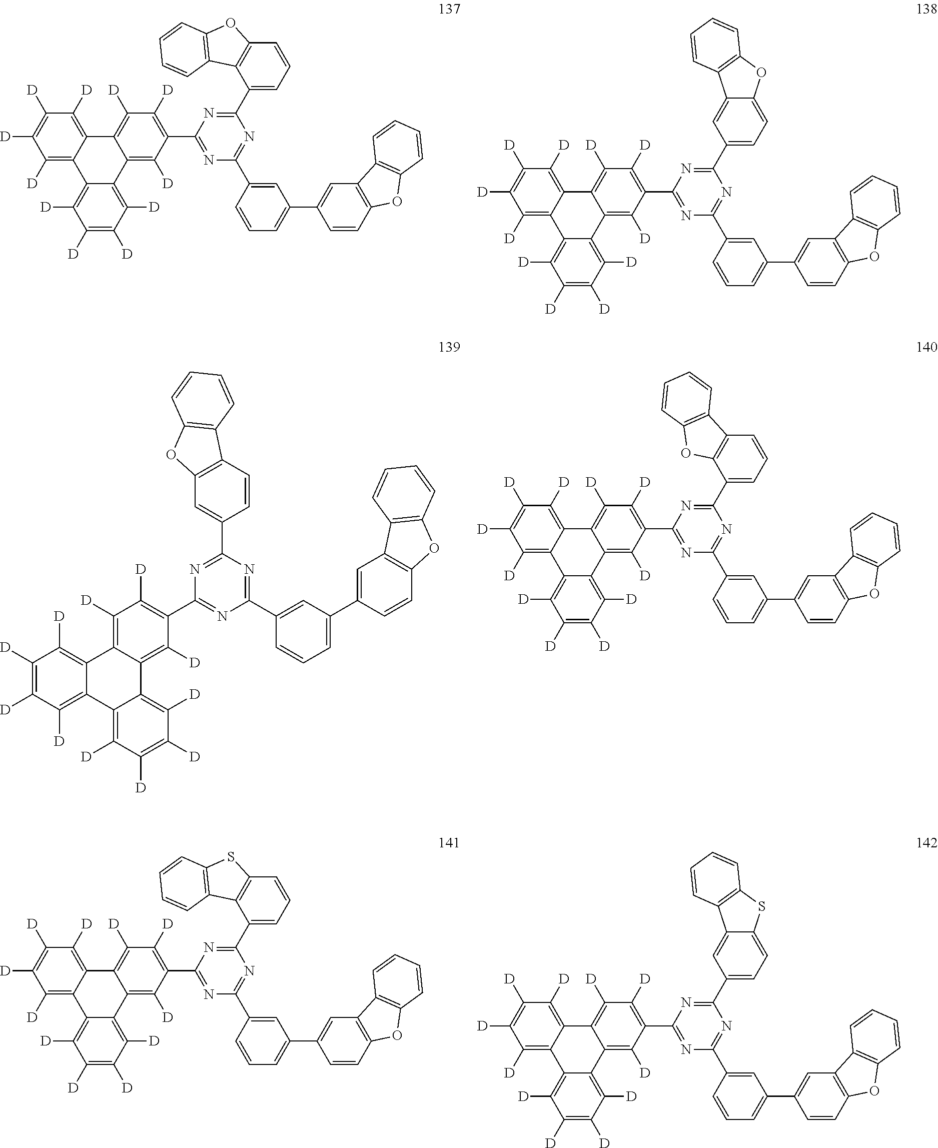

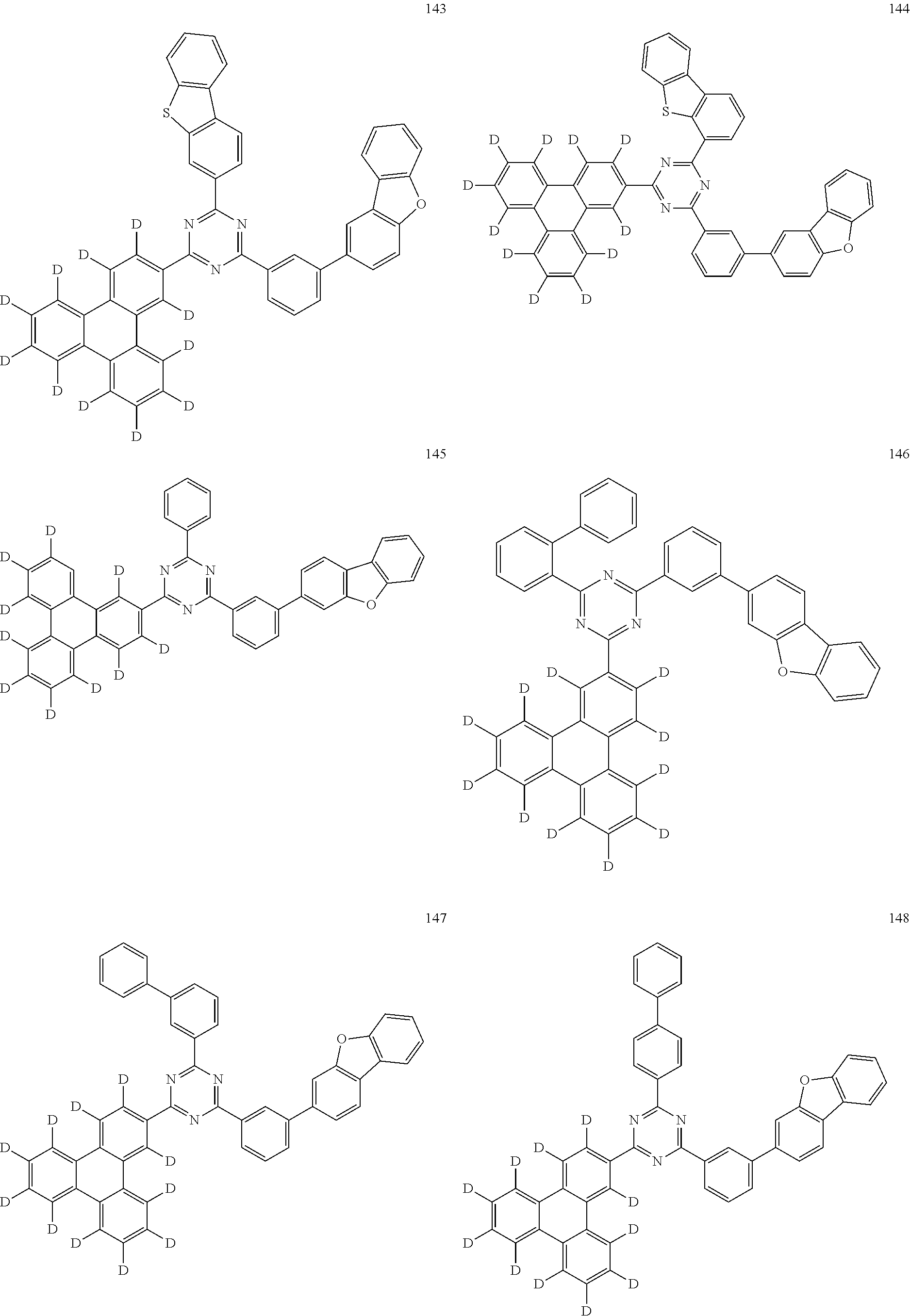

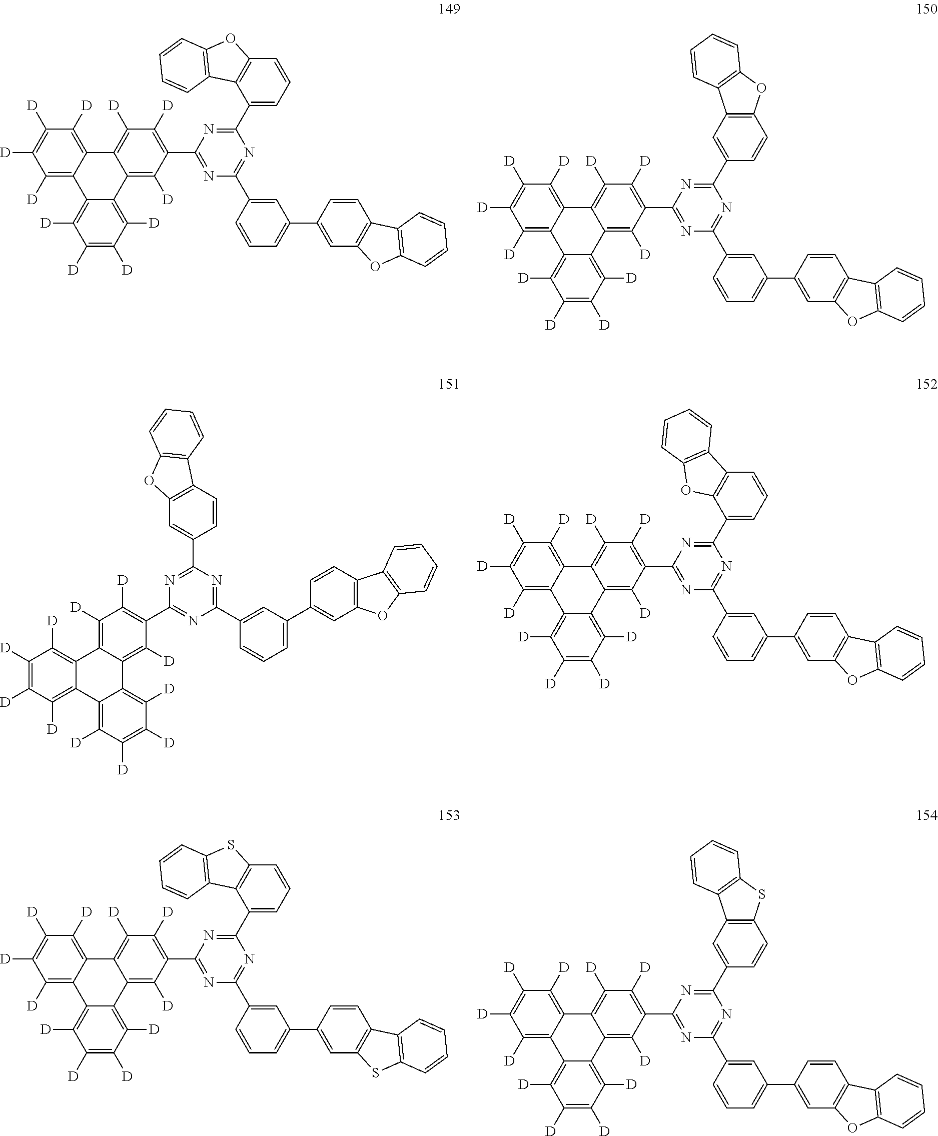

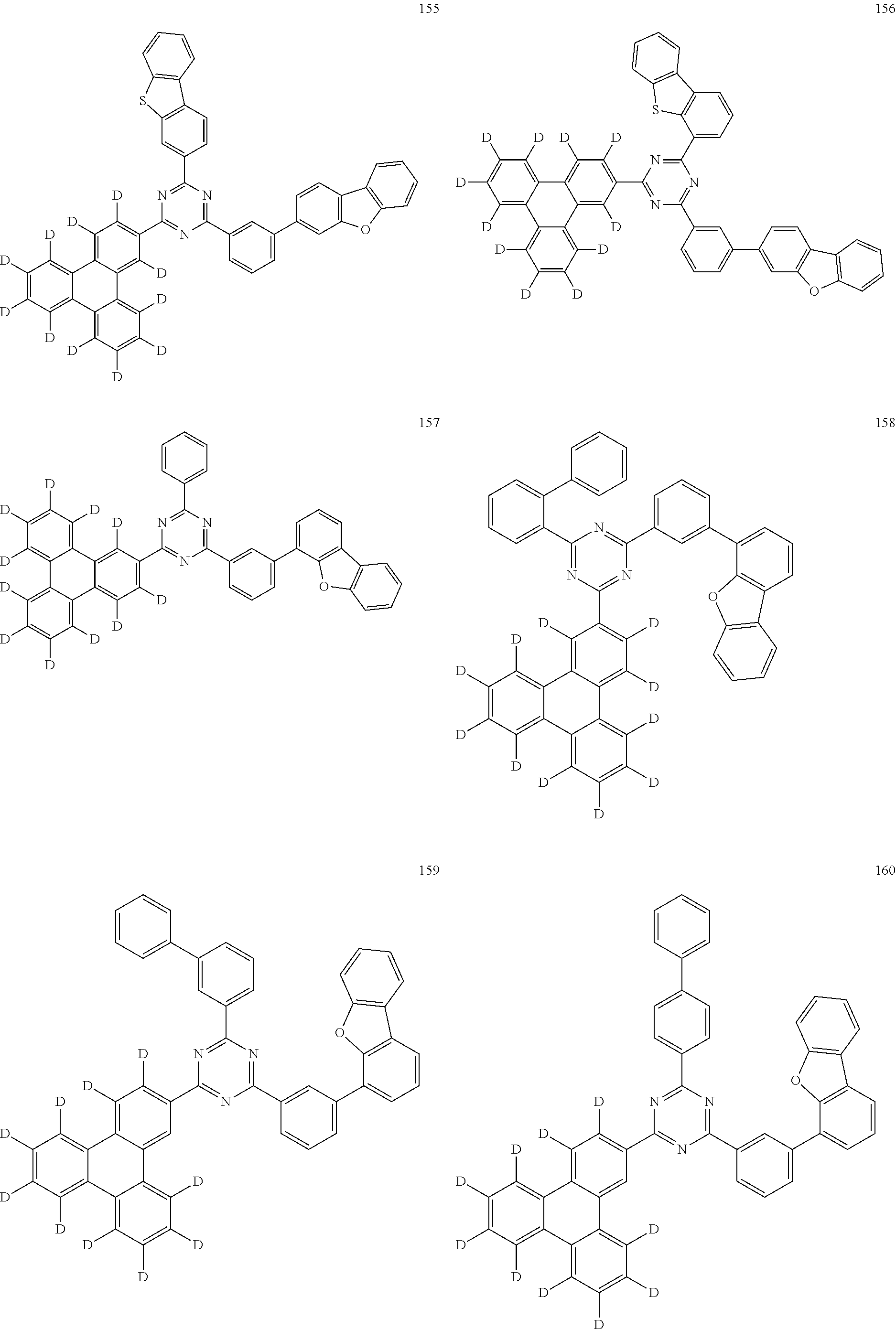

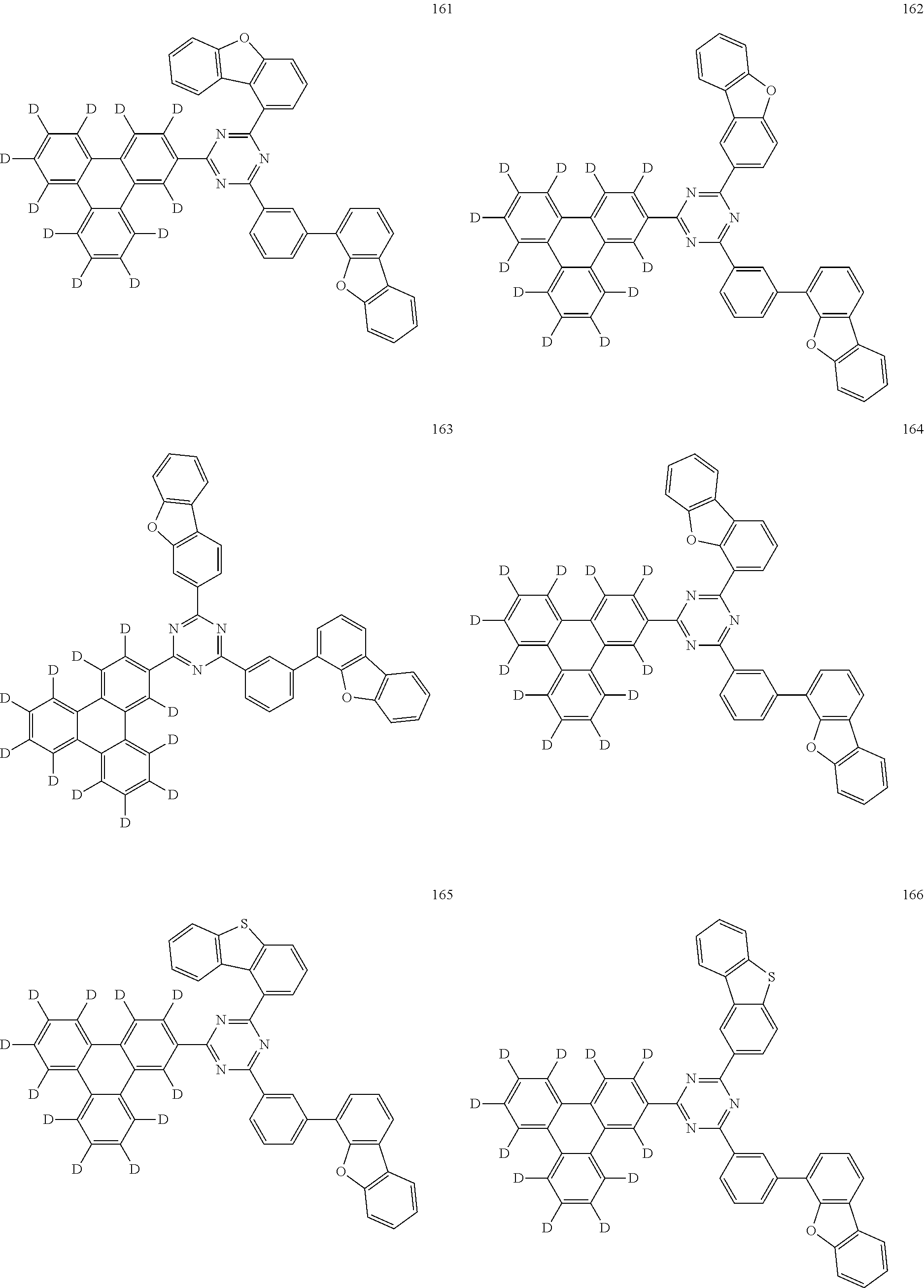

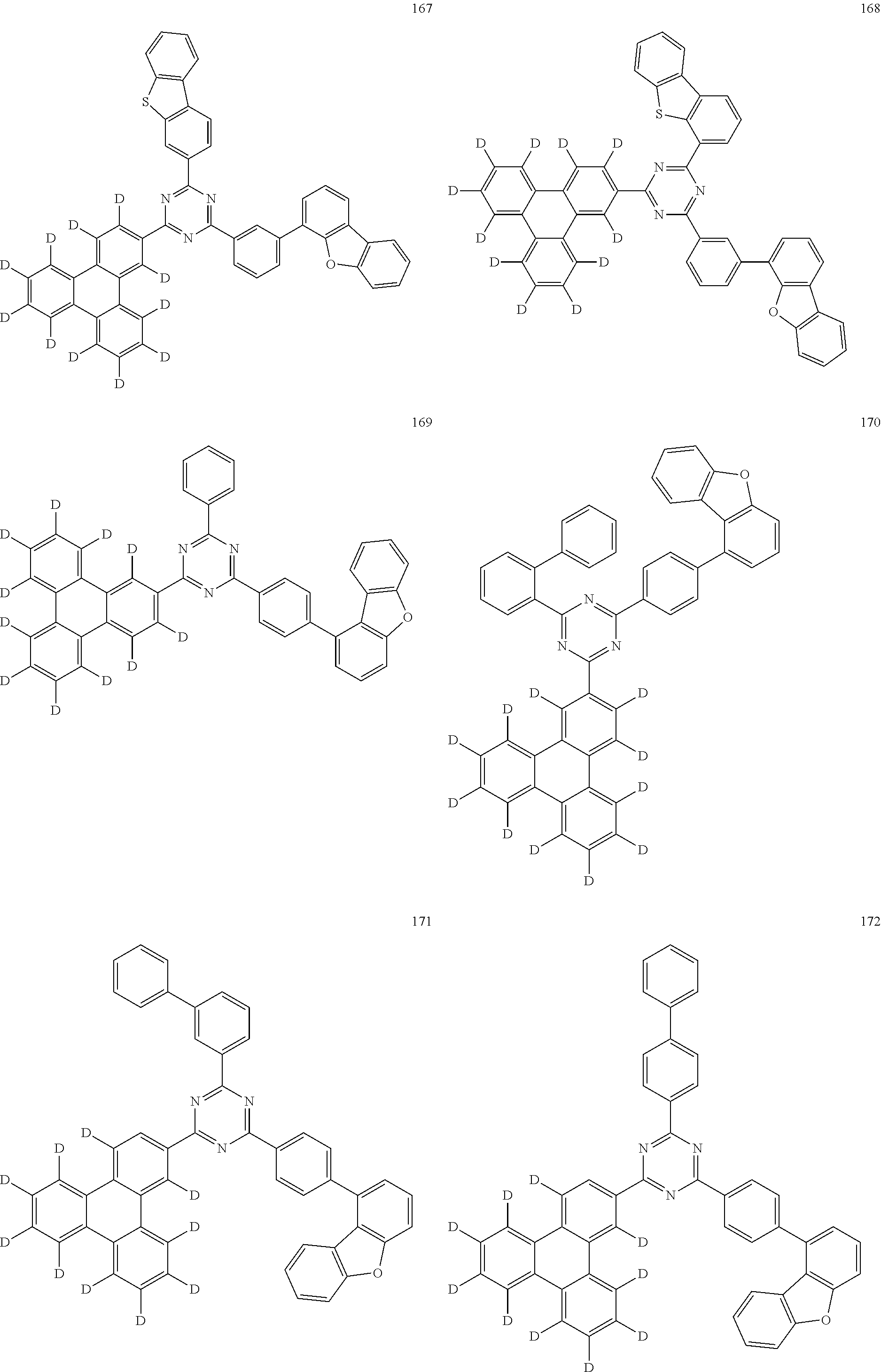

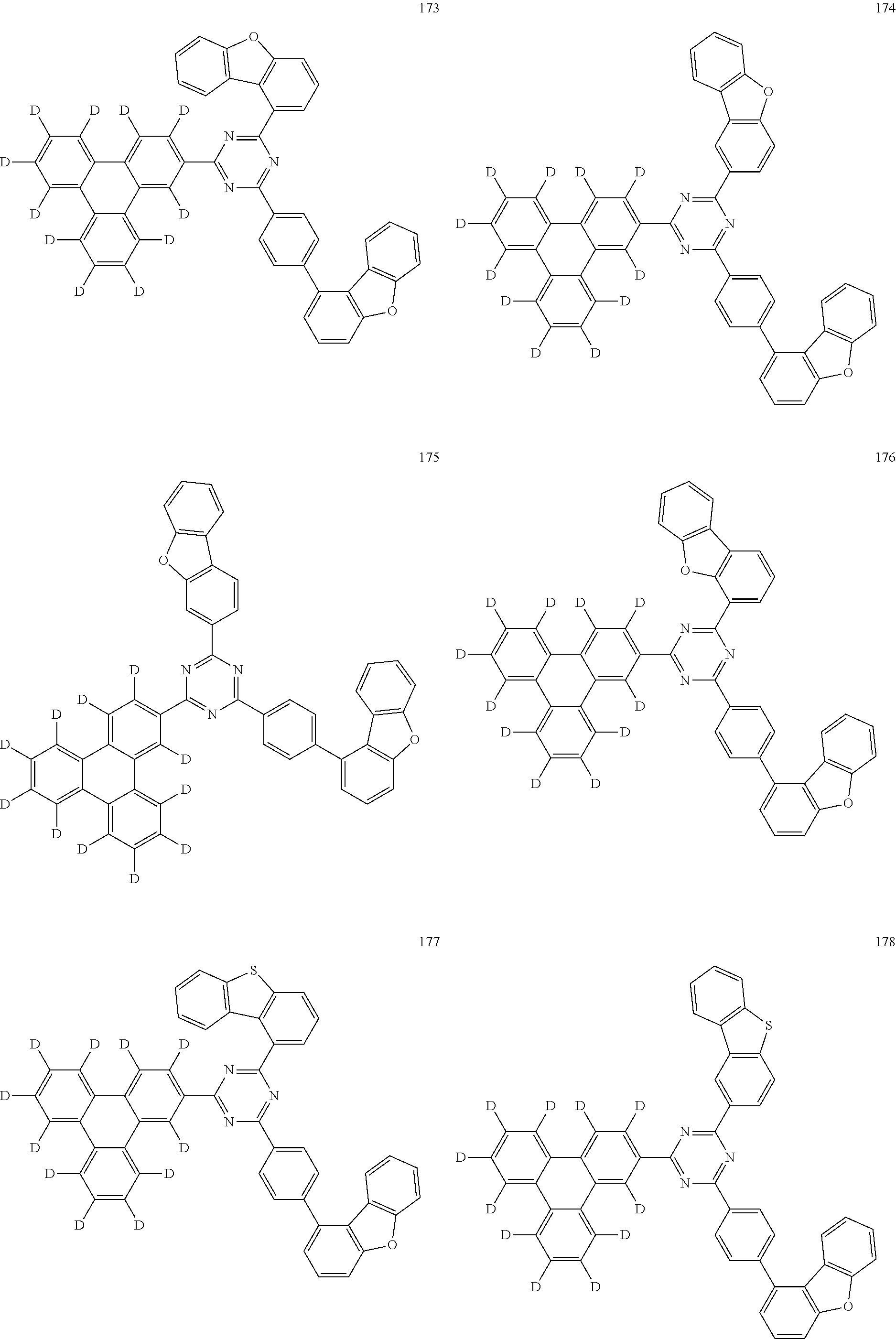

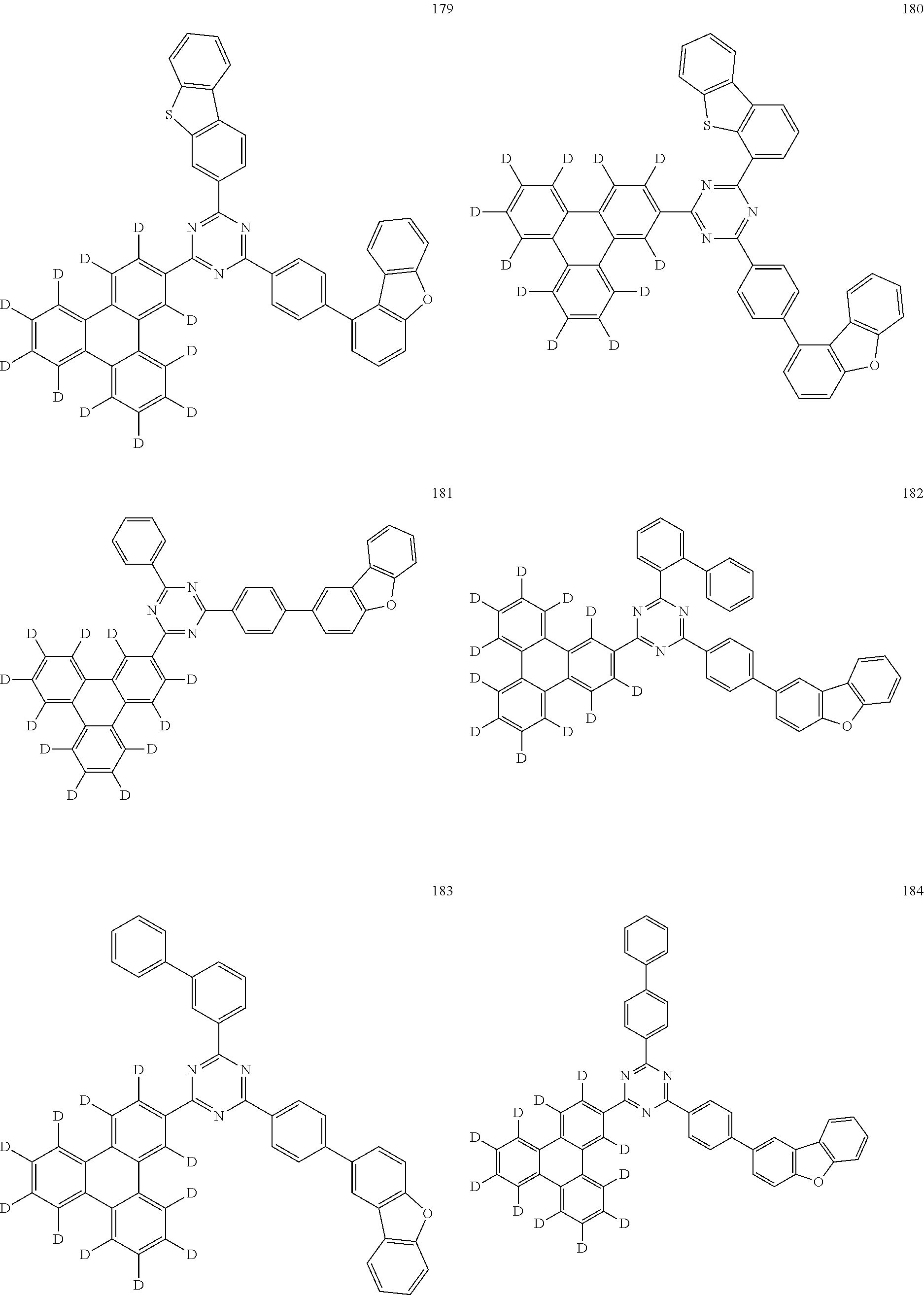

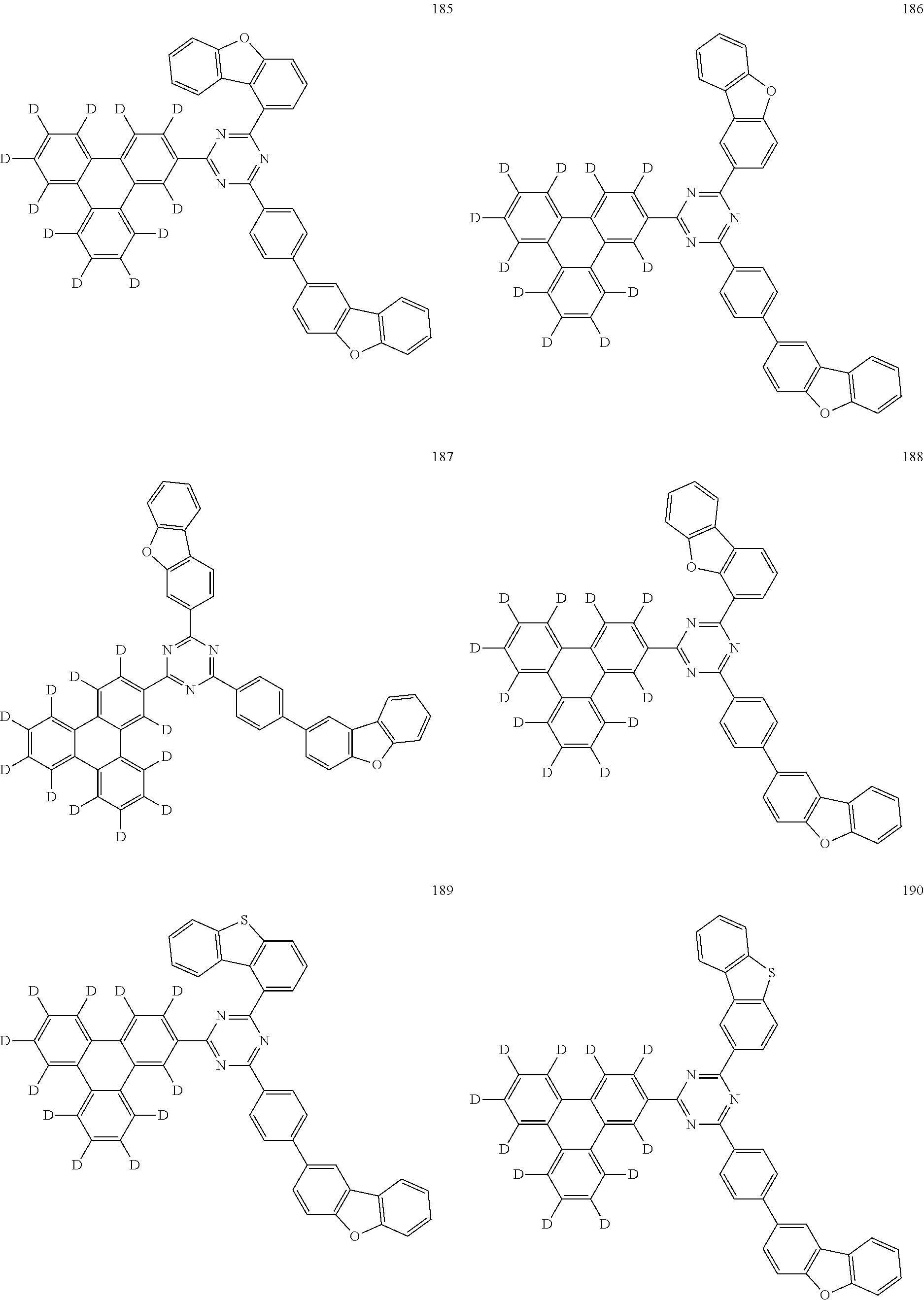

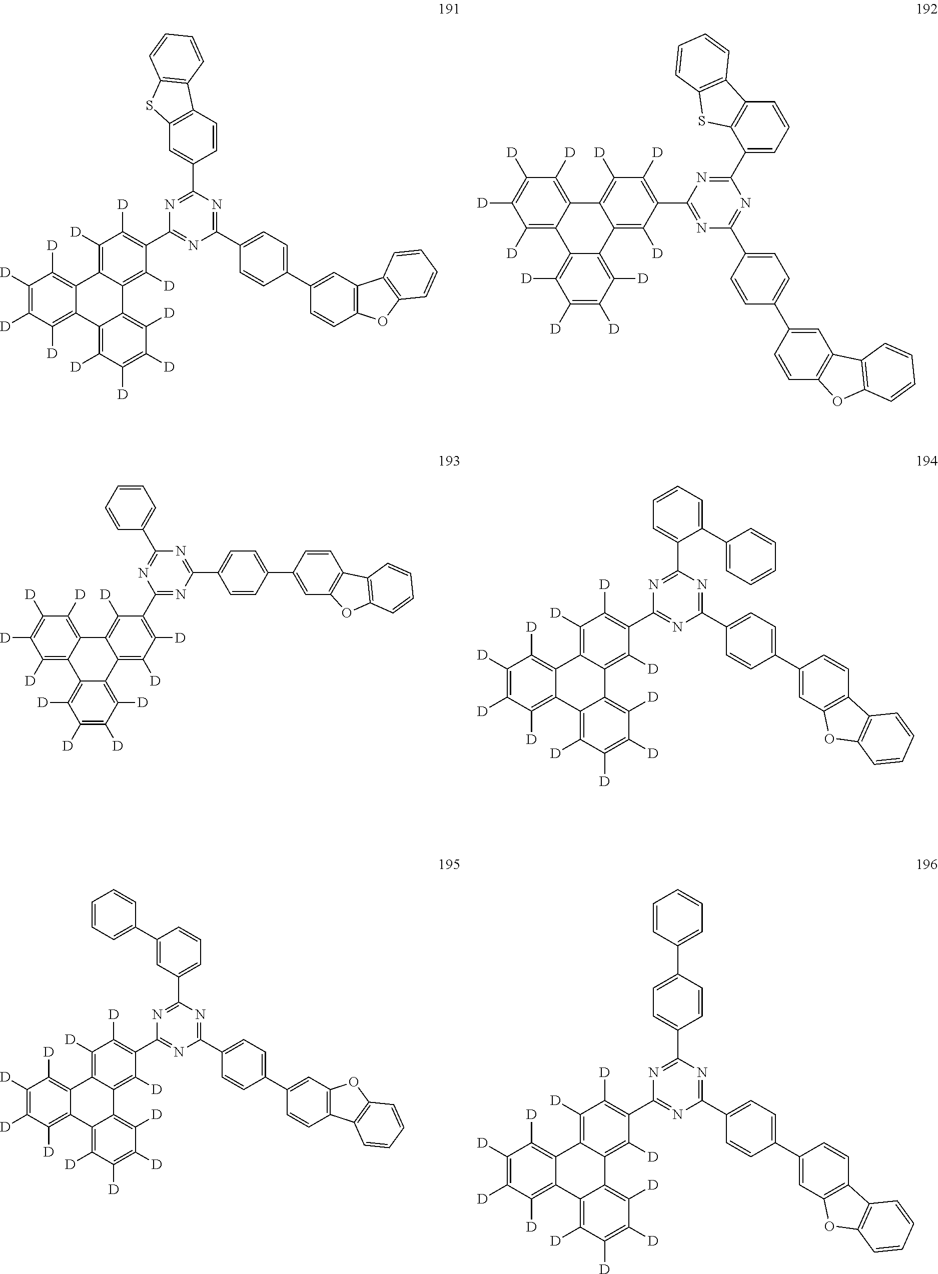

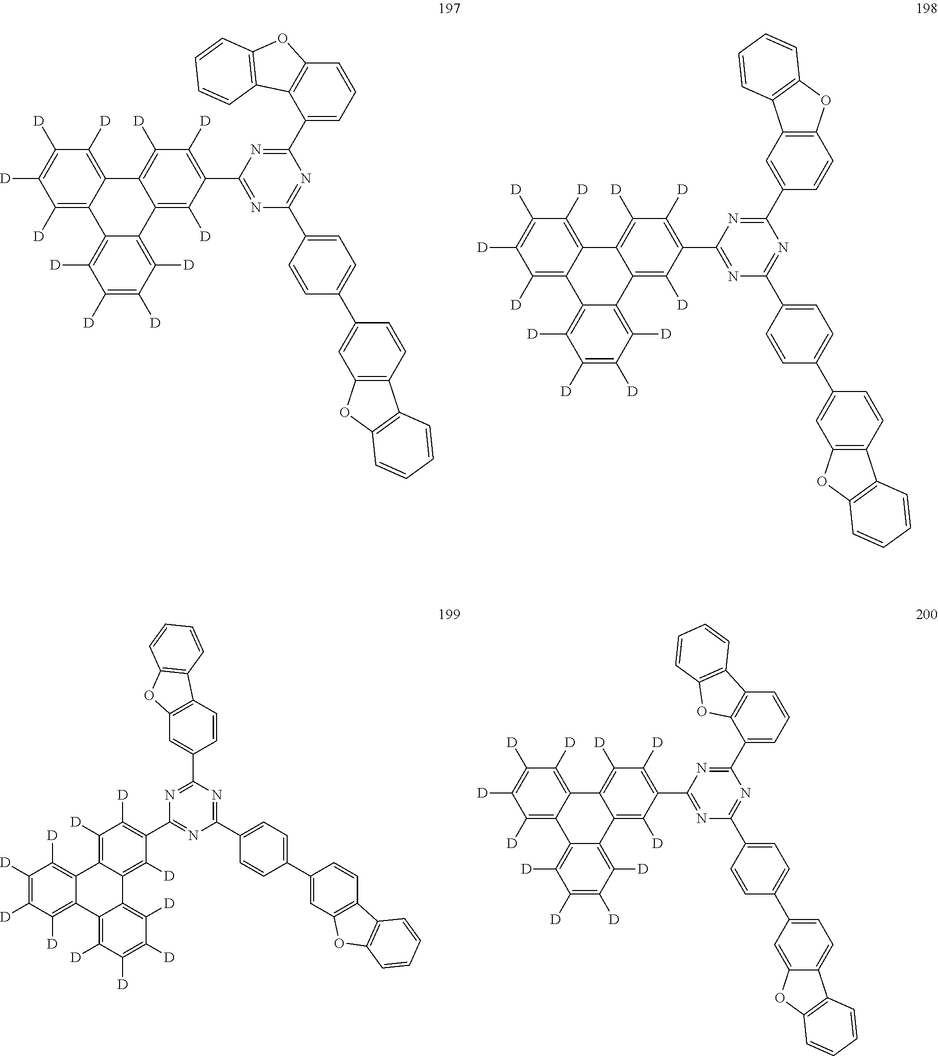

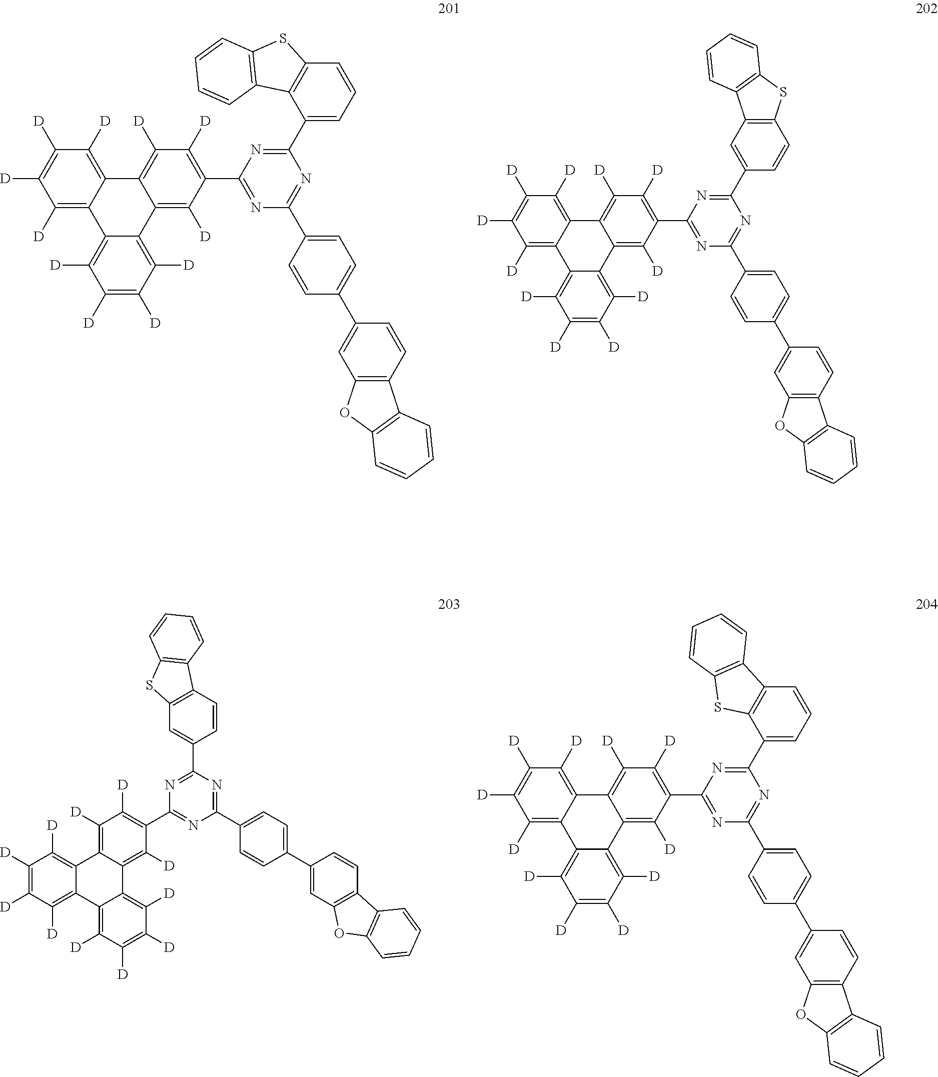

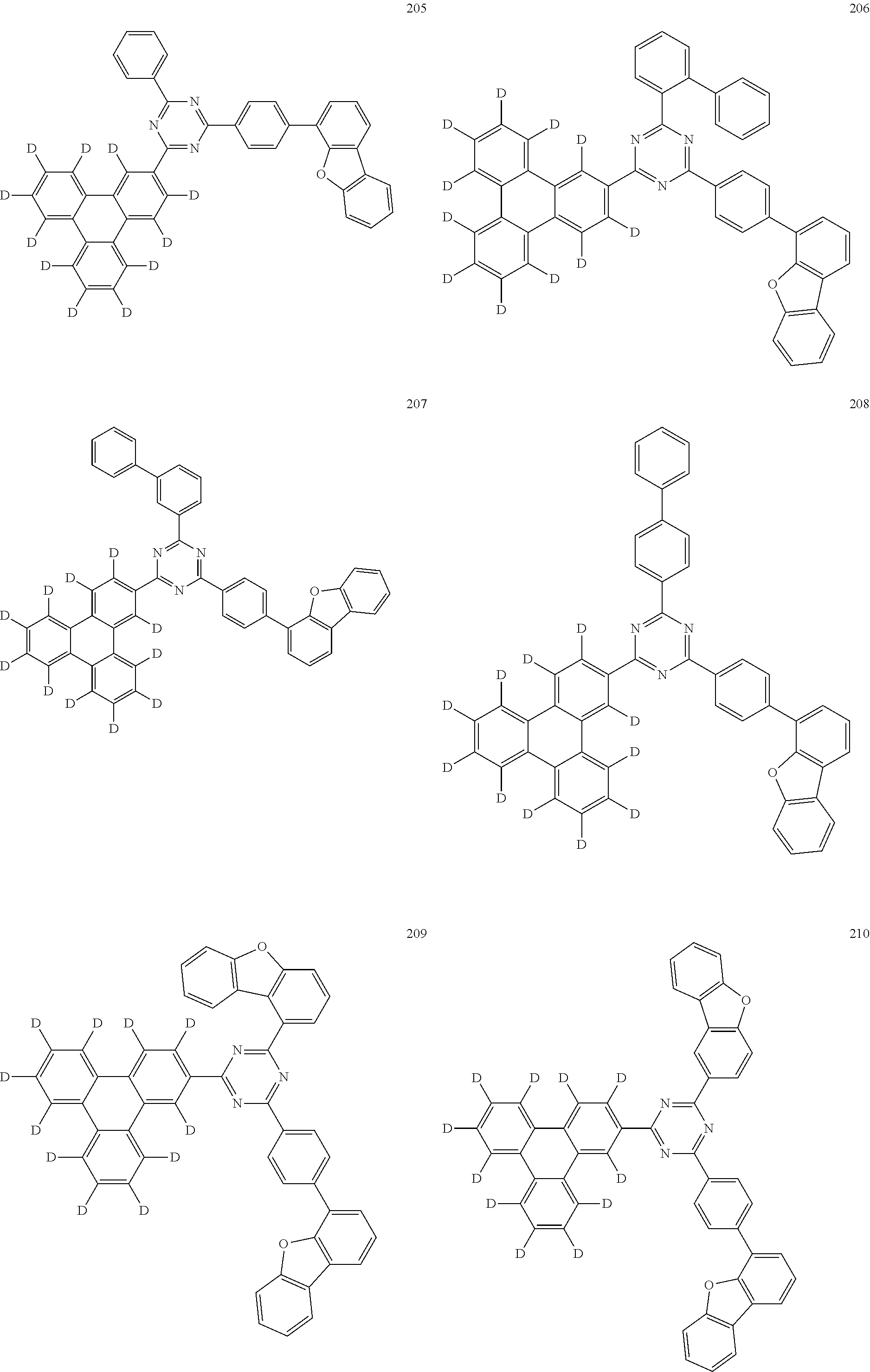

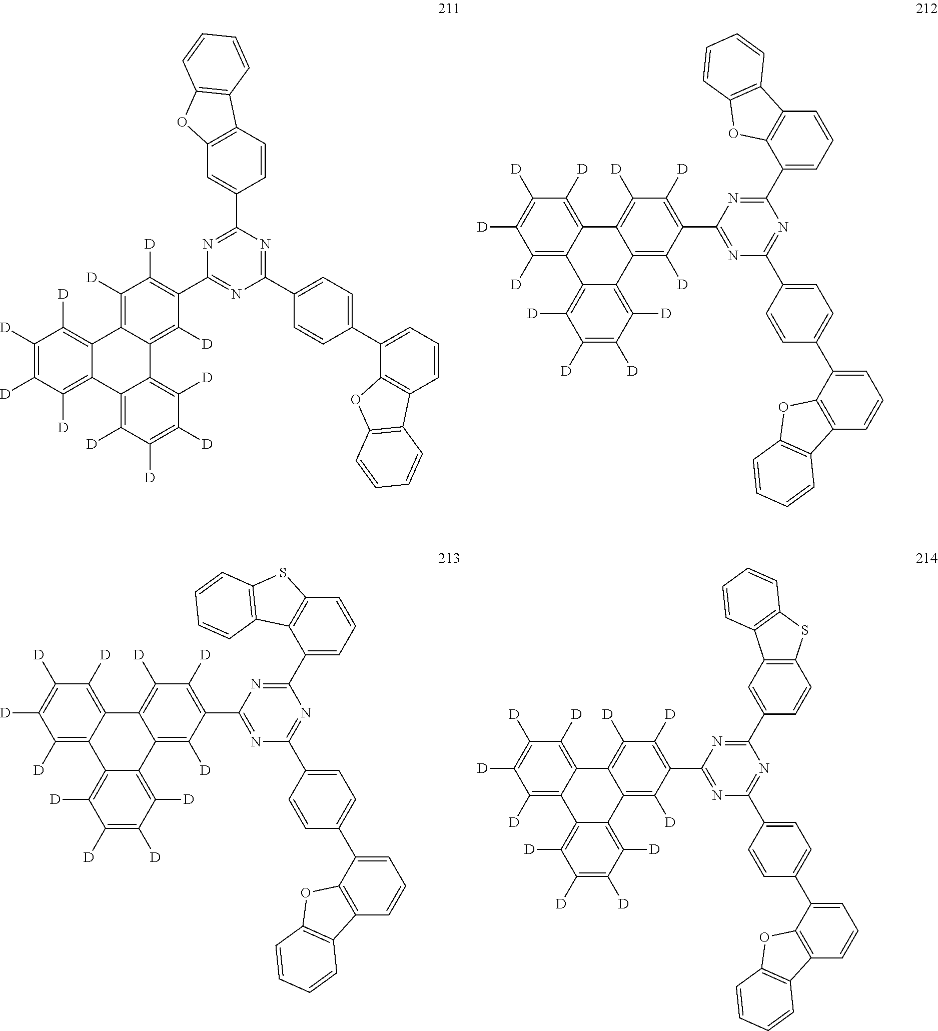

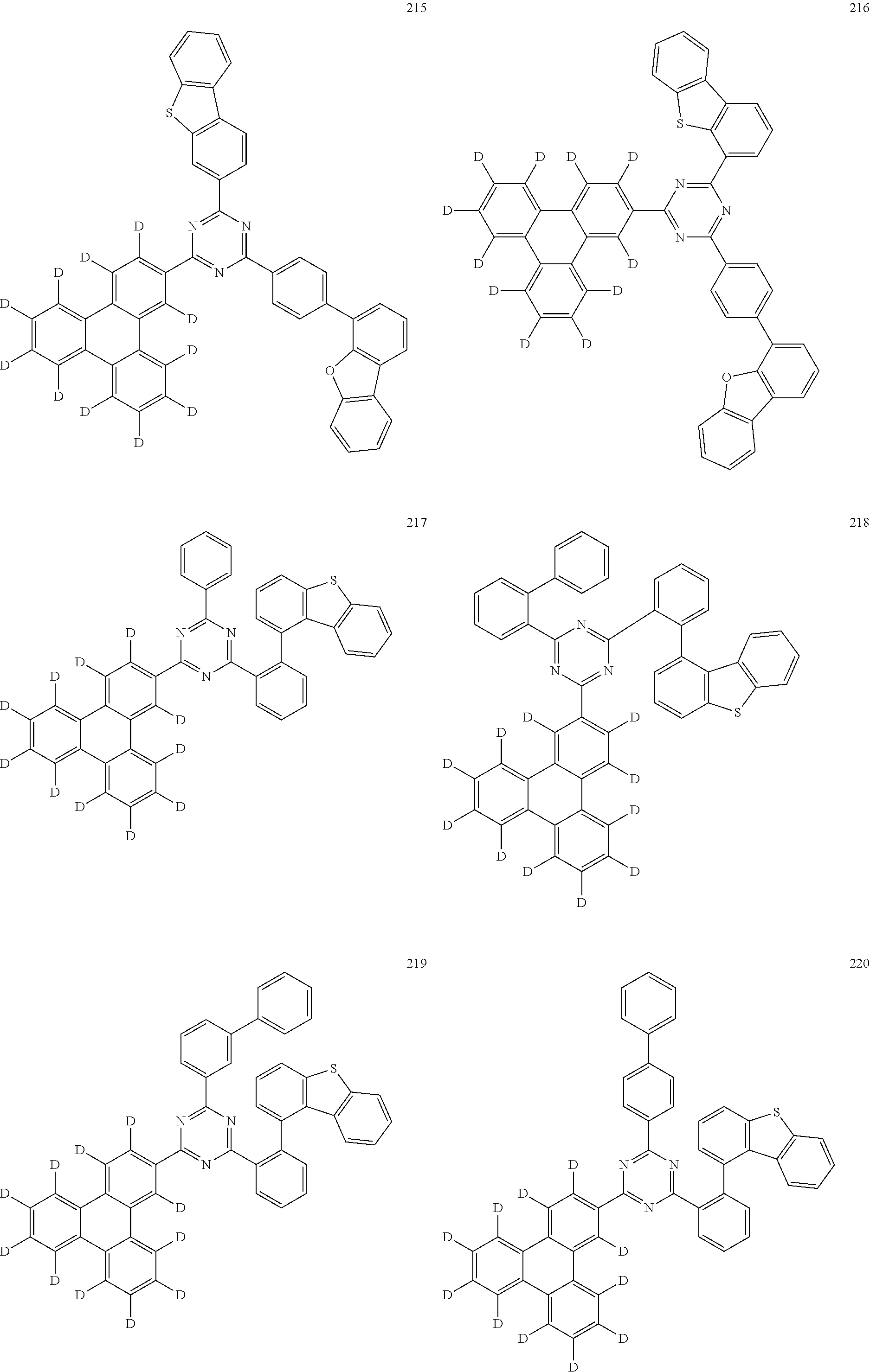

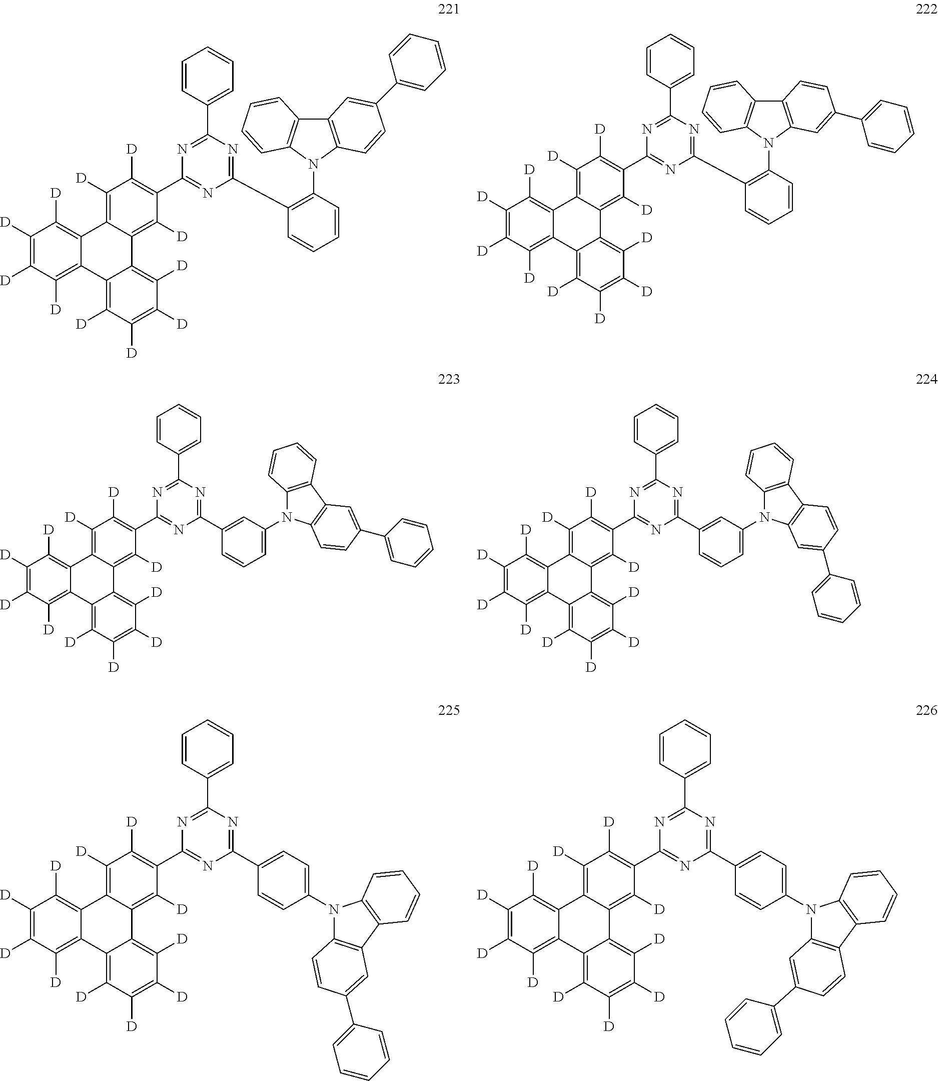

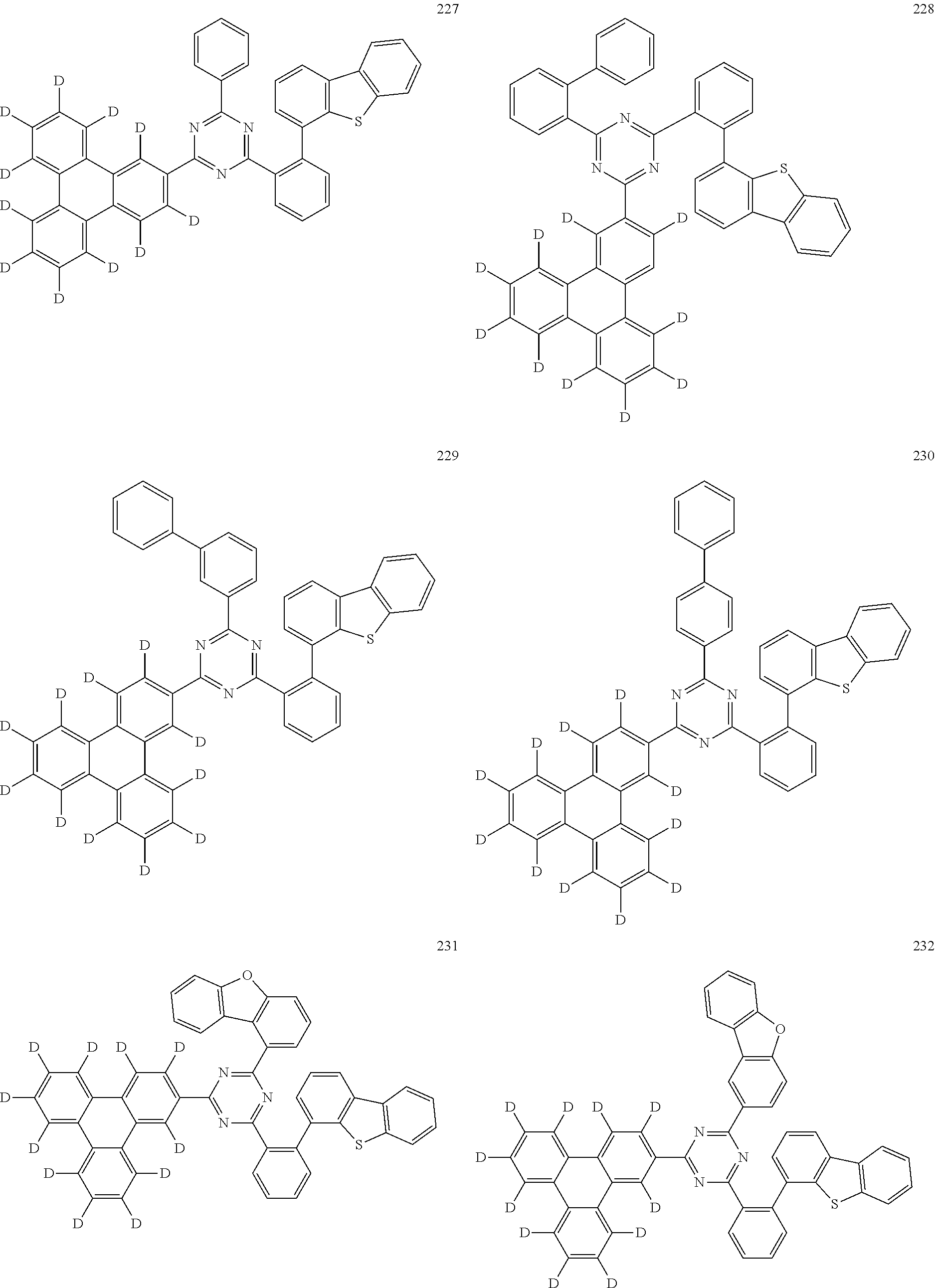

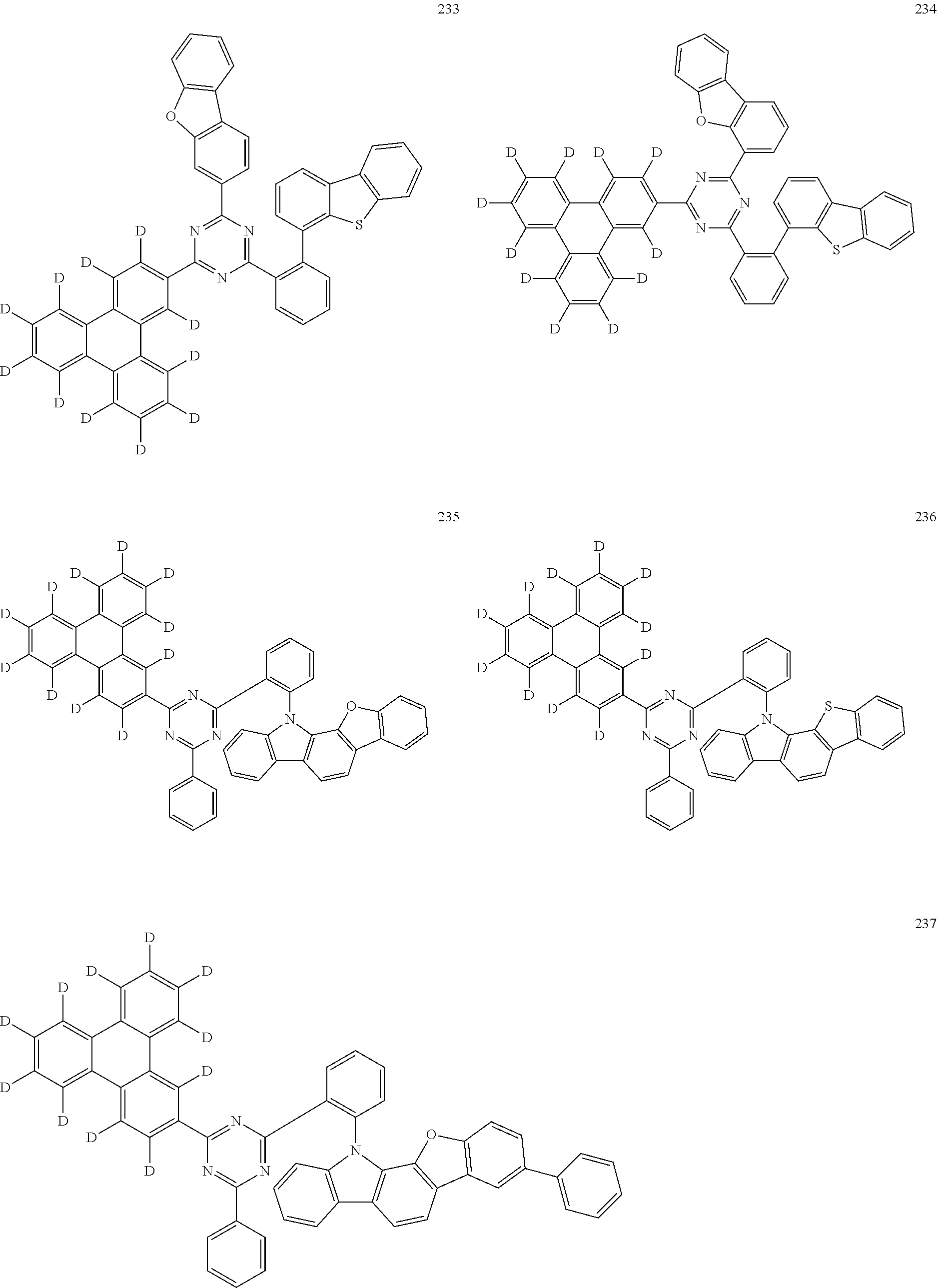

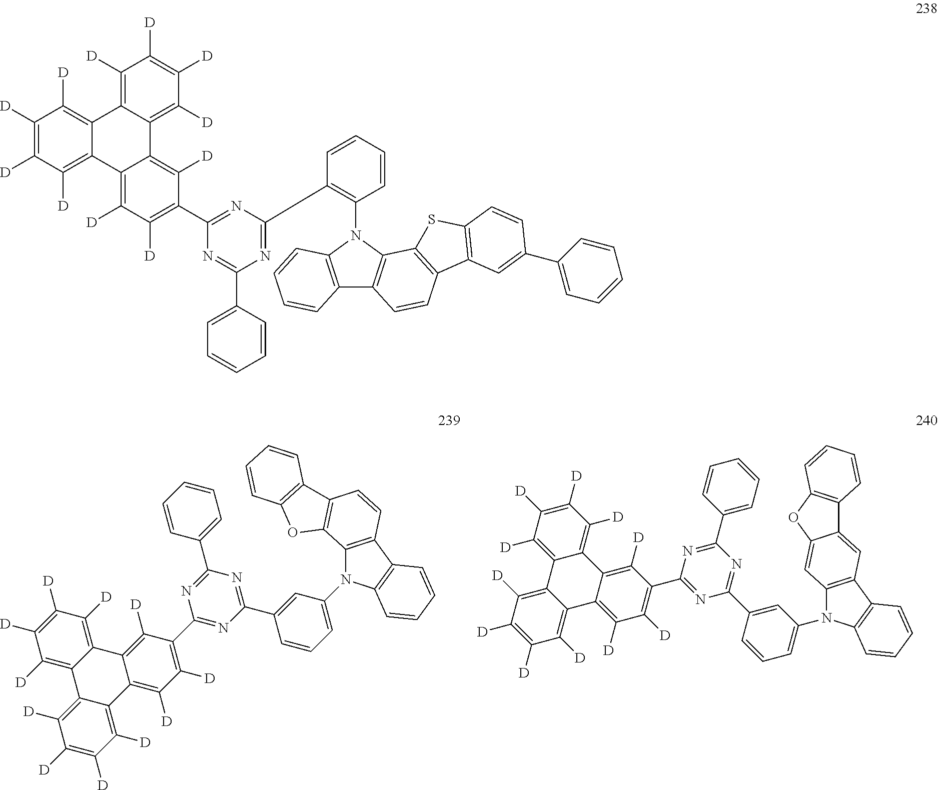 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7cc(-c8ccccc8)ccc7sc6c54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8oc7c65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7c(cc65)oc5ccccc57)c4)n3)c([2H])c([2H])c21]

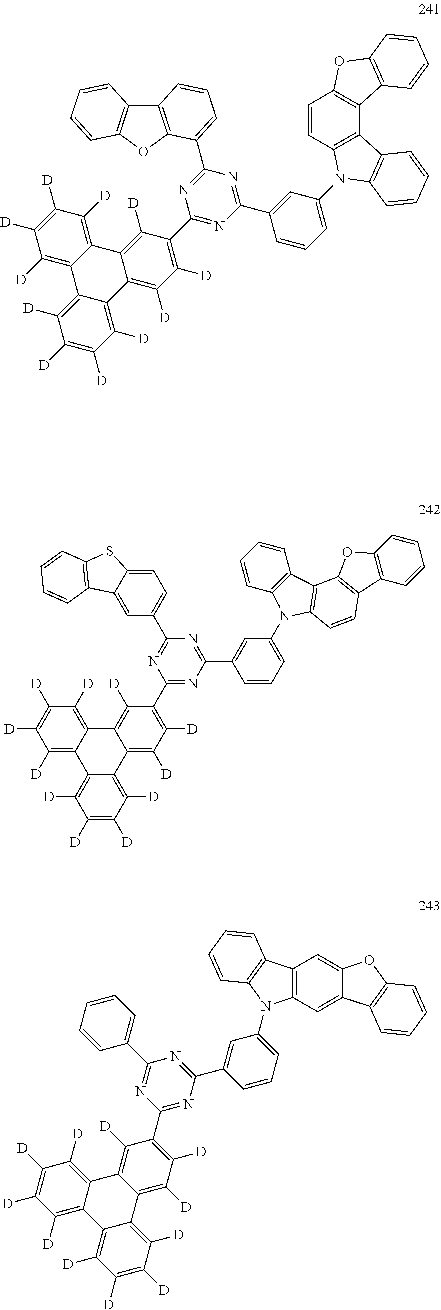 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6c7c(ccc65)oc5ccccc57)c4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6c7oc8ccccc8c7ccc65)c4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7oc8ccccc8c7cc65)c4)n3)c([2H])c([2H])c21]

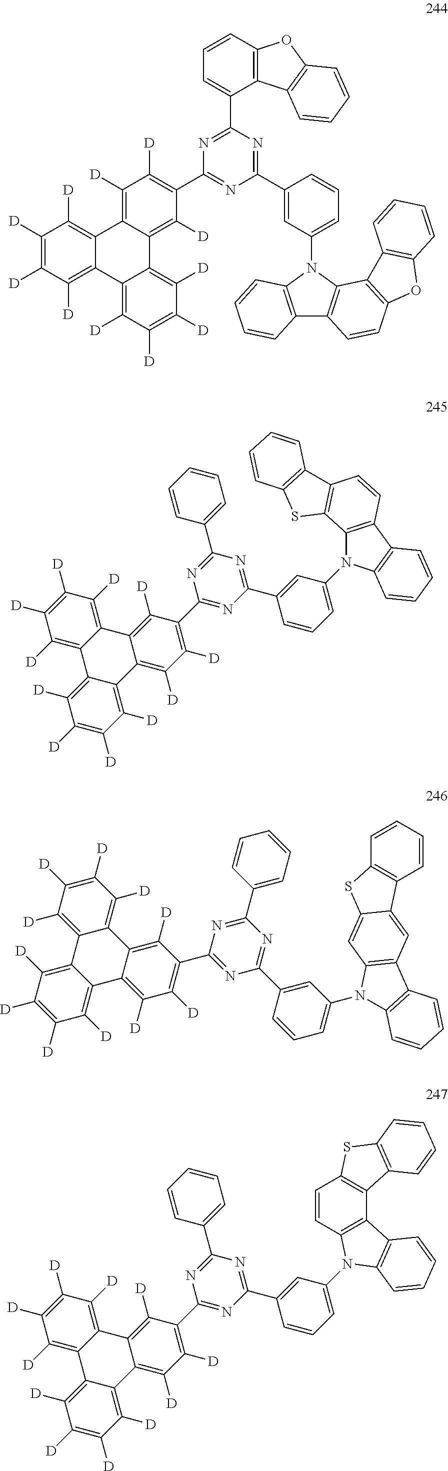

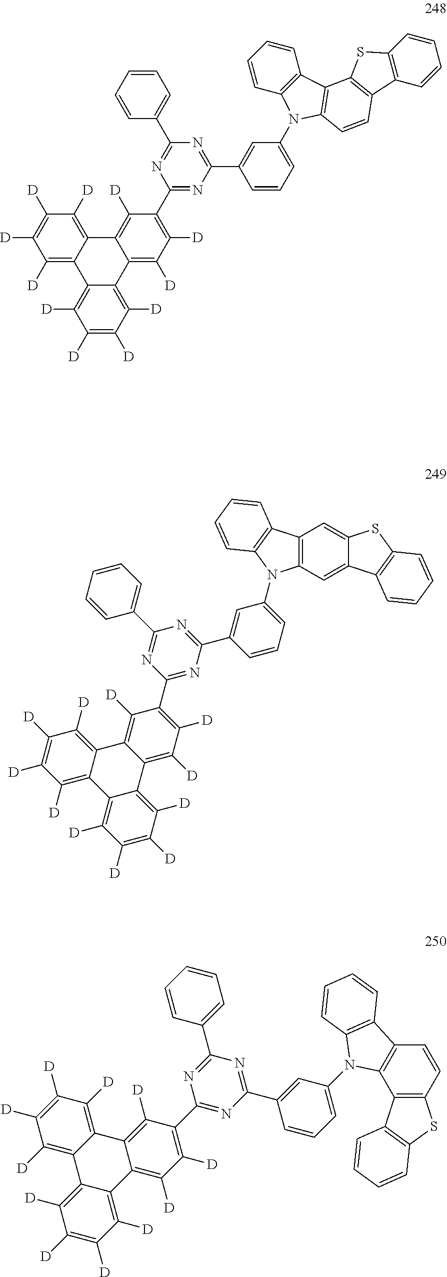 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6c7sc8ccccc8c7ccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7sc8ccccc8c7cc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7sc8ccccc8c7c65)c4)n3)c([2H])c([2H])c21]

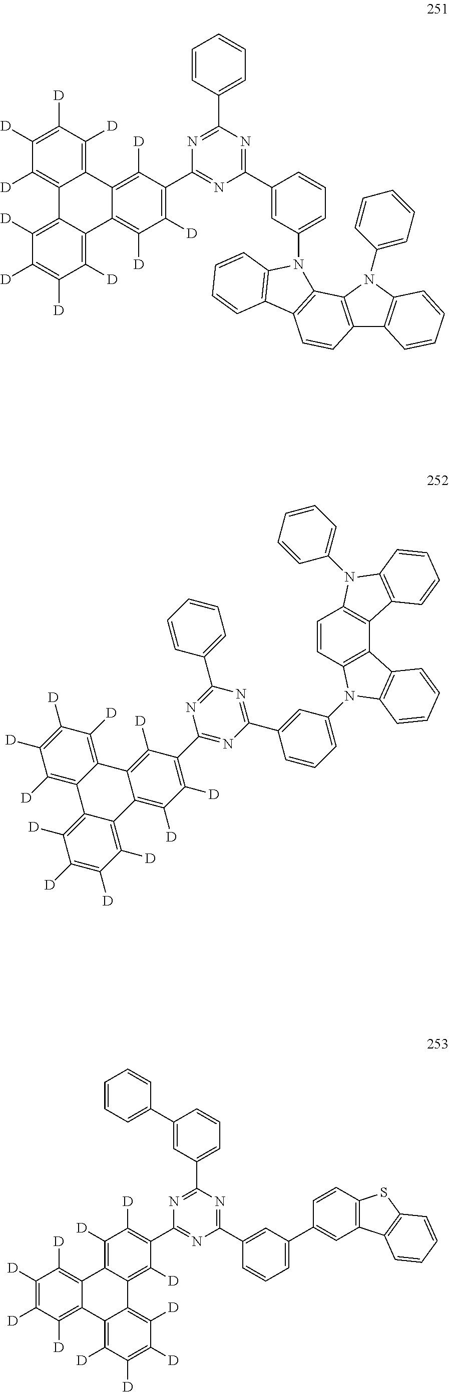 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8n(-c8ccccc8)c7c65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6c7c8ccccc8n(-c8ccccc8)c7ccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6sc7ccccc7c6c5)c4)n3)c([2H])c([2H])c21]

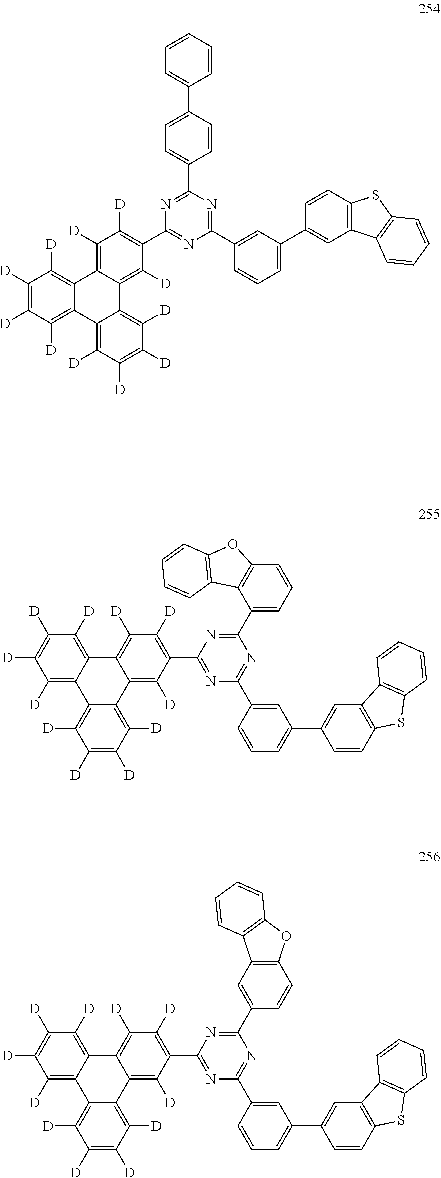 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6sc7ccccc7c6c5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4ccc5oc6ccccc6c5c4)n3)c([2H])c([2H])c21]

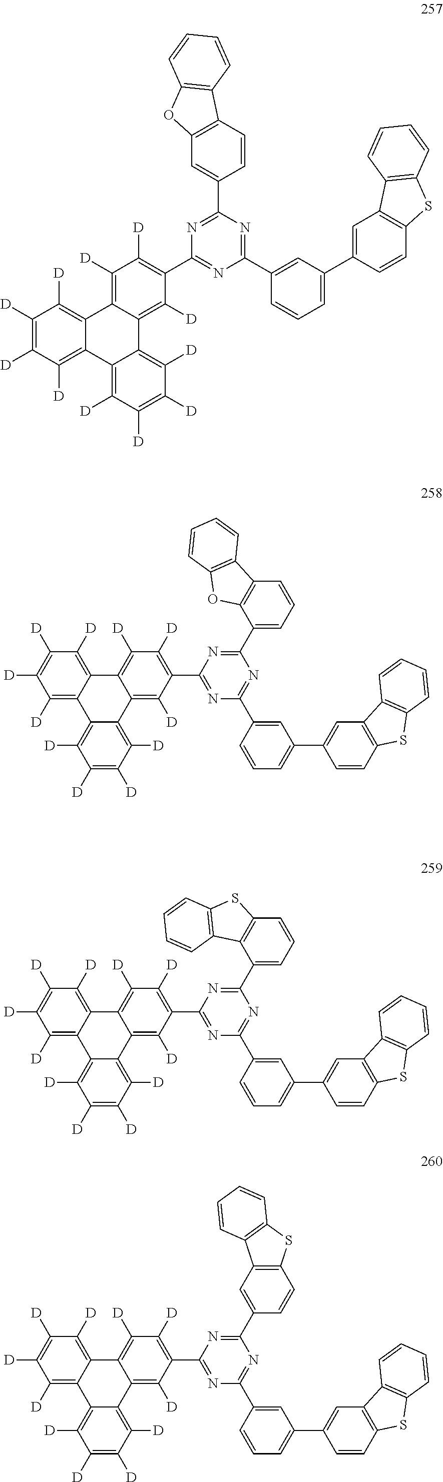

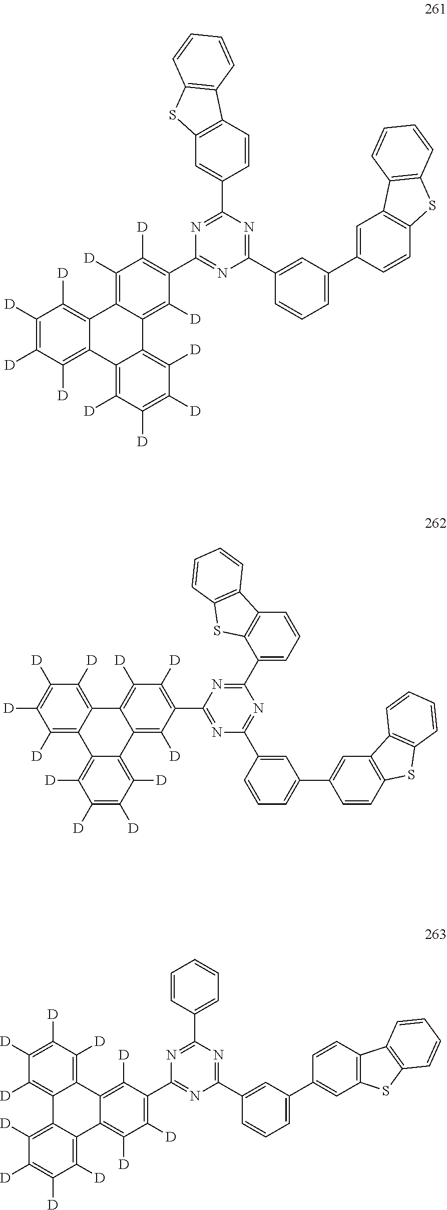 [SMILES: [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6c(c5)sc5ccccc56)c4)n3)c([2H])c([2H])c21]

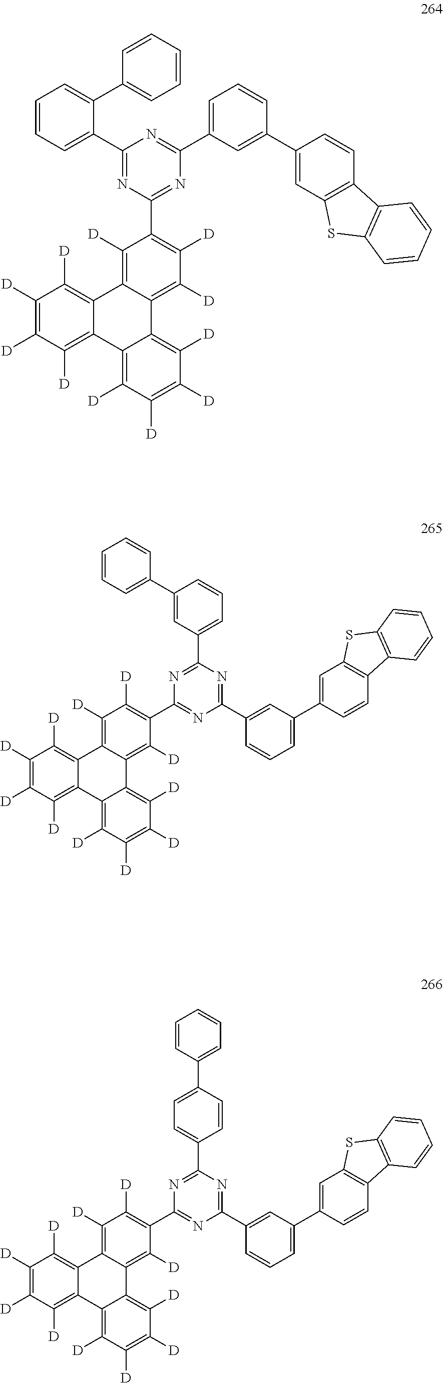 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6c(c5)sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6c(c5)sc5ccccc56)c4)n3)c([2H])c([2H])c21]

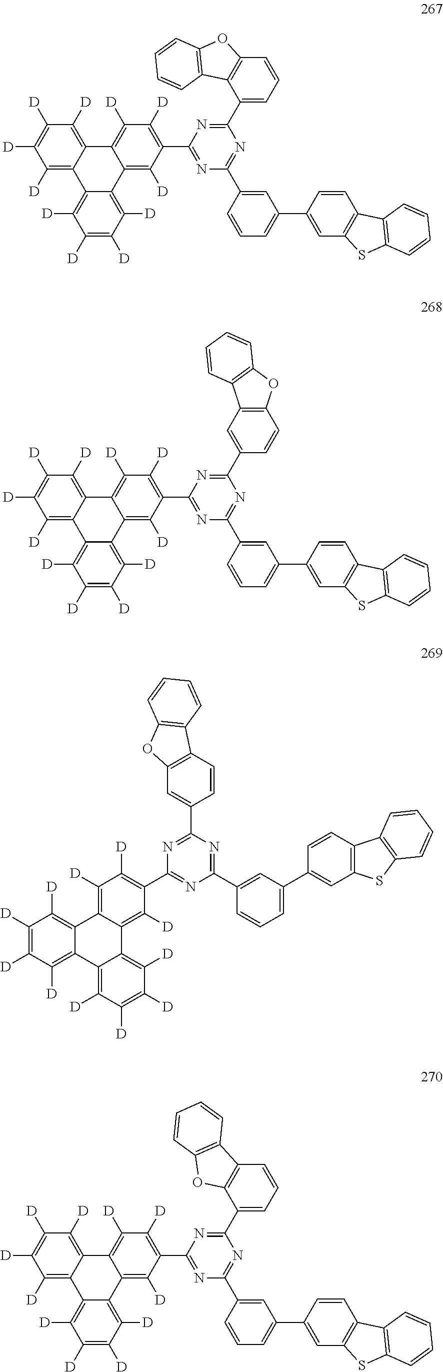

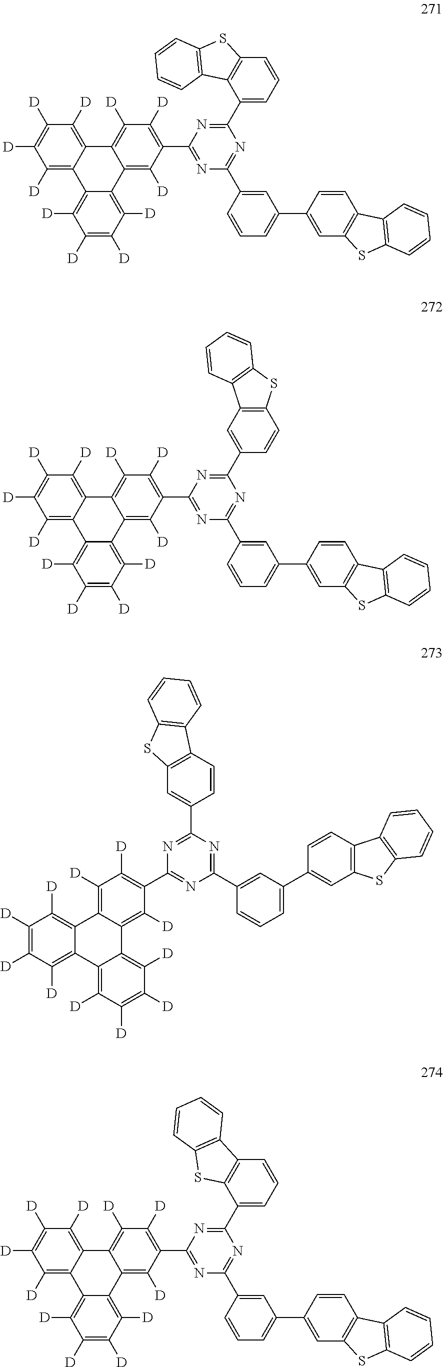

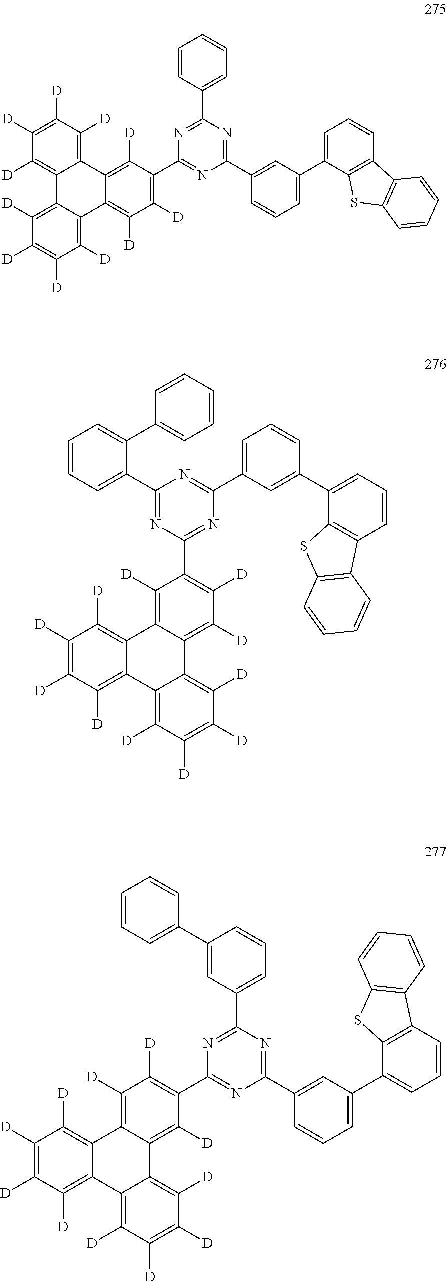 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c5sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c5sc5ccccc56)c4)n3)c([2H])c([2H])c21]

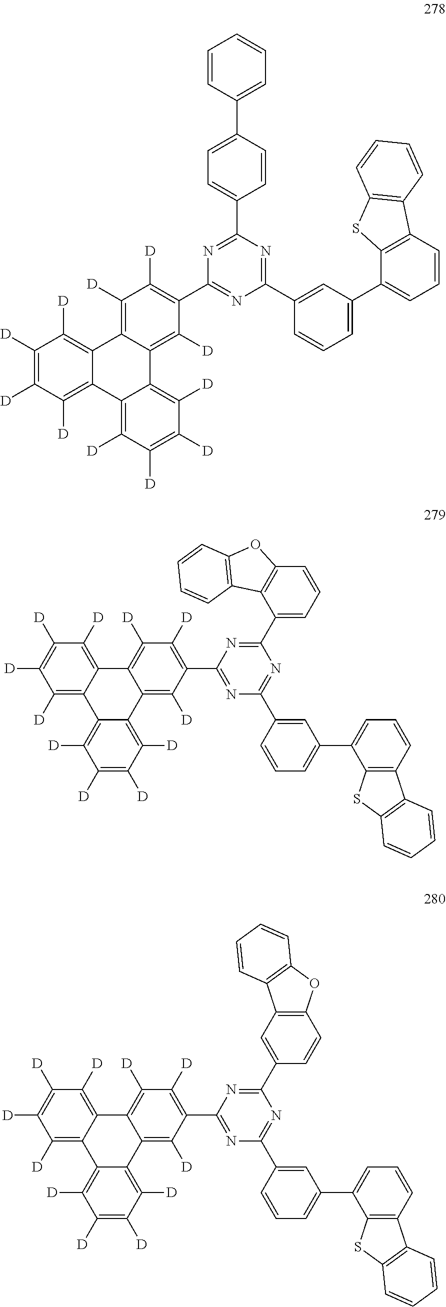 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5cccc6c5sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5oc6ccccc6c5c4)n3)c([2H])c([2H])c21]

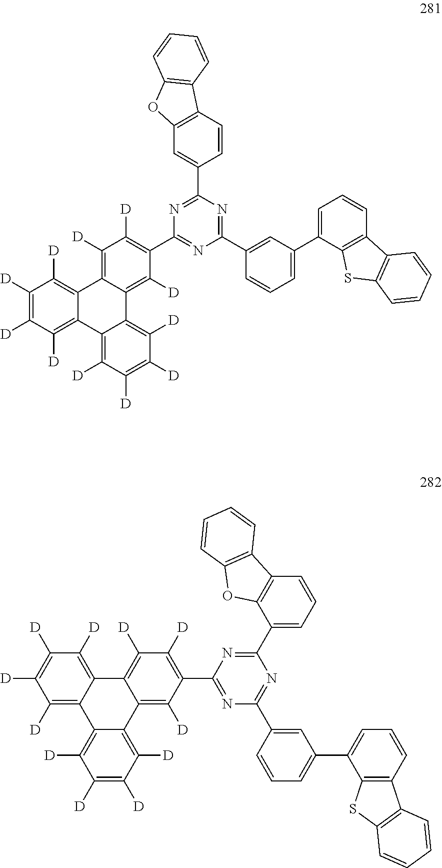 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5c4oc4ccccc45)n3)c([2H])c([2H])c21]

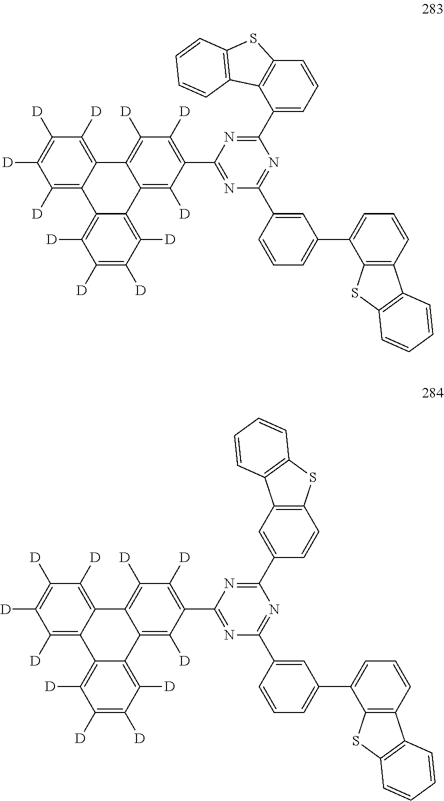 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5sc6ccccc6c5c4)n3)c([2H])c([2H])c21]

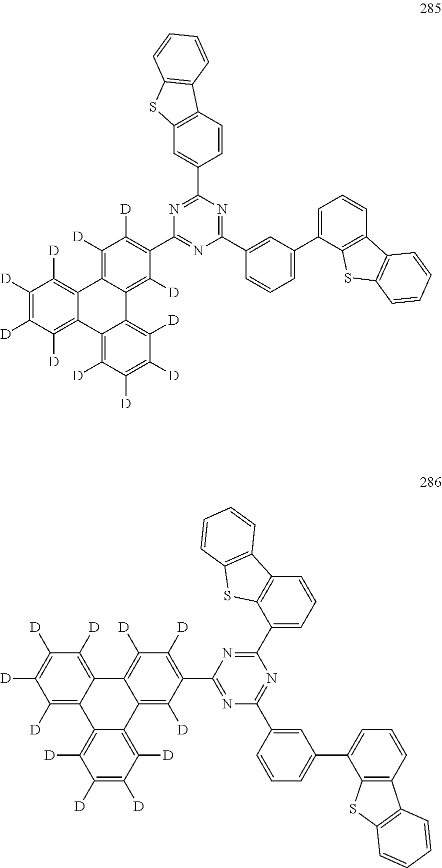 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5c4sc4ccccc45)n3)c([2H])c([2H])c21]

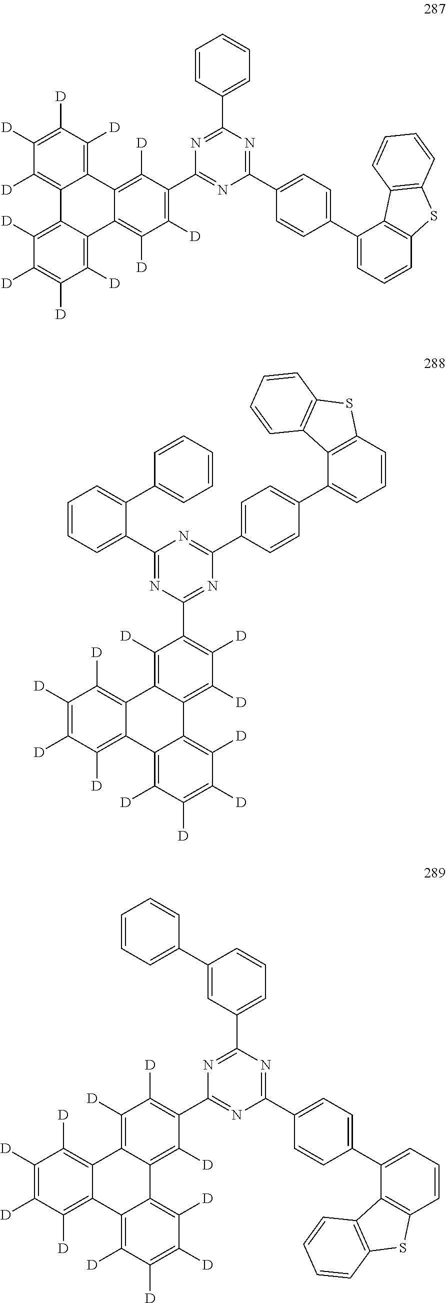 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6sc7ccccc7c56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc(-c5ccccc5)c4)n3)c([2H])c([2H])c21]

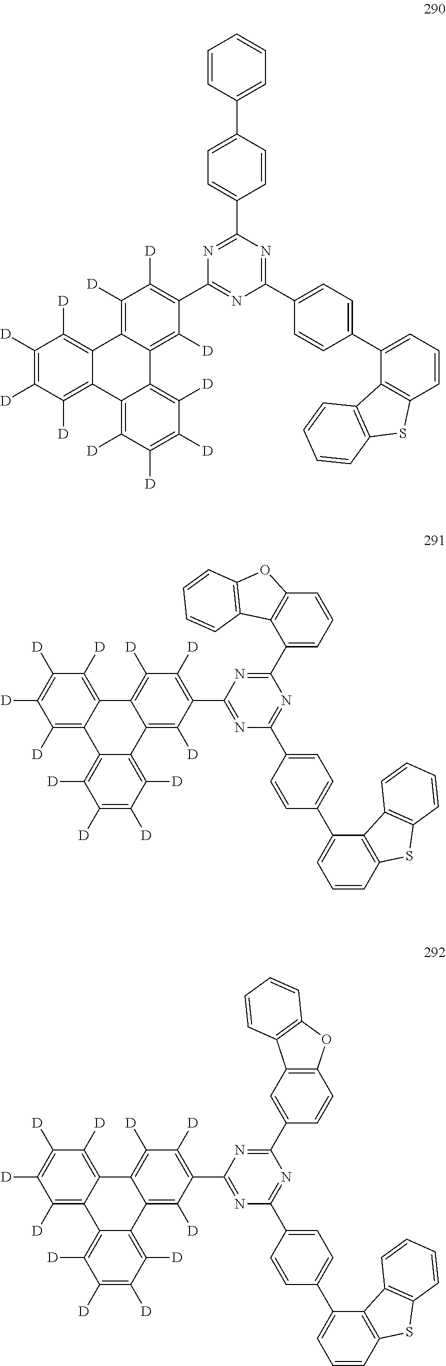 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6sc7ccccc7c56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5oc6ccccc6c5c4)n3)c([2H])c([2H])c21]

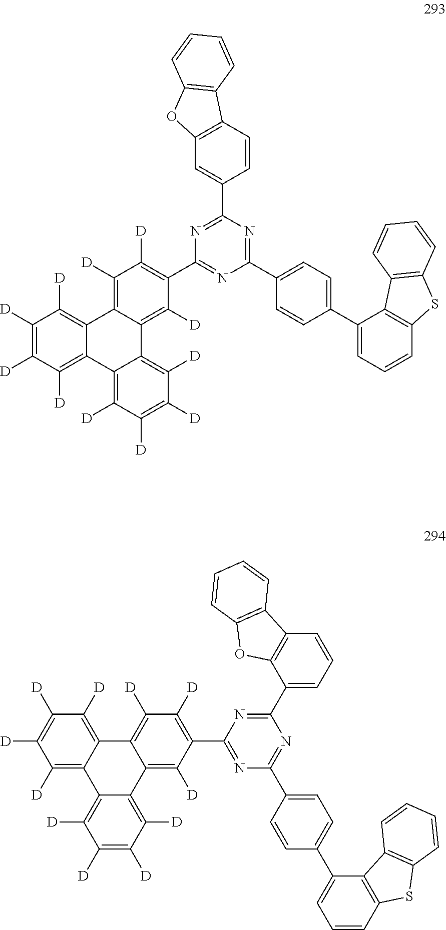 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5c4oc4ccccc45)n3)c([2H])c([2H])c21]

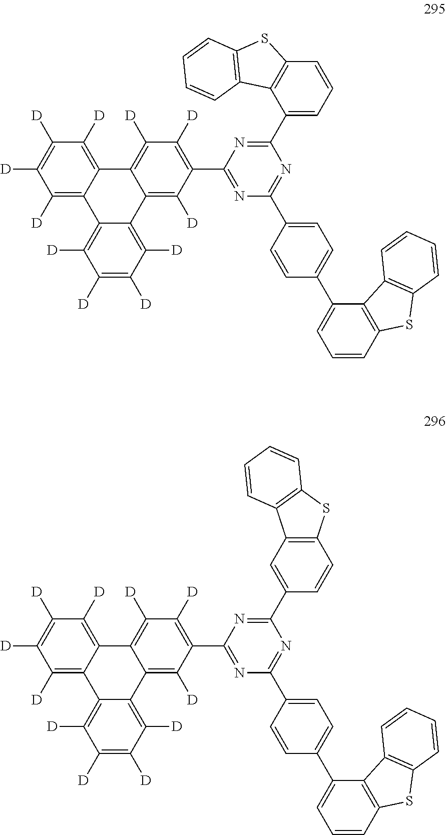 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5sc6ccccc6c5c4)n3)c([2H])c([2H])c21]

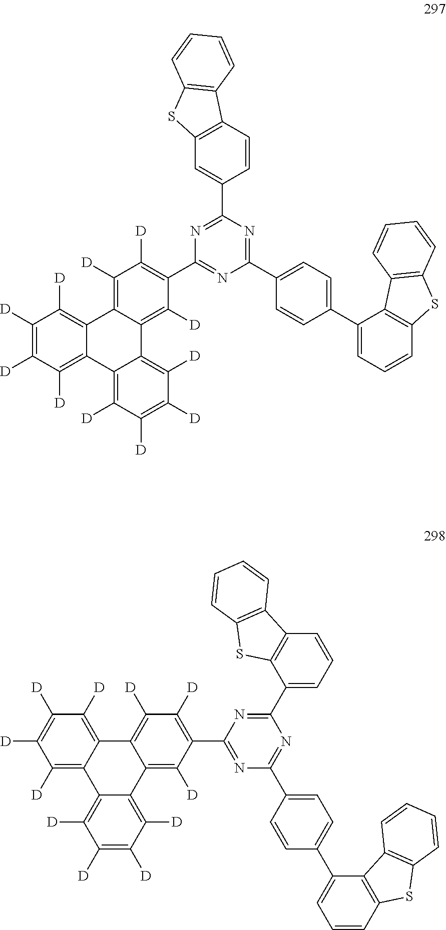 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5c4sc4ccccc45)n3)c([2H])c([2H])c21]

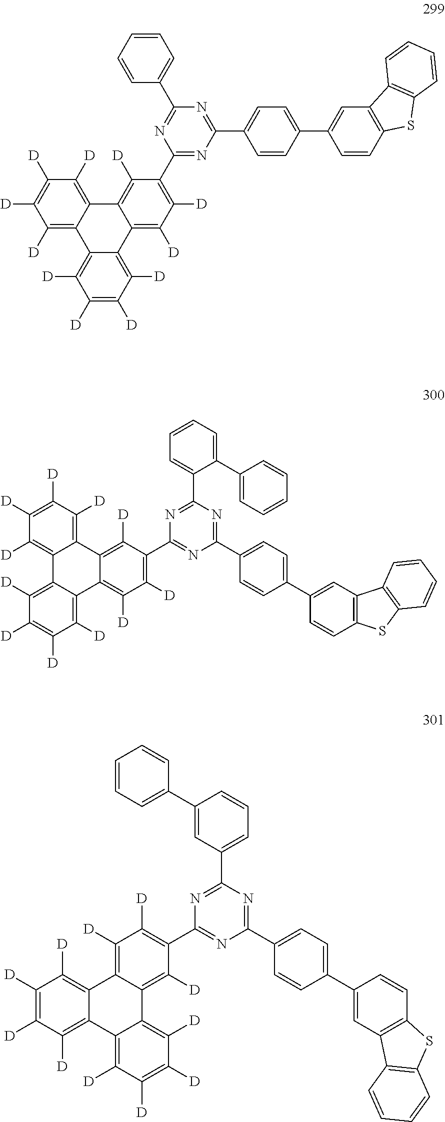 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5ccc6sc7ccccc7c6c5)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc(-c5ccccc5)c4)n3)c([2H])c([2H])c21]

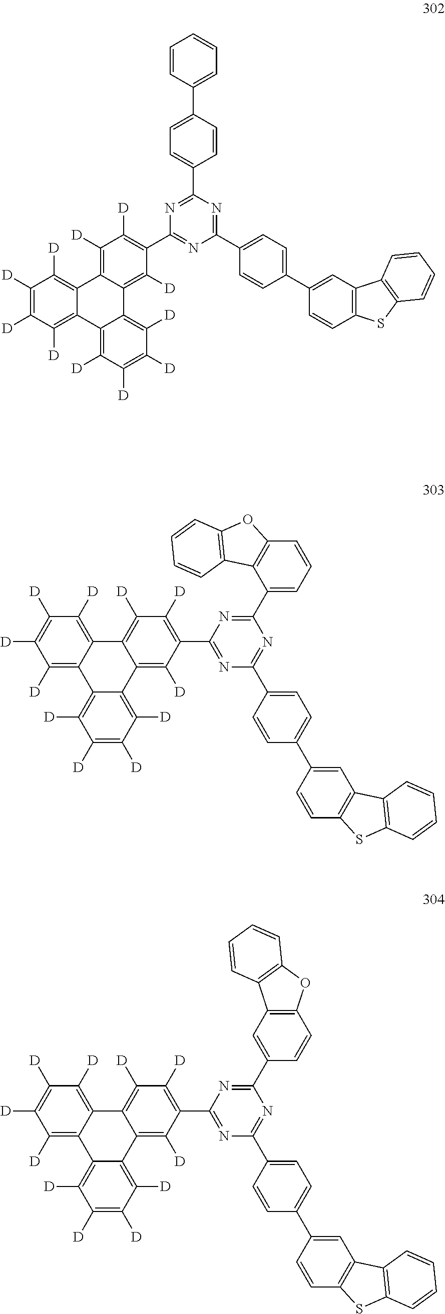 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5ccc6sc7ccccc7c6c5)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5oc6ccccc6c5c4)n3)c([2H])c([2H])c21]

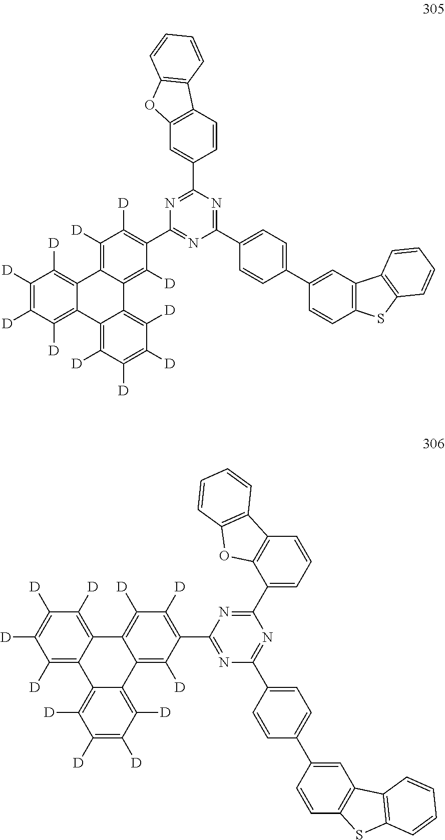 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5c4oc4ccccc45)n3)c([2H])c([2H])c21]

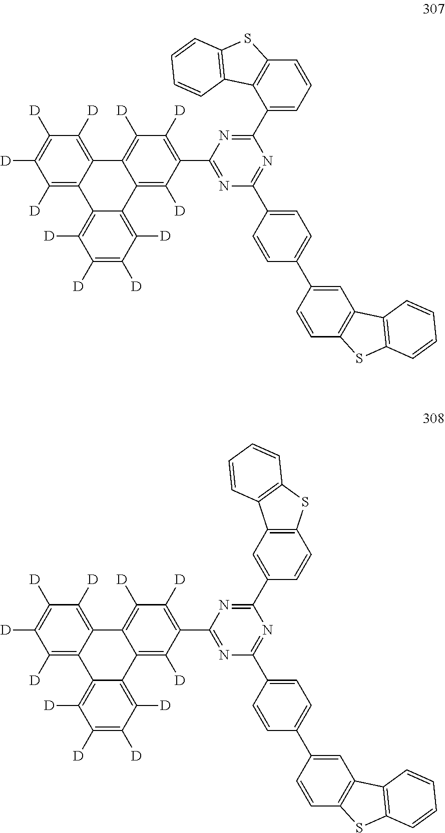 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5sc6ccccc6c5c4)n3)c([2H])c([2H])c21]

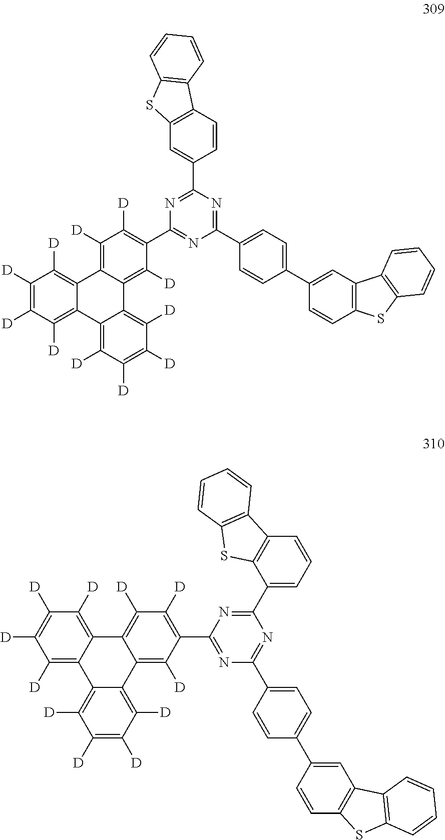 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5c4sc4ccccc45)n3)c([2H])c([2H])c21]

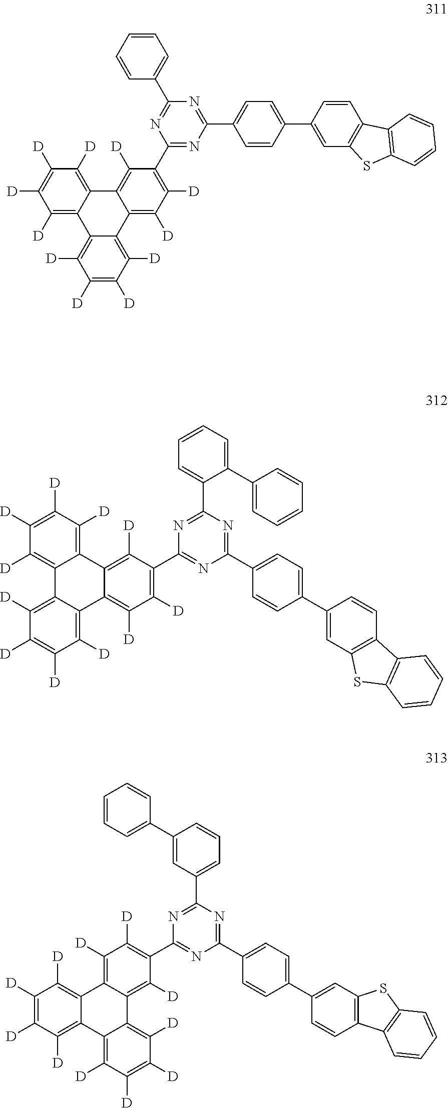 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5ccc6c(c5)sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc(-c5ccccc5)c4)n3)c([2H])c([2H])c21]

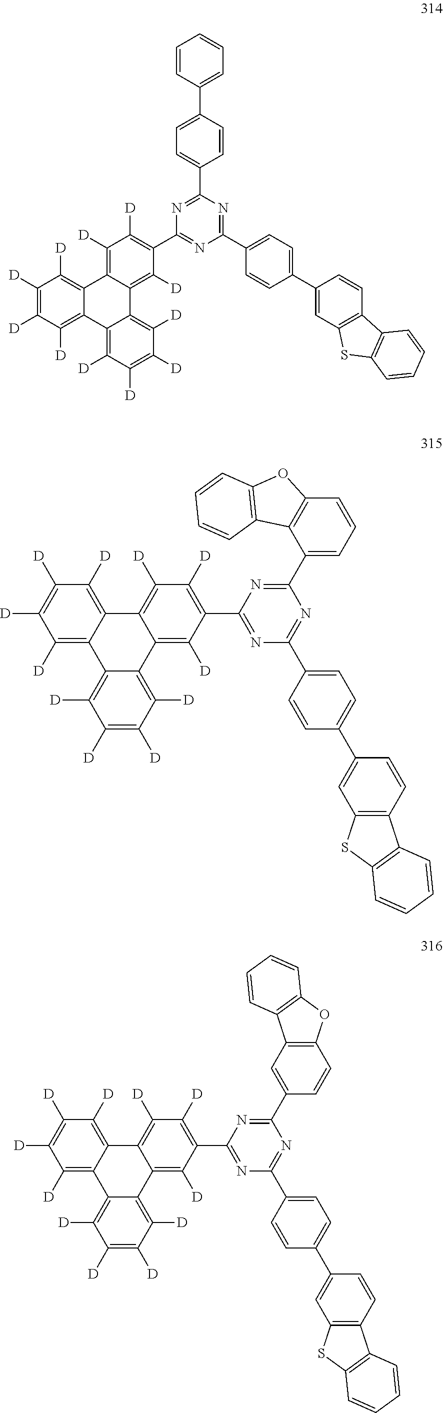 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5ccc6c(c5)sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5oc6ccccc6c5c4)n3)c([2H])c([2H])c21]

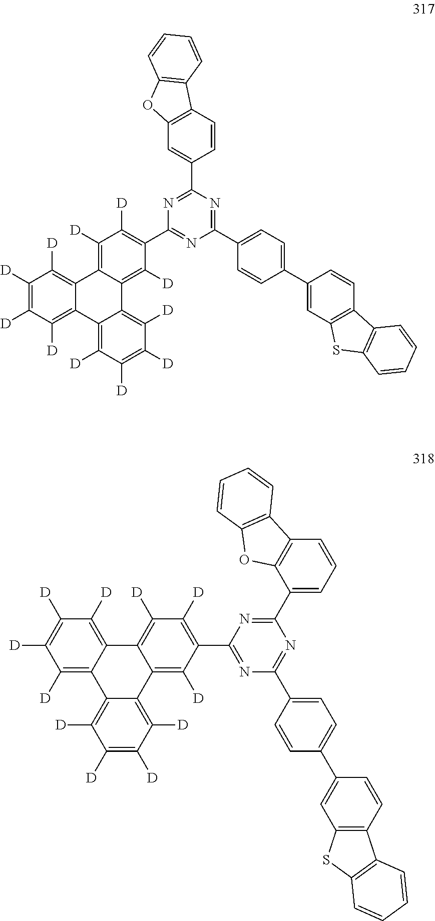 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4oc4ccccc45)n3)c([2H])c([2H])c21]

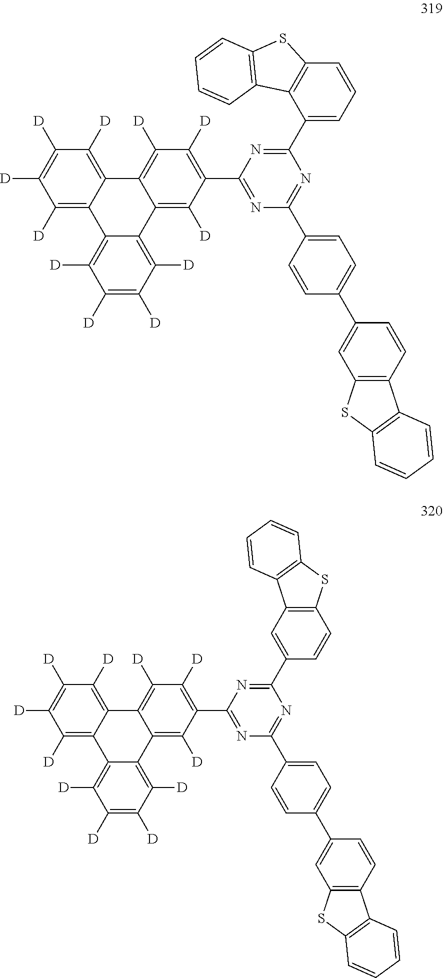 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5sc6ccccc6c5c4)n3)c([2H])c([2H])c21]

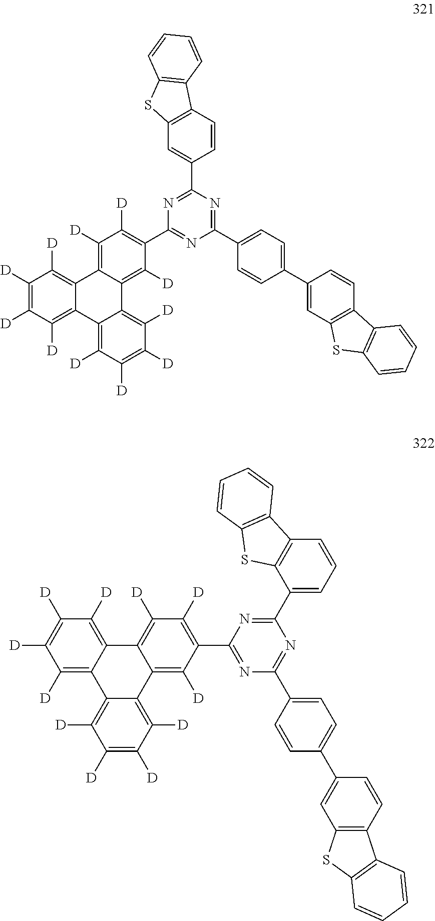 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4sc4ccccc45)n3)c([2H])c([2H])c21]

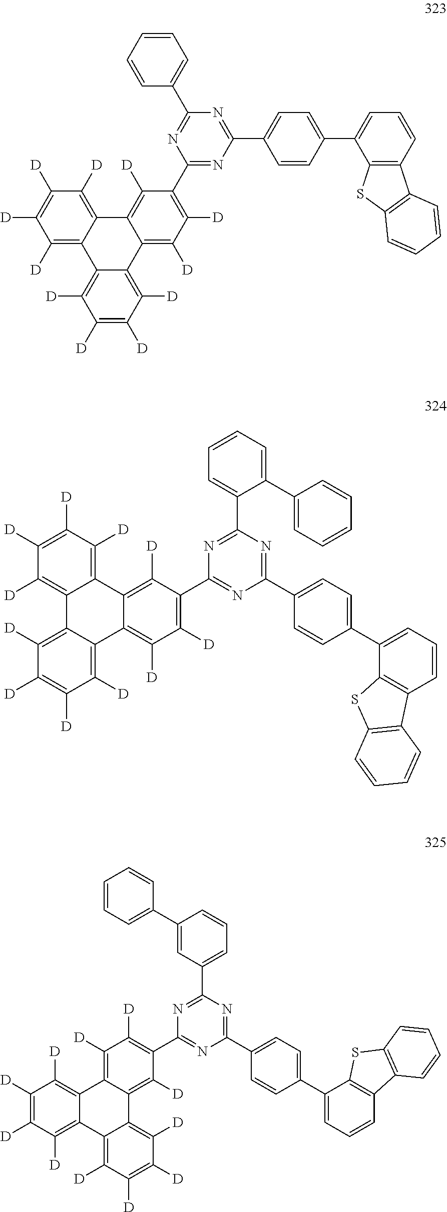 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc(-c5ccccc5)c4)n3)c([2H])c([2H])c21]

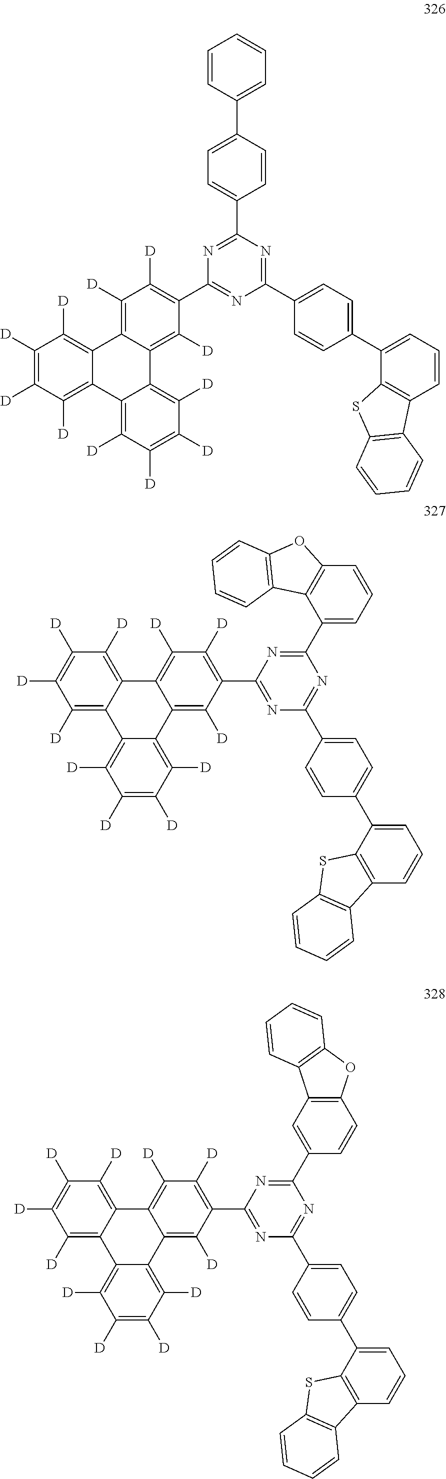 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6c5sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5oc6ccccc6c5c4)n3)c([2H])c([2H])c21]

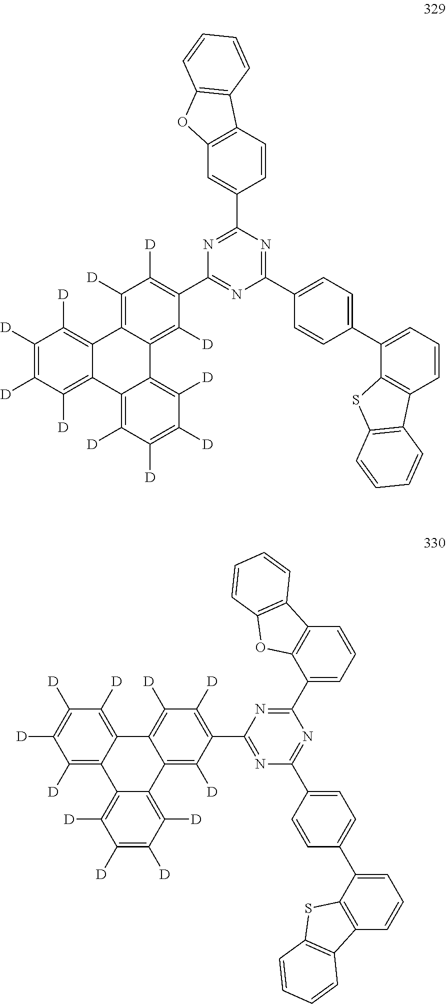 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4oc4ccccc45)n3)c([2H])c([2H])c21]

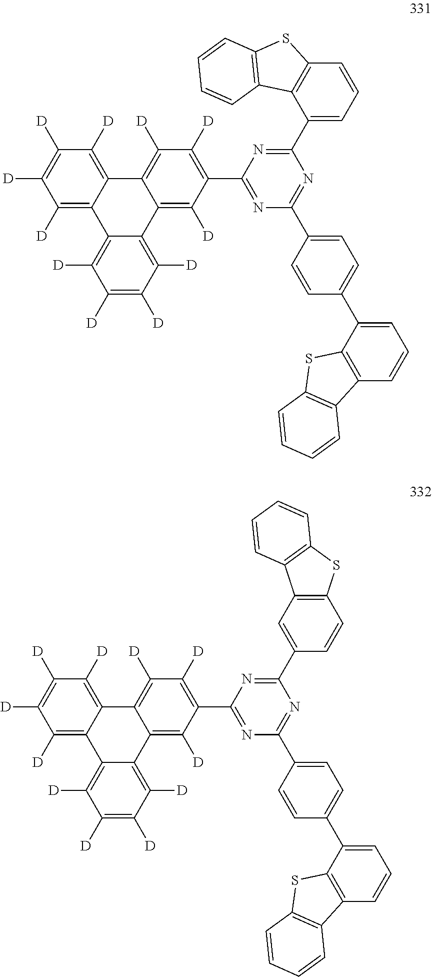 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5sc6ccccc6c5c4)n3)c([2H])c([2H])c21]

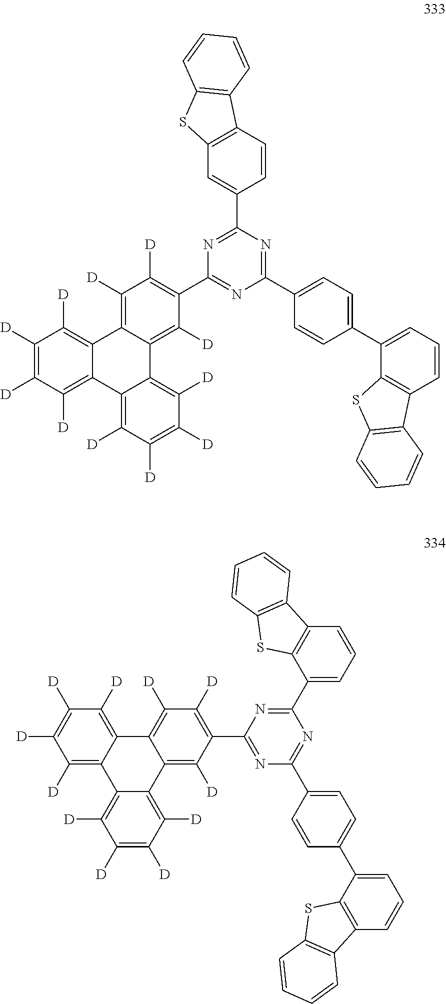 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4sc4ccccc45)n3)c([2H])c([2H])c21]

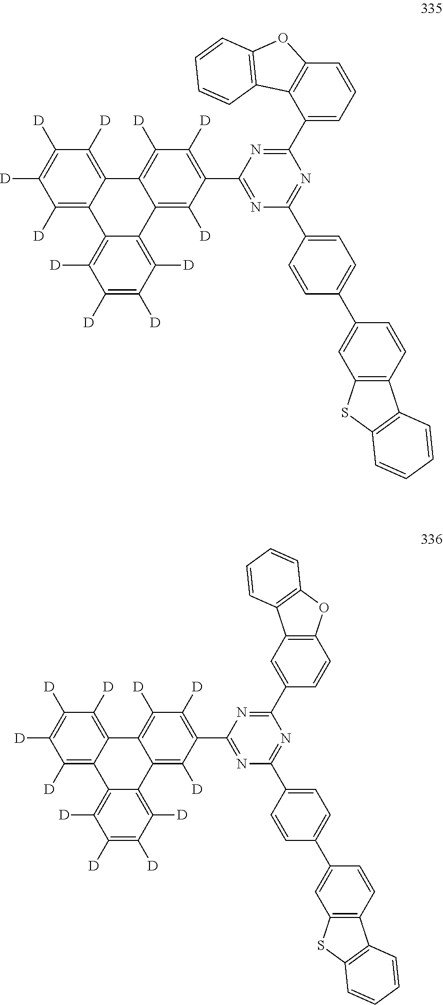 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5oc6ccccc6c5c4)n3)c([2H])c([2H])c21]

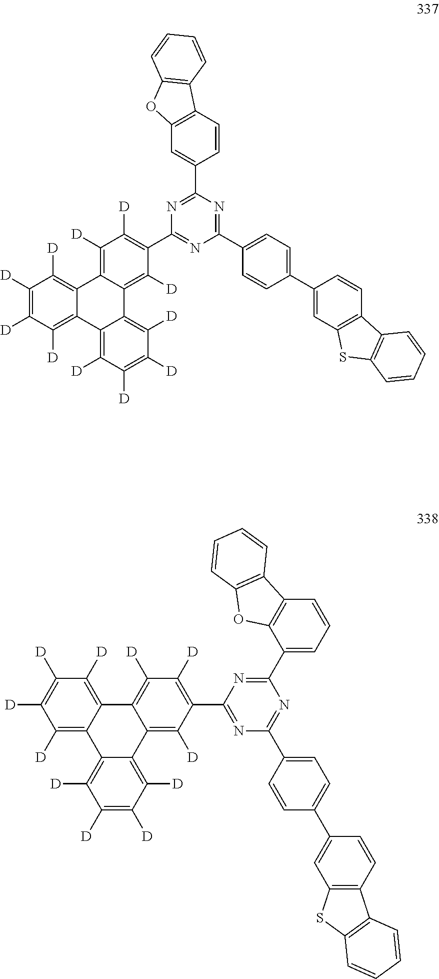 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4oc4ccccc45)n3)c([2H])c([2H])c21]

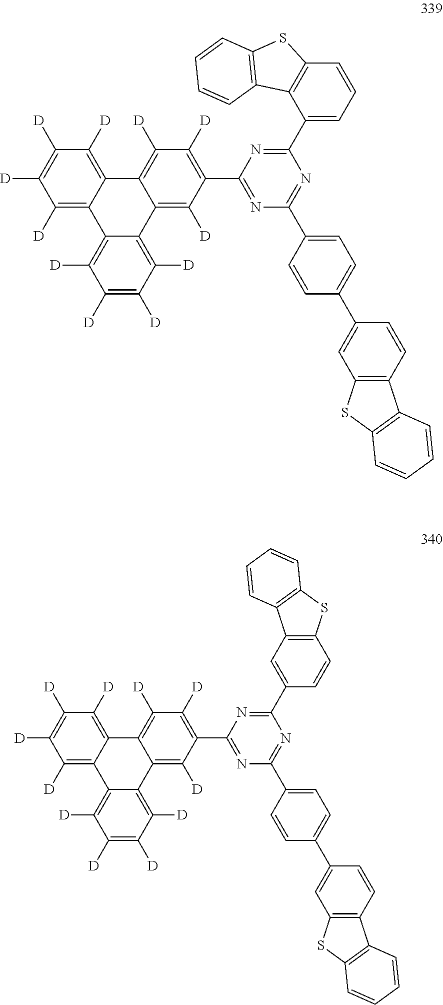 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5sc6ccccc6c5c4)n3)c([2H])c([2H])c21]

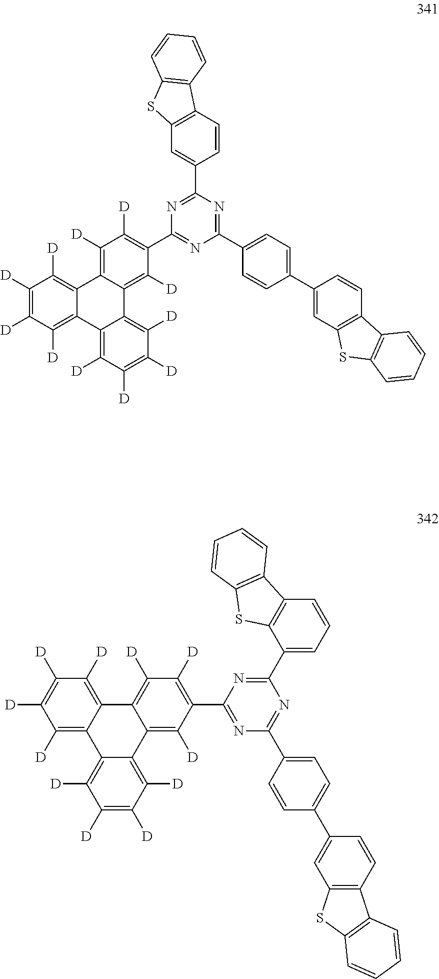 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4sc4ccccc45)n3)c([2H])c([2H])c21]

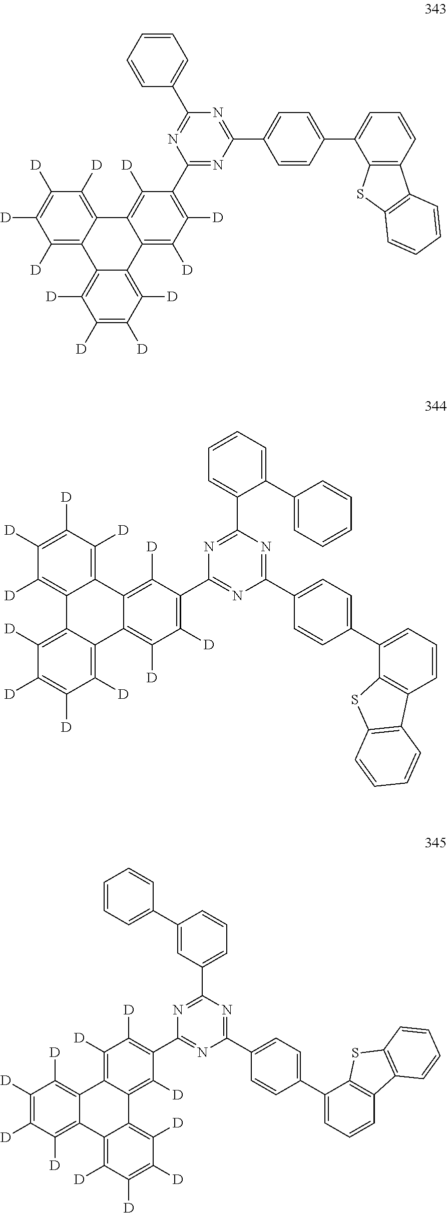 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc(-c5ccccc5)c4)n3)c([2H])c([2H])c21]

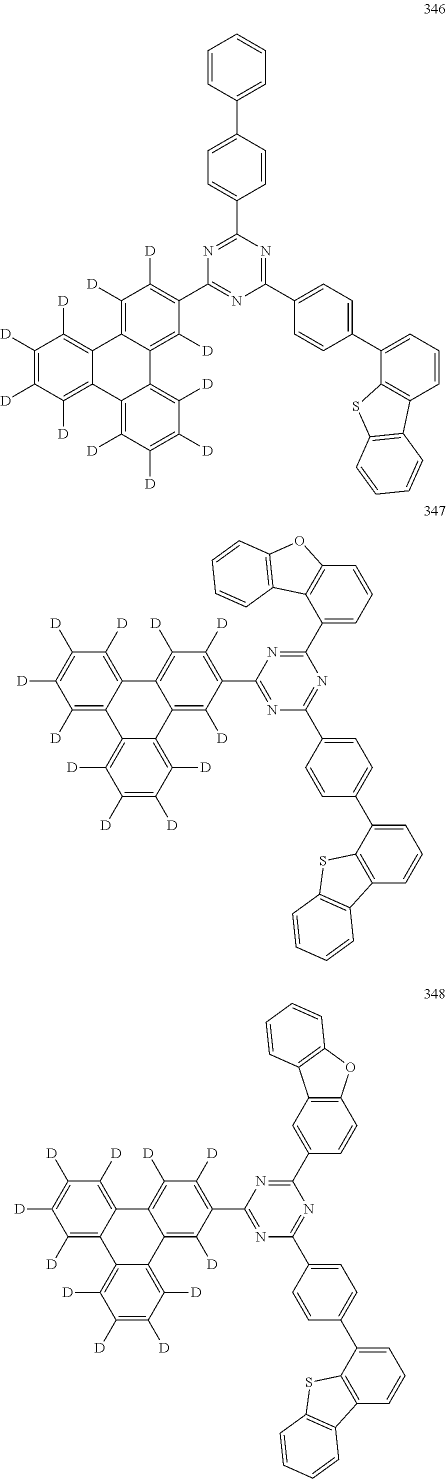 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6c5sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5oc6ccccc6c5c4)n3)c([2H])c([2H])c21]

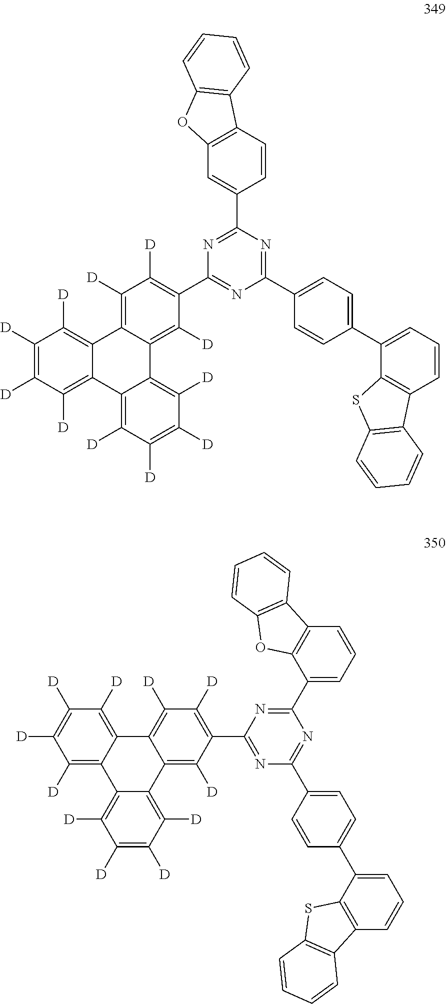 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4oc4ccccc45)n3)c([2H])c([2H])c21]

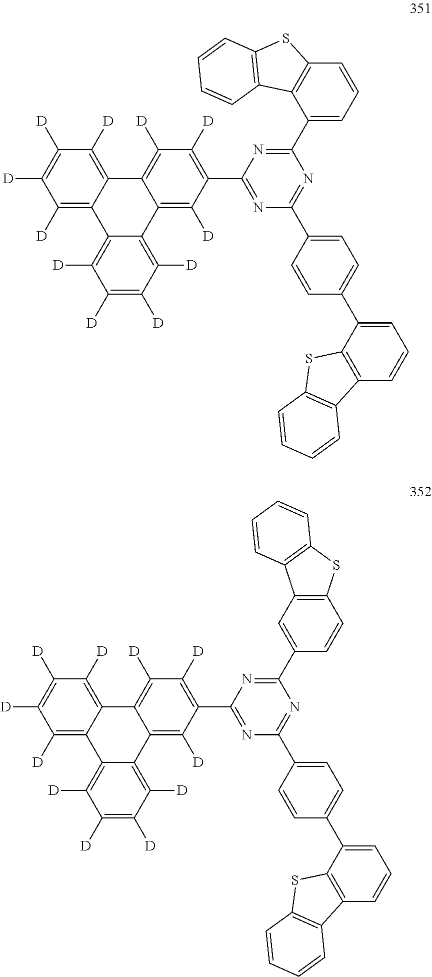 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5sc6ccccc6c5c4)n3)c([2H])c([2H])c21]

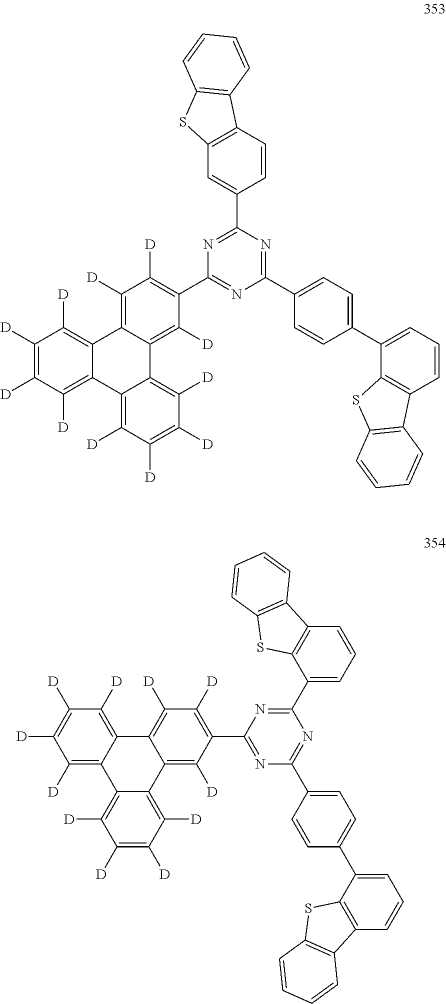 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4sc4ccccc45)n3)c([2H])c([2H])c21]

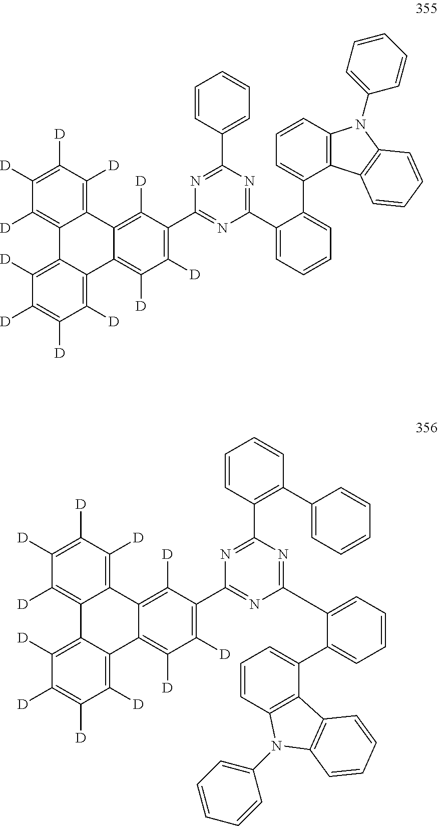 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c4c4ccccc4n5-c4ccccc4)n3)c([2H])c([2H])c21]

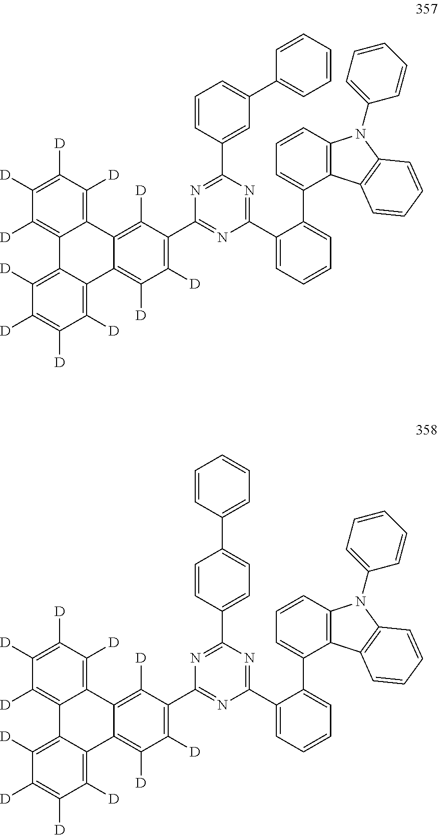 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c4c4ccccc4n5-c4ccccc4)n3)c([2H])c([2H])c21]

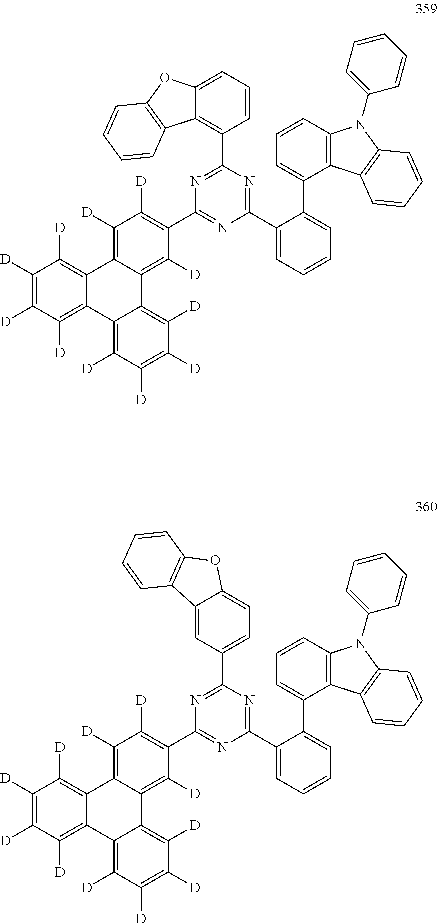 [SMILES: [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5c4c4ccccc4n5-c4ccccc4)n3)c([2H])c([2H])c21]

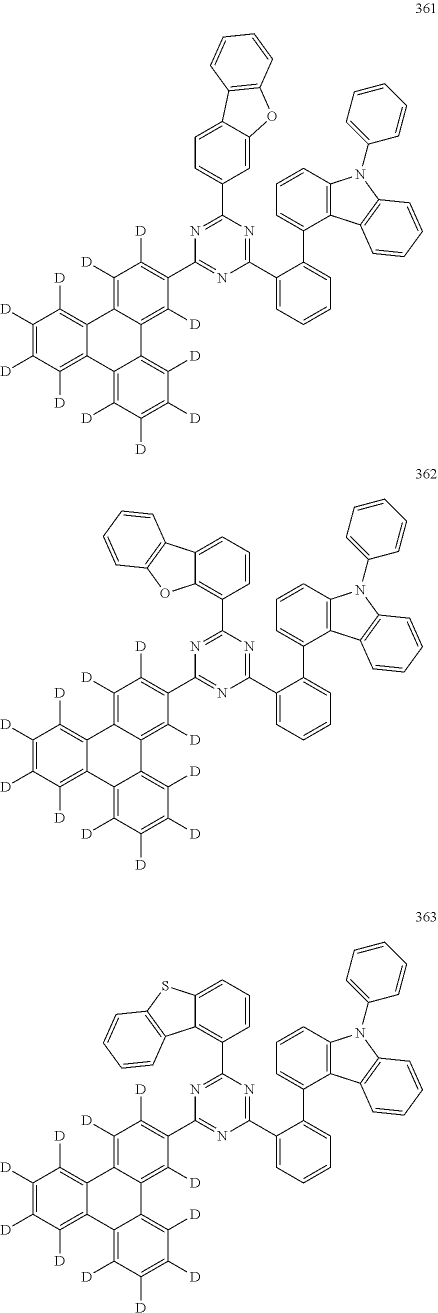 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5sc6ccccc6c45)n3)c([2H])c([2H])c21]

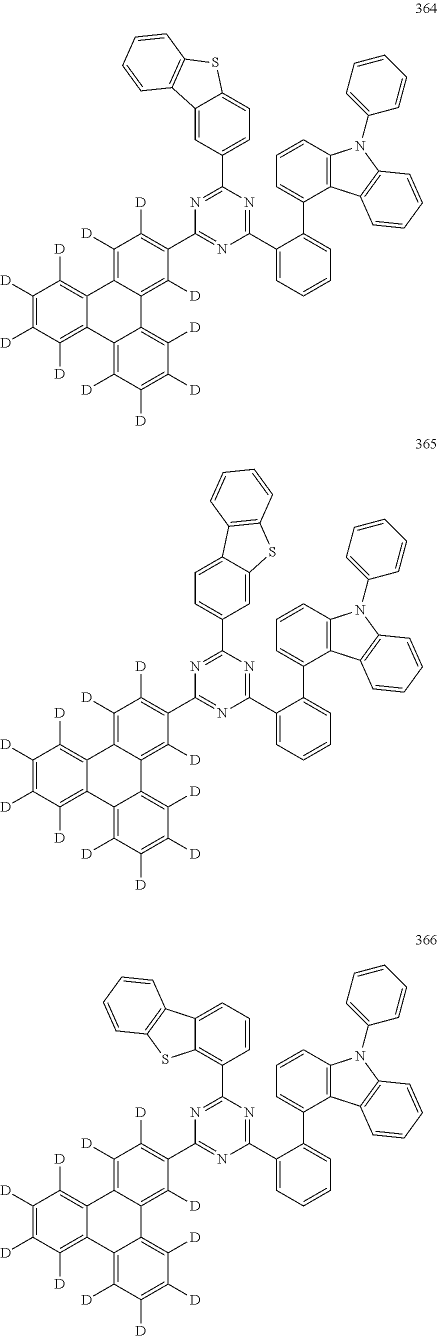 [SMILES: [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5c4sc4ccccc45)n3)c([2H])c([2H])c21]

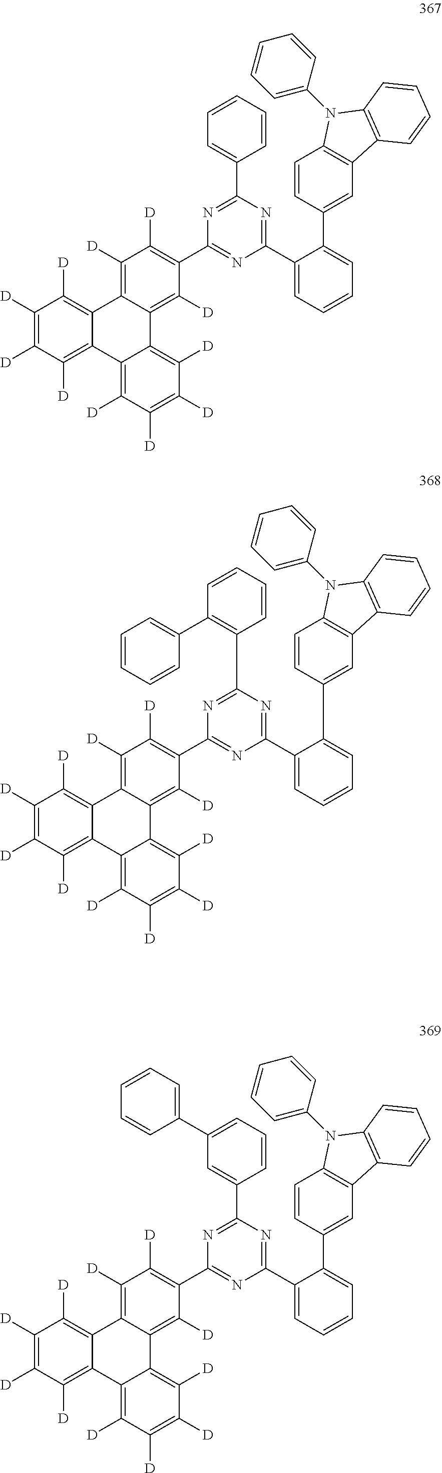 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5c(c4)c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c(c4)c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5c(c4)c4ccccc4n5-c4ccccc4)n3)c([2H])c([2H])c21]

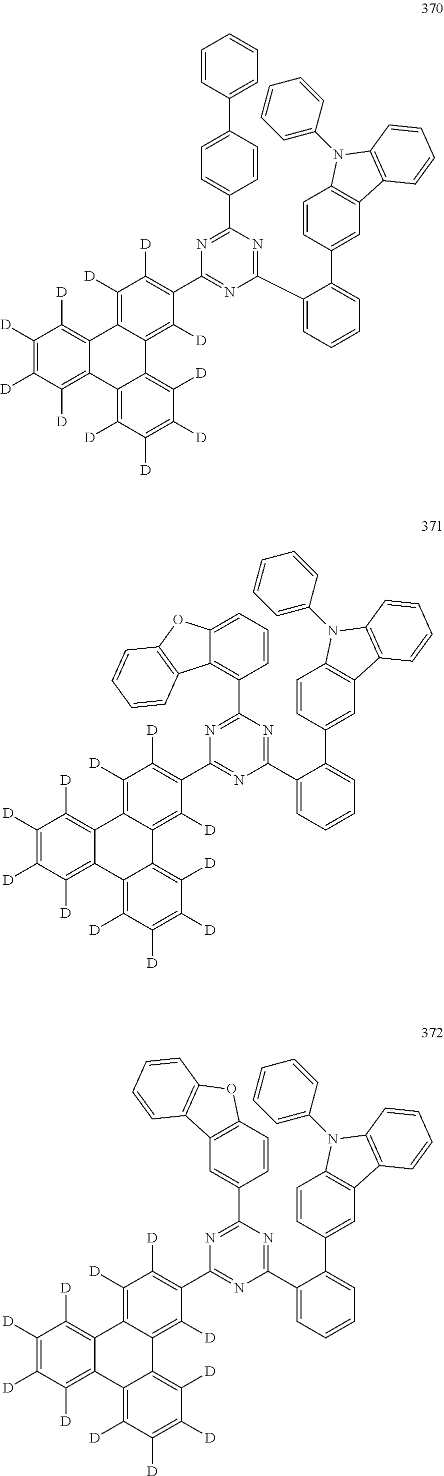

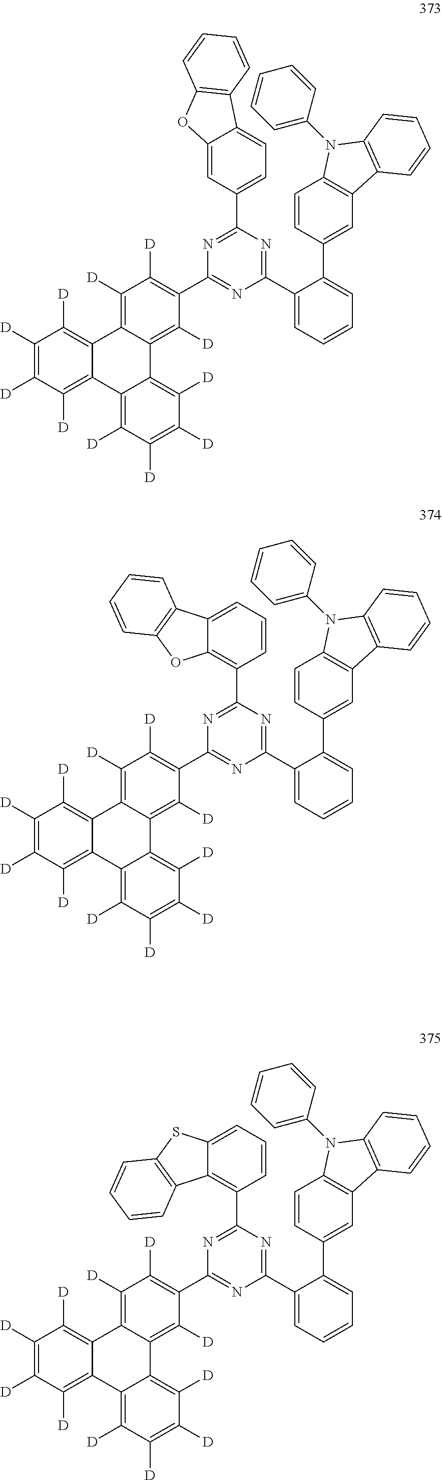

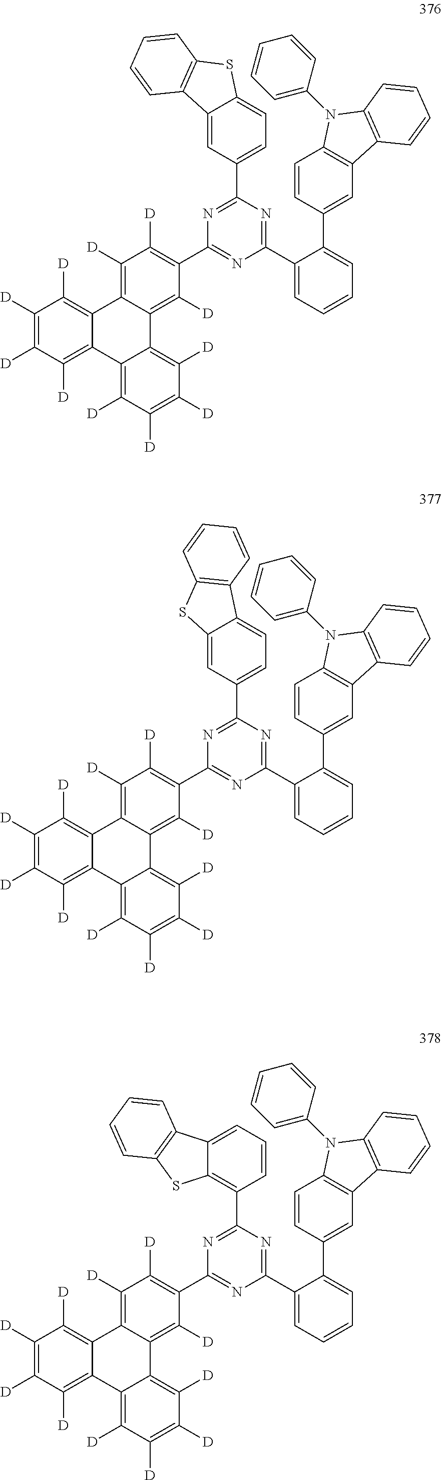

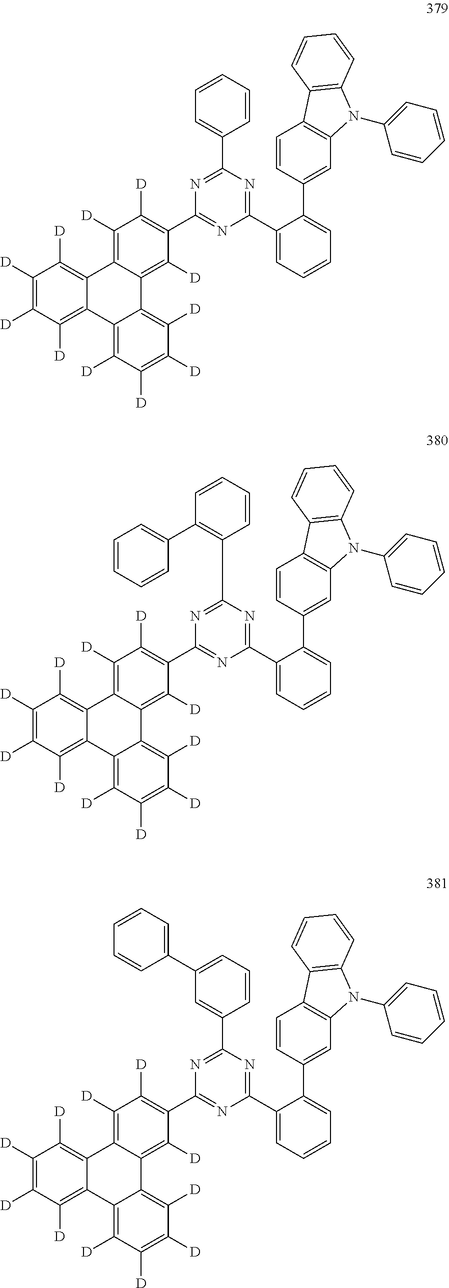 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5c6ccccc6n(-c6ccccc6)c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c6ccccc6n(-c6ccccc6)c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5c6ccccc6n(-c6ccccc6)c5c4)n3)c([2H])c([2H])c21]

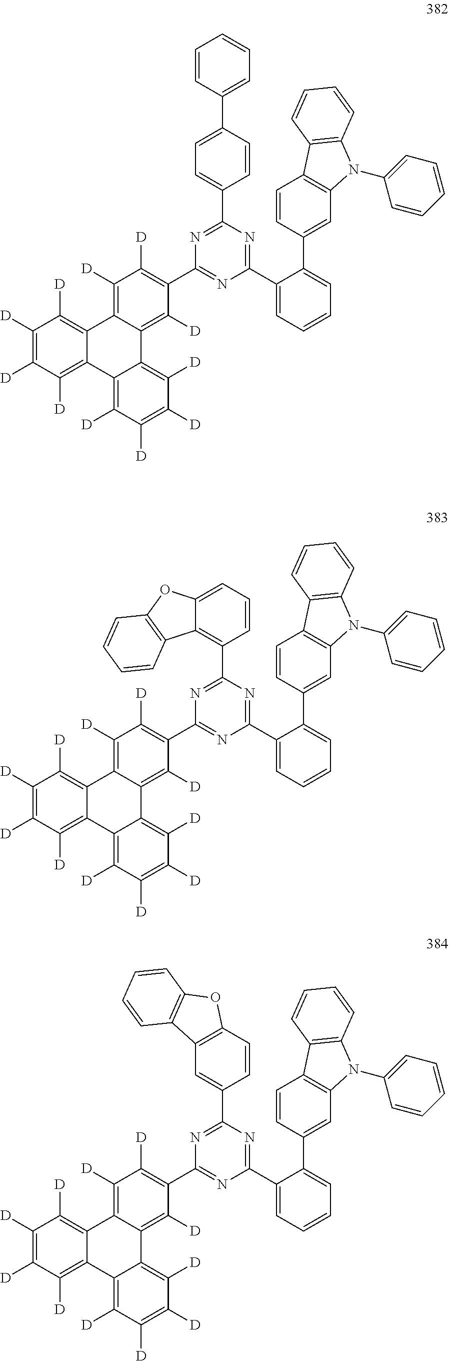

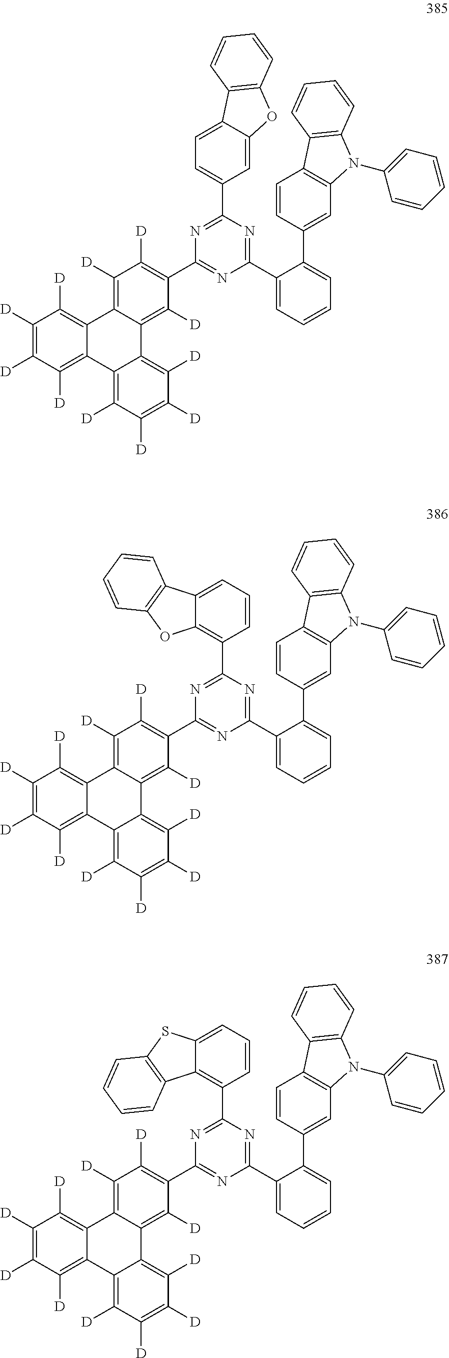

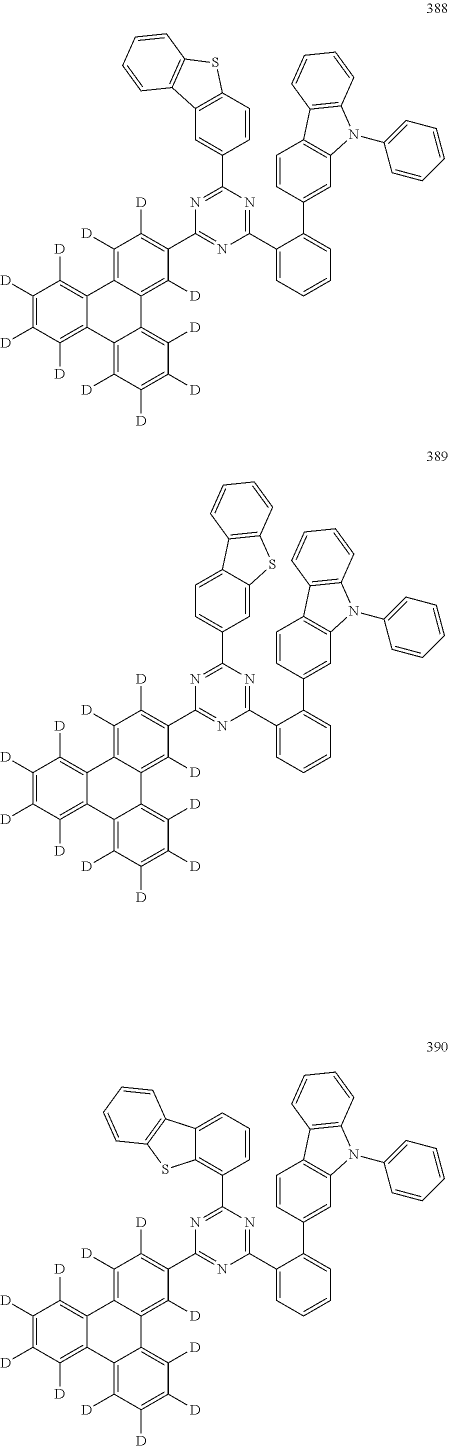

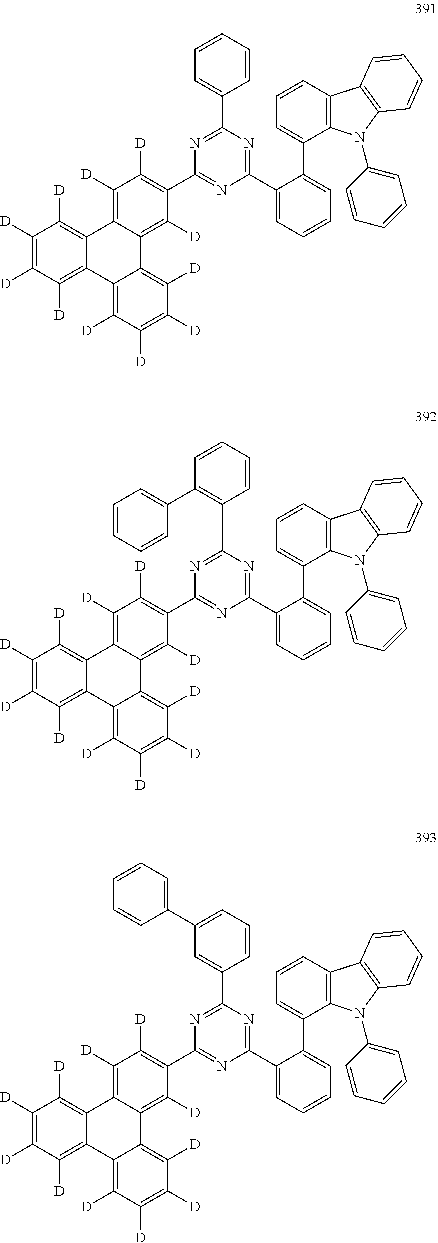 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c6ccccc6n(-c6ccccc6)c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c6ccccc6n(-c6ccccc6)c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c6ccccc6n(-c6ccccc6)c45)n3)c([2H])c([2H])c21]

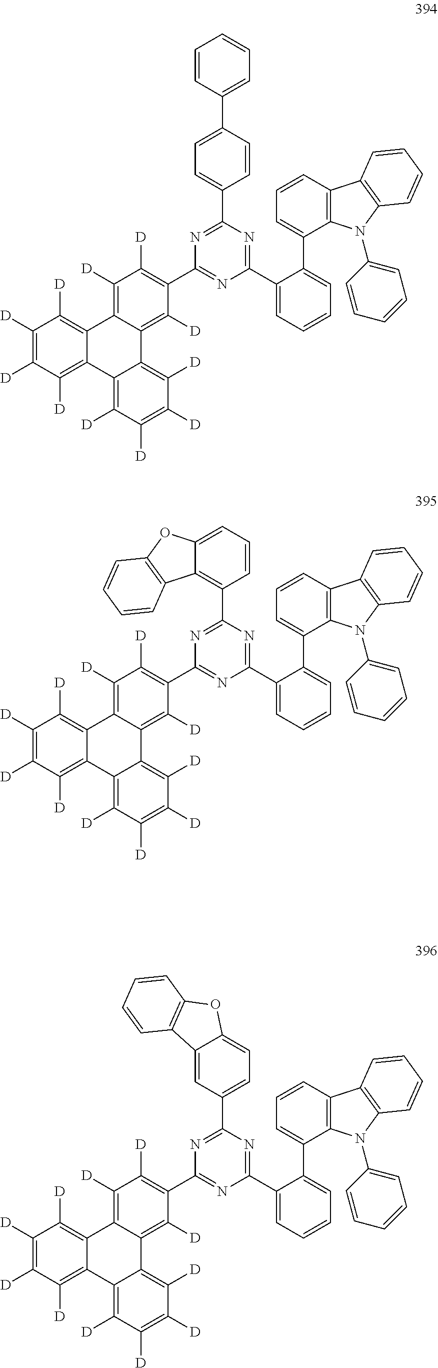

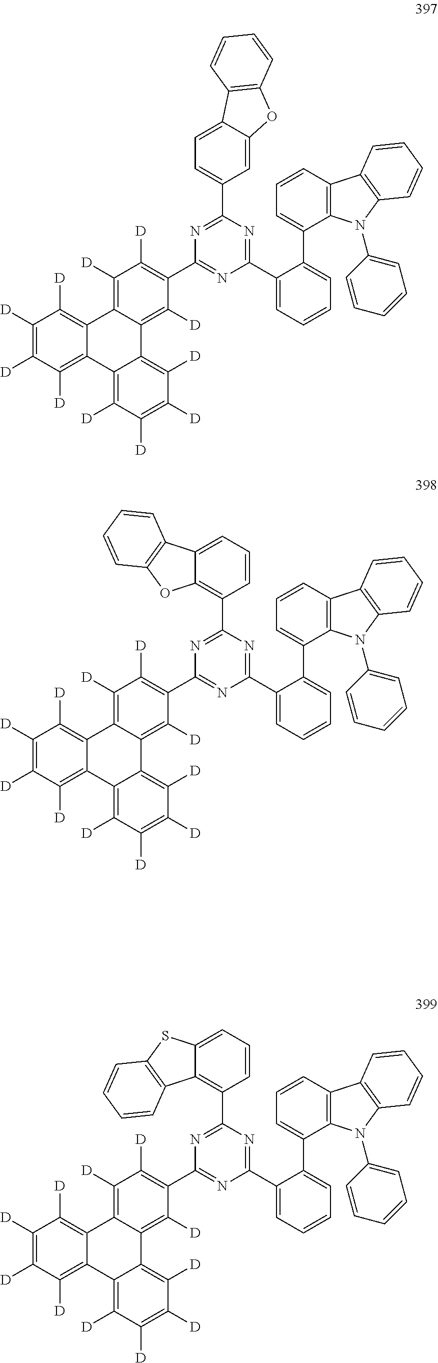

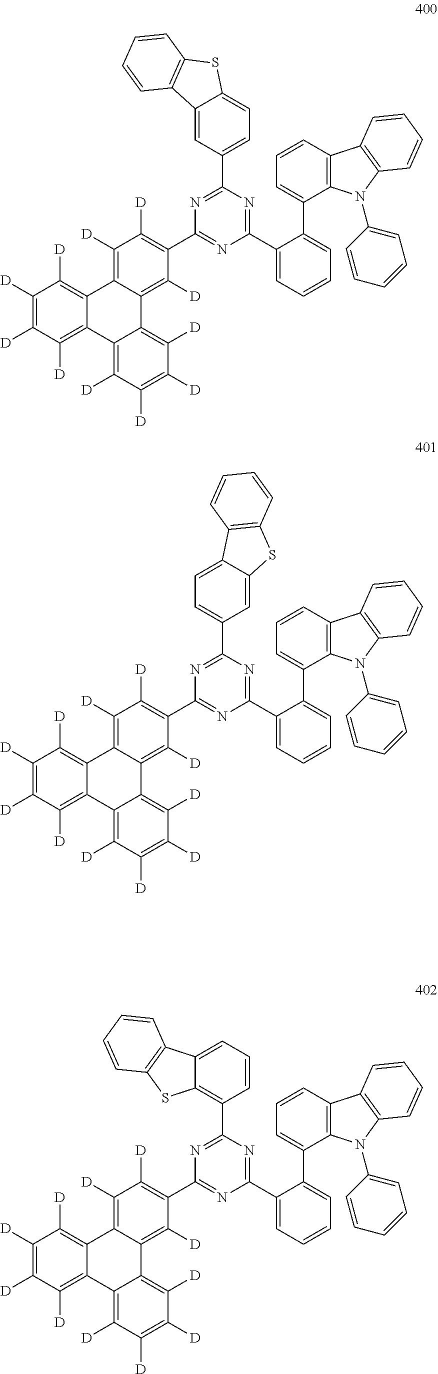

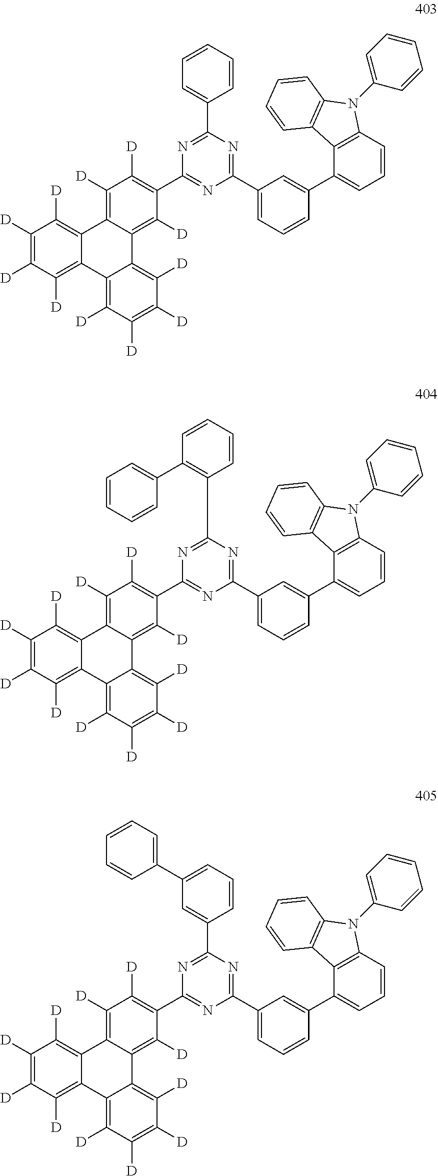 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c5c5ccccc5n6-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c5c5ccccc5n6-c5ccccc5)c4)n3)c([2H])c([2H])c21]

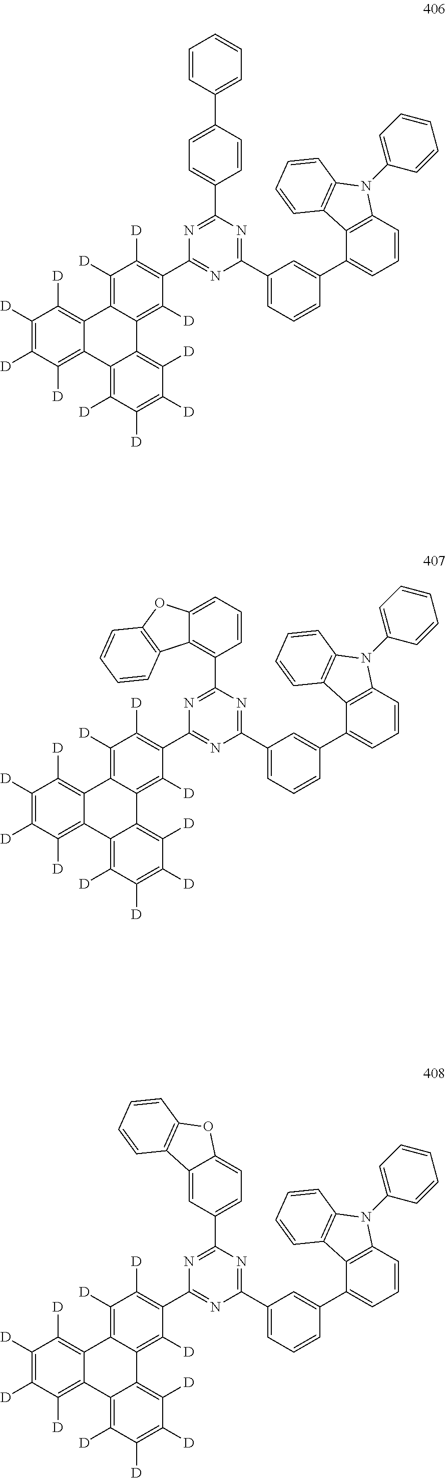

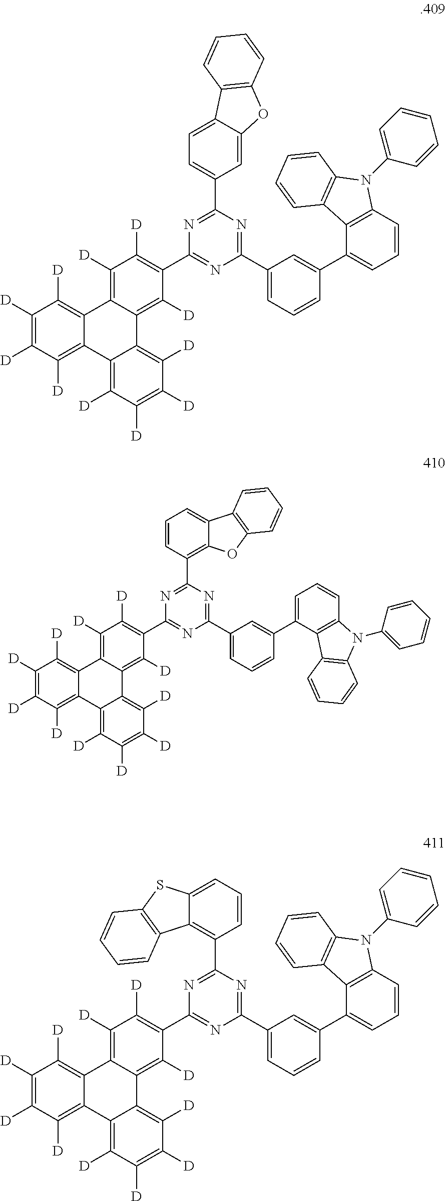

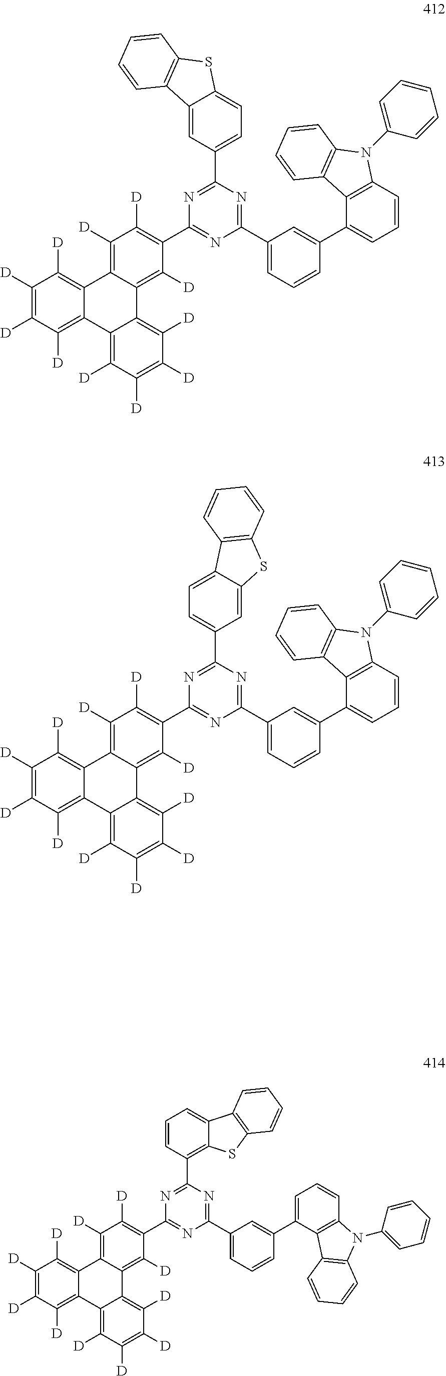

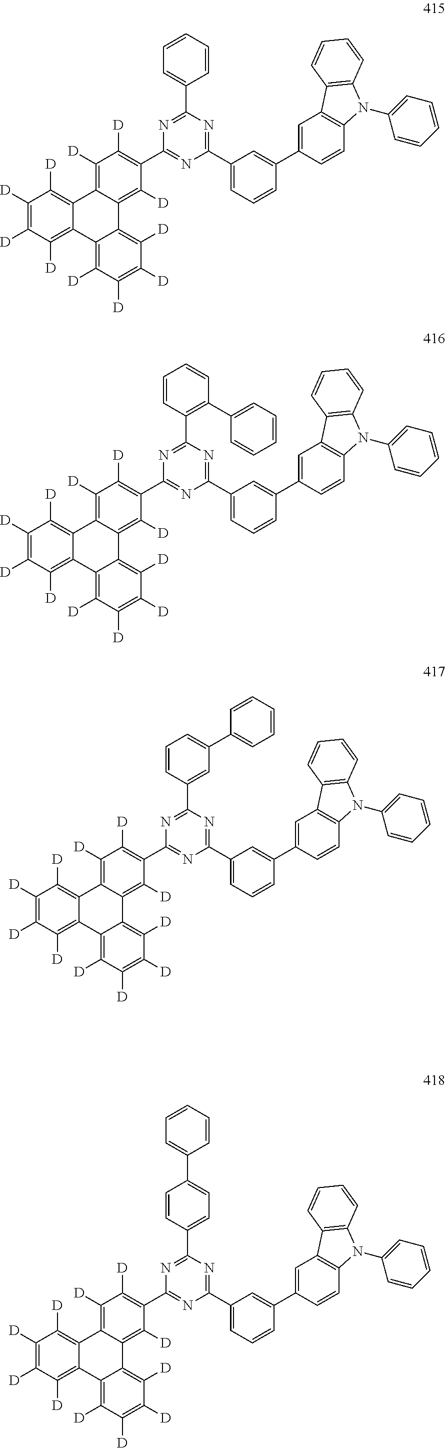

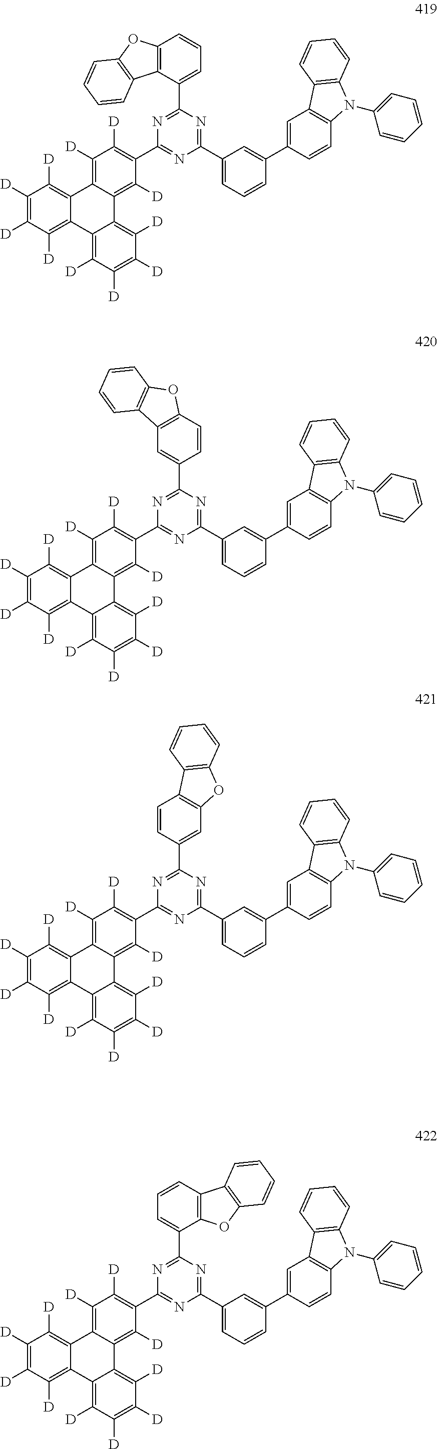

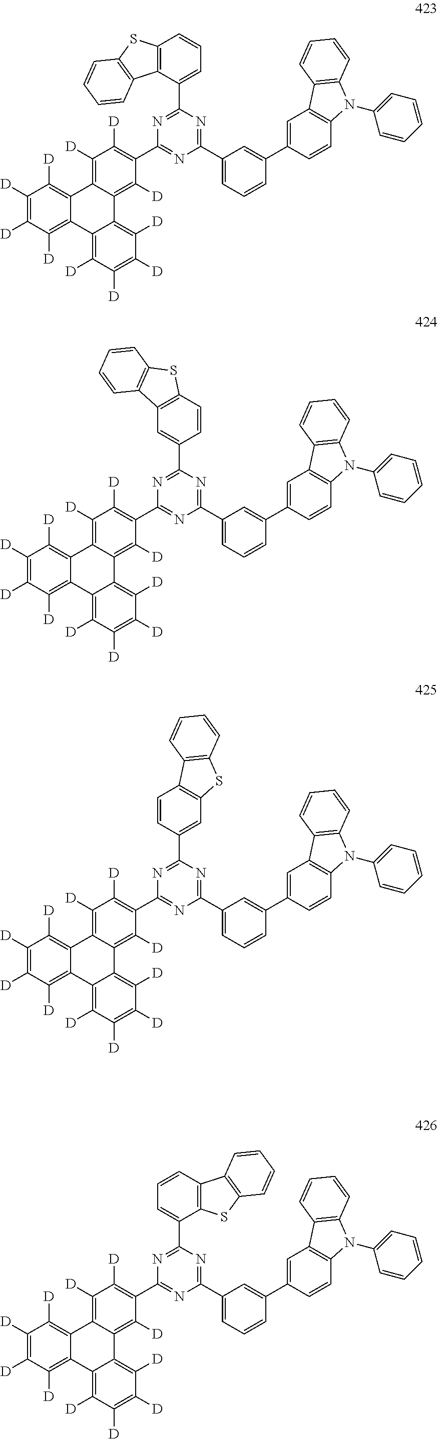

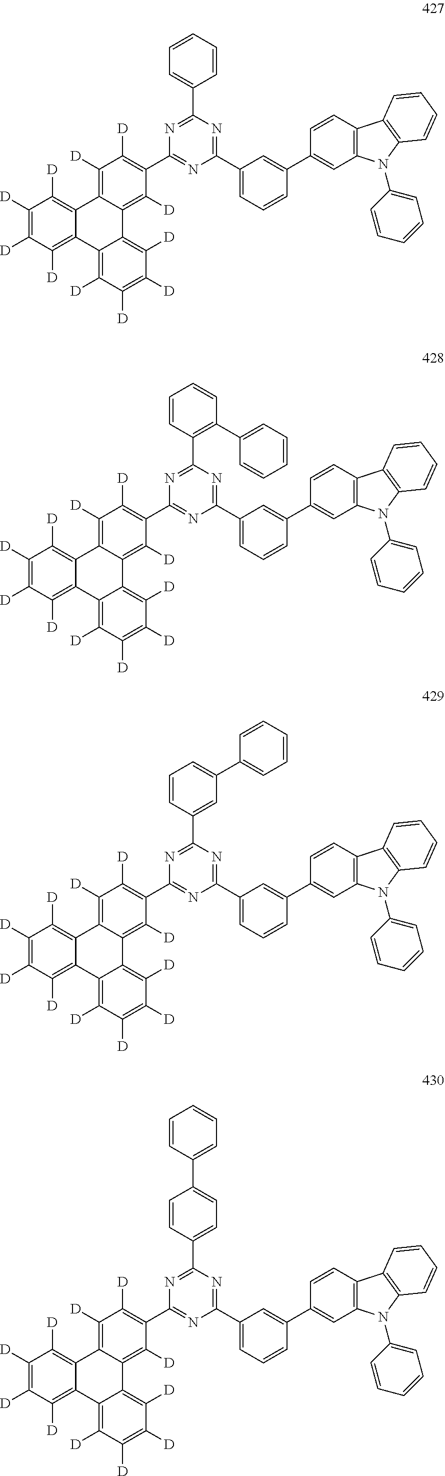

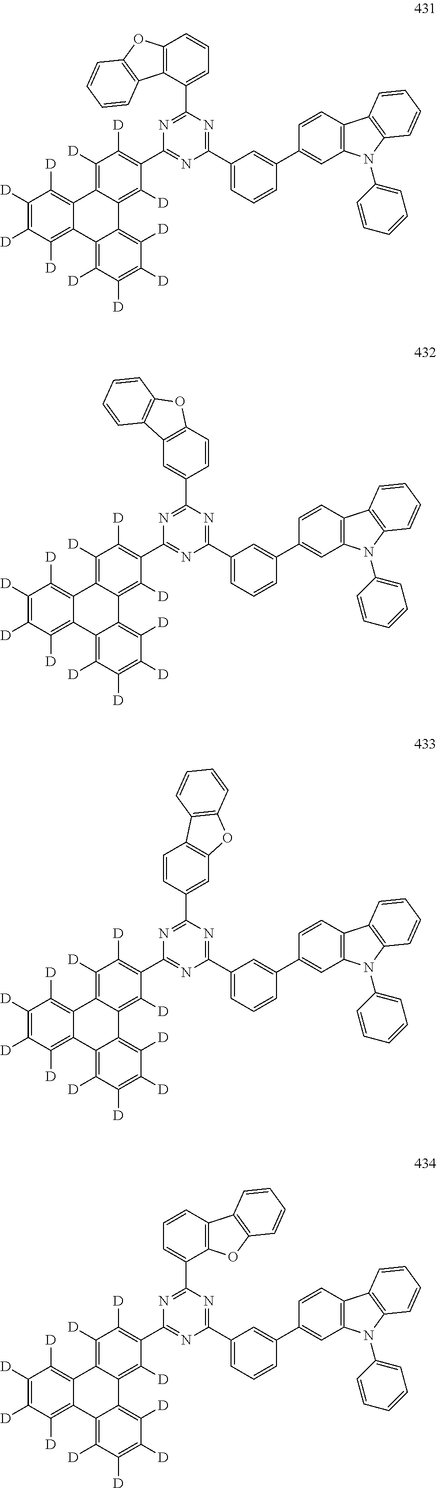

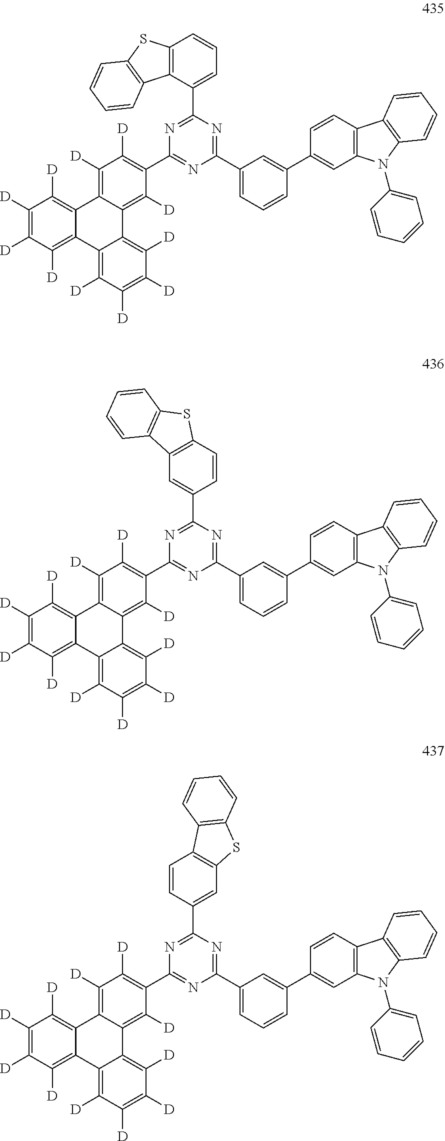

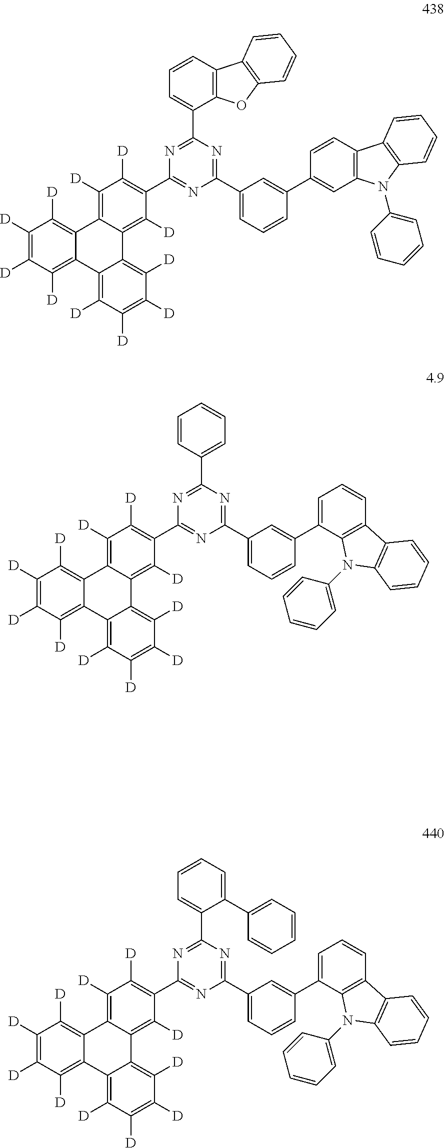 [SMILES: [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c7ccccc7n(-c7ccccc7)c56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4ccccc4-c4ccccc4)n3)c([2H])c([2H])c21]

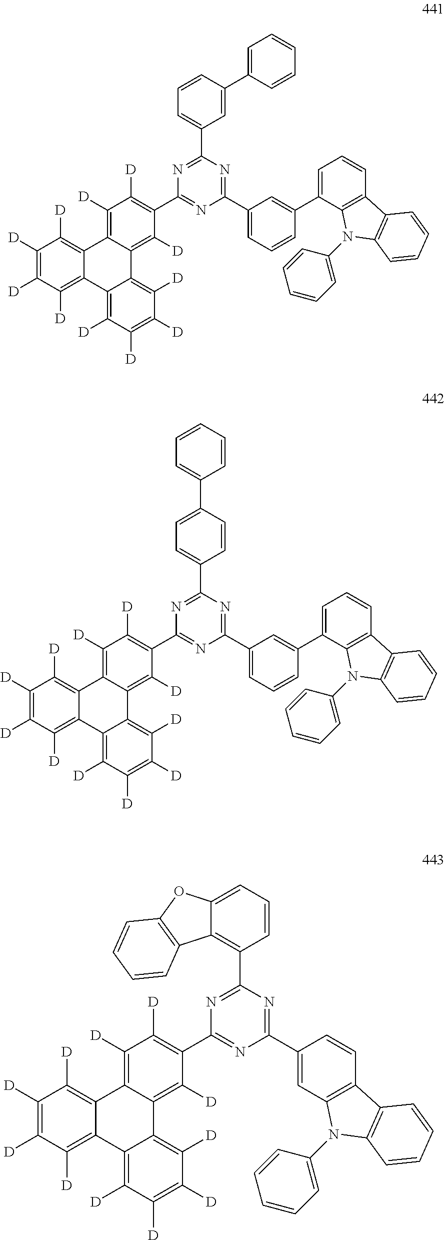 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c7ccccc7n(-c7ccccc7)c56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5cccc6c7ccccc7n(-c7ccccc7)c56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c6ccccc6n(-c6ccccc6)c5c4)nc(-c4cccc5oc6ccccc6c45)n3)c([2H])c([2H])c21]

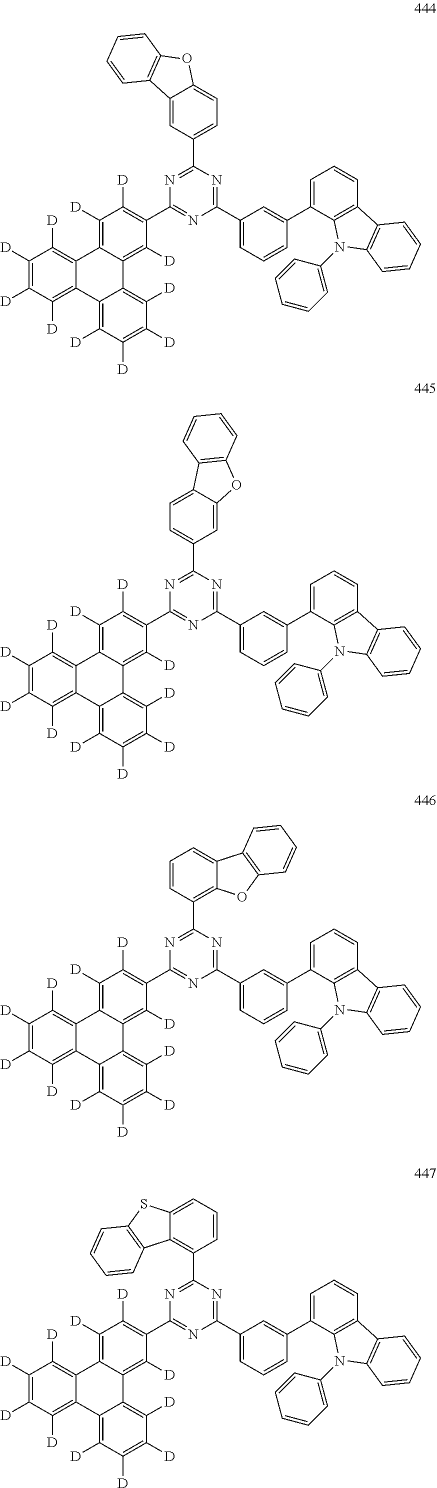

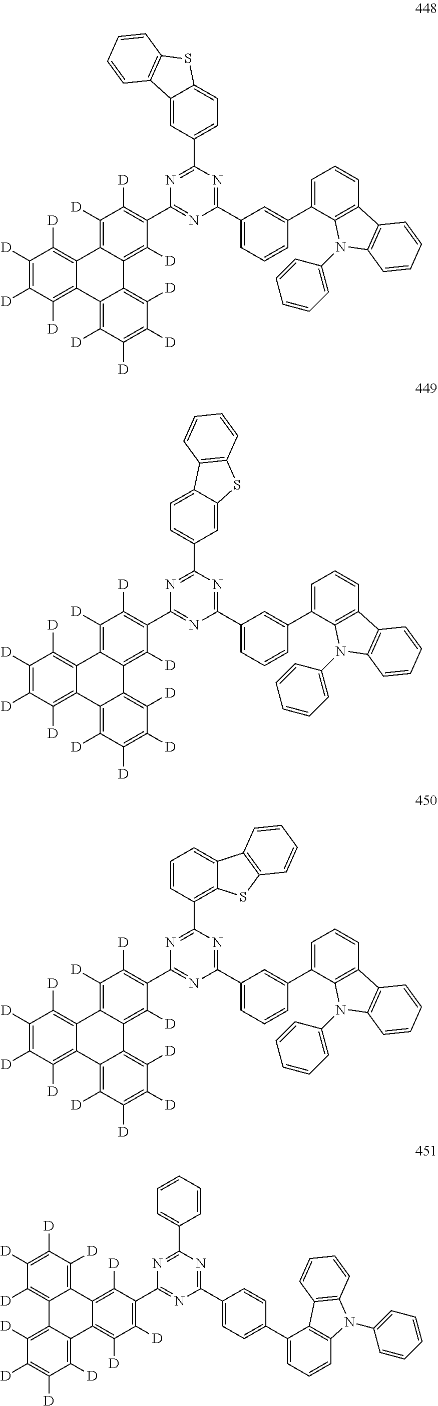

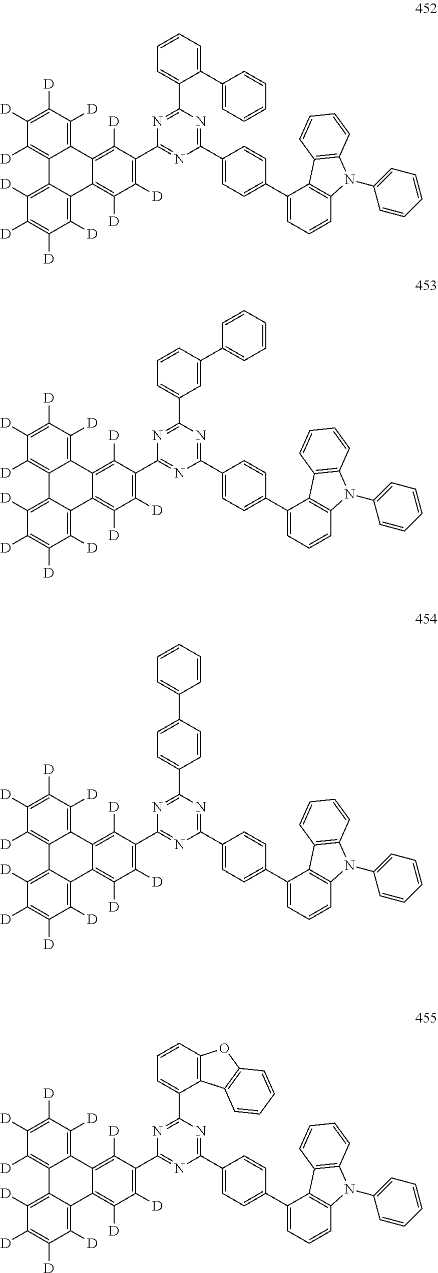

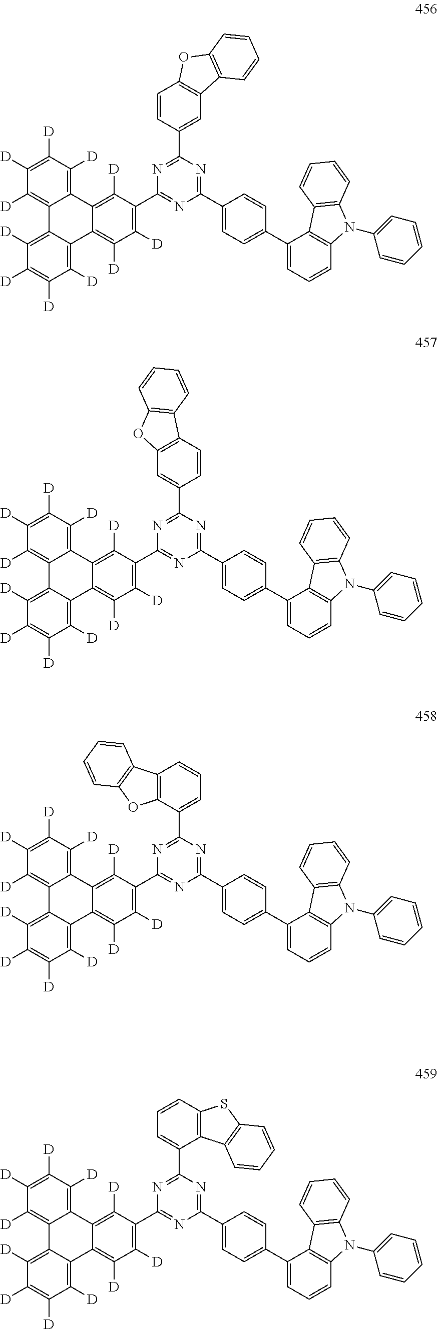

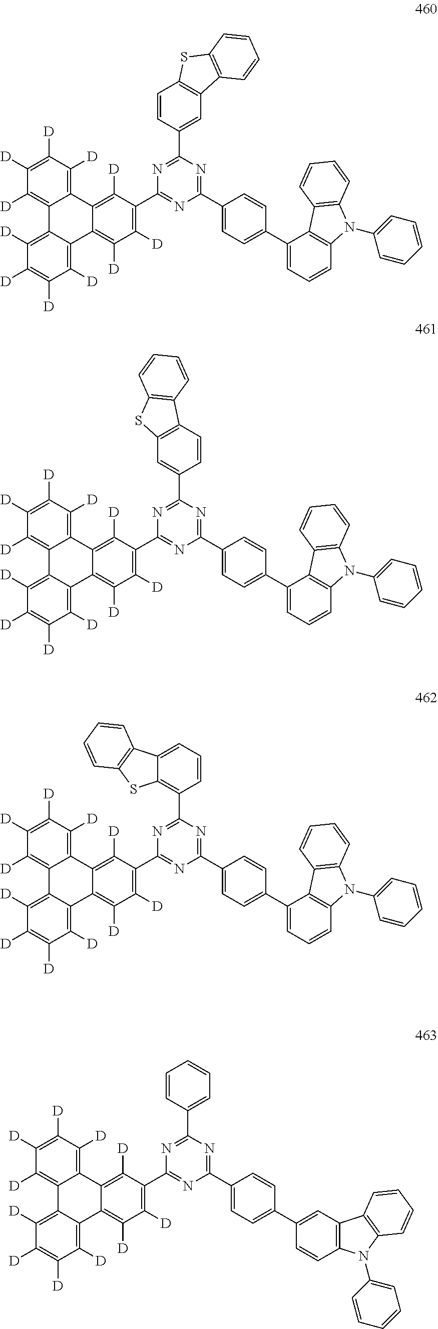

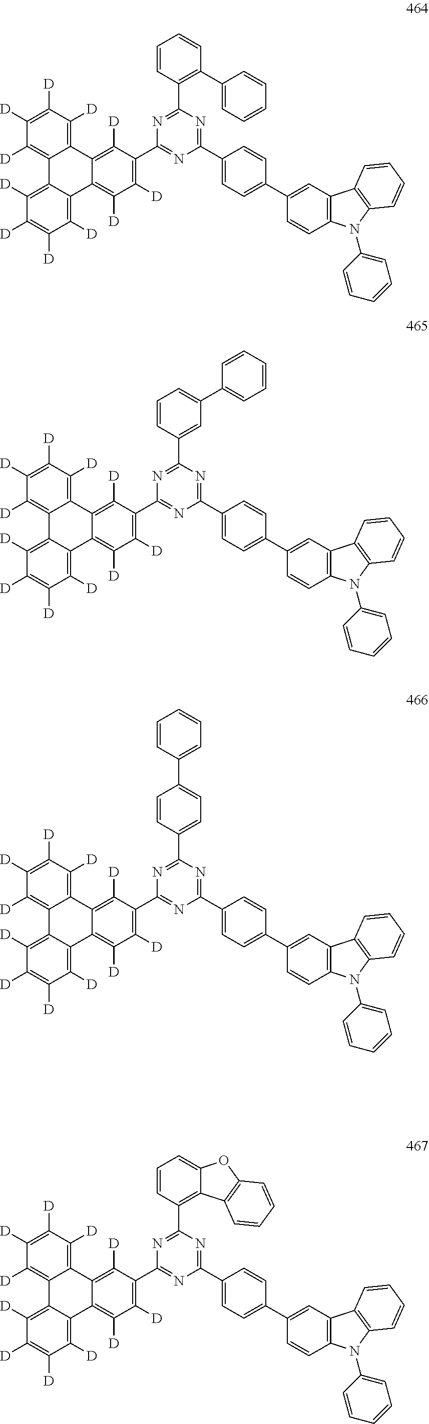

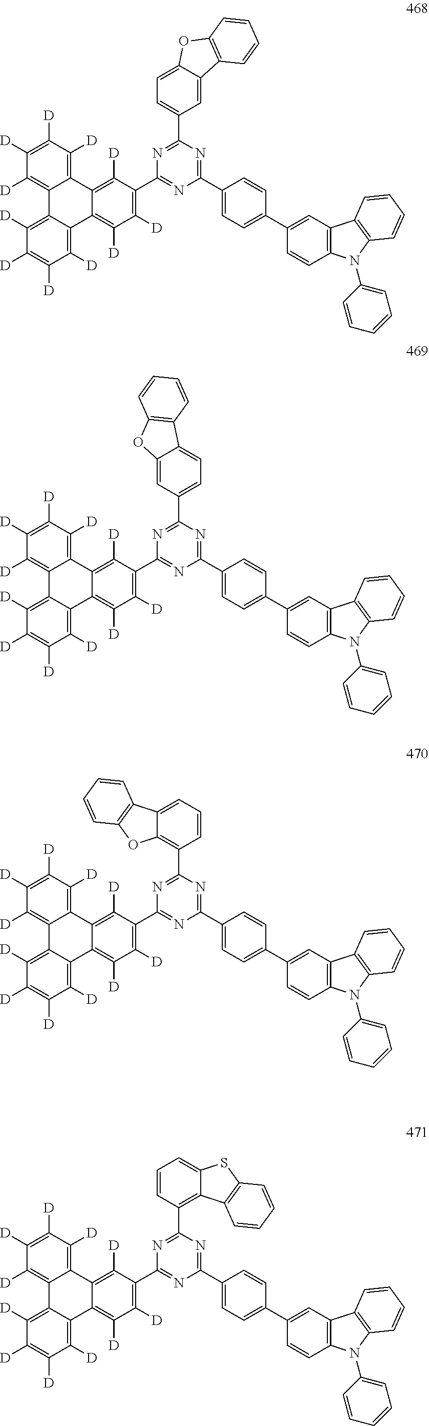

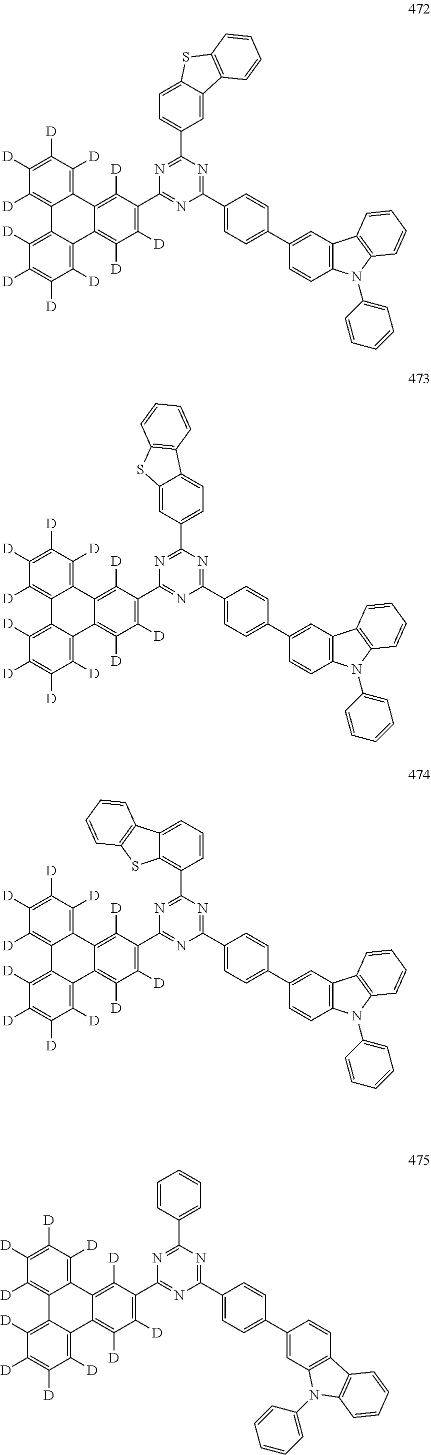

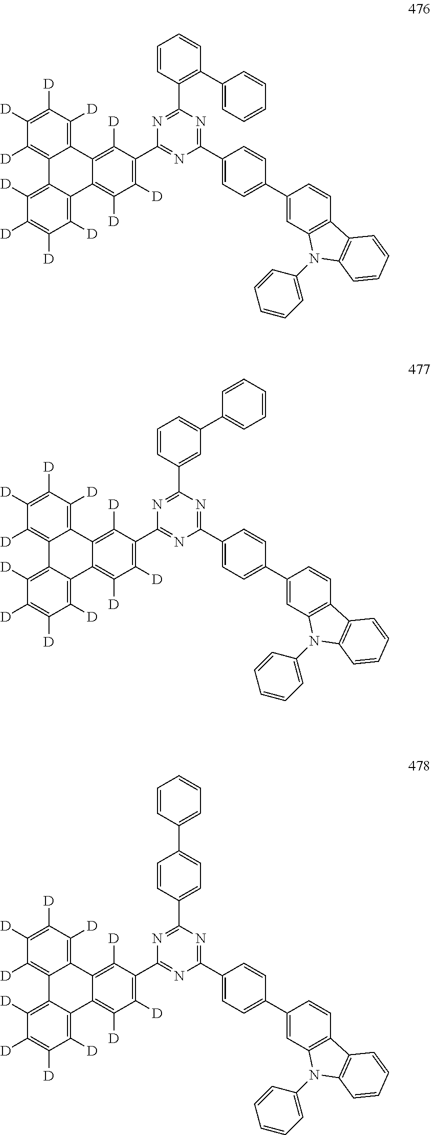

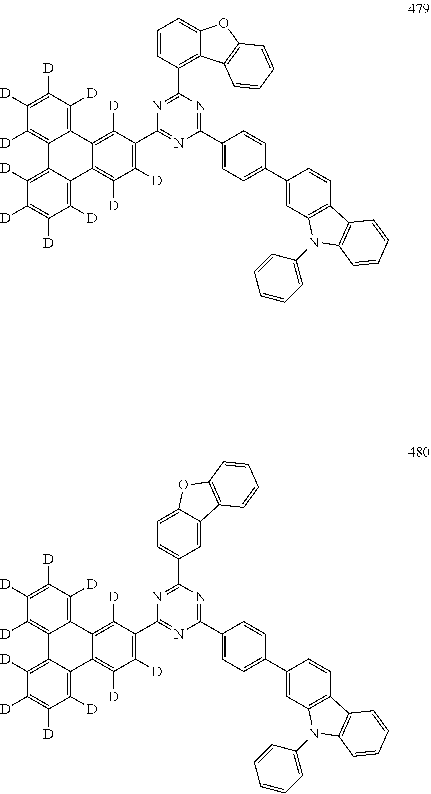 [SMILES: [2H]c1c([2H])c([2H])c2c(c1[2H])c1c([2H])c([2H])c([2H])c([2H])c1c1c([2H])c(-c3nc(-c4ccc(-c5ccc6c7ccccc7n(-c7ccccc7)c6c5)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c7ccccc7n(-c7ccccc7)c6c5)cc4)nc(-c4ccc5oc6ccccc6c5c4)n3)c([2H])c([2H])c21]

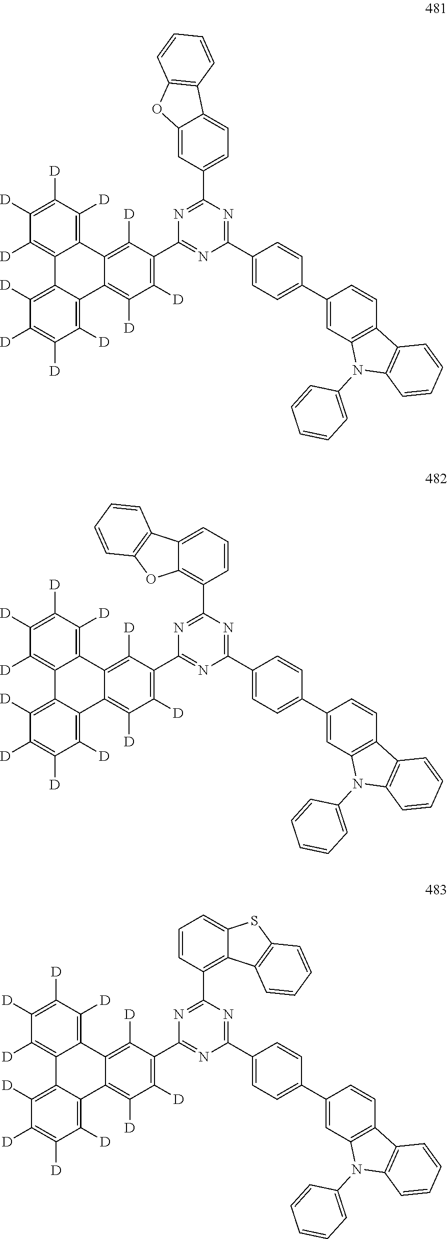

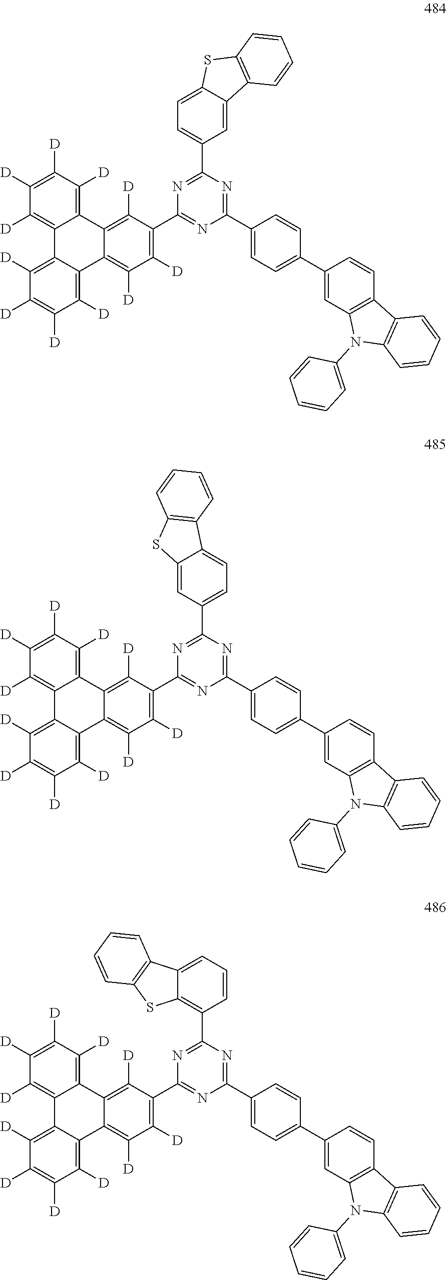

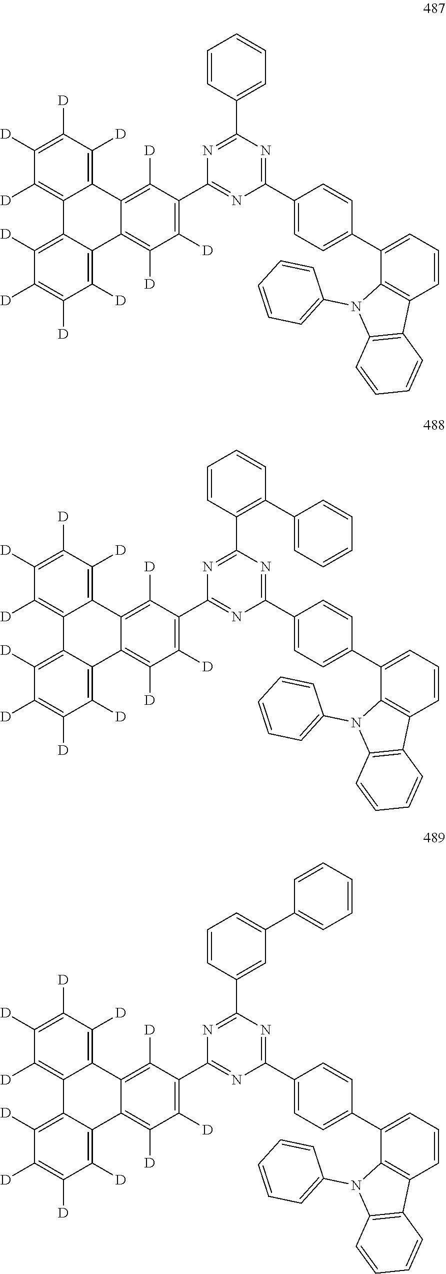 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c7ccccc7n(-c7ccccc7)c56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c7ccccc7n(-c7ccccc7)c56)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c7ccccc7n(-c7ccccc7)c56)cc4)nc(-c4cccc(-c5ccccc5)c4)n3)c([2H])c([2H])c21]

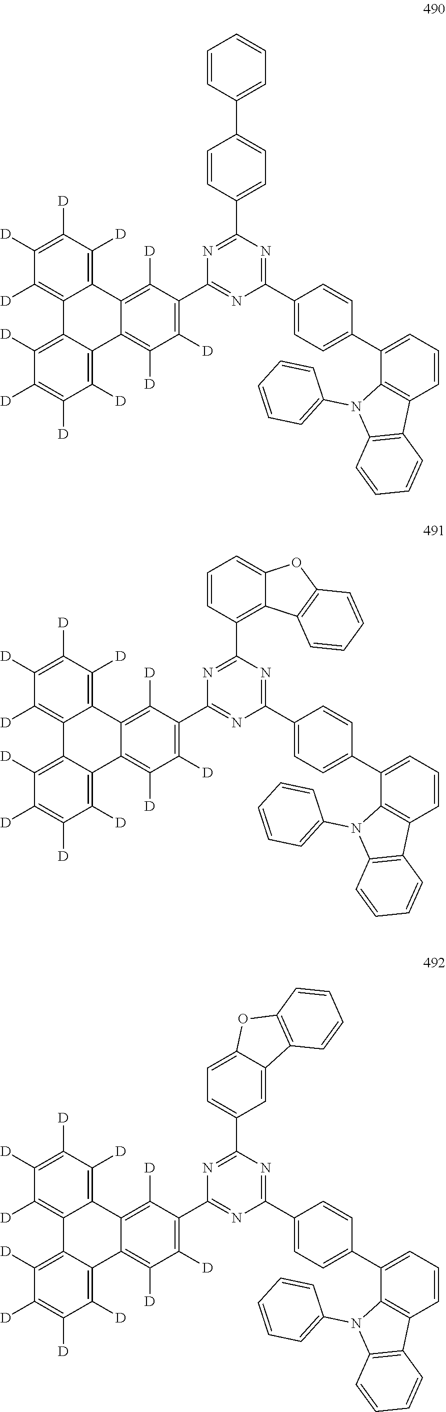

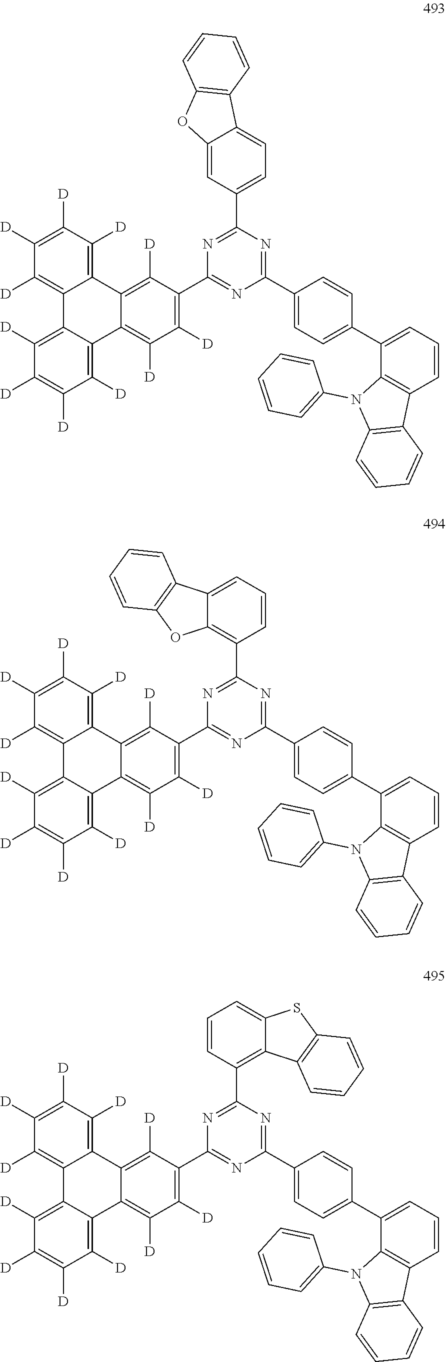

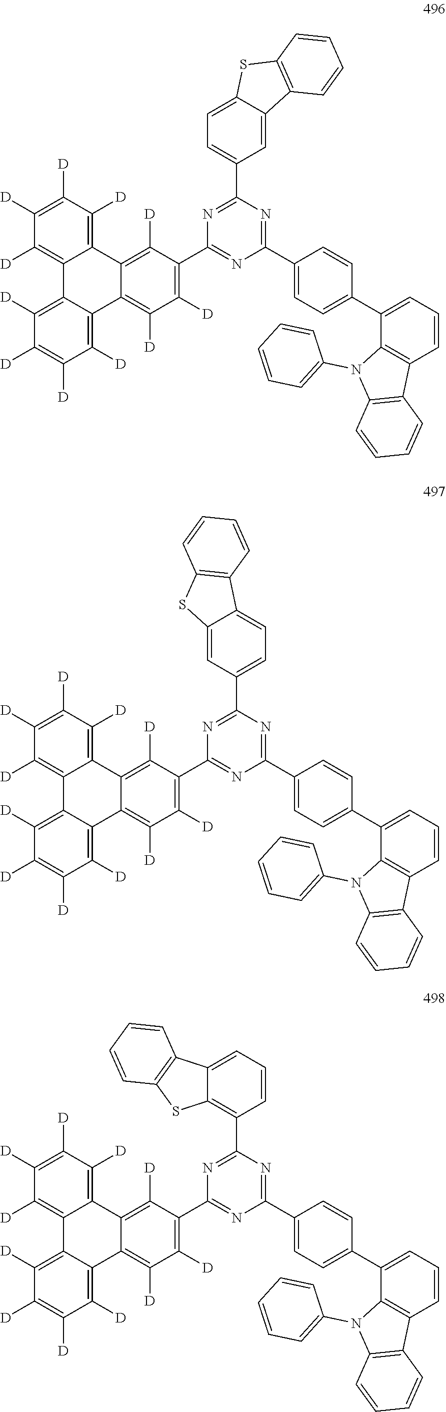

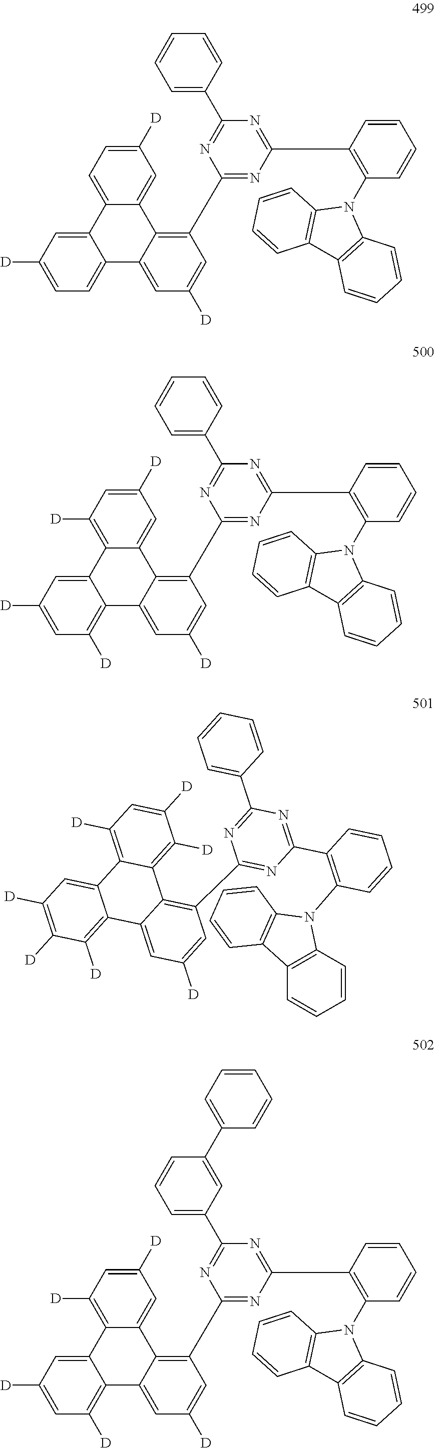 [SMILES: [2H]c1ccc2c(c1)c1ccc([2H])cc1c1c(-c3nc(-c4ccccc4)nc(-c4ccccc4-n4c5ccccc5c5ccccc54)n3)cc([2H])cc21 ; [2H]c1cc([2H])c2c(c1)c1c([2H])cc([2H])cc1c1c(-c3nc(-c4ccccc4)nc(-c4ccccc4-n4c5ccccc5c5ccccc54)n3)cc([2H])cc21 ; [2H]c1cc(-c2nc(-c3ccccc3)nc(-c3ccccc3-n3c4ccccc4c4ccccc43)n2)c2c(c1)c1c([2H])c([2H])c([2H])cc1c1c([2H])cc([2H])c([2H])c12 ; [2H]c1cc([2H])c2c(c1)c1c([2H])cc([2H])cc1c1c(-c3nc(-c4cccc(-c5ccccc5)c4)nc(-c4ccccc4-n4c5ccccc5c5ccccc54)n3)cc([2H])cc21]

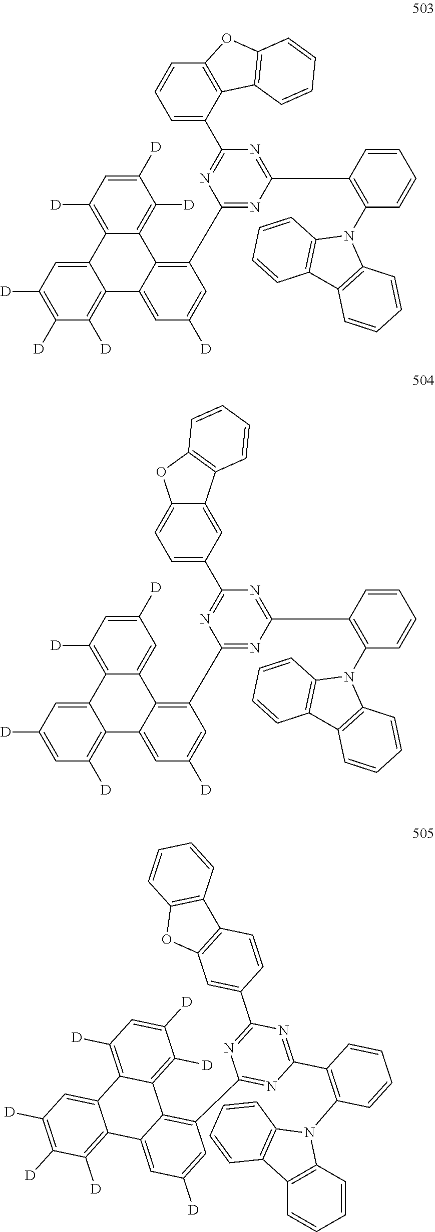 [SMILES: [2H]c1cc(-c2nc(-c3ccccc3-n3c4ccccc4c4ccccc43)nc(-c3cccc4oc5ccccc5c34)n2)c2c(c1)c1c([2H])c([2H])c([2H])cc1c1c([2H])cc([2H])c([2H])c12 ; [2H]c1cc([2H])c2c(c1)c1c([2H])cc([2H])cc1c1c(-c3nc(-c4ccc5oc6ccccc6c5c4)nc(-c4ccccc4-n4c5ccccc5c5ccccc54)n3)cc([2H])cc21 ; [2H]c1cc(-c2nc(-c3ccc4c(c3)oc3ccccc34)nc(-c3ccccc3-n3c4ccccc4c4ccccc43)n2)c2c(c1)c1c([2H])c([2H])c([2H])cc1c1c([2H])cc([2H])c([2H])c12]

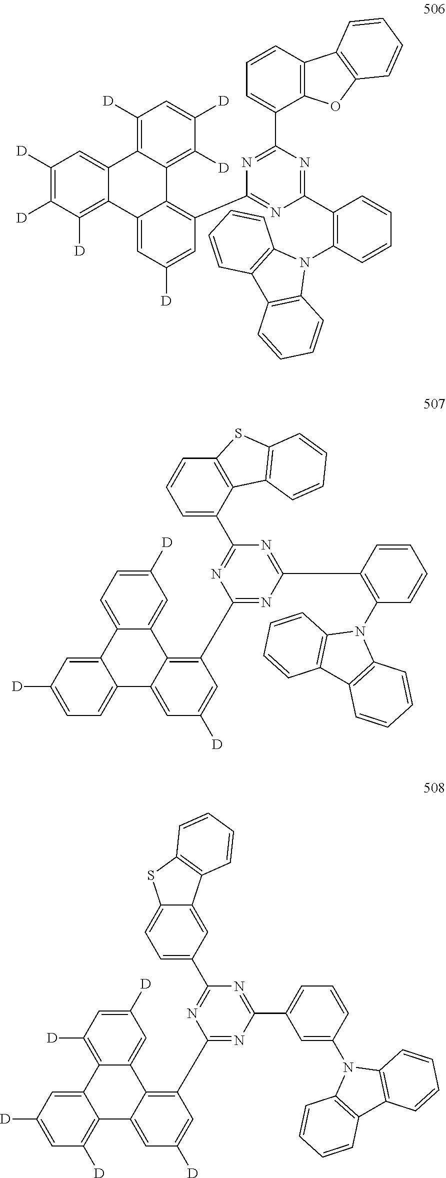 [SMILES: [2H]c1cc(-c2nc(-c3ccccc3-n3c4ccccc4c4ccccc43)nc(-c3cccc4c3oc3ccccc34)n2)c2c(c1)c1c([2H])c([2H])c([2H])cc1c1c([2H])cc([2H])c([2H])c12 ; [2H]c1ccc2c(c1)c1ccc([2H])cc1c1c(-c3nc(-c4ccccc4-n4c5ccccc5c5ccccc54)nc(-c4cccc5sc6ccccc6c45)n3)cc([2H])cc21 ; [2H]c1cc([2H])c2c(c1)c1c([2H])cc([2H])cc1c1c(-c3nc(-c4cccc(-n5c6ccccc6c6ccccc65)c4)nc(-c4ccc5sc6ccccc6c5c4)n3)cc([2H])cc21]

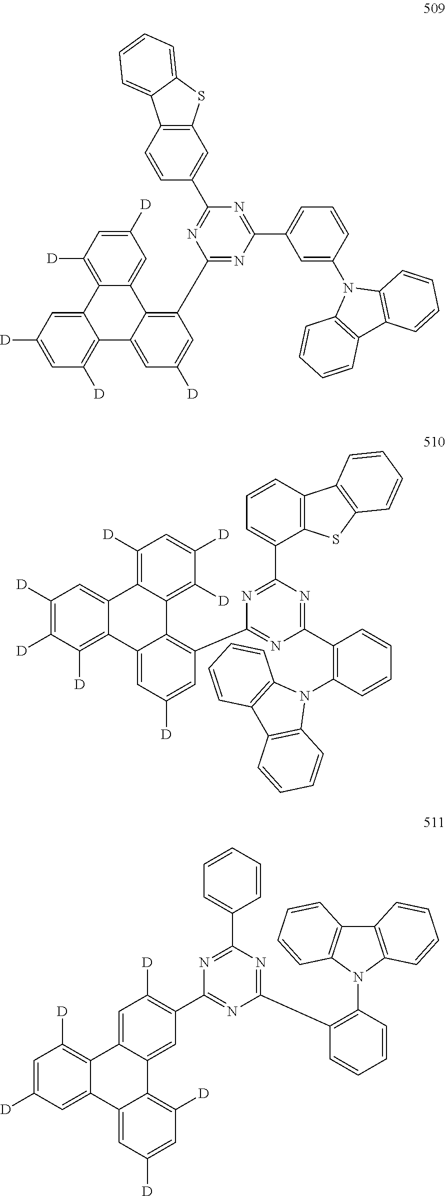 [SMILES: [2H]c1cc([2H])c2c(c1)c1c([2H])cc([2H])cc1c1c(-c3nc(-c4cccc(-n5c6ccccc6c6ccccc65)c4)nc(-c4ccc5c(c4)sc4ccccc45)n3)cc([2H])cc21 ; [2H]c1cc(-c2nc(-c3ccccc3-n3c4ccccc4c4ccccc43)nc(-c3cccc4c3sc3ccccc34)n2)c2c(c1)c1c([2H])c([2H])c([2H])cc1c1c([2H])cc([2H])c([2H])c12 ; [2H]c1cc([2H])c2c(c1)c1cc([2H])cc([2H])c1c1cc(-c3nc(-c4ccccc4)nc(-c4ccccc4-n4c5ccccc5c5ccccc54)n3)c([2H])cc12]

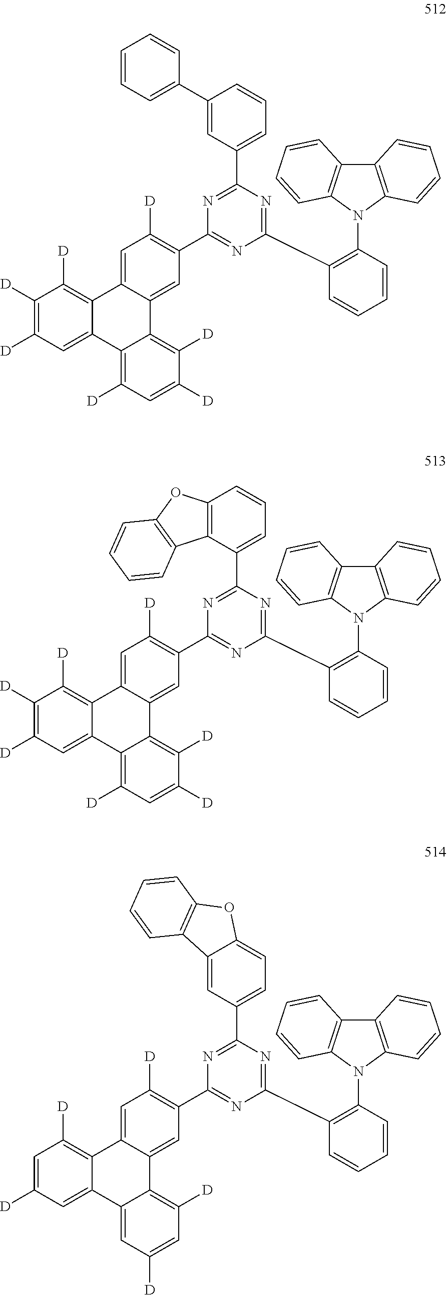 [SMILES: [2H]c1cc2c(cc1-c1nc(-c3cccc(-c4ccccc4)c3)nc(-c3ccccc3-n3c4ccccc4c4ccccc43)n1)c1c([2H])c([2H])cc([2H])c1c1cc([2H])c([2H])c([2H])c21 ; [2H]c1cc2c(cc1-c1nc(-c3ccccc3-n3c4ccccc4c4ccccc43)nc(-c3cccc4oc5ccccc5c34)n1)c1c([2H])c([2H])cc([2H])c1c1cc([2H])c([2H])c([2H])c21 ; [2H]c1cc([2H])c2c(c1)c1cc([2H])cc([2H])c1c1cc(-c3nc(-c4ccc5oc6ccccc6c5c4)nc(-c4ccccc4-n4c5ccccc5c5ccccc54)n3)c([2H])cc12]

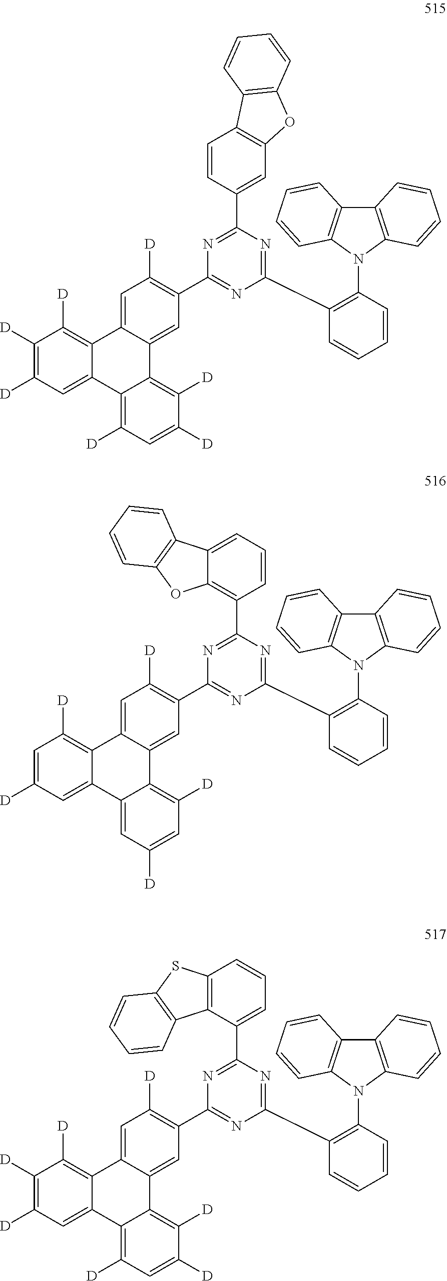 [SMILES: [2H]c1cc2c(cc1-c1nc(-c3ccc4c(c3)oc3ccccc34)nc(-c3ccccc3-n3c4ccccc4c4ccccc43)n1)c1c([2H])c([2H])cc([2H])c1c1cc([2H])c([2H])c([2H])c21 ; [2H]c1cc([2H])c2c(c1)c1cc([2H])cc([2H])c1c1cc(-c3nc(-c4ccccc4-n4c5ccccc5c5ccccc54)nc(-c4cccc5c4oc4ccccc45)n3)c([2H])cc12 ; [2H]c1cc2c(cc1-c1nc(-c3ccccc3-n3c4ccccc4c4ccccc43)nc(-c3cccc4sc5ccccc5c34)n1)c1c([2H])c([2H])cc([2H])c1c1cc([2H])c([2H])c([2H])c21]

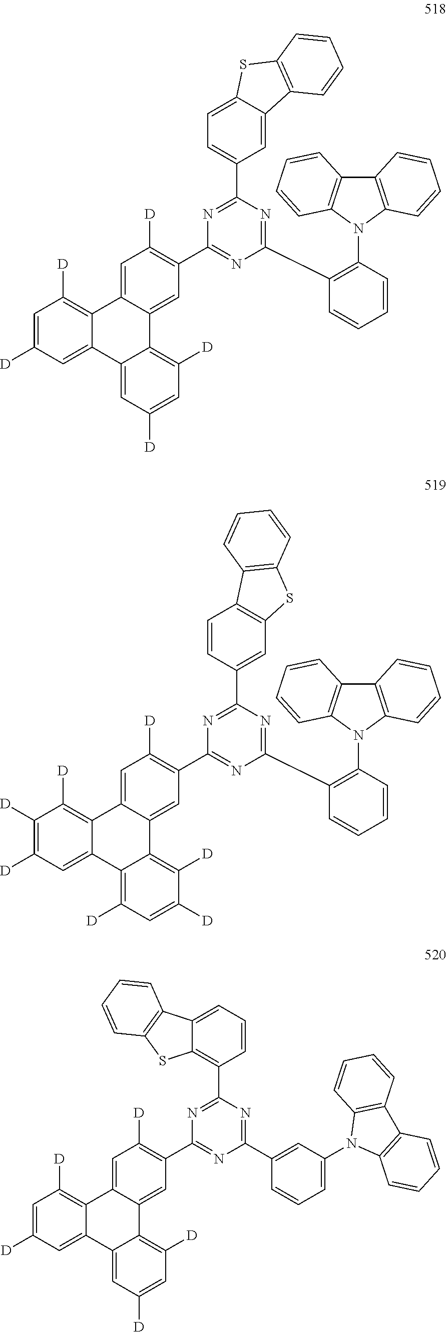 [SMILES: [2H]c1cc([2H])c2c(c1)c1cc([2H])cc([2H])c1c1cc(-c3nc(-c4ccc5sc6ccccc6c5c4)nc(-c4ccccc4-n4c5ccccc5c5ccccc54)n3)c([2H])cc12 ; [2H]c1cc2c(cc1-c1nc(-c3ccc4c(c3)sc3ccccc34)nc(-c3ccccc3-n3c4ccccc4c4ccccc43)n1)c1c([2H])c([2H])cc([2H])c1c1cc([2H])c([2H])c([2H])c21 ; [2H]c1cc([2H])c2c(c1)c1cc([2H])cc([2H])c1c1cc(-c3nc(-c4cccc(-n5c6ccccc6c6ccccc65)c4)nc(-c4cccc5c4sc4ccccc45)n3)c([2H])cc12]

As long as the unique characteristics of the heterocyclic compound represented by Chemical Formula 1 are maintained, various substituents may be introduced in addition to the structures exemplified above to synthesize a heterocyclic compound to which the characteristics of the introduced substituents are added. For example, it is possible to synthesize a material which satisfies the conditions required for each organic material layer by introducing into the core structure a substituent usually used for a hole injection layer material, a hole transport layer material, a hole transport auxiliary layer material, a light emitting layer material, an electron transport layer material, an electron transport auxiliary layer material, and an electron blocking layer material used during the manufacture of an organic light emitting device.

In addition, it is possible to finely adjust an energy band-gap by introducing various substituents into the heterocyclic compound structure represented by Chemical Formula 1, and meanwhile, it is possible to improve characteristics at the interface between organic materials and diversify the use of the material.

### <Organic Light Emitting Device>

The organic light emitting device according to an exemplary embodiment of the present specification is an organic light emitting device including: a first electrode; a second electrode provided to face the first electrode; and an organic material layer having one or more layers provided between the first electrode and the second electrode, and one or more layers of the organic material layers may include the above-described heterocyclic compound (represented by Chemical Formula 1).

According to an exemplary embodiment of the present specification, one or more layers of the organic material layer include a light emitting layer, and the light emitting layer may include the heterocyclic compound (represented by Chemical Formula 1).

According to an exemplary embodiment of the present specification, the organic material layer further includes a hole transport layer, and the hole transport layer may include the heterocyclic compound (represented by Chemical Formula 1).

According to an exemplary embodiment of the present specification, the organic material layer further includes an electron blocking layer, and the electron blocking layer may include the heterocyclic compound (represented by Chemical Formula 1).

In another exemplary embodiment of the present specification, the light emitting layer may include the heterocyclic compound represented by Chemical Formula 1 as a host.

According to an exemplary embodiment of the present specification, the light emitting layer may include the heterocyclic compound represented by Chemical Formula 1 as a red host.

According to an exemplary embodiment of the present specification, the light emitting layer may include the heterocyclic compound represented by Chemical Formula 1 as a green host.

According to an exemplary embodiment of the present specification, the light emitting layer may include the heterocyclic compound represented by Chemical Formula 1 as a blue host.

According to an exemplary embodiment of the present specification, one or more layers of the organic material layer may further include a heterocyclic compound represented by the following Chemical Formula 2 or 3.

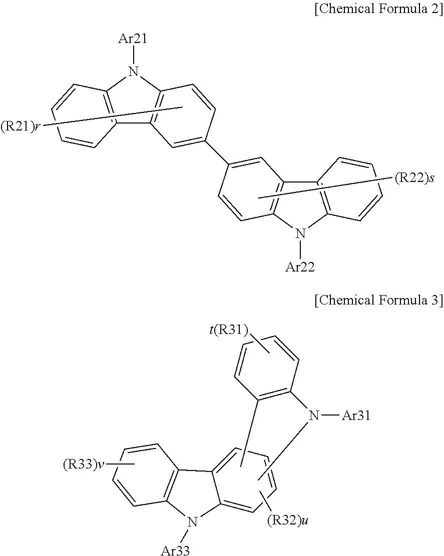

In Chemical Formulae 2 and 3,

R21, R22, and R31 to R33 are the same as or different from each other, and are each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR′R″R′″; —P(═O)R′R″; and an amine group which is unsubstituted or substituted with a substituted or unsubstituted C1 to C60 alkyl group, a substituted or unsubstituted C6 to C60 aryl group or a substituted or unsubstituted C2 to C60 heteroaryl group, or two or more adjacent groups are bonded to each other to form a substituted or unsubstituted C6 to C60 aliphatic or aromatic hydrocarbon ring or a substituted or unsubstituted C2 to C60 aliphatic or aromatic hetero ring,
R′, R″, and R′″ are the same as or different from each other, and are each independently hydrogen; deuterium; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r and s are each an integer from 0 to 7, and when each of r and s is 2 or higher, substituents in the parenthesis are the same as or different from each other,
t and v are each an integer from 0 to 4, and when each of t and v is 2 or higher, substituents in the parenthesis are the same as or different from each other,
u is an integer from 0 to 2, and when u is 2, substituents in the parenthesis are the same as or different from each other, and
Ar21, Ar22, Ar31, and Ar33 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

According to the exemplary embodiments, when the heterocyclic compound represented by Chemical Formula 1 and the heterocyclic compound represented by Chemical Formula 2 or 3 are simultaneously included, an exciplex phenomenon occurs, so that the driving voltage of an organic light emitting device may be further lowered, and the light emitting efficiency and service life may be further improved.

According to an exemplary embodiment of the present specification, R21, R22, and R31 to R33 are the same as or different from each other, and are each independently selected from the group consisting of hydrogen; deuterium; a halogen group; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —SiR′R″R′″; —P(═O)R′R″; and an amine group which is unsubstituted or substituted with a substituted or unsubstituted C1 to C40 alkyl group, a substituted or unsubstituted C6 to C40 aryl group or a substituted or unsubstituted C2 to C40 heteroaryl group, or two or more adjacent groups may be bonded to each other to form a substituted or unsubstituted C6 to C40 aliphatic or aromatic hydrocarbon ring or a substituted or unsubstituted C2 to C40 aliphatic or aromatic hetero ring.

According to an exemplary embodiment of the present specification, R21, R22, and 31 to R33 are the same as or different from each other, and are each independently selected from the group consisting of hydrogen; deuterium; a halogen group; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —SiR′R″R′″; —P(═O)R′R″; and an amine group which is unsubstituted or substituted with a substituted or unsubstituted C1 to C30 alkyl group, a substituted or unsubstituted C6 to C30 aryl group or a substituted or unsubstituted C2 to C30 heteroaryl group, or two or more adjacent groups are bonded to each other to form a substituted or unsubstituted C6 to C30 aliphatic or aromatic hydrocarbon ring or a substituted or unsubstituted C2 to C30 aliphatic or aromatic hetero ring.

According to an exemplary embodiment of the present specification, R21, R22, and R31 to R33 are the same as or different from each other, and may be each independently hydrogen; or deuterium.

According to an exemplary embodiment of the present specification, Ar21, Ar22, Ar31, and Ar33 are the same as or different from each other, and may be each independently hydrogen; deuterium; a substituted or unsubstituted C1 to C40 alkyl group; a substituted or unsubstituted C6 to C40 aryl group; or a substituted or unsubstituted C2 to C40 heteroaryl group.

According to an exemplary embodiment of the present specification, Ar21, Ar22, Ar31, and Ar33 are the same as or different from each other, and may be each independently hydrogen; deuterium; a substituted or unsubstituted C1 to C30 alkyl group; a substituted or unsubstituted C6 to C30 aryl group; or a substituted or unsubstituted C2 to C30 heteroaryl group.

According to an exemplary embodiment of the present specification, Ar21, Ar22, Ar31, and Ar33 are the same as or different from each other, and may be each independently hydrogen; deuterium; a C6 to C30 aryl group unsubstituted or substituted with one or more substituents selected from the group consisting of deuterium, a cyano group, an alkyl group, an aryl group, a heteroaryl group, and —SiR′R″R′″; or a C2 to C30 heteroaryl group unsubstituted or substituted with one or more substituents selected from the group consisting of deuterium, a cyano group, an alkyl group, an aryl group, a heteroaryl group, and —SiR′R″R′″. H ere, R′, R″, and R′″ are the same as or different from each other, and are each independently hydrogen; deuterium; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group.

According to an exemplary embodiment of the present specification, Ar21, Ar22, Ar31, and Ar33 are the same as or different from each other, and may be each independently hydrogen; deuterium; a C6 to C30 aryl group unsubstituted or substituted with one or more substituents selected from the group consisting of deuterium, a cyano group, a methyl group, an ethyl group, a phenyl group, a biphenyl group, a terphenyl group, a naphthyl group, a triphenylenyl group, a dibenzofuran group, a dibenzothiophene group, and —SiR′R″R′″; or a C2 to C30 heteroaryl group unsubstituted or substituted with one or more substituents selected from the group consisting of deuterium, a methyl group, an ethyl group, a phenyl group, a biphenyl group, a terphenyl group, a triphenylenyl group, a dibenzofuran group, and a dibenzothiophene group. Here, R′, R″, and R′″ are the same as or different from each other, and are each independently hydrogen; deuterium; a substituted or unsubstituted C6 to C60 aryl group; or a substituted or unsubstituted C2 to C60 heteroaryl group.

According to an exemplary embodiment of the present specification, Ar21, Ar22, Ar31, and Ar33 are the same as or different from each other, and may be each independently hydrogen; deuterium; a phenyl group unsubstituted or substituted with one or more substituents selected from the group consisting of deuterium, a cyano group, and a triphenylsilane group; a biphenyl group unsubstituted or substituted with deuterium; a terphenyl group unsubstituted or substituted with deuterium; a naphthyl group unsubstituted or substituted with deuterium; a triphenylenyl group unsubstituted or substituted with deuterium; a 9,9-dimethyl-9H-fluorenyl group unsubstituted or substituted with deuterium; a dibenzofuran group unsubstituted or substituted with one or more substituents selected from the group consisting deuterium and a phenyl group; or a dibenzothiophene group unsubstituted or substituted with one or more substituents selected from the group consisting of deuterium and a phenyl group.

According to an exemplary embodiment of the present specification, the heterocyclic compound represented by Chemical Formula 2 or 3 may be any one of the followings.

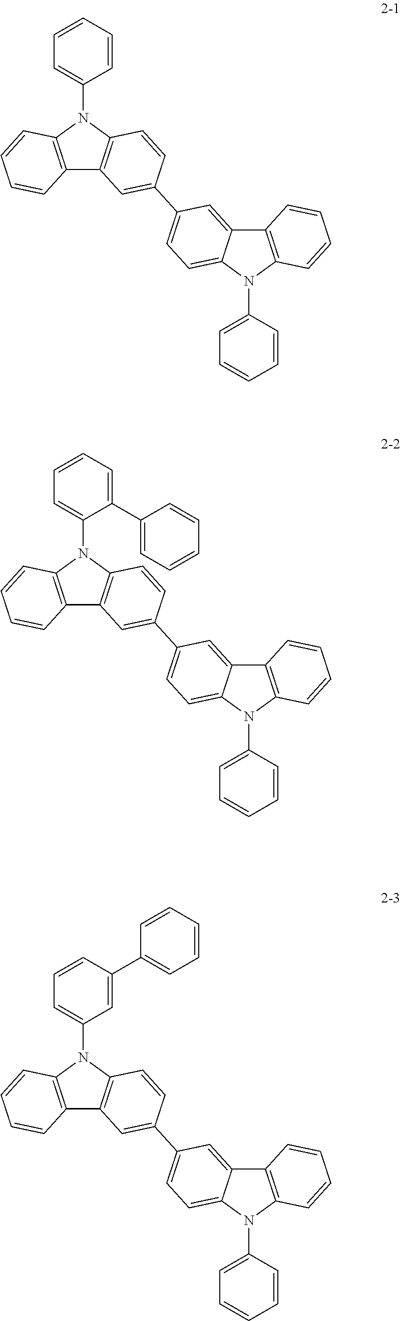 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1]

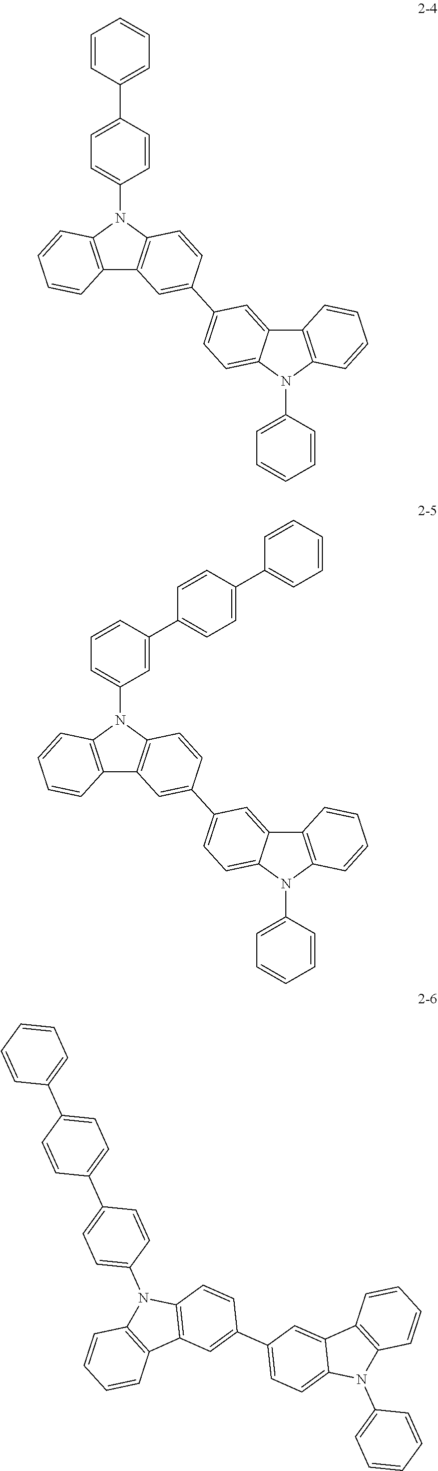

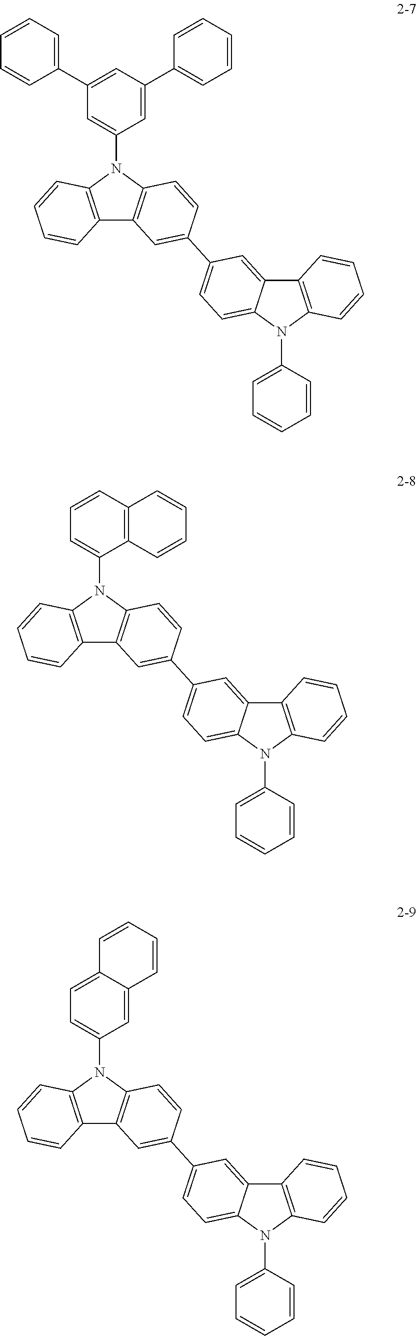 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1]

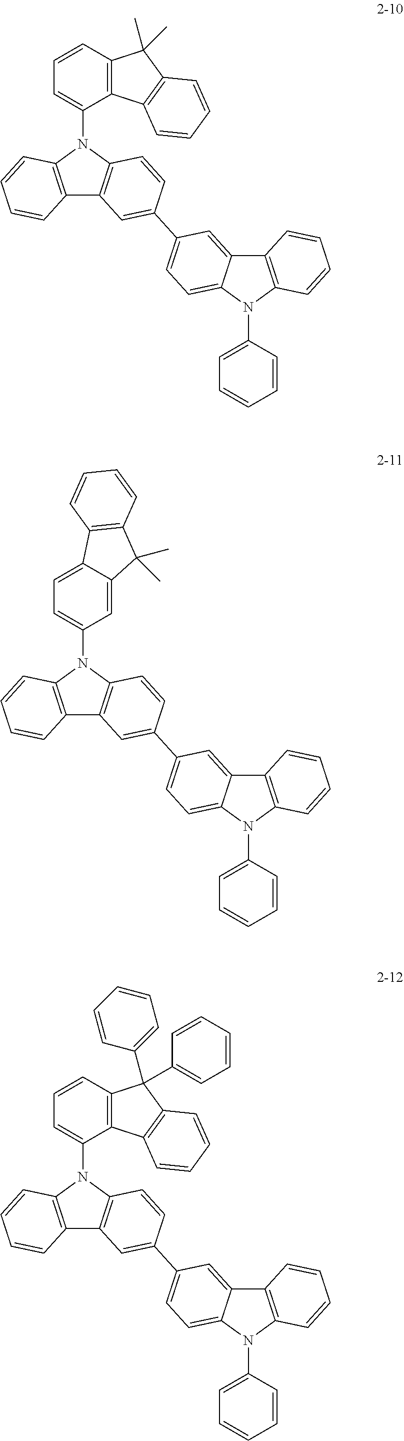 [SMILES: CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

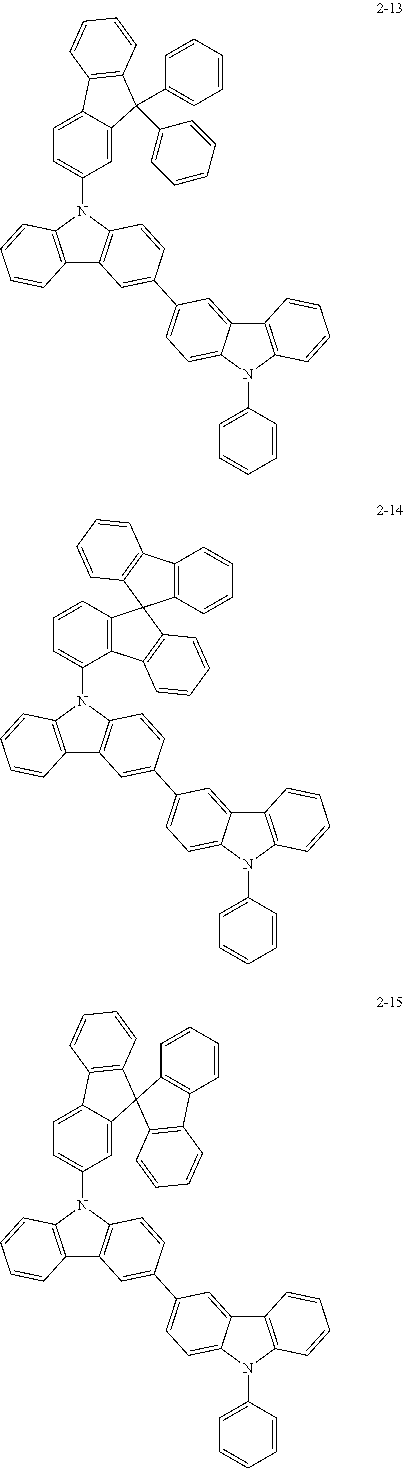

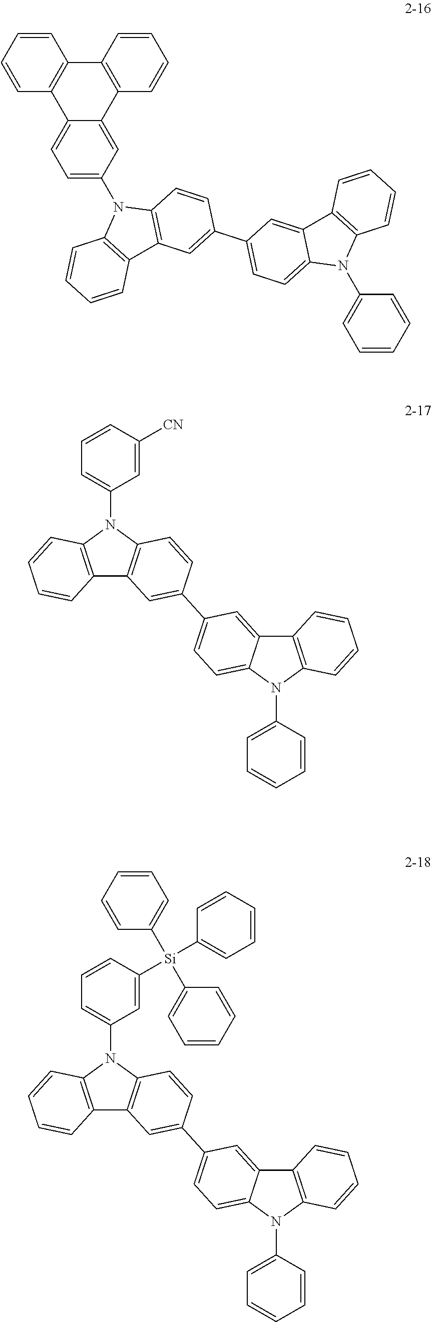 [SMILES: c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

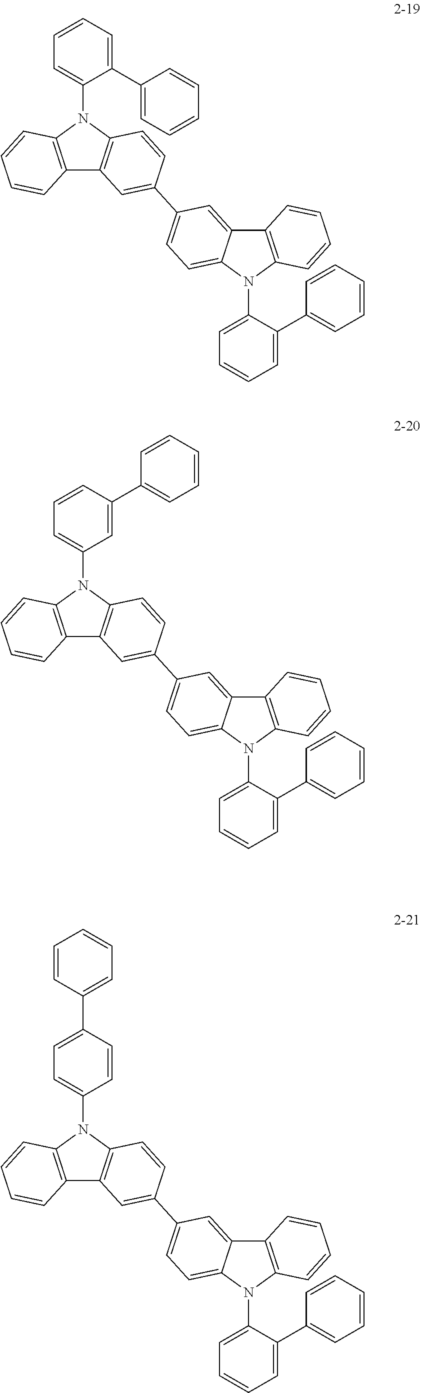 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

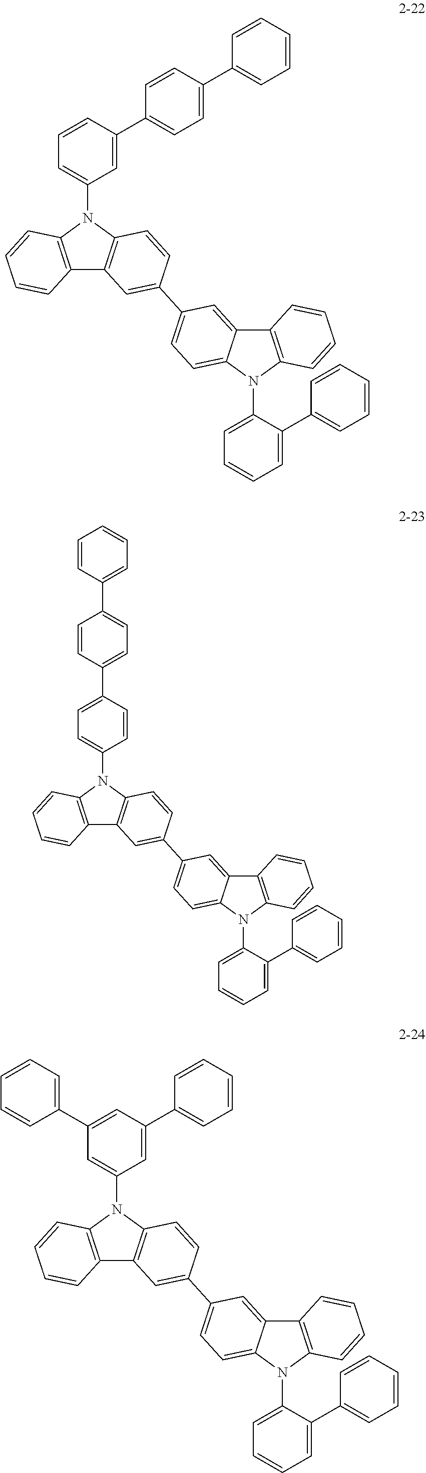 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

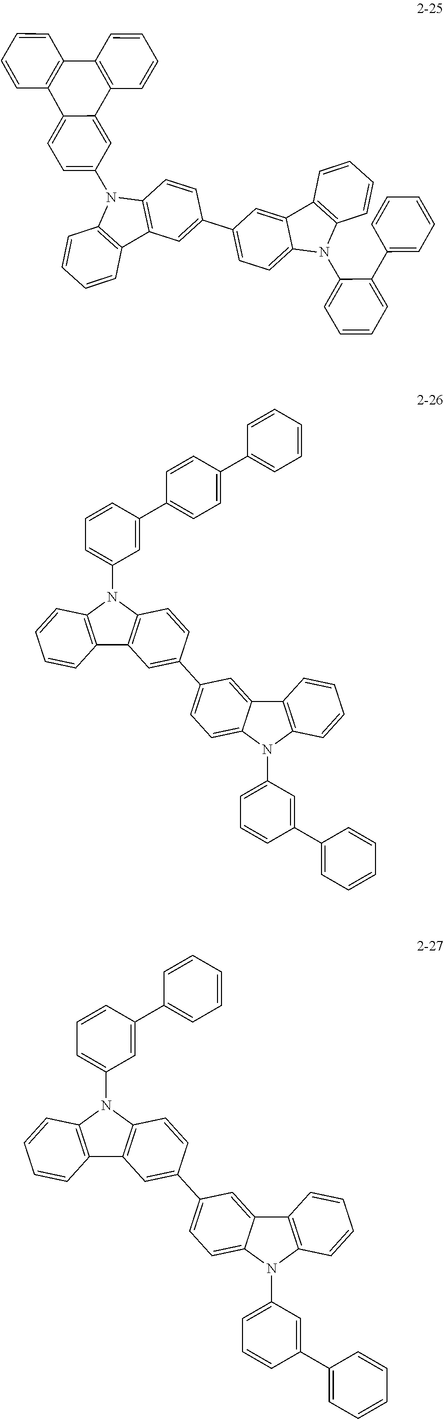 [SMILES: c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

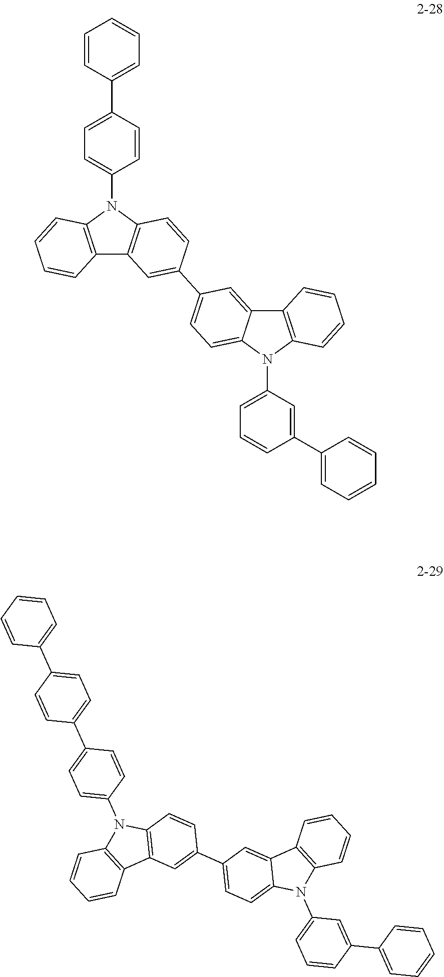 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

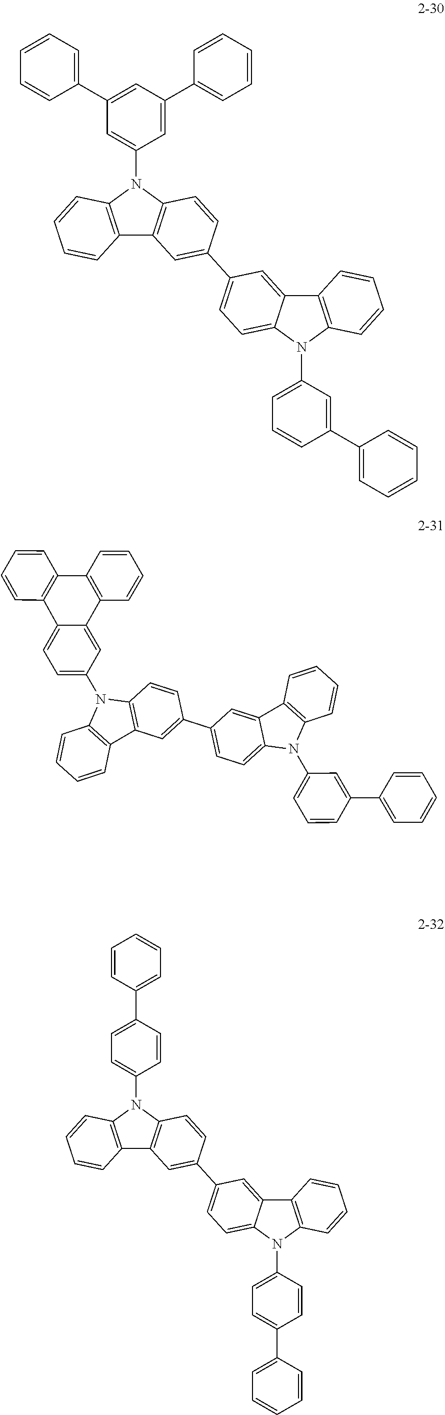 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

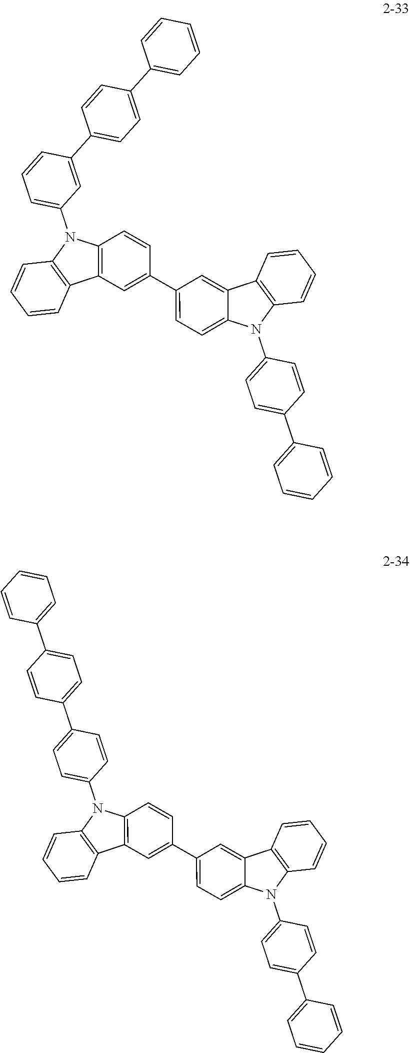 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

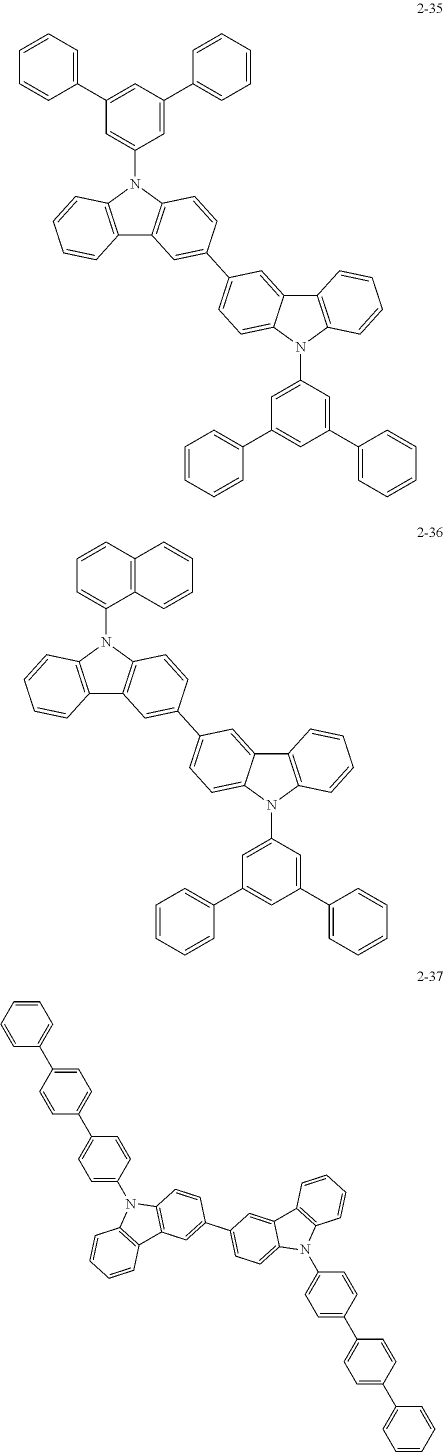 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1]

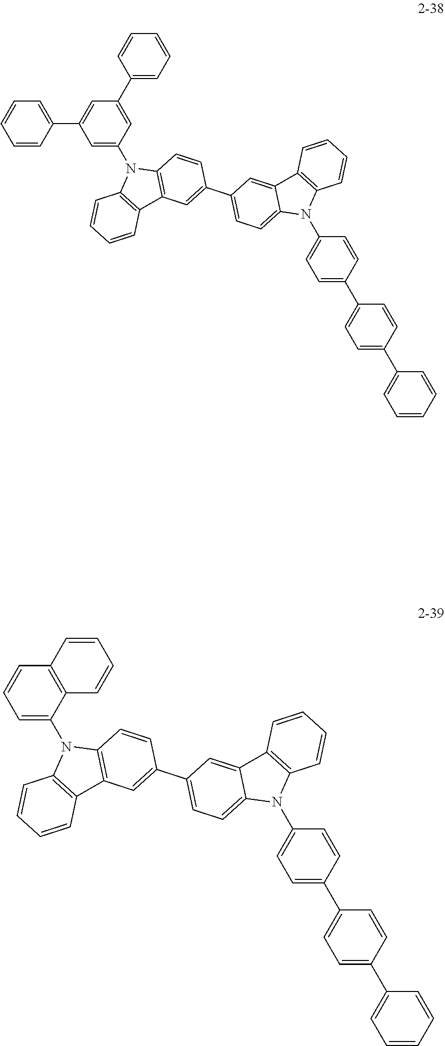 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1]

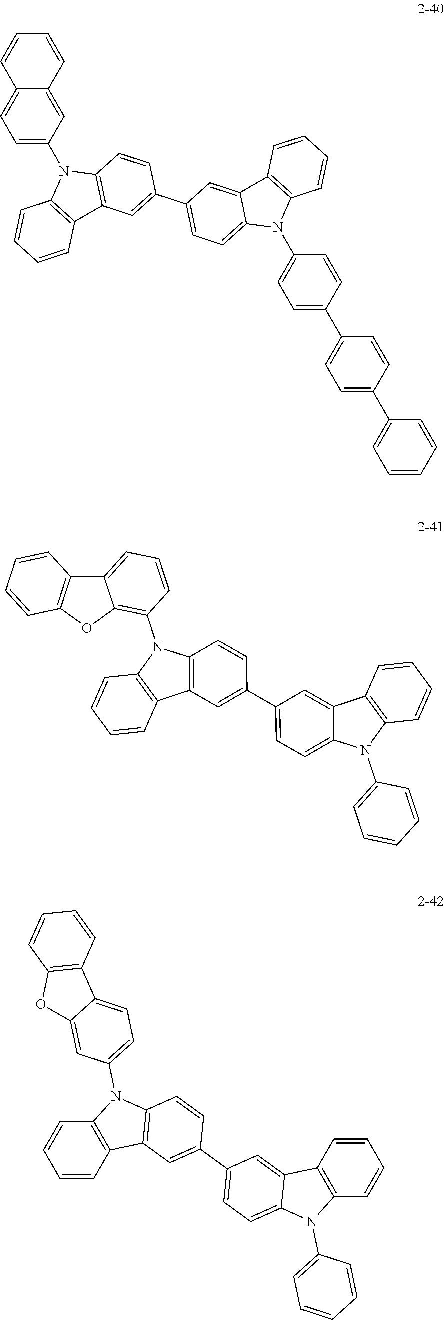 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4ccc5c(c4)oc4ccccc45)ccc32)cc1]

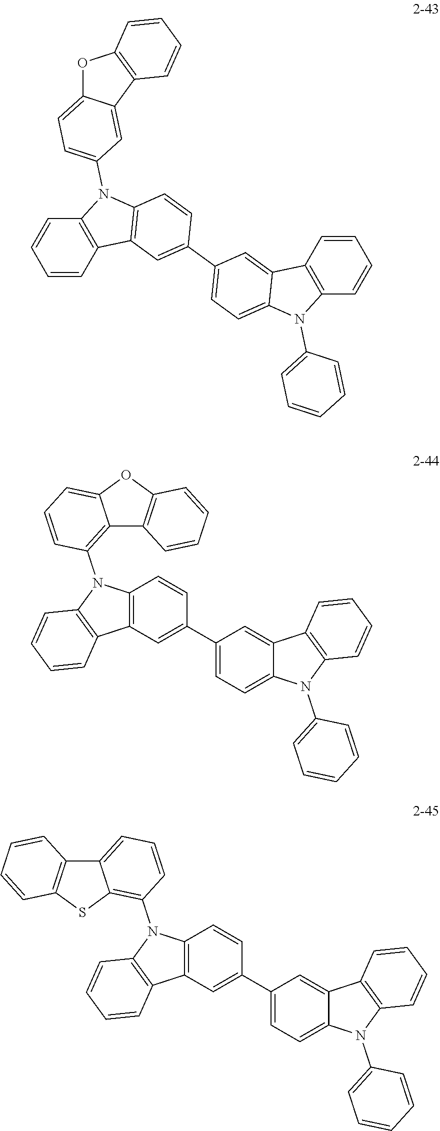 [SMILES: c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4sc4ccccc45)ccc32)cc1]

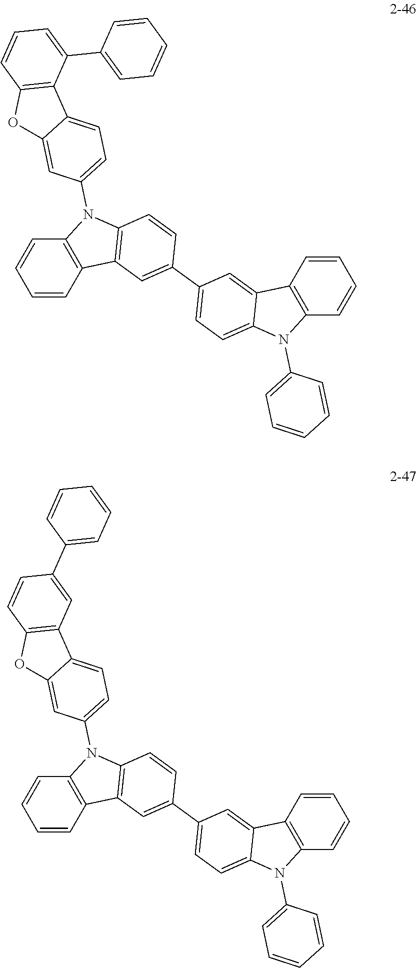 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc3oc4cc(-n5c6ccccc6c6cc(-c7ccc8c(c7)c7ccccc7n8-c7ccccc7)ccc65)ccc4c3c2)cc1]

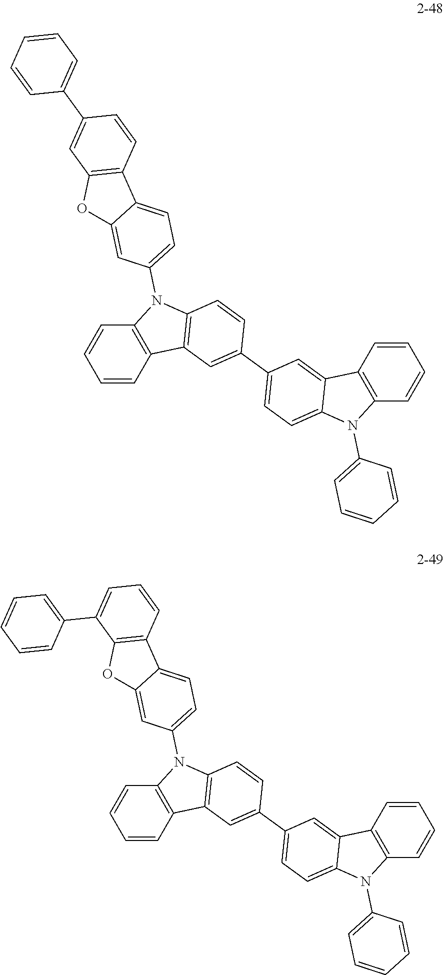 [SMILES: c1ccc(-c2ccc3c(c2)oc2cc(-n4c5ccccc5c5cc(-c6ccc7c(c6)c6ccccc6n7-c6ccccc6)ccc54)ccc23)cc1 ; c1ccc(-c2cccc3c2oc2cc(-n4c5ccccc5c5cc(-c6ccc7c(c6)c6ccccc6n7-c6ccccc6)ccc54)ccc23)cc1]

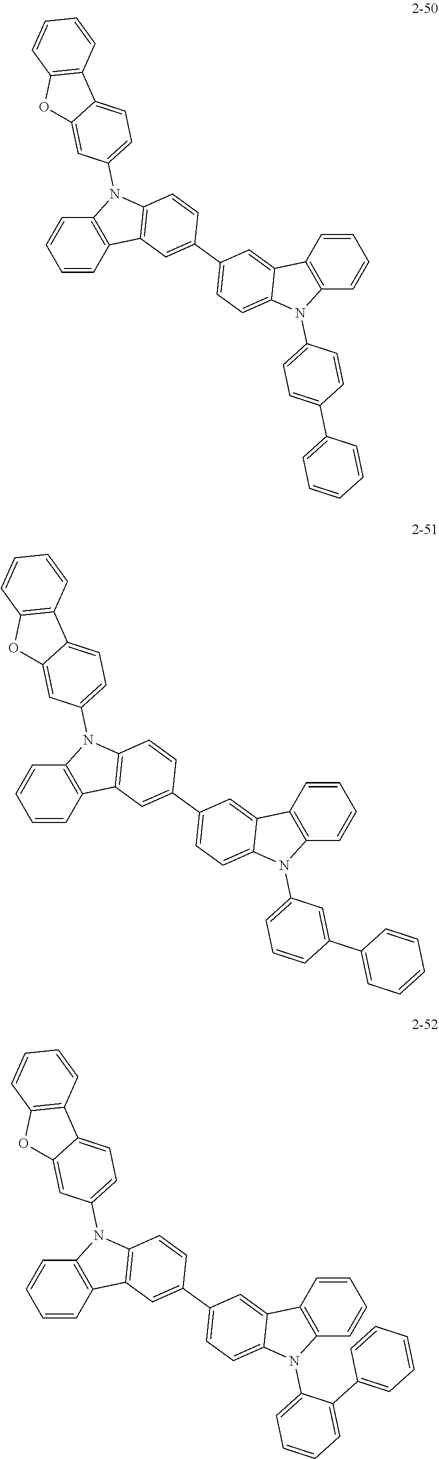 [SMILES: c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2ccccc2-n2c3ccccc3c3cc(-c4ccc5c(c4)c4ccccc4n5-c4ccc5c(c4)oc4ccccc45)ccc32)cc1]

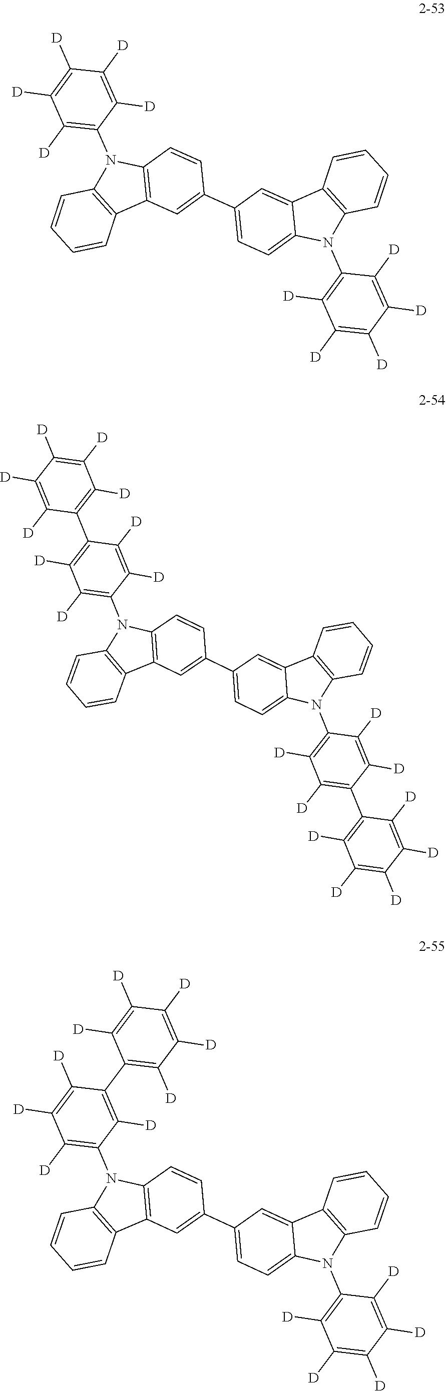 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]]

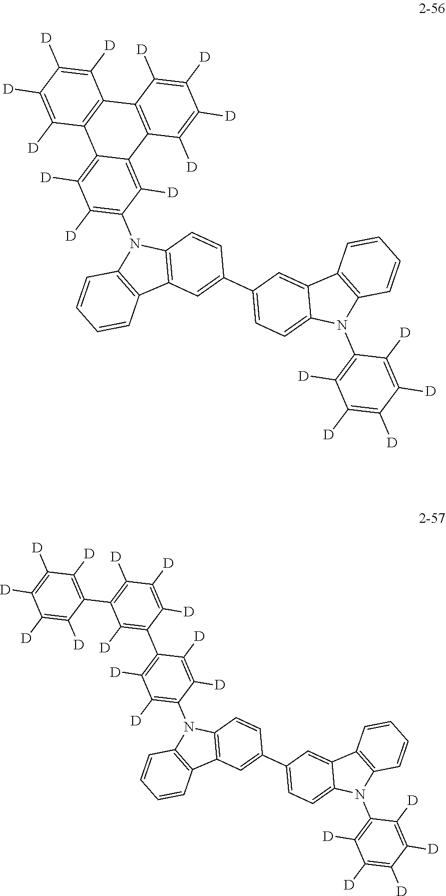 [SMILES: [2H]c1c([2H])c([2H])c(-n2c3ccccc3c3cc(-c4ccc5c(c4)c4ccccc4n5-c4c([2H])c([2H])c5c6c([2H])c([2H])c([2H])c([2H])c6c6c([2H])c([2H])c([2H])c([2H])c6c5c4[2H])ccc32)c([2H])c1[2H] ; [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c2[2H])c([2H])c1[2H]]

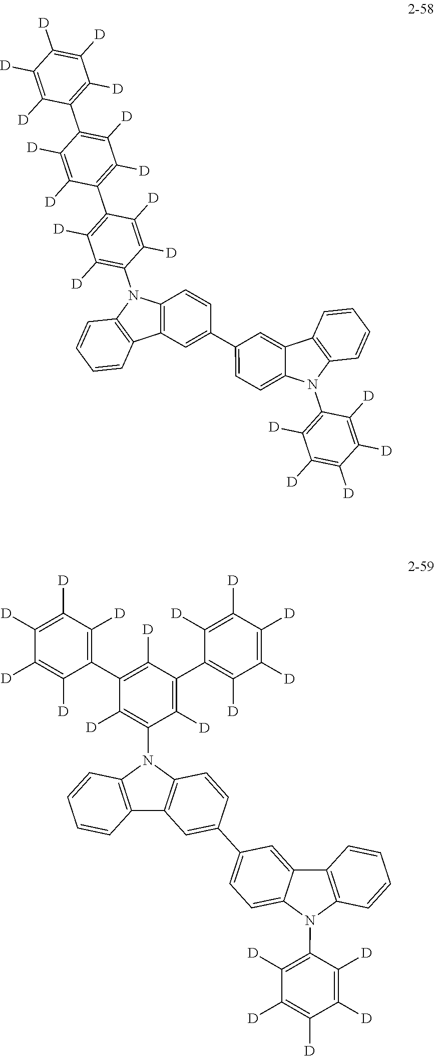 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c([2H])c2[2H])c([2H])c1[2H] ; [2H]c1c([2H])c([2H])c(-c2c([2H])c(-c3c([2H])c([2H])c([2H])c([2H])c3[2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]]

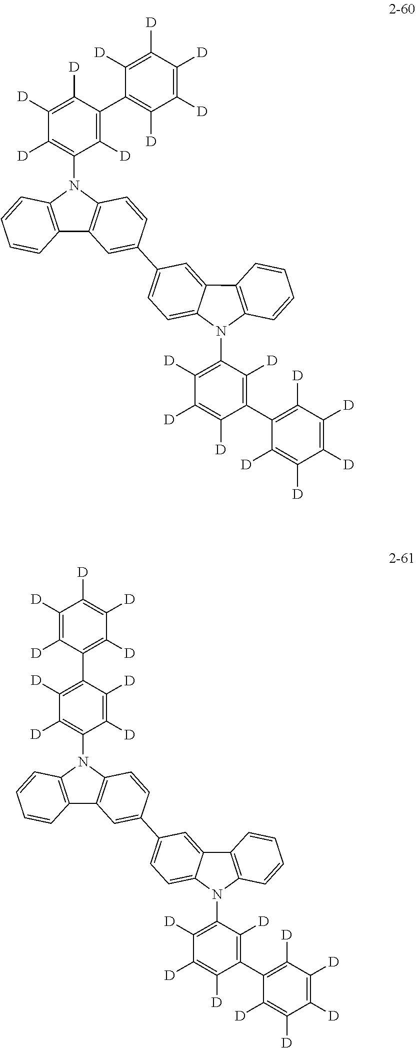 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]]

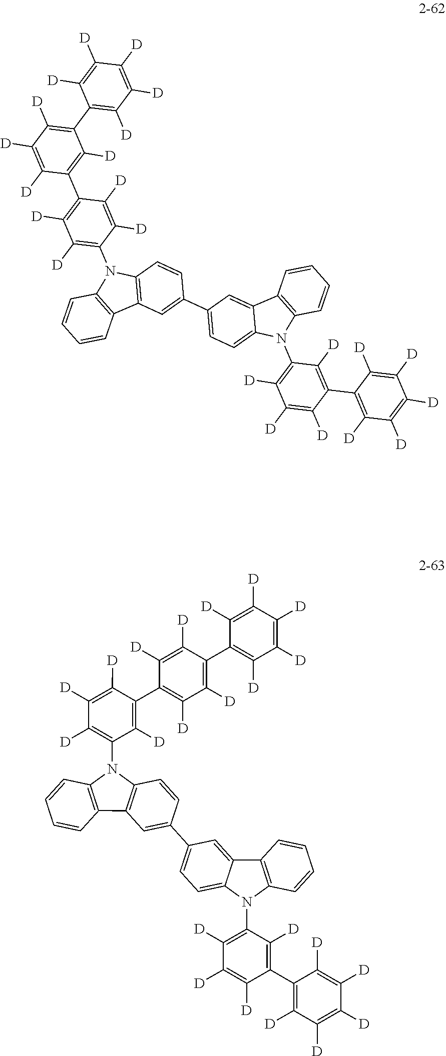 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c3[2H])c([2H])c2[2H])c([2H])c1[2H]]

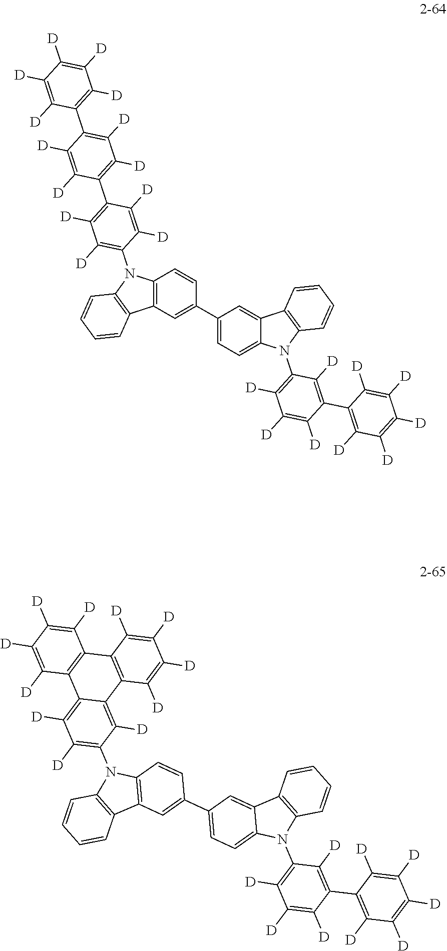 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H]]

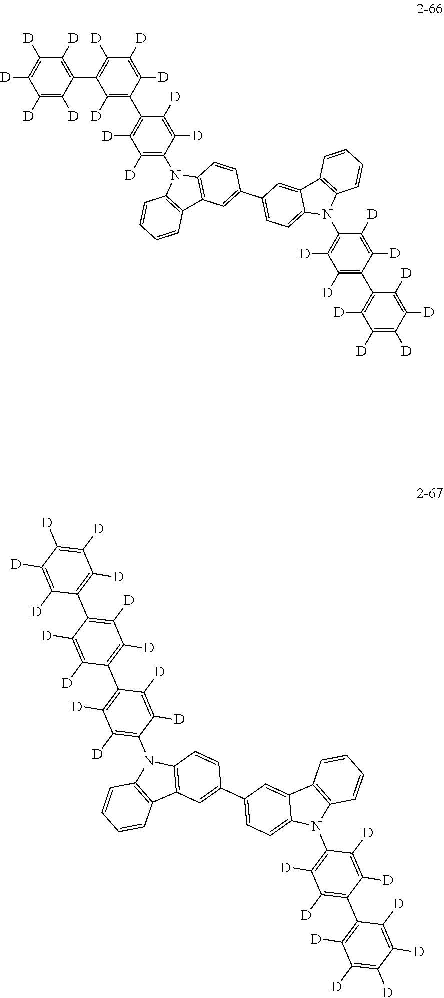 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H] ; [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H]]

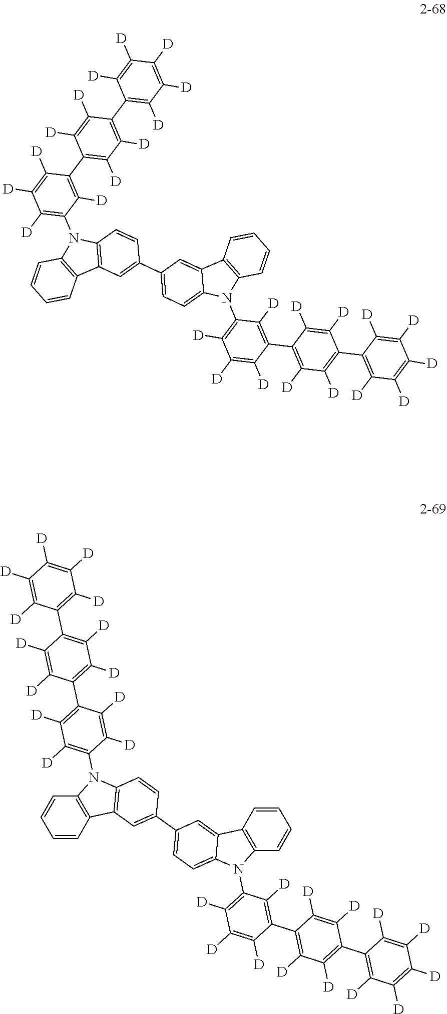

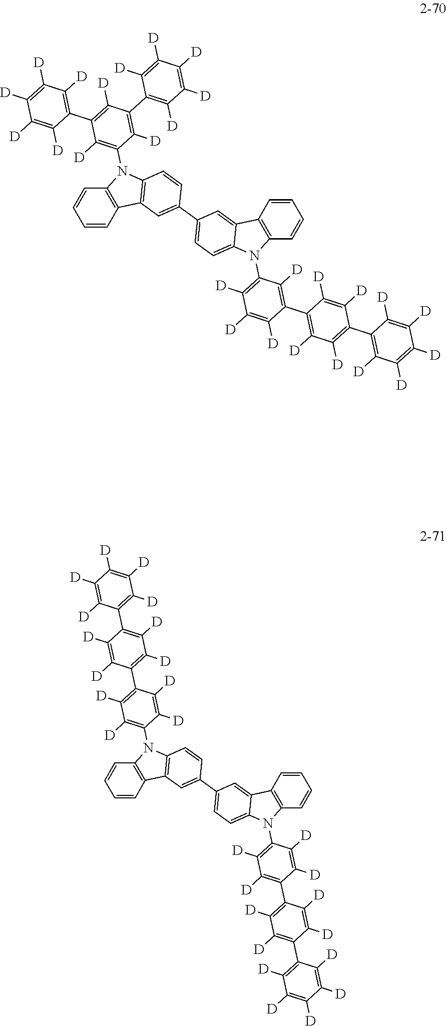

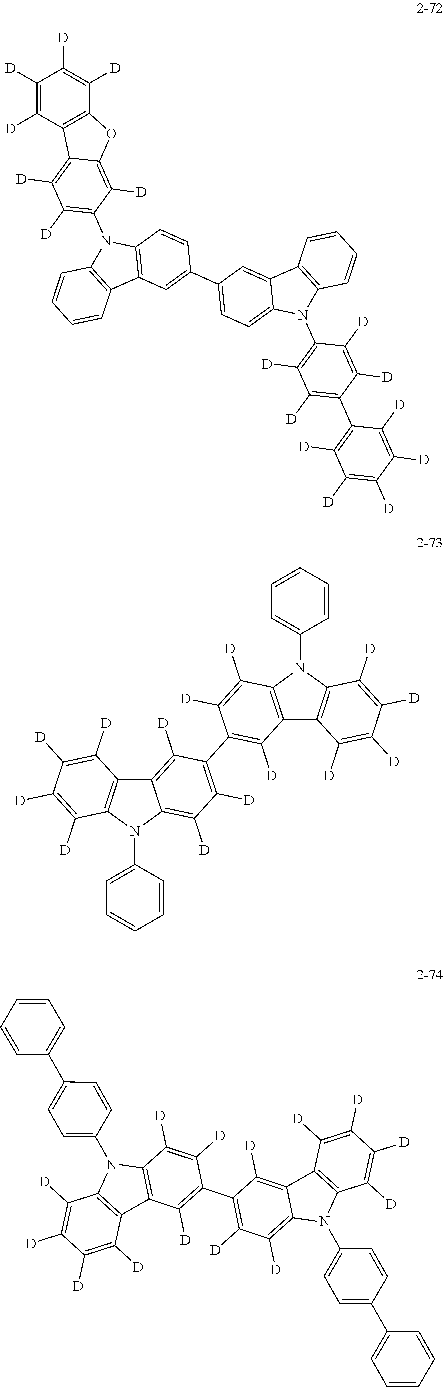 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

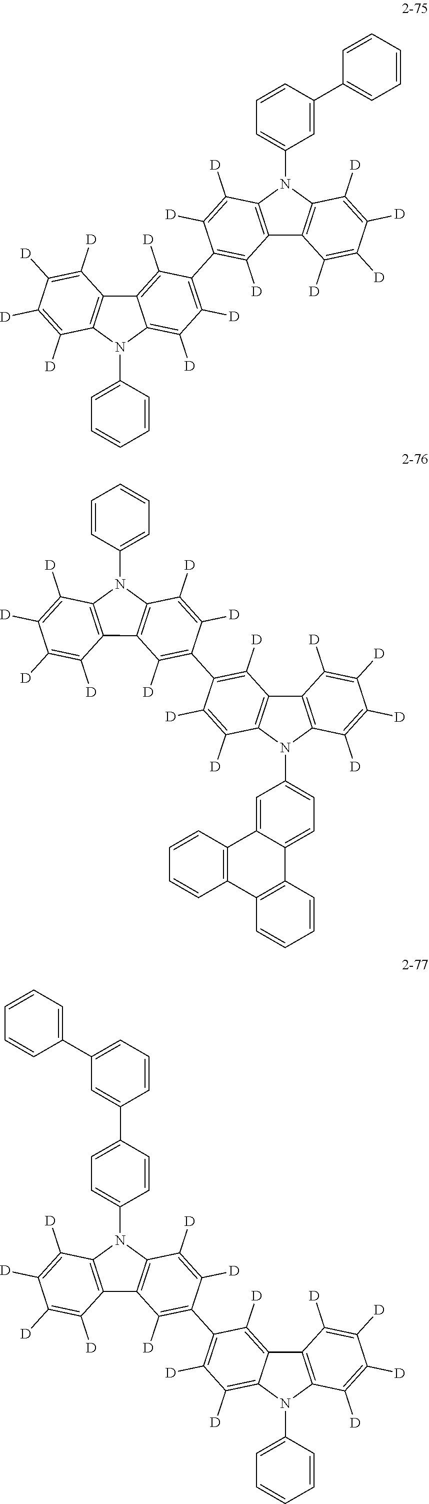

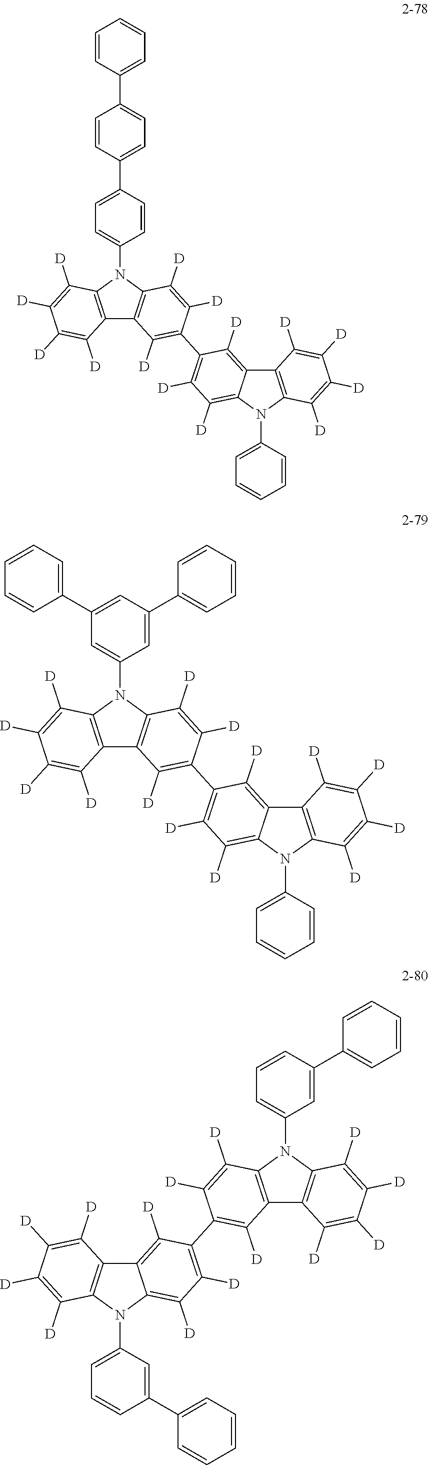 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1cccc(-c2ccccc2)c1]

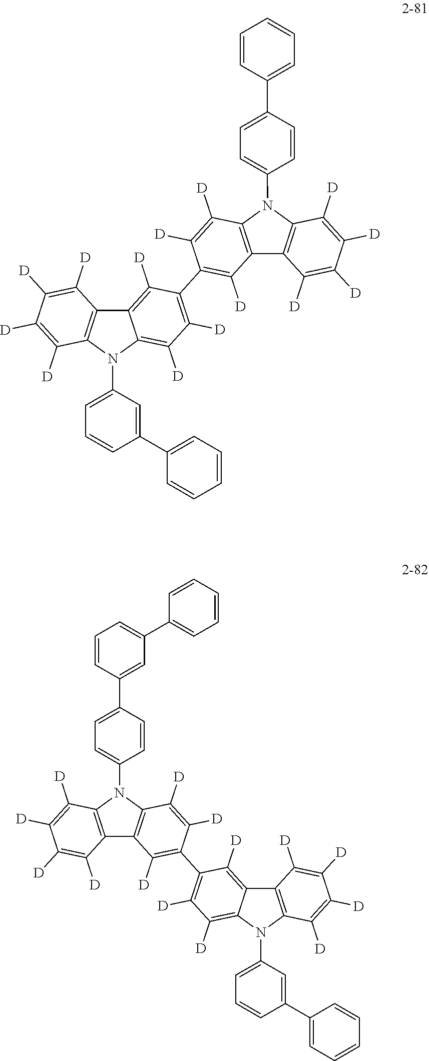 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccccc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2cccc(-c3ccccc3)c2)cc1]

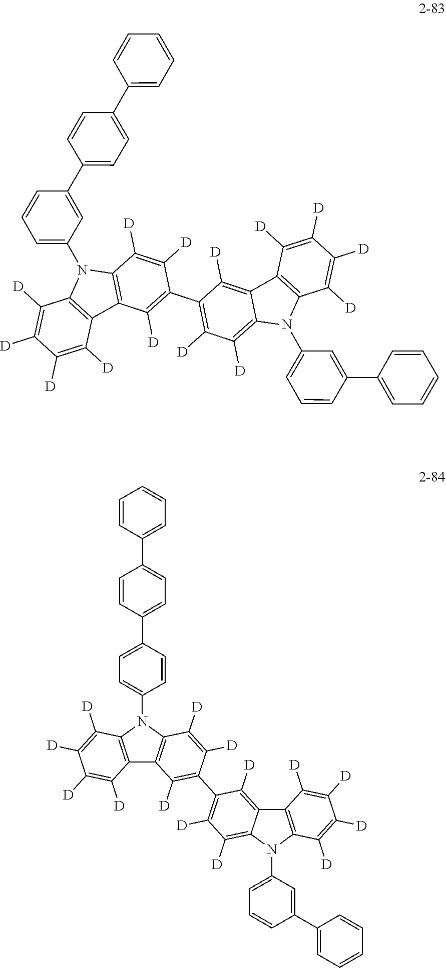 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1cccc(-c2ccccc2)c1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccc(-c3ccccc3)cc2)cc1]

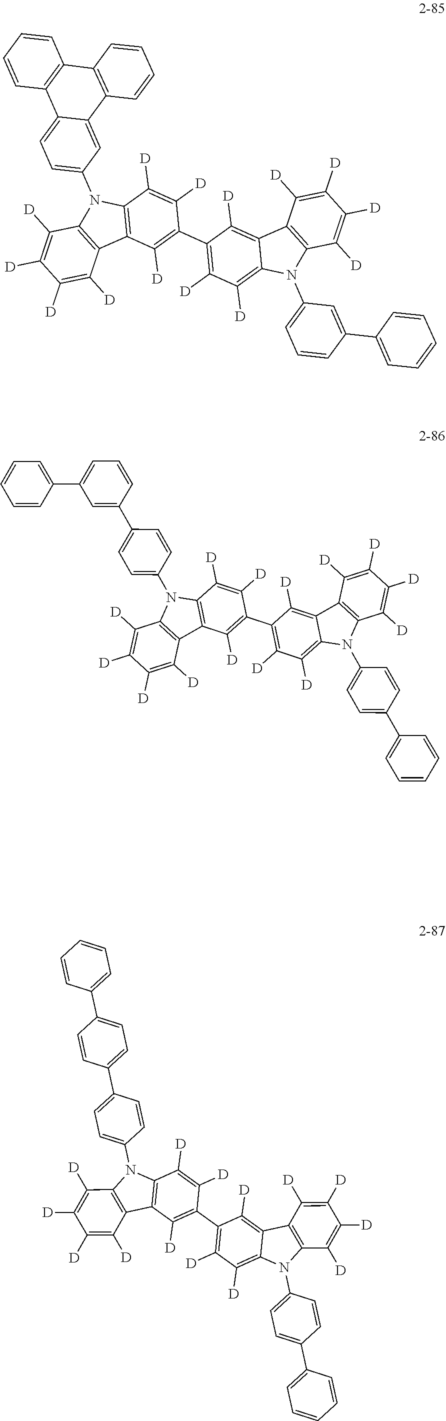

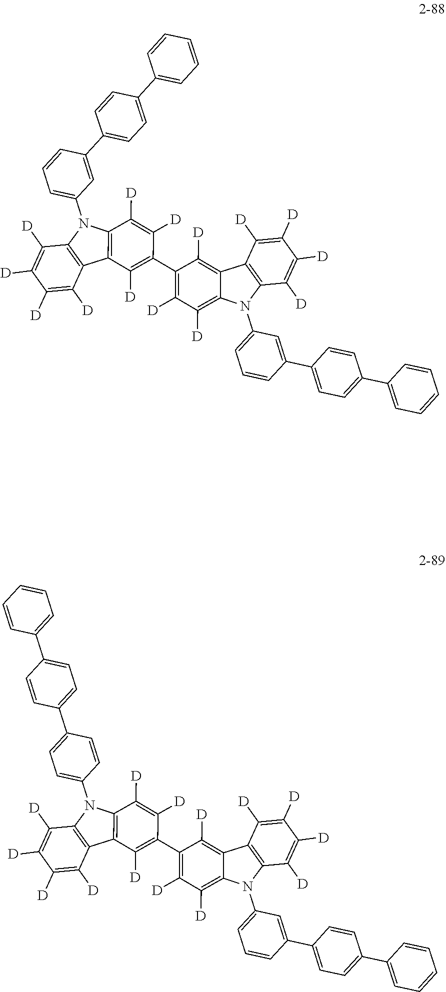 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1cccc(-c2ccc(-c3ccccc3)cc2)c1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1ccc(-c2ccc(-c3ccccc3)cc2)cc1]

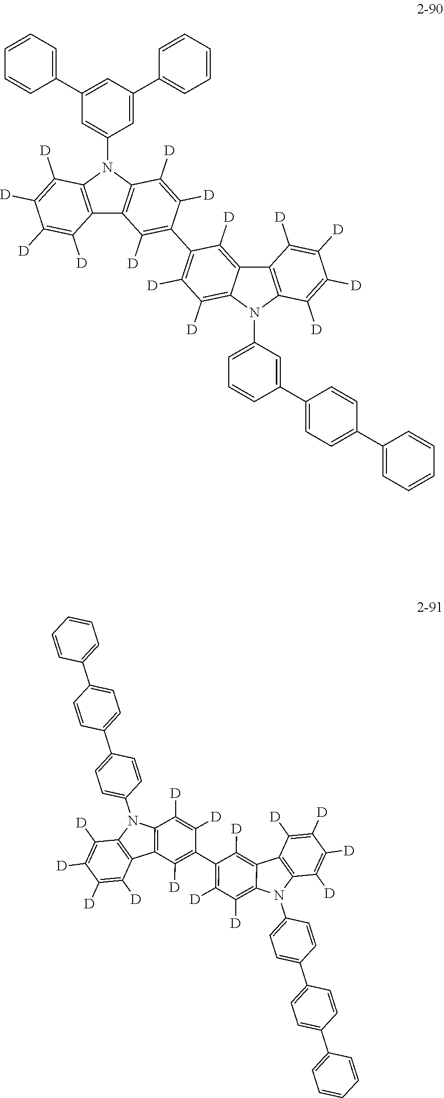 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cccc(-c2ccc(-c3ccccc3)cc2)c1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccc(-c3ccccc3)cc2)cc1]

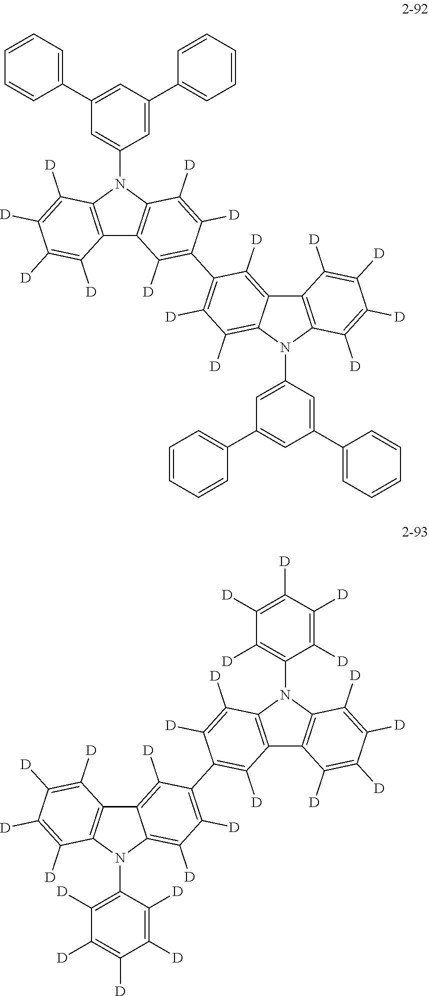 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cc(-c2ccccc2)cc(-c2ccccc2)c1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

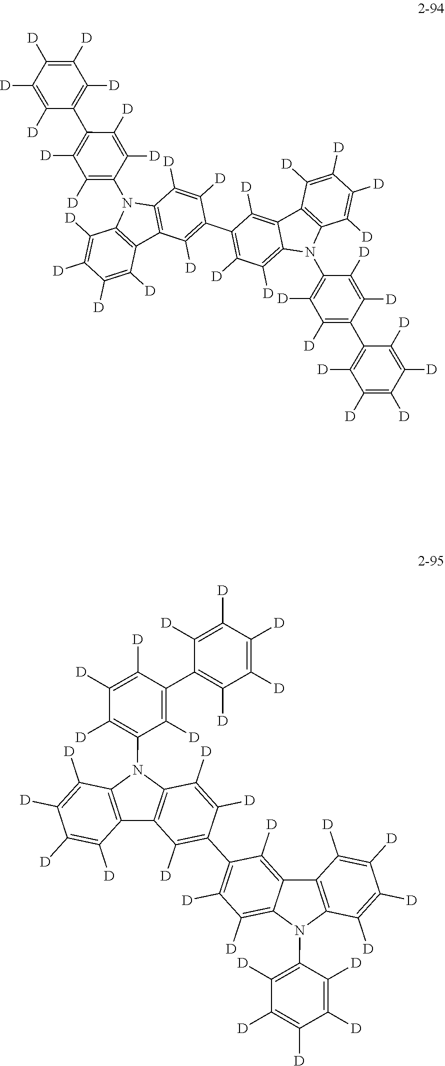 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]]

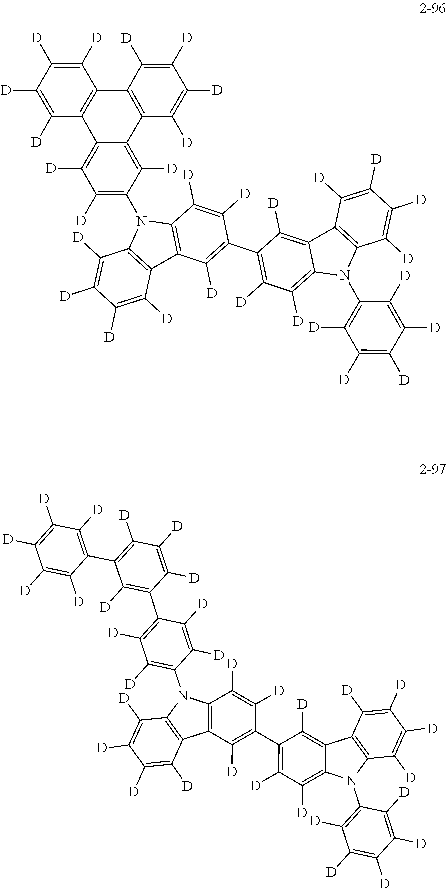

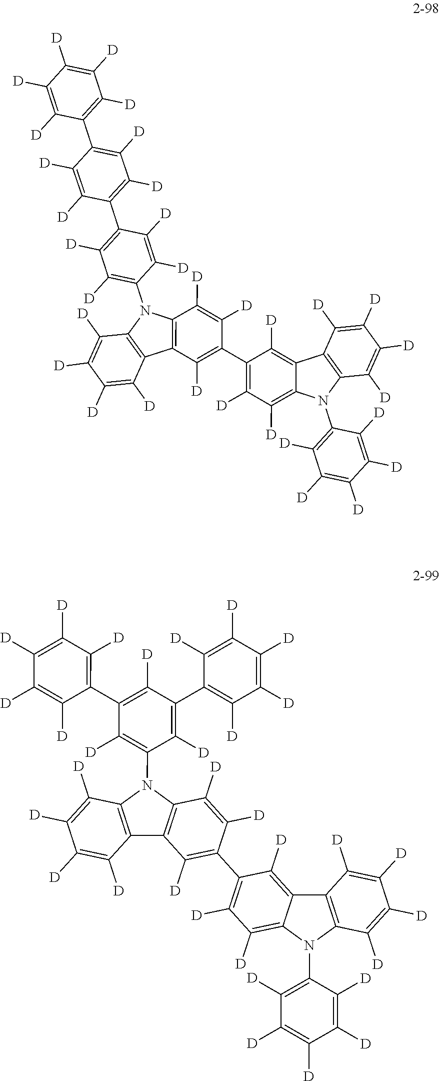

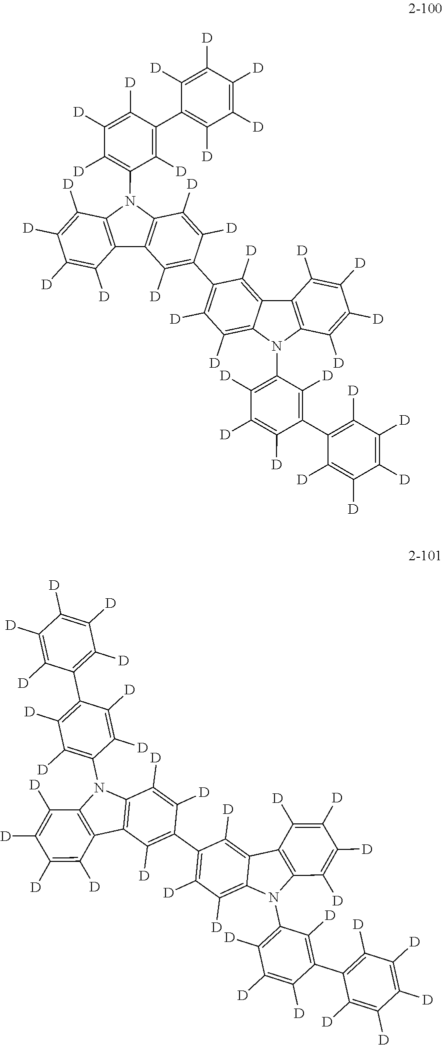

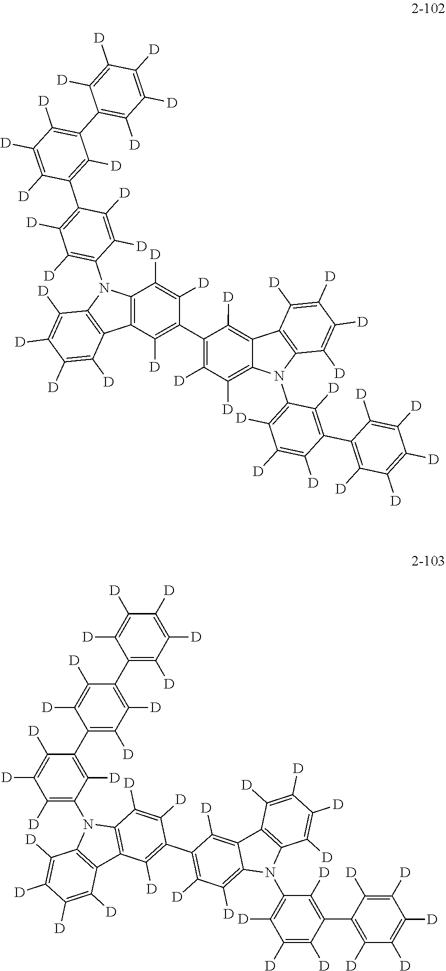

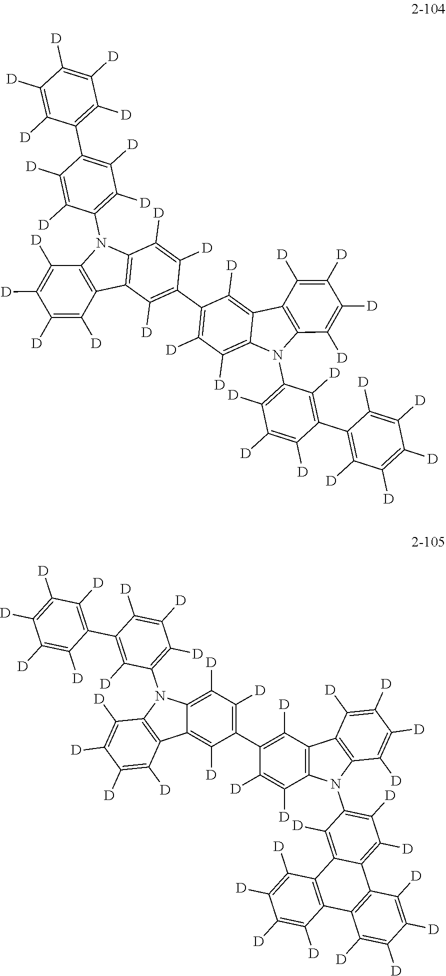

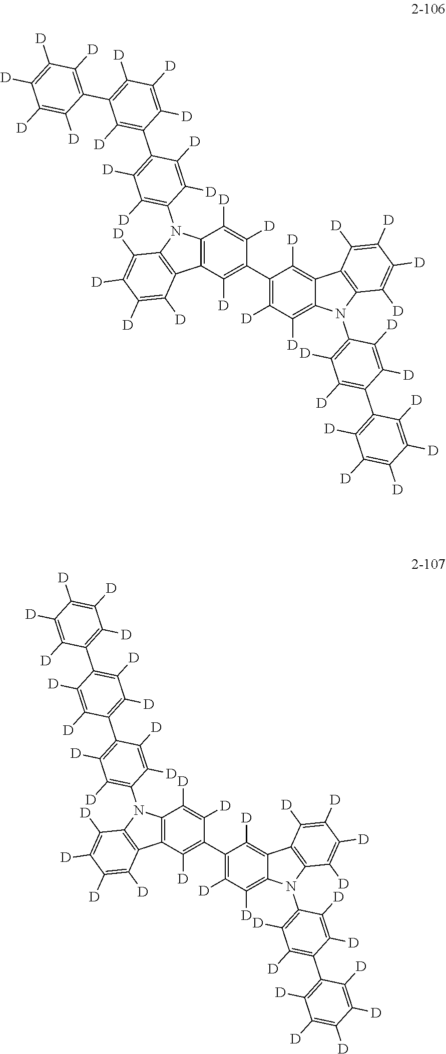

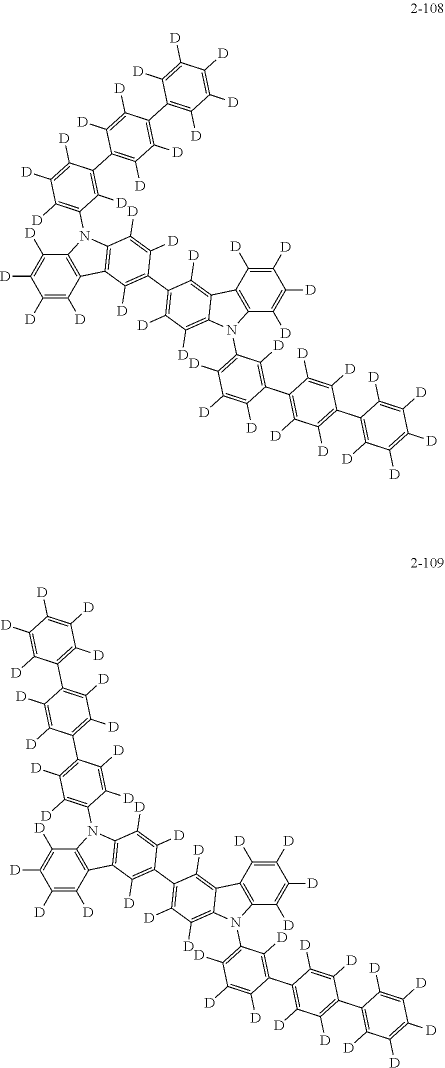

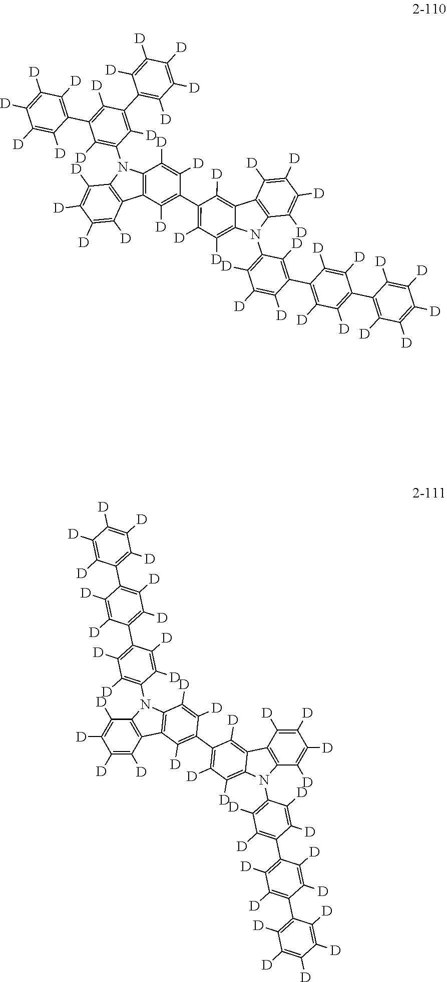

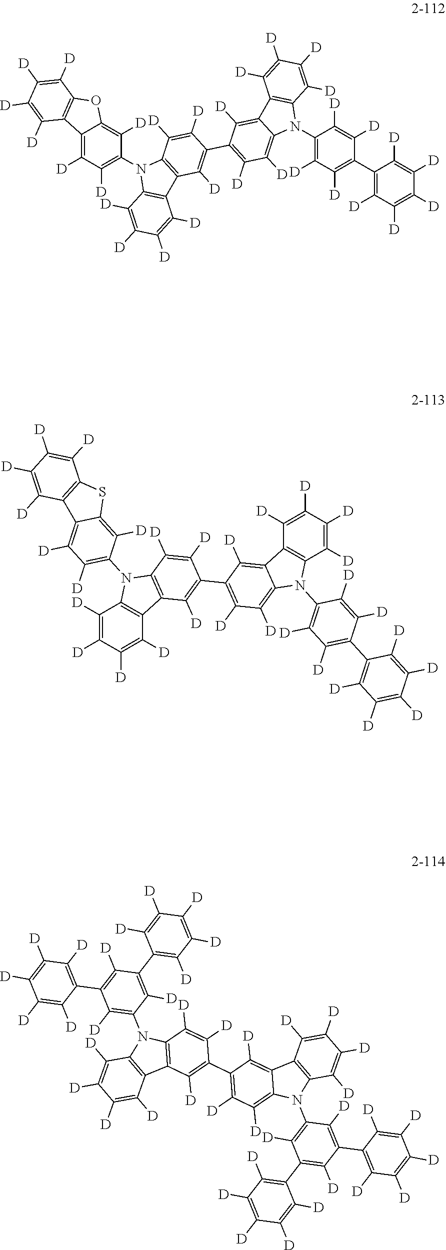

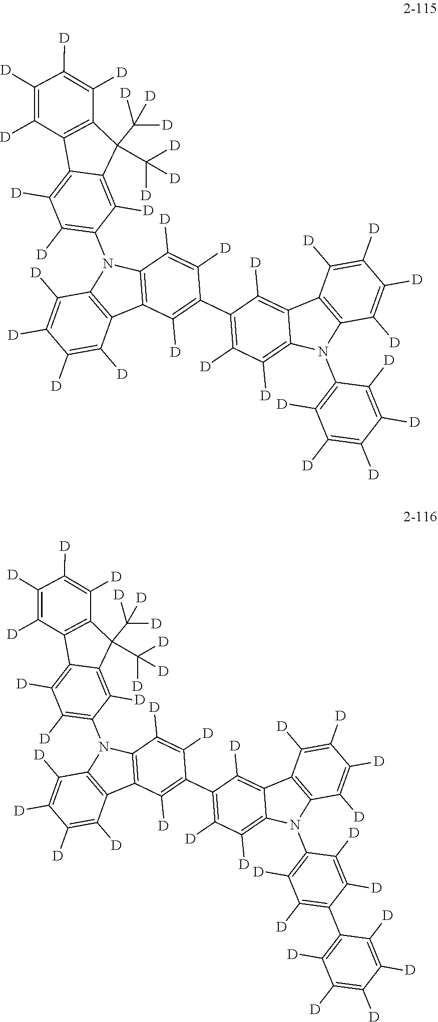

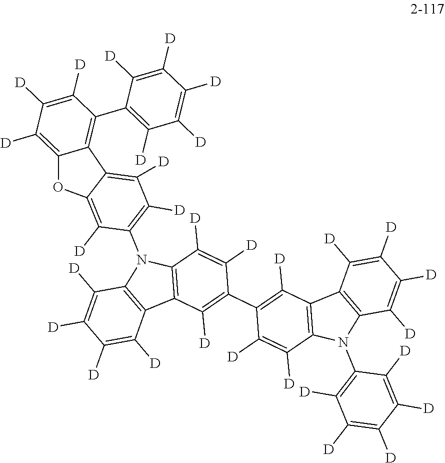 [SMILES: [2H]c1c([2H])c([2H])c(-c2c([2H])c([2H])c([2H])c3oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c4c23)c([2H])c1[2H]]

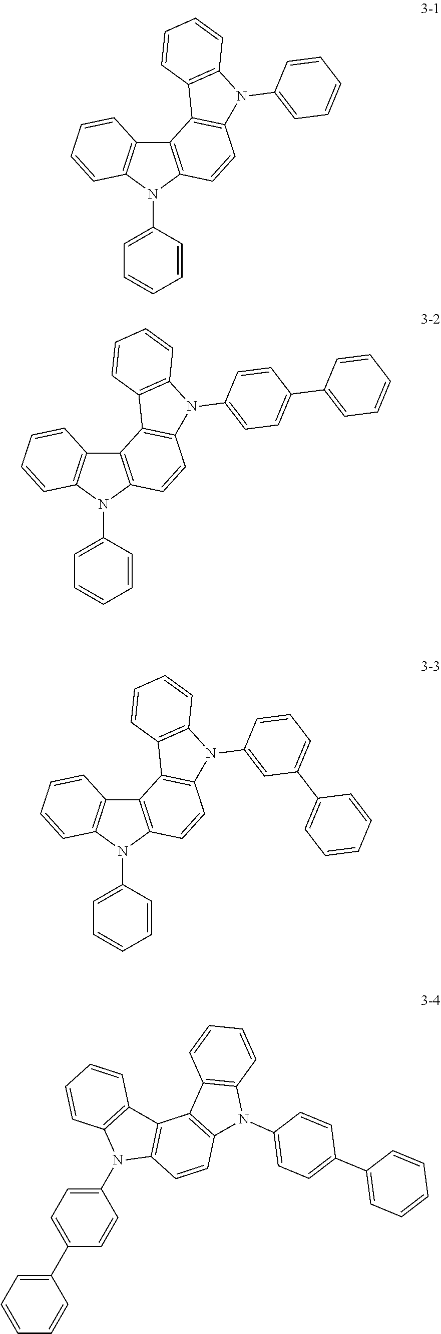

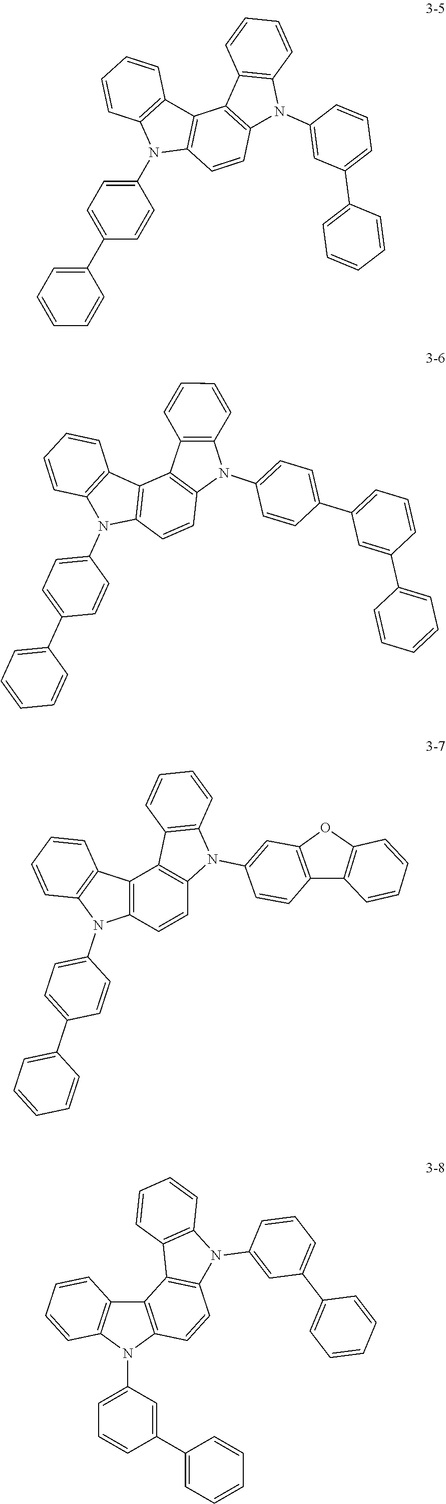 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

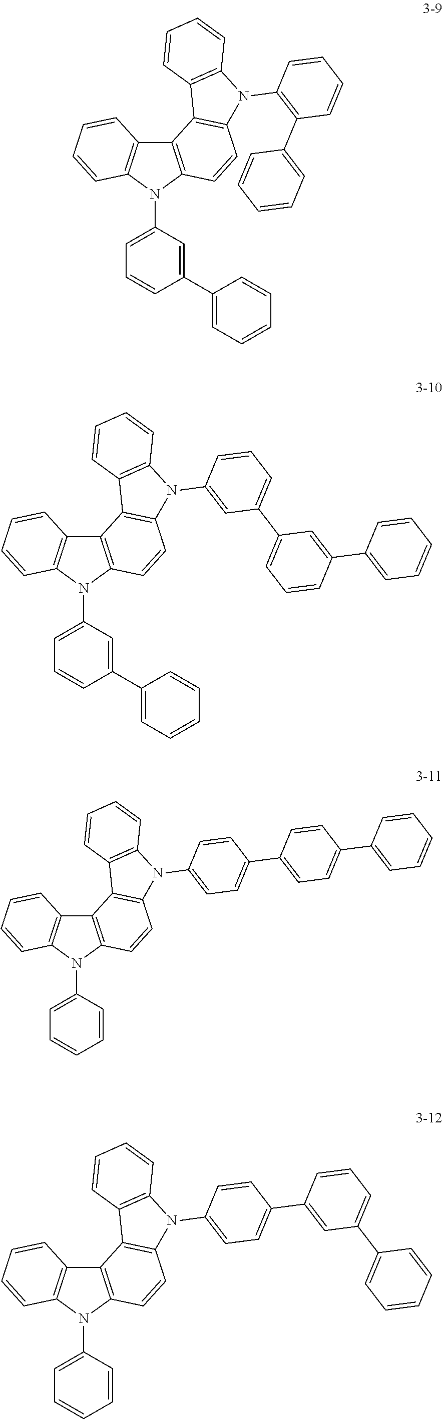 [SMILES: c1ccc(-c2cccc(-n3c4ccccc4c4c5c6ccccc6n(-c6ccccc6-c6ccccc6)c5ccc43)c2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1]

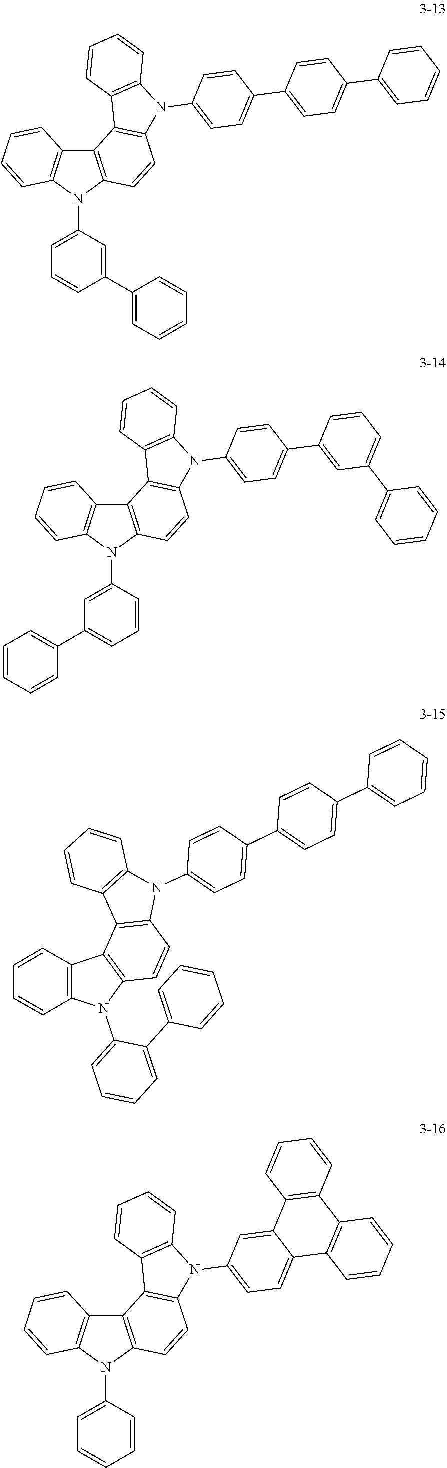 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

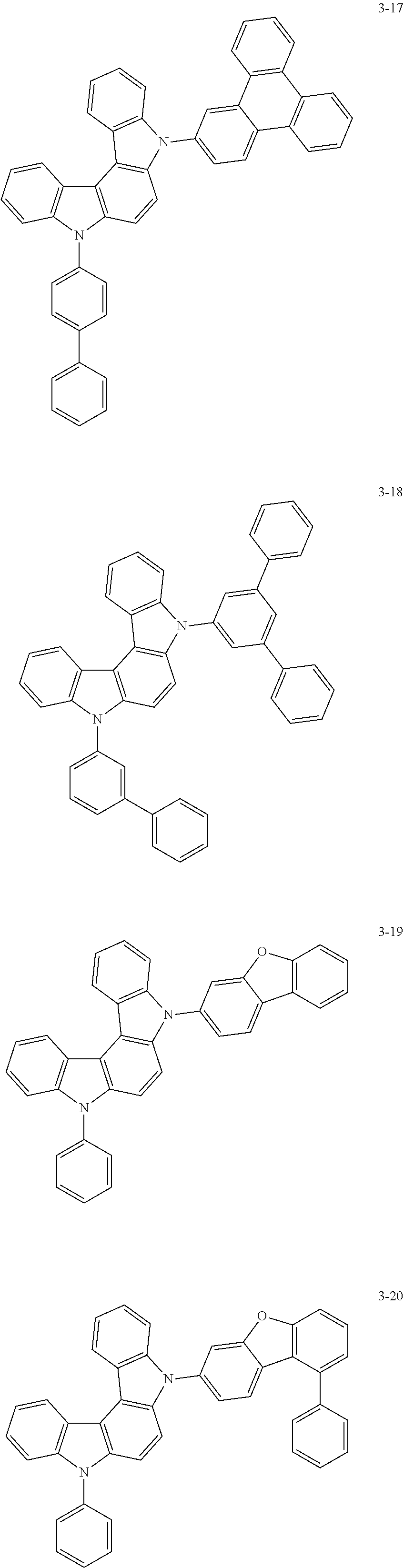 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1]

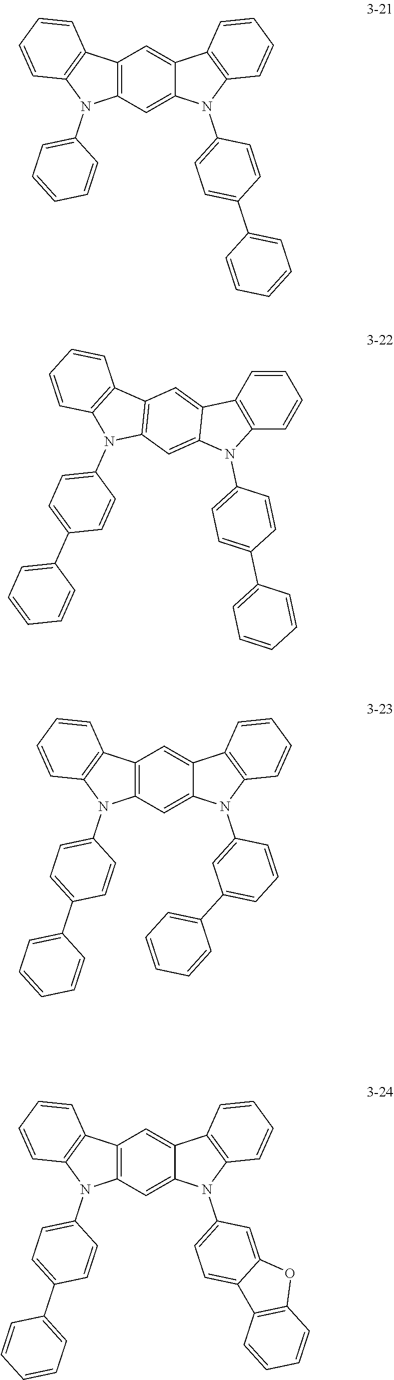 [SMILES: c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1]

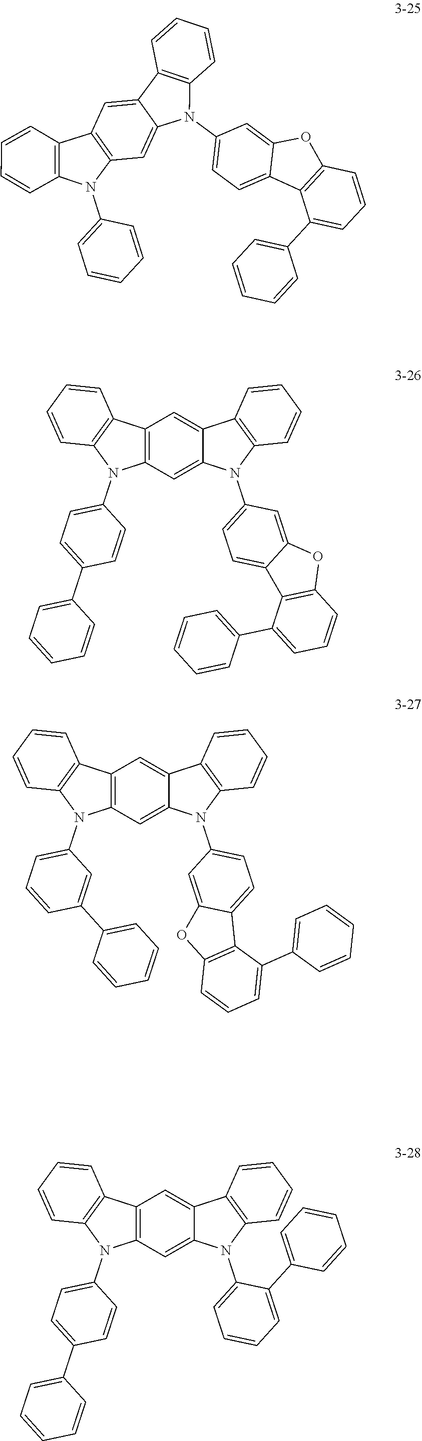 [SMILES: c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1 ; c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1]

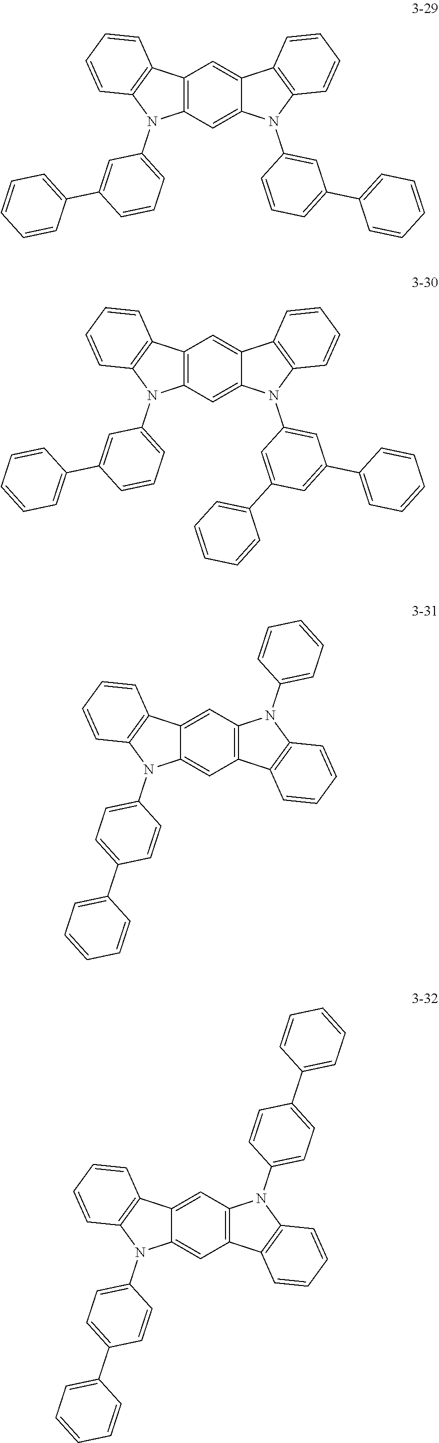 [SMILES: c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

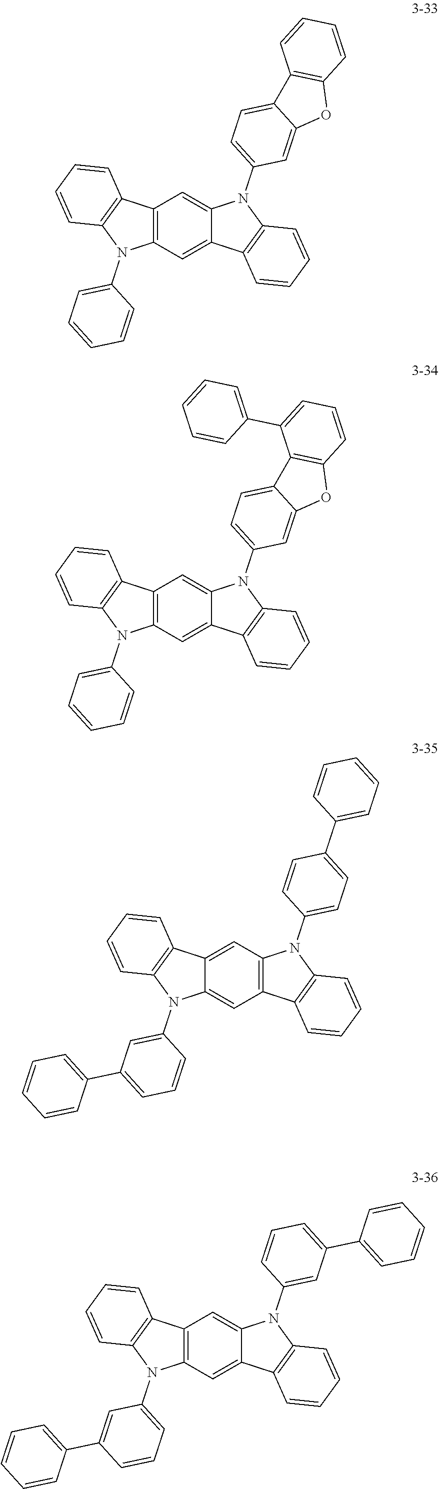 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1]

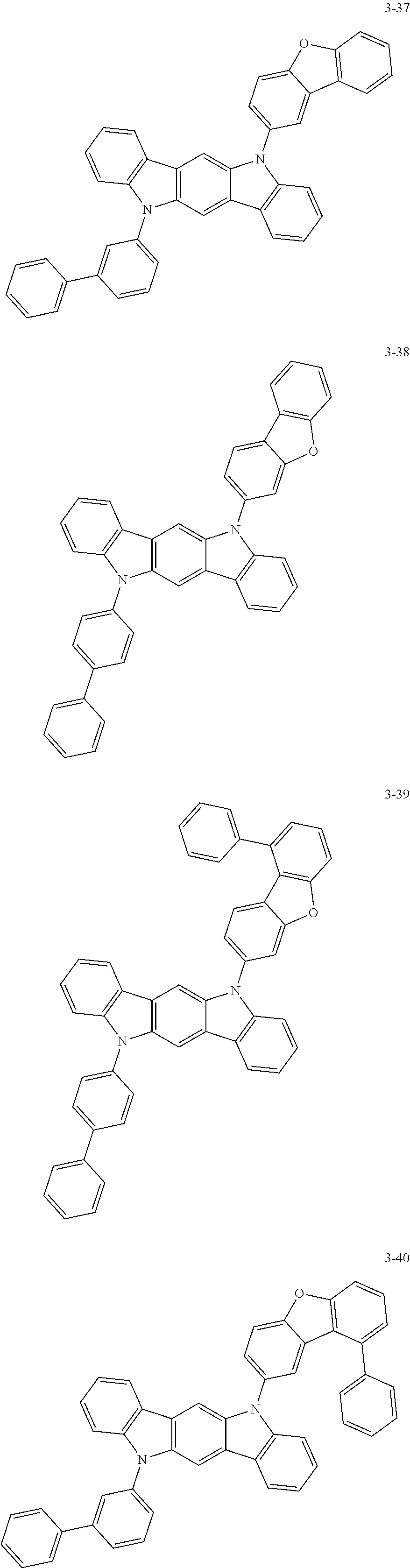 [SMILES: c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

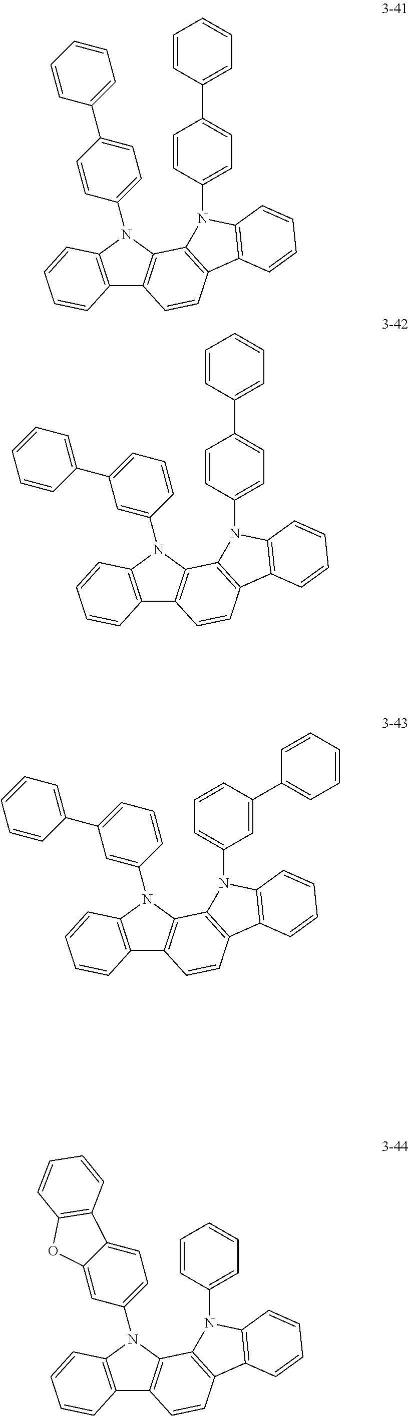 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

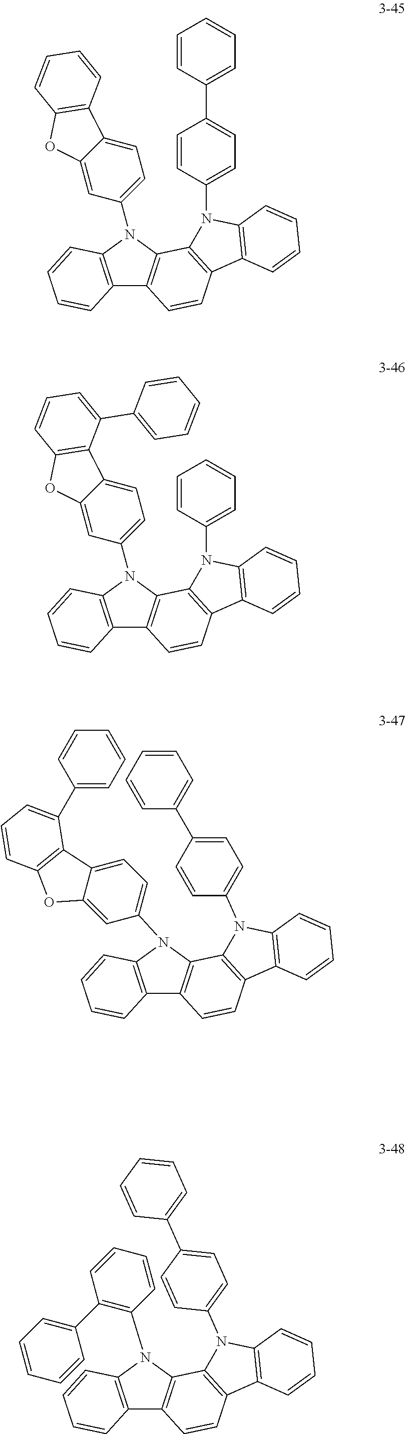 [SMILES: c1ccc(-c2ccc(-n3c4ccccc4c4ccc5c6ccccc6n(-c6ccc7c(c6)oc6ccccc67)c5c43)cc2)cc1 ; c1ccc(-c2cccc3oc4cc(-n5c6ccccc6c6ccc7c8ccccc8n(-c8ccccc8)c7c65)ccc4c23)cc1 ; c1ccc(-c2ccc(-n3c4ccccc4c4ccc5c6ccccc6n(-c6ccc7c(c6)oc6cccc(-c8ccccc8)c67)c5c43)cc2)cc1 ; c1ccc(-c2ccc(-n3c4ccccc4c4ccc5c6ccccc6n(-c6ccccc6-c6ccccc6)c5c43)cc2)cc1]

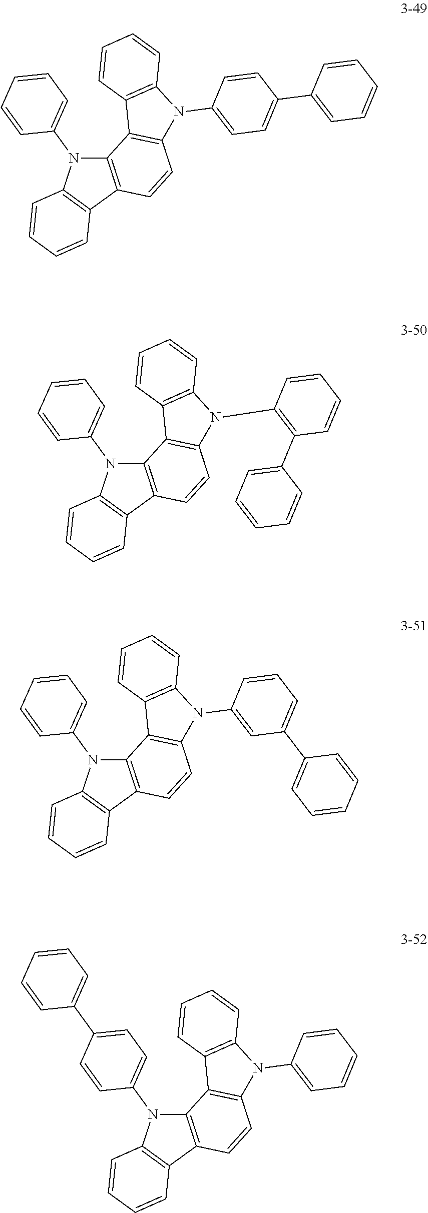 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

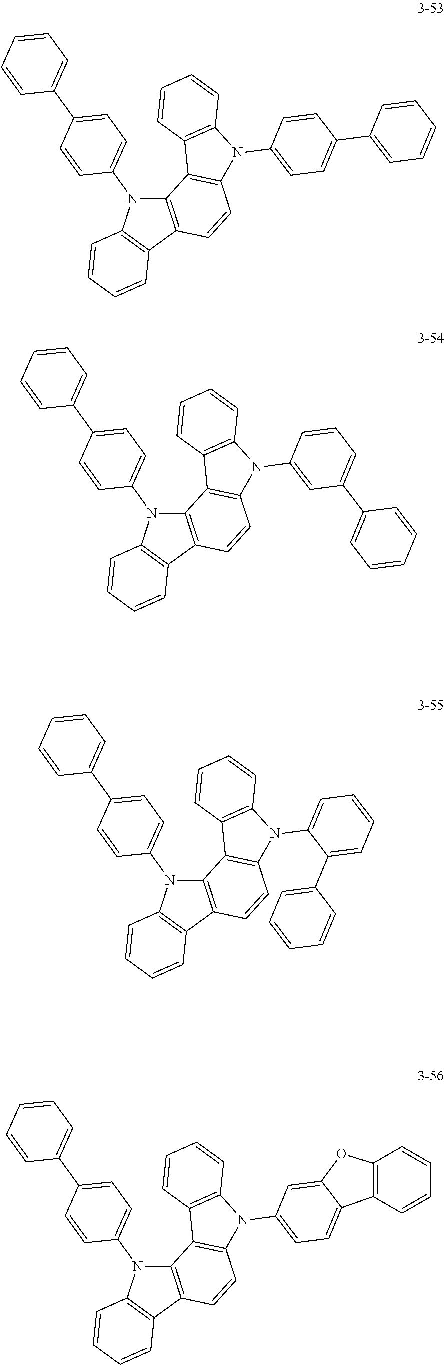 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1]

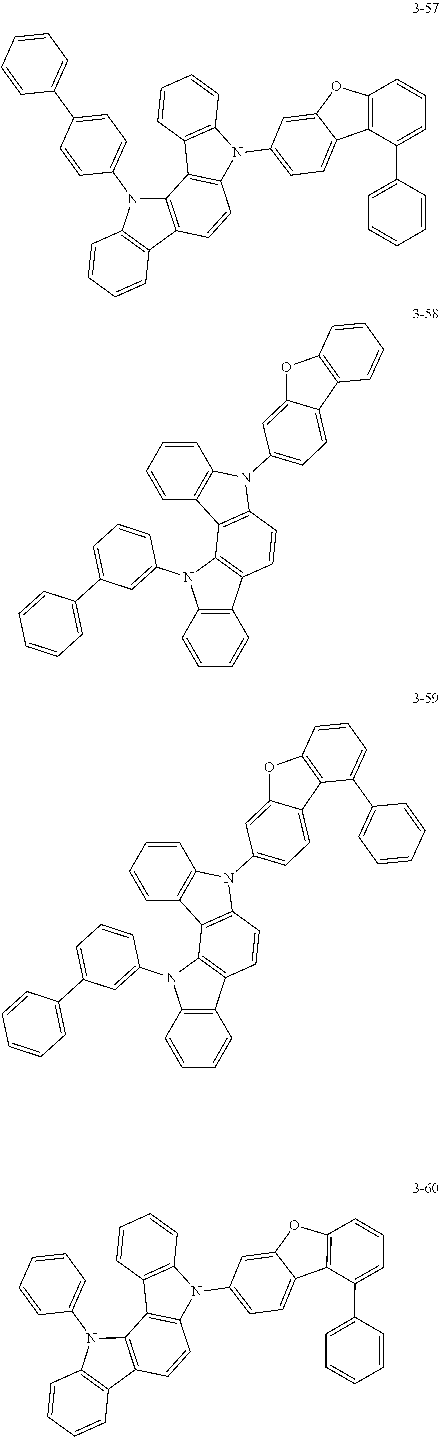 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

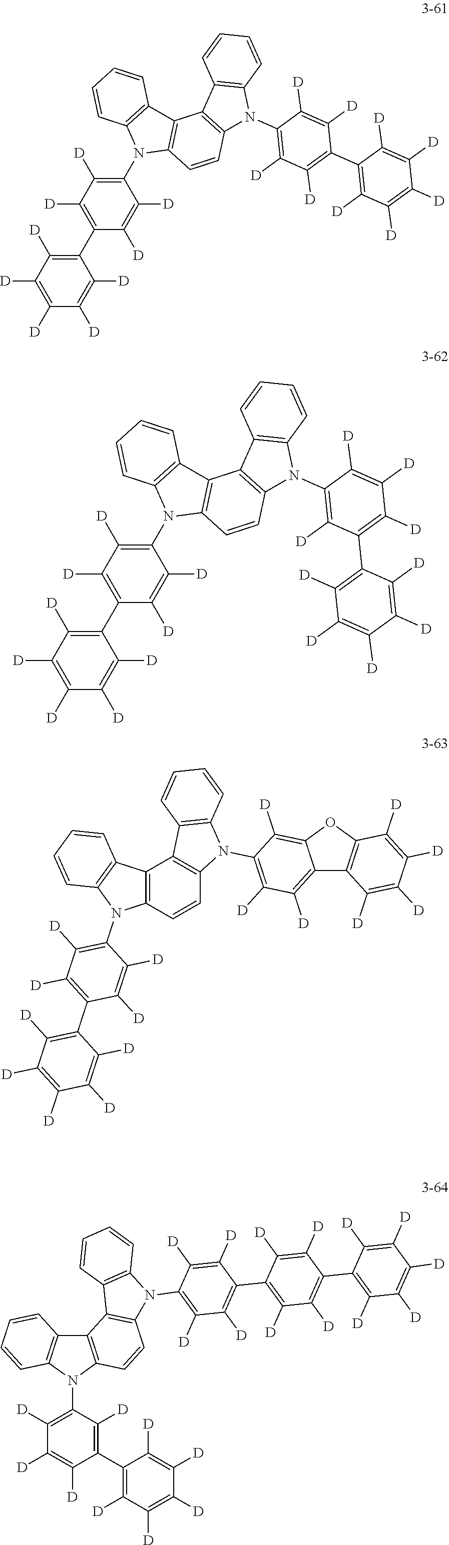

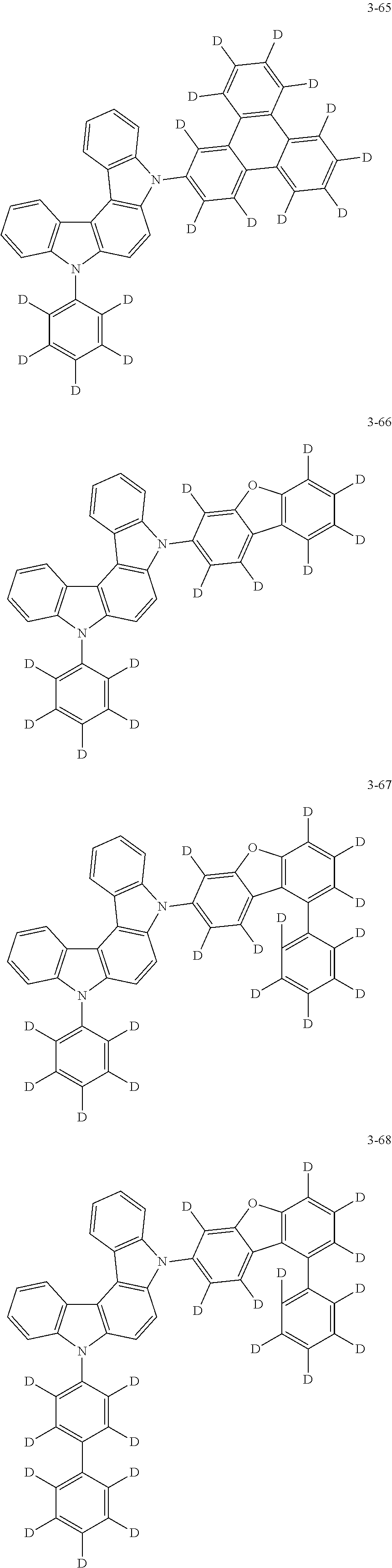

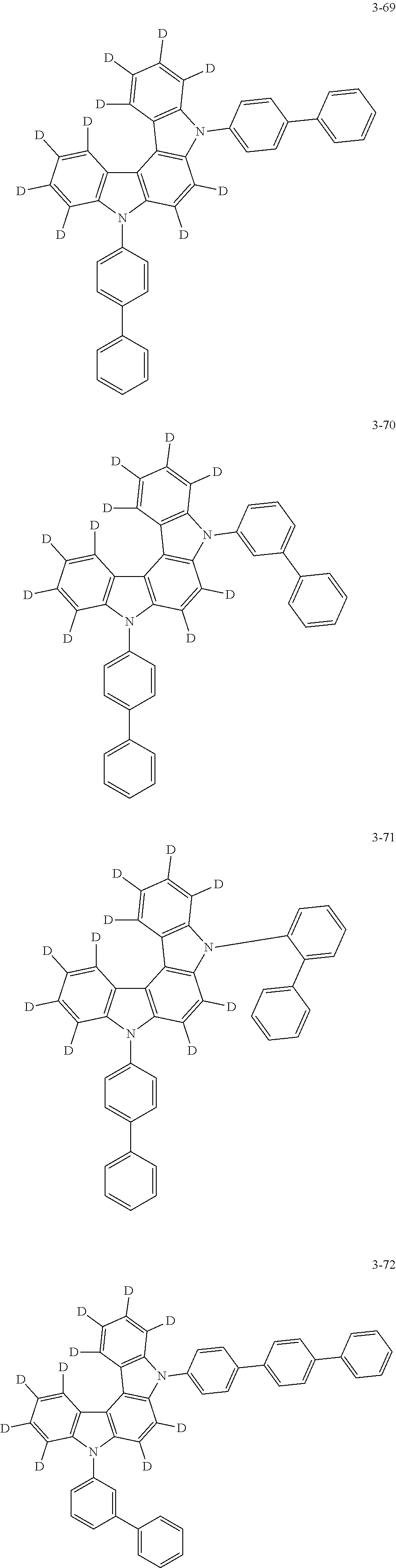

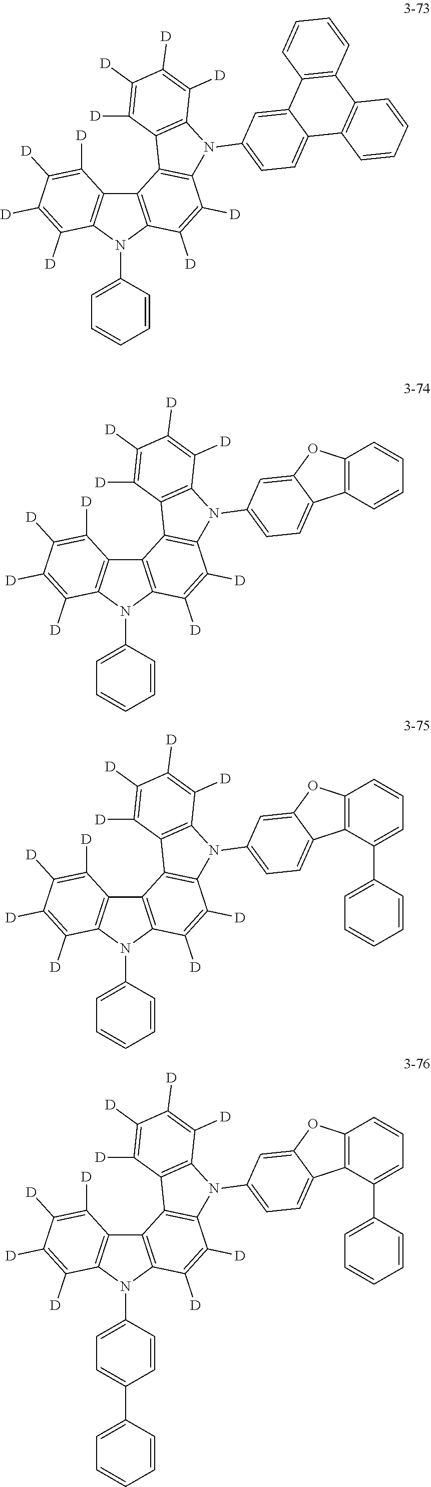

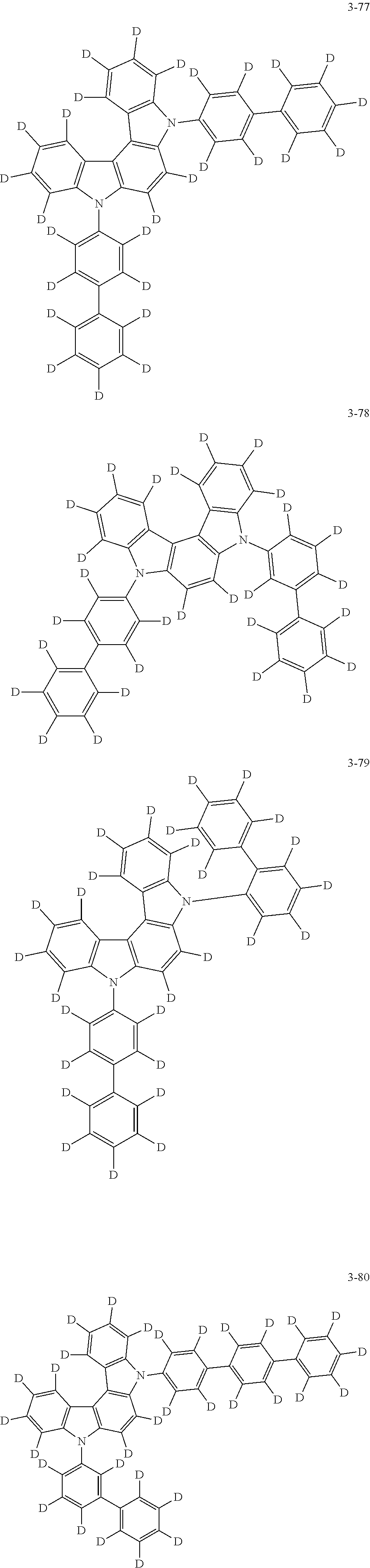

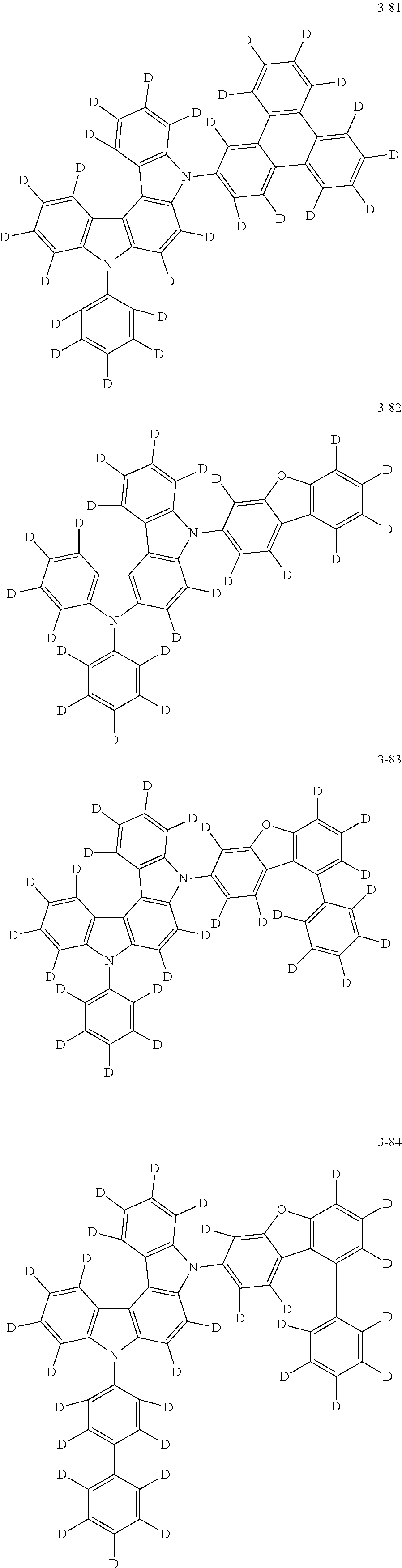

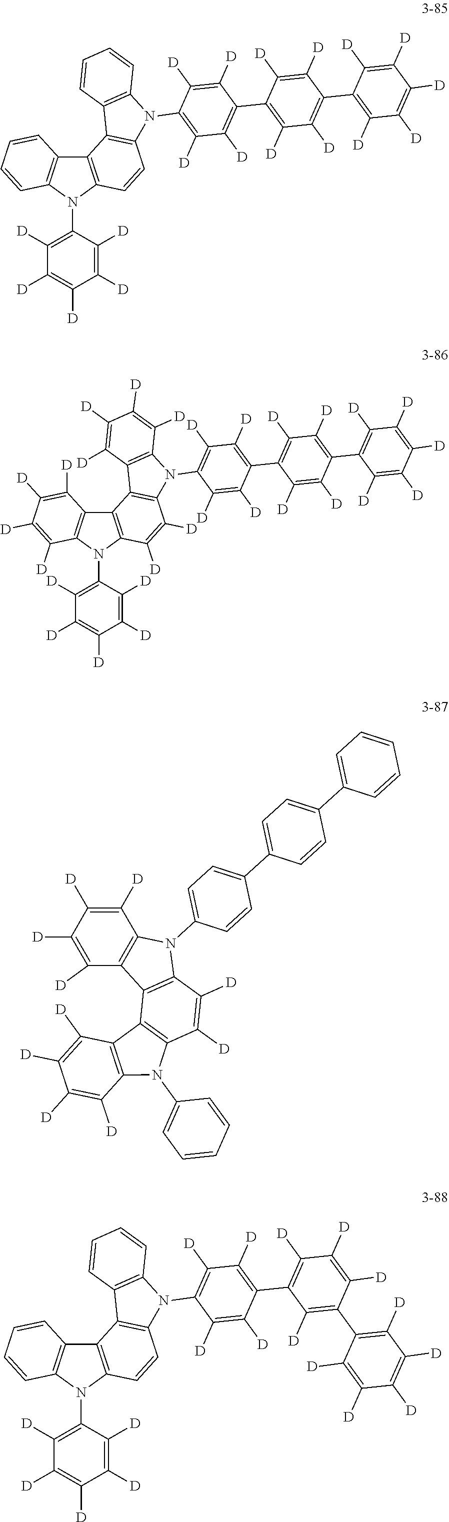

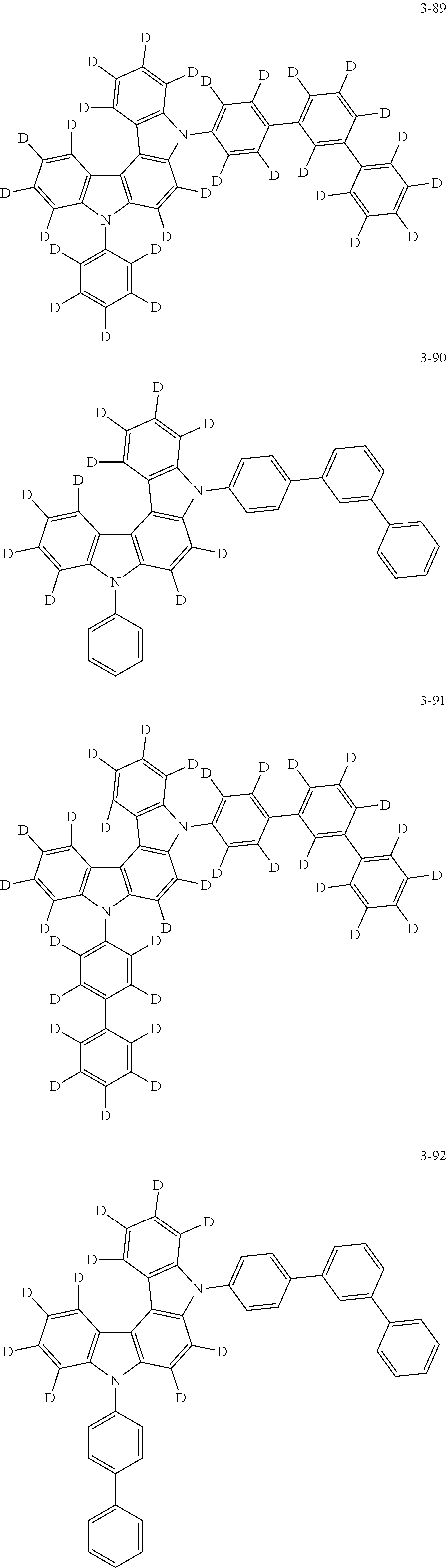

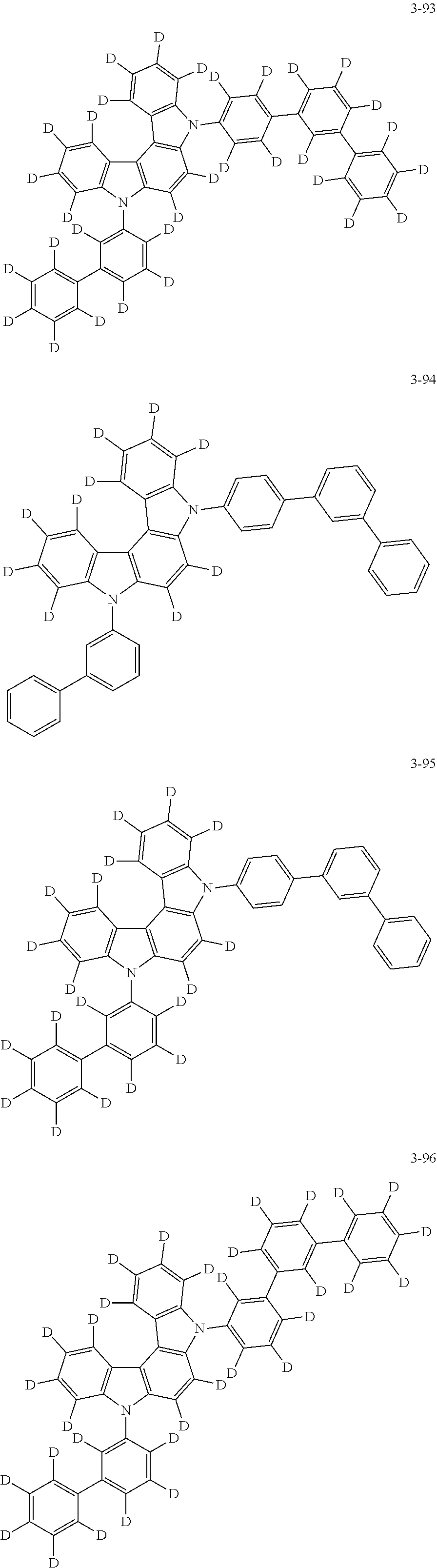

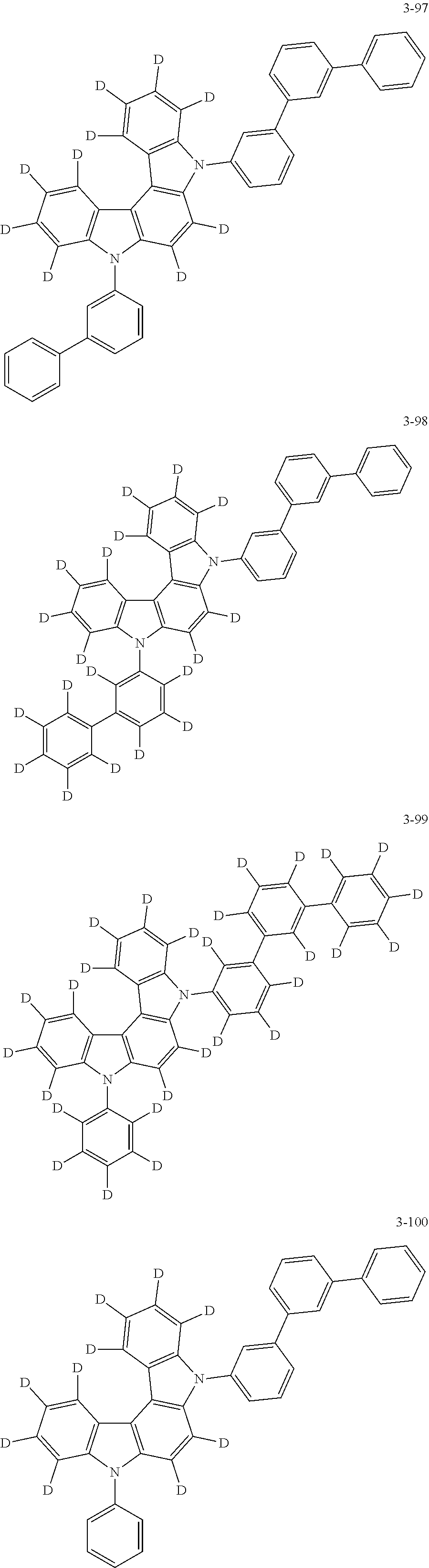

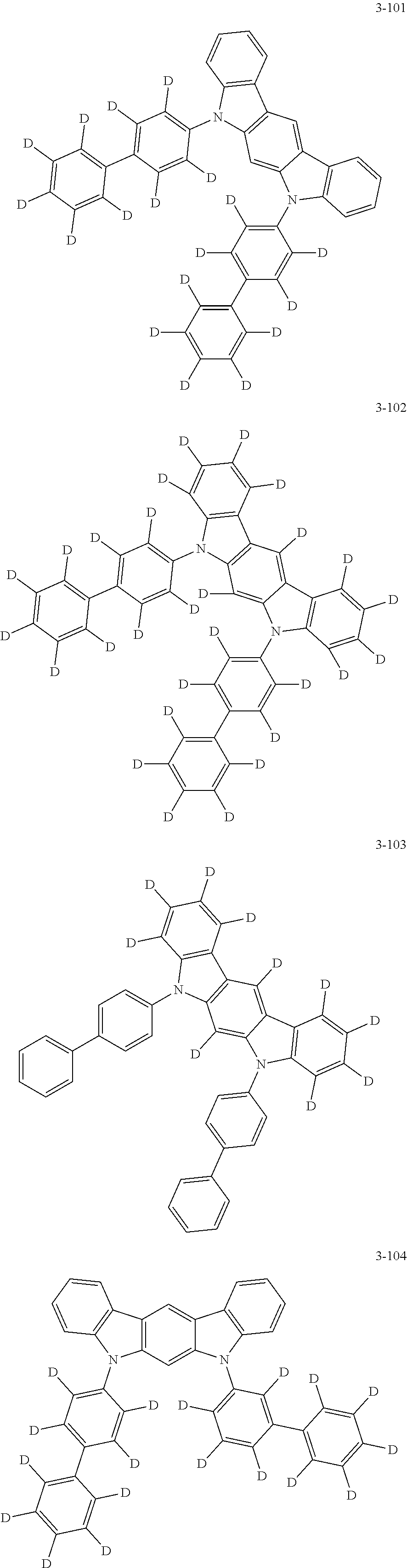

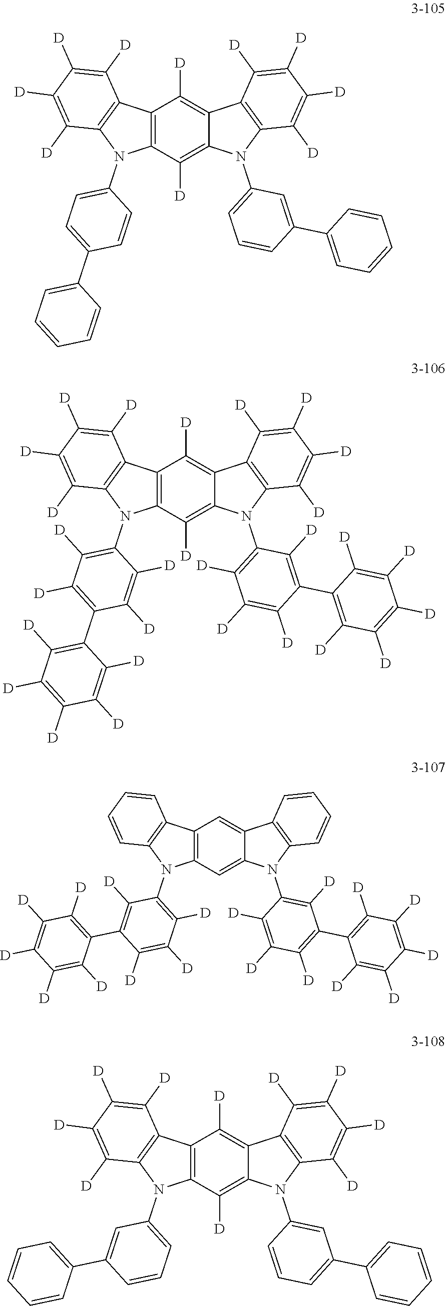

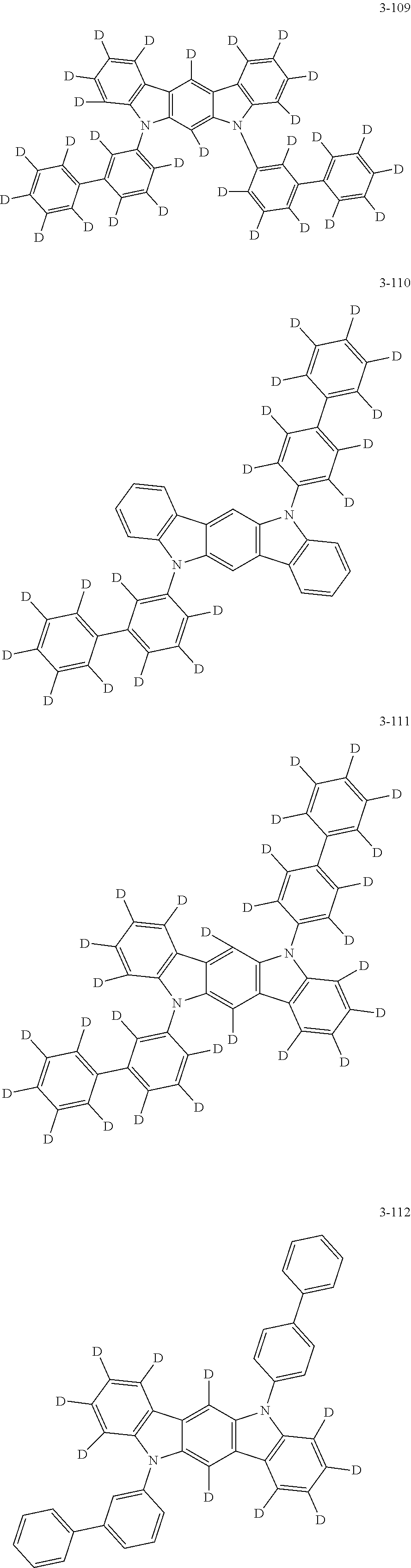

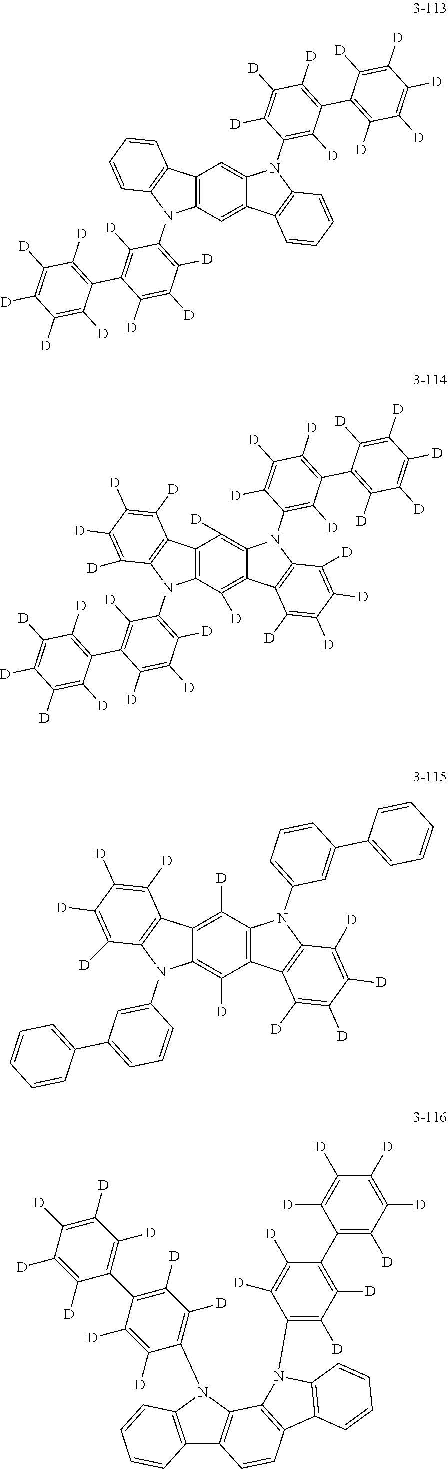

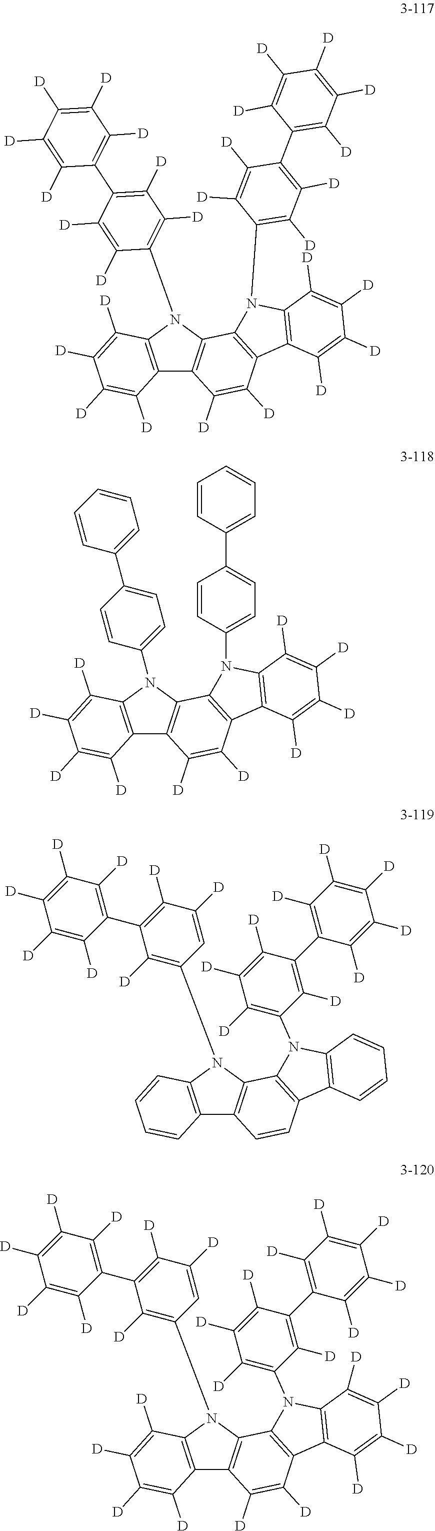

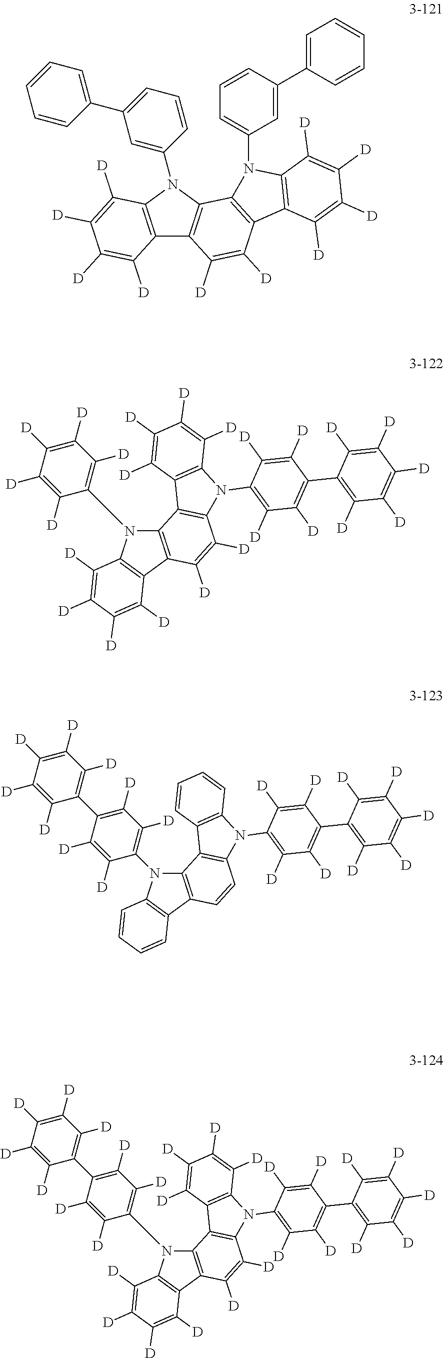

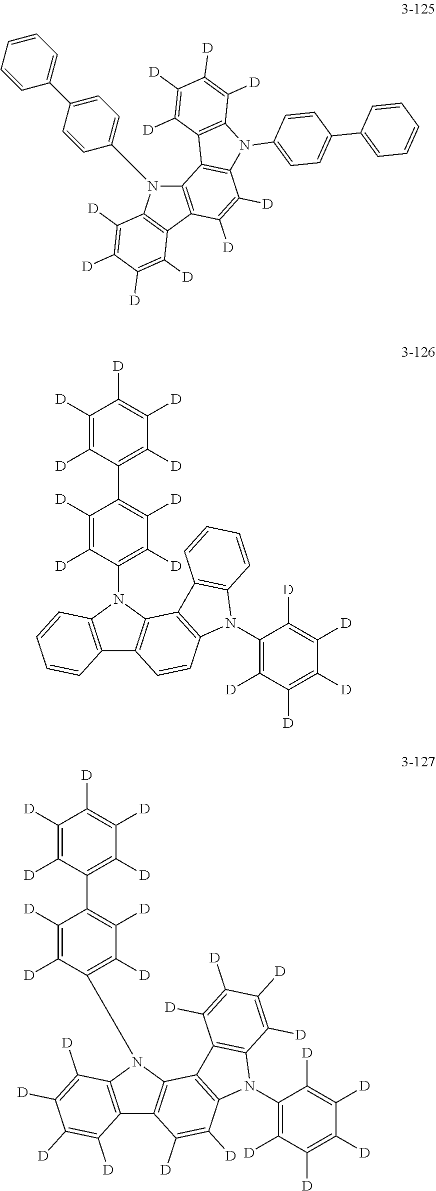 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccc(-c2ccccc2)cc1 ; [2H]c1c([2H])c([2H])c(-c2c([2H])c([2H])c(-n3c4ccccc4c4ccc5c(c6ccccc6n5-c5c([2H])c([2H])c([2H])c([2H])c5[2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)c([2H])c2[2H])c([2H])c1[2H]]

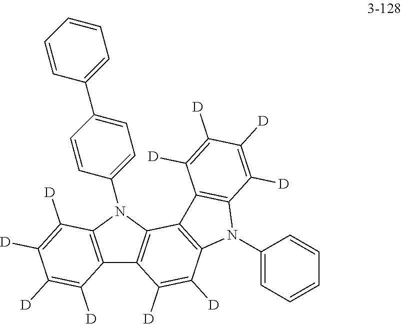 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccccc1]

As long as the unique characteristics of the heterocyclic compound represented by Chemical Formula 2 or 3 are maintained, various substituents may be introduced in addition to the structures exemplified above to synthesize a heterocyclic compound to which the characteristics of the introduced substituents are added. For example, it is possible to synthesize a material which satisfies the conditions required for each organic material layer by introducing into the core structure a substituent usually used for a hole injection layer material, a hole transport layer material, a hole transport auxiliary layer material, a light emitting layer material, an electron transport layer material, an electron transport auxiliary layer material, and an electron blocking layer material used during the manufacture of an organic light emitting device.

In addition, it is possible to finely adjust an energy band-gap by introducing various substituents into the heterocyclic compound structure represented by Chemical Formula 2 or 3, and meanwhile, it is possible to improve characteristics at the interface between organic materials and diversify the use of the material.

According to an exemplary embodiment of the present specification, the first electrode may be a positive electrode, and the second electrode may be a negative electrode.

According to an exemplary embodiment of the present specification, the first electrode may be a negative electrode, and the second electrode may be a positive electrode.

According to an exemplary embodiment of the present specification, the organic light emitting device may be a blue organic light emitting device, and the heterocyclic compound represented by Chemical Formula 1 may be used as a material for the blue organic light emitting device.

According to an exemplary embodiment of the present specification, the heterocyclic compound represented by Chemical Formula 1 and the heterocyclic compound represented by Chemical Formula 2 or 3 may be used as a material for the blue organic light emitting device.

According to an exemplary embodiment of the present specification, the organic light emitting device may be a green organic light emitting device, and the heterocyclic compound represented by Chemical Formula 1 may be used as a material for the green organic light emitting device.

According to an exemplary embodiment of the present specification, the heterocyclic compound represented by Chemical Formula 1 and the heterocyclic compound represented by Chemical Formula 2 or 3 may be used as a material for the green organic light emitting device.

According to an exemplary embodiment of the present specification, the organic light emitting device may be a red organic light emitting device, and the heterocyclic compound represented by Chemical Formula 1 may be used as a material for the red organic light emitting device.

According to an exemplary embodiment of the present specification, the heterocyclic compound represented by Chemical Formula 1 and the heterocyclic compound represented by Chemical Formula 2 or 3 may be used as a material for the red organic light emitting device.

The organic material layer of the organic light emitting device of the present specification may also have a single-layered structure, but may have a multi-layered structure in which two or more organic material layers are stacked. For example, the organic light emitting device of the present specification may have a structure including a hole injection layer, a hole transport layer, a light emitting layer, an electron transport layer, an electron injection layer, and the like as organic material layers. However, the structure of the organic light emitting device is not limited thereto, and may include less or more numbers of organic material layers.

According to an exemplary embodiment of the present specification, the organic material layer may include an iridium-based dopant.

According to an exemplary embodiment of the present specification, as the iridium-based dopant, Ir(ppy)₃, which is a green phosphorescent dopant, may be used, but the iridium-based dopant is not limited thereto.

According to an exemplary embodiment of the present specification, as the iridium-based dopant, (piq)₂(Ir)(acac), which is a red phosphorescent dopant, may be used, but the iridium-based dopant is not limited thereto.

In the organic light emitting device of the present specification, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

In the organic light emitting device of the present specification, as a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

In the organic light emitting device of the present specification, as a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

In the organic light emitting device of the present specification, as a hole transport material, a pyrazoline derivative, an arylamine-based derivative, a stilbene derivative, a triphenyldiamine derivative, and the like may be used, and a low-molecular weight or polymer material may also be used.

In the organic light emitting device of the present specification, as an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

In the organic light emitting device of the present specification, as an electron injection material, for example, LiF is representatively used in the art, but the present specification is not limited thereto.

In the organic light emitting device of the present specification, as a light emitting material, a red, green, or blue light emitting material may be further used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from N-type host materials or P-type host materials may be used as a host material for a light emitting layer.

According to an exemplary embodiment of the present specification, the heterocyclic compound represented by Chemical Formula 1 may be used as an N-type host material, and the heterocyclic compound represented by Chemical Formula 2 or 3 may be used as a P-type host material.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

The organic light emitting device of the present specification may further include one or two or more layers selected from the group consisting of a light emitting layer, a hole injection layer, a hole transport layer, a hole transport auxiliary layer, an electron injection layer, an electron transport layer, an electron blocking layer, and a hole blocking layer.

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

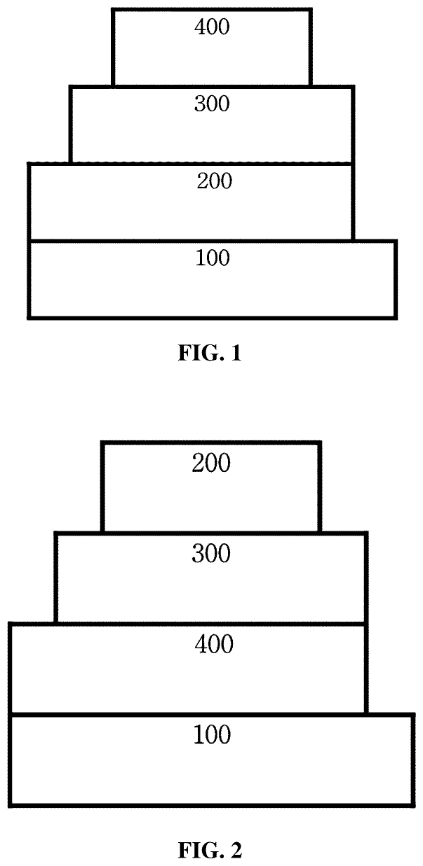

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented. The composition for an organic light emitting device may be included in the organic material layer 300, and the organic material layer 300 may be one or more layers.

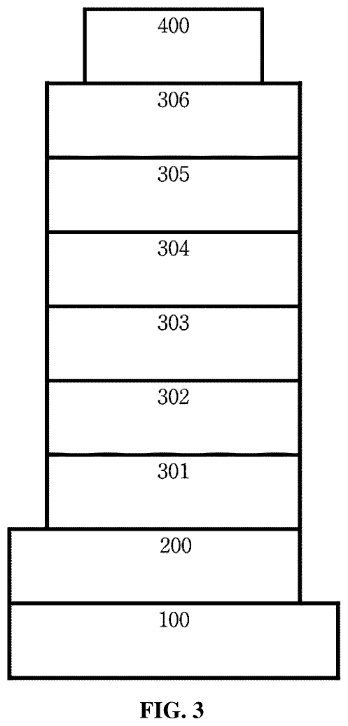

FIG. 3 exemplifies a case where a positive electrode 200 is provided on a substrate 100 and an organic material layer is provided between the positive electrode 200 and a negative electrode 400 in a multi-layer structure. The organic light emitting device according to FIG. 3 may include a hole injection layer 301, a hole transport layer 302, a light emitting layer 303, a hole blocking layer 304, an electron transport layer 305, and an electron injection layer 306 as an organic material layer, and may include the heterocyclic compound represented by Chemical Formula 1 in the light emitting layer 303. Alternatively, the organic light emitting device according to FIG. 3 may further include the heterocyclic compound represented by Chemical Formula 2 or 3 in the light emitting layer 303 including the heterocyclic compound represented by Chemical Formula 1.

The scope of the present application is not limited by the stacking structure as described above, and if necessary, the other layers except for the light emitting layer may be omitted, and another necessary functional layer may be further added.

The heterocyclic compound represented by Chemical Formula 1 may be used in forming an organic material layer of an organic light emitting device, and may be more preferably used particularly as a light emitting material.

If necessary, when different types of compounds other than the heterocyclic compound represented by the above-described Chemical Formula 1 and/or the heterocyclic compound represented by the above-described Chemical Formula 2 or 3 are mixed to form a mixture, the mixture may be in a premixed form, and a powder-state material may be mixed before forming the organic material layer of the organic light emitting device, and a compound that is in a liquid state at or above a suitable temperature may be mixed. The composition is in a solid state at a temperature which is equal to or less than the melting point of each material, and may be maintained as a liquid phase when the temperature is adjusted.

The heterocyclic compound represented by Chemical Formula 1 and the heterocyclic compound represented by Chemical Formula 2 or 3 may further include materials publicly-known in the art, such as solvents and additives.

### <Method for Manufacturing Organic Light Emitting Device>

According to an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, wherein the forming of the organic material layer includes forming an organic material layer having one or more layers using the heterocyclic compound (means the heterocyclic compound represented by Chemical Formula 1, and may further mean a composition when the heterocyclic compound represented by Chemical Formula 2 or 3 is added).

According to an exemplary embodiment of the present specification, in the forming of the organic material layer, the heterocyclic compound may be formed using a thermal vacuum deposition method.

The organic light emitting device according to an exemplary embodiment of the present specification may be manufactured by typical manufacturing methods and materials of the organic light emitting device, except that the heterocyclic compound is used to form an organic material layer.

Specifically, for a method of forming an organic material layer, an organic material layer may be formed by not only a vacuum deposition method, but also a solution application method when the organic light emitting device is manufactured. Herein, the solution coating method means spin coating, dip coating, inkjet printing, screen printing, a spray method, roll coating, and the like, but is not limited thereto.

### <Composition for Organic Material Layer of Organic Light Emitting Device>

According to an exemplary embodiment of the present specification, provided is a composition for forming an organic material layer of an organic light emitting device, including the above-described heterocyclic compound (represented by Chemical Formula 1) and a heterocyclic compound represented by the following Chemical Formula 2 or 3.

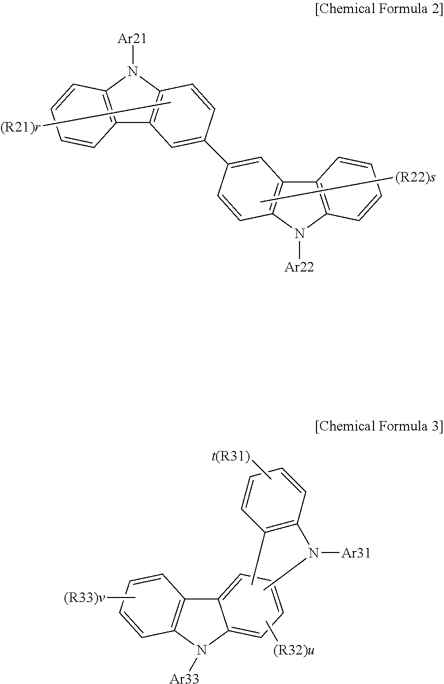

In Chemical Formulae 2 and 3, the description of each substituent is the same as that described above.

In an exemplary embodiment, the composition for an organic material layer may include the heterocyclic compound (represented by Chemical Formula 1) and the heterocyclic compound represented by Chemical Formula 2 or 3 at a weight ratio of 1:10 to 10:1.

In an exemplary embodiment, the composition for an organic material layer may include the heterocyclic compound (represented by Chemical Formula 1) and the heterocyclic compound represented by Chemical Formula 2 or 3 at a weight ratio of 1:8 to 8:1, 1:5 to 5:1, or 1:3 to 3:1.

According to the exemplary embodiments, when the heterocyclic compound represented by Chemical Formula 1 and the heterocyclic compound represented by Chemical Formula 2 or 3 are simultaneously included, an exciplex phenomenon occurs, so that the driving voltage of an organic light emitting device may be further lowered, and the light emitting efficiency and service life may be further improved when the composition for an organic material layer is used as a composition for an organic material layer of an organic light emitting device.

Hereinafter, the present specification will be described in more detail through Examples, but these Examples are provided only for exemplifying the present application, and are not intended to limit the scope of the present application.

### PREPARATION EXAMPLES

### [Preparation Example 1] Preparation of Compound 1, and the Like

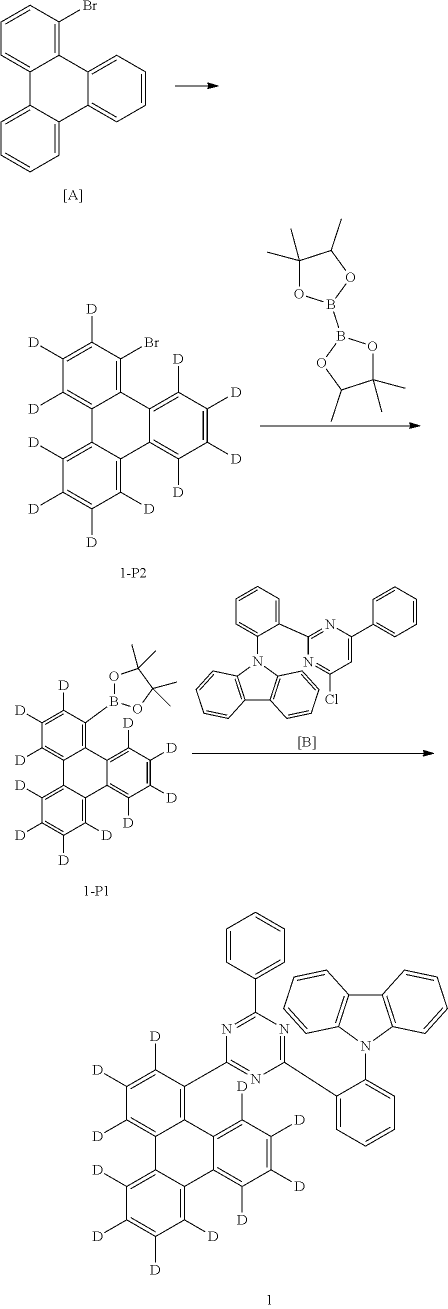

### 1) Preparation of Compound 1-P2

After 20 g (65.11 mmol, [A]) of 1-bromotriphenylene was dissolved in 600 mL (677.36 mmol) of benzene-d₆, 117 mL (1302.12 mmol) of triflic acid was added thereto, and the resulting mixture was stirred at 60° C. for 16 hours. After the reaction was completed, the reaction solution is neutralized by adding D₂O and Na₂CO₃, ethyl acetate was added thereto and dissolved, and then a product was extracted with distilled water. After the organic layer was dried over anhydrous MgSO₄, the solvent was removed by a rotary evaporator, and then the residue was purified by column chromatography using dichloromethane and hexane as eluting solvents, thereby obtaining 14.9 g (yield 72%) of Compound 1-P2.

### 2) Preparation of Compound 1-P1

After 14.9 g (46.88 mmol) of Compound 1-P2 and 10.59 g (46.88 mmol) of 4,4,4′,4′,5,5′-hexamethyl-2,2′-bi(1,3,2-dioxaborolane) were dissolved in 150 mL of 1,4-dioxane, 4.29 g (4.68 mmol) of tris(dibenzylideneacetone)dipalladium (0) (Pd₂(dba)₃), 13.80 g (140.64 mmol) of potassium acetate, and 4.47 g (9.37 mmol) of 2-dicyclohexylphosphino-2′,4′,6′-triisopropylbiphenyl (Xphos) were added thereto, and the resulting mixture was stirred under reflux for 16 hours.

After the reaction was completed, ethyl acetate was added to the reaction solution for dissolution, and then the resulting solution was extracted with distilled water, the organic layer was dried over anhydrous MgSO₄, and then the solvent was removed using a rotary evaporator, and then the residue was purified by column chromatography using dichloromethane and hexane as eluting solvents, thereby obtaining 12.0 g (yield 70%) of Compound 1-P1.

### 3) Preparation of Compound 1

After 12.0 g (32.96 mmol) of Compound 1-P1 and 14.27 g of (32.96 mmol, [B]) of 9-(2-(4-chloro-6-phenyl-1,3,5-triazin-2-yl)phenyl)-9H-carbazole were dissolve in 150 mL of 1,4-dioxane and 30 mL of distilled water, 3.81 g (3.29 mmol) of Pd(PPh₃)₄ and 13.66 g (98.87 mmol) of K₂CO₃ were added thereto, and the resulting mixture was stirred under reflux for 16 hours.

After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 12.24 g (yield 58%) of Compound 1.

The target compounds in the following Table 1 were synthesized in the same manner as above, except that Compound A′ in the following Table 1 was used instead of Compound [A], Compound B′ in the following Table 1 was used instead of Compound [B], and in the case of Compounds 503, 511, and 516, a smaller equivalent of triflic acid was used.

TABLE 1

| No. | Compound A′ | Compound B′ | Target Compound | Yield |
|---|---|---|---|---|
| 1 | 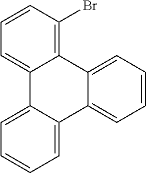 [SMILES: Brc1cccc2c3ccccc3c3ccccc3c12] | 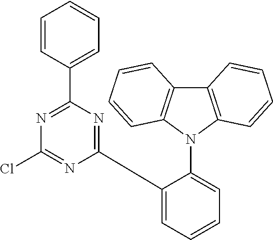 [SMILES: Clc1nc(-c2ccccc2)nc(-c2ccccc2-n2c3ccccc3c3ccccc32)n1] | 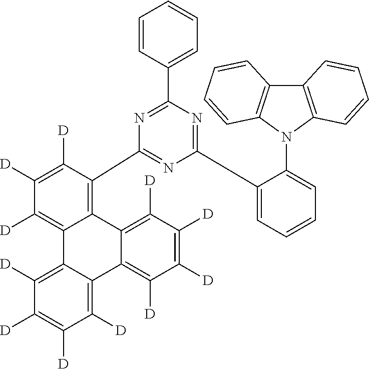 [SMILES: [2H]c1c([2H])c([2H])c2c(c1[2H])c1c([2H])c([2H])c([2H])c([2H])c1c1c(-c3nc(-c4ccccc4)nc(-c4ccccc4-n4c5ccccc5c5ccccc54)n3)c([2H])c([2H])c([2H])c21] | 58% |
| 4 | | 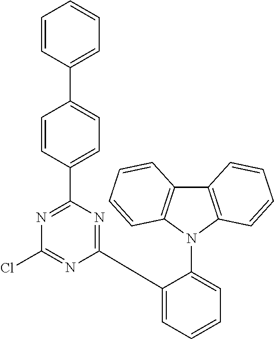 [SMILES: Clc1nc(-c2ccc(-c3ccccc3)cc2)nc(-c2ccccc2-n2c3ccccc3c3ccccc32)n1] | 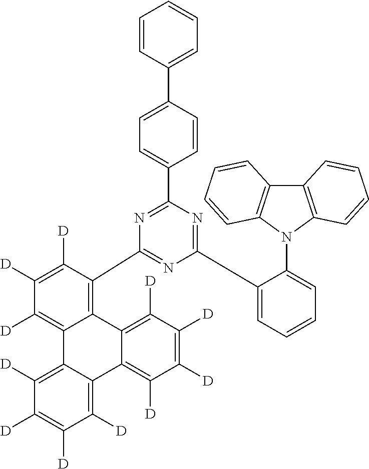 [SMILES: [2H]c1c([2H])c([2H])c2c(c1[2H])c1c([2H])c([2H])c([2H])c([2H])c1c1c(-c3nc(-c4ccc(-c5ccccc5)cc4)nc(-c4ccccc4-n4c5ccccc5c5ccccc54)n3)c([2H])c([2H])c([2H])c21] | 55% |
| 5 | | 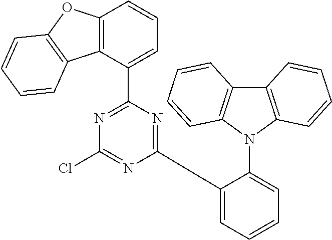 [SMILES: Clc1nc(-c2ccccc2-n2c3ccccc3c3ccccc32)nc(-c2cccc3oc4ccccc4c23)n1] | 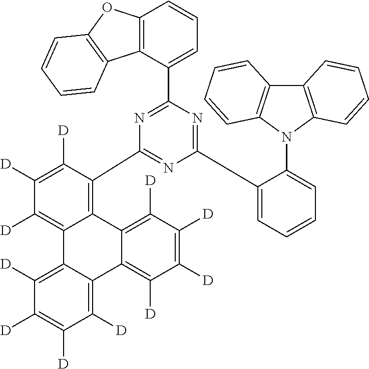 [SMILES: [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5oc6ccccc6c45)n3)c([2H])c([2H])c([2H])c21] | 54% |
| 7 | | 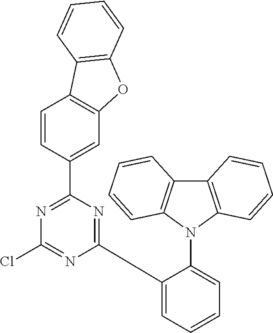 [SMILES: Clc1nc(-c2ccc3c(c2)oc2ccccc23)nc(-c2ccccc2-n2c3ccccc3c3ccccc32)n1] | 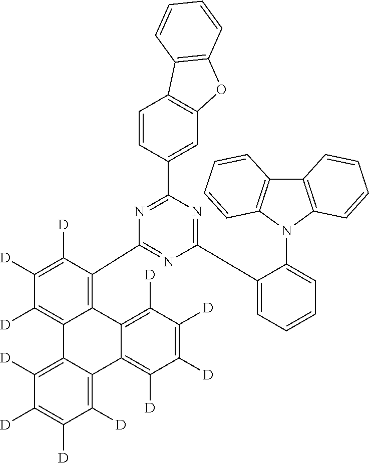 [SMILES: [2H]c1c([2H])c([2H])c2c(c1[2H])c1c([2H])c([2H])c([2H])c([2H])c1c1c(-c3nc(-c4ccc5c(c4)oc4ccccc45)nc(-c4ccccc4-n4c5ccccc5c5ccccc54)n3)c([2H])c([2H])c([2H])c21] | 60% |
| 10 | | 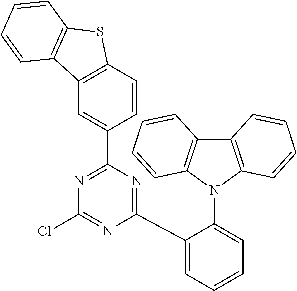 [SMILES: Clc1nc(-c2ccc3sc4ccccc4c3c2)nc(-c2ccccc2-n2c3ccccc3c3ccccc32)n1] | 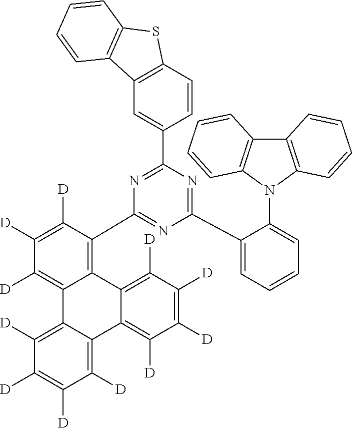 [SMILES: [2H]c1c([2H])c([2H])c2c(c1[2H])c1c([2H])c([2H])c([2H])c([2H])c1c1c(-c3nc(-c4ccc5sc6ccccc6c5c4)nc(-c4ccccc4-n4c5ccccc5c5ccccc54)n3)c([2H])c([2H])c([2H])c21] | 61% |
| 12 | 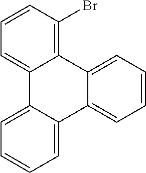 [SMILES: Brc1cccc2c3ccccc3c3ccccc3c12] | 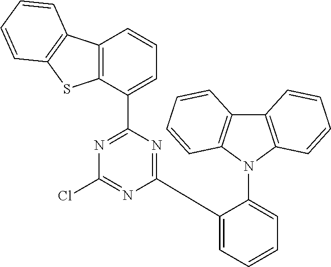 [SMILES: Clc1nc(-c2ccccc2-n2c3ccccc3c3ccccc32)nc(-c2cccc3c2sc2ccccc23)n1] | 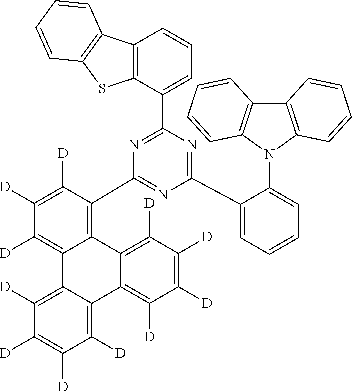 [SMILES: [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5c4sc4ccccc45)n3)c([2H])c([2H])c([2H])c21] | 52% |
| 14 | | 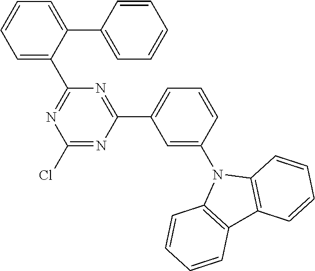 [SMILES: Clc1nc(-c2cccc(-n3c4ccccc4c4ccccc43)c2)nc(-c2ccccc2-c2ccccc2)n1] | 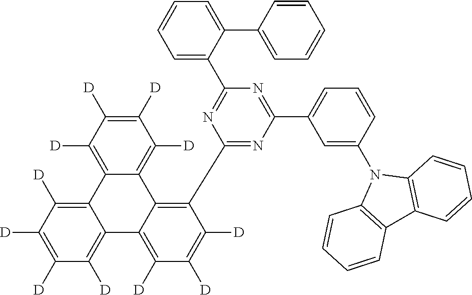 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccccc4-c4ccccc4)n3)c([2H])c([2H])c([2H])c21] | 51% |
| 15 | | 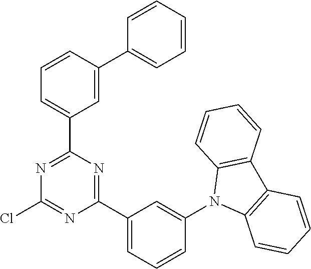 [SMILES: Clc1nc(-c2cccc(-c3ccccc3)c2)nc(-c2cccc(-n3c4ccccc4c4ccccc43)c2)n1] | 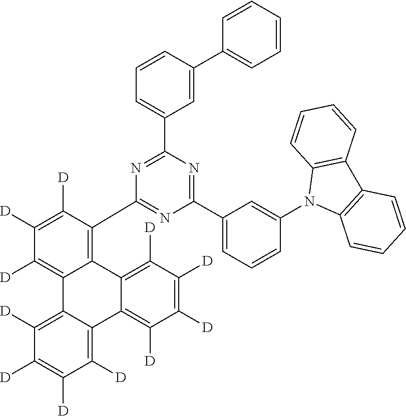 [SMILES: [2H]c1c([2H])c([2H])c2c(c1[2H])c1c([2H])c([2H])c([2H])c([2H])c1c1c(-c3nc(-c4cccc(-c5ccccc5)c4)nc(-c4cccc(-n5c6ccccc6c6ccccc65)c4)n3)c([2H])c([2H])c([2H])c21] | 52% |
| 18 | | 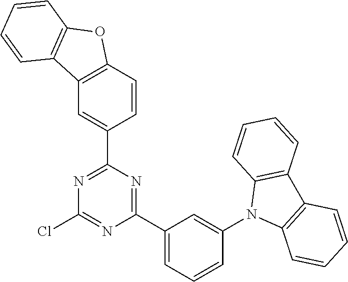 [SMILES: Clc1nc(-c2cccc(-n3c4ccccc4c4ccccc43)c2)nc(-c2ccc3oc4ccccc4c3c2)n1] | 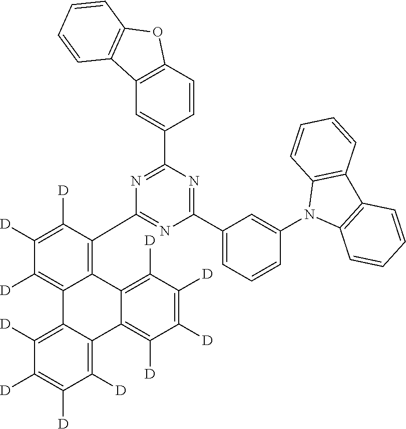 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5oc6ccccc6c5c4)n3)c([2H])c([2H])c([2H])c21] | 55% |
| 20 | | 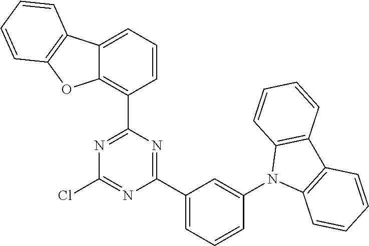 [SMILES: Clc1nc(-c2cccc(-n3c4ccccc4c4ccccc43)c2)nc(-c2cccc3c2oc2ccccc23)n1] | 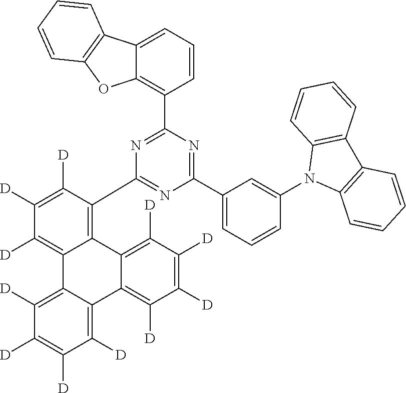 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5c4oc4ccccc45)n3)c([2H])c([2H])c([2H])c21] | 55% |
| 21 | | 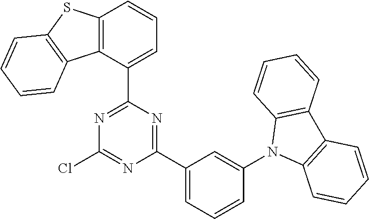 [SMILES: Clc1nc(-c2cccc(-n3c4ccccc4c4ccccc43)c2)nc(-c2cccc3sc4ccccc4c23)n1] | 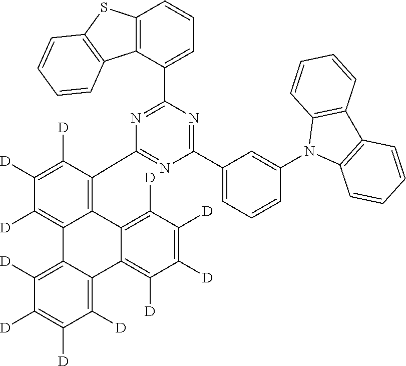 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5sc6ccccc6c45)n3)c([2H])c([2H])c([2H])c21] | 53% |
| 23 | 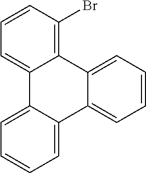 [SMILES: Brc1cccc2c3ccccc3c3ccccc3c12] | 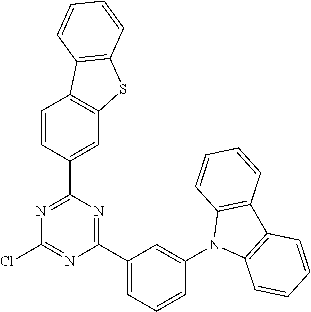 [SMILES: Clc1nc(-c2cccc(-n3c4ccccc4c4ccccc43)c2)nc(-c2ccc3c(c2)sc2ccccc23)n1] | 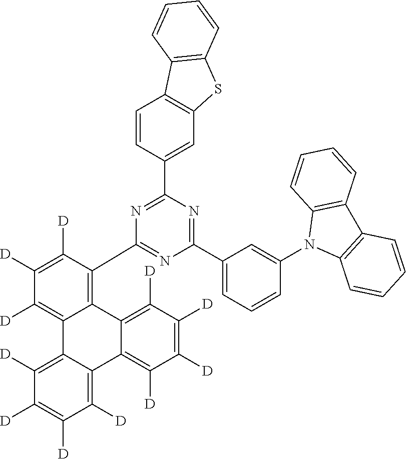 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5c(c4)sc4ccccc45)n3)c([2H])c([2H])c([2H])c21] | 65% |
| 25 | | 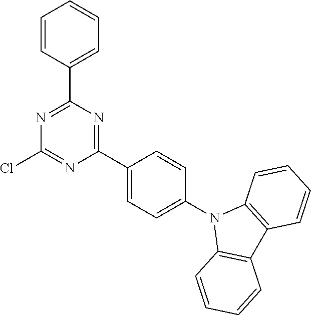 [SMILES: Clc1nc(-c2ccccc2)nc(-c2ccc(-n3c4ccccc4c4ccccc43)cc2)n1] | 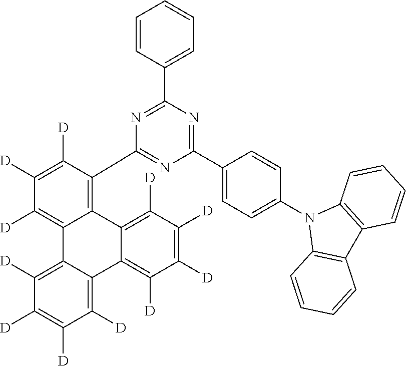 [SMILES: [2H]c1c([2H])c([2H])c2c(c1[2H])c1c([2H])c([2H])c([2H])c([2H])c1c1c(-c3nc(-c4ccccc4)nc(-c4ccc(-n5c6ccccc6c6ccccc65)cc4)n3)c([2H])c([2H])c([2H])c21] | 53% |
| 29 | | 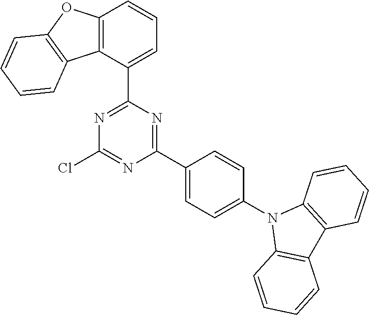 [SMILES: Clc1nc(-c2ccc(-n3c4ccccc4c4ccccc43)cc2)nc(-c2cccc3oc4ccccc4c23)n1] | 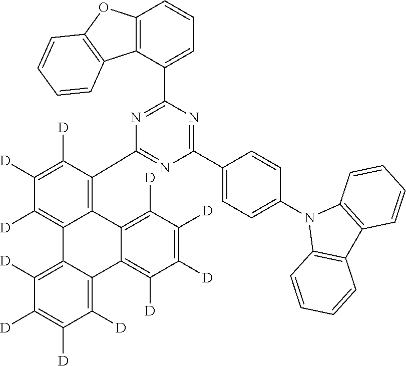 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5oc6ccccc6c45)n3)c([2H])c([2H])c([2H])c21] | 69% |
| 37 | 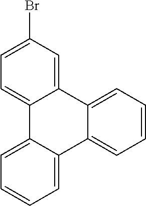 [SMILES: Brc1ccc2c3ccccc3c3ccccc3c2c1] | 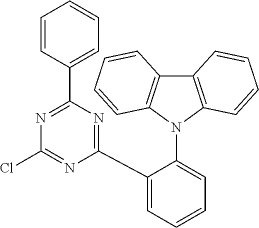 [SMILES: Clc1nc(-c2ccccc2)nc(-c2ccccc2-n2c3ccccc3c3ccccc32)n1] | 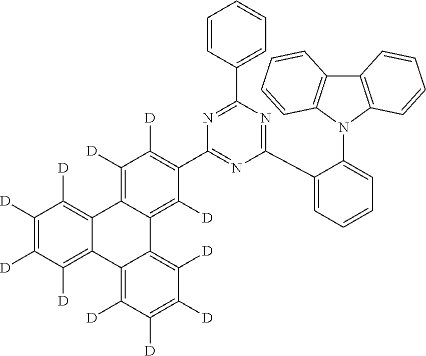 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccccc54)n3)c([2H])c([2H])c21] | 65% |
| 39 | | 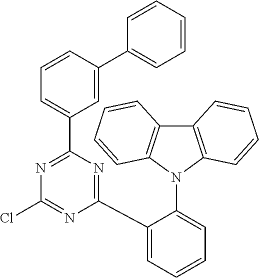 [SMILES: Clc1nc(-c2cccc(-c3ccccc3)c2)nc(-c2ccccc2-n2c3ccccc3c3ccccc32)n1] | 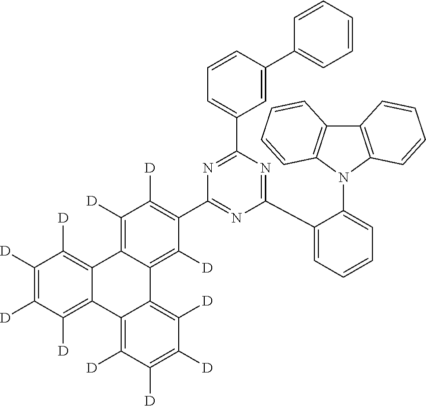 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-n4c5ccccc5c5ccccc54)n3)c([2H])c([2H])c21] | 58% |
| 41 | | 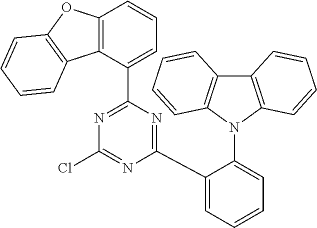 [SMILES: Clc1nc(-c2ccccc2-n2c3ccccc3c3ccccc32)nc(-c2cccc3oc4ccccc4c23)n1] | 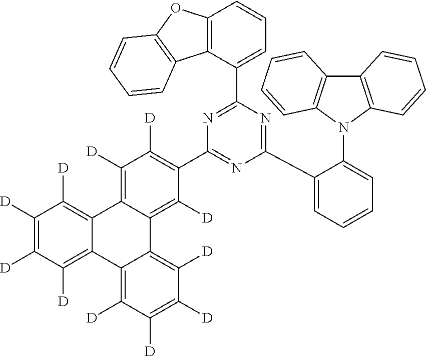 [SMILES: [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5oc6ccccc6c45)n3)c([2H])c([2H])c21] | 61% |
| 42 | | 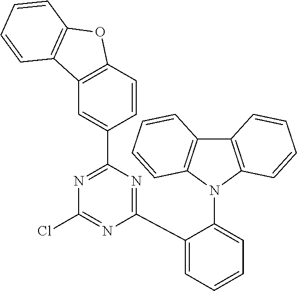 [SMILES: Clc1nc(-c2ccc3oc4ccccc4c3c2)nc(-c2ccccc2-n2c3ccccc3c3ccccc32)n1] | 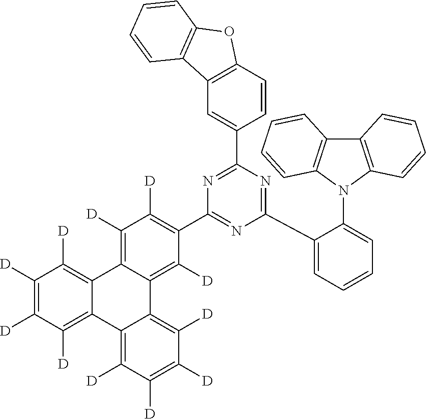 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-n4c5ccccc5c5ccccc54)n3)c([2H])c([2H])c21] | 54% |
| 43 | 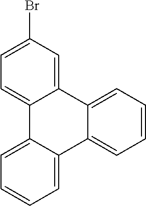 [SMILES: Brc1ccc2c3ccccc3c3ccccc3c2c1] | 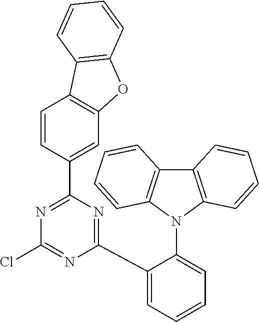 [SMILES: Clc1nc(-c2ccc3c(c2)oc2ccccc23)nc(-c2ccccc2-n2c3ccccc3c3ccccc32)n1] | 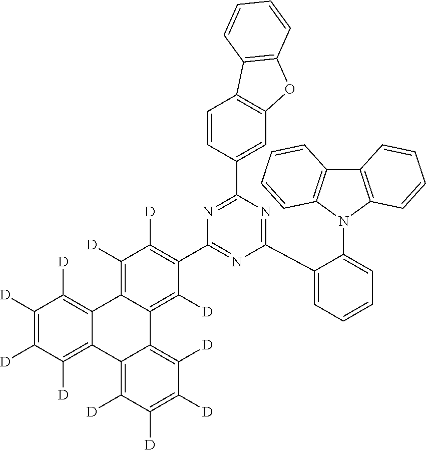 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-n4c5ccccc5c5ccccc54)n3)c([2H])c([2H])c21] | 66% |
| 44 | | 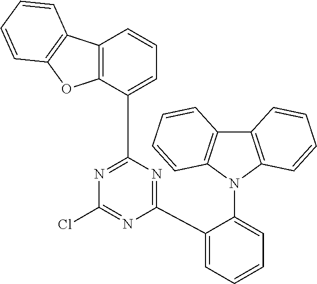 [SMILES: Clc1nc(-c2ccccc2-n2c3ccccc3c3ccccc32)nc(-c2cccc3c2oc2ccccc23)n1] | 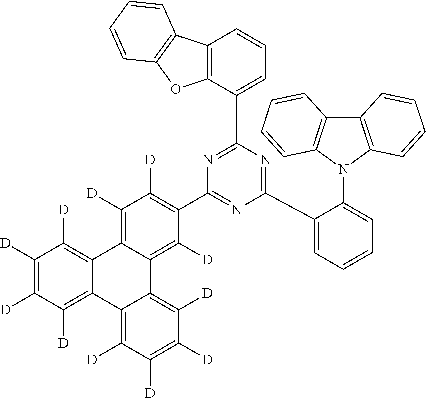 [SMILES: [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5c4oc4ccccc45)n3)c([2H])c([2H])c21] | 56% |
| 46 | | 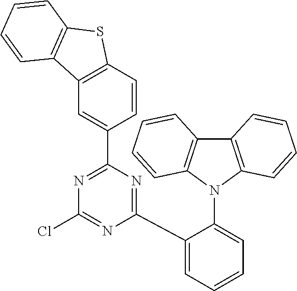 [SMILES: Clc1nc(-c2ccc3sc4ccccc4c3c2)nc(-c2ccccc2-n2c3ccccc3c3ccccc32)n1] | 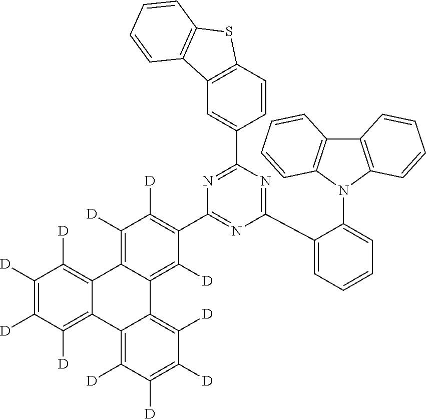 [SMILES: [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-n4c5ccccc5c5ccccc54)n3)c([2H])c([2H])c21] | 61% |
| 49 | | 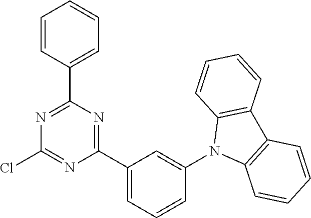 [SMILES: Clc1nc(-c2ccccc2)nc(-c2cccc(-n3c4ccccc4c4ccccc43)c2)n1] | 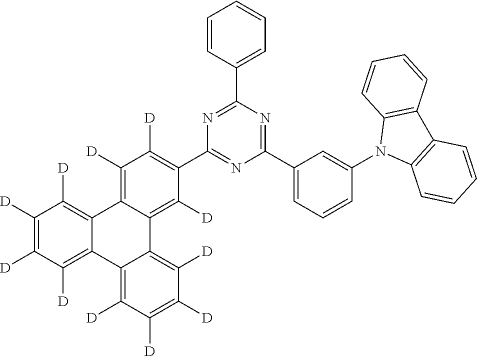 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccccc65)c4)n3)c([2H])c([2H])c21] | 64% |
| 50 | | 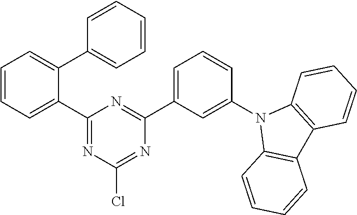 [SMILES: Clc1nc(-c2cccc(-n3c4ccccc4c4ccccc43)c2)nc(-c2ccccc2-c2ccccc2)n1] | 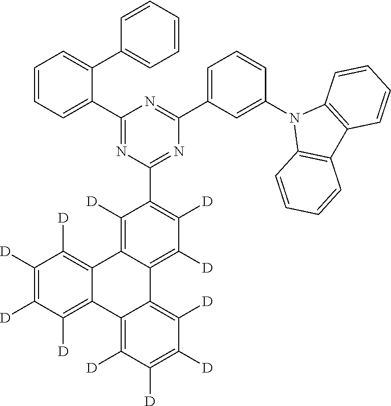 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccccc4-c4ccccc4)n3)c([2H])c([2H])c21] | 68% |
| 54 | | 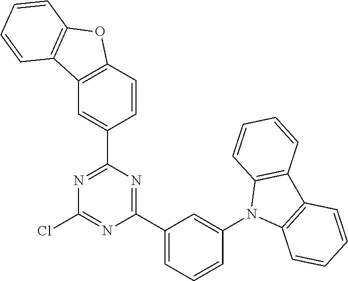 [SMILES: Clc1nc(-c2cccc(-n3c4ccccc4c4ccccc43)c2)nc(-c2ccc3oc4ccccc4c3c2)n1] | 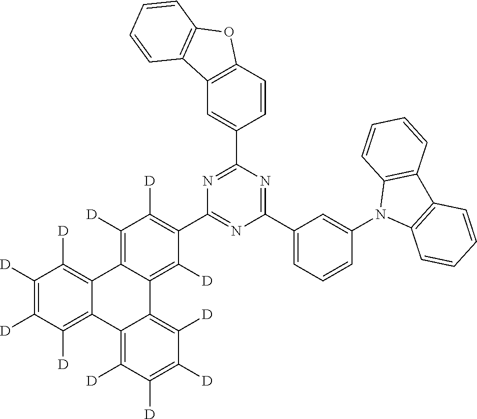 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5oc6ccccc6c5c4)n3)c([2H])c([2H])c21] | 61% |
| 56 | | 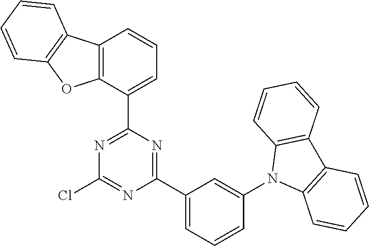 [SMILES: Clc1nc(-c2cccc(-n3c4ccccc4c4ccccc43)c2)nc(-c2cccc3c2oc2ccccc23)n1] | 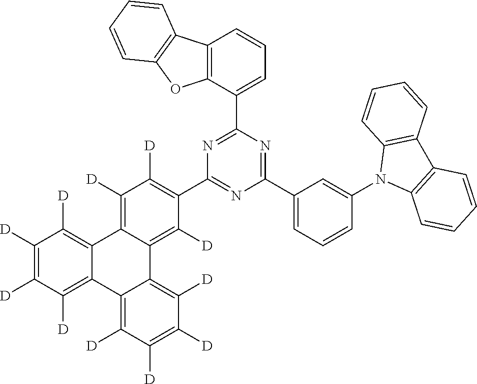 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5c4oc4ccccc45)n3)c([2H])c([2H])c21] | 66% |
| 61 | 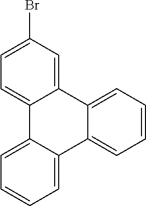 [SMILES: Brc1ccc2c3ccccc3c3ccccc3c2c1] | 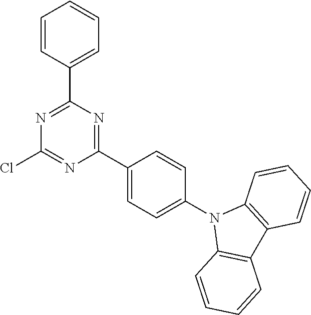 [SMILES: Clc1nc(-c2ccccc2)nc(-c2ccc(-n3c4ccccc4c4ccccc43)cc2)n1] | 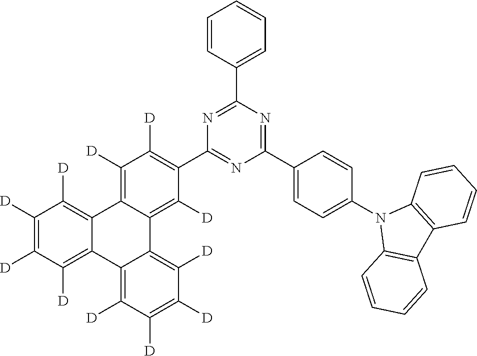 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-n5c6ccccc6c6ccccc65)cc4)n3)c([2H])c([2H])c21] | 60% |
| 63 | | 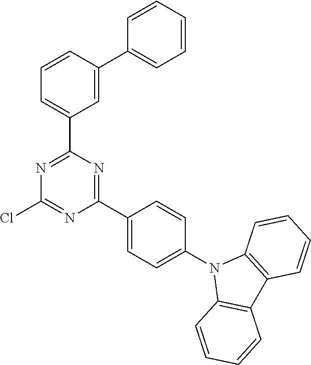 [SMILES: Clc1nc(-c2ccc(-n3c4ccccc4c4ccccc43)cc2)nc(-c2cccc(-c3ccccc3)c2)n1] | 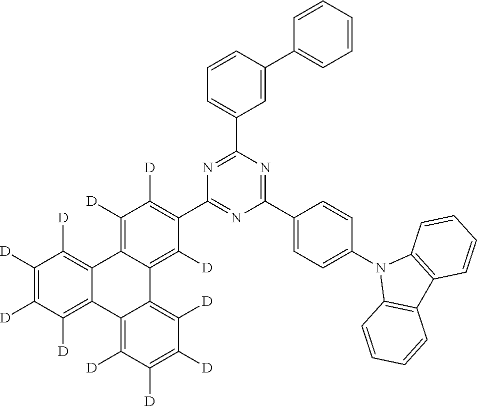 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc(-c5ccccc5)c4)n3)c([2H])c([2H])c21] | 67% |
| 68 | | 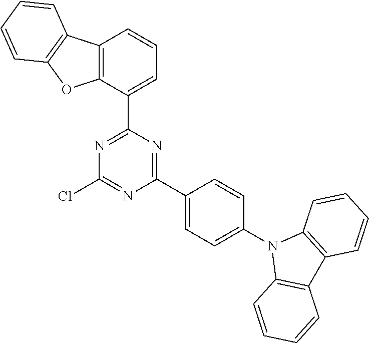 [SMILES: Clc1nc(-c2ccc(-n3c4ccccc4c4ccccc43)cc2)nc(-c2cccc3c2oc2ccccc23)n1] | 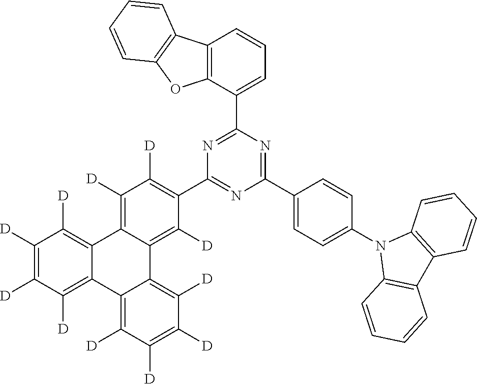 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5c4oc4ccccc45)n3)c([2H])c([2H])c21] | 63% |
| 70 | | 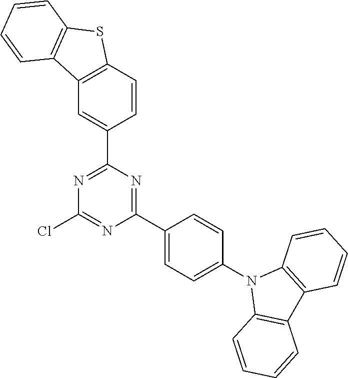 [SMILES: Clc1nc(-c2ccc(-n3c4ccccc4c4ccccc43)cc2)nc(-c2ccc3sc4ccccc4c3c2)n1] | 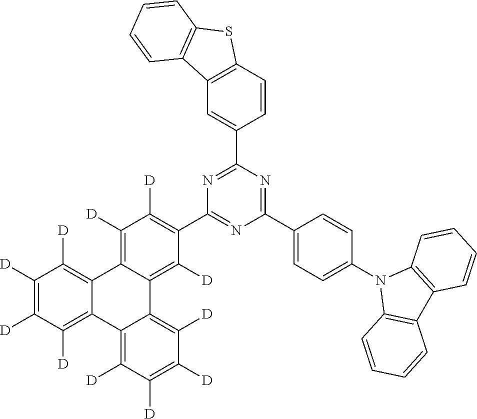 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5sc6ccccc6c5c4)n3)c([2H])c([2H])c21] | 61% |
| 73 | | 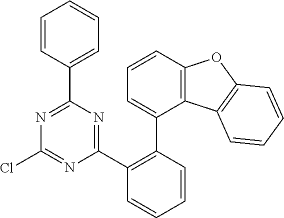 [SMILES: Clc1nc(-c2ccccc2)nc(-c2ccccc2-c2cccc3oc4ccccc4c23)n1] | 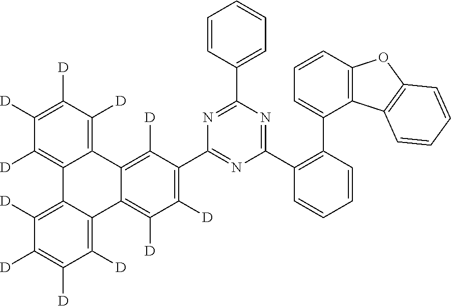 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5oc6ccccc6c45)n3)c([2H])c([2H])c21] | 54% |
| 76 | | 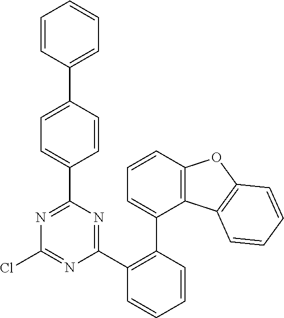 [SMILES: Clc1nc(-c2ccc(-c3ccccc3)cc2)nc(-c2ccccc2-c2cccc3oc4ccccc4c23)n1] | 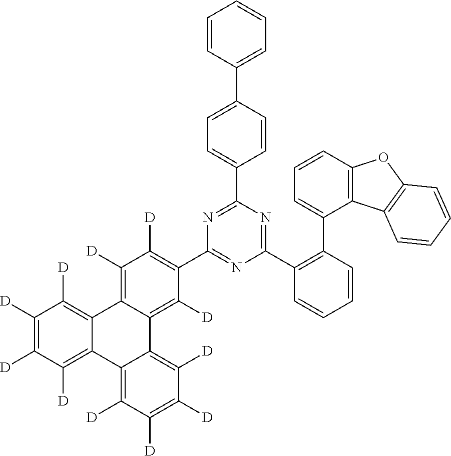 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5oc6ccccc6c45)n3)c([2H])c([2H])c21] | 54% |
| 77 | | 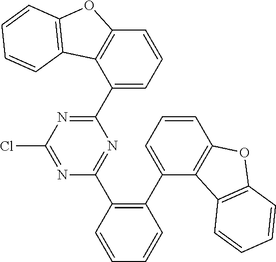 [SMILES: Clc1nc(-c2ccccc2-c2cccc3oc4ccccc4c23)nc(-c2cccc3oc4ccccc4c23)n1] | 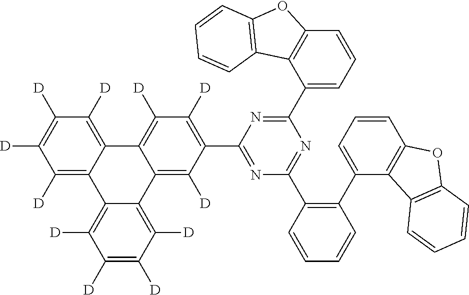 [SMILES: [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5oc6ccccc6c45)n3)c([2H])c([2H])c21] | 58% |
| 84 | 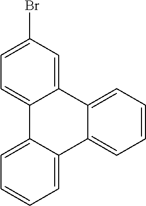 [SMILES: Brc1ccc2c3ccccc3c3ccccc3c2c1] | 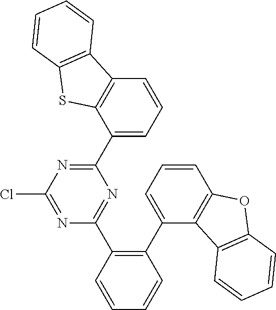 [SMILES: Clc1nc(-c2ccccc2-c2cccc3oc4ccccc4c23)nc(-c2cccc3c2sc2ccccc23)n1] | 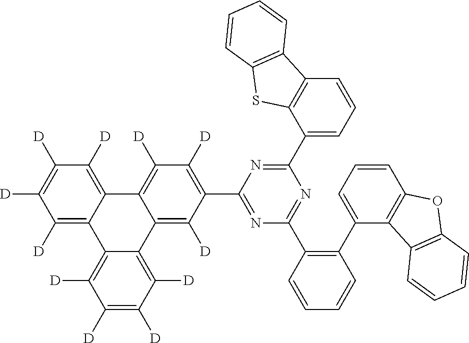 [SMILES: [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5c4sc4ccccc45)n3)c([2H])c([2H])c21] | 53% |
| 85 | | 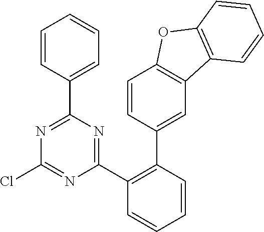 [SMILES: Clc1nc(-c2ccccc2)nc(-c2ccccc2-c2ccc3oc4ccccc4c3c2)n1] | 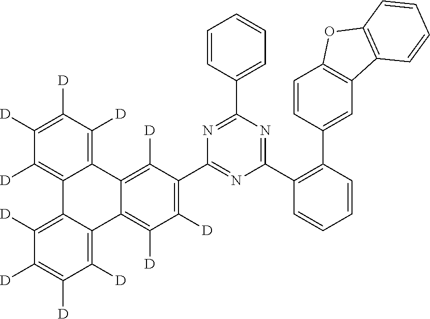 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5oc6ccccc6c5c4)n3)c([2H])c([2H])c21] | 52% |
| 86 | | 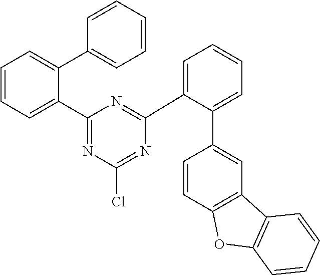 [SMILES: Clc1nc(-c2ccccc2-c2ccccc2)nc(-c2ccccc2-c2ccc3oc4ccccc4c3c2)n1] | 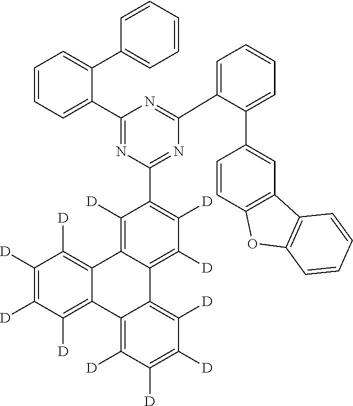 [SMILES: [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5oc6ccccc6c5c4)n3)c([2H])c([2H])c21] | 54% |
| 93 | | 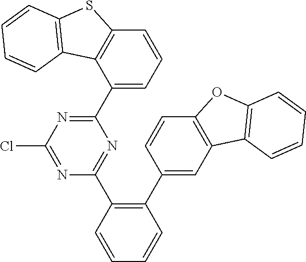 [SMILES: Clc1nc(-c2ccccc2-c2ccc3oc4ccccc4c3c2)nc(-c2cccc3sc4ccccc4c23)n1] | 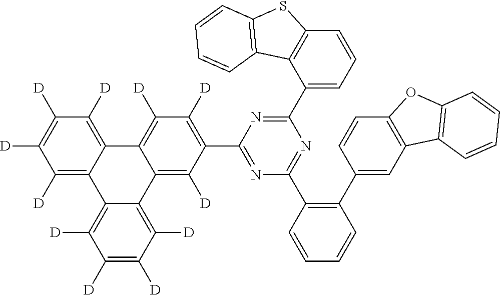 [SMILES: [2H]c1c([2H])c([2H])c2c(c1[2H])c1c([2H])c([2H])c([2H])c([2H])c1c1c([2H])c(-c3nc(-c4ccccc4-c4ccc5oc6ccccc6c5c4)nc(-c4cccc5sc6ccccc6c45)n3)c([2H])c([2H])c21] | 65% |
| 98 | | 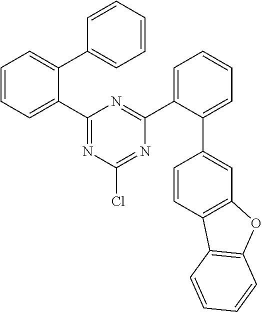 [SMILES: Clc1nc(-c2ccccc2-c2ccccc2)nc(-c2ccccc2-c2ccc3c(c2)oc2ccccc23)n1] | 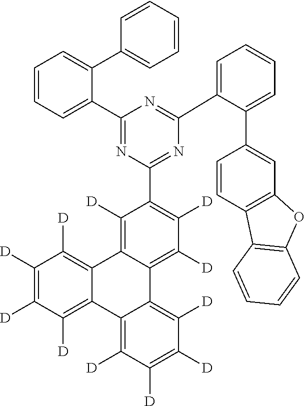 [SMILES: [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c(c4)oc4ccccc45)n3)c([2H])c([2H])c21] | 55% |
| 104 | | 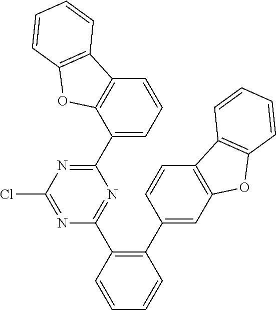 [SMILES: Clc1nc(-c2ccccc2-c2ccc3c(c2)oc2ccccc23)nc(-c2cccc3c2oc2ccccc23)n1] | 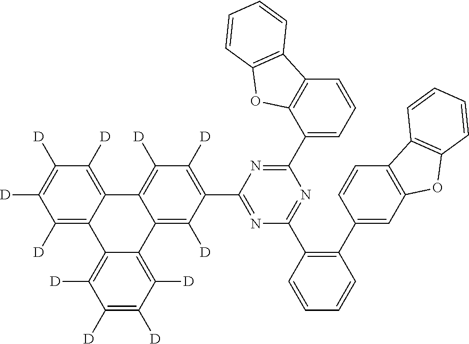 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5c4oc4ccccc45)n3)c([2H])c([2H])c21] | 60% |
| 109 | | 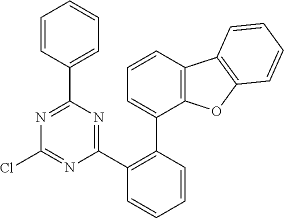 [SMILES: Clc1nc(-c2ccccc2)nc(-c2ccccc2-c2cccc3c2oc2ccccc23)n1] | 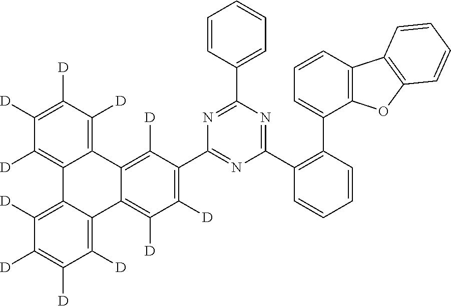 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4oc4ccccc45)n3)c([2H])c([2H])c21] | 58% |
| 111 | 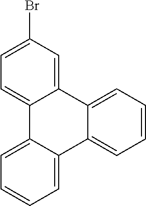 [SMILES: Brc1ccc2c3ccccc3c3ccccc3c2c1] | 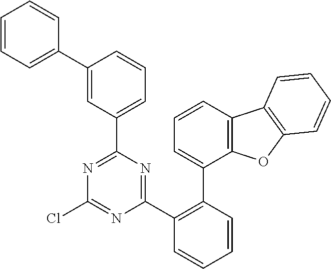 [SMILES: Clc1nc(-c2cccc(-c3ccccc3)c2)nc(-c2ccccc2-c2cccc3c2oc2ccccc23)n1] | 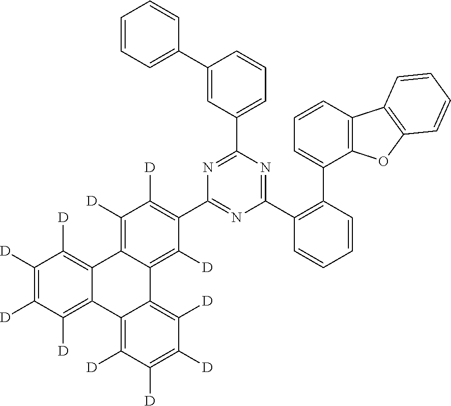 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c4oc4ccccc45)n3)c([2H])c([2H])c21] | 67% |
| 112 | | 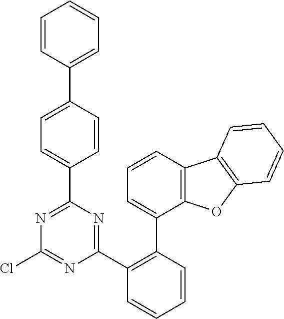 [SMILES: Clc1nc(-c2ccc(-c3ccccc3)cc2)nc(-c2ccccc2-c2cccc3c2oc2ccccc23)n1] | 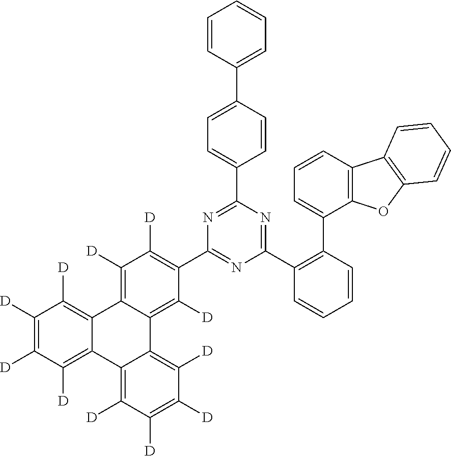 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c4oc4ccccc45)n3)c([2H])c([2H])c21] | 54% |
| 115 | | 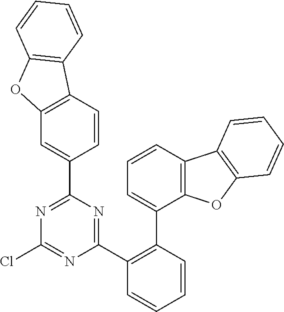 [SMILES: Clc1nc(-c2ccc3c(c2)oc2ccccc23)nc(-c2ccccc2-c2cccc3c2oc2ccccc23)n1] | 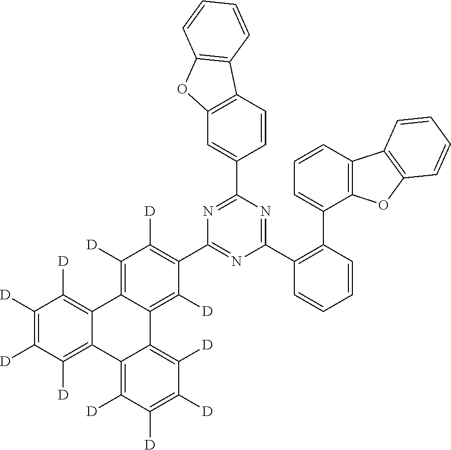 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c4oc4ccccc45)n3)c([2H])c([2H])c21] | 58% |
| 116 | | 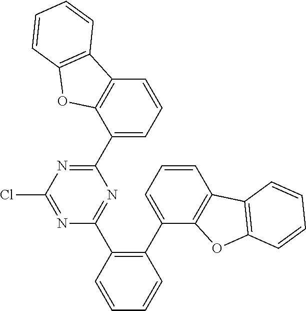 [SMILES: Clc1nc(-c2ccccc2-c2cccc3c2oc2ccccc23)nc(-c2cccc3c2oc2ccccc23)n1] | 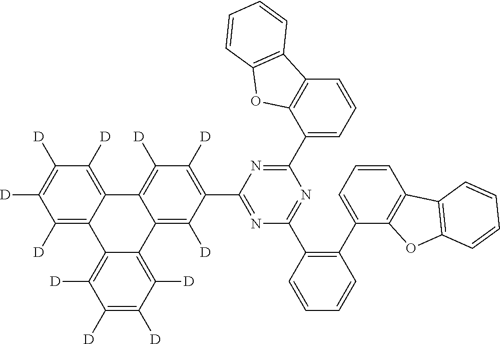 [SMILES: [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5c4oc4ccccc45)n3)c([2H])c([2H])c21] | 54% |
| 118 | | 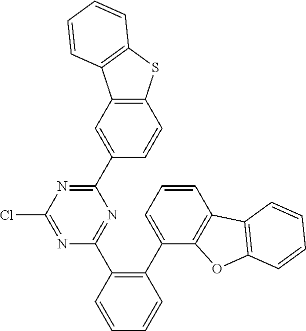 [SMILES: Clc1nc(-c2ccc3sc4ccccc4c3c2)nc(-c2ccccc2-c2cccc3c2oc2ccccc23)n1] | 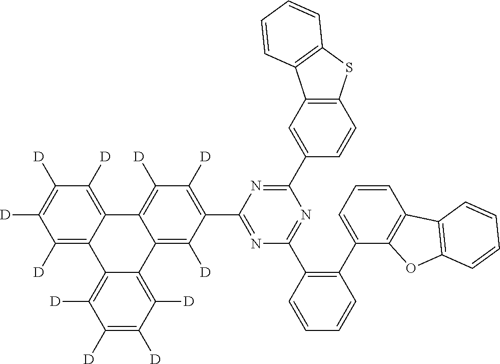 [SMILES: [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5c4oc4ccccc45)n3)c([2H])c([2H])c21] | 61% |
| 121 | | 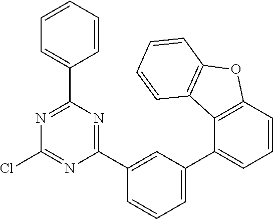 [SMILES: Clc1nc(-c2ccccc2)nc(-c2cccc(-c3cccc4oc5ccccc5c34)c2)n1] | 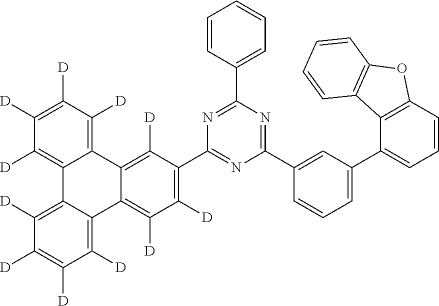 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6oc7ccccc7c56)c4)n3)c([2H])c([2H])c21] | 69% |
| 128 | | 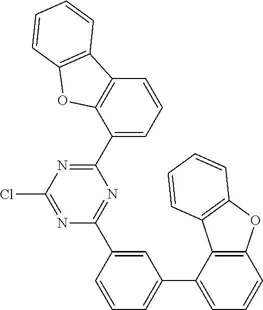 [SMILES: Clc1nc(-c2cccc(-c3cccc4oc5ccccc5c34)c2)nc(-c2cccc3c2oc2ccccc23)n1] | 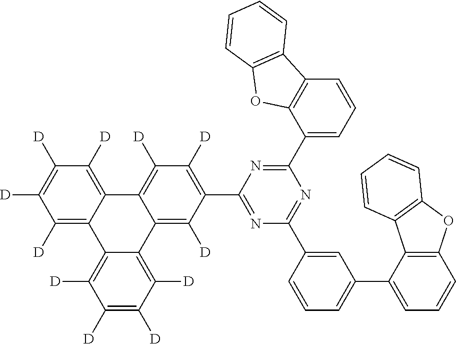 [SMILES: [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4cccc5c4oc4ccccc45)n3)c([2H])c([2H])c21] | 65% |
| 130 | 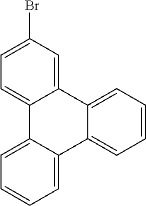 [SMILES: Brc1ccc2c3ccccc3c3ccccc3c2c1] | 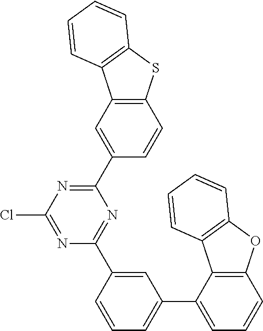 [SMILES: Clc1nc(-c2cccc(-c3cccc4oc5ccccc5c34)c2)nc(-c2ccc3sc4ccccc4c3c2)n1] | 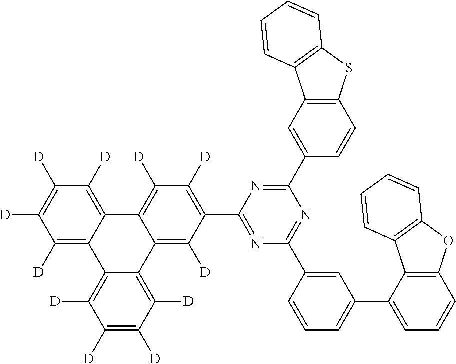 [SMILES: [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4ccc5sc6ccccc6c5c4)n3)c([2H])c([2H])c21] | 63% |
| 133 | | 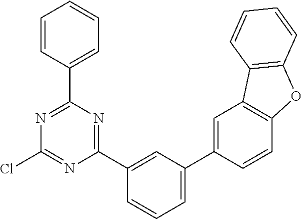 [SMILES: Clc1nc(-c2ccccc2)nc(-c2cccc(-c3ccc4oc5ccccc5c4c3)c2)n1] | 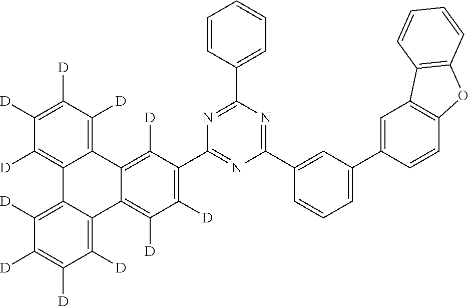 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6oc7ccccc7c6c5)c4)n3)c([2H])c([2H])c21] | 63% |
| 137 | | 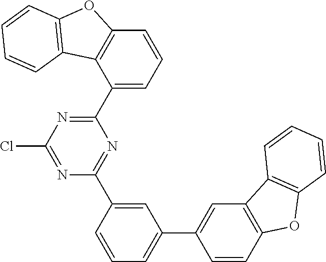 [SMILES: Clc1nc(-c2cccc(-c3ccc4oc5ccccc5c4c3)c2)nc(-c2cccc3oc4ccccc4c23)n1] | 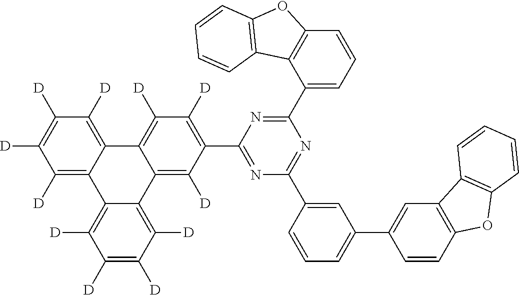 [SMILES: [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4cccc5oc6ccccc6c45)n3)c([2H])c([2H])c21] | 69% |
| 144 | | 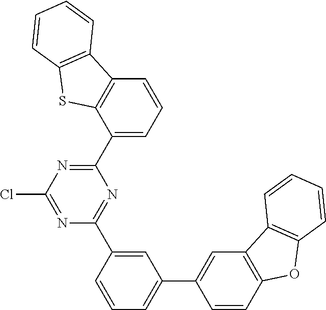 [SMILES: Clc1nc(-c2cccc(-c3ccc4oc5ccccc5c4c3)c2)nc(-c2cccc3c2sc2ccccc23)n1] | 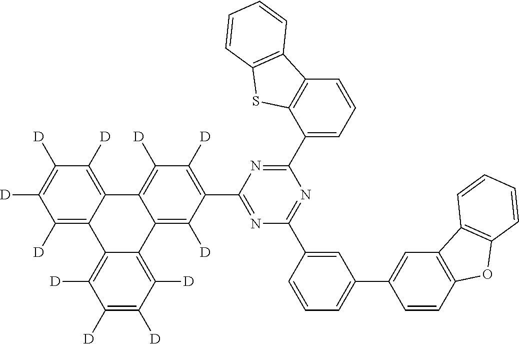 [SMILES: [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4cccc5c4sc4ccccc45)n3)c([2H])c([2H])c21] | 51% |
| 149 | | 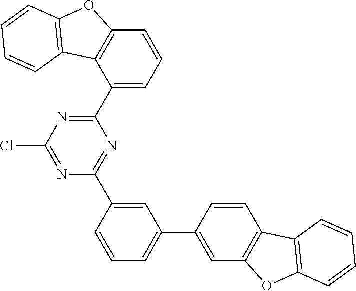 [SMILES: Clc1nc(-c2cccc(-c3ccc4c(c3)oc3ccccc34)c2)nc(-c2cccc3oc4ccccc4c23)n1] | 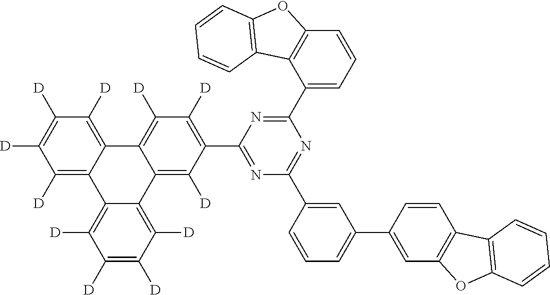 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4cccc5oc6ccccc6c45)n3)c([2H])c([2H])c21] | 63% |
| 156 | | 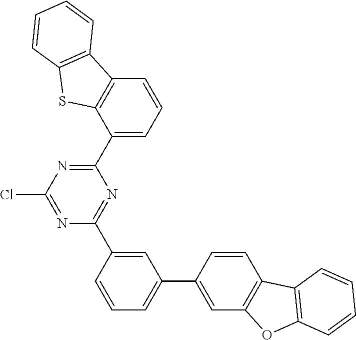 [SMILES: Clc1nc(-c2cccc(-c3ccc4c(c3)oc3ccccc34)c2)nc(-c2cccc3c2sc2ccccc23)n1] | 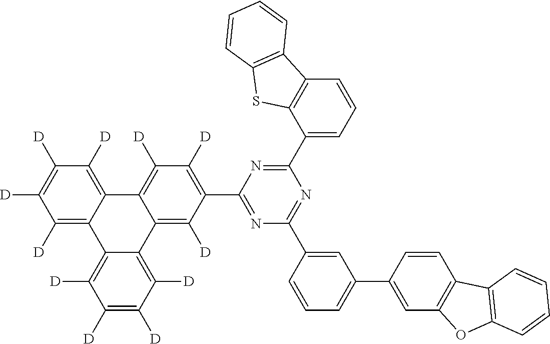 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4cccc5c4sc4ccccc45)n3)c([2H])c([2H])c21] | 59% |
| 157 | | 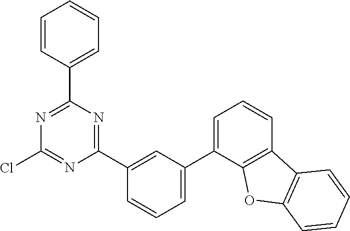 [SMILES: Clc1nc(-c2ccccc2)nc(-c2cccc(-c3cccc4c3oc3ccccc34)c2)n1] | 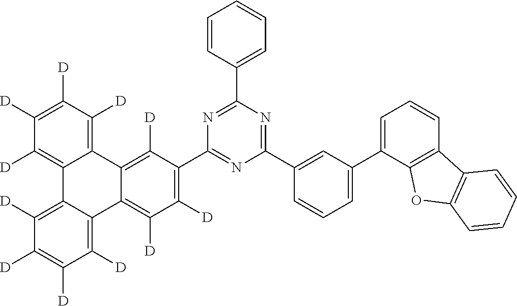 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c5oc5ccccc56)c4)n3)c([2H])c([2H])c21] | 70% |
| 159 | 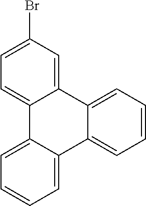 [SMILES: Brc1ccc2c3ccccc3c3ccccc3c2c1] | 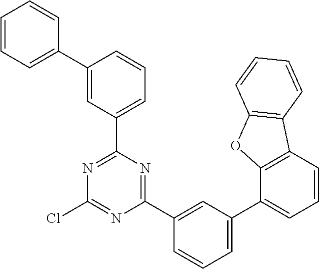 [SMILES: Clc1nc(-c2cccc(-c3ccccc3)c2)nc(-c2cccc(-c3cccc4c3oc3ccccc34)c2)n1] | 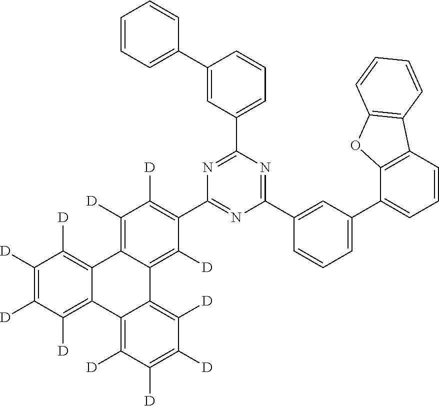 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c5oc5ccccc56)c4)n3)c([2H])c([2H])c21] | 60% |
| 162 | | 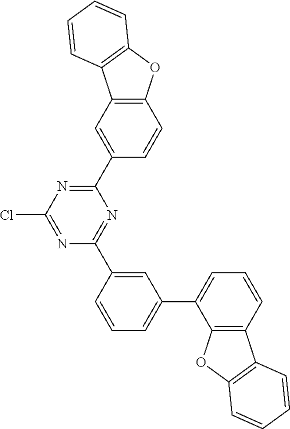 [SMILES: Clc1nc(-c2cccc(-c3cccc4c3oc3ccccc34)c2)nc(-c2ccc3oc4ccccc4c3c2)n1] | 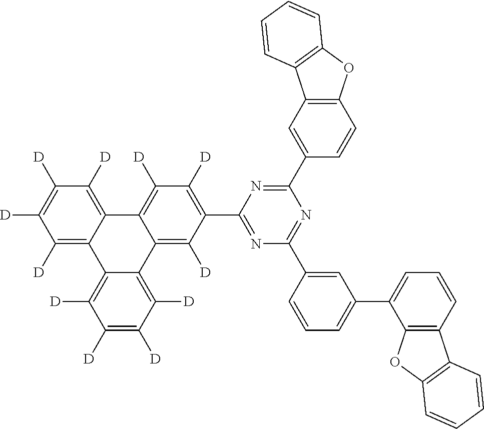 [SMILES: [2H]c1c([2H])c([2H])c2c(c1[2H])c1c([2H])c([2H])c([2H])c([2H])c1c1c([2H])c(-c3nc(-c4cccc(-c5cccc6c5oc5ccccc56)c4)nc(-c4ccc5oc6ccccc6c5c4)n3)c([2H])c([2H])c21] | 63% |
| 169 | | 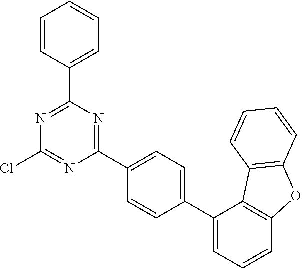 [SMILES: Clc1nc(-c2ccccc2)nc(-c2ccc(-c3cccc4oc5ccccc5c34)cc2)n1] | 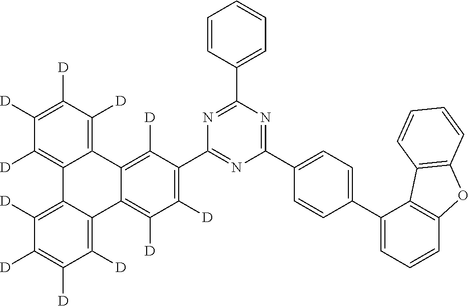 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6oc7ccccc7c56)cc4)n3)c([2H])c([2H])c21] | 53% |
| 173 | | 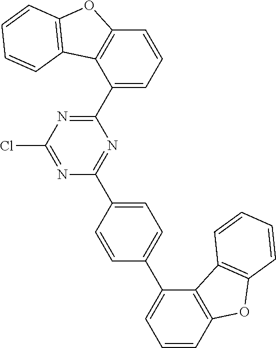 [SMILES: Clc1nc(-c2ccc(-c3cccc4oc5ccccc5c34)cc2)nc(-c2cccc3oc4ccccc4c23)n1] | 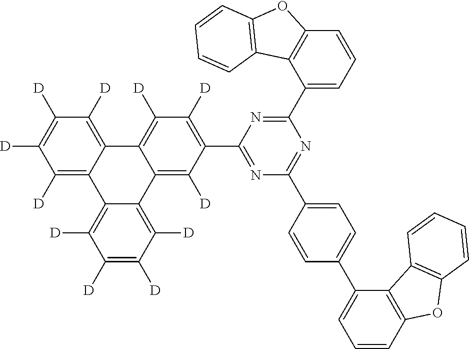 [SMILES: [2H]c1c([2H])c([2H])c2c(c1[2H])c1c([2H])c([2H])c([2H])c([2H])c1c1c([2H])c(-c3nc(-c4ccc(-c5cccc6oc7ccccc7c56)cc4)nc(-c4cccc5oc6ccccc6c45)n3)c([2H])c([2H])c21] | 67% |
| 182 | | 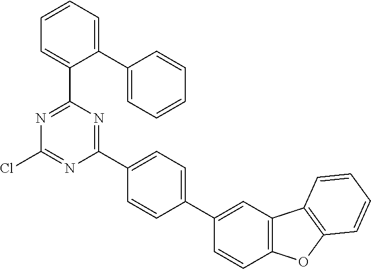 [SMILES: Clc1nc(-c2ccc(-c3ccc4oc5ccccc5c4c3)cc2)nc(-c2ccccc2-c2ccccc2)n1] | 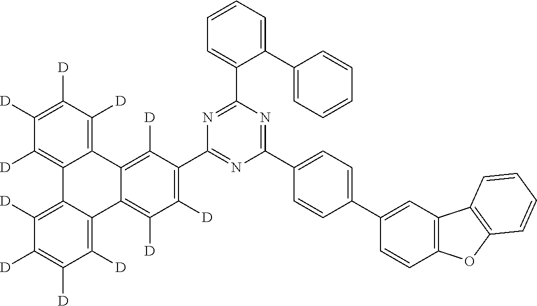 [SMILES: [2H]c1c([2H])c([2H])c2c(c1[2H])c1c([2H])c([2H])c([2H])c([2H])c1c1c([2H])c(-c3nc(-c4ccc(-c5ccc6oc7ccccc7c6c5)cc4)nc(-c4ccccc4-c4ccccc4)n3)c([2H])c([2H])c21] | 57% |
| 189 | | 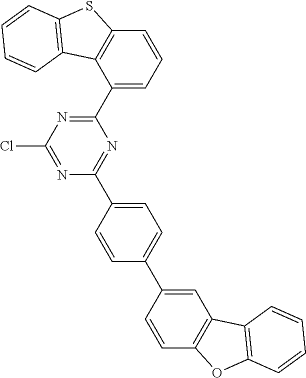 [SMILES: Clc1nc(-c2ccc(-c3ccc4oc5ccccc5c4c3)cc2)nc(-c2cccc3sc4ccccc4c23)n1] | 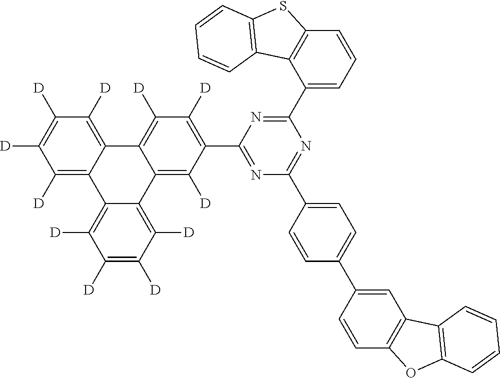 [SMILES: [2H]c1c([2H])c([2H])c2c(c1[2H])c1c([2H])c([2H])c([2H])c([2H])c1c1c([2H])c(-c3nc(-c4ccc(-c5ccc6oc7ccccc7c6c5)cc4)nc(-c4cccc5sc6ccccc6c45)n3)c([2H])c([2H])c21] | 70% |
| 194 | | 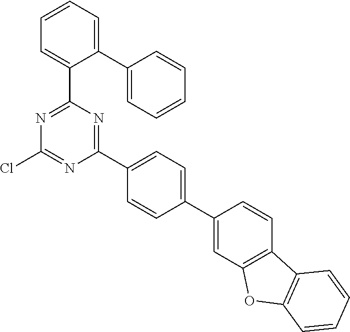 [SMILES: Clc1nc(-c2ccc(-c3ccc4c(c3)oc3ccccc34)cc2)nc(-c2ccccc2-c2ccccc2)n1] | 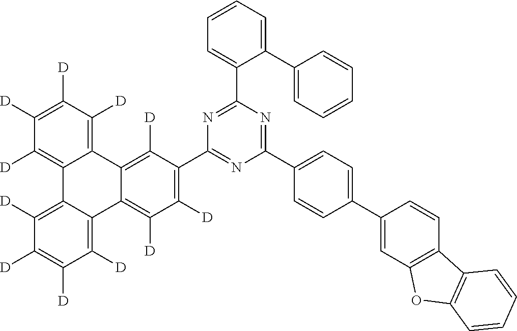 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)oc5ccccc56)cc4)nc(-c4ccccc4-c4ccccc4)n3)c([2H])c([2H])c21] | 52% |
| 195 | 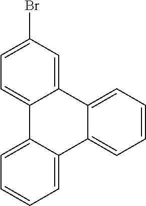 [SMILES: Brc1ccc2c3ccccc3c3ccccc3c2c1] | 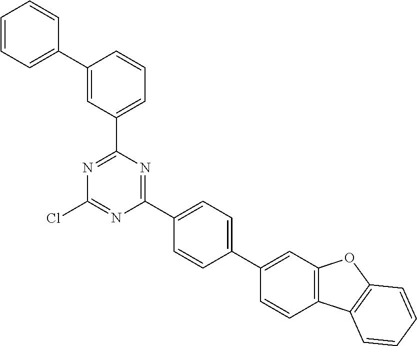 [SMILES: Clc1nc(-c2ccc(-c3ccc4c(c3)oc3ccccc34)cc2)nc(-c2cccc(-c3ccccc3)c2)n1] | 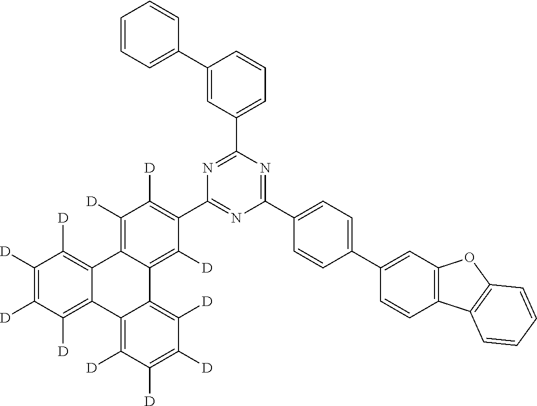 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)oc5ccccc56)cc4)nc(-c4cccc(-c5ccccc5)c4)n3)c([2H])c([2H])c21] | 68% |
| 207 | | 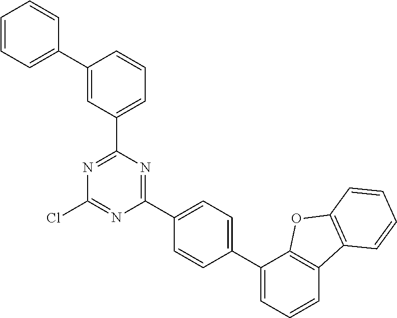 [SMILES: Clc1nc(-c2ccc(-c3cccc4c3oc3ccccc34)cc2)nc(-c2cccc(-c3ccccc3)c2)n1] | 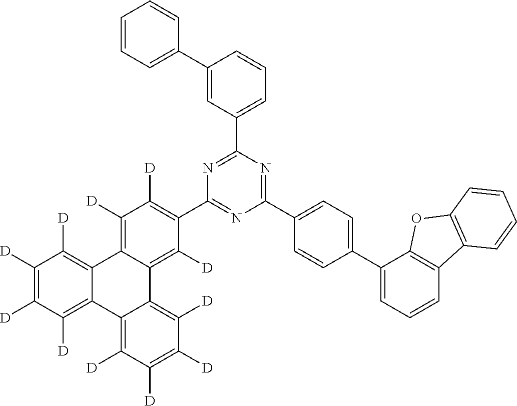 [SMILES: [2H]c1c([2H])c([2H])c2c(c1[2H])c1c([2H])c([2H])c([2H])c([2H])c1c1c([2H])c(-c3nc(-c4ccc(-c5cccc6c5oc5ccccc56)cc4)nc(-c4cccc(-c5ccccc5)c4)n3)c([2H])c([2H])c21] | 63% |
| 213 | | 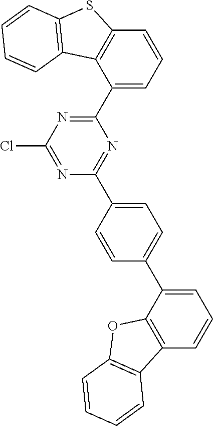 [SMILES: Clc1nc(-c2ccc(-c3cccc4c3oc3ccccc34)cc2)nc(-c2cccc3sc4ccccc4c23)n1] | 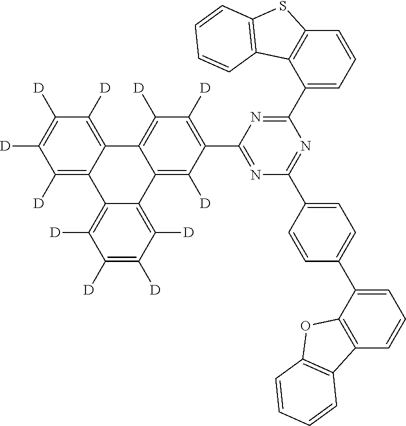 [SMILES: [2H]c1c([2H])c([2H])c2c(c1[2H])c1c([2H])c([2H])c([2H])c([2H])c1c1c([2H])c(-c3nc(-c4ccc(-c5cccc6c5oc5ccccc56)cc4)nc(-c4cccc5sc6ccccc6c45)n3)c([2H])c([2H])c21] | 57% |
| 217 | | 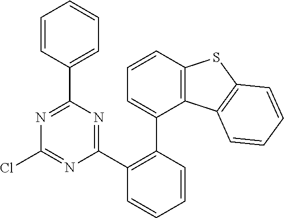 [SMILES: Clc1nc(-c2ccccc2)nc(-c2ccccc2-c2cccc3sc4ccccc4c23)n1] | 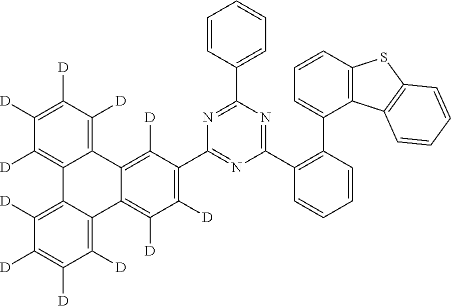 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5sc6ccccc6c45)n3)c([2H])c([2H])c21] | 64% |
| 220 | | 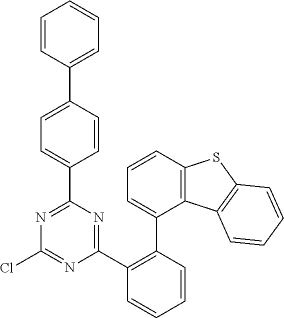 [SMILES: Clc1nc(-c2ccc(-c3ccccc3)cc2)nc(-c2ccccc2-c2cccc3sc4ccccc4c23)n1] | 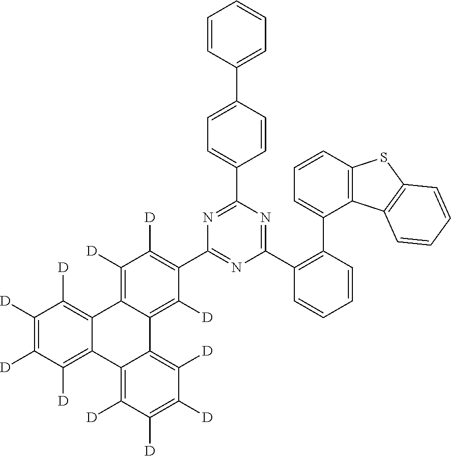 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5sc6ccccc6c45)n3)c([2H])c([2H])c21] | 59% |
| 221 | | 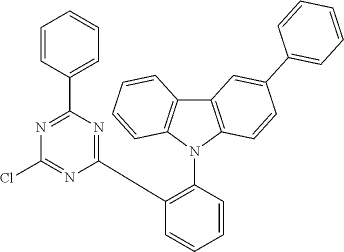 [SMILES: Clc1nc(-c2ccccc2)nc(-c2ccccc2-n2c3ccccc3c3cc(-c4ccccc4)ccc32)n1] | 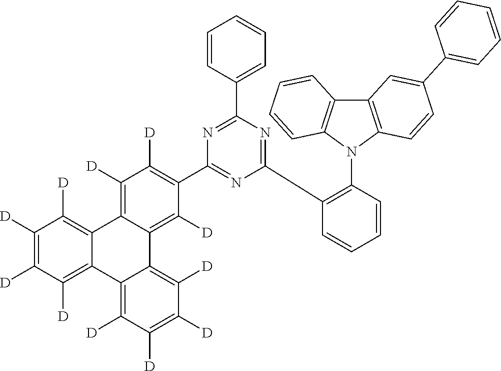 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5cc(-c6ccccc6)ccc54)n3)c([2H])c([2H])c21] | 51% |
| 224 | | 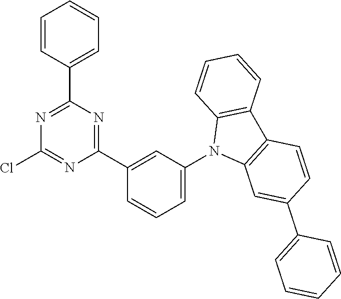 [SMILES: Clc1nc(-c2ccccc2)nc(-c2cccc(-n3c4ccccc4c4ccc(-c5ccccc5)cc43)c2)n1] | 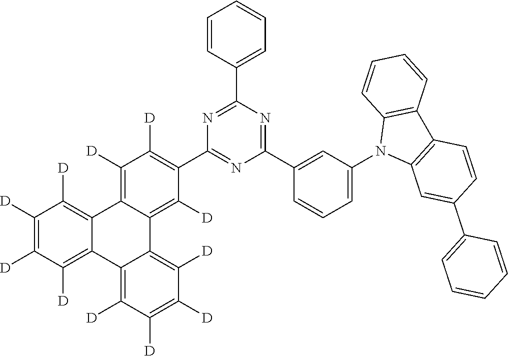 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc(-c7ccccc7)cc65)c4)n3)c([2H])c([2H])c21] | 59% |
| 226 | 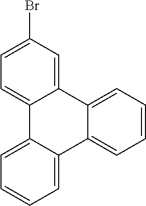 [SMILES: Brc1ccc2c3ccccc3c3ccccc3c2c1] | 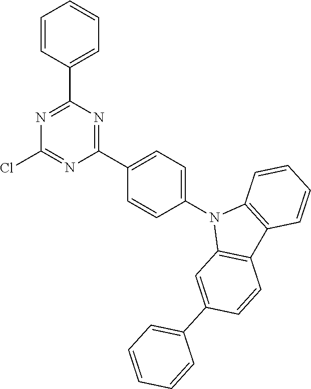 [SMILES: Clc1nc(-c2ccccc2)nc(-c2ccc(-n3c4ccccc4c4ccc(-c5ccccc5)cc43)cc2)n1] | 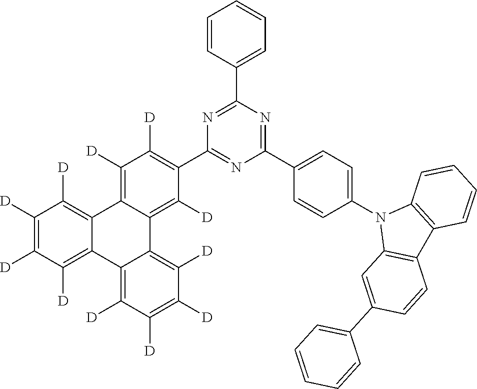 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-n5c6ccccc6c6ccc(-c7ccccc7)cc65)cc4)n3)c([2H])c([2H])c21] | 66% |
| 228 | | 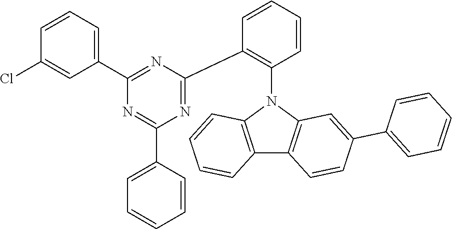 [SMILES: Clc1cccc(-c2nc(-c3ccccc3)nc(-c3ccccc3-n3c4ccccc4c4ccc(-c5ccccc5)cc43)n2)c1] | 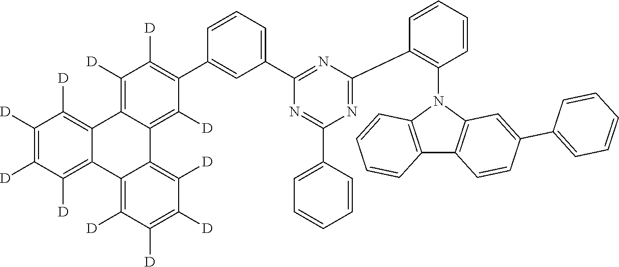 [SMILES: [2H]c1c([2H])c([2H])c2c(c1[2H])c1c([2H])c([2H])c([2H])c([2H])c1c1c([2H])c(-c3cccc(-c4nc(-c5ccccc5)nc(-c5ccccc5-n5c6ccccc6c6ccc(-c7ccccc7)cc65)n4)c3)c([2H])c([2H])c21] | 60% |
| 229 | | 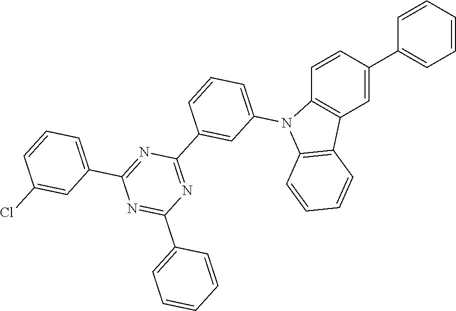 [SMILES: Clc1cccc(-c2nc(-c3ccccc3)nc(-c3cccc(-n4c5ccccc5c5cc(-c6ccccc6)ccc54)c3)n2)c1] | 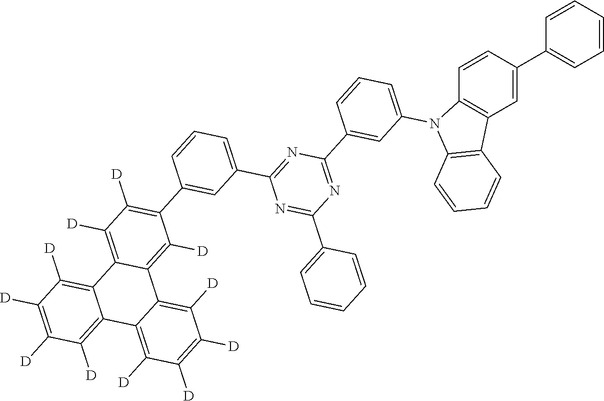 [SMILES: [2H]c1c([2H])c([2H])c2c(c1[2H])c1c([2H])c([2H])c([2H])c([2H])c1c1c([2H])c(-c3cccc(-c4nc(-c5ccccc5)nc(-c5cccc(-n6c7ccccc7c7cc(-c8ccccc8)ccc76)c5)n4)c3)c([2H])c([2H])c21] | 52% |
| 231 | | 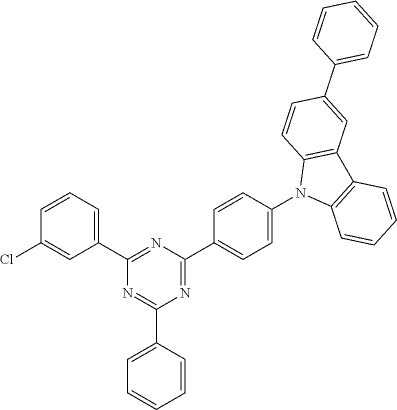 [SMILES: Clc1cccc(-c2nc(-c3ccccc3)nc(-c3ccc(-n4c5ccccc5c5cc(-c6ccccc6)ccc54)cc3)n2)c1] | 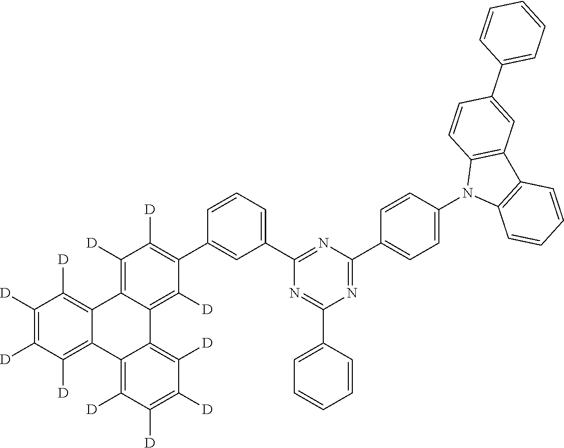 [SMILES: [2H]c1c([2H])c([2H])c2c(c1[2H])c1c([2H])c([2H])c([2H])c([2H])c1c1c([2H])c(-c3cccc(-c4nc(-c5ccccc5)nc(-c5ccc(-n6c7ccccc7c7cc(-c8ccccc8)ccc76)cc5)n4)c3)c([2H])c([2H])c21] | 51% |
| 233 | | 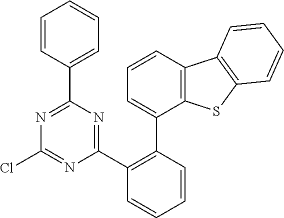 [SMILES: Clc1nc(-c2ccccc2)nc(-c2ccccc2-c2cccc3c2sc2ccccc23)n1] | 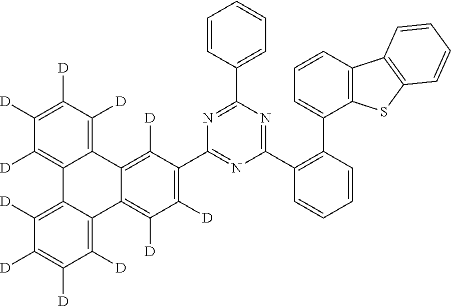 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4sc4ccccc45)n3)c([2H])c([2H])c21] | 64% |
| 239 | | 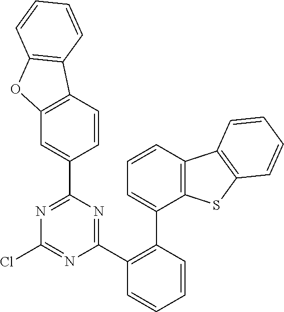 [SMILES: Clc1nc(-c2ccc3c(c2)oc2ccccc23)nc(-c2ccccc2-c2cccc3c2sc2ccccc23)n1] | 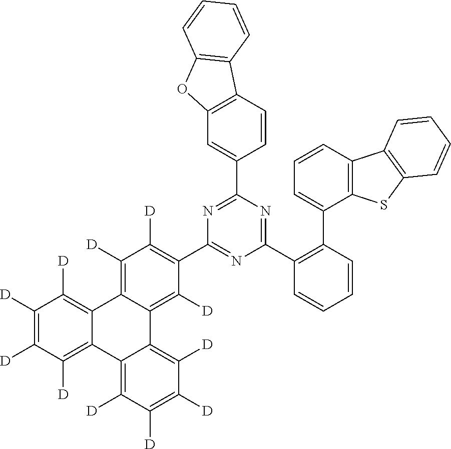 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c4sc4ccccc45)n3)c([2H])c([2H])c21] | 52% |
| 241 | | 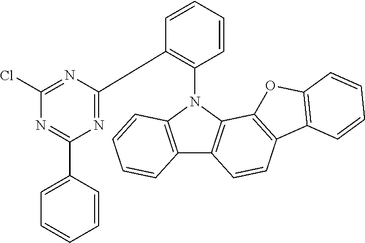 [SMILES: Clc1nc(-c2ccccc2)nc(-c2ccccc2-n2c3ccccc3c3ccc4c5ccccc5oc4c32)n1] | 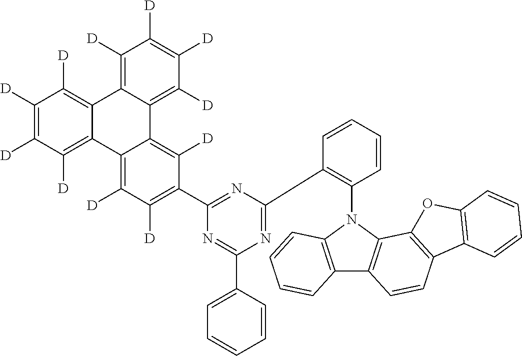 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7ccccc7oc6c54)n3)c([2H])c([2H])c21] | 63% |
| 242 | 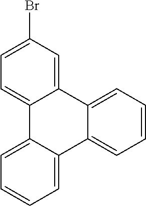 [SMILES: Brc1ccc2c3ccccc3c3ccccc3c2c1] | 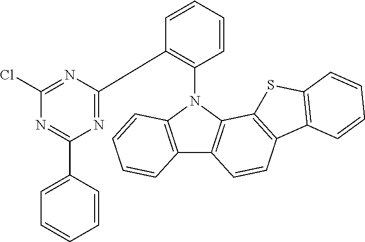 [SMILES: Clc1nc(-c2ccccc2)nc(-c2ccccc2-n2c3ccccc3c3ccc4c5ccccc5sc4c32)n1] | 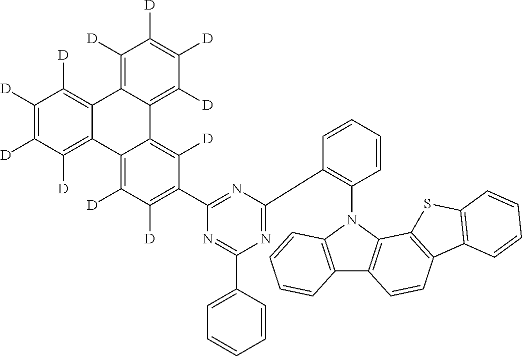 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7ccccc7sc6c54)n3)c([2H])c([2H])c21] | 61% |
| 243 | | 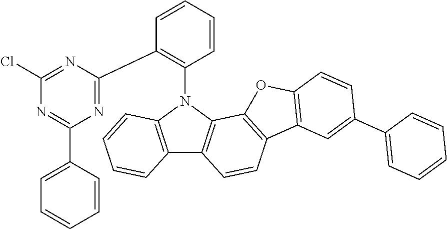 [SMILES: Clc1nc(-c2ccccc2)nc(-c2ccccc2-n2c3ccccc3c3ccc4c5cc(-c6ccccc6)ccc5oc4c32)n1] | 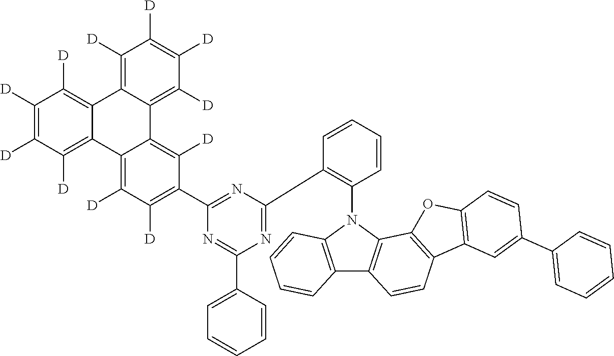 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7cc(-c8ccccc8)ccc7oc6c54)n3)c([2H])c([2H])c21] | 65% |
| 245 | | 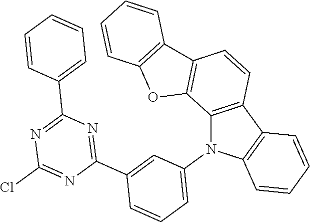 [SMILES: Clc1nc(-c2ccccc2)nc(-c2cccc(-n3c4ccccc4c4ccc5c6ccccc6oc5c43)c2)n1] | 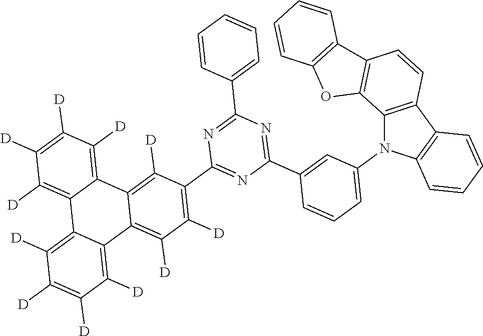 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8oc7c65)c4)n3)c([2H])c([2H])c21] | 60% |
| 248 | | 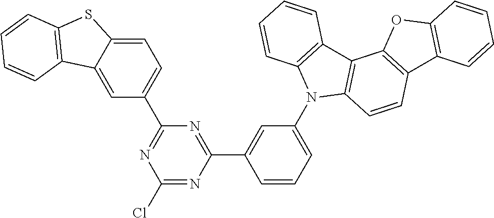 [SMILES: Clc1nc(-c2cccc(-n3c4ccccc4c4c5oc6ccccc6c5ccc43)c2)nc(-c2ccc3sc4ccccc4c3c2)n1] | 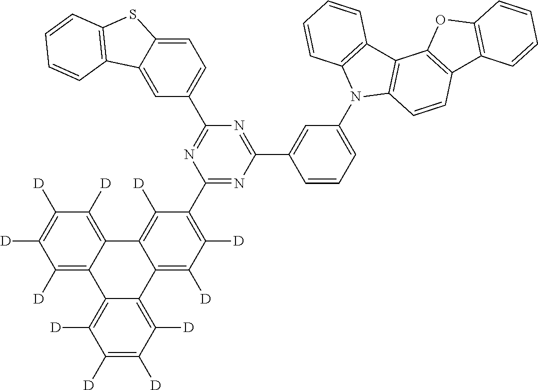 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6c7oc8ccccc8c7ccc65)c4)nc(-c4ccc5sc6ccccc6c5c4)n3)c([2H])c([2H])c21] | 64% |
| 250 | | 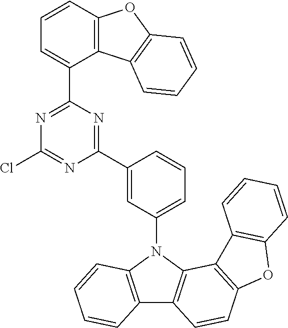 [SMILES: Clc1nc(-c2cccc(-n3c4ccccc4c4ccc5oc6ccccc6c5c43)c2)nc(-c2cccc3oc4ccccc4c23)n1] | 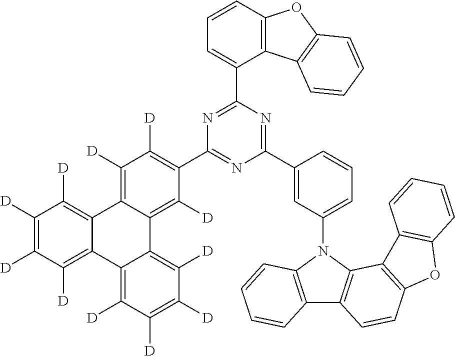 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccc7oc8ccccc8c7c65)c4)nc(-c4cccc5oc6ccccc6c45)n3)c([2H])c([2H])c21] | 57% |
| 257 | | 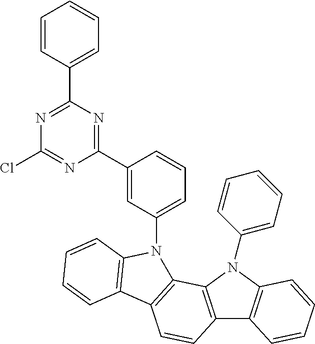 [SMILES: Clc1nc(-c2ccccc2)nc(-c2cccc(-n3c4ccccc4c4ccc5c6ccccc6n(-c6ccccc6)c5c43)c2)n1] | 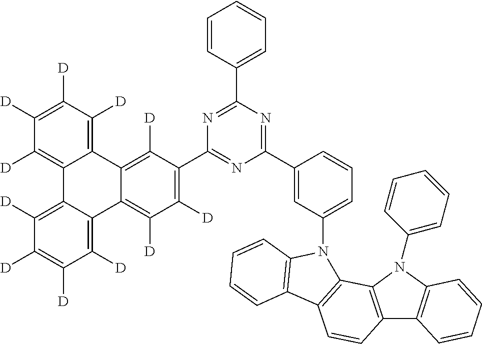 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8n(-c8ccccc8)c7c65)c4)n3)c([2H])c([2H])c21] | 52% |
| 260 | | 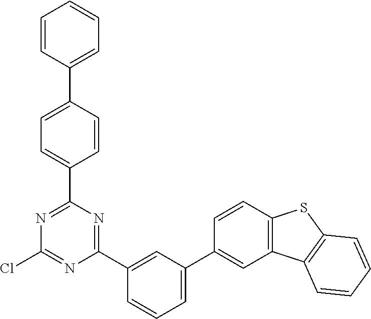 [SMILES: Clc1nc(-c2ccc(-c3ccccc3)cc2)nc(-c2cccc(-c3ccc4sc5ccccc5c4c3)c2)n1] | 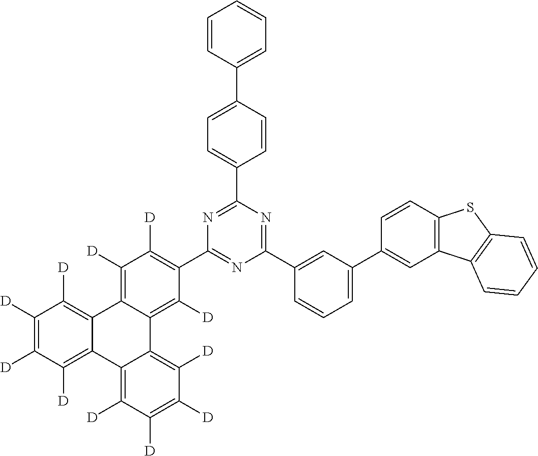 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6sc7ccccc7c6c5)c4)n3)c([2H])c([2H])c21] | 64% |
| 276 | 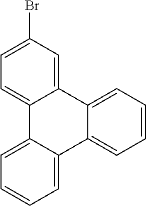 [SMILES: Brc1ccc2c3ccccc3c3ccccc3c2c1] | 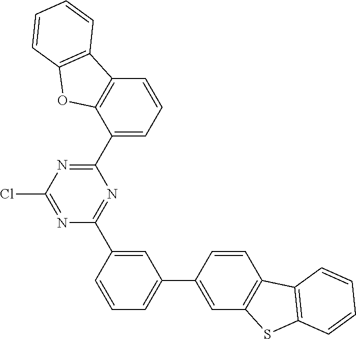 [SMILES: Clc1nc(-c2cccc(-c3ccc4c(c3)sc3ccccc34)c2)nc(-c2cccc3c2oc2ccccc23)n1] | 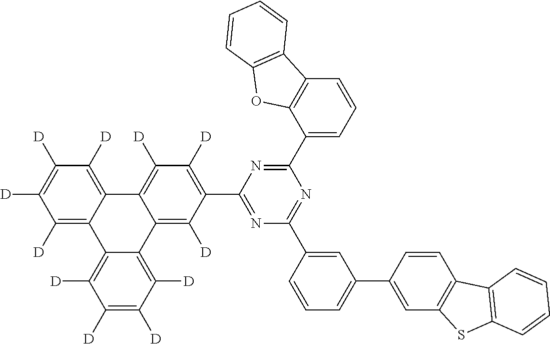 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4cccc5c4oc4ccccc45)n3)c([2H])c([2H])c21] | 58% |
| 281 | | 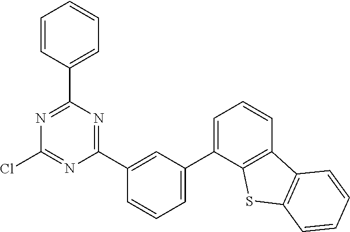 [SMILES: Clc1nc(-c2ccccc2)nc(-c2cccc(-c3cccc4c3sc3ccccc34)c2)n1] | 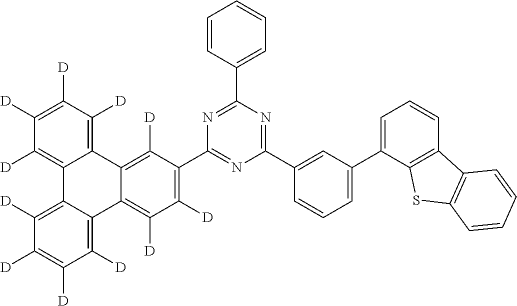 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c5sc5ccccc56)c4)n3)c([2H])c([2H])c21] | 57% |
| 288 | | 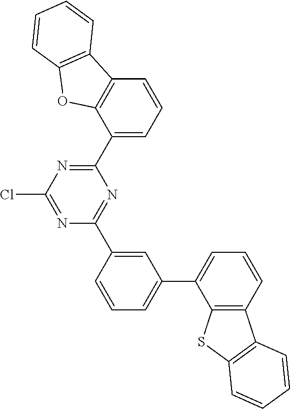 [SMILES: Clc1nc(-c2cccc(-c3cccc4c3sc3ccccc34)c2)nc(-c2cccc3c2oc2ccccc23)n1] | 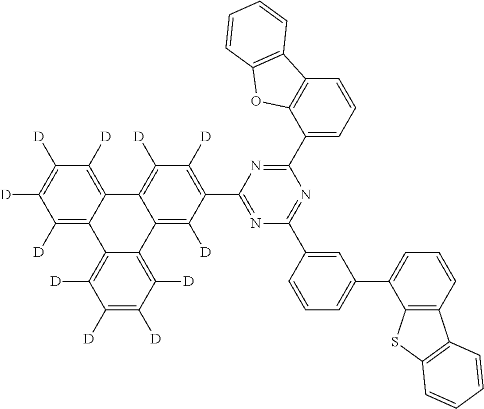 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5c4oc4ccccc45)n3)c([2H])c([2H])c21] | 63% |
| 295 | | 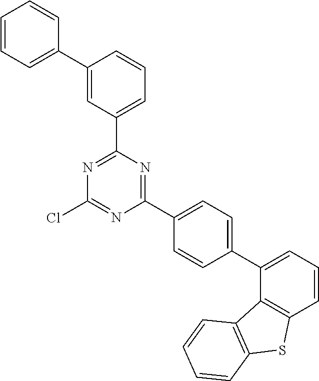 [SMILES: Clc1nc(-c2ccc(-c3cccc4sc5ccccc5c34)cc2)nc(-c2cccc(-c3ccccc3)c2)n1] | 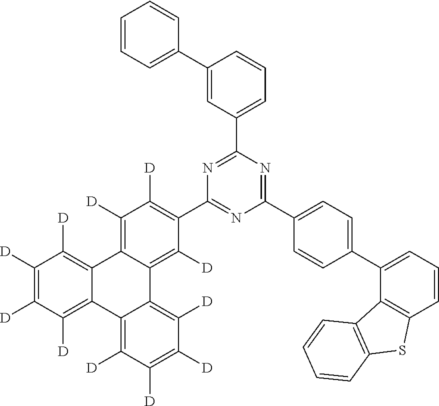 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc(-c5ccccc5)c4)n3)c([2H])c([2H])c21] | 66% |
| 301 | | 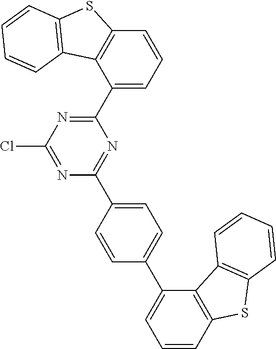 [SMILES: Clc1nc(-c2ccc(-c3cccc4sc5ccccc5c34)cc2)nc(-c2cccc3sc4ccccc4c23)n1] | 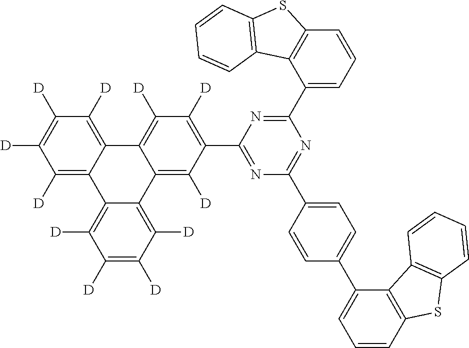 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5sc6ccccc6c45)n3)c([2H])c([2H])c21] | 55% |
| 307 | | 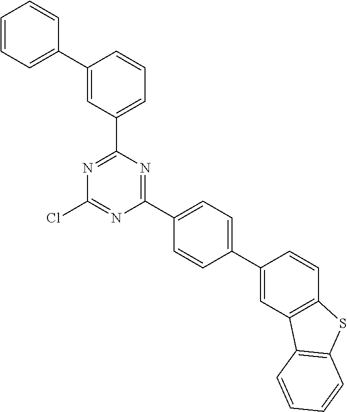 [SMILES: Clc1nc(-c2ccc(-c3ccc4sc5ccccc5c4c3)cc2)nc(-c2cccc(-c3ccccc3)c2)n1] | 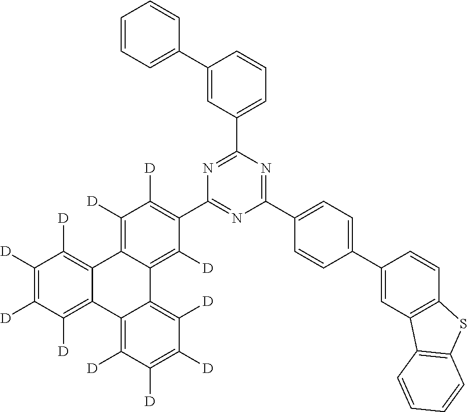 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc(-c5ccccc5)c4)n3)c([2H])c([2H])c21] | 54% |
| 313 | | 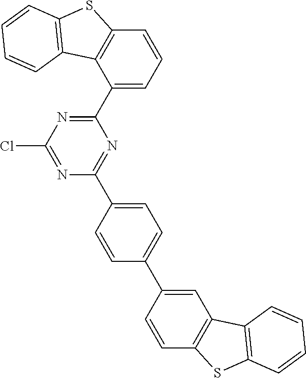 [SMILES: Clc1nc(-c2ccc(-c3ccc4sc5ccccc5c4c3)cc2)nc(-c2cccc3sc4ccccc4c23)n1] | 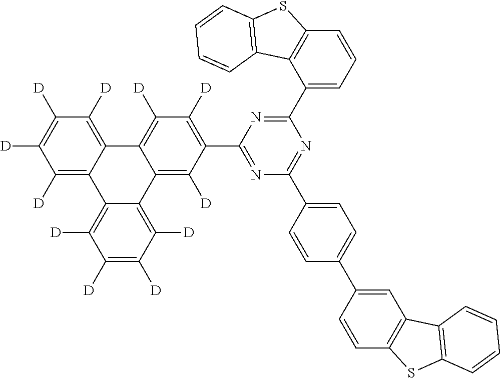 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5sc6ccccc6c45)n3)c([2H])c([2H])c21] | 53% |
| 318 | 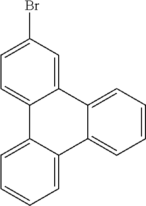 [SMILES: Brc1ccc2c3ccccc3c3ccccc3c2c1] | 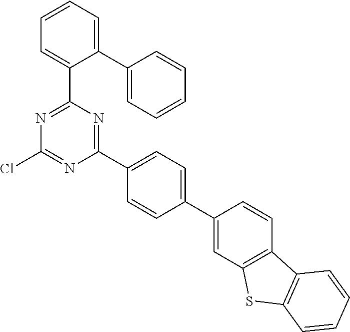 [SMILES: Clc1nc(-c2ccc(-c3ccc4c(c3)sc3ccccc34)cc2)nc(-c2ccccc2-c2ccccc2)n1] | 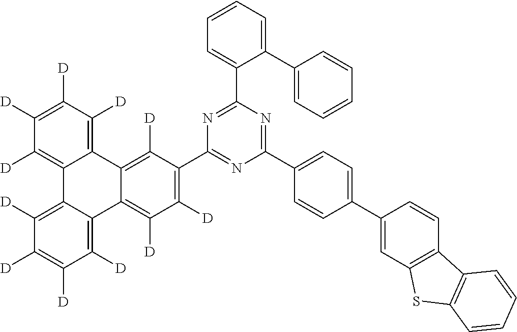 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccccc4-c4ccccc4)n3)c([2H])c([2H])c21] | 69% |
| 329 | | 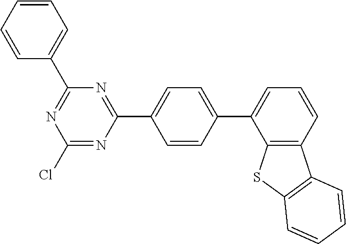 [SMILES: Clc1nc(-c2ccccc2)nc(-c2ccc(-c3cccc4c3sc3ccccc34)cc2)n1] | 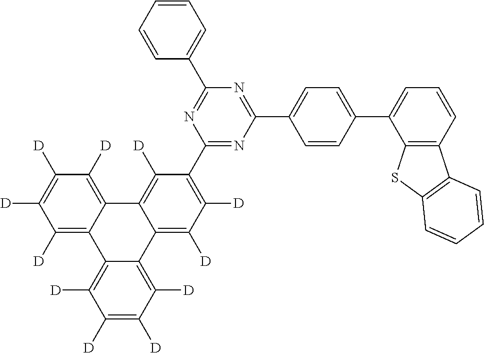 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5sc5ccccc56)cc4)n3)c([2H])c([2H])c21] | 69% |
| 337 | | 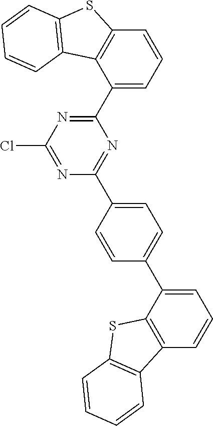 [SMILES: Clc1nc(-c2ccc(-c3cccc4c3sc3ccccc34)cc2)nc(-c2cccc3sc4ccccc4c23)n1] | 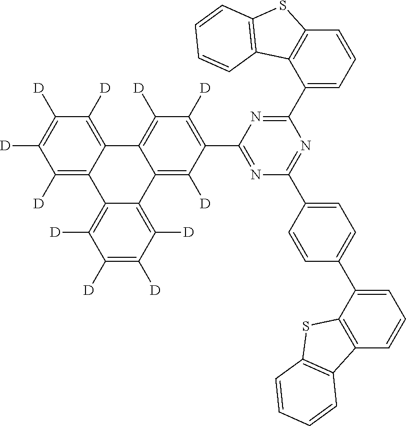 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5sc6ccccc6c45)n3)c([2H])c([2H])c21] | 61% |
| 344 | | 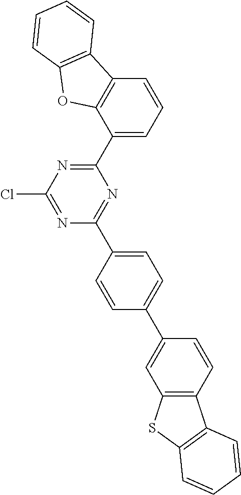 [SMILES: Clc1nc(-c2ccc(-c3ccc4c(c3)sc3ccccc34)cc2)nc(-c2cccc3c2oc2ccccc23)n1] | 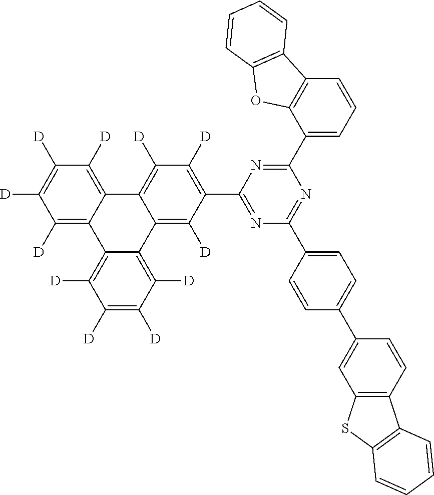 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4oc4ccccc45)n3)c([2H])c([2H])c21] | 69% |
| 349 | | 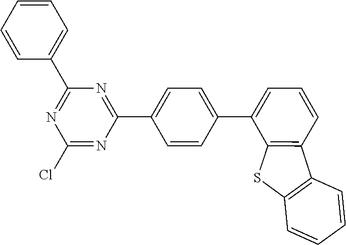 [SMILES: Clc1nc(-c2ccccc2)nc(-c2ccc(-c3cccc4c3sc3ccccc34)cc2)n1] | 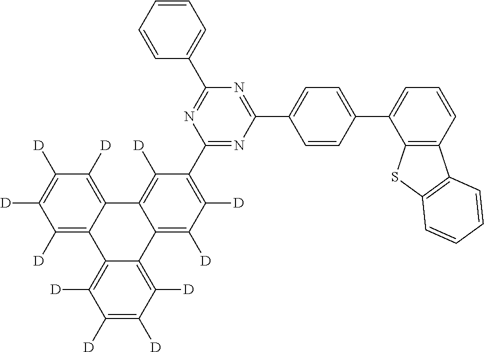 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5sc5ccccc56)cc4)n3)c([2H])c([2H])c21] | 53% |
| 357 | | 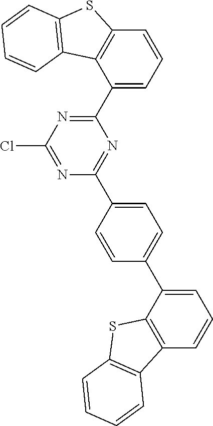 [SMILES: Clc1nc(-c2ccc(-c3cccc4c3sc3ccccc34)cc2)nc(-c2cccc3sc4ccccc4c23)n1] | 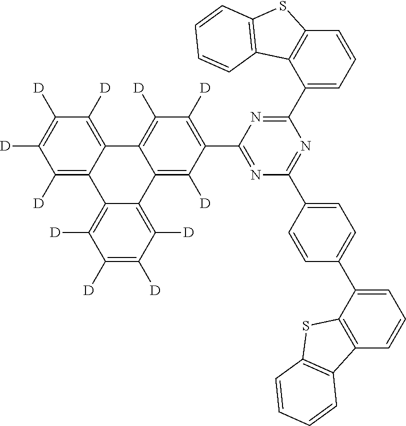 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5sc6ccccc6c45)n3)c([2H])c([2H])c21] | 65% |
| 361 | | 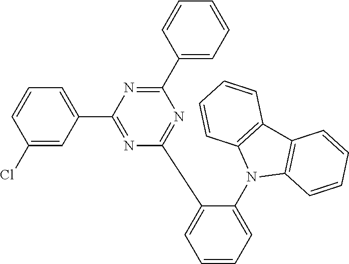 [SMILES: Clc1cccc(-c2nc(-c3ccccc3)nc(-c3ccccc3-n3c4ccccc4c4ccccc43)n2)c1] | 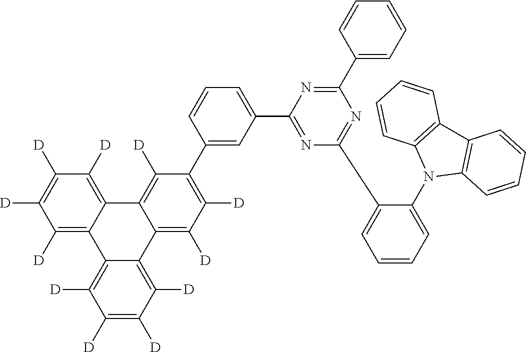 [SMILES: [2H]c1c([2H])c([2H])c2c(c1[2H])c1c([2H])c([2H])c([2H])c([2H])c1c1c([2H])c(-c3cccc(-c4nc(-c5ccccc5)nc(-c5ccccc5-n5c6ccccc6c6ccccc65)n4)c3)c([2H])c([2H])c21] | 61% |
| 396 | 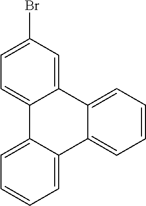 [SMILES: Brc1ccc2c3ccccc3c3ccccc3c2c1] | 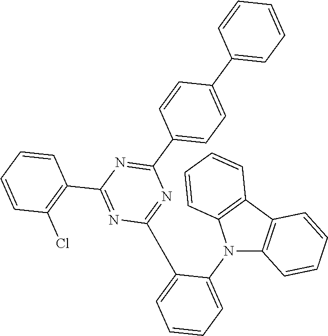 [SMILES: Clc1ccccc1-c1nc(-c2ccc(-c3ccccc3)cc2)nc(-c2ccccc2-n2c3ccccc3c3ccccc32)n1] | 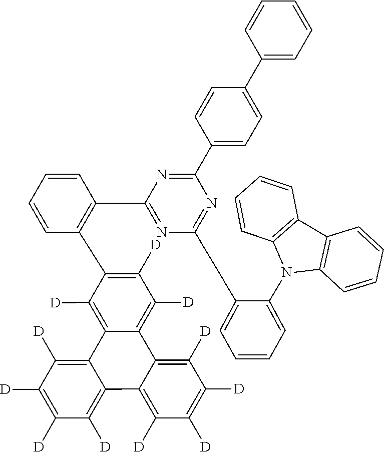 [SMILES: [2H]c1c([2H])c([2H])c2c(c1[2H])c1c([2H])c([2H])c([2H])c([2H])c1c1c([2H])c(-c3ccccc3-c3nc(-c4ccc(-c5ccccc5)cc4)nc(-c4ccccc4-n4c5ccccc5c5ccccc54)n3)c([2H])c([2H])c21] | 59% |
| 402 | | 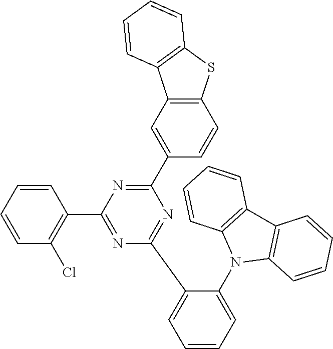 [SMILES: Clc1ccccc1-c1nc(-c2ccc3sc4ccccc4c3c2)nc(-c2ccccc2-n2c3ccccc3c3ccccc32)n1] | 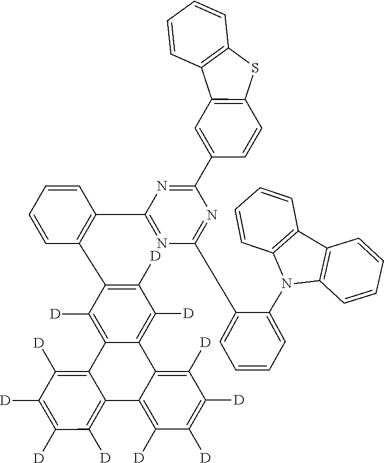 [SMILES: [2H]c1c([2H])c([2H])c2c(c1[2H])c1c([2H])c([2H])c([2H])c([2H])c1c1c([2H])c(-c3ccccc3-c3nc(-c4ccc5sc6ccccc6c5c4)nc(-c4ccccc4-n4c5ccccc5c5ccccc54)n3)c([2H])c([2H])c21] | 53% |
| 405 | | 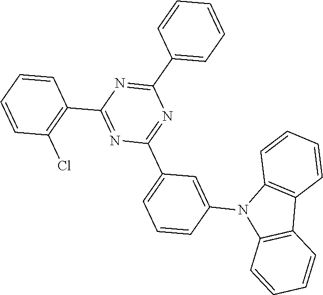 [SMILES: Clc1ccccc1-c1nc(-c2ccccc2)nc(-c2cccc(-n3c4ccccc4c4ccccc43)c2)n1] | 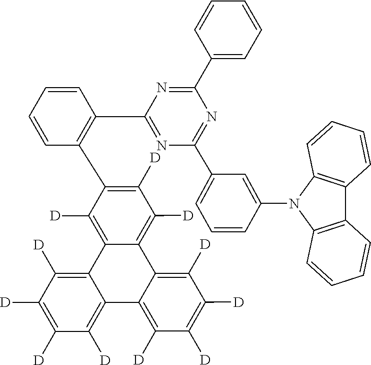 [SMILES: [2H]c1c([2H])c([2H])c2c(c1[2H])c1c([2H])c([2H])c([2H])c([2H])c1c1c([2H])c(-c3ccccc3-c3nc(-c4ccccc4)nc(-c4cccc(-n5c6ccccc6c6ccccc65)c4)n3)c([2H])c([2H])c21] | 60% |
| 406 | | 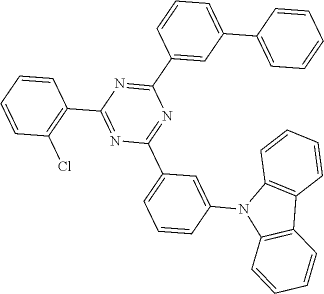 [SMILES: Clc1ccccc1-c1nc(-c2cccc(-c3ccccc3)c2)nc(-c2cccc(-n3c4ccccc4c4ccccc43)c2)n1] | 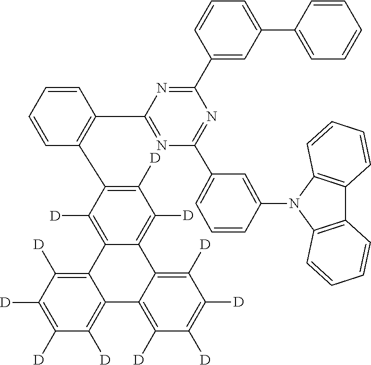 [SMILES: [2H]c1c([2H])c([2H])c2c(c1[2H])c1c([2H])c([2H])c([2H])c([2H])c1c1c([2H])c(-c3ccccc3-c3nc(-c4cccc(-c5ccccc5)c4)nc(-c4cccc(-n5c6ccccc6c6ccccc65)c4)n3)c([2H])c([2H])c21] | 57% |
| 413 | | 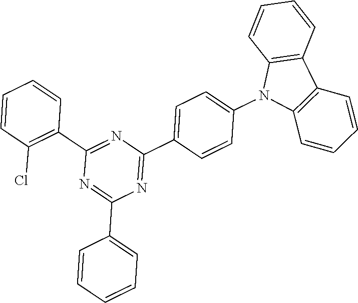 [SMILES: Clc1ccccc1-c1nc(-c2ccccc2)nc(-c2ccc(-n3c4ccccc4c4ccccc43)cc2)n1] | 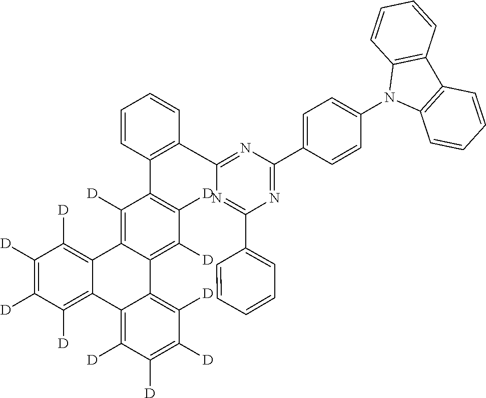 [SMILES: [2H]c1c([2H])c([2H])c2c(c1[2H])c1c([2H])c([2H])c([2H])c([2H])c1c1c([2H])c(-c3ccccc3-c3nc(-c4ccccc4)nc(-c4ccc(-n5c6ccccc6c6ccccc65)cc4)n3)c([2H])c([2H])c21] | 59% |
| 415 | | 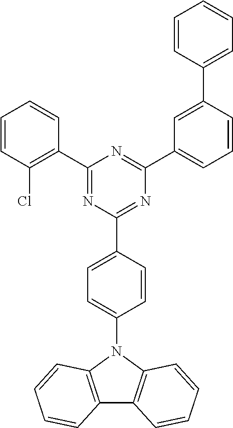 [SMILES: Clc1ccccc1-c1nc(-c2ccc(-n3c4ccccc4c4ccccc43)cc2)nc(-c2cccc(-c3ccccc3)c2)n1] | 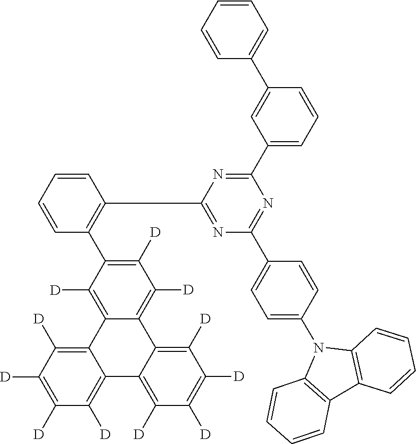 [SMILES: [2H]c1c([2H])c([2H])c2c(c1[2H])c1c([2H])c([2H])c([2H])c([2H])c1c1c([2H])c(-c3ccccc3-c3nc(-c4ccc(-n5c6ccccc6c6ccccc65)cc4)nc(-c4cccc(-c5ccccc5)c4)n3)c([2H])c([2H])c21] | 53% |
| 417 | 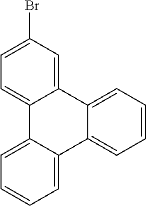 [SMILES: Brc1ccc2c3ccccc3c3ccccc3c2c1] | 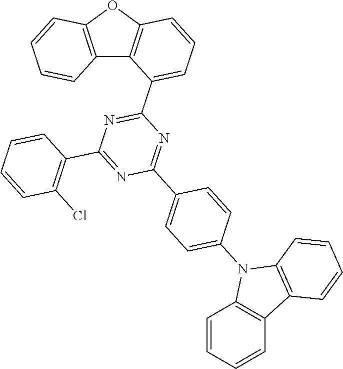 [SMILES: Clc1ccccc1-c1nc(-c2ccc(-n3c4ccccc4c4ccccc43)cc2)nc(-c2cccc3oc4ccccc4c23)n1] | 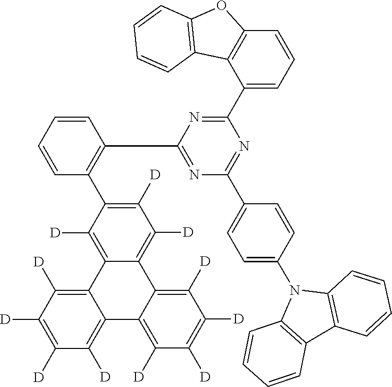 [SMILES: [2H]c1c([2H])c([2H])c2c(c1[2H])c1c([2H])c([2H])c([2H])c([2H])c1c1c([2H])c(-c3ccccc3-c3nc(-c4ccc(-n5c6ccccc6c6ccccc65)cc4)nc(-c4cccc5oc6ccccc6c45)n3)c([2H])c([2H])c21] | 58% |
| 420 | | 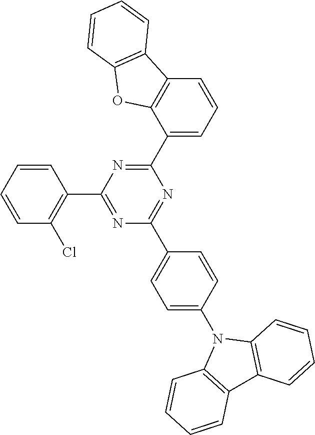 [SMILES: Clc1ccccc1-c1nc(-c2ccc(-n3c4ccccc4c4ccccc43)cc2)nc(-c2cccc3c2oc2ccccc23)n1] | 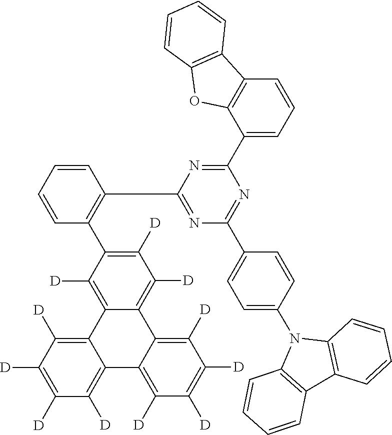 [SMILES: [2H]c1c([2H])c([2H])c2c(c1[2H])c1c([2H])c([2H])c([2H])c([2H])c1c1c([2H])c(-c3ccccc3-c3nc(-c4ccc(-n5c6ccccc6c6ccccc65)cc4)nc(-c4cccc5c4oc4ccccc45)n3)c([2H])c([2H])c21] | 51% |
| 421 | | 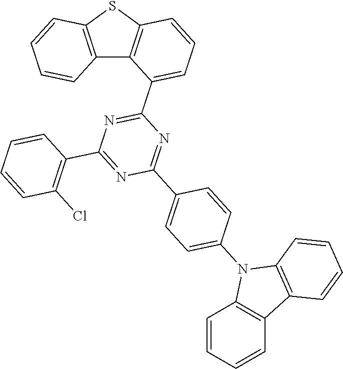 [SMILES: Clc1ccccc1-c1nc(-c2ccc(-n3c4ccccc4c4ccccc43)cc2)nc(-c2cccc3sc4ccccc4c23)n1] | 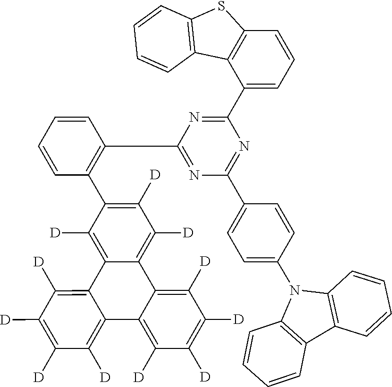 [SMILES: [2H]c1c([2H])c([2H])c2c(c1[2H])c1c([2H])c([2H])c([2H])c([2H])c1c1c([2H])c(-c3ccccc3-c3nc(-c4ccc(-n5c6ccccc6c6ccccc65)cc4)nc(-c4cccc5sc6ccccc6c45)n3)c([2H])c([2H])c21] | 53% |
| 503 | 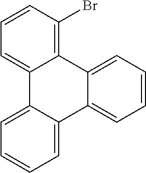 [SMILES: Brc1cccc2c3ccccc3c3ccccc3c12] | 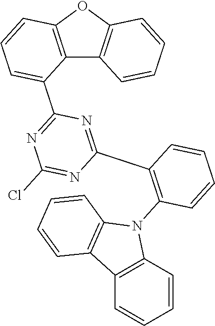 [SMILES: Clc1nc(-c2ccccc2-n2c3ccccc3c3ccccc32)nc(-c2cccc3oc4ccccc4c23)n1] | 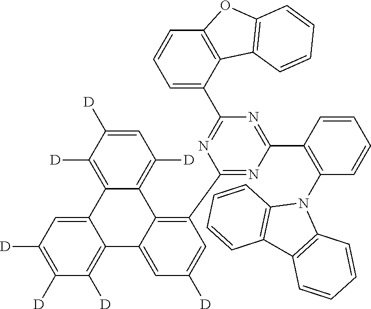 [SMILES: [2H]c1cc(-c2nc(-c3ccccc3-n3c4ccccc4c4ccccc43)nc(-c3cccc4oc5ccccc5c34)n2)c2c(c1)c1c([2H])c([2H])c([2H])cc1c1c([2H])c([2H])cc([2H])c21] | 54% |
| 511 | 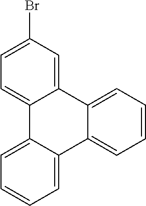 [SMILES: Brc1ccc2c3ccccc3c3ccccc3c2c1] | 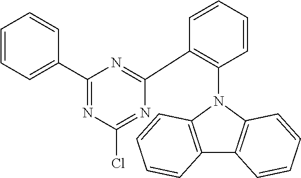 [SMILES: Clc1nc(-c2ccccc2)nc(-c2ccccc2-n2c3ccccc3c3ccccc32)n1] | 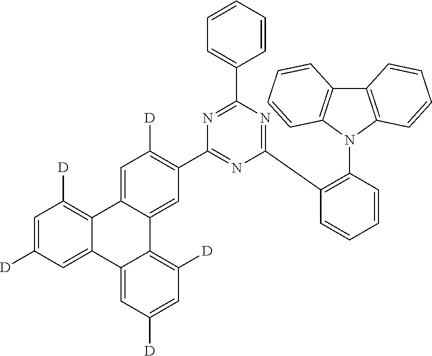 [SMILES: [2H]c1cc([2H])c2c(c1)c1cc([2H])cc([2H])c1c1cc(-c3nc(-c4ccccc4)nc(-c4ccccc4-n4c5ccccc5c5ccccc54)n3)c([2H])cc12] | 59% |
| 516 | 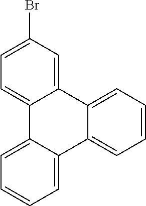 [SMILES: Brc1ccc2c3ccccc3c3ccccc3c2c1] | 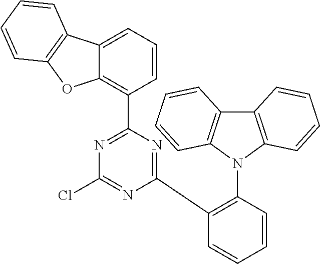 [SMILES: Clc1nc(-c2ccccc2-n2c3ccccc3c3ccccc32)nc(-c2cccc3c2oc2ccccc23)n1] | 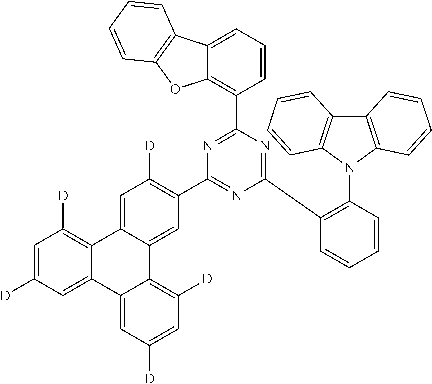 [SMILES: [2H]c1cc([2H])c2c(c1)c1cc([2H])cc([2H])c1c1cc(-c3nc(-c4ccccc4-n4c5ccccc5c5ccccc54)nc(-c4cccc5c4oc4ccccc45)n3)c([2H])cc12] | 56% |

Compounds were prepared in the same manner as in the Preparation Examples, and the synthesis confirmation results thereof are shown in the following Tables 2 and 3. Table 2 shows the measured values of ¹H NMR (CDCl₃, 400 Mz), and Table 3 shows the measured values of field desorption mass spectrometry (FD-MS).

TABLE 2

| Compound | |
|---|---|
| No. | ¹H NMR(CDCl₃, 400 MHz) |
| 1 | δ = 8.55 (1H, dd), 8.36-8.32 (2H, m), 8.19 (1H, dd), 7.94-7.91 (3H, m), 7.80 |
| | (1H, td), 7.58-7.46 (6H, m), 7.35 (1H, td), 7.20-7.16 (2H, m) |
| 4 | δ = 8.53 (1H, dd), 8.19 (1H, dd), 7.96-7.91 (5H, m), 7.80-7.75 (3H, m), |
| | 7.58-7.35 (7H, m), 7.25-7.16 (4H, m) |
| 5 | δ = 8.54 (1H, dd), 8.17 (1H, dd), 7.98-7.80 (6H, m), 7.69 (1H, dd), 7.57-7.31 |
| | (8H, m), 7.21-7.17 (2H, m) |
| 7 | δ = 8.50 (1H, dd), 8.14 (1H, dd), 7.95-7.82 (6H, m), 7.74 (1H, dd), 7.60-7.34 |
| | (8H, m), 7.23-7.19 (2H, m) |
| 10 | δ = 8.53 (1H, dd), 8.20 (1H, dd), 7.99-7.85 (5H, m), 7.75-7.69 (2H, dd), |
| | 7.62-7.37 (8H, m), 7.20-7.15 (2H, m) |
| 12 | δ = 8.51 (1H, dd), 8.18 (1H, dd), 8.01-7.83 (6H, m), 7.71 (1H, dd), 7.58-7.35 |
| | (8H, m), 7.19-7.16 (2H, m) |
| 14 | δ = 8.49 (1H, dd), 8.22 (1H, dd), 8.00-7.93 (5H, m), 7.77-7.69 (3H, m), |
| | 7.60-7.37 (7H, m), 7.27-7.19 (4H, m) |
| 15 | δ = 8.50 (1H, dd), 8.21 (1H, dd), 7.99-7.88 (5H, m), 7.75-7.63 (3H, m), |
| | 7.57-7.38 (7H, m), 7.23-7.18 (4H, m) |
| 18 | δ = 8.52 (1H, dd), 8.20 (1H, dd), 8.03-7.84 (6H, m), 7.71 (1H, dd), 7.60-7.36 |
| | (8H, m), 7.21-7.15 (2H, m) |
| 20 | δ = 8.51 (1H, dd), 8.22 (1H, dd), 7.98-7.85 (6H, m), 7.68 (1H, dd), 7.61-7.39 |
| | (8H, m), 7.22-7.17 (2H, m) |
| 21 | δ = 8.52 (1H, dd), 8.17 (1H, dd), 8.02-7.88 (6H, m), 7.72 (1H, dd), 7.63-7.41 |
| | (8H, m), 7.21-7.18 (2H, m) |
| 23 | δ = 8.50 (1H, dd), 8.16 (1H, dd), 7.97-7.85 (6H, m), 7.69 (1H, dd), 7.60-7.38 |
| | (8H, m), 7.21-7.14 (2H, m) |
| 25 | δ = 8.56 (1H, dd), 8.40-8.33 (2H, m), 8.16 (1H, dd), 8.01-7.90 (3H, m), 7.78 |
| | (1H, td), 7.61-7.47 (6H, m), 7.38 (1H, td), 7.22-7.17 (2H, m) |
| 29 | δ = 8.53 (1H, dd), 8.21 (1H, dd), 7.97-7.83 (6H, m), 7.72 (1H, dd), 7.61-7.36 |
| | (8H, m), 7.19-7.15 (2H, m) |
| 37 | δ = 8.53 (1H, dd), 8.39-8.32 (2H, m), 8.18 (1H, dd), 7.99-7.89 (3H, m), 7.81 |
| | (1H, td), 7.62-7.45 (6H, m), 7.41 (1H, td), 7.20-7.16 (2H, m) |
| 39 | δ = 8.51 (1H, dd), 8.21 (1H, dd), 7.98-7.90 (5H, m), 7.81-7.72 (3H, m), |
| | 7.61-7.39 (7H, m), 7.23-7.14 (4H, m) |
| 41 | δ = 8.51 (1H, dd), 8.20 (1H, dd), 7.99-7.80 (6H, m), 7.63 (1H, dd), 7.55-7.37 |
| | (8H, m), 7.20-7.17 (2H, m) |
| 42 | δ = 8.50 (1H, dd), 8.22 (1H, dd), 8.01-7.83 (6H, m), 7.65 (1H, dd), 7.59-7.38 |
| | (8H, m), 7.22-7.19 (2H, m) |
| 43 | δ = 8.54 (1H, dd), 8.19 (1H, dd), 7.97-7.82 (6H, m), 7.69 (1H, dd), 7.61-7.39 |
| | (8H, m), 7.21-7.18 (2H, m) |
| 44 | δ = 8.51 (1H, dd), 8.18 (1H, dd), 8.00-7.79 (6H, m), 7.66 (1H, dd), 7.58-7.38 |
| | (8H, m), 7.21-7.18 (2H, m) |
| 46 | δ = 8.56 (1H, dd), 8.22 (1H, dd), 7.97-7.83 (5H, m), 7.76-7.71 (2H, dd), |
| | 7.65-7.39 (8H, m), 7.21-7.17 (2H, m) |
| 49 | δ = 8.56 (1H, dd), 8.37-8.31 (2H, m), 8.18 (1H, dd), 7.98-7.93 (3H, m), 7.81 |
| | (1H, td), 7.61-7.49 (6H, m), 7.41 (1H, td), 7.21-7.18 (2H, m) |
| 50 | δ = 8.57 (1H, dd), 8.23 (1H, dd), 7.99-7.93 (5H, m), 7.83-7.74 (3H, m), |
| | 7.59-7.38 (7H, m), 7.23-7.17 (4H, m) |
| 54 | δ = 8.55 (1H, dd), 8.22 (1H, dd), 8.00-7.82 (6H, m), 7.71 (1H, dd), |
| | 7.59-7.36 (8H, m), 7.23-7.19 (2H, m) |
| 56 | δ = 8.56 (1H, dd), 8.21 (1H, dd), 7.97-7.83 (6H, m), 7.70 (1H, dd), |
| | 7.60-7.36 (8H, m), 7.19-7.14 (2H, m) |
| 61 | δ = 8.57 (1H, dd), 8.38-8.33 (2H, m), 8.17 (1H, dd), 7.97-7.93 (3H, m), 7.78 |
| | (1H, td), 7.62-7.49 (6H, m), 7.37 (1H, td), 7.22-7.18 (2H, m) |
| 63 | δ = 8.54 (1H, dd), 8.37-8.30 (2H, m), 8.22 (1H, dd), 7.98-7.89 (3H, m), 7.75 |
| | (1H, td), 7.60-7.46 (6H, m), 7.33 (1H, td), 7.21-7.18 (2H, m) |
| 68 | δ = 8.56 (1H, dd), 8.23 (1H, dd), 8.01-7.84 (6H, m), 7.72 (1H, dd), |
| | 7.61-7.36 (8H, m), 7.23-7.18 (2H, m) |
| 70 | δ = 8.53 (1H, dd), 8.18 (1H, dd), 7.96-7.81 (5H, m), 7.76-7.70 |
| | (2H, dd), 7.65-7.38 (8H, m), 7.21-7.17 (2H, m) |
| 73 | δ = 8.36-8.31 (2H, m), 7.96-7.90 (3H, m), 7.82 (1H, dd), 7.69-7.50 |
| | (8H, m), 7.39-7.31 (2H, m) |
| 76 | δ = 8.03-7.96 (5H, m), 7.82-7.69 (4H, m), 7.60-7.28 (11H, m) |
| 77 | δ = 8.01-7.95 (4H, m), 7.82 (2H, dd), 7.69-7.54 (8H, m), 7.40-7.33 (4H, m) |
| 84 | δ = 8.54 (1H, dd), 8.43 (1H, dd), 7.98-7.91 (5H, m), 7.81 (1H, dd), 7.69-7.31 |
| | (10H, m) |
| 85 | δ = 8.33-8.28 (2H, m), 7.99-7.91 (3H, m), 7.79 (1H, dd), 7.66-7.49 |
| | (8H, m), 7.41-7.33 (2H, m) |
| 86 | δ = 7.99-7.91 (5H, m), 7.84-7.72 (4H, m), 7.58-7.25 (11H, m) |
| 93 | δ = 8.52 (1H, dd), 8.40 (1H, dd), 8.02-7.93 (5H, m), 7.84 |
| | (1H, dd), 7.72-7.36 (10H, m) |
| 98 | δ = 8.41-8.35 (2H, m), 7.99-7.93 (3H, m), 7.78 (1H, dd), 7.66-7.47 |
| | (8H, m), 7.37-7.29 (2H, m) |
| 104 | δ = 8.02-7.97 (4H, m), 7.77 (2H, dd), 7.71-7.53 (8H, m), 7.42-7.36 (4H, m) |
| 109 | δ = 8.34-8.29 (2H, m), 7.98-7.92 (3H, m), 7.85 (1H, dd), 7.71-7.52 |
| | (8H, m), 7.43-7.35 (2H, m) |
| 11 | δ = 8.05-7.93 (5H, m), 7.85-7.72 (4H, m), 7.63-7.33 (11H, m) |
| 112 | δ = 8.02-7.90 (5H, m), 7.82-7.69 (4H, m), 7.60-7.36 (11H, m) |
| 115 | δ = 8.03-7.93 (4H, m), 7.78 (2H, dd), 7.71-7.56 (8H, m), 7.42-7.35 (4H, m) |
| 116 | δ = 8.02-7.93 (4H, m), 7.81 (2H, dd), 7.68-7.52 (8H, m), 7.41-7.38 (4H, m) |
| 118 | δ = 8.56 (1H, dd), 8.41 (1H, dd), 8.03-7.95 (5H, m), 7.84 (1H, dd), |
| | 7.72-7.34 (10H, m) |
| 121 | δ = 8.37-8.32 (2H, m), 8.02-7.93 (3H, m), 7.80 (1H, dd), 7.68-7.46 |
| | (8H, m), 7.37-7.29 (2H, m) |
| 128 | δ = 8.03-7.96 (4H, m), 7.84 (2H, dd), 7.71-7.59 (8H, m), 7.37-7.31 (4H, m) |
| 130 | δ = 8.56 (1H, dd), 8.39 (1H, dd), 7.99-7.92 (5H, m), 7.77 (1H, dd), |
| | 7.72-7.33 (10H, m) |
| 133 | δ = 8.40-8.33 (2H, m), 7.99-7.94 (3H, m), 7.85 (1H, dd), 7.70-7.49 |
| | (8H, m), 7.38-7.33 (2H, m) |
| 137 | δ = 8.03-7.91 (4H, m), 7.83 (2H, dd), 7.67-7.52 (8H, m), 7.38-7.29 (4H, m) |
| 144 | δ = 8.56 (1H, dd), 8.41 (1H, dd), 8.00-7.92 (5H, m), 7.78 (1H, dd), 7.73-7.36 |
| | (10H, m) |
| 149 | δ = 8.03-7.92 (4H, m), 7.79 (2H, dd), 7.72-7.59 (8H, m), 7.45-7.38 (4H, m) |
| 156 | δ = 8.55 (1H, dd), 8.45 (1H, dd), 8.01-7.94 (5H, m), 7.76 (1H, dd), 7.70-7.36 |
| | (10H, m) |
| 157 | δ = 8.39-8.33 (2H, m), 7.99-7.93 (3H, m), 7.87 (1H, dd), 7.71-7.52 (8H, m), |
| | 7.43-7.34 (2H, m) |
| 159 | δ = 8.05-7.93 (5H, m), 7.81-7.70 (4H, m), 7.58-7.30 (11H, m) |
| 162 | δ = 8.04-7.94 (4H, m), 7.77 (2H, dd), 7.65-7.51 (8H, m), 7.44-7.35 (4H, m) |
| 169 | δ = 8.38-8.30 (2H, m), 7.95-7.88 (3H, m), 7.79 (1H, dd), 7.71-7.48 (8H, m), |
| | 7.41-7.32 (2H, m) |
| 173 | δ = 8.02-7.93 (4H, m), 7.80 (2H, dd), 7.71-7.58 (8H, m), 7.39-7.35 (4H, m) |
| 182 | δ = 8.06-7.97 (5H, m), 7.76-7.66 (4H, m), 7.57-7.30 (11H, m) |
| 189 | δ = 8.50 (1H, dd), 8.41 (1H, dd), 7.99-7.88 (5H, m), 7.79 (1H, dd), |
| | 7.65-7.34 (10H, m) |
| 194 | δ = 8.05-7.94 (5H, m), 7.80-7.70 (4H, m), 7.62-7.31 (11H, m) |
| 195 | δ = 8.52 (1H, dd), 8.41 (1H, dd), 8.01-7.92 (5H, m), 7.83 (1H, dd), |
| | 7.71-7.36 (10H, m) |
| 207 | δ = 8.03-7.93 (5H, m), 7.83-7.73 (4H, m), 7.63-7.30 (11H, m) |
| 213 | δ = 8.53 (1H, dd), 8.45 (1H, dd), 8.00-7.94 (5H, m), 7.78 (1H, dd), |
| | 7.73-7.32 (10H, m) |
| 217 | δ = 8.45 (1H, dd), 8.36-8.31 (2H, m), 8.03-7.94 (5H, m), 7.73-7.32 (8H, m) |
| 220 | δ = 8.43 (1H, dd), 8.04-7.92 (7H, m), 7.75-7.49 (10H, m), 7.32-7.28 (2H, m) |
| 221 | δ = 8.56 (1H, dd), 8.38-8.34 (2H, m), 7.99-7.90 (5H, m), 7.80-7.75 (4H, m), |
| | 7.50-7.41 (8H, m), 7.16 (1H, t) |
| 224 | δ = 8.53 (1H, dd), 8.40-8.35 (2H, m), 8.01-7.93 (5H, m), 7.78-7.72 (4H, m), |
| | 7.48-7.40 (8H, m), 7.21 (1H, t) |
| 226 | δ = 8.55 (1H, dd), 8.38-8.31 (2H, m), 8.00-7.91 (5H, m), 7.82-7.74 (4H, m), |
| | 7.46-7.38 (8H, m), 7.18 (1H, t) |
| 227 | δ = 8.48 (1H, dd), 8.32-8.25 (2H, m), 8.00-7.92 (5H, m), 7.76-7.37 (8H, m) |
| 233 | δ = 8.55 (1H, dd), 8.41 (1H, dd), 8.03-7.95 (5H, m), 7.74 (1H, dd), |
| | 7.70-7.36 (10H, m) |
| 235 | δ = 8.52 (1H, dd), 8.34-8.30 (2H, m), 7.98-7.80 (6H, m), 7.54-7.35 |
| | (9H, m), 7.16 (1H, t) |
| 236 | δ = 8.52 (1H, dd), 8.42 (1H, d), 8.34-8.30 (2H, m), 7.98-7.80 (6H, m), |
| | 7.54-7.35 (8H, m), 7.16 (1H, t) |
| 237 | δ = 8.52 (1H, dd), 8.35-8.32 (2H, m), 7.98-7.80 (8H, m), 7.79-7.65 |
| | (5H, m), 7.54-7.35 (6H, m), 7.16 (1H, t) |
| 239 | δ = 8.52 (1H, dd), 8.41 (1H, d), 8.36-8.31 (2H, m), 8.03-7.91 (5H, m), |
| | 7.61-7.46 (8H, m), 7.33 (1H, t), 7.16 (1H, t) |
| 242 | δ = 8.50 (1H, d), 8.41 (1H, d), 8.24-8.21 (2H, m), 8.13 (1H, d), |
| | 7.99-7.84 (8H, m), 7.68-7.31 (6H, m), 7.16-7.13 (2H, m) |
| 244 | δ = 8.52 (1H, d), 8.22-8.18 (2H, m), 7.98-7.93 (3H, m), 7.82 (1H, d), |
| | 7.69-7.51 (8H, m), 7.39-7.31 (5H, m), 7.13 (1H, t) |
| 251 | δ = 8.56-8.54 (2H, m), 8.38-8.35 (2H, m), 8.21-8.17 (2H, m), 8.11 (1H, d), |
| | 7.96-7.94 (2H, m), 7.68-7.51 (11H, m), 7.32-7.29 (2H, m), 7.18-7.16 (2H, m) |
| 254 | δ = 8.46 (1H, dd), 8.00-7.91 (7H, m), 7.78-7.55 (10H, m), 7.36-7.30 (2H, m) |
| 270 | δ = 8.54 (1H, dd), 8.39 (1H, dd), 7.96-7.89 (5H, m), 7.80 (1H, dd), |
| | 7.65-7.28 (10H, m) |
| 275 | δ = 8.47 (1H, dd), 8.40-8.36 (2H, m), 7.98-7.91 (5H, m), 7.75-7.36 (8H, m) |
| 282 | δ = 8.55 (1H, dd), 8.41 (1H, dd), 7.96-7.90 (5H, m), 7.78 (1H, dd), |
| | 7.70-7.37 (10H, m) |
| 289 | δ = 8.45 (1H, dd), 8.01-7.93 (7H, m), 7.79-7.52 (10H, m), 7.39-7.36 (2H, m) |
| 295 | δ = 8.46-8.42 (2H, m), 8.04-7.92 (8H, m), 7.70-7.68 (2H, m), 7.56-7.49 |
| | (4H, m), 7.32-7.28 (2H, m) |
| 301 | δ = 8.46 (1H, dd), 8.00-7.94 (7H, m), 7.80-7.53 (10H, m), 7.36-7.30 (2H, m) |
| 307 | δ = 8.49-8.45 (2H, m), 8.00-7.90 (8H, m), 7.73-7.69 (2H, m), 7.61-7.52 |
| | (4H, m), 7.33-7.29 (2H, m) |
| 312 | δ = 8.44 (1H, dd), 8.03-7.95 (7H, m), 7.78-7.50 (10H, m), 7.33-7.28 (2H, m) |
| 323 | δ = 8.51 (1H, dd), 8.43-8.39 (2H, m), 7.99-7.89 (5H, m), 7.78-7.41 (8H, m) |
| 331 | δ = 8.51-8.46 (2H, m), 8.00-7.91 (8H, m), 7.72-7.67 (2H, m), 7.55-7.46 |
| | (4H, m), 7.36-7.30 (2H, m) |
| 338 | δ = 8.55 (1H, dd), 8.38 (1H, dd), 8.00-7.94 (5H, m), 7.86 (1H, dd), |
| | 7.70-7.37 (10H, m) |
| 343 | δ = 8.51 (1H, dd), 8.35-8.27 (2H, m), 7.99-7.90 (5H, m), 7.73-7.40 (8H, m) |
| 351 | δ = 8.45-8.41 (2H, m), 8.02-7.94 (8H, m), 7.74-7.70 (2H, m), 7.59-7.47 |
| | (4H, m), 7.35-7.29 (2H, m) |
| 360 | δ = 8.51 (1H, s), 7.98-7.94 (4H, m), 7.91-7.79 (4H, m), 7.68-7.60 (5H, m), |
| | 7.58-7.54 (3H, m), 7.50 (2H, s), 7.39-7.35 (2H, m), 7.31 (2H, dd), |
| 361 | δ = 8.53 (1H, s), 8.03-7.96 (4H, m), 7.94-7.91 (2H, m), 7.82-7.76 (2H, m), |
| | 7.68-7.60 (5H, m), 7.58-7.50 (5H, m), 7.38-7.32 (2H, m), 7.31 (1H, dd), 7.16 |
| | (2H, dd), |
| 367 | δ = 8.55 (1H, s), 8.36 (2H, d), 7.99-7.94 (4H, m), 7.89 (1H, d), 7.77 |
| | (1H, d), 7.62-7.50 (10H, m), 7.37 (1H, d), 7.33 (1H, dd), |
| 371 | δ = 8.54 (1H, s), 7.99-7.94 (5H, m), 7.89-7.82 (2H, m), 7.77 (1H, d), 7.69-7.58 |
| | (6H, m), 7.57-7.50 (4H, m), 7.39-7.34 (2H, m), 7.31 (1H, dd), 7.18 (2H, dd), |
| 376 | δ = 8.55 (1H, s), 8.43 (1H, d), 8.12 (1H, d), 7.98-7.92 (7H, m), 7.89 (1H, d), |
| | 7.77 (1H, dd), 7.62-7.49 (9H, m), 7.33 (1H, t), 7.16 (2H, dd), |
| 381 | δ = 8.53 (1H, s), 8.38-8.31 (2H, m), 7.96-7.91 (5H, m), 7.75-7.73 (4H, m), |
| | 7.62-7.58 (6H, m), 7.50-7.47 (4H, m), 7.41-7.37 (2H, m), 7.18 (1H, dd), |
| 386 | δ = 8.54 (1H, s), 8.31 (1H, d), 8.08 (1H, d), 7.98-7.91 (5H, m), 7.88 (1H, d), |
| | 7.74 (1H, d), 7.62-7.56 (8H, m), 7.54-7.49 (5H, m), 7.41-7.35 (3H, dd), 7.31 |
| | (1H, t), 7.15 (1H, d), |
| 396 | δ = 8.52 (1H, s), 8.29 (1H, d), 8.06 (1H, d), 7.98-7.93 (4H, m), 7.88-7.79 (3H, |
| | m), 7.62-7.54 (9H, m), 7.50-7.45 (4H, m), 7.41-7.31 (4H, m), 7.17 (1H, d), |
| 406 | δ = 8.52 (1H, s), 8.38 (1H, d), 7.96-7.91 (5H, m), 7.75-7.68 (4H, m), 7.62-7.56 |
| | (5H, m), 7.49-7.41 (5H, m), 7.35-7.31 (3H, m), 7.28 (2H, dd), |
| 414 | δ = 8.53 (2H, s), 8.42 (1H, d), 8.36 (1H, d), 7.94-7.91 (5H, m), 7.73-7.68 (3H, |
| | m), 7.62-7.56 (8H, m), 7.48-7.41 (6H, m), 7.35 (1H, dd), 7.16 (1H, dd), |
| 415 | δ = 8.54 (1H, s), 8.35 (1H, d), 8.32 (2H, d), 7.97-7.89 (4H, m), 7.77-7.73 (2H, |
| | m), 7.62-7.58 (4H, m), 7.55 (3H, d), 7.48-7.40 (8H, m), 7.33 (1H, s), |
| | 7.15 (2H, t), |
| 420 | δ = 8.55 (1H, s), 8.34 (1H, d), 7.97-7.89 (5H, m), 7.88-7.73 (5H, m), 7.61-7.54 |
| | (8H, m), 7.50-7.39 (6H, m), 7.34 (1H, t), 7.30 (1H, d), 7.16 (1H, d), |
| 422 | δ =8.51 (1H, s), 8.37 (1H, d), 8.08 (1H, d), 7.96-7.92 (4H, m), 7.89-7.86 (2H, |
| | d), 7.77-7.61 (6H, m), 7.58-7.50 (6H, m), 7.49 (2H, t), 7.41-7.35 (3H, m), 7.33 |
| | (1H, d), 7.18 (2H, d), |
| 427 | δ = 8.53 (1H, s), 8.35 (1H, d), 8.33-8.30 (3H, m), 7.94-7.91 (3H, m), 7.74-7.71 |
| | (2H, d), 7.62-7.55 (6H, m), 7.50-7.45 (7H, m), 7.41 (1H, dd), 7.34 |
| | (1H, s), 7.31 (1H, t), |
| 432 | δ = 8.55 (1H, s), 8.38 (1H, d), 8.31 (1H, d), 7.98 (1H, d), 7.94 (2H, d), 7.91 (1H, d), |
| | 7.88 (1H, d), 7.83 (1H, d), 7.79 (1H, d), 7.74 (1H, dd), 7.73 (1H, d), 7.62 (3H, d), |
| | 7.61 (1H, dd), 7.58 (2H, dd), 7.54 (1H, s), 7.5 (2H, t), 7.49 (2H, t), 7.41 (1H, dd), |
| | 7.39 (1H, dd), 7.35 (1H, t), 7.31 (1H, d), 7.16 (2H, d), |
| 438 | δ = 8.55 (2H, s), 8.45 (1H, d), 8.38 (1H, d), 8.31 (1H, d), 7.94 (2H, d), 7.93 (1H, d), |
| | 7.92 (1H, d), 7.91 (1H, d), 7.74 (1H, d), 7.73 (1H, dd), 7.7 (1H, d), 7.62 (3H, d), |
| | 7.61 (1H, dd), 7.58 (2H, dd), 7.56 (1H, s), 7.5 (2H, t), 7.49 (3H, t), 7.41 (1H, dd), |
| | 7.35 (1H, dd), 7.16 (2H, t), |
| 439 | δ = 8.55 (1H, s), 8.38 (1H, d), 8.36 (2H, d), 8.29 (1H, d), 8.06 (1H, d), 7.94 (2H, d), |
| | 7.73 (1H, d), 7.62 (3H, d), 7.61 (1H, d), 7.58 (2H, dd), 7.5 (5H, d), 7.49 (2H, d), |
| | 7.48 (1H, dd), 7.41 (1H, dd), 7.35 (2H, s), |
| 451 | δ = 8.55 (1H, s), 8.36 (2H, d), 7.96 (2H, d), 7.94 (1H, d), 7.91 (1H, d), 7.68 (1H, d), |
| | 7.62 (3H, d), 7.58 (2H, d), 7.56 (1H, d), 7.5 (5H, dd), 7.49 (2H, d), 7.41 (1H, d), |
| | 7.35 (1H, dd), 7.25 (2H, dd), 7.16 (4H, s), |
| 455 | δ = 8.53 (1H, s), 7.97-7.91 (5H, m), 7.82 (1H, d), 7.69-7.62 (5H, m), 7.58-7.54 |
| | (5H, m), 7.48-7.41 (5H, m), 7.39-7.31 (3H, m), 7.22 (2H, t), 7.14 (2H, d), |
| 464 | δ = 8.54 (1H, s), 8.00 (1H, d), 7.96-7.89 (6H, m), 7.79-7.77 (3H, m), 7.62-7.58 |
| | (5H, m), 7.48-7.41 (5H, m), 7.35 (1H, dd), 7.28 (4H, s), |
| 470 | δ = 8.55 (1H, s), 8.06 (1H, d), 7.98-7.88 (7H, m), 7.77-7.60 (5H, m), 7.58-7.51 |
| | (4H, m), 7.49-7.43 (5H, m), 7.39-7.31 (3H, m), 7.23 (2H, d), 7.14 (1H, d), |
| 471 | δ = 8.54 (1H, s), 8.43 (1H, d), 8.03 (1H, d), 7.99-7.93 (6H, m), 7.89 (1H, d), |
| | 7.77 (1H, d), 7.68-7.61 (5H, m), 7.58-7.56 (3H, m), 7.48-7.44 (5H, m), |
| | 7.41-7.36 (2H, m), 7.21 (2H, t), 7.17 (1H, d), |
| 475 | δ = 8.54 (1H, s), 8.36-8.31 (3H, m), 7.96-7.91 (4H, m), 7.74 (1H, d), 7.62-7.58 |
| | (6H, m), 7.49-7.40 (8H, m), 7.33-7.27 (4H, m), |
| 476 | δ = 8.56 (1H, s), 8.31 (1H, d), 7.95-7.91 (6H, m), 7.79-7.74 (3H, m), 7.62-7.58 |
| | (5H, m), 7.48-7.43 (5H, m), 7.37-7.28 (5H, m) |
| 479 | δ = 8.55 (1H, s), 8.31 (1H, d), 7.98-7.91 (5H, m), 7.82 (1H, d), 7.72 (1H, d), |
| | 7.67-7.61 (4H, m), 7.58-7.51 (6H, m), 7.46-7.39 (4H, m), 7.35-7.31 (2H, m), |
| | 7.22 (2H, d), 7.18 (1H, d), |
| 503 | δ = 8.83-8.76 (2H, m), 8.55 (1H, d), 8.19 (1H, d), 7.99-7.91 (5H, m), 7.82-7.79 |
| | (2H, m), 7.69-7.57 (6H, m), 7.54-7.45 (4H, m), 7.41-7.33 (4H, m) |
| 506 | δ = 8.55 (1H, s), 8.19 (1H, d), 8.08-7.98 (4H, m), 7.95-7.87 (5H, m), 7.80 (1H, |
| | d), 7.65-7.55 (5H, m), 7.51-7.43 (5H, m), 7.38-7.32 (3H, m), 7.20 (1H, d) |
| 510 | δ = 8.52 (2H, s), 8.45 (1H, d), 8.19 (1H, d), 8.05 (2H, d), 7.95-7.91 (6H, m), |
| | 7.79-7.75 (2H, m), 7.65-7.62 (2H, m), 7.58-7.50 (4H, m), 7.49-7.42 (4H, m), |
| | 7.35 (1H, t), 7.22-7.15 (4H, m) |
| 511 | δ = 8.95 (1H, s), 8.55 (1H, d), 8.36 (2H, d), 8.29 (1H, d), 8.19 (1H, d), |
| | 7.95-7.89 (5H, m), 7.83 (1H, d), 7.71 (2H, d), 7.58-7.49 (6H, m), 7.46-7.37 (3H, m), |
| | 7.23-7.20 (2H, m), |
| 512 | δ = 9.09 (1H, s), 8.53 (1H, d), 8.37 (1H, d), 8.19 (1H, d), 8.12 (1H, d), |
| | 7.96-7.88 (5H, m), 7.79-7.73 (4H, m), 7.65-7.59 (3H, m), 7.49-7.40 (5H, m), 7.35 |
| | (1H, t), 7.19-7.15 (6H, m) |
| 513 | δ = 9.07 (1H, s), 8.50 (1H, d), 8.17-8.14 (2H, m), 7.98-7.89 (5H, m), |
| | 7.78-7.69 (5H, m), 7.65 (1H, d), 7.58-7.47 (8H, m), 7.41-7.36 (3H, m) |
| 516 | δ = 8.93 (1H, s), 8.51 (1H, d), 8.29 (1H, d), 8.16 (1H, d), 8.09 (1H, d), |
| | 7.98-7.88 (7H, m), 7.77-7.71 (3H, m), 7.58-7.51 (4H, m), 7.49-7.42 (3H, m), |
| | 7.38-7.32 (3H, m) |
| 518 | δ = 8.96 (1H, s), 8.52 (1H, d), 8.47 (1H, d), 8.27 (1H, d), 8.21 (1H, d), 8.12 |
| | (1H, d), 7.99-7.87 (8H, m), 7.78-7.73 (3H, m), 7.58-7.51 (3H, m), 7.47-7.38 |
| | (5H, m), 7.18-7.13 (4H, m) |

TABLE 3

| No. | FD-MS | No. | FD-MS |
|---|---|---|---|
| 1 | m/z = 635.30(C₄₅H₁₇D₁₁N₄ = 635.81) | 4 | m/z = 711.33(C₅₁H₂₁D₁₁N₄ = 711.91) |
| 5 | m/z = 725.31(C₅₁H₁₉D₁₁N₄O = 725.90) | 7 | m/z = 725.31(C₅₁H₁₉D₁₁N₄O = 725.90) |
| 10 | m/z = 741.29(C₅₁H₁₉D₁₁N₄S = 741.96) | 12 | m/z = 741.29(C₅₁H₁₉D₁₁N₄S = 741.96) |
| 14 | m/z = 711.33(C₅₁H₂₁D₁₁N₄ = 711.91) | 15 | m/z = 711.33(C₅₁H₂₁D₁₁N₄ = 711.91) |
| 18 | m/z = 725.31(C₅₁H₁₉D₁₁N₄O = 725.90) | 20 | m/z = 725.31(C₅₁H₁₉D₁₁N₄O = 725.90) |
| 21 | m/z = 741.29(C₅₁H₁₉D₁₁N₄S = 741.96) | 23 | m/z = 741.29(C₅₁H₁₉D₁₁N₄S = 741.96) |
| 25 | m/z = 635.30(C₄₅H₁₇D₁₁N₄ = 635.81) | 29 | m/z = 725.31(C₅₁H₁₉D₁₁N₄O = 725.90) |
| 37 | m/z = 635.30(C₄₅H₁₇D₁₁N₄ = 635.81) | 39 | m/z = 711.33(C₅₁H₂₁D₁₁N₄ = 711.91) |
| 41 | m/z = 725.31(C₅₁H₁₉D₁₁N₄O = 725.90) | 42 | m/z = 725.31(C₅₁H₁₉D₁₁N₄O = 725.90) |
| 43 | m/z = 725.31(C₅₁H₁₉D₁₁N₄O = 725.90) | 44 | m/z = 725.31(C₅₁H₁₉D₁₁N₄O = 725.90) |
| 46 | m/z = 741.29(C₅₁H₁₉D₁₁N₄S = 741.96) | 49 | m/z = 635.30(C₄₅H₁₇D₁₁N₄ = 635.81) |
| 50 | m/z = 711.33(C₅₁H₂₁D₁₁N₄ = 711.91) | 54 | m/z = 725.31(C₅₁H₁₉D₁₁N₄O = 725.90) |
| 56 | m/z = 725.31(C₅₁H₁₉D₁₁N₄O = 725.90) | 61 | m/z = 635.30(C₄₅H₁₇D₁₁N₄ = 635.81) |
| 63 | m/z = 711.33(C₅₁H₂₁D₁₁N₄ = 711.91) | 68 | m/z = 725.31(C₅₁H₁₉D₁₁N₄O = 725.90) |
| 70 | m/z = 741.29(C₅₁H₁₉D₁₁N₄S = 741.96) | 73 | m/z = 636.28(C₄₅H₁₆D₁₁N₃O = 636.80) |
| 76 | m/z = 712.32(C₅₁H₂₀D₁₁N₃O = 712.90) | 77 | m/z = 726.30(C₅₁H₁₈D₁₁N₃O₂ = 726.88) |
| 84 | m/z = 742.27(C₅₁H₁₈D₁₁N₃OS = 742.94) | 85 | m/z = 636.28(C₄₅H₁₆D₁₁N₃O = 636.80) |
| 86 | m/z = 712.32(C₅₁H₂₀D₁₁N₃O = 712.90) | 93 | m/z = 742.27(C₅₁H₁₈D₁₁N₃OS = 742.94) |
| 98 | m/z = 636.28(C₄₅H₁₆D₁₁N₃O = 636.80) | 104 | m/z = 726.30(C₅₁H₁₈D₁₁N₃O₂ = 726.88) |
| 109 | m/z = 636.28(C₄₅H₁₆D₁₁N₃O = 636.80) | 111 | m/z = 712.32(C₅₁H₂₀D₁₁N₃O = 712.90) |
| 112 | m/z = 712.32(C₅₁H₂₀D₁₁N₃O = 712.90) | 115 | m/z = 726.30(C₅₁H₁₈D₁₁N₃O₂ = 726.88) |
| 116 | m/z = 726.30(C₅₁H₁₈D₁₁N₃O₂ = 726.88) | 118 | m/z = 742.27(C₅₁H₁₈D₁₁N₃OS = 742.94) |
| 121 | m/z = 636.28(C₄₅H₁₆D₁₁N₃O = 636.80) | 128 | m/z = 726.30(C₅₁H₁₈D₁₁N₃O₂ = 726.88) |
| 130 | m/z = 742.27(C₅₁H₁₈D₁₁N₃OS = 742.94) | 133 | m/z = 636.28(C₄₅H₁₆D₁₁N₃O = 636.80) |
| 137 | m/z = 726.30(C₅₁H₁₈D₁₁N₃O₂ = 726.88) | 144 | m/z = 742.27(C₅₁H₁₈D₁₁N₃OS = 742.94) |
| 149 | m/z = 726.30(C₅₁H₁₈D₁₁N₃O₂ = 726.88) | 156 | m/z = 742.27(C₅₁H₁₈D₁₁N₃OS = 742.94) |
| 157 | m/z = 636.28(C₄₅H₁₆D₁₁N₃O = 636.80) | 159 | m/z = 712.32(C₅₁H₂₀D₁₁N₃O = 712.90) |
| 162 | m/z = 726.30(C₅₁H₁₈D₁₁N₃O₂ = 726.88) | 169 | m/z = 636.28(C₄₅H₁₆D₁₁N₃O = 636.80) |
| 173 | m/z = 726.30(C₅₁H₁₈D₁₁N₃O₂ = 726.88) | 182 | m/z = 712.32(C₅₁H₂₀D₁₁N₃O = 712.90) |
| 189 | m/z = 742.27(C₅₁H₁₈D₁₁N₃OS = 742.94) | 194 | m/z = 712.32(C₅₁H₂₀D₁₁N₃O = 712.90) |
| 195 | m/z = 742.27(C₅₁H₁₈D₁₁N₃OS = 742.94) | 207 | m/z = 712.32(C₅₁H₂₀D₁₁N₃O = 712.90) |
| 213 | m/z = 742.27(C₅₁H₁₈D₁₁N₃OS = 742.94) | 217 | m/z = 652.26(C₄₅H₁₆D₁₁N₃S = 652.86) |
| 220 | m/z = 728.29(C₅₁H₂₀D₁₁N₃S = 728.96) | 221 | m/z = 711.33(C₅₁H₂₁D₁₁N₄ = 711.91) |
| 224 | m/z = 711.33(C₅₁H₂₁D₁₁N₄ = 711.91) | 226 | m/z = 711.33(C₅₁H₂₁D₁₁N₄ = 711.91) |
| 227 | m/z = 652.26(C₄₅H₁₆D₁₁N₃S = 652.86) | 233 | m/z = 742.27(C₅₁H₁₈D₁₁N₃OS = 742.94) |
| 235 | m/z = 725.31(C₅₁H₁₉D₁₁N₄O = 725.90) | 236 | m/z = 741.29(C₅₁H₁₉D₁₁N₄S = 741.96) |
| 237 | m/z = 801.34(C₅₇H₂₃D₁₁N₄O = 801.99) | 239 | m/z = 725.31(C₅₁H₁₉D₁₁N₄O = 725.90) |
| 242 | m/z = 831.30(C₅₇H₂₁D₁₁N₄OS = 832.04) | 244 | m/z = 815.32(C₅₇H₂₁D₁₁N₄O₂ = 815.98) |
| 251 | m/z = 800.36(C₅₇H₂₄D₁₁N₅ = 801.01) | 254 | m/z = 728.29(C₅₁H₂₀D₁₁N₃S = 728.96) |
| 270 | m/z = 742.27(C₅₁H₁₈D₁₁N₃OS = 742.94) | 275 | m/z = 652.26(C₄₅H₁₆D₁₁N₃S = 652.86) |
| 282 | m/z = 742.27(C₅₁H₁₈D₁₁N₃OS = 742.94) | 289 | m/z = 728.29(C₅₁H₂₀D₁₁N₃S = 728.96) |
| 295 | m/z = 758.25(C₅₁H₁₈D₁₁N₃S₂ = 759.00) | 301 | m/z = 728.29(C₅₁H₂₀D₁₁N₃S = 728.96) |
| 307 | m/z = 758.25(C₅₁H₁₈D₁₁N₃S₂ = 759.00) | 312 | m/z = 728.29(C₅₁H₂₀D₁₁N₃S = 728.96) |
| 323 | m/z = 652.26(C₄₅H₁₆D₁₁N₃S = 652.86) | 331 | m/z = 758.25(C₅₁H₁₈D₁₁N₃S₂ = 759.00) |
| 338 | m/z = 742.27(C₅₁H₁₈D₁₁N₃OS = 742.94) | 343 | m/z = 652.26(C₄₅H₁₆D₁₁N₃S = 652.86) |
| 351 | m/z = 758.25(C₅₁H₁₈D₁₁N₃S₂ = 759.00) | 360 | m/z = 801.34(C₅₇H₂₃D₁₁N₄O = 801.99) |
| 361 | m/z = 801.34(C₅₇H₂₃D₁₁N₄O = 801.99) | 367 | m/z = 711.33(C₅₁H₂₁D₁₁N₄ = 711.91) |
| 371 | m/z = 801.34(C₅₇H₂₃D₁₁N₄O = 801.99) | 376 | m/z = 817.32(C₅₇H₂₃D₁₁N₄S = 818.05) |
| 381 | m/z = 787.36(C₅₇H₂₅D₁₁N₄ = 788.01) | 386 | m/z = 801.34(C₅₇H₂₃D₁₁N₄O = 801.99) |
| 396 | m/z = 801.34(C₅₇H₂₃D₁₁N₄O = 801.99) | 406 | m/z = 787.36(C₅₇H₂₅D₁₁N₄ = 788.01) |
| 414 | m/z = 817.32(C₅₇H₂₃D₁₁N₄S = 818.05) | 415 | m/z = 711.33(C₅₁H₂₁D₁₁N₄ = 711.91) |
| 420 | m/z = 801.34(C₅₇H₂₃D₁₁N₄O = 801.99) | 422 | m/z = 801.34(C₅₇H₂₃D₁₁N₄O = 801.99) |
| 427 | m/z = 711.33(C₅₁H₂₁D₁₁N₄ = 711.91) | 432 | m/z = 801.34(C₅₇H₂₃D₁₁N₄O = 801.99) |
| 438 | m/z = 817.32(C₅₇H₂₃D₁₁N₄S = 818.05) | 439 | m/z = 711.33(C₅₁H₂₁D₁₁N₄ = 711.91) |
| 451 | m/z = 711.33(C₅₁H₂₁D₁₁N₄ = 711.91) | 455 | m/z = 801.34(C₅₇H₂₃D₁₁N₄O = 801.99) |
| 464 | m/z = 787.36(C₅₇H₂₅D₁₁N₄ = 788.01) | 470 | m/z = 801.34(C₅₇H₂₃D₁₁N₄O = 801.99) |
| 471 | m/z = 817.32(C₅₇H₂₃D₁₁N₄S = 818.05) | 475 | m/z = 711.33(C₅₁H₂₁D₁₁N₄ = 711.91) |
| 476 | m/z = 787.36(C₅₇H₂₅D₁₁N₄ = 788.01) | 479 | m/z = 801.34(C₅₇H₂₃D₁₁N₄O = 801.99) |
| 503 | m/z = 721.29(C₅₁H₂₃D₇N₄O = 721.87) | 506 | m/z = 721.29(C₅₁H₂₃D₇N₄O = 721.87) |
| 510 | m/z = 737.26(C₅₁H₂₃D₇N₄S = 737.93) | 511 | m/z = 629.26(C₄₅H₂₃D₅N₄ = 629.78) |
| 512 | m/z = 707.31(C₅₁H₂₅D₇N₄ = 707.89) | 513 | m/z = 721.29(C₅₁H₂₃D₇N₄O = 721.87) |
| 516 | m/z = 719.27(C₅₁H₂₅D₅N₄O = 719.86) | 518 | m/z = 735.25(C₅₁H₂₅D₅N₄S = 735.92) |

### [Preparation Example 2] Preparation of Compound 2-1 and the Like

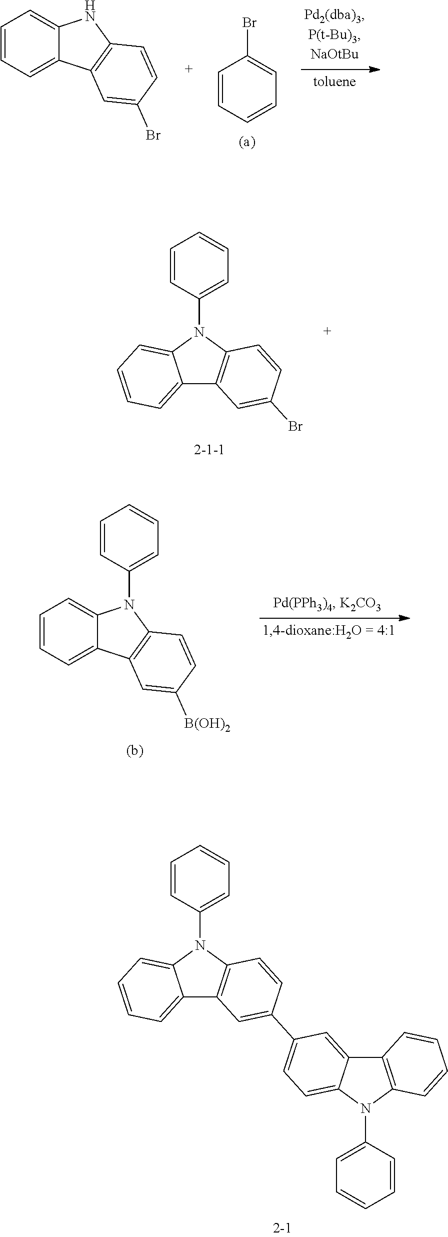 [SMILES: Brc1ccc2[nH]c3ccccc3c2c1 ; Brc1ccccc1 ; Brc1ccc2c(c1)c1ccccc1n2-c1ccccc1 ; OB(O)c1ccc2c(c1)c1ccccc1n2-c1ccccc1 ; c1ccc(-n2c3ccccc3c3cc(-c4ccc5c(c4)c4ccccc4n5-c4ccccc4)ccc32)cc1]

### 1) Preparation of Compound 2-1-1

After 10 g (49.59 mmol) of 3-bromo-9H-carbazole, 24.2 g (148.77 mmol) of 2-bromobenzene-1-ylium (a), 2.27 g (2.48 mmol) of tris(dibenzylideneacetone)dipalladium (Pd₂(dba)₃), 2.42 mL (9.92 mmol) of tri-tert-butylphosphine (P(t-Bu)₃), and 9.53 g (99.18 mmol) of sodium tert-butoxide (NatOBu) were put into a reaction flask, 100 mL of toluene was added thereto, and the resulting mixture was heated at 135° C. for 15 hours. When the reaction was terminated, the resulting product was extracted with methylene chloride (M C) and water, and then purified by column chromatography to obtain 14 g of Compound 2-1-1 (yield 98%).

### 2) Preparation of Compound 2-1

14 g (43.4 mmol) of Compound 2-1-1, 14.9 g (52 mmol) of (9-phenyl-9H-carbazol-3-yl)boronic acid (b), 2.5 g (2.17 mmol) of tetrakis(triphenylphosphine)palladium(0) (Pd(PPh₃)₄), and 17.9 g (130 mmol) of potassium carbonate (K₂CO₃) were put into a reaction flask, and then added to 140 mL of 1,4-dioxane and 35 mL of distilled water, and the resulting mixture was stirred at 120° C. for 4 hours.

Thereafter, a solid produced by lowering the temperature to room temperature was washed with distilled water and methanol to obtain 17 g (yield 80%) of Compound 2-1.

The target compounds in the following Table 4 were synthesized in the same manner as in the preparation of Preparation Example 2, except that Compound a in the following Table 4 was used instead of 2-bromobenzene-1-ylium (a), and Compound b in the following Table 4 was used instead of (9-phenyl-9H-carbozol-3-yl)boronic acid (b) in Preparation Example 2.

TABLE 4

| Compound | | | Target Compound |
|---|---|---|---|
| No. | Compound a | Compound b | (Yield %) |
| 2-2 | 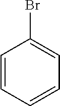 | 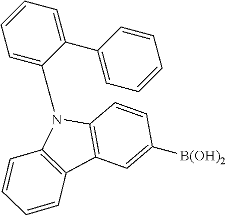 [SMILES: OB(O)c1ccc2c(c1)c1ccccc1n2-c1ccccc1-c1ccccc1] | 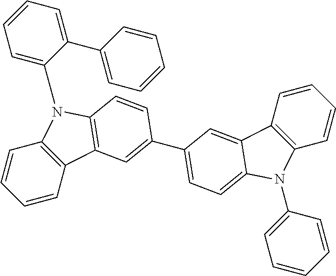 [SMILES: c1ccc(-c2ccccc2-n2c3ccccc3c3cc(-c4ccc5c(c4)c4ccccc4n5-c4ccccc4)ccc32)cc1] 82% |
| 2-3 | 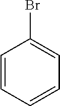 | 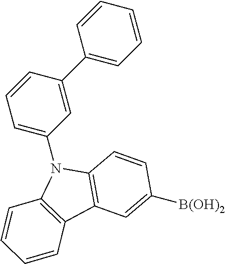 [SMILES: OB(O)c1ccc2c(c1)c1ccccc1n2-c1cccc(-c2ccccc2)c1] | 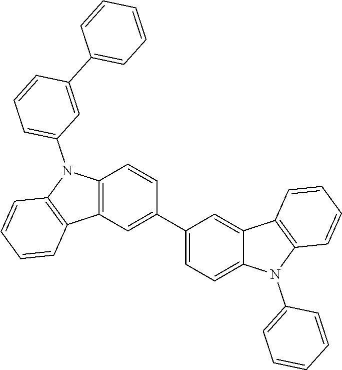 [SMILES: c1ccc(-c2cccc(-n3c4ccccc4c4cc(-c5ccc6c(c5)c5ccccc5n6-c5ccccc5)ccc43)c2)cc1] 80% |
| 2-4 | 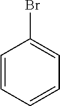 | 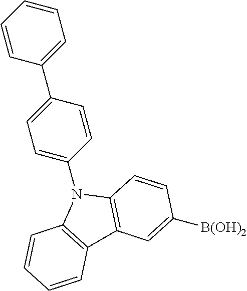 [SMILES: OB(O)c1ccc2c(c1)c1ccccc1n2-c1ccc(-c2ccccc2)cc1] | 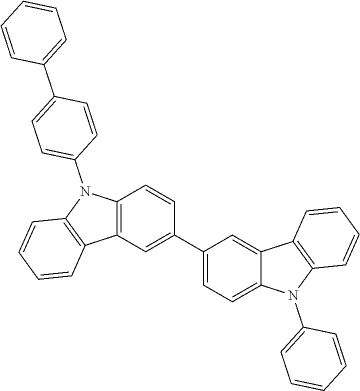 [SMILES: c1ccc(-c2ccc(-n3c4ccccc4c4cc(-c5ccc6c(c5)c5ccccc5n6-c5ccccc5)ccc43)cc2)cc1] 83% |
| 2-5 | 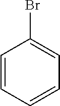 | 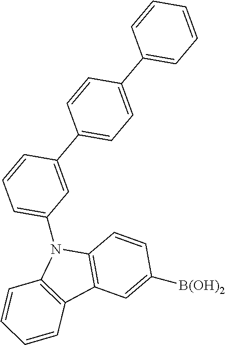 [SMILES: OB(O)c1ccc2c(c1)c1ccccc1n2-c1cccc(-c2ccc(-c3ccccc3)cc2)c1] | 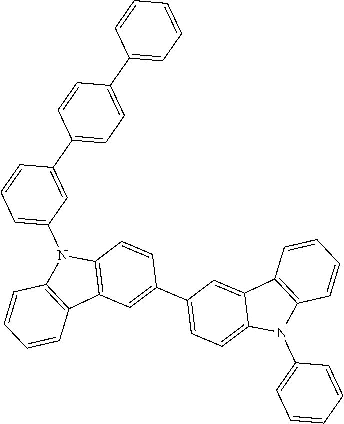 [SMILES: c1ccc(-c2ccc(-c3cccc(-n4c5ccccc5c5cc(-c6ccc7c(c6)c6ccccc6n7-c6ccccc6)ccc54)c3)cc2)cc1] 88% |
| 2-6 | 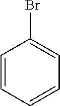 | 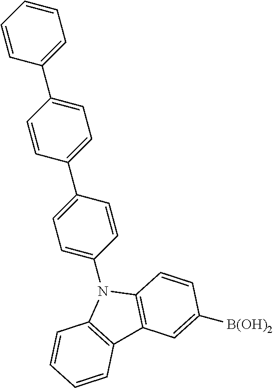 [SMILES: OB(O)c1ccc2c(c1)c1ccccc1n2-c1ccc(-c2ccc(-c3ccccc3)cc2)cc1] | 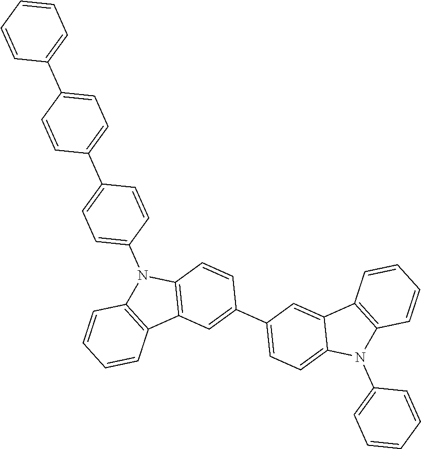 [SMILES: c1ccc(-c2ccc(-c3ccc(-n4c5ccccc5c5cc(-c6ccc7c(c6)c6ccccc6n7-c6ccccc6)ccc54)cc3)cc2)cc1] 86% |
| 2-7 | 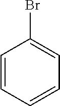 | 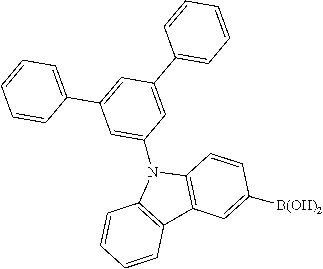 [SMILES: OB(O)c1ccc2c(c1)c1ccccc1n2-c1cc(-c2ccccc2)cc(-c2ccccc2)c1] | 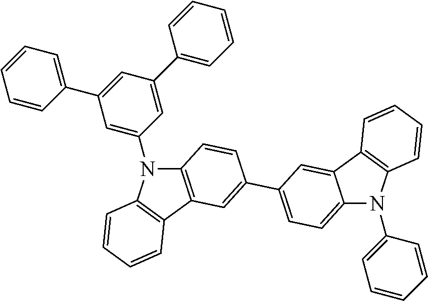 [SMILES: c1ccc(-c2cc(-c3ccccc3)cc(-n3c4ccccc4c4cc(-c5ccc6c(c5)c5ccccc5n6-c5ccccc5)ccc43)c2)cc1] 80% |
| 2-11 | 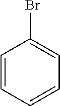 | 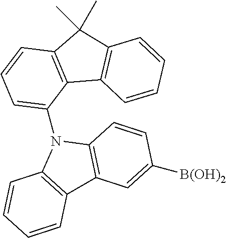 [SMILES: CC1(C)c2ccccc2-c2c(-n3c4ccccc4c4cc(B(O)O)ccc43)cccc21] | 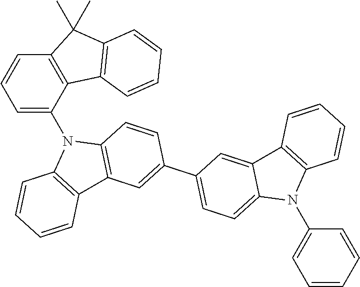 [SMILES: CC1(C)c2ccccc2-c2c(-n3c4ccccc4c4cc(-c5ccc6c(c5)c5ccccc5n6-c5ccccc5)ccc43)cccc21] 81% |
| 2-16 | 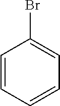 | 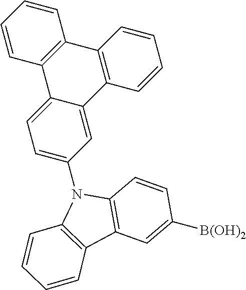 [SMILES: OB(O)c1ccc2c(c1)c1ccccc1n2-c1ccc2c3ccccc3c3ccccc3c2c1] | 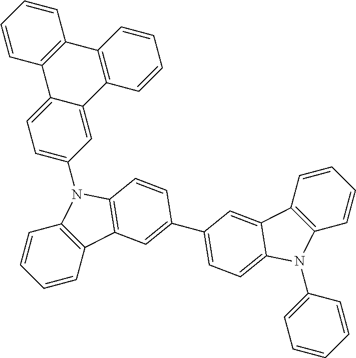 [SMILES: c1ccc(-n2c3ccccc3c3cc(-c4ccc5c(c4)c4ccccc4n5-c4ccc5c6ccccc6c6ccccc6c5c4)ccc32)cc1] 80% |
| 2-19 | 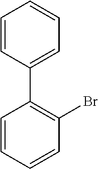 [SMILES: Brc1ccccc1-c1ccccc1] | 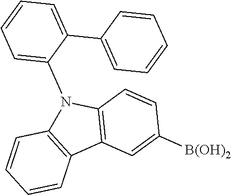 [SMILES: OB(O)c1ccc2c(c1)c1ccccc1n2-c1ccccc1-c1ccccc1] | 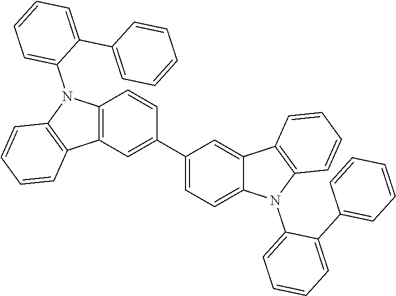 [SMILES: c1ccc(-c2ccccc2-n2c3ccccc3c3cc(-c4ccc5c(c4)c4ccccc4n5-c4ccccc4-c4ccccc4)ccc32)cc1] 83% |
| 2-20 | 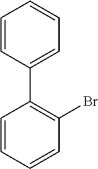 [SMILES: Brc1ccccc1-c1ccccc1] | 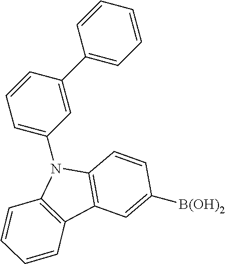 [SMILES: OB(O)c1ccc2c(c1)c1ccccc1n2-c1cccc(-c2ccccc2)c1] | 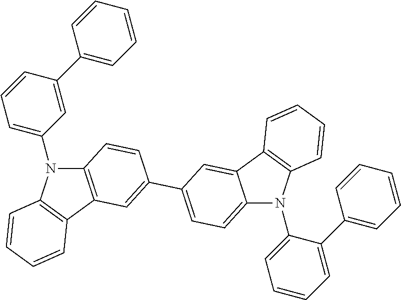 [SMILES: c1ccc(-c2cccc(-n3c4ccccc4c4cc(-c5ccc6c(c5)c5ccccc5n6-c5ccccc5-c5ccccc5)ccc43)c2)cc1] 81% |
| 2-21 | 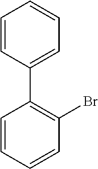 [SMILES: Brc1ccccc1-c1ccccc1] | 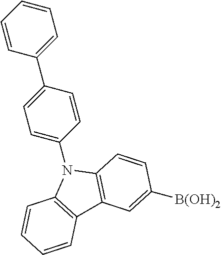 [SMILES: OB(O)c1ccc2c(c1)c1ccccc1n2-c1ccc(-c2ccccc2)cc1] | 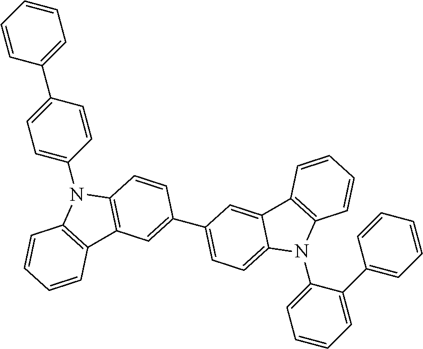 [SMILES: c1ccc(-c2ccc(-n3c4ccccc4c4cc(-c5ccc6c(c5)c5ccccc5n6-c5ccccc5-c5ccccc5)ccc43)cc2)cc1] 80% |
| 2-22 | 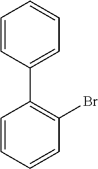 [SMILES: Brc1ccccc1-c1ccccc1] | 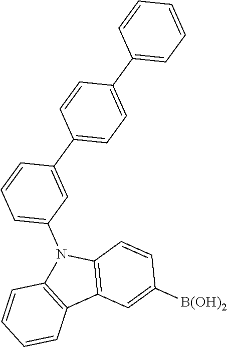 [SMILES: OB(O)c1ccc2c(c1)c1ccccc1n2-c1cccc(-c2ccc(-c3ccccc3)cc2)c1] | 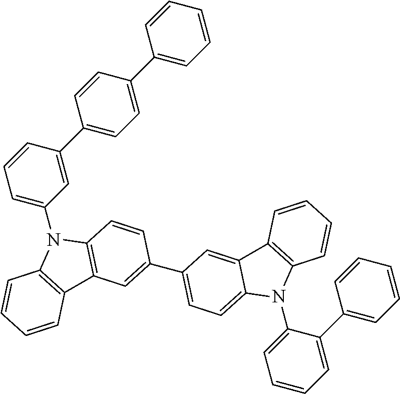 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1] 79% |
| 2-23 | 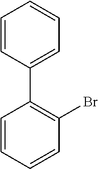 [SMILES: Brc1ccccc1-c1ccccc1] | 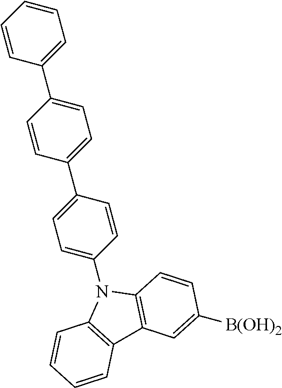 [SMILES: OB(O)c1ccc2c(c1)c1ccccc1n2-c1ccc(-c2ccc(-c3ccccc3)cc2)cc1] | 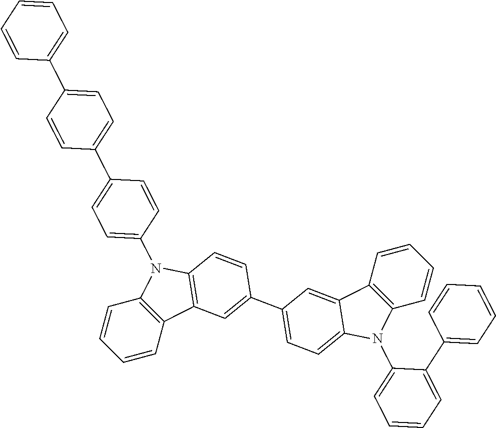 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] 82% |
| 2-26 | 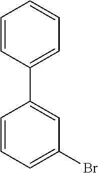 [SMILES: Brc1cccc(-c2ccccc2)c1] | 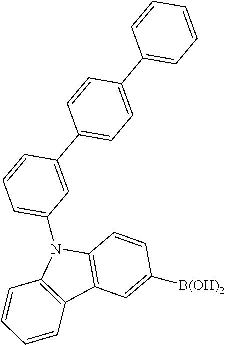 [SMILES: OB(O)c1ccc2c(c1)c1ccccc1n2-c1cccc(-c2ccc(-c3ccccc3)cc2)c1] | 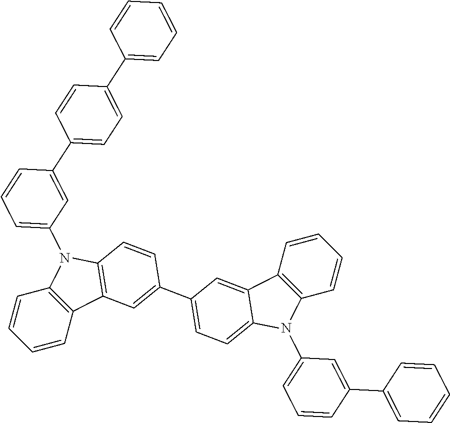 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1] 81% |
| 2-27 | 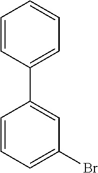 [SMILES: Brc1cccc(-c2ccccc2)c1] | 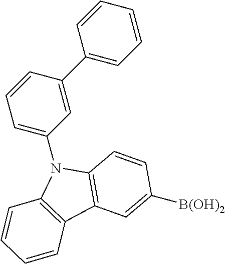 [SMILES: OB(O)c1ccc2c(c1)c1ccccc1n2-c1cccc(-c2ccccc2)c1] | 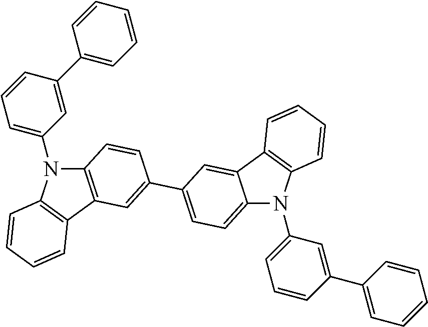 [SMILES: c1ccc(-c2cccc(-n3c4ccccc4c4cc(-c5ccc6c(c5)c5ccccc5n6-c5cccc(-c6ccccc6)c5)ccc43)c2)cc1] 85% |
| 2-28 | 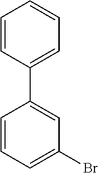 [SMILES: Brc1cccc(-c2ccccc2)c1] | 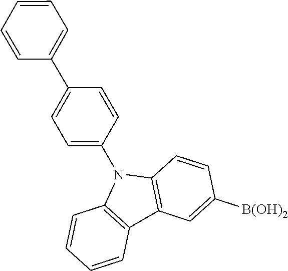 [SMILES: OB(O)c1ccc2c(c1)c1ccccc1n2-c1ccc(-c2ccccc2)cc1] | 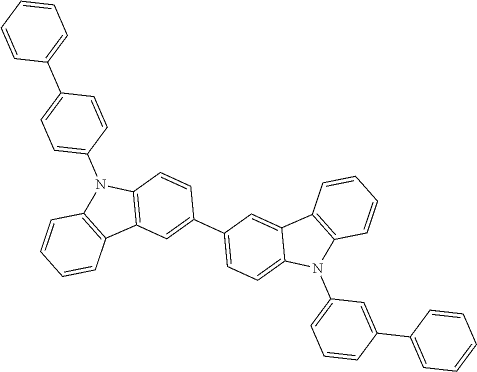 [SMILES: c1ccc(-c2ccc(-n3c4ccccc4c4cc(-c5ccc6c(c5)c5ccccc5n6-c5cccc(-c6ccccc6)c5)ccc43)cc2)cc1] 81% |
| 2-29 | 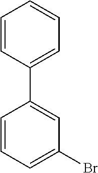 [SMILES: Brc1cccc(-c2ccccc2)c1] | 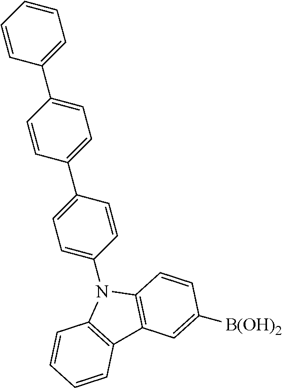 [SMILES: OB(O)c1ccc2c(c1)c1ccccc1n2-c1ccc(-c2ccc(-c3ccccc3)cc2)cc1] | 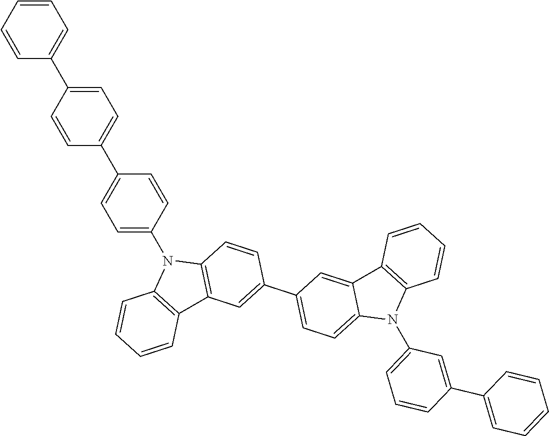 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] 80% |
| 2-30 | 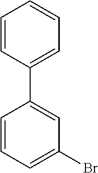 [SMILES: Brc1cccc(-c2ccccc2)c1] | 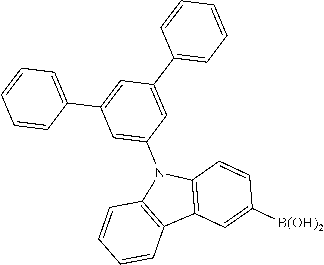 [SMILES: OB(O)c1ccc2c(c1)c1ccccc1n2-c1cc(-c2ccccc2)cc(-c2ccccc2)c1] | 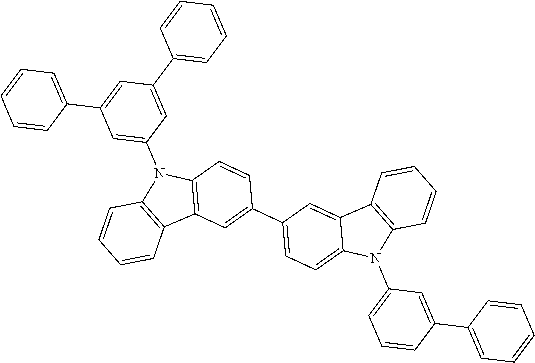 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] 79% |
| 2-32 | 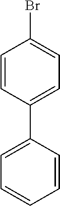 [SMILES: Brc1ccc(-c2ccccc2)cc1] | 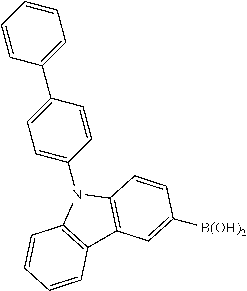 [SMILES: OB(O)c1ccc2c(c1)c1ccccc1n2-c1ccc(-c2ccccc2)cc1] | 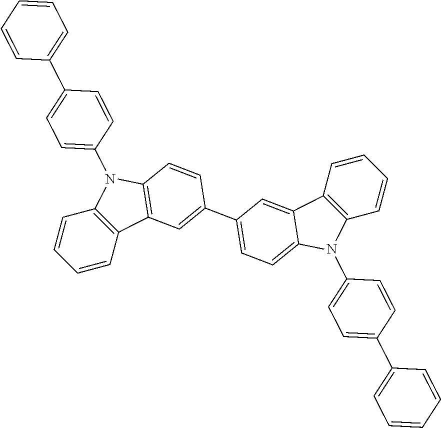 [SMILES: c1ccc(-c2ccc(-n3c4ccccc4c4cc(-c5ccc6c(c5)c5ccccc5n6-c5ccc(-c6ccccc6)cc5)ccc43)cc2)cc1] 81% |
| 2-33 | 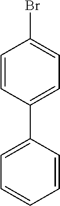 [SMILES: Brc1ccc(-c2ccccc2)cc1] | 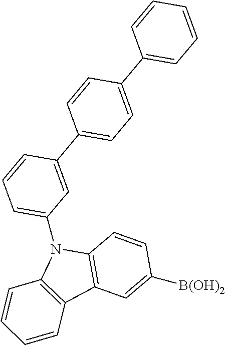 [SMILES: OB(O)c1ccc2c(c1)c1ccccc1n2-c1cccc(-c2ccc(-c3ccccc3)cc2)c1] | 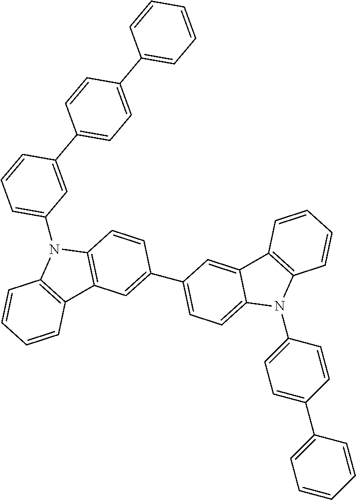 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1] 81% |
| 2-34 | 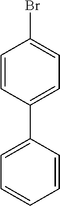 [SMILES: Brc1ccc(-c2ccccc2)cc1] | 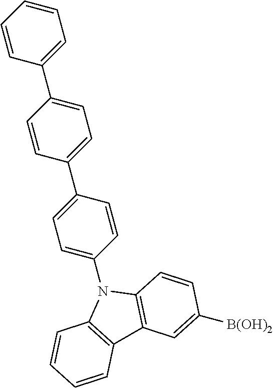 [SMILES: OB(O)c1ccc2c(c1)c1ccccc1n2-c1ccc(-c2ccc(-c3ccccc3)cc2)cc1] | 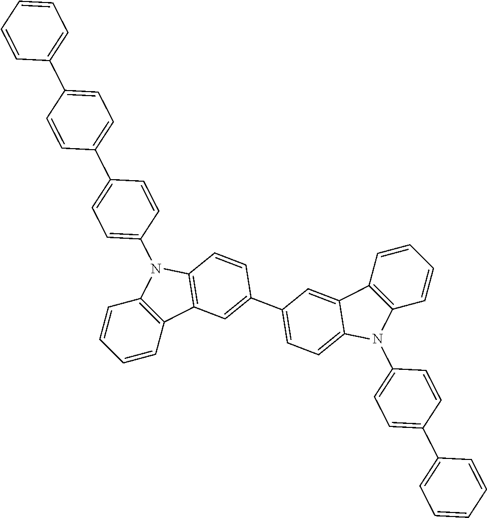 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1] 82% |
| 2-38 | 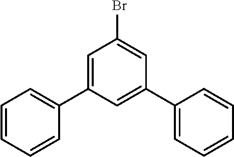 [SMILES: Brc1cc(-c2ccccc2)cc(-c2ccccc2)c1] | 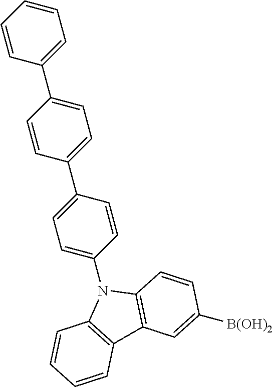 [SMILES: OB(O)c1ccc2c(c1)c1ccccc1n2-c1ccc(-c2ccc(-c3ccccc3)cc2)cc1] | 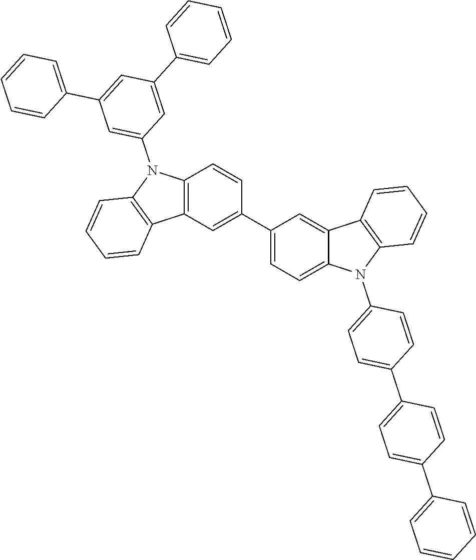 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1] 82% |
| 2-40 | 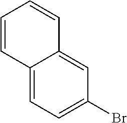 [SMILES: Brc1ccc2ccccc2c1] | 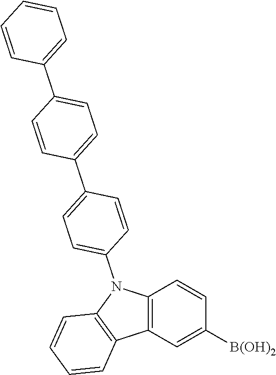 [SMILES: OB(O)c1ccc2c(c1)c1ccccc1n2-c1ccc(-c2ccc(-c3ccccc3)cc2)cc1] | 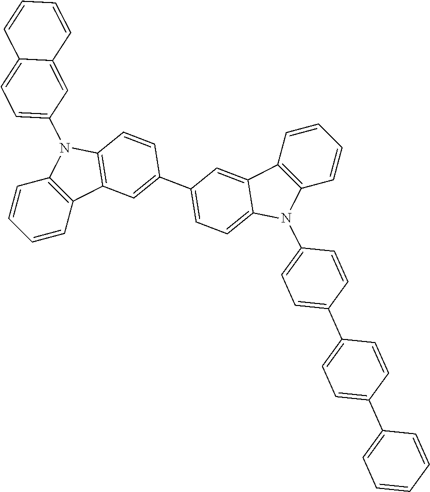 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1] 81% |
| 2-53 | 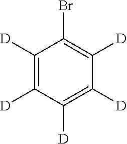 [SMILES: [2H]c1c([2H])c([2H])c(Br)c([2H])c1[2H]] | 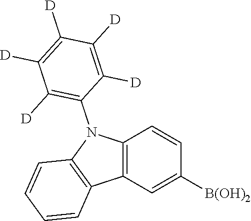 [SMILES: [2H]c1c([2H])c([2H])c(-n2c3ccccc3c3cc(B(O)O)ccc32)c([2H])c1[2H]] | 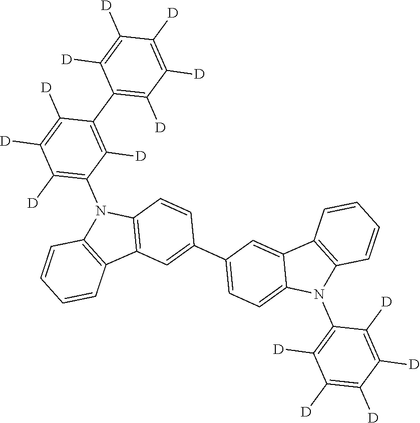 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]] 80% |
| 2-54 | 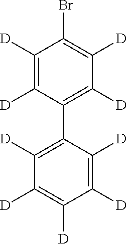 [SMILES: [2H]c1c([2H])c([2H])c(-c2c([2H])c([2H])c(Br)c([2H])c2[2H])c([2H])c1[2H]] | 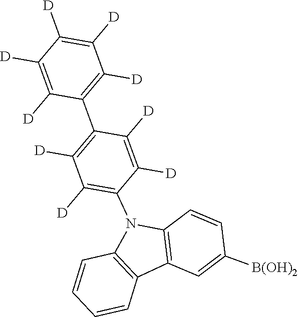 [SMILES: [2H]c1c([2H])c([2H])c(-c2c([2H])c([2H])c(-n3c4ccccc4c4cc(B(O)O)ccc43)c([2H])c2[2H])c([2H])c1[2H]] | 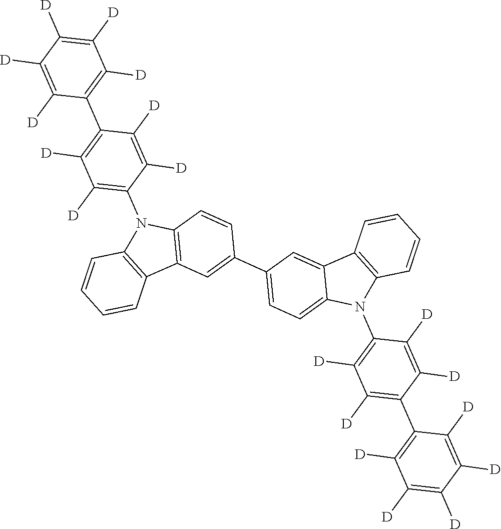 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]] 79% |
| 2-55 | 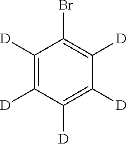 [SMILES: [2H]c1c([2H])c([2H])c(Br)c([2H])c1[2H]] | 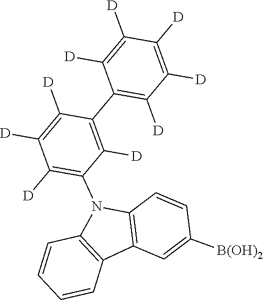 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 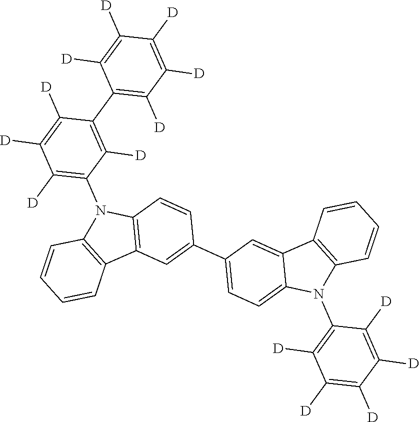 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]] 78% |
| 2-57 | 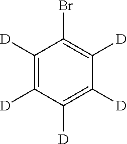 [SMILES: [2H]c1c([2H])c([2H])c(Br)c([2H])c1[2H]] | 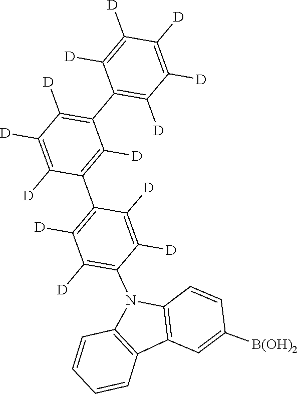 [SMILES: [2H]c1c([2H])c([2H])c(-c2c([2H])c([2H])c([2H])c(-c3c([2H])c([2H])c(-n4c5ccccc5c5cc(B(O)O)ccc54)c([2H])c3[2H])c2[2H])c([2H])c1[2H]] | 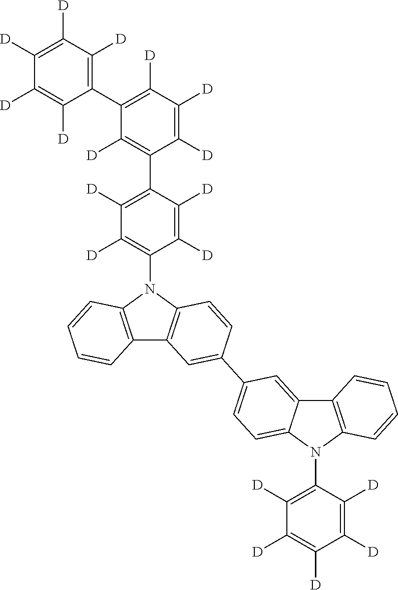 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c2[2H])c([2H])c1[2H]] 79% |
| 2-58 | 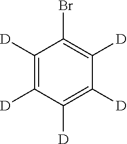 [SMILES: [2H]c1c([2H])c([2H])c(Br)c([2H])c1[2H]] | 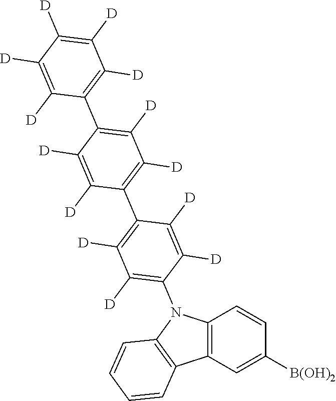 | 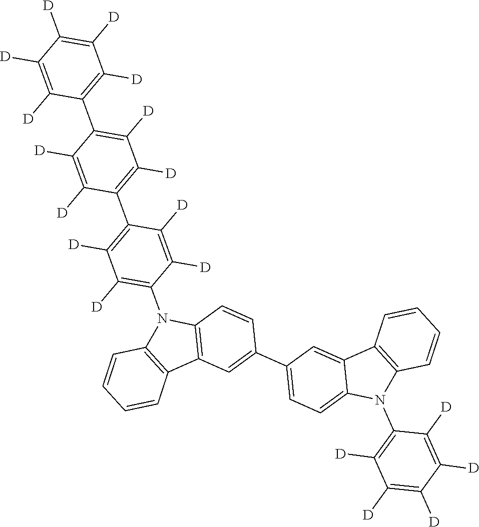 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c([2H])c2[2H])c([2H])c1[2H]] 80% |
| 2-60 | 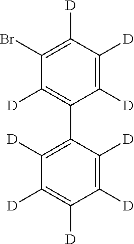 [SMILES: [2H]c1c([2H])c([2H])c(-c2c([2H])c([2H])c([2H])c(Br)c2[2H])c([2H])c1[2H]] | 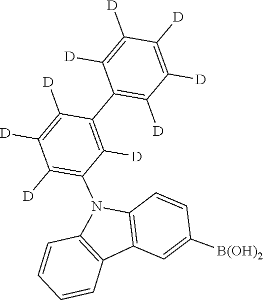 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 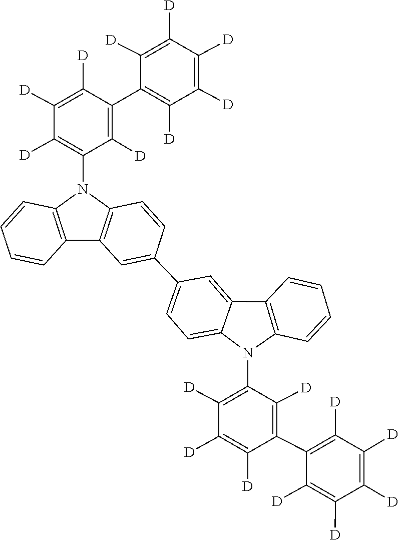 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]] 81% |
| 2-61 | 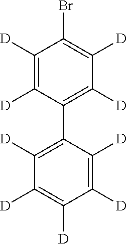 [SMILES: [2H]c1c([2H])c([2H])c(-c2c([2H])c([2H])c(Br)c([2H])c2[2H])c([2H])c1[2H]] | 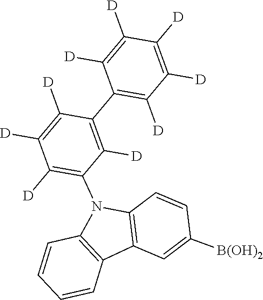 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 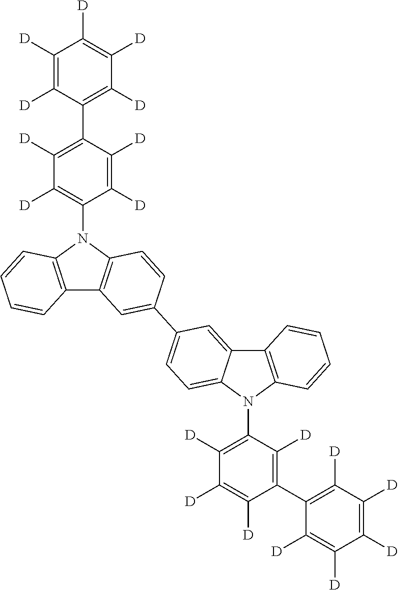 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]] 83% |
| 2-62 | 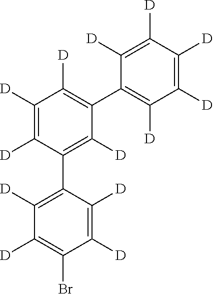 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 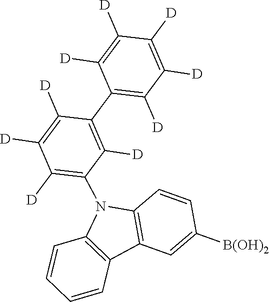 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 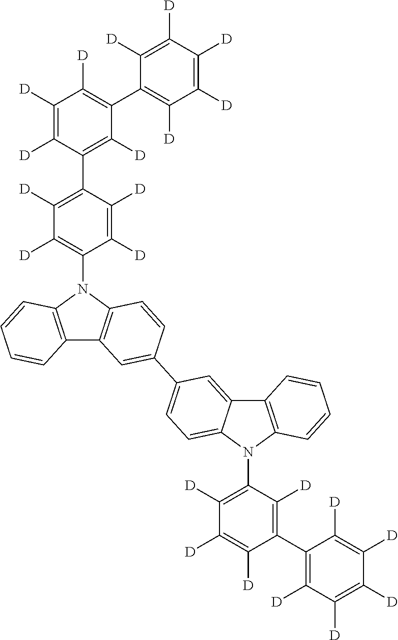 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c2[2H])c([2H])c1[2H]] 82% |
| 2-63 | 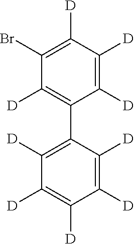 [SMILES: [2H]c1c([2H])c([2H])c(-c2c([2H])c([2H])c([2H])c(Br)c2[2H])c([2H])c1[2H]] | 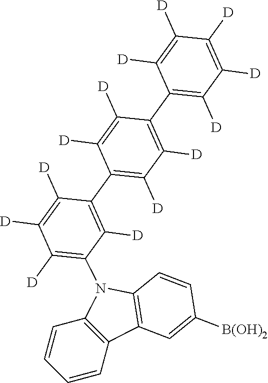 [SMILES: [2H]c1c([2H])c([2H])c(-c2c([2H])c([2H])c(-c3c([2H])c([2H])c([2H])c(-n4c5ccccc5c5cc(B(O)O)ccc54)c3[2H])c([2H])c2[2H])c([2H])c1[2H]] | 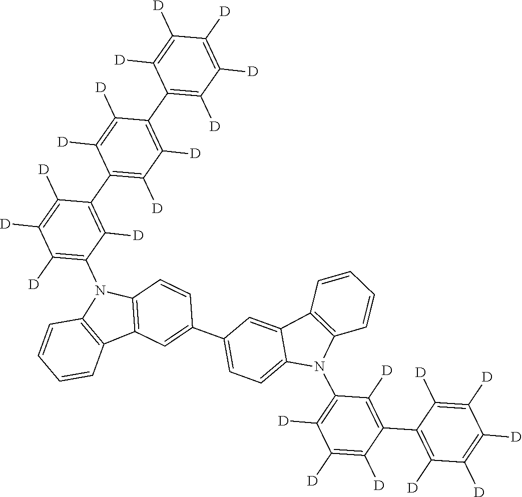 [SMILES: [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c3[2H])c([2H])c2[2H])c([2H])c1[2H]] 84% |
| 2-64 | 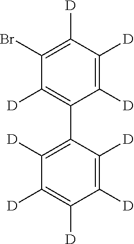 [SMILES: [2H]c1c([2H])c([2H])c(-c2c([2H])c([2H])c([2H])c(Br)c2[2H])c([2H])c1[2H]] | 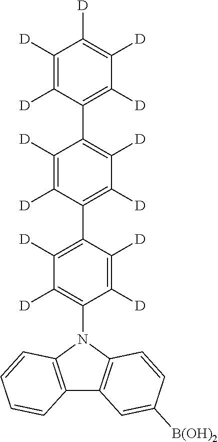 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 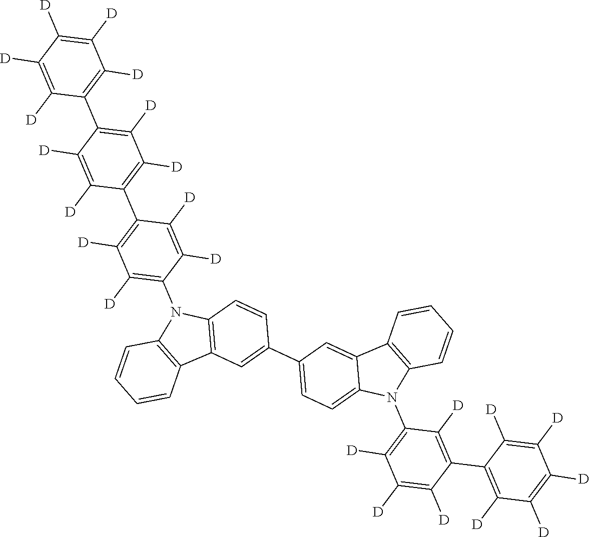 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H]] 80% |
| 2-67 | 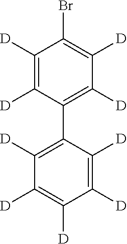 [SMILES: [2H]c1c([2H])c([2H])c(-c2c([2H])c([2H])c(Br)c([2H])c2[2H])c([2H])c1[2H]] | 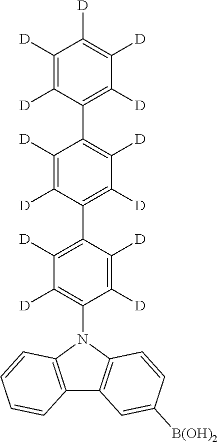 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 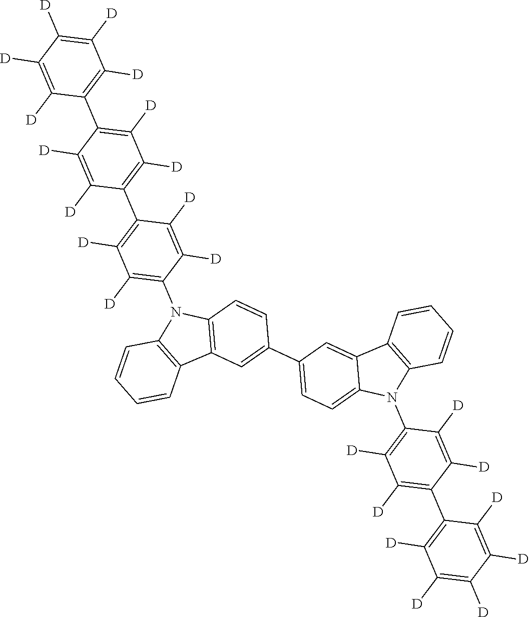 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H]] 81% |
| 2-69 | 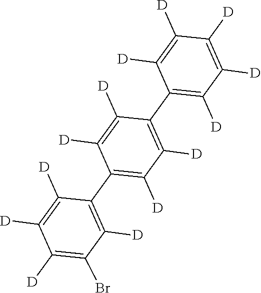 [SMILES: [2H]c1c([2H])c([2H])c(-c2c([2H])c([2H])c(-c3c([2H])c([2H])c([2H])c(Br)c3[2H])c([2H])c2[2H])c([2H])c1[2H]] | 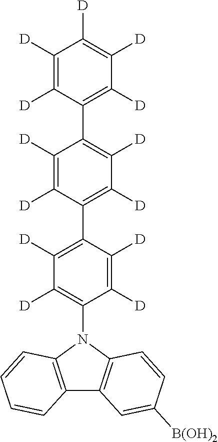 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 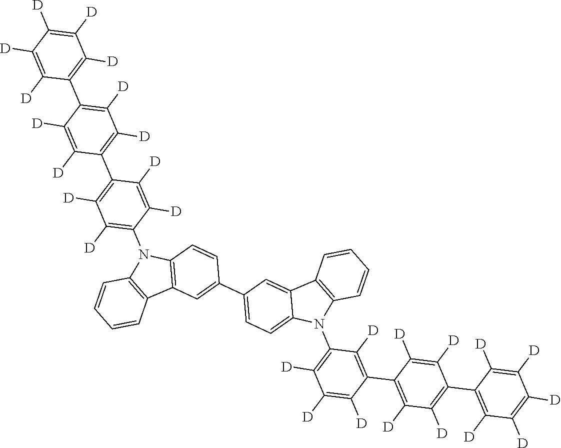 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c([2H])c3[2H])c([2H])c2[2H])c([2H])c1[2H]] 82% |
| 2-72 | 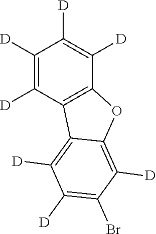 [SMILES: [2H]c1c([2H])c([2H])c2c(oc3c([2H])c(Br)c([2H])c([2H])c32)c1[2H]] | 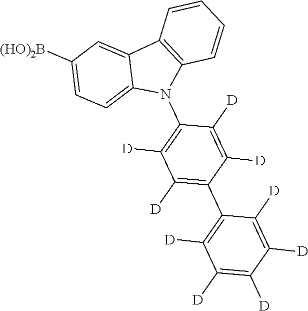 [SMILES: [2H]c1c([2H])c([2H])c(-c2c([2H])c([2H])c(-n3c4ccccc4c4cc(B(O)O)ccc43)c([2H])c2[2H])c([2H])c1[2H]] | 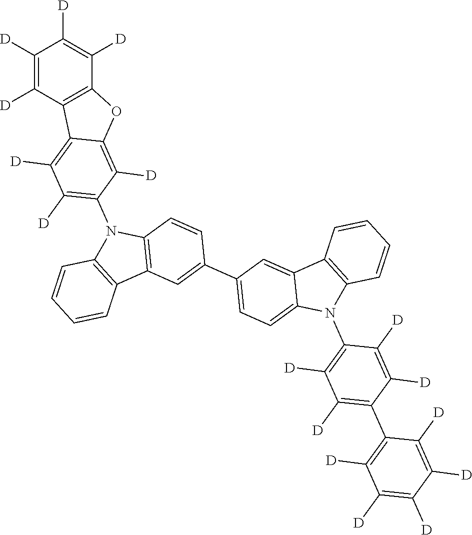 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]] 82% |

### [Preparation Example 3] Preparation of Compound 2-73 and the Like

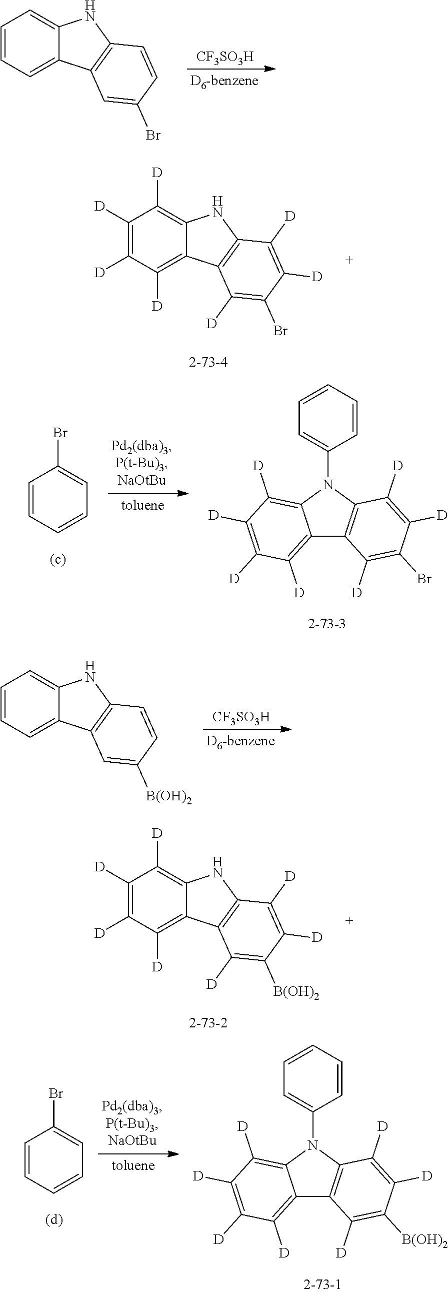

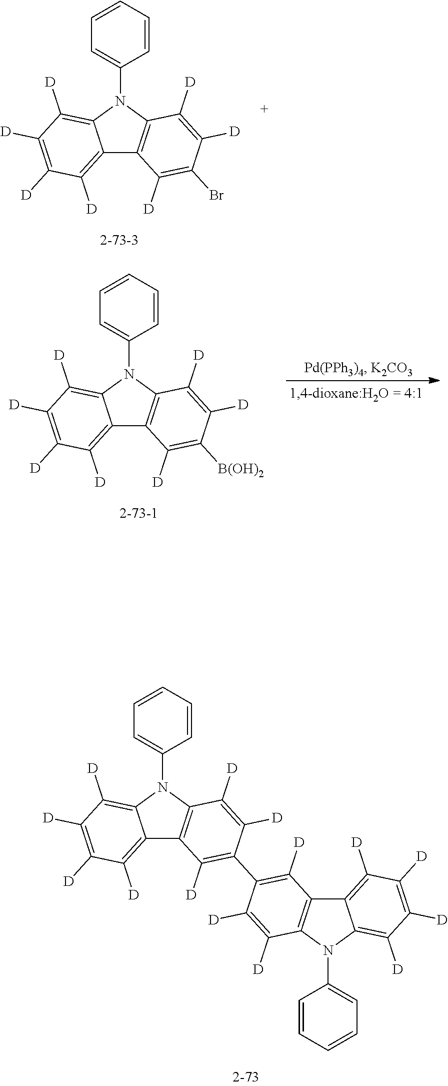 [SMILES: [2H]c1c([2H])c([2H])c2c(c1[2H])c1c([2H])c(Br)c([2H])c([2H])c1n2-c1ccccc1 ; [2H]c1c([2H])c([2H])c2c(c1[2H])c1c([2H])c(B(O)O)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

### 1) Preparation of Compound 2-73-4

10 g (40.23 mmol) of 3-bromo-9H-carbazole, 1,000 mL of D₆-benzene, and 170 g (1,075 mmol) of triflic acid (CF₃SO₃H) were put into a reaction flask, and the resulting mixture was stirred at 50° C.

After the reaction was completed, the resulting product was neutralized with D₂O and then extracted with an aqueous sodium carbonate (Na₂CO₃) solution and dichloromethane (DCM) at room temperature, the organic layer was dried over anhydrous magnesium sulfate (MgSO₄), and then the solvent was removed using a rotary evaporator. The reaction product was purified by column chromatography (dichloromethane:hexane=1:2) and recrystallized with methanol to obtain 10 g (yield 98%) of Target Compound 2-73-4.

### 2) Preparation of Compound 2-73-3

After 10 g (39.5 mmol) of Compound 2-73-4, 12.4 g (79 mmol) of 2-bromobenzene (c), 1.81 g (1.98 mmol) of tris(dibenzylideneacetone)dipalladium (Pd₂(dba)₃), 1.93 mL (7.9 mmol) of tri-tert-butylphosphine (P(t-Bu)₃), and 11.4 g (118.51 mmol) of sodium tert-butoxide (NatOBu) were put into a reaction flask, 100 mL of toluene was added thereto, and the resulting mixture was heated at 135° C. for 15 hours. When the reaction was terminated, the resulting product was extracted with methylene chloride (M C) and water, and then purified by column chromatography to obtain 11 g (yield 84%) of Compound 2-73-3.

### 3) Preparation of Compound 2-73-2

11 g (47.3 mmol) of 9H-carbazol-3-ylboronic acid, 1,000 mL of D₆-benzene, and 170 g (1,075 mmol) of triflic acid (CF₃SO₃H) were put into a reaction flask, and the resulting mixture was stirred at 50° C.

After the reaction was completed, the resulting product was neutralized with D₂O and then extracted with an aqueous sodium carbonate (Na₂CO₃) solution and dichloromethane (DCM) at room temperature, the organic layer was dried over anhydrous magnesium sulfate (MgSO₄), and then the solvent was removed using a rotary evaporator. The reaction product was purified by column chromatography (dichloromethane:hexane=1:2) and recrystallized with methanol to obtain 9 g (yield 87%) of Target Compound 2-73-2.

### 4) Preparation of Compound 2-73-1

After 9 g (41.3 mmol) of Compound 2-73-2, 12.9 g (82.5 mmol) of bromobenzene (d), 1.89 g (2.06 mmol) of tris(dibenzylideneacetone)dipalladium (Pd₂(dba)₃), 2 mL (8.25 mmol) of tri-tert-butylphosphine (P(t-Bu)₃), and 7.93 g (82.574 mmol) of sodium tert-butoxide (NatOBu) were put into a reaction flask, 100 mL of toluene was added thereto, and the resulting mixture was heated at 135° C. for 10 hours. When the reaction was terminated, the resulting product was extracted with methylene chloride (M C) and water, and then purified by column chromatography to obtain 10 g (yield 82%) of Compound 2-73-1.

### 5) Preparation of Compound 2-73

10 g (30.37 mmol) of Compound 2-73-3, 10 g (60.75 mmol) of Compound 2-73-1, 1.39 g (1.52 mmol) of tetrakis(triphenylphosphine)palladium(0) (Pd(PPh₃)₄), and 12.59 g (91.13 mmol) of potassium carbonate (K₂CO₃) were put into a reaction flask, and then added to 140 mL of 1,4-dioxane and 35 mL of distilled water, and the resulting mixture was stirred at 120° C. for 4 hours.

Thereafter, a solid produced by lowering the temperature to room temperature was washed with distilled water and methanol to obtain 13 g (yield 85%) of Compound 2-73.

The target compounds in the following Table 5 were synthesized in the same manner as in the preparation of Preparation Example 3, except that Compound c in the following Table 5 was used instead of bromobenzene (c), and Compound d in the following Table 5 was used instead of bromobenzene (d) in Preparation Example 3.

TABLE 5

| Compound | | | Target Compound |
|---|---|---|---|
| No. | Compound c | Compound d | (Yield %) |
| 2-74 | 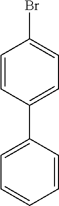 [SMILES: Brc1ccc(-c2ccccc2)cc1] | 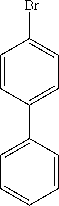 [SMILES: Brc1ccc(-c2ccccc2)cc1] | 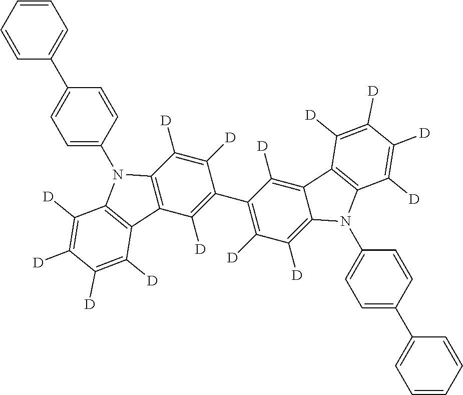 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1] 82% |
| 2-75 | 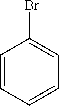 | 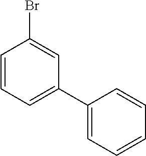 [SMILES: Brc1cccc(-c2ccccc2)c1] | 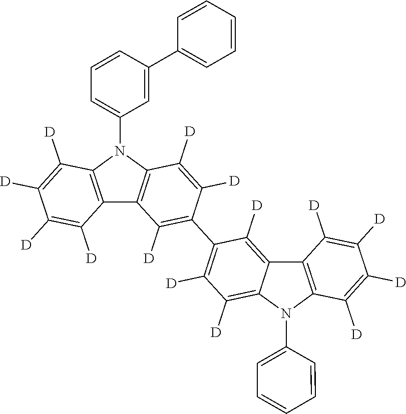 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccccc1] 84% |
| 2-76 | 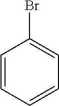 | 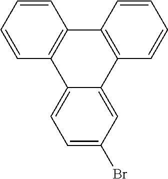 [SMILES: Brc1ccc2c3ccccc3c3ccccc3c2c1] | 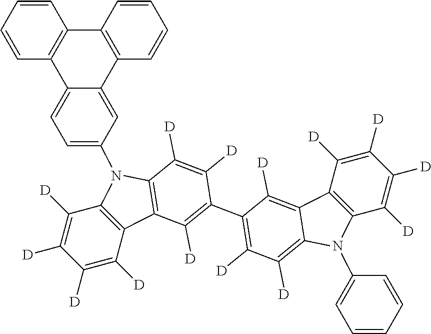 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1ccccc1] 80% |
| 2-77 | 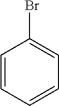 | 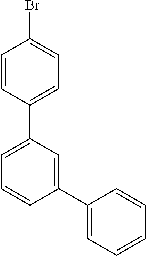 [SMILES: Brc1ccc(-c2cccc(-c3ccccc3)c2)cc1] | 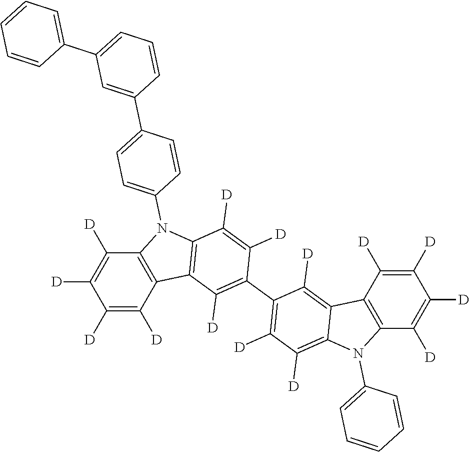 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccccc1] 81% |
| 2-78 | 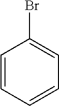 | 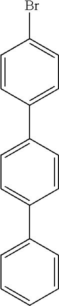 [SMILES: Brc1ccc(-c2ccc(-c3ccccc3)cc2)cc1] | 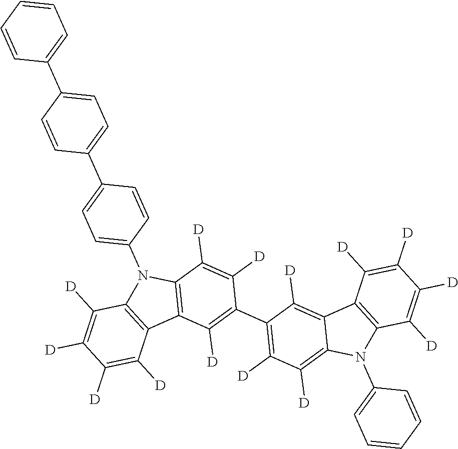 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccccc1] 82% |
| 2-80 | 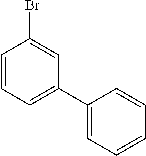 [SMILES: Brc1cccc(-c2ccccc2)c1] | 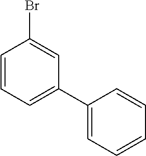 [SMILES: Brc1cccc(-c2ccccc2)c1] | 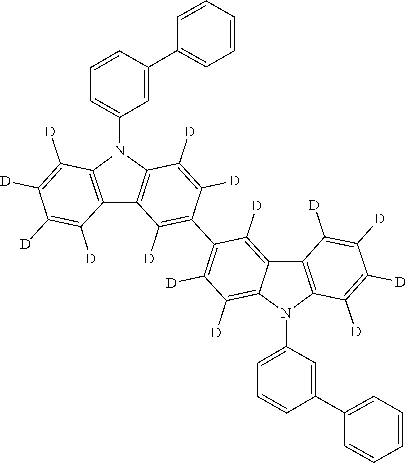 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1cccc(-c2ccccc2)c1] 84% |
| 2-81 | 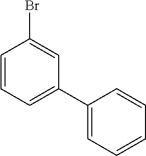 [SMILES: Brc1cccc(-c2ccccc2)c1] | 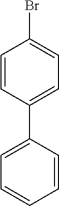 [SMILES: Brc1ccc(-c2ccccc2)cc1] | 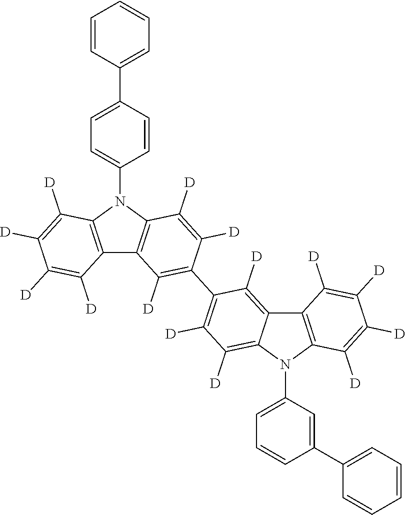 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccccc2)cc1] 80% |
| 2-82 | 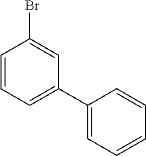 [SMILES: Brc1cccc(-c2ccccc2)c1] | 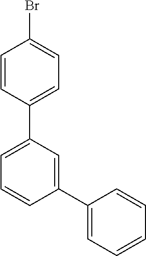 [SMILES: Brc1ccc(-c2cccc(-c3ccccc3)c2)cc1] | 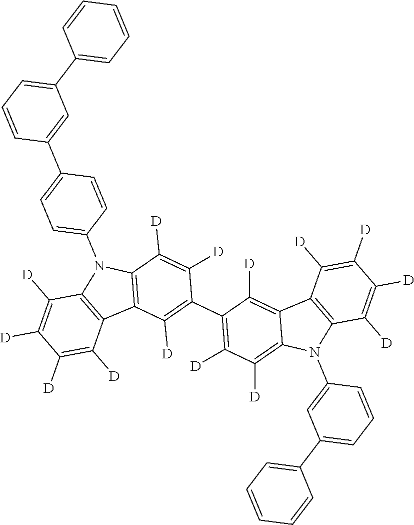 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2cccc(-c3ccccc3)c2)cc1] 81% |
| 2-86 | 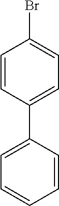 [SMILES: Brc1ccc(-c2ccccc2)cc1] | 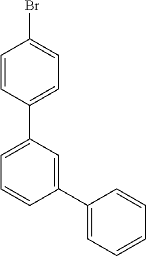 [SMILES: Brc1ccc(-c2cccc(-c3ccccc3)c2)cc1] | 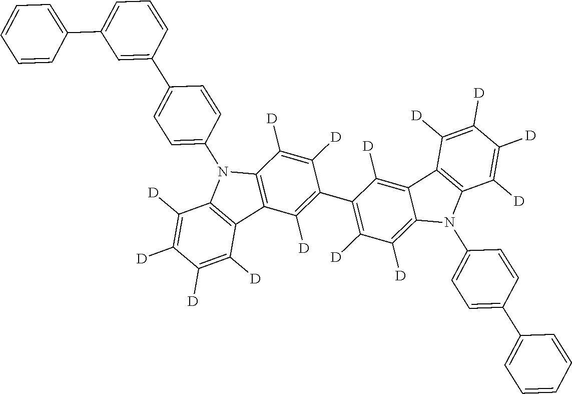 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccc(-c2ccccc2)cc1] 81% |
| 2-87 | 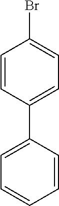 [SMILES: Brc1ccc(-c2ccccc2)cc1] | 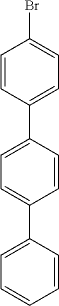 [SMILES: Brc1ccc(-c2ccc(-c3ccccc3)cc2)cc1] | 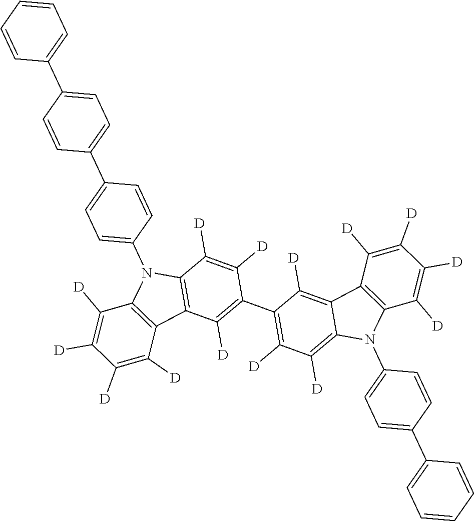 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccccc2)cc1] 80% |
| 2-114 | 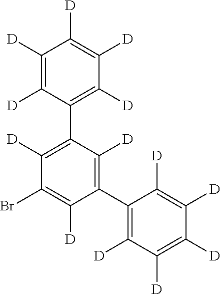 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 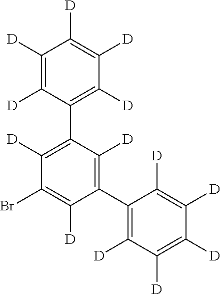 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 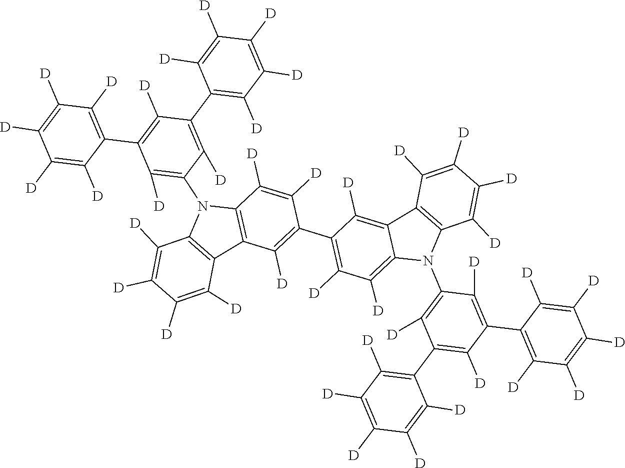 [SMILES: [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c(-c6c([2H])c([2H])c([2H])c([2H])c6[2H])c([2H])c(-c6c([2H])c([2H])c([2H])c([2H])c6[2H])c5[2H])c([2H])c([2H])c43)c2[2H])c([2H])c1[2H]] 81% |

### [Preparation Example 4] Preparation of Compound 2-93

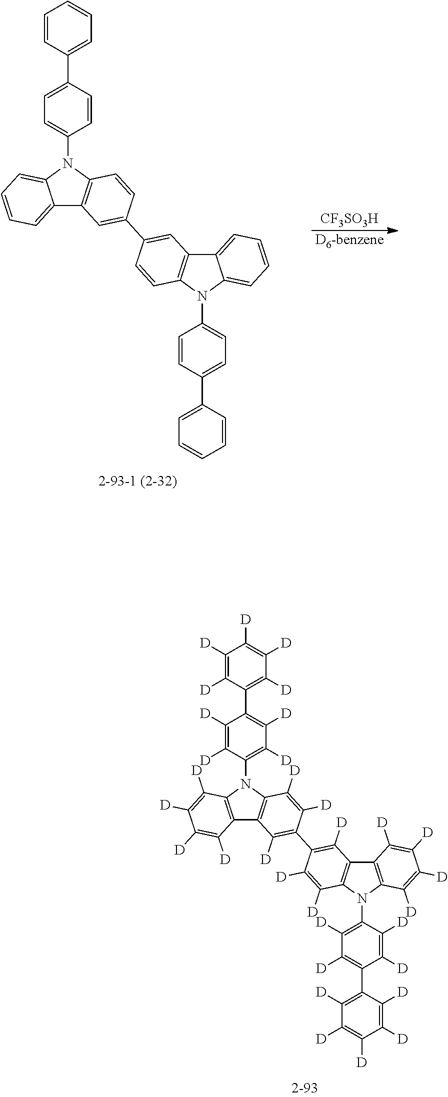 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; "CF3SO3H" ; "D6-benzene" ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

10 g (15.7 mmol) of Compound 2-93-1 (Compound 2-32), 1,000 mL of D₆-benzene, and 170 g (1,075 mmol) of triflic acid (CF₃SO₃H) were put into a reaction flask, and the resulting mixture was stirred at 50° C.

After the reaction was completed, the resulting product was neutralized with D₂O and then extracted with an aqueous sodium carbonate (Na₂CO₃) solution and dichloromethane (DCM) at room temperature, the organic layer was dried over anhydrous magnesium sulfate (MgSO₄), and then the solvent was removed using a rotary evaporator. The reaction product was purified by column chromatography (dichloromethane:hexane=1:2) and recrystallized with methanol to obtain 10.0 g (yield 95%) of Target Compound 2-93.

The target compounds in the following Table 6 were synthesized in the same manner as in the preparation of Preparation Example 3, except that Compound c in the following Table 6 was used instead of bromobenzene (c), and Compound d in the following Table 6 was used instead of bromobenzene (d) in Preparation Example 3.

TABLE 6

| Compound | | | Target Compound |
|---|---|---|---|
| No. | Compound c | Compound d | (Yield %) |
| 2-93 | 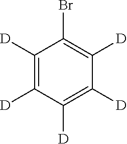 [SMILES: [2H]c1c([2H])c([2H])c(Br)c([2H])c1[2H]] | 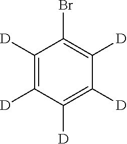 [SMILES: [2H]c1c([2H])c([2H])c(Br)c([2H])c1[2H]] | 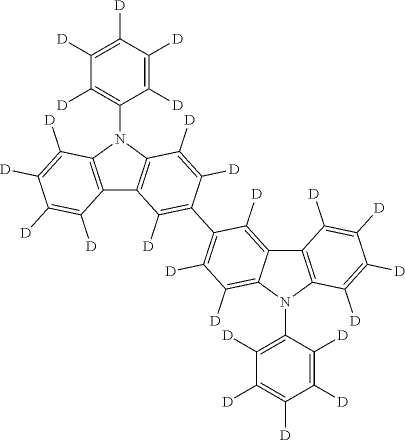 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]] 83% |
| 2-94 | 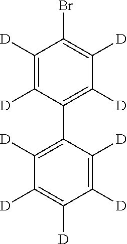 [SMILES: [2H]c1c([2H])c([2H])c(-c2c([2H])c([2H])c(Br)c([2H])c2[2H])c([2H])c1[2H]] | 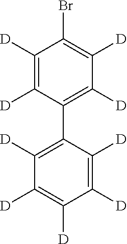 [SMILES: [2H]c1c([2H])c([2H])c(-c2c([2H])c([2H])c(Br)c([2H])c2[2H])c([2H])c1[2H]] | 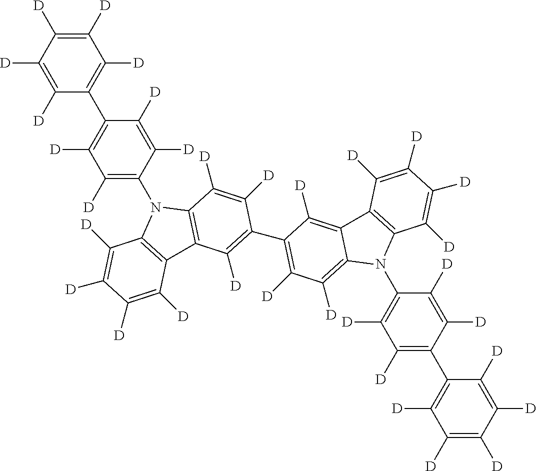 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]] 82% |
| 2-95 | 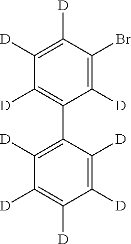 [SMILES: [2H]c1c([2H])c([2H])c(-c2c([2H])c([2H])c([2H])c(Br)c2[2H])c([2H])c1[2H]] | 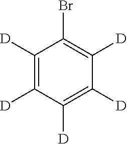 [SMILES: [2H]c1c([2H])c([2H])c(Br)c([2H])c1[2H]] | 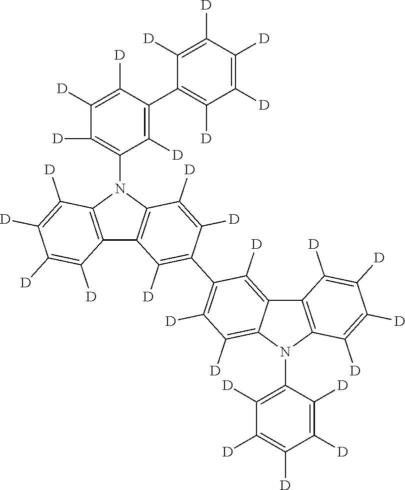 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] 84% |
| 2-97 | 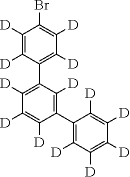 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 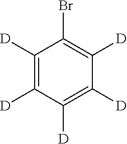 [SMILES: [2H]c1c([2H])c([2H])c(Br)c([2H])c1[2H]] | 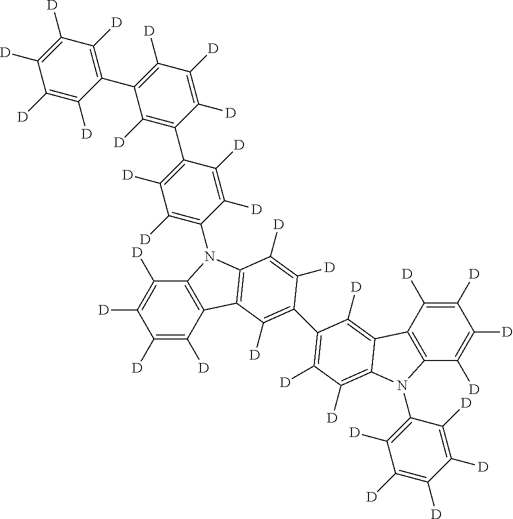 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c2[2H])c([2H])c1[2H]] 80% |
| 2-98 | 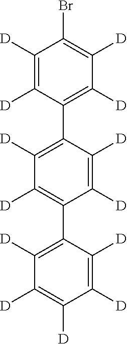 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 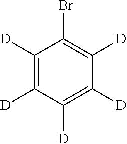 [SMILES: [2H]c1c([2H])c([2H])c(Br)c([2H])c1[2H]] | 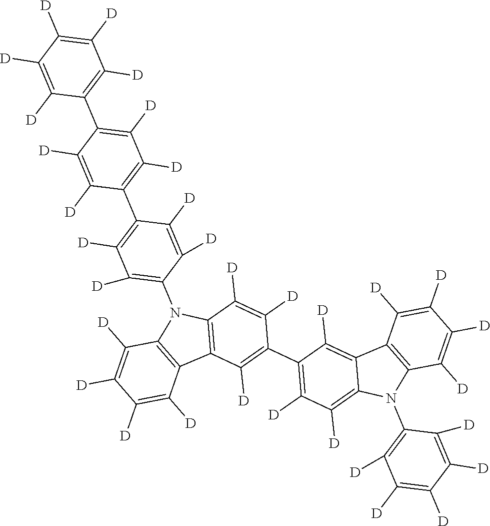 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c([2H])c2[2H])c([2H])c1[2H]] 81% |
| 2-99 | 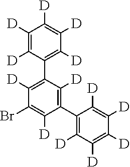 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 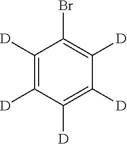 [SMILES: [2H]c1c([2H])c([2H])c(Br)c([2H])c1[2H]] | 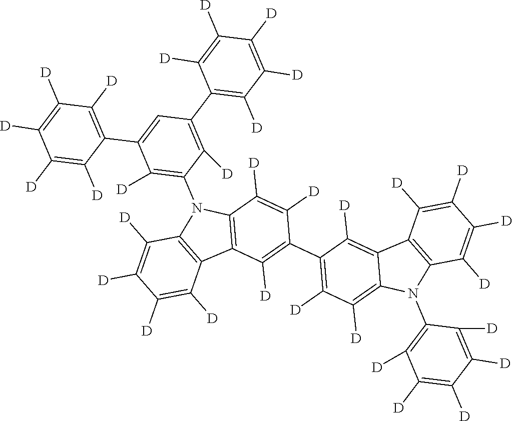 [SMILES: [2H]c1c([2H])c([2H])c(-c2cc(-c3c([2H])c([2H])c([2H])c([2H])c3[2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] 81% |
| 2-100 | 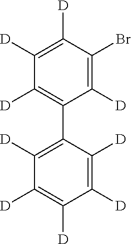 [SMILES: [2H]c1c([2H])c([2H])c(-c2c([2H])c([2H])c([2H])c(Br)c2[2H])c([2H])c1[2H]] | 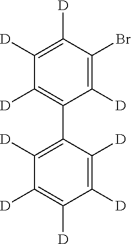 [SMILES: [2H]c1c([2H])c([2H])c(-c2c([2H])c([2H])c([2H])c(Br)c2[2H])c([2H])c1[2H]] | 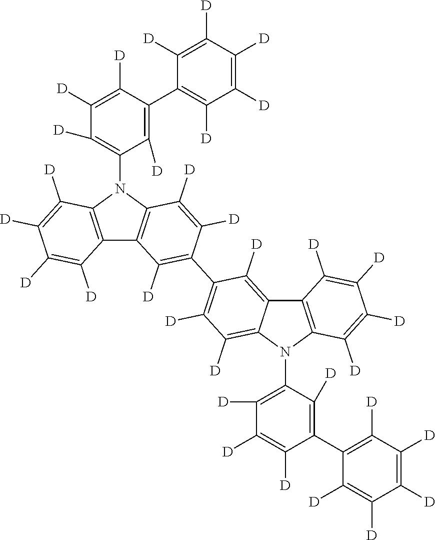 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c2[2H])c([2H])c1[2H]] 82% |
| 2-101 | 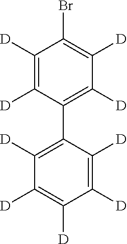 [SMILES: [2H]c1c([2H])c([2H])c(-c2c([2H])c([2H])c(Br)c([2H])c2[2H])c([2H])c1[2H]] | 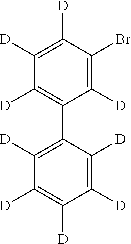 [SMILES: [2H]c1c([2H])c([2H])c(-c2c([2H])c([2H])c([2H])c(Br)c2[2H])c([2H])c1[2H]] | 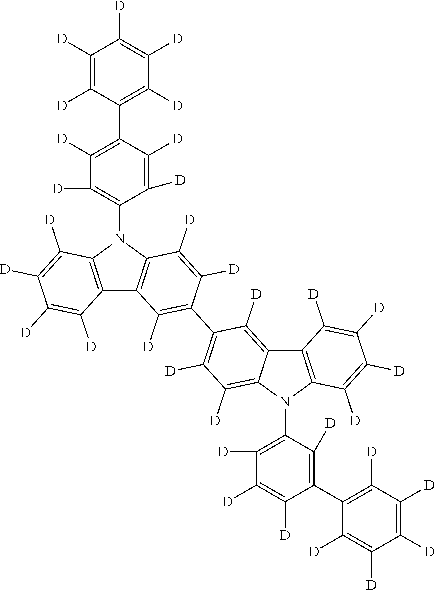 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]] 82% |
| 2-102 | 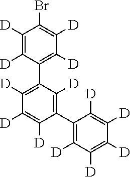 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 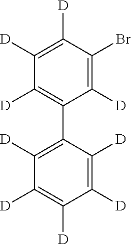 [SMILES: [2H]c1c([2H])c([2H])c(-c2c([2H])c([2H])c([2H])c(Br)c2[2H])c([2H])c1[2H]] | 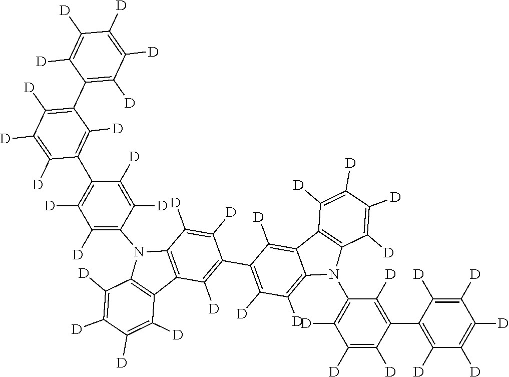 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c2[2H])c([2H])c1[2H]] 84% |
| 2-104 | 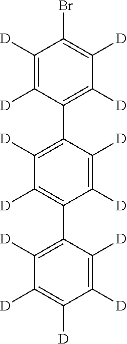 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 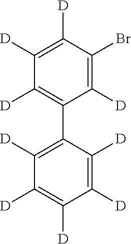 [SMILES: [2H]c1c([2H])c([2H])c(-c2c([2H])c([2H])c([2H])c(Br)c2[2H])c([2H])c1[2H]] | 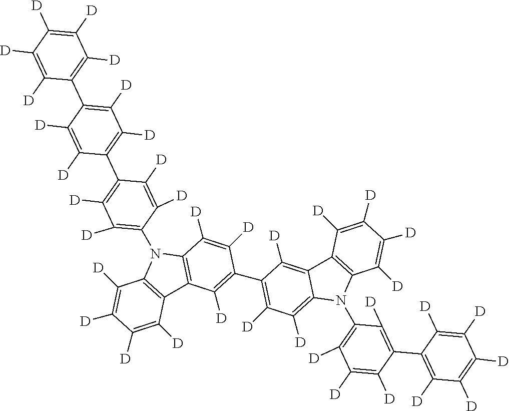 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c([2H])c2[2H])c([2H])c1[2H]] 80% |
| 2-112 | 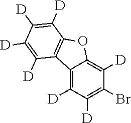 [SMILES: [2H]c1c([2H])c([2H])c2c(oc3c([2H])c(Br)c([2H])c([2H])c32)c1[2H]] | 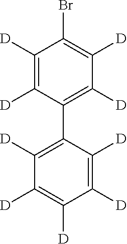 [SMILES: [2H]c1c([2H])c([2H])c(-c2c([2H])c([2H])c(Br)c([2H])c2[2H])c([2H])c1[2H]] | 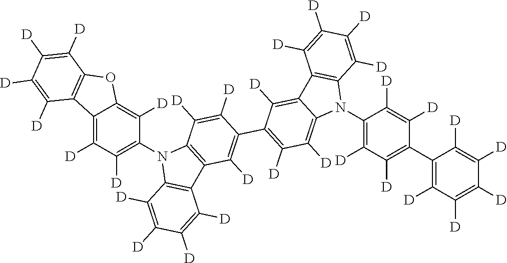 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(oc7c([2H])c([2H])c([2H])c([2H])c76)c5[2H])c([2H])c([2H])c43)c([2H])c2[2H])c([2H])c1[2H]] 84% |

Compounds were prepared in the same manner as in the Preparation Examples, and the synthesis confirmation results thereof are shown in the following Tables 7 and 8. Table 7 shows the measured values of ¹H NMR (CDCl₃, 400 MHz), and Table 8 shows the measured values of field desorption mass spectrometry (FD-MS).

TABLE 7

| Compound | |
|---|---|
| No. | ¹H NMR(CDCl₃, 400 MHz) |
| 2-1 | δ = 8.55(d, 1H), 8.30(d, 1H), 8.19-8.13(m, 2H), 7.99-7.89(m, 4H), 7.77(d, |
| | 1H), 7.62-7.50(m, 12H), 7.35(t, 1H), 7.20-7.16(m, 2H) |
| 2-2 | δ = 8.55(d, 1H), 8.30(d, 1H), 8.19-8.13(m, 2H), 7.99-7.89(m, 6H), 7.80- |
| | 7.77(m, 2H), 7.62-7.35(m, 10H), 7.20-7.16(m, 6H) |
| 2-3 | δ = 8.55(d, 1H), 8.18-8.09(m, 3H), 8.00-7.87(m, 3H), 7.77(s, 2H), 7.58- |
| | 7.25(m, 18H) |
| 2-4 | δ = 8.55(d, 1H), 8.18-8.12(m, 2H), 8.00-7.84(m, 3H), 7.79-7.77(m, 4H), 7.68- |
| | 7.25(m, 22H) |
| 2-5 | δ = 8.55(d, 1H), 8.30(d, 1H), 8.21-8.13(m, 3H), 7.99-7.89(m, 4H), 7.77- |
| | 7.35(m, 17H), 7.25-7.16(m, 6H) |
| 2-6 | δ = 8.55(d, 1H), 8.30(d, 1H), 8.19-8.13(m, 2H), 7.94-7.89(m, 8H), 7.77- |
| | 7.75(m, 3H), 7.62-7.35(m, 11H), 7.25-7.16(m, 6H) |
| 2-7 | δ = 8.55(d, 1H), 8.18-8.09(m, 4H), 8.00-7.94(m, 2H), 7.87(m, 1H), 7.77(m, |
| | 2H), 7.69-7.63(m, 2H), 7.52-7.25(m, 20H) |
| 2-11 | δ = 8.55(d, 1H), 8.30(d, 1H), 8.19-8.13(m, 2H), 7.99-7.89(m, 5H), 7.77(d, |
| | 1H), 7.58-7.28(m, 16H), 1.69(s, 6H) |
| 2-16 | δ = 9.05(s, 1H), 8.55(d, 1H), 8.33-8.13(m, 7H), 7.99-7.89(m, 5H), 7.77- |
| | 7.50(m, 13H), 7.35(t, 1H), 7.20-7.16(m, 2H) |
| 2-19 | δ = 8.55(d, 1H), 8.30(d, 1H), 8.19-8.13(m, 2H), 7.99-7.89(m, 8H), 7.80- |
| | 7.77(m, 3H), 7.58(d, 1H), 7.50-7.35(m, 6H), 7.20-7.16(m, 10H) |
| 2-20 | § = 8.55(d, 1H), 8.30(d, 1H), 8.21-8.13(m, 3H), 7.99-7.89(m, 6H), 7.80- |
| | 7.35(m, 15H), 7.20-7.16(6H) |
| 2-21 | δ = 8.55(d, 1H), 8.30(d, 1H), 8.19-8.13(m, 2h), 7.99-7.89(m, 10H), 7.80- |
| | 7.75(m, 4H), 7.50-7.35(m, 8H), 7.20-7.16(m, 6H) |
| 2-22 | δ = 8.55(d, 1H), 8.30(d, 1H), 8.21-8.13(m, 3H), 7.99-7.89(m, 6H), 7.80- |
| | 7.35(m, 15H), 7.25-7.16(10H) |
| 2-23 | δ = 8.55(d, 1H), 8.30(d, 1H), 8.19-8.13(m, 2h), 7.99-7.89(m, 10H), 7.80- |
| | 7.75(m, 4H), 7.50-7.35(m, 8H), 7.25-7.16(m, 10H) |
| 2-26 | δ = 8.55(m, 1H), 8.30(d, 1H), 8.21-8.13(m, 4h), 7.99-7.89(m, 4H), 7.77- |
| | 7.35(m, 20H), 7.25-7.16(6H) |
| 2-27 | δ = 8.55(m, 1H), 8.30(d, 1H), 8.21-8.13(m, 4h), 7.99-7.89(m, 4H), 7.77- |
| | 7.35(m, 20H), 7.20-7.16(2H) |
| 2-28 | δ = 8.55(m, 1H), 8.18-8.09(m, 3H), 8.00-8.79(m, 2H), 7.87(m, 1H), 7.79- |
| | 7.77(m, 4H), 7.69-7.63(m, 4H), 7.52-7.25(m, 12H) |
| 2-29 | δ = 8.55(m, 1H), 8.18-8.09(m, 3H), 8.00-7.94(m, 2H0, 7.87(m, 1H), 7.87(m, |
| | 1H), 7.79-7.77(m, 4H), 7.69-7.63(m, 4H), 7.52-7.25(m, 21H) |
| 2-30 | δ = 8.55(m, 1H), 8.31-8.30(m, 3H), 8.21-8.13(m, 3h), 7.99-7.89(m, 3H), |
| | 7.75-7.35(m, 22H), 7.20-7.16(m, 2H) |
| 2-32 | δ = 8.55(m, 1H), 8.18-8.12(m, 2H), 8.00-7.87(m, 3H), 7.79-7.77(m, 6H), |
| | 7.69-7.63(m, 6H), 7.52-7.25(m, 14H) |
| 2-33 | δ = 8.55(m, 1H), 8.30(d, 1H), 8.21-8.13(m, 3H), 7.99-7.89(m, 8H), 7.77- |
| | 7.35(m, 17H), 7.25-7.16(6H) |
| 2-34 | § = 8.55(m, 1H), 8.18-8.12(m, 2H), 8.00-7.87(m, 3H), 7.79-7.77(m, 6H), |
| | 7.67-7.63(m, 6H), 7.52-7.25(m, 18H) |
| 2-38 | δ = 8.55(m, 1H), 8.18-8.12(m, 2H), 8.05-7.87(m, 6H), 7.79-7.77(m, 4H), |
| | 7.69-7.63(m, 4H), 7.52-7.25(m, 23H) |
| 2-40 | δ = 8.55(m, 1H), 8.30(d, 1H), 8.19-8.13(m, 2H), 8.03-7.75(m, 15H), 7.58- |
| | 7.35(m, 9H), 7.25-7.16(m, 6H) |
| 2-53 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-54 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-55 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-57 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-58 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-60 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-61 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-62 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-63 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-64 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-67 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-69 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-72 | δ = 8.55(m, 1H), 8.30(d, 1H), 8.19-8.13(m, 2H), 7.99-7.89(m, 4H), 7.77(d, |
| | 1H), 7.58-7.50(m, 2H), 7.35(t, 1H), 7.20-7.16(m, 2H) |
| 2-73 | δ = 7.62-7.50(m, 10H) |
| 2-74 | δ = 7.79(m, 4H), 7.68(m. 4H), 7.52-7.41(m, 10H) |
| 2-75 | δ = 8.21(s, 1H) 7.75-7.41(m, 13H) |
| 2-76 | δ = 9.05(s, 1H), 8.33-8.25(m, 4H), 7.94(d, 1H), 7.70-7.50(m, 10H) |
| 2-77 | δ = 7.79(m, 2H), 7.70-7.68(m, 3H), 7.58-7.41(m, 13H) |
| 2-78 | δ = 7.92-7.91(m, 4H), 7.75(d, 2H), 7.62-7.41(m, 8H), 7.25(s, 4H) |
| 2-80 | δ = 8.21(s, 2H), 7.75-7.60(m, 8H), 7.49-7.41(8H) |
| 2-81 | δ = 8.21(s, 1H), 7.92-7.91(m, 4H), 7.75-7.60(m, 6H), 7.49-7.41(m, 7H) |
| 2-82 | δ = 8.21(s, 1H), 7.94-7.91(m, 5H), 7.75-7.61(m, 9H), 7.49-7.41(m, 7H) |
| 2-86 | δ = 7.94-7.91(m, 9H), 7.75-7.73(m, 5H), 7.61(d, 2H), 7.49-7.41(m, 6H) |
| 2-87 | δ = 7.92-7.91(m, 8H), 7.75(d, 4H), 7.49-7.41(m, 6H), 7.25(s, 4H) |

TABLE 8

| Compound | | Compound | |
|---|---|---|---|
| No. | FD-MS | No. | FD-MS |
| 2-1 | m/z = 484.59(C₃₆H₂₄N₂ = 484.19) | 2-2 | m/z = 560.69(C₄₂H₂₈N₂ = 560.23) |
| 2-3 | m/z = 560.69(C₄₂H₂₈N₂ = 560.23) | 2-4 | m/z = 560.69(C₄₂H₂₈N₂ = 560.23) |
| 2-5 | m/z = 636.78(C₄₈H₃₂N₂ = 636.26) | 2-6 | m/z = 636.78(C₄₈H₃₂N₂ = 636.26) |
| 2-7 | m/z = 636.78(C₄₈H₃₂N₂ = 636.26) | 2-8 | m/z = 543.65(C₄₀H₂₆N₂ = 543.21) |
| 2-9 | m/z = 543.65(C₄₀H₂₆N₂ = 543.21) | 2-10 | m/z = 600.75(C₄₅H₃₅N₂ = 600.26) |
| 2-11 | m/z = 600.75(C₄₅H₃₅N₂ = 600.26) | 2-12 | m/z = 724.89(C₅₅H₃₆N₂ = 724.29) |
| 2-13 | m/z = 724.89(C₅₅H₃₆N₂ = 724.29) | 2-14 | m/z = 724.89(C₅₅H₃₆N₂ = 724.29) |
| 2-15 | m/z = 724.89(C₅₅H₃₆N₂ = 724.29) | 2-16 | m/z = 634.77(C₄₈H₃₀N₂ = 634.24) |
| 2-17 | m/z = 509.60(C₃₇H₂₃N₃ = 509.19) | 2-18 | m/z = 742.98(C₅₄H₃₈N₂Si = 742.28) |
| 2-19 | m/z = 636.78(C₄₈H₃₂N₂ = 636.26) | 2-20 | m/z = 636.78(C₄₈H₃₂N₂ = 636.26) |
| 2-21 | m/z = 636.78(C₄₈H₃₂N₂ = 636.26) | 2-22 | m/z = 712.88(C₅₄H₃₆N₂ = 712.29) |
| 2-23 | m/z = 712.88(C₅₄H₃₆N₂ = 712.29) | 2-24 | m/z = 712.88(C₅₄H₃₆N₂ = 712.29) |
| 2-25 | m/z = 710.86(C₅₄H₃₄N₂ = 710.27) | 2-26 | m/z = 712.88(C₅₄H₃₆N₂ = 712.29) |
| 2-27 | m/z = 712.88(C₅₄H₃₆N₂ = 712.29) | 2-28 | m/z = 712.88(C₅₄H₃₆N₂ = 712.29) |
| 2-29 | m/z = 712.88(C₅₄H₃₆N₂ = 712.29) | 2-30 | m/z = 712.88(C₅₄H₃₆N₂ = 712.29) |
| 2-31 | m/z = 710.86(C₅₄H₃₄N₂ = 710.27) | 2-32 | m/z = 636.78(C₄₈H₃₂N₂ = 636.26) |
| 2-33 | m/z = 712.88(C₅₄H₃₆N₂ = 712.29) | 2-34 | m/z = 712.88(C₅₄H₃₆N₂ = 712.29) |
| 2-35 | m/z = 788.97(C₆₀H₄₀N₂ = 788.32) | 2-36 | m/z = 686.84(C₅₂H₃₄N₂ = 686.27) |
| 2-37 | m/z = 788.97(C₆₀H₄₀N₂ = 788.32) | 2-38 | m/z = 788.97(C₆₀H₄₀N₂ = 788.32) |
| 2-39 | m/z = 686.84(C₅₂H₃₄N₂ = 686.27) | 2-40 | m/z = 686.84(C₅₂H₃₄N₂ = 686.27) |
| 2-53 | m/z = 494.65(C₃₆H₁₄D₁₀N₂ = 494.26) | 2-54 | m/z = 654.89(C₄₈H₁₄D₁₈N₂ = 654.37) |
| 2-55 | m/z = 574.77(C₄₁H₁₄D₁₄N₂ = 574.31) | 2-56 | m/z = 650.86(C₄₈H₁₄D₁₆N₂ = 650.34) |
| 2-57 | m/z = 654.89(C₄₈H₁₄D₁₈N₂ = 654.37) | 2-58 | m/z = 654.89(C₄₈H₁₄D₁₈N₂ = 654.37) |
| 2-59 | m/z = 654.89(C₄₈H₁₄D₁₈N₂ = 654.37) | 2-60 | m/z = 654.89(C₄₈H₁₄D₁₈N₂ = 654.37) |
| 2-61 | m/z = 654.89(C₄₈H₁₄D₁₈N₂ = 654.37) | 2-72 | m/z: 734.43(C₅₄H₁₄D₂₂N₂ = 735.03) |
| 2-63 | m/z = 735.01(C₅₄H₁₄D₂₂N₂ = 734.43) | 2-64 | m/z = 735.01(C₅₄H₁₄D₂₂N₂ = 734.43) |
| 2-65 | m/z = 730.98(C₅₄H₁₄D₂₀N₂ = 730.40) | 2-66 | m/z = 735.01(C₅₄H₁₄D₂₂N₂ = 734.43) |
| 2-67 | m/z = 735.01(C₅₄H₁₄D₂₂N₂ = 734.43) | 2-68 | m/z = 815.13(C₆₀H₁₄D₂₆N₂ = 814.48) |
| 2-69 | m/z = 815.13(C₆₀H₁₄D₂₆N₂ = 814.48) | 2-70 | m/z = 815.13(C₆₀H₁₄D₂₆N₂ = 814.48) |
| 2-71 | m/z = 815.13(C₆₀H₁₄D₂₆N₂ = 814.48) | 2-72 | m/z = 666.86(C₄₈H₁₄D₁₆N₂O = 666.34) |
| 2-73 | m/z = 498.68(C₃₆H₁₀D₁₄N₂ = 498.28) | 2-74 | m/z = 650.87(C₄₈H₁₈D₁₄N₂ = 650.34) |
| 2-75 | m/z = 574.77(C₄₁H₁₄D₁₄N₂ = 574.31) | 2-76 | m/z = 648.85(C₄₈H₁₆D₁₄N₂ = 648.33) |
| 2-77 | m/z = 650.87(C₄₈H₁₈D₁₄N₂ = 650.34) | 2-78 | m/z = 650.87(C₄₈H₁₈D₁₄N₂ = 650.34) |
| 2-79 | m/z = 650.87(C₄₈H₁₈D₁₄N₂ = 650.34) | 2-80 | m/z = 650.87(C₄₈H₁₈D₁₄N₂ = 650.34) |
| 2-81 | m/z = 650.87(C₄₈H₁₈D₁₄N₂ = 650.34) | 2-82 | m/z = 726.38(C₅₄H₂₂D₁₄N₂ = 726.98) |
| 2-83 | m/z = 726.96(C₅₄H₂₂D₁₄N₂ = 726.38) | 2-84 | m/z = 726.96(C₅₄H₂₂D₁₄N₂ = 726.38) |
| 2-85 | m/z = 724.95(C₅₄H₂₀D₁₄N₂ = 724.36) | 2-86 | m/z = 726.96(C₅₄H₂₂D₁₄N₂ = 726.38) |
| 2-87 | m/z = 726.96(C₅₄H₂₂D₁₄N₂ = 726.38) | 2-88 | m/z = 803.06(C₆₀H₂₆D₁₄N₂ = 802.41) |
| 2-89 | m/z = 803.06(C₆₀H₂₆D₁₄N₂ = 802.41) | 2-90 | m/z = 803.06(C₆₀H₂₆D₁₄N₂ = 802.41) |
| 2-91 | m/z = 803.06(C₆₀H₂₆D₁₄N₂ = 802.41) | 2-92 | m/z = 803.06(C₆₀H₂₆D₁₄N₂ = 802.41) |
| 2-93 | m/z = 508.74(C₃₆D₂₄N₂ = 508.34) | 2-94 | m/z = 668.98(C₄₈D₃₂N₂ = 668.46) |
| 2-95 | m/z = 588.86(C₄₂D₂₈N₂ = 588.40) | 2-96 | m/z = 664.95(C₄₈D₃₀N₂ = 664.43) |
| 2-97 | m/z = 668.98(C₄₈D₃₂N₂ = 668.46) | 2-98 | m/z = 668.98(C₄₈D₃₂N₂ = 668.46) |
| 2-99 | m/z = 668.98(C₄₈D₃₂N₂ = 668.46) | 2-100 | m/z = 668.98(C₄₈D₃₂N₂ = 668.46) |
| 2-101 | m/z = 668.98(C₄₈D₃₂N₂ = 668.46) | 2-102 | m/z = 748.518(C₅₄D₃₆N₂ = 749.12) |
| 2-103 | m/z = 749.10(C₅₄D₃₆N₂ = 748.51) | 2-104 | m/z = 749.10(C₅₄D₃₆N₂ = 748.51) |
| 2-105 | m/z = 745.07(C₅₄D₃₄N₂ = 744.49) | 2-106 | m/z = 749.10(C₅₄D₃₆N₂ = 748.51) |
| 2-107 | m/z = 749.10(C₅₄D₃₆N₂ = 748.51) | 2-108 | m/z = 829.22(C₆₀D₄₀N₂ = 828.57) |
| 2-109 | m/z = 829.22(C₆₀D₄₀N₂ = 828.57) | 2-110 | m/z = 829.22(C₆₀D₄₀N₂ = 828.57) |
| 2-111 | m/z = 829.22(C₆₀D₄₀N₂ = 828.57) | 2-112 | m/z = 680.95(C₄₈D₃₀N₂O = 680.42) |
| 2-113 | m/z = 697.02(C₄₈D₃₀N₂S = 696.40) | 2-114 | m/z = 829.22(C₆₀D₄₀N₂ = 828.57) |
| 2-115 | m/z = 632.95(C₄₅D₃₂N₂ = 632.46) | 2-116 | m/z = 713.07(C₅₁D₃₆N₂ = 712.51) |

### [Preparation Example 5] Preparation of Compound 3-1 and the Like

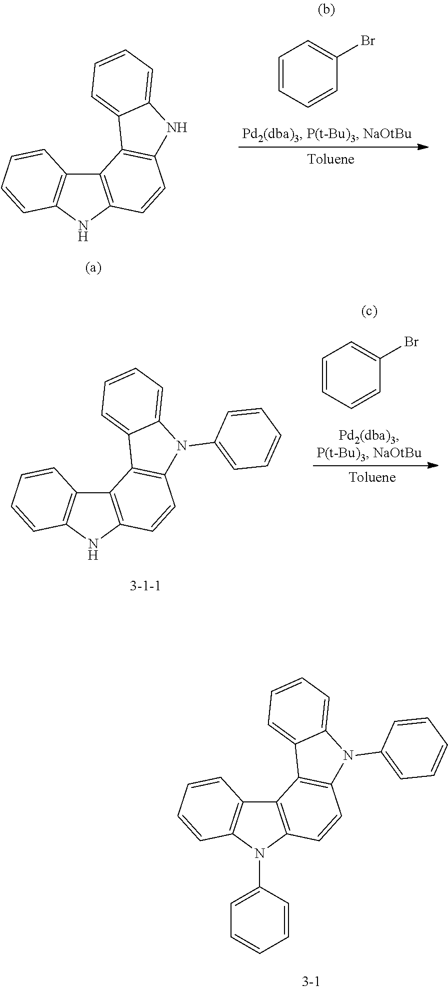 [SMILES: c1ccc2c(c1)[nH]c1ccc3[nH]c4ccccc4c3c12 ; Brc1ccccc1 ; c1ccc(-n2c3ccccc3c3c4c(ccc32)[nH]c2ccccc24)cc1 ; Brc1ccccc1 ; c1ccc(-n2c3ccccc3c3c4c5ccccc5n(-c5ccccc5)c4ccc32)cc1]

### 1) Preparation of Compound 3-1-1

After 10 g (39.0 mmol) of (a) 5,8-dihydroindolo[2,3-c]carbazole, 6.12 g (39.0 mmol) of (b) 1-bromobenzene, 1.79 g (1.95 mmol) of tris(dibenzylideneacetone)dipalladium (Pd₂(dba)₃), 0.92 mL (3.9 mmol) of tri-tert-butylphosphine (P(t-Bu)₃), and 7.50 g (78.0 mmol) of sodium tert-butoxide (NatOBu) were put into a reaction flask, 100 mL of toluene was added thereto, and the resulting mixture was heated at 135° C. for 15 hours. When the reaction was terminated, the resulting product was extracted with methylene chloride (M C) and water, and then purified by column chromatography to obtain 7.3 g of Compound 3-1-1 (yield 56%).

### 2) Preparation of Compound 3-1

After 7.3 g (22.0 mmol) of Compound 3-1-1, (b) 3.8 g (24.2 mmol) of 1-bromobenzene, 1.01 g (1.1 mmol) of tris(dibenzylideneacetone)dipalladium (Pd₂(dba)₃), 0.52 mL (3.9 mmol) of tri-tert-butylphosphine (P(t-Bu)₃), and 4.23 g (44.0 mmol) of sodium tert-butoxide (NatOBu) were put into a reaction flask, 70 mL of toluene was added thereto, and the resulting mixture was heated at 135° C. for 15 hours. When the reaction was terminated, the resulting product was extracted with methylene chloride (M C) and water, and then purified by column chromatography to obtain 8.3 g (yield 93%) of Compound 3-1.

The target compounds in the following Table 9 were synthesized in the same manner as in the preparation of Preparation Example 5, except that Intermediate A in the following Table 9 was used instead of (a), Intermediate B in the following Table 9 was used instead of (b), and Intermediate C in the following Table 9 was used instead of (c) in Preparation Example 5.

TABLE 9

| Compound | | | | |
|---|---|---|---|---|
| No. | Intermediate A | Intermediate B | Intermediate C | Target Compound |
| 3-4 | 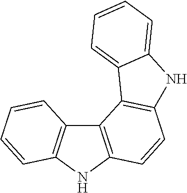 [SMILES: c1ccc2c(c1)[nH]c1ccc3[nH]c4ccccc4c3c12] | 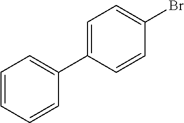 [SMILES: Brc1ccc(-c2ccccc2)cc1] | 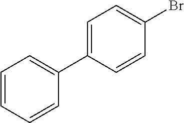 [SMILES: Brc1ccc(-c2ccccc2)cc1] | 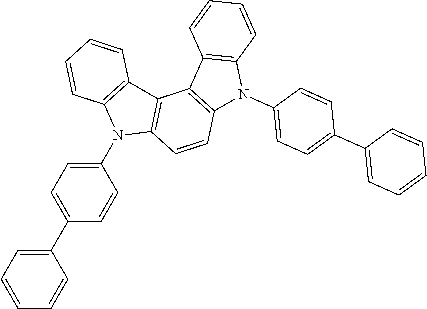 [SMILES: c1ccc(-c2ccc(-n3c4ccccc4c4c5c6ccccc6n(-c6ccc(-c7ccccc7)cc6)c5ccc43)cc2)cc1] |
| 3-5 | 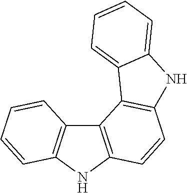 [SMILES: c1ccc2c(c1)[nH]c1ccc3[nH]c4ccccc4c3c12] | 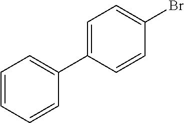 [SMILES: Brc1ccc(-c2ccccc2)cc1] | 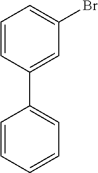 [SMILES: Brc1cccc(-c2ccccc2)c1] | 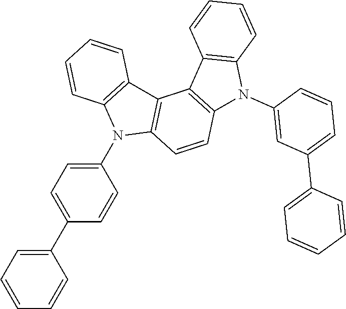 [SMILES: c1ccc(-c2ccc(-n3c4ccccc4c4c5c6ccccc6n(-c6cccc(-c7ccccc7)c6)c5ccc43)cc2)cc1] |
| 3-22 | 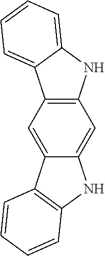 [SMILES: c1ccc2c(c1)[nH]c1cc3[nH]c4ccccc4c3cc12] | 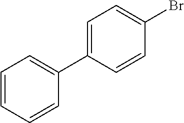 [SMILES: Brc1ccc(-c2ccccc2)cc1] | 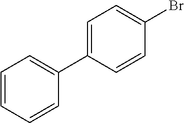 [SMILES: Brc1ccc(-c2ccccc2)cc1] | 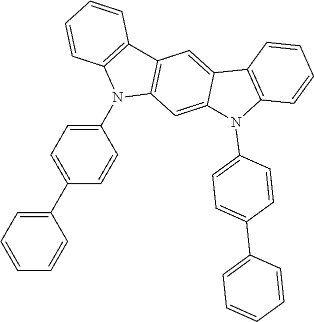 [SMILES: c1ccc(-c2ccc(-n3c4ccccc4c4cc5c6ccccc6n(-c6ccc(-c7ccccc7)cc6)c5cc43)cc2)cc1] |
| 3-23 | 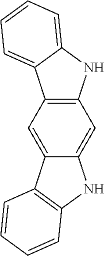 [SMILES: c1ccc2c(c1)[nH]c1cc3[nH]c4ccccc4c3cc12] | 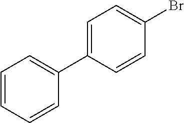 [SMILES: Brc1ccc(-c2ccccc2)cc1] | 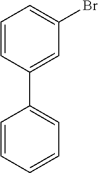 [SMILES: Brc1cccc(-c2ccccc2)c1] | 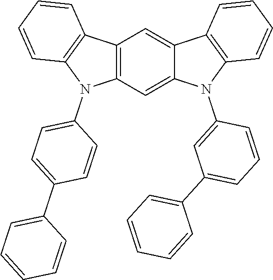 [SMILES: c1ccc(-c2ccc(-n3c4ccccc4c4cc5c6ccccc6n(-c6cccc(-c7ccccc7)c6)c5cc43)cc2)cc1] |
| 3-32 | 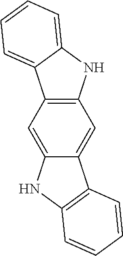 [SMILES: c1ccc2c(c1)[nH]c1cc3c(cc12)[nH]c1ccccc13] | 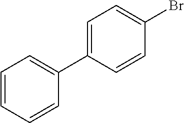 [SMILES: Brc1ccc(-c2ccccc2)cc1] | 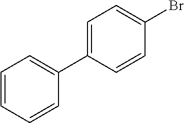 [SMILES: Brc1ccc(-c2ccccc2)cc1] | 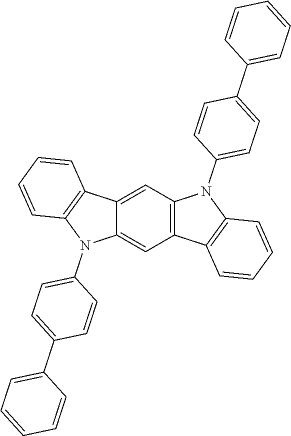 [SMILES: c1ccc(-c2ccc(-n3c4ccccc4c4cc5c(cc43)c3ccccc3n5-c3ccc(-c4ccccc4)cc3)cc2)cc1] |
| 3-35 | 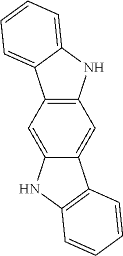 [SMILES: c1ccc2c(c1)[nH]c1cc3c(cc12)[nH]c1ccccc13] | 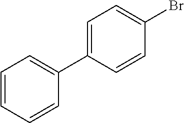 [SMILES: Brc1ccc(-c2ccccc2)cc1] | 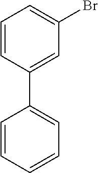 [SMILES: Brc1cccc(-c2ccccc2)c1] | 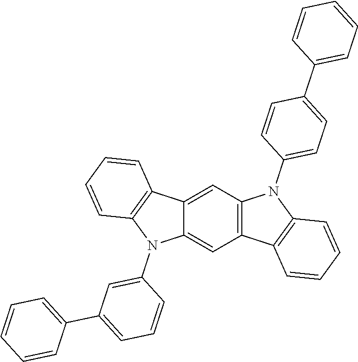 [SMILES: c1ccc(-c2ccc(-n3c4ccccc4c4cc5c(cc43)c3ccccc3n5-c3cccc(-c4ccccc4)c3)cc2)cc1] |
| 3-41 | 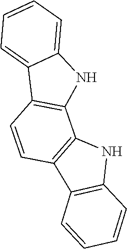 [SMILES: c1ccc2c(c1)[nH]c1c2ccc2c3ccccc3[nH]c21] | 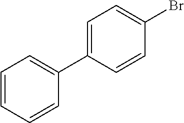 [SMILES: Brc1ccc(-c2ccccc2)cc1] | 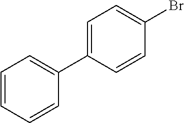 [SMILES: Brc1ccc(-c2ccccc2)cc1] | 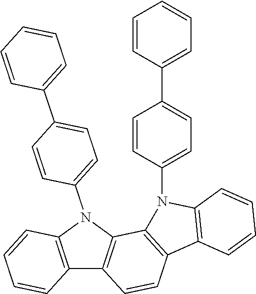 [SMILES: c1ccc(-c2ccc(-n3c4ccccc4c4ccc5c6ccccc6n(-c6ccc(-c7ccccc7)cc6)c5c43)cc2)cc1] |
| 3-61 | 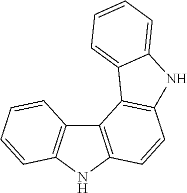 [SMILES: c1ccc2c(c1)[nH]c1ccc3[nH]c4ccccc4c3c12] | 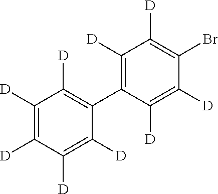 [SMILES: [2H]c1c([2H])c([2H])c(-c2c([2H])c([2H])c(Br)c([2H])c2[2H])c([2H])c1[2H]] | 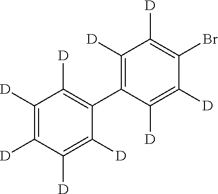 [SMILES: [2H]c1c([2H])c([2H])c(-c2c([2H])c([2H])c(Br)c([2H])c2[2H])c([2H])c1[2H]] | 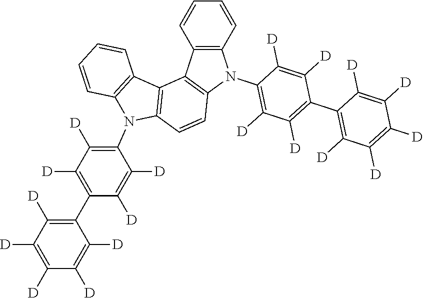 [SMILES: [2H]c1c([2H])c([2H])c(-c2c([2H])c([2H])c(-n3c4ccccc4c4c5c6ccccc6n(-c6c([2H])c([2H])c(-c7c([2H])c([2H])c([2H])c([2H])c7[2H])c([2H])c6[2H])c5ccc43)c([2H])c2[2H])c([2H])c1[2H]] |
| 3-69 | 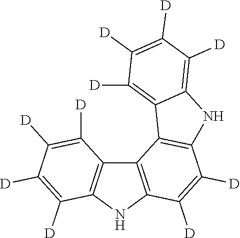 [SMILES: [2H]c1c([2H])c([2H])c2c([nH]c3c([2H])c([2H])c4[nH]c5c([2H])c([2H])c([2H])c([2H])c5c4c32)c1[2H]] | 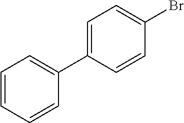 [SMILES: Brc1ccc(-c2ccccc2)cc1] | 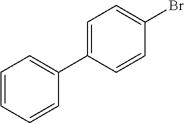 [SMILES: Brc1ccc(-c2ccccc2)cc1] | 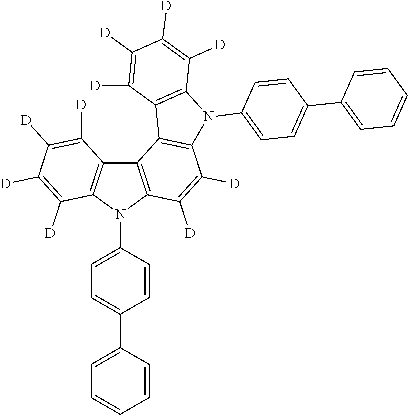 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1] |
| 3-77 | 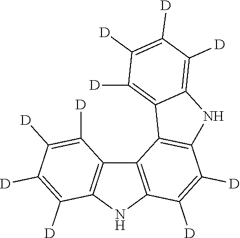 [SMILES: [2H]c1c([2H])c([2H])c2c([nH]c3c([2H])c([2H])c4[nH]c5c([2H])c([2H])c([2H])c([2H])c5c4c32)c1[2H]] | 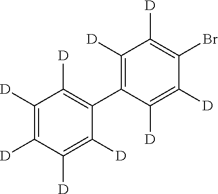 [SMILES: [2H]c1c([2H])c([2H])c(-c2c([2H])c([2H])c(Br)c([2H])c2[2H])c([2H])c1[2H]] | 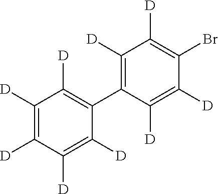 [SMILES: [2H]c1c([2H])c([2H])c(-c2c([2H])c([2H])c(Br)c([2H])c2[2H])c([2H])c1[2H]] | 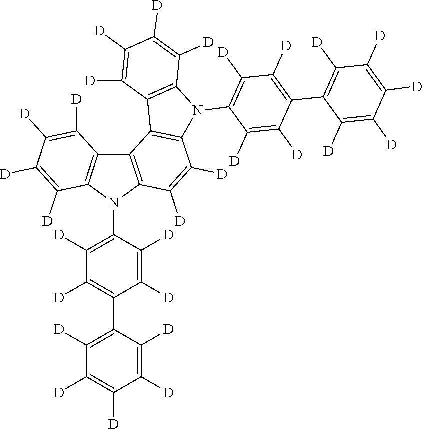 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]] |
| 3-96 | 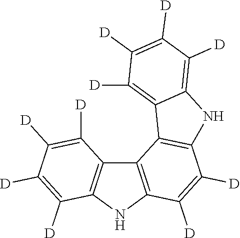 [SMILES: [2H]c1c([2H])c([2H])c2c([nH]c3c([2H])c([2H])c4[nH]c5c([2H])c([2H])c([2H])c([2H])c5c4c32)c1[2H]] | 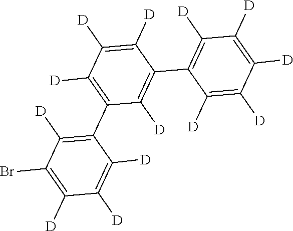 [SMILES: [2H]c1c([2H])c([2H])c(-c2c([2H])c([2H])c([2H])c(-c3c([2H])c([2H])c([2H])c(Br)c3[2H])c2[2H])c([2H])c1[2H]] | 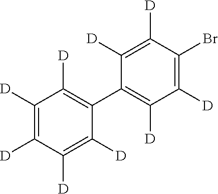 [SMILES: [2H]c1c([2H])c([2H])c(-c2c([2H])c([2H])c(Br)c([2H])c2[2H])c([2H])c1[2H]] | 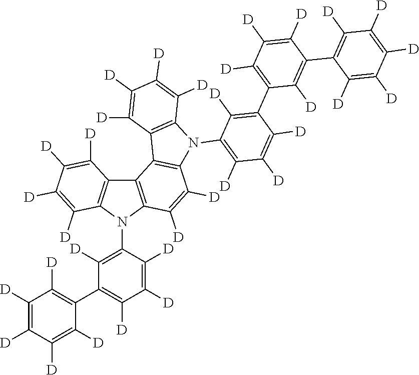 [SMILES: [2H]c1c([2H])c([2H])c(-c2c([2H])c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c([2H])c([2H])c8[2H])c7[2H])c6c([2H])c([2H])c54)c3[2H])c2[2H])c([2H])c1[2H]] |
| 3-99 | 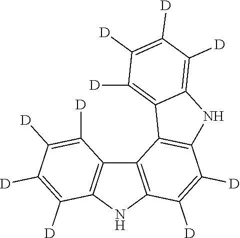 [SMILES: [2H]c1c([2H])c([2H])c2c([nH]c3c([2H])c([2H])c4[nH]c5c([2H])c([2H])c([2H])c([2H])c5c4c32)c1[2H]] | 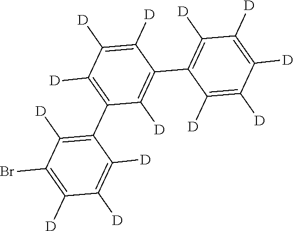 [SMILES: [2H]c1c([2H])c([2H])c(-c2c([2H])c([2H])c([2H])c(-c3c([2H])c([2H])c([2H])c(Br)c3[2H])c2[2H])c([2H])c1[2H]] | 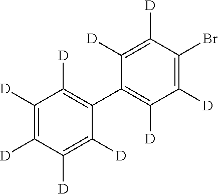 [SMILES: [2H]c1c([2H])c([2H])c(-c2c([2H])c([2H])c(Br)c([2H])c2[2H])c([2H])c1[2H]] | 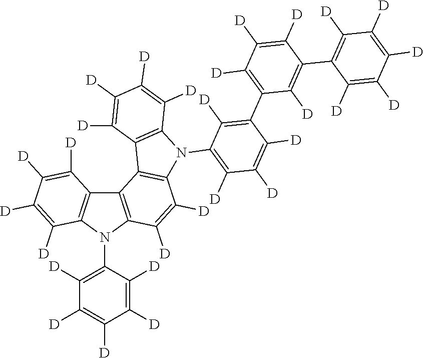 [SMILES: [2H]c1c([2H])c([2H])c(-c2c([2H])c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c3[2H])c2[2H])c([2H])c1[2H]] |

Compounds were prepared in the same manner as in the Preparation Examples, and the synthesis confirmation results thereof are shown in the following Tables 10 and 11. Table 10 shows measured values of field desorption mass spectrometry (FD-MS), and Table 11 shows measured values of ¹H NMR (CDCl₃, 400 MHz).

TABLE 10

| Compound | | Compound | |
|---|---|---|---|
| No. | FD-MS | No. | FD-MS |
| 3-1 | m/z = 408.16(C₃₀H₂₀N₂ = 408.50) | 3-4 | m/z = 560.23(C₄₂H₂₈N₂ = 560.70) |
| 3-5 | m/z = 560.23(C₄₂H₂₈N₂ = 560.70) | 3-22 | m/z = 560.23(C₄₂H₂₈N₂ = 560.70) |
| 3-23 | m/z = 560.23(C₄₂H₂₈N₂ = 560.70) | 3-32 | m/z = 560.23(C₄₂H₂₈N₂ = 560.70) |
| 3-35 | m/z = 560.23(C₄₂H₂₈N₂ = 560.70) | 3-41 | m/z = 560.23(C₄₂H₂₈N₂ = 560.70) |
| 3-61 | m/z = 578.34(C₄₂H₁₀D₁₈N₂ = 578.81) | 3-69 | m/z = 570.29(C₄₂H₁₈D₁₀N₂ = 570.76) |
| 3-77 | m/z = 588.40(C₄₂D₂₈N₂ = 588.87) | 3-96 | m/z = 668.46 (C₄₈D₃₂N₂, 668.99) |
| 3-99 | m/z = 588.40 (C₄₂D₂₈N₂, 588.87) | | |

TABLE 11

| Compound | |
|---|---|
| No. | ¹H NMR(CDCl₃, 400 MHz) |
| 3-1 | δ = 8.55(2H, d), 7.94(2H, d), 7.62-7.35(14H, m), 7.16(2H, d) |
| 3-4 | δ = 8.55(2H, d), 7.94-7.91(10H, m), 7.75(4H, d), 7.49-7.35(10H, m), 7.16(2H, t) |
| 3-5 | δ = 8.55(2H, d), 8.21(1H, s), 7.94-7.91(6H, m), 7.75-7.35(16H, m), 7.26(1H, d), |
| | 7.16(2H, t) |
| 3-22 | δ = 8.55(1H, d), 8.19(1H, d), 7.94-7.91(9H, m), 7.75(4H, d), 7.58-7.35(11H, m), |
| | 7.20-7.16(2H, m) |
| 3-23 | δ = 8.55(1H, d), 8.21-8.19(2H, m), 7.94-7.91(5H, m), 7.75-7.35(18H, m), 7.20- |
| | 7.16(2H, m) |
| 3-32 | δ = 8.55(1H, d), 8.19(1H, d), 7.94-7.91(9H, m), 7.75(4H, d), 7.58-7.35(11H, m), |
| | 7.20-7.16(2H, m) |
| 3-35 | δ = 8.55(1H, d), 8.21-8.19(2H, m), 7.94-7.91(5H, m), 7.68-7.35(18H, m), 7.20- |
| | 7.16(2H, m) |
| 3-41 | δ = 8.55(2H, d), 7.94-7.91(10H, m), 7.84(2H, d), 7.75(4H, d), 7.49-7.35(8H, m), |
| | 7.16(2H, t) |
| 3-61 | δ = 8.55(2H, d), 7.94(2H, d), 7.42-7.35(4H, m), 7.16(2H, t) |
| 3-69 | δ = 7.92-7.91(8H, m), 7.75(4H, d), 7.49-7.41(6H, m) |

### EXPERIMENTAL EXAMPLES

### Experimental Example 1

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-d1(1-naphthyl)-N,N′-diphenyl-(1,1′-biphenyl)-4,4′-diamine (NPB) were formed on the ITO transparent electrode (positive electrode).

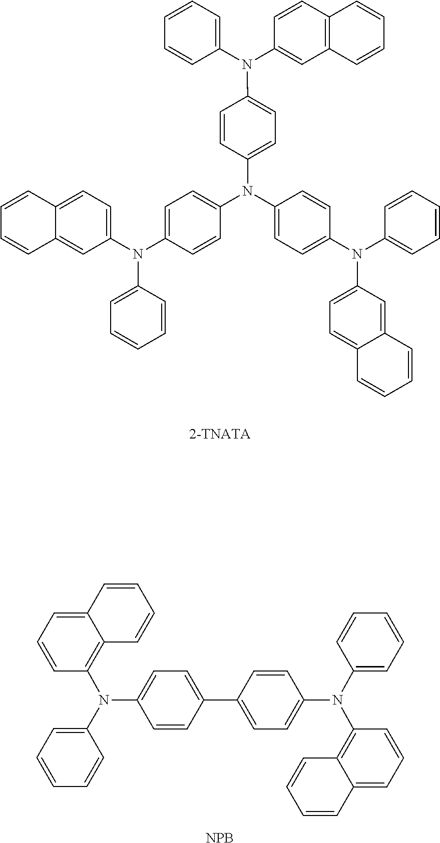 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited to have a thickness of 360 Å by using a compound described in the following Table 12 as a host and tris(2-phenylpyridine)iridium (Ir(ppy)₃) as a green phosphorescent dopant to dope the host with Ir(ppy)₃ in an amount of 6%. Thereafter, 3-benzidino-6-(4-chlorophenyl) pyridazine (BCP) was deposited as a hole blocking layer to have a thickness of 70 Å, and Alq₃ was deposited thereon as an electron transport layer to have a thickness of 250 Å thereon. Finally, lithium fluoride (LiF) was deposited to have a thickness of 10 Å on the electron transport layer to form an electron injection layer, and then an aluminum (Al) negative electrode was deposited to have a thickness of 1200 Å on the electron injection layer to form a negative electrode, thereby manufacturing an organic light emitting device.

The compounds used in the Examples and Comparative Examples in the following Table 12 were used as green hosts. Meanwhile, all the organic compounds required for manufacturing an OLED were subjected to vacuum sublimed purification under 10⁻⁸ torr to 10⁻⁶ torr for each material, and used for the manufacture of OLED.

### [Comparative Compounds]

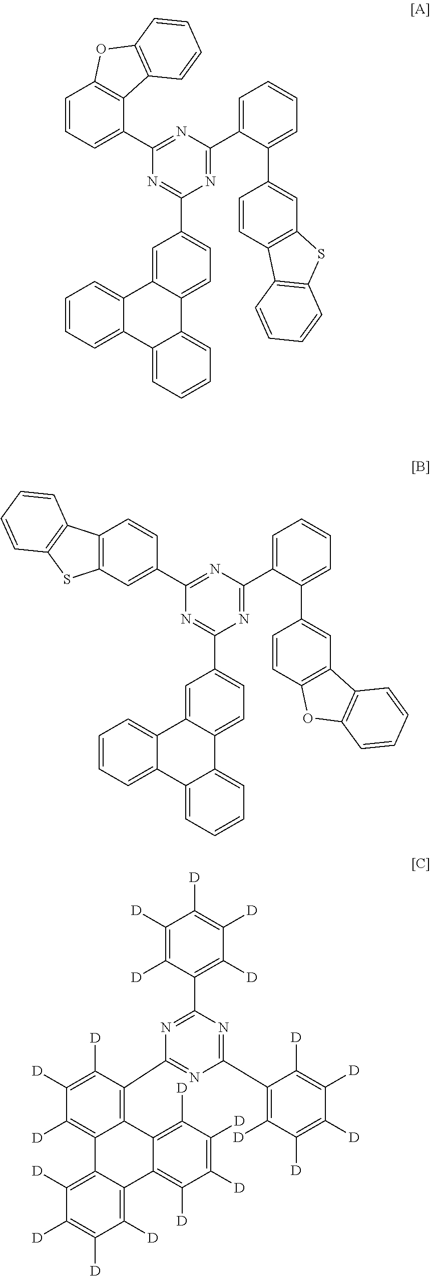

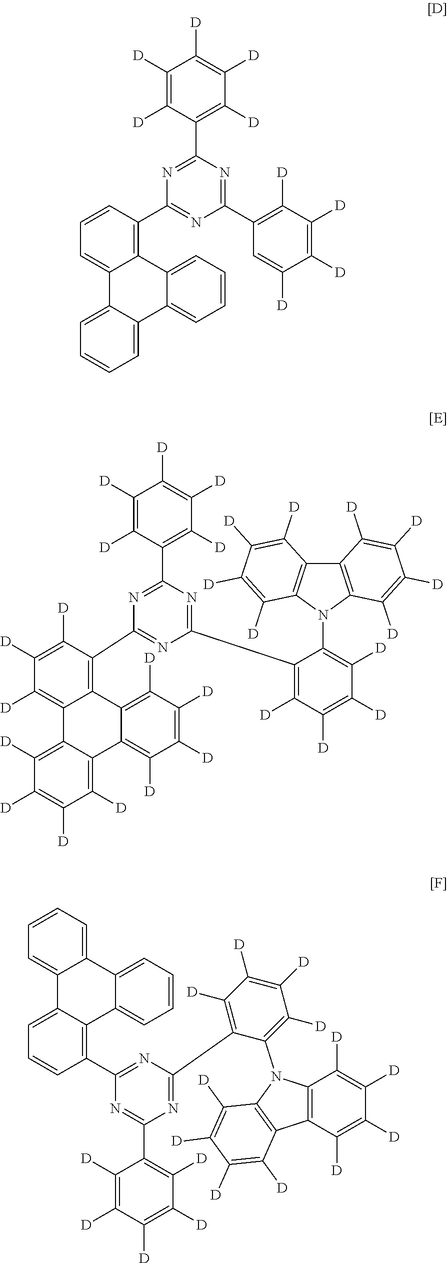

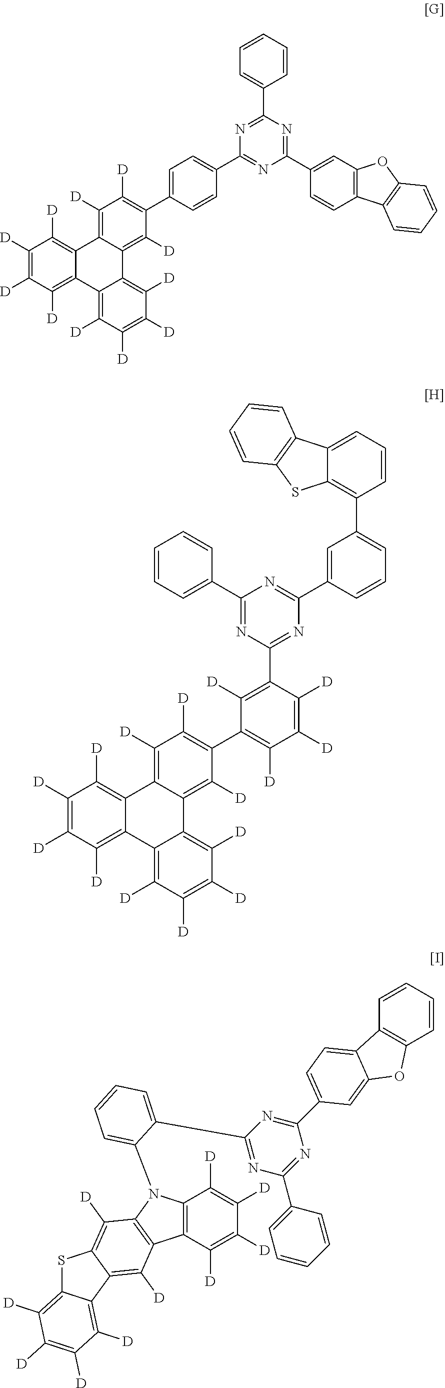 [SMILES: [2H]c1c([2H])c([2H])c2c(c1[2H])c1c([2H])c([2H])c([2H])c([2H])c1c1c([2H])c(-c3ccc(-c4nc(-c5ccccc5)nc(-c5ccc6c(c5)oc5ccccc56)n4)cc3)c([2H])c([2H])c21 ; [2H]c1c([2H])c(-c2nc(-c3ccccc3)nc(-c3cccc(-c4cccc5c4sc4ccccc45)c3)n2)c([2H])c(-c2c([2H])c([2H])c3c4c([2H])c([2H])c([2H])c([2H])c4c4c([2H])c([2H])c([2H])c([2H])c4c3c2[2H])c1[2H] ; [2H]c1c([2H])c([2H])c2c(sc3c([2H])c4c(c([2H])c32)c2c([2H])c([2H])c([2H])c([2H])c2n4-c2ccccc2-c2nc(-c3ccccc3)nc(-c3ccc4c(c3)oc3ccccc34)n2)c1[2H]]

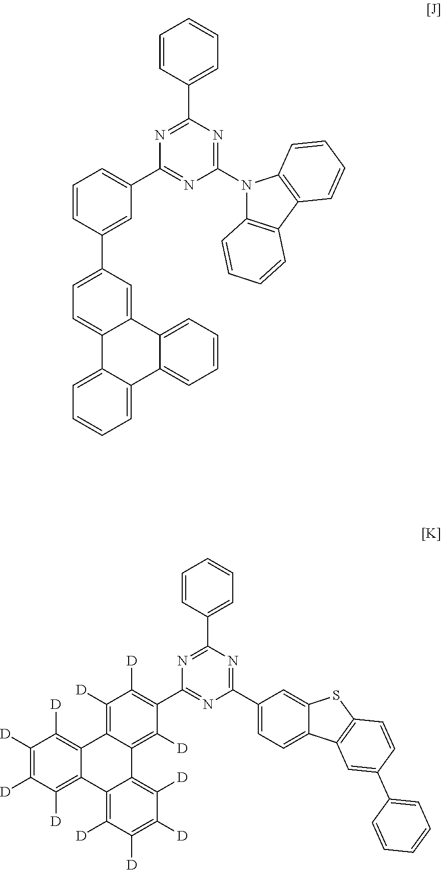 [SMILES: c1ccc(-c2nc(-c3cccc(-c4ccc5c6ccccc6c6ccccc6c5c4)c3)nc(-n3c4ccccc4c4ccccc43)n2)cc1 ; [2H]c1c([2H])c([2H])c2c(c1[2H])c1c([2H])c([2H])c([2H])c([2H])c1c1c([2H])c(-c3nc(-c4ccccc4)nc(-c4ccc5c(c4)sc4ccc(-c6ccccc6)cc45)n3)c([2H])c([2H])c21]

### 2) Driving Voltage and Light Emitting Efficiency of Organic Light Emitting Device

For the organic light emitting device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 12,000 cd/m².

The results of measuring the driving voltage, light emitting efficiency, EL color, and service life of the organic light emitting device manufactured according to the present invention are shown in the following Table 12. T₉₀ means the service life (unit: hour) that is the time it takes for the luminance to reach 90% relative to the initial luminance.

TABLE 12

| | Compound | | Light | | |
|---|---|---|---|---|---|
| | (light | Driving | emitting | | Service |
| | emitting | voltage | efficiency | | life |
| No. | material) | (V) | (cd/A) | EL color | (T₉₀) |
| Example 1 | 1 | 3.67 | 106.7 | Green | 102 |
| Example 2 | 4 | 3.71 | 106.9 | | 105 |
| Example 3 | 5 | 3.78 | 104.8 | | 89 |
| Example 4 | 7 | 3.71 | 105.7 | | 95 |
| Example 5 | 10 | 3.66 | 105.3 | | 98 |
| Example 6 | 12 | 3.69 | 105.6 | | 88 |
| Example 7 | 14 | 3.78 | 105.3 | | 89 |
| Example 8 | 18 | 3.76 | 104.8 | | 92 |
| Example 9 | 23 | 3.74 | 105.5 | | 97 |
| Example 10 | 29 | 3.72 | 105.0 | | 91 |
| Example 11 | 37 | 3.76 | 107.1 | | 104 |
| Example 12 | 39 | 3.77 | 104.9 | | 95 |
| Example 13 | 42 | 3.71 | 104.8 | | 89 |
| Example 14 | 44 | 3.78 | 104.8 | | 86 |
| Example 15 | 56 | 3.74 | 106.2 | | 94 |
| Example 16 | 61 | 3.73 | 106.3 | | 87 |
| Example 17 | 63 | 3.77 | 105.6 | | 89 |
| Example 18 | 70 | 3.72 | 105.3 | | 92 |
| Example 19 | 73 | 3.65 | 105.8 | | 87 |
| Example 20 | 76 | 3.75 | 105.9 | | 88 |
| Example 21 | 77 | 3.72 | 105.6 | | 98 |
| Example 22 | 84 | 3.68 | 104.8 | | 96 |
| Example 23 | 86 | 3.71 | 105.4 | | 98 |
| Example 24 | 93 | 3.68 | 105.7 | | 88 |
| Example 25 | 98 | 3.73 | 106.1 | | 93 |
| Example 26 | 105 | 3.70 | 106.9 | | 106 |
| Example 27 | 112 | 3.70 | 105.9 | | 90 |
| Example 28 | 115 | 3.66 | 105.2 | | 95 |
| Example 29 | 121 | 3.67 | 106.8 | | 104 |
| Example 30 | 128 | 3.74 | 105.4 | | 90 |
| Example 31 | 130 | 3.70 | 106.0 | | 96 |
| Example 32 | 149 | 3.71 | 106.0 | | 87 |
| Example 33 | 157 | 3.75 | 107.0 | | 103 |
| Example 34 | 207 | 3.75 | 105.0 | | 87 |
| Example 35 | 217 | 3.66 | 105.8 | | 93 |
| Example 36 | 220 | 3.67 | 106.1 | | 93 |
| Example 37 | 224 | 3.66 | 106.9 | | 102 |
| Example 38 | 228 | 3.74 | 105.8 | | 92 |
| Example 39 | 231 | 3.66 | 106.1 | | 91 |
| Example 40 | 233 | 3.77 | 105.3 | | 94 |
| Example 41 | 235 | 3.75 | 106.7 | | 101 |
| Example 42 | 237 | 3.71 | 106.5 | | 100 |
| Example 43 | 242 | 3.66 | 105.2 | | 97 |
| Example 44 | 244 | 3.77 | 105.5 | | 95 |
| Example 45 | 251 | 3.70 | 105.8 | | 95 |
| Example 46 | 275 | 3.68 | 106.7 | | 99 |
| Example 47 | 282 | 3.67 | 106.2 | | 89 |
| Example 48 | 289 | 3.70 | 105.6 | | 90 |
| Example 49 | 295 | 3.73 | 106.1 | | 89 |
| Example 50 | 312 | 3.68 | 106.2 | | 88 |
| Example 51 | 331 | 3.66 | 105.7 | | 93 |
| Example 52 | 361 | 3.75 | 105.8 | | 88 |
| Example 53 | 381 | 3.76 | 106.2 | | 92 |
| Example 54 | 386 | 3.72 | 105.2 | | 87 |
| Example 55 | 406 | 3.75 | 106.0 | | 90 |
| Example 56 | 415 | 3.73 | 105.7 | | 94 |
| Example 57 | 427 | 3.75 | 105.6 | | 93 |
| Example 58 | 439 | 3.74 | 105.1 | | 94 |
| Example 59 | 455 | 3.75 | 105.3 | | 91 |
| Example 60 | 464 | 3.71 | 105.3 | | 95 |
| Example 61 | 475 | 3.70 | 105.6 | | 92 |
| Example 62 | 503 | 3.77 | 104.1 | | 83 |
| Example 63 | 511 | 3.74 | 104.3 | | 86 |
| Example 64 | 516 | 3.76 | 103.6 | | 84 |
| Comparative | A | 3.88 | 100.9 | | 75 |
| Example 1 | | | | | |
| Comparative | B | 3.92 | 99.3 | | 72 |
| Example 2 | | | | | |
| Comparative | C | 3.93 | 91.2 | | 63 |
| Example 3 | | | | | |
| Comparative | D | 3.91 | 89.7 | | 52 |
| Example 4 | | | | | |
| Comparative | E | 3.82 | 101.3 | | 74 |
| Example 5 | | | | | |
| Comparative | F | 3.83 | 101.8 | | 72 |
| Example 6 | | | | | |
| Comparative | G | 3.94 | 100.5 | | 71 |
| Example 7 | | | | | |
| Comparative | H | 3.85 | 100.7 | | 75 |
| Example 8 | | | | | |
| Comparative | I | 3.96 | 87.5 | | 61 |
| Example 9 | | | | | |
| Comparative | J | 3.85 | 98.8 | | 58 |
| Example 10 | | | | | |
| Comparative | K | 3.86 | 99.3 | | 75 |
| Example 11 | | | | | |

When the results in Table 12 are observed, the organic light emitting devices of Examples 1 to 64 including the heterocyclic compound represented by Chemical Formula 1 of the present invention had a driving voltage decreased by up to 7.83%, a light emitting efficiency increased by up to 22.40%, and a service life increased by up to 82.76% compared to Comparative Examples 1 to 11.

In the case of the structure of the heterocyclic compound satisfying the structural features of Chemical Formula 1 of the present invention, the triphenylene and azine-based substituents are bonded to increase the rigidity of the structure, thereby enhancing the thermal stability during material deposition. In addition, it is determined that the substitution of one or more carbon atoms of the triphenylene (provided that the portion to which the azine-based substituent is linked is excluded) with deuterium serves to strengthen the LUMO. Furthermore, the inclusion of an arylene linker in the triazine reduces the degree of overlap between the HOMO and LUMO orbitals, which improves the stability of the electrons and holes.

In contrast, in the case of Comparative Compounds A, B, and J, it can be confirmed that the device characteristics such as efficiency and service life deteriorated compared to the Examples. The comparative compounds have in common that not only the 11 substitutable carbon atoms of the triphenylene moiety (except for the portion to which the azine-based substituent is linked) but also the entire compound are not substituted with deuterium. Accordingly, it is determined that differences in device characteristics appear because the LUMO is relatively weakened, electrons are slowly injected, and the charge balance is not achieved compared to the structures of the example compounds.

Comparative Compound C has a structure in which the triphenylene moiety and other azine-based substituents are substituted with deuterium, and since not only the LUMO but also the HOMO are strengthened, the hole transfer is relatively fast, the charge balance is not achieved, so that the device characteristics such as efficiency and service life deteriorated. Further, since this structure does not include a linker in the triazine substituent, the degree of distribution and overlap of both the HOMO and LUMO orbitals in the triphenylene and triazine portions is greater than in the structure of the present invention, resulting in a decrease in the stability of electrons and holes and a shortened service life.

In the case of Comparative Compounds D and F, the triphenylene is unsubstituted and the azine-based substituent is substituted with deuterium, so that the triphenylene acts as the LUMO, and the LUMO is weakened compared to the example compounds, resulting in a deterioration in the device characteristics.

As Comparative Example I does not include a triphenylene moiety, it can be confirmed that the hole transfer is relatively fast, and the charge balance is not achieved, resulting in a decrease in efficiency and service life.

In the case of Comparative Compound E, all of the moieties acting as the HOMO and LUMO are substituted with deuterium. Since the HOMO is further strengthened compared to the example compounds, the hole transfer is relatively fast and the charge balance is not achieved, resulting in a decrease in efficiency and service life.

In the case of Comparative Compound G, the LUMO seems to be strengthened, in that all 11 carbon atoms of the triphenylene except for the azine-based substituent are substituted with deuterium, but as any one of the terminal groups of the azine-based substituent does not satisfy the structure including an arylene linker, the LUMO is weakened, so that the device characteristics deteriorate.

In the case of Comparative Compound H, a phenylene group is interposed between the azine-based substituent and its terminal group, and one or more carbon atoms of the triphenylene are substituted with deuterium except for the carbon linked to the azine-based substituent, but a linker (L3) is additionally substituted with deuterium between the triphenylene core and the triazine. As a result, the LUMO cannot be decentralized to the triphenylene by the deuterium-substituted phenylene group linker, and accordingly, the LUMO is weakened, so that the device characteristics deteriorate.

Comparative Compound K has a structure in which the LUMO is strengthened in that triphenylene is directly linked to triazine and deuterium is substituted, and has a structure in which a heteroaryl group is directly bonded to the L2 portion instead of a phenylene group linker and a phenyl group is linked to the terminal group portion, unlike the structure of the present invention. Since the L2 portion does not include a phenylene group linker, the degree of overlap between the HOMO and LUMO increases, and the stability of the electrons and holes decreases, resulting in a shortened service life.

### Experimental Example 2

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4,4″-tris[2-naphthyl(phenyl)amino]triphenylamine (2-TNATA) and the hole transport layer N,N′-d1(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. Pre-mixing was performed by pre-mixing two compounds (N-type host (N) and P-type host (P)) shown in the following Table 13 as hosts at the weight ratio shown in the following Table 13, and then the light emitting layer was deposited to have a thickness of 350 Å in one common container, and deposited by doping the host with Ir(ppy)₃ as a green phosphorescent dopant in an amount of 6% of the deposition thickness of the light emitting layer. Thereafter, BC P was deposited as a hole blocking layer to have a thickness of 70 Å, and Alq₃ was deposited as an electron transport layer to have a thickness of 250 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic light emitting device.

Meanwhile, all the organic compounds required for manufacturing an OLED device were subjected to vacuum sublimed purification under 10⁻⁸ torr to 10⁻⁶ torr for each material, and used for the manufacture of the OLED.

For the organic light emitting device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 12,000 cd/m².

The results of measuring the driving voltage, light emitting efficiency, EL color, and service life (T₉₀) of the organic light emitting device manufactured according to the present invention are shown as in the following Table 13.

TABLE 13

| | Combination | Ratio | | | | |
|---|---|---|---|---|---|---|
| | of light | (N:P) | | Light | | |
| | emitting layer | based on | Driving | emitting | | Service |
| | compounds | weight | voltage | efficiency | | life |
| No. | (N:P) | ratio | (V) | (cd/A) | EL color | (T₉₀) |
| Example 65 | 1:2-1 | 1:1 | 3.74 | 113.9 | Green | 112 |
| Example 66 | | 1:2 | 3.78 | 112.5 | | 114 |
| Example 67 | | 1:3 | 3.81 | 111.9 | | 117 |
| Example 68 | 4:2-93 | 1:1 | 3.75 | 112.1 | | 109 |
| Example 69 | | 1:2 | 3.78 | 111.3 | | 114 |
| Example 70 | | 1:3 | 3.82 | 110.5 | | 116 |
| Example 71 | 37:2-93 | 1:1 | 3.80 | 113.5 | | 110 |
| Example 72 | | 1:2 | 3.84 | 111.7 | | 115 |
| Example 73 | | 1:3 | 3.85 | 110.2 | | 119 |
| Example 74 | 105:2-100 | 1:1 | 3.81 | 114.7 | | 108 |
| Example 75 | | 1:2 | 3.83 | 113.1 | | 112 |
| Example 76 | | 1:3 | 3.85 | 112.0 | | 115 |
| Example 77 | 105:3-14 | 1:1 | 3.78 | 111.6 | | 111 |
| Example 78 | | 1:2 | 3.80 | 109.8 | | 113 |
| Example 79 | | 1:3 | 3.82 | 108.3 | | 116 |
| Example 80 | 121:2-97 | 1:1 | 3.81 | 114.0 | | 108 |
| Example 81 | | 1:2 | 3.82 | 112.7 | | 111 |
| Example 82 | | 1:3 | 3.84 | 111.2 | | 115 |
| Example 83 | 157:2-97 | 1:1 | 3.83 | 114.5 | | 110 |
| Example 84 | | 1:2 | 3.85 | 112.9 | | 112 |
| Example 85 | | 1:3 | 3.89 | 111.6 | | 114 |
| Example 86 | 224:2-100 | 1:1 | 3.82 | 113.0 | | 109 |
| Example 87 | | 1:2 | 3.85 | 112.3 | | 111 |
| Example 88 | | 1:3 | 3.87 | 110.6 | | 117 |
| Example 89 | 241:2-112 | 1:1 | 3.82 | 112.6 | | 109 |
| Example 90 | | 1:2 | 3.84 | 110.7 | | 112 |
| Example 91 | | 1:3 | 3.85 | 109.4 | | 118 |
| Example 92 | 241:3-12 | 1:1 | 3.79 | 109.2 | | 113 |
| Example 93 | | 1:2 | 3.82 | 108.7 | | 115 |
| Example 94 | | 1:3 | 3.86 | 107.5 | | 119 |
| Example 95 | 243:3-14 | 1:1 | 3.82 | 110.3 | | 107 |
| Example 96 | | 1:2 | 3.84 | 109.6 | | 110 |
| Example 97 | | 1:3 | 3.85 | 108.9 | | 114 |
| Example 98 | 243:3-80 | 1:1 | 3.79 | 111.4 | | 112 |
| Example 99 | | 1:2 | 3.82 | 110.0 | | 116 |
| Example 100 | | 1:3 | 3.84 | 109.7 | | 118 |
| Example 101 | 381:3-78 | 1:1 | 3.82 | 110.3 | | 111 |
| Example 102 | | 1:2 | 3.84 | 109.8 | | 113 |
| Example 103 | | 1:3 | 3.85 | 109.1 | | 116 |
| Example 104 | 381:2-114 | 1:1 | 3.79 | 110.5 | | 109 |
| Example 105 | | 1:2 | 3.83 | 110.1 | | 111 |
| Example 106 | | 1:3 | 3.84 | 109.7 | | 115 |
| Example 107 | 415:3-14 | 1:1 | 3.80 | 109.8 | | 108 |
| Example 108 | | 1:2 | 3.83 | 109.6 | | 112 |
| Example 109 | | 1:3 | 3.85 | 109.3 | | 116 |
| Example 110 | 427:2-101 | 1:1 | 3.78 | 109.9 | | 112 |
| Example 111 | | 1:2 | 3.80 | 109.5 | | 115 |
| Example 112 | | 1:3 | 3.81 | 109.2 | | 117 |
| Example 113 | 439:3-14 | 1:1 | 3.82 | 109.6 | | 112 |
| Example 114 | | 1:2 | 3.84 | 109.4 | | 114 |
| Example 115 | | 1:3 | 3.85 | 109.2 | | 115 |
| Example 116 | 503:2-101 | 1:1 | 3.84 | 108.2 | | 103 |
| Example 117 | | 1:2 | 3.86 | 107.8 | | 106 |
| Example 118 | | 1:3 | 3.87 | 107.4 | | 109 |
| Example 119 | 511:3-14 | 1:1 | 3.78 | 108.4 | | 105 |
| Example 120 | | 1:2 | 3.80 | 107.7 | | 108 |
| Example 121 | | 1:3 | 3.81 | 107.4 | | 110 |
| Comparative | A:2-114 | 1:1 | 3.99 | 103.2 | | 80 |
| Example 12 | | | | | | |
| Comparative | | 1:2 | 4.01 | 102.4 | | 83 |
| Example 13 | | | | | | |
| Comparative | | 1:3 | 4.00 | 101.6 | | 85 |
| Example 14 | | | | | | |
| Comparative | B:2-100 | 1:1 | 4.03 | 102.8 | | 78 |
| Example 15 | | | | | | |
| Comparative | | 1:2 | 4.04 | 101.3 | | 80 |
| Example 16 | | | | | | |
| Comparative | | 1:3 | 4.06 | 100.6 | | 81 |
| Example 17 | | | | | | |
| Comparative | C:2-97 | 1:1 | 4.04 | 96.2 | | 70 |
| Example 18 | | | | | | |
| Comparative | | 1:2 | 4.06 | 95.5 | | 74 |
| Example 19 | | | | | | |
| Comparative | | 1:3 | 4.07 | 94.3 | | 76 |
| Example 20 | | | | | | |
| Comparative | D:3-12 | 1:1 | 4.01 | 91.1 | | 67 |
| Example 21 | | | | | | |
| Comparative | | 1:2 | 4.03 | 90.3 | | 70 |
| Example 22 | | | | | | |
| Comparative | | 1:3 | 4.04 | 89.7 | | 72 |
| Example 23 | | | | | | |
| Comparative | E:3-14 | 1:1 | 3.90 | 105.5 | | 90 |
| Example 24 | | | | | | |
| Comparative | | 1:2 | 3.91 | 104.3 | | 93 |
| Example 25 | | | | | | |
| Comparative | | 1:3 | 3.88 | 103.7 | | 94 |
| Example 26 | | | | | | |
| Comparative | F:3-78 | 1:1 | 3.93 | 102.1 | | 89 |
| Example 27 | | | | | | |
| Comparative | | 1:2 | 3.92 | 101.7 | | 91 |
| Example 28 | | | | | | |
| Comparative | | 1:3 | 3.94 | 100.3 | | 93 |
| Example 29 | | | | | | |
| Comparative | G:2-97 | 1:1 | 4.01 | 102.3 | | 89 |
| Example 30 | | | | | | |
| Comparative | | 1:2 | 4.03 | 101.6 | | 92 |
| Example 31 | | | | | | |
| Comparative | | 1:3 | 4.06 | 101.0 | | 93 |
| Example 32 | | | | | | |
| Comparative | H:3-12 | 1:1 | 3.94 | 102.9 | | 90 |
| Example 33 | | | | | | |
| Comparative | | 1:2 | 3.95 | 102.1 | | 93 |
| Example 34 | | | | | | |
| Comparative | | 1:3 | 3.92 | 101.8 | | 94 |
| Example 35 | | | | | | |
| Comparative | I:3-14 | 1:1 | 4.02 | 90.7 | | 70 |
| Example 36 | | | | | | |
| Comparative | | 1:2 | 4.05 | 90.2 | | 73 |
| Example 37 | | | | | | |
| Comparative | | 1:3 | 4.07 | 89.8 | | 74 |
| Example 38 | | | | | | |
| Comparative | J:2-100 | 1:1 | 3.96 | 102.1 | | 67 |
| Example 39 | | | | | | |
| Comparative | | 1:2 | 3.95 | 101.7 | | 71 |
| Example 40 | | | | | | |
| Comparative | | 1:3 | 3.97 | 101.3 | | 73 |
| Example 41 | | | | | | |
| Comparative | K:3-12 | 1:1 | 3.92 | 97.3 | | 85 |
| Example 42 | | | | | | |
| Comparative | | 1:2 | 3.94 | 96.7 | | 87 |
| Example 43 | | | | | | |
| Comparative | | 1:3 | 3.95 | 96.3 | | 90 |
| Example 44 | | | | | | |

Comparing the results of Table 12 with those of Table 13, it can be confirmed that when the heterocyclic compound of Chemical Formula 1 and the compound of Chemical Formula 2 or 3 are used simultaneously as hosts in the light emitting layer, specifically, when the heterocyclic compound of Chemical Formula 1 is used as an N-type host and the compound of Chemical Formula 2 or 3 is used as a P-type host, the driving voltage, light emitting efficiency and/or service life are improved. From these results, it can be expected that an exciplex phenomenon will occur when both compounds are included.

The exciplex phenomenon is a phenomenon in which energy with a magnitude of the HOMO level of a donor (p-host) and the LUMO level of an acceptor (n-host) is released due to an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. In the present invention, it could be confirmed that the heterocyclic compound of Chemical Formula 1 serves as an acceptor and the compound of Chemical Formula 2 and/or 3 serves as a donor, so that when the compounds were used as a host in a light emitting layer, excellent device characteristics were exhibited.

When the results in Table 13 are observed, the organic light emitting devices of Examples 65 to 121 including a combination of the heterocyclic compound represented by Chemical Formula 1 and the heterocyclic compound of Chemical Formula 2 or 3 of the present invention had a driving voltage decreased by up to 8.11%, a light emitting efficiency increased by up to 27.87%, and a service life increased by up to 77.61% compared to Comparative Examples 12 to 44.

In particular, it can be confirmed that when the compound of Chemical Formula 2 or 3 includes deuterium, service life characteristics are further excellent. In Examples 65 to 121 in Table 13, for 15 compounds of Chemical Formula 1, two compounds different in the inclusion of deuterium in the same structure were used in combination as P-type hosts, and from the results in Table 13, it can be seen that when the heterocyclic compound of Chemical Formula 1 is used in combination with the compound of Chemical Formula 2 or 3 including deuterium, the service life is remarkably improved. This is determined that when the compound includes deuterium relative to the same structure, the compound shows much more balanced charge transport characteristics than a compound that does not include deuterium, and the stability of the entire molecule is increased due to the high single bond dissociation energy of carbon and deuterium, thereby increasing the service life.

In contrast, it can be seen that when the compounds out of the scope of the present invention are used as N-type hosts, and are here used in combination with the heterocyclic compound of Chemical Formula 2 or 3 (Comparative Examples 12 to 44), the performance in terms of driving voltage, light emitting efficiency and service life deteriorates compared to the present invention.

That is, it can be confirmed that when the heterocyclic compound of Chemical Formula 1 and the heterocyclic compound of Chemical Formula 2 or 3 of the present invention are simultaneously used as hosts of a light emitting layer, the driving voltage, light emitting efficiency and service life are remarkably excellent.

The present invention is not limited to the Examples, but may be prepared in various forms, and a person with ordinary skill in the art to which the present invention pertains will understand that the present invention can be implemented in another specific form without changing the technical spirit or essential feature of the present invention. Therefore, it should be understood that the above-described Examples are illustrative only in all aspects and are not restrictive.

1. A heterocyclic compound represented by the following Chemical Formula 1: 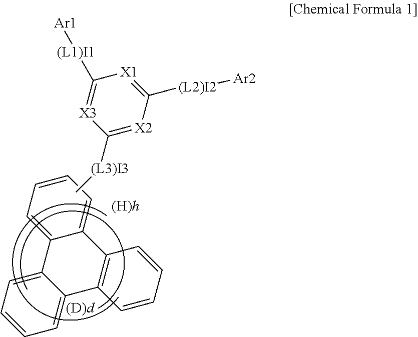
wherein, in Chemical Formula 1,
X1 to X3 are the same as or different from each other, and are each N or CRa, and one or more of X1 to X3 are N,
Ra is hydrogen; a substituted or unsubstituted C6 to C60 aryl group; or a substituted or unsubstituted C2 to C60 heteroaryl group,
L1 to L3 are the same as or different from each other, and are each independently a direct bond; a substituted or unsubstituted C6 to C60 arylene group; or a substituted or unsubstituted C2 to C60 heteroarylene group, and at least one of L1 and L2 is a substituted or unsubstituted C6 to C60 arylene group,
Ar1 and Ar2 are the same as or different from each other, and are each independently a substituted or unsubstituted C6 to C60 aryl group; or a substituted or unsubstituted C2 to C60 heteroaryl group,
I1 to I3 are the same as or different from each other, and are each independently an integer from 1 to 4, and when each of I1 to I3 is an integer of 2 or higher, substituents in the parenthesis are the same as or different from each other,
H is hydrogen,
D is deuterium,
h is an integer from 0 to 10,
d is an integer from 1 to 11, and
a sum of h and d is 11.

2. The heterocyclic compound of claim 1, wherein Chemical Formula 1 is represented by the following Chemical Formula 1-1 or 1-2: 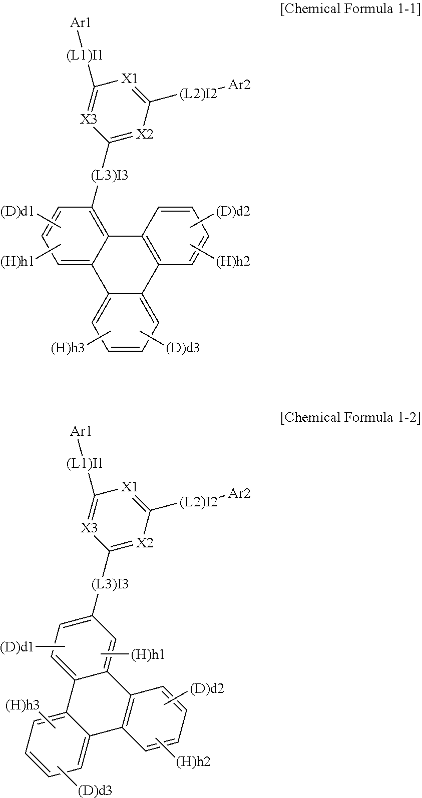
in Chemical Formulae 1-1 and 1-2,
each of X1 to X3, L1 to L3, Ar1, Ar2, 11 to 13, H, and D is the same as that defined in Chemical Formula 1,
h1 is an integer from 0 to 3,
h2 and h3 are each an integer from 0 to 4,
d1 is an integer from 0 to 3,
d2 and d3 are each an integer from 0 to 4,
the sum of h1 and d1 is 3,
the sum of h2 and d2 and the sum of h3 and d3 are each 4, and
the sum of d1, d2, and d3 are an integer from 1 to 11.

3. The heterocyclic compound of claim 1, wherein “substituted or unsubstituted” means being unsubstituted or substituted with one or more substituents selected from the group consisting of a C1 to C60 alkyl group; a C3 to C60 cycloalkyl group; a C2 to C60 heterocycloalkyl group; a C6 to C60 aryl group; a C2 to C60 heteroaryl group; —SiR′R″R′″; and —P(═O)R′R″, or being unsubstituted or substituted with a substituent to which two or more substituents selected from the above substituents are linked, or means that two or more substituents selected from the above substituents are bonded to each other to form a ring, and
R′, R″, and R′″ are the same as or different from each other, and are each independently hydrogen; a C1 to C60 alkyl group; a C3 to C60 cycloalkyl group; a C6 to C60 aryl group; or a C2 to C60 heteroaryl group.

4. The heterocyclic compound of claim 1, wherein a deuterium content of 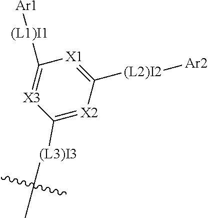
of Chemical Formula 1 is 0%, and 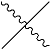
means a linking position in Chemical Formula 1.

5. The heterocyclic compound of claim 1, wherein L1 to L3 are the same as or different from each other, and are each independently a direct bond; a substituted or unsubstituted C6 to C30 arylene group; or a substituted or unsubstituted C2 to C30 heteroarylene group, and at least one of L1 and L2 is a substituted or unsubstituted C6 to C30 arylene group.

6. The heterocyclic compound of claim 1, wherein a deuterium content of the heterocyclic compound represented by Chemical Formula 1 is more than 0% and 41% or less.

7. The heterocyclic compound of claim 1, wherein when L1 is a substituted or unsubstituted C6 to C60 arylene group, Ar1 is a substituted or unsubstituted C2 to C60 heteroaryl group, or
when L2 is a substituted or unsubstituted C6 to C60 arylene group, Ar2 is a substituted or unsubstituted C2 to C60 heteroaryl group.

8. The heterocyclic compound of claim 1, wherein the heterocyclic compound represented by Chemical Formula 1 is represented by any one of the followings:

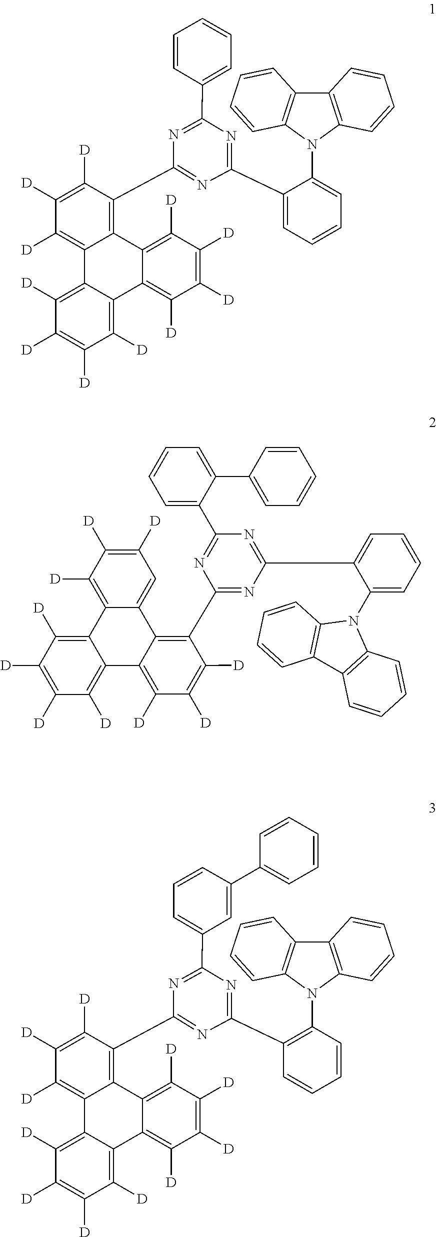 [SMILES: [2H]c1c([2H])c([2H])c2c(c1[2H])c1c([2H])c([2H])c([2H])c([2H])c1c1c(-c3nc(-c4ccccc4)nc(-c4ccccc4-n4c5ccccc5c5ccccc54)n3)c([2H])c([2H])c([2H])c21 ; [2H]c1cc2c(c([2H])c1[2H])c1c([2H])c([2H])c([2H])c([2H])c1c1c([2H])c([2H])c([2H])c(-c3nc(-c4ccccc4-c4ccccc4)nc(-c4ccccc4-n4c5ccccc5c5ccccc54)n3)c21 ; [2H]c1c([2H])c([2H])c2c(c1[2H])c1c([2H])c([2H])c([2H])c([2H])c1c1c(-c3nc(-c4cccc(-c5ccccc5)c4)nc(-c4ccccc4-n4c5ccccc5c5ccccc54)n3)c([2H])c([2H])c([2H])c21]

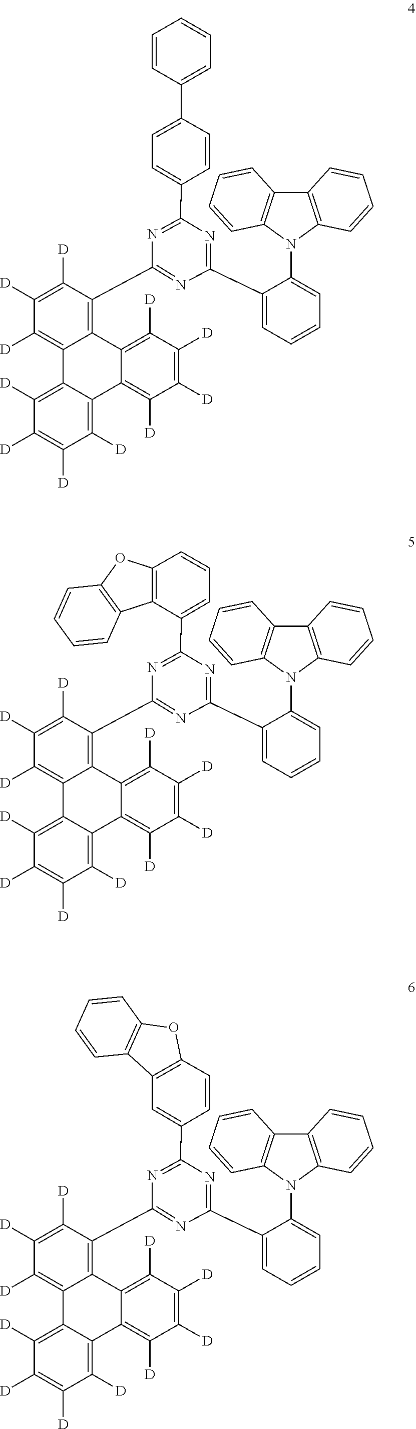 [SMILES: [2H]c1c([2H])c([2H])c2c(c1[2H])c1c([2H])c([2H])c([2H])c([2H])c1c1c(-c3nc(-c4ccc(-c5ccccc5)cc4)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5oc6ccccc6c45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc5oc6ccccc6c5c4)nc(-c4ccccc4-n4c5ccccc5c5ccccc54)n3)c([2H])c([2H])c([2H])c21]

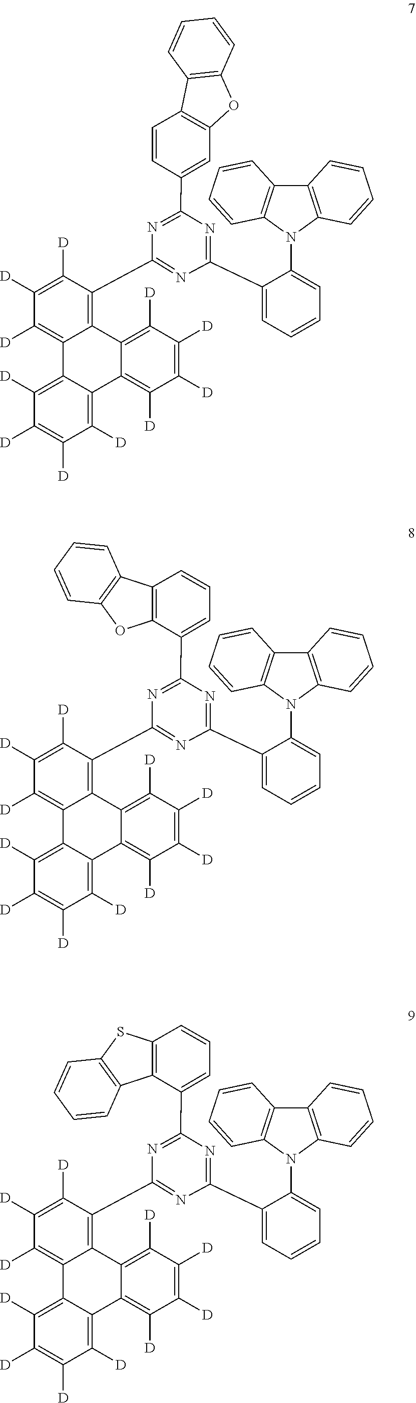 [SMILES: [2H]c1c([2H])c([2H])c2c(c1[2H])c1c([2H])c([2H])c([2H])c([2H])c1c1c(-c3nc(-c4ccc5c(c4)oc4ccccc45)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5c4oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5sc6ccccc6c45)n3)c([2H])c([2H])c([2H])c21]

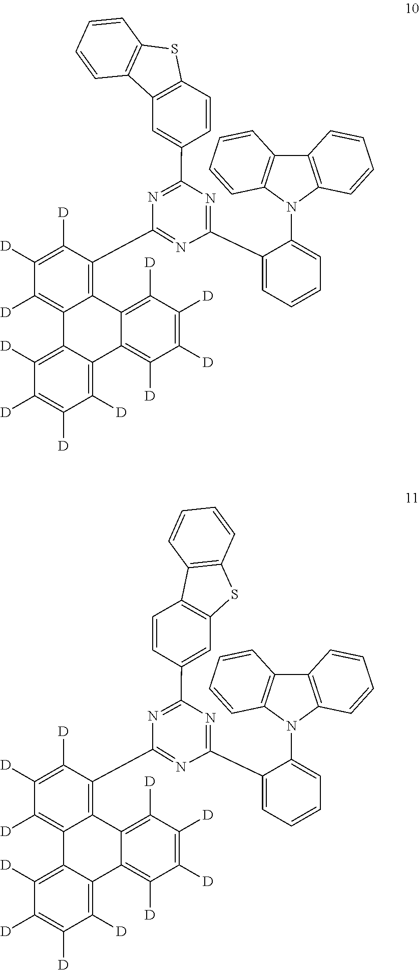 [SMILES: [2H]c1c([2H])c([2H])c2c(c1[2H])c1c([2H])c([2H])c([2H])c([2H])c1c1c(-c3nc(-c4ccc5sc6ccccc6c5c4)nc(-c4ccccc4-n4c5ccccc5c5ccccc54)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc5c(c4)sc4ccccc45)nc(-c4ccccc4-n4c5ccccc5c5ccccc54)n3)c([2H])c([2H])c([2H])c21]

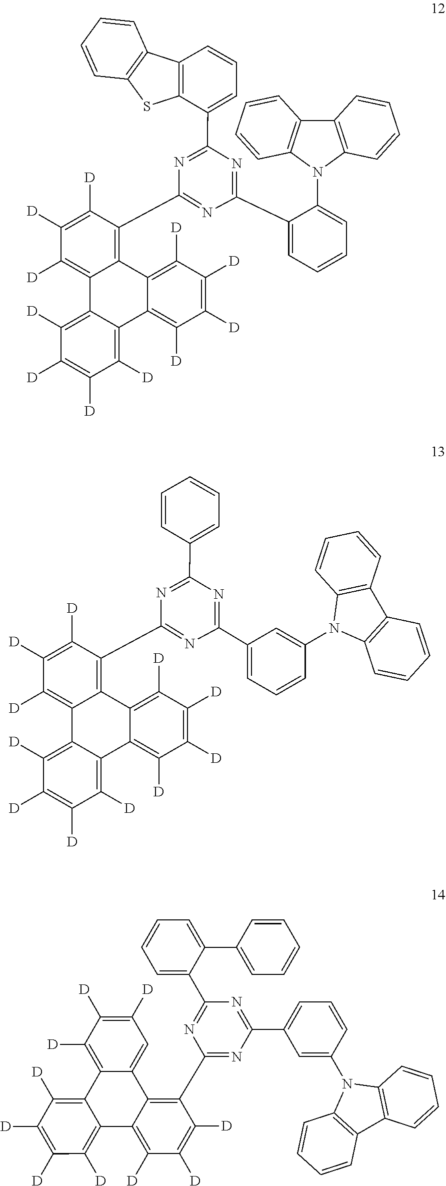 [SMILES: [2H]c1c([2H])c([2H])c2c(c1[2H])c1c([2H])c([2H])c([2H])c([2H])c1c1c(-c3nc(-c4ccccc4-n4c5ccccc5c5ccccc54)nc(-c4cccc5c4sc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4)nc(-c4cccc(-n5c6ccccc6c6ccccc65)c4)n3)c([2H])c([2H])c([2H])c21 ; [2H]c1cc2c(c([2H])c1[2H])c1c([2H])c([2H])c([2H])c([2H])c1c1c([2H])c([2H])c([2H])c(-c3nc(-c4cccc(-n5c6ccccc6c6ccccc65)c4)nc(-c4ccccc4-c4ccccc4)n3)c21]

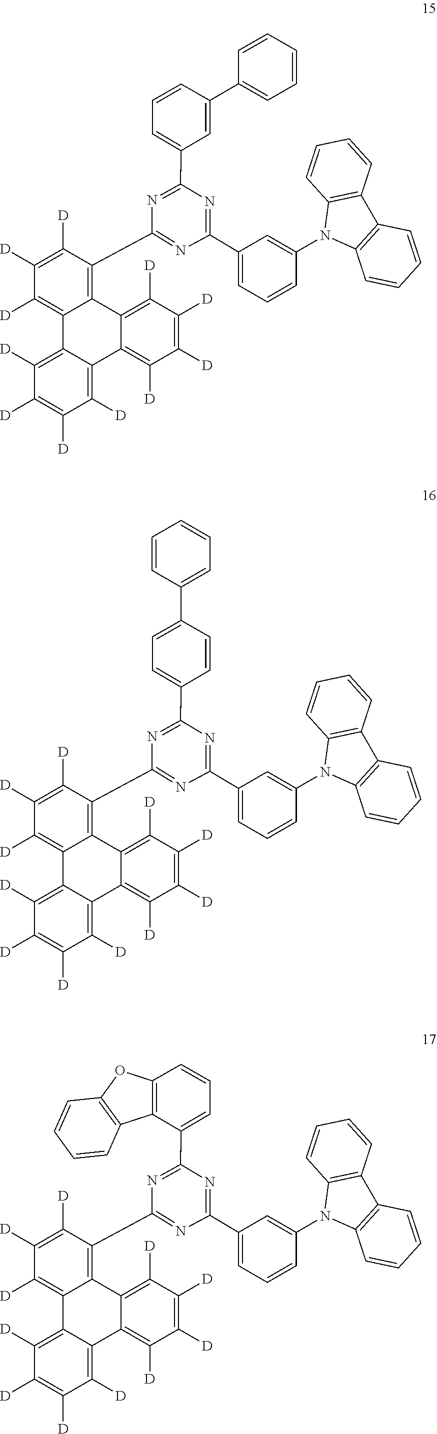 [SMILES: [2H]c1c([2H])c([2H])c2c(c1[2H])c1c([2H])c([2H])c([2H])c([2H])c1c1c(-c3nc(-c4cccc(-c5ccccc5)c4)nc(-c4cccc(-n5c6ccccc6c6ccccc65)c4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-c5ccccc5)cc4)nc(-c4cccc(-n5c6ccccc6c6ccccc65)c4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5oc6ccccc6c45)n3)c([2H])c([2H])c([2H])c21]

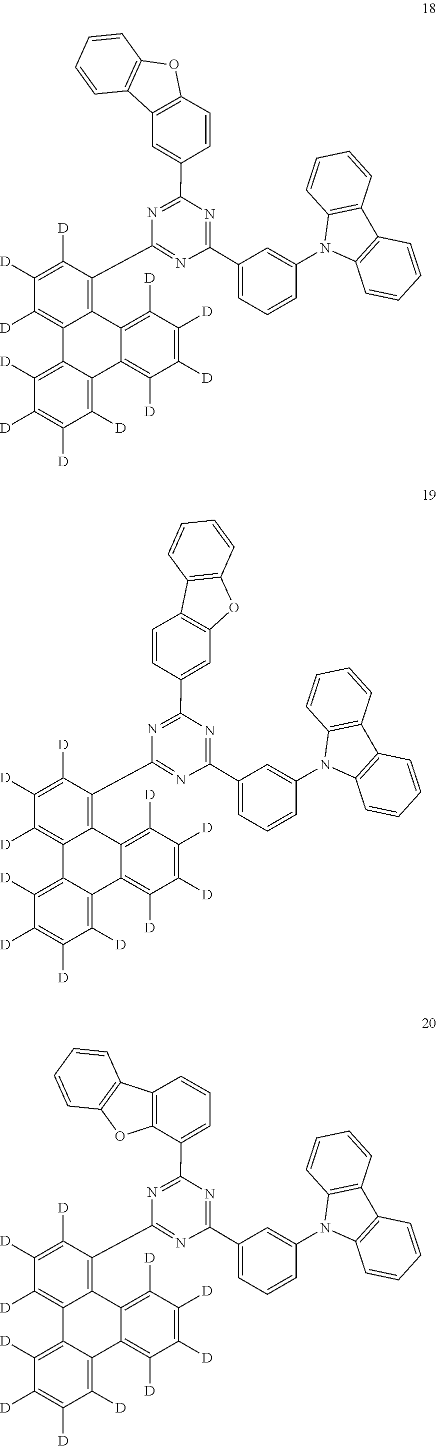 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5oc6ccccc6c5c4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5c4oc4ccccc45)n3)c([2H])c([2H])c([2H])c21]

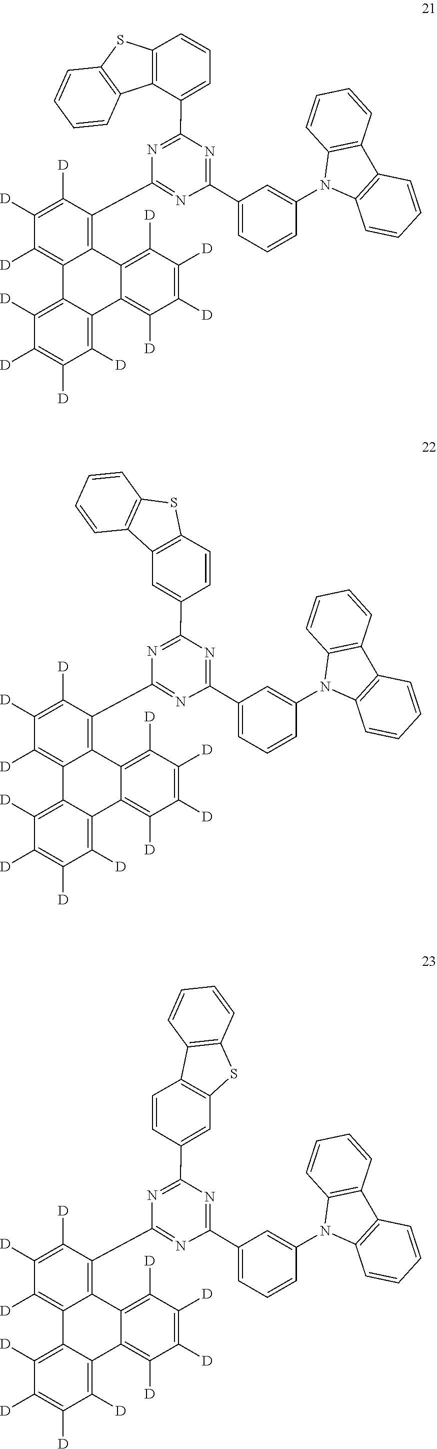 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5sc6ccccc6c45)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5sc6ccccc6c5c4)n3)c([2H])c([2H])c([2H])c21 ; [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4ccc5c(c4)sc4ccccc45)n3)c([2H])c([2H])c([2H])c21]

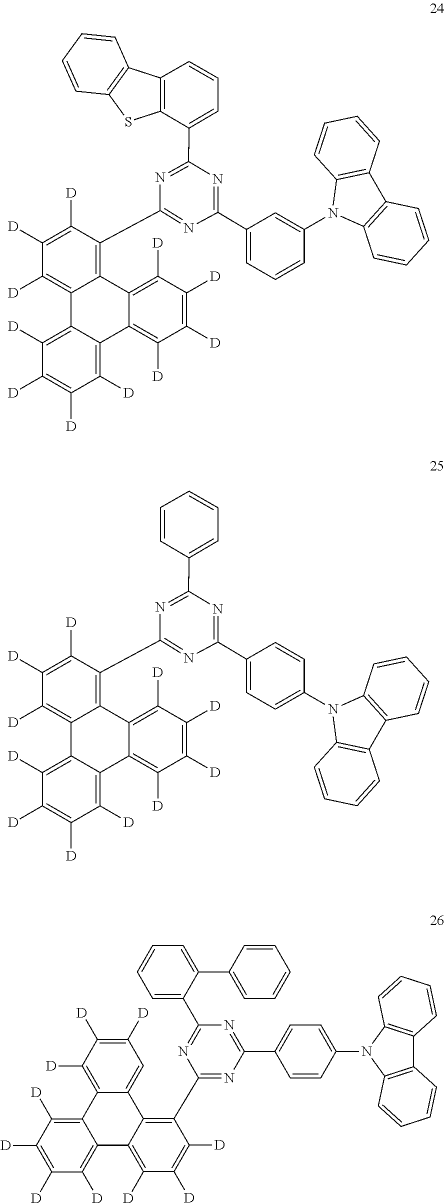 [SMILES: [2H]c1c([2H])c([2H])c2c(c1[2H])c1c([2H])c([2H])c([2H])c([2H])c1c1c(-c3nc(-c4cccc(-n5c6ccccc6c6ccccc65)c4)nc(-c4cccc5c4sc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccccc4)nc(-c4ccc(-n5c6ccccc6c6ccccc65)cc4)n3)c([2H])c([2H])c([2H])c21 ; [2H]c1cc2c(c([2H])c1[2H])c1c([2H])c([2H])c([2H])c([2H])c1c1c([2H])c([2H])c([2H])c(-c3nc(-c4ccc(-n5c6ccccc6c6ccccc65)cc4)nc(-c4ccccc4-c4ccccc4)n3)c21]

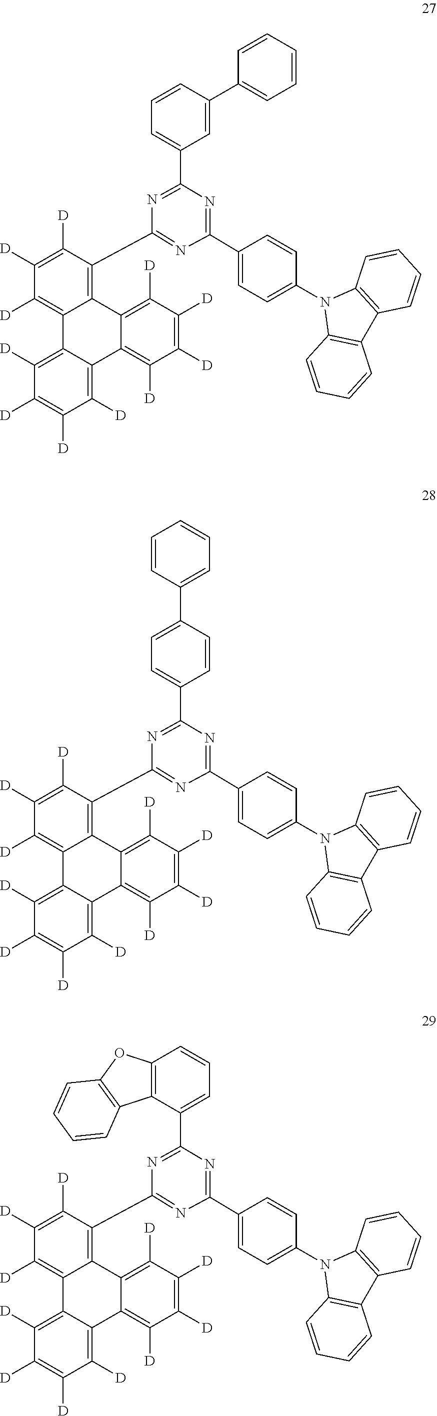 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc(-c5ccccc5)c4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-c5ccccc5)cc4)nc(-c4ccc(-n5c6ccccc6c6ccccc65)cc4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5oc6ccccc6c45)n3)c([2H])c([2H])c([2H])c21]

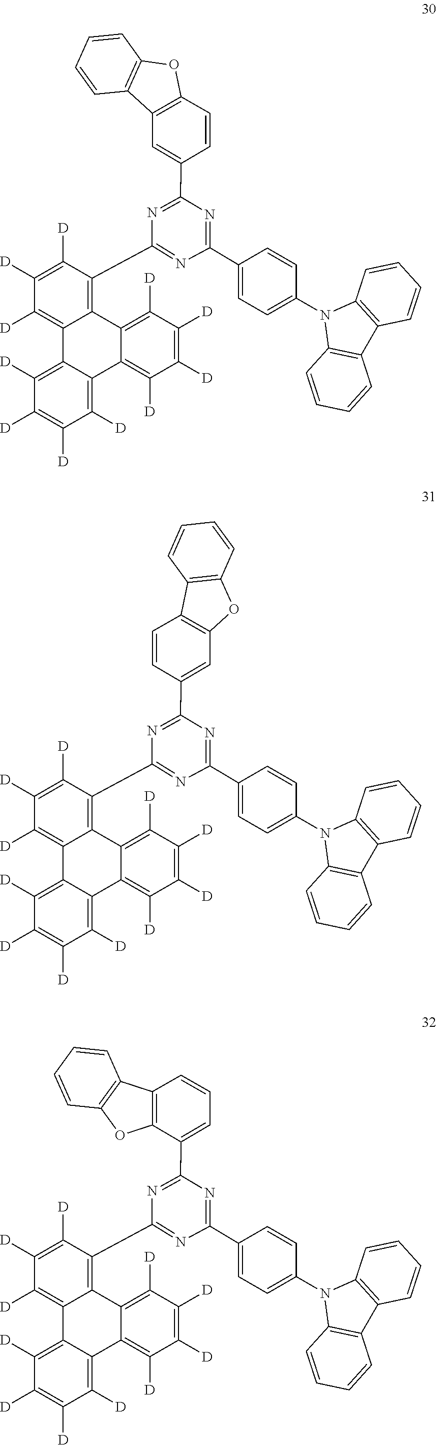 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5oc6ccccc6c5c4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5c(c4)oc4ccccc45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5c4oc4ccccc45)n3)c([2H])c([2H])c([2H])c21]

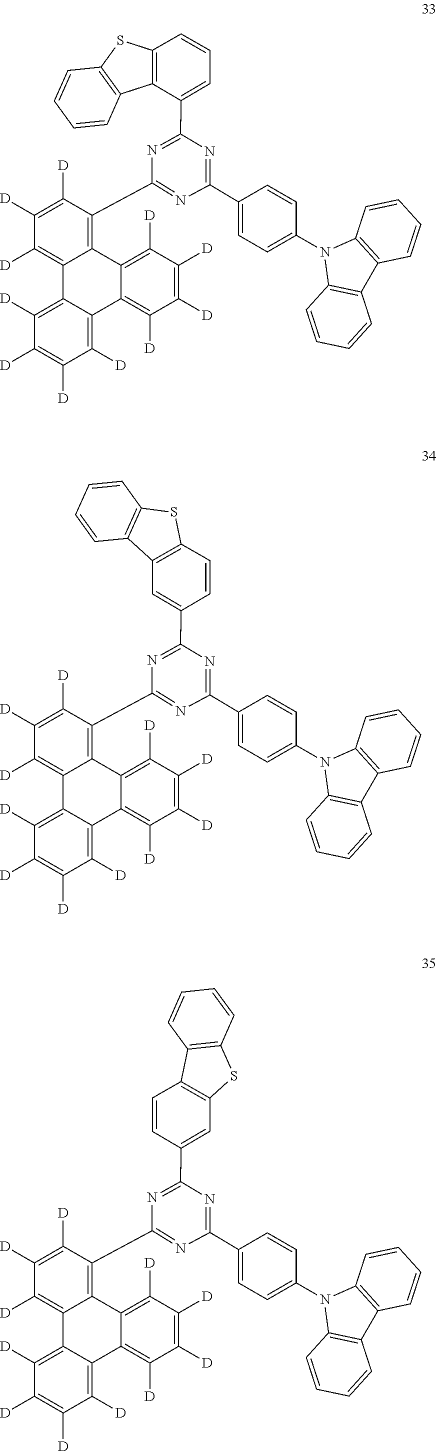 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5sc6ccccc6c45)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5sc6ccccc6c5c4)n3)c([2H])c([2H])c([2H])c21 ; [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4ccc5c(c4)sc4ccccc45)n3)c([2H])c([2H])c([2H])c21]

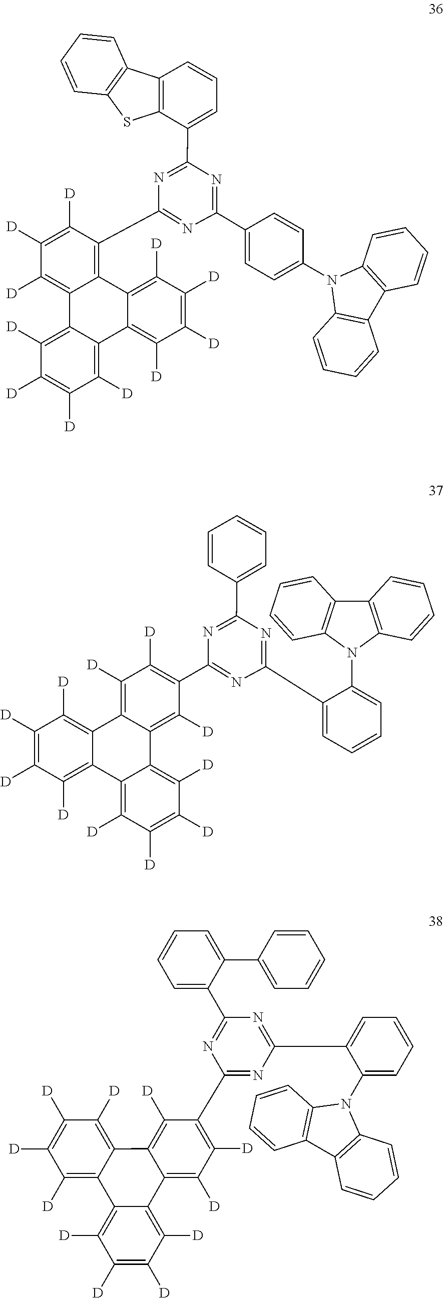 [SMILES: [2H]c1c([2H])c([2H])c2c(c1[2H])c1c([2H])c([2H])c([2H])c([2H])c1c1c(-c3nc(-c4ccc(-n5c6ccccc6c6ccccc65)cc4)nc(-c4cccc5c4sc4ccccc45)n3)c([2H])c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-n4c5ccccc5c5ccccc54)n3)c([2H])c([2H])c21]

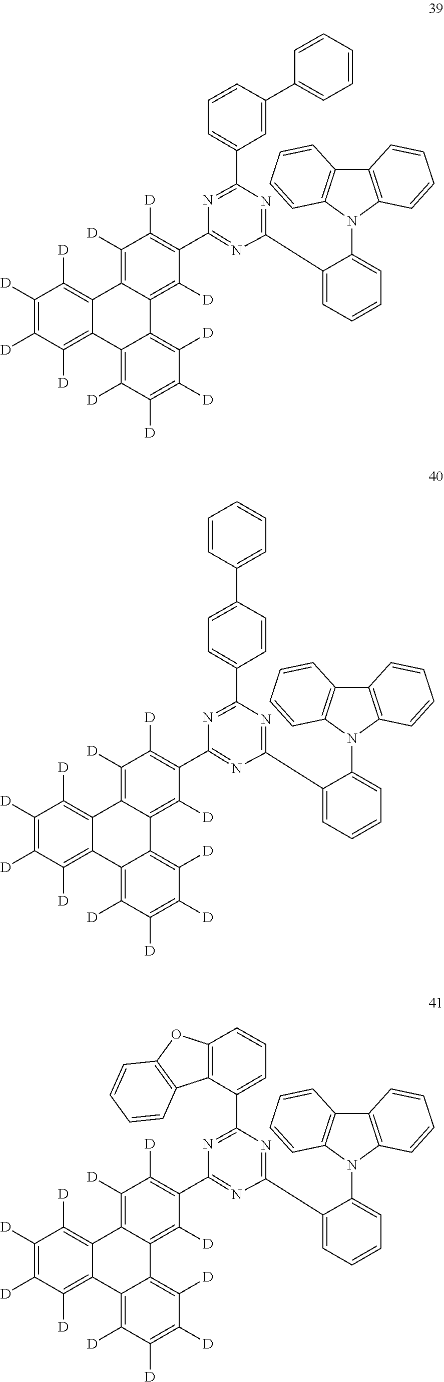 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5oc6ccccc6c45)n3)c([2H])c([2H])c21]

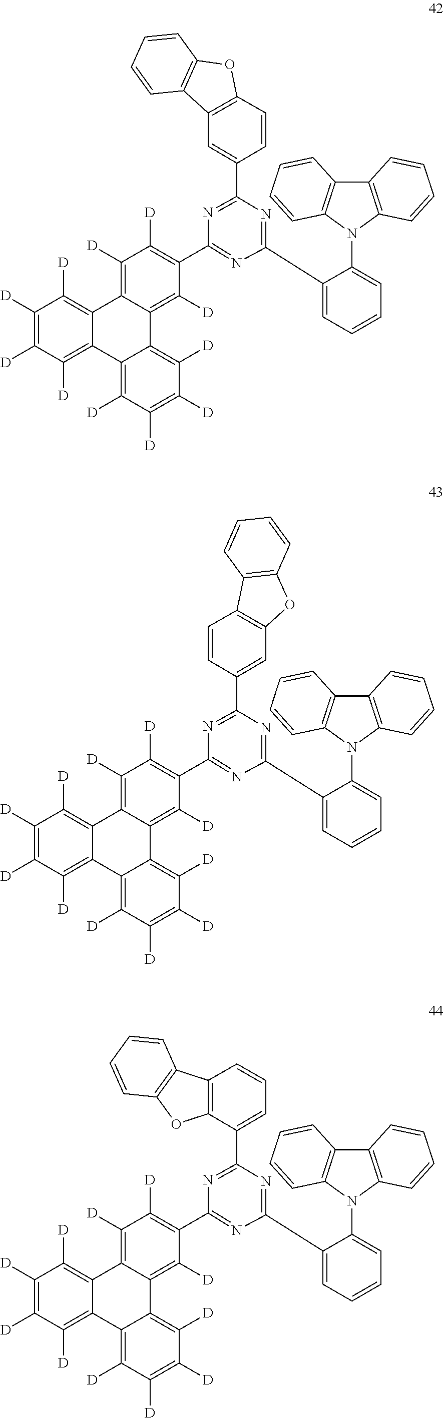 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5c4oc4ccccc45)n3)c([2H])c([2H])c21]

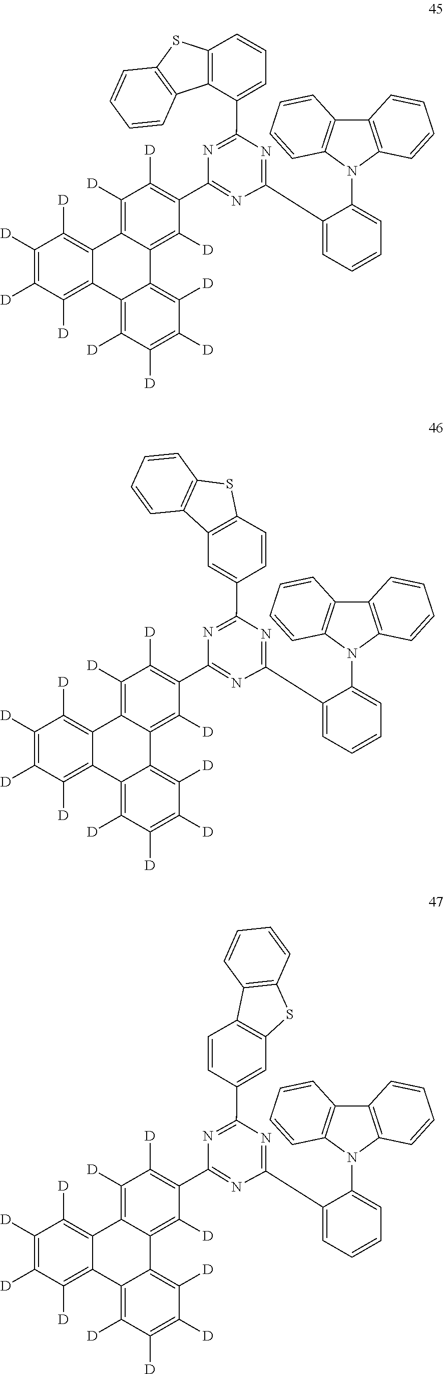 [SMILES: [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-n4c5ccccc5c5ccccc54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-n4c5ccccc5c5ccccc54)n3)c([2H])c([2H])c21]

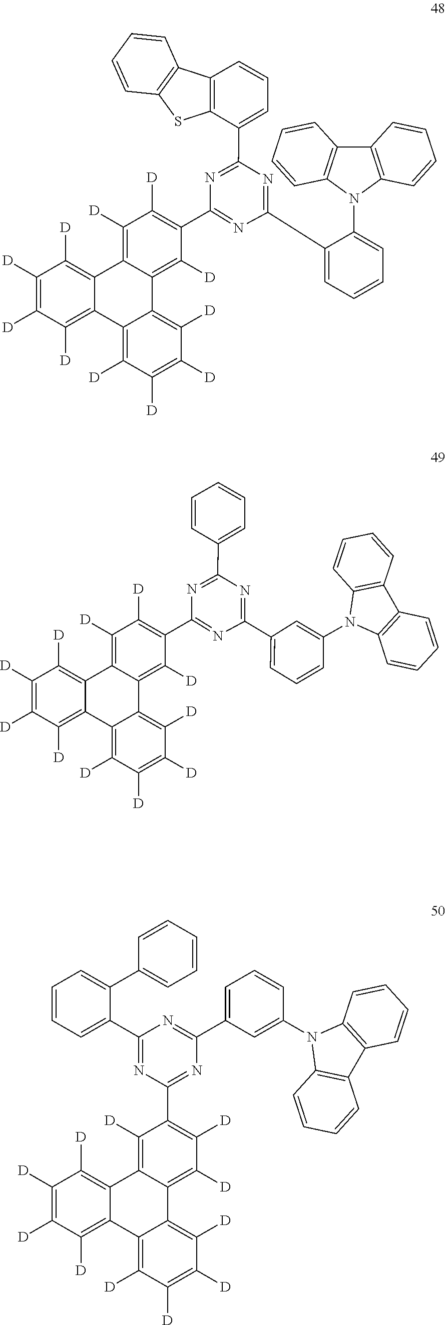 [SMILES: [2H]c1c([2H])c([2H])c2c(c1[2H])c1c([2H])c([2H])c([2H])c([2H])c1c1c([2H])c(-c3nc(-c4ccccc4-n4c5ccccc5c5ccccc54)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccccc4-c4ccccc4)n3)c([2H])c([2H])c21]

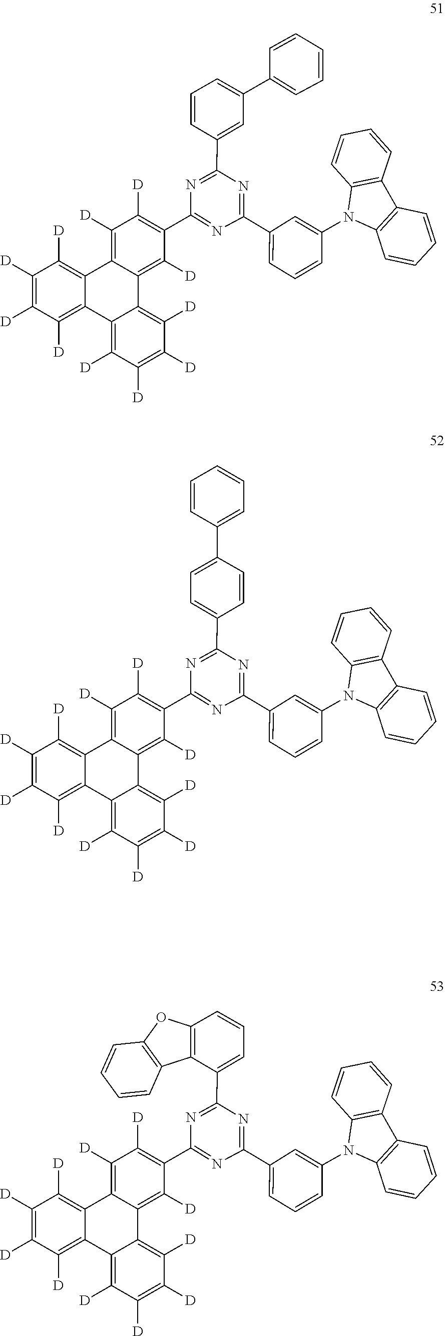 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-n5c6ccccc6c6ccccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-n5c6ccccc6c6ccccc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5oc6ccccc6c45)n3)c([2H])c([2H])c21]

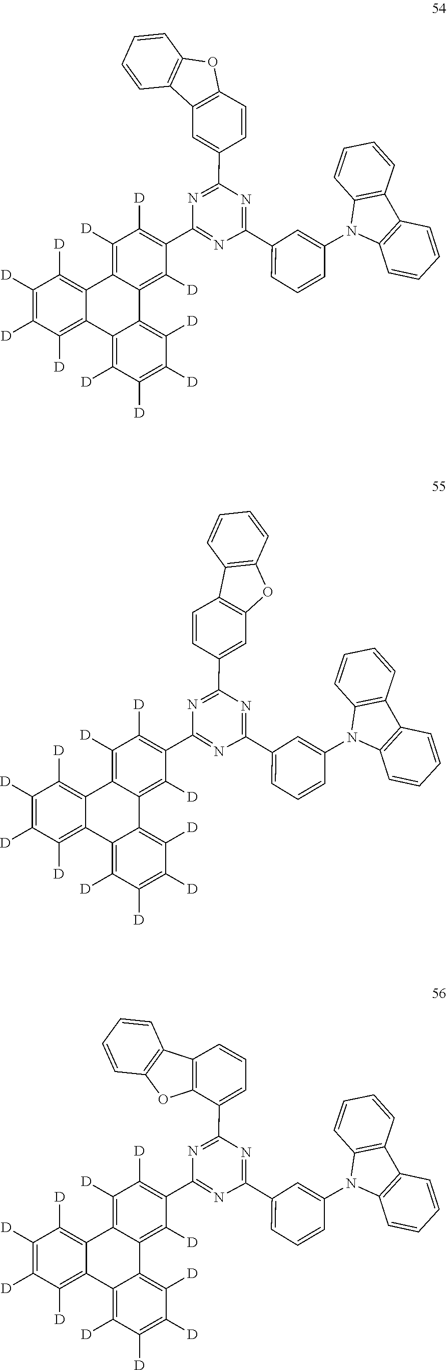 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5c4oc4ccccc45)n3)c([2H])c([2H])c21]

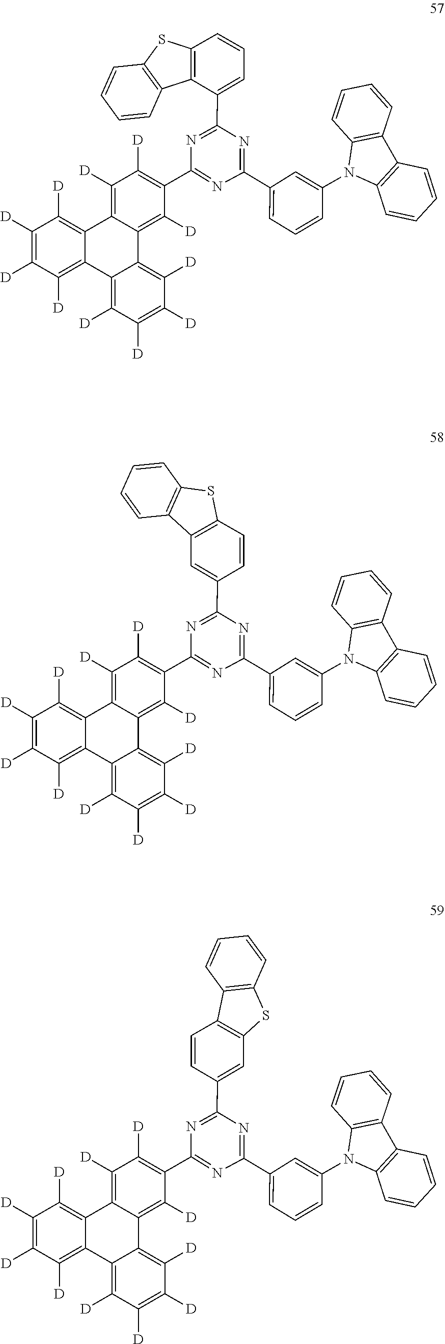 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4ccc5c(c4)sc4ccccc45)n3)c([2H])c([2H])c21]

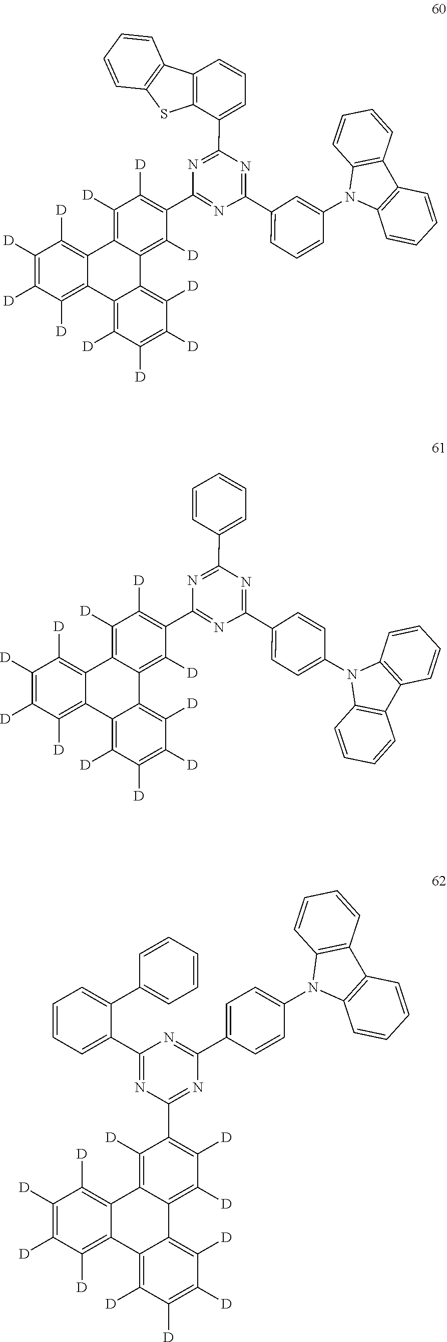 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6ccccc65)c4)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccc(-n5c6ccccc6c6ccccc65)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccccc4-c4ccccc4)n3)c([2H])c([2H])c21]

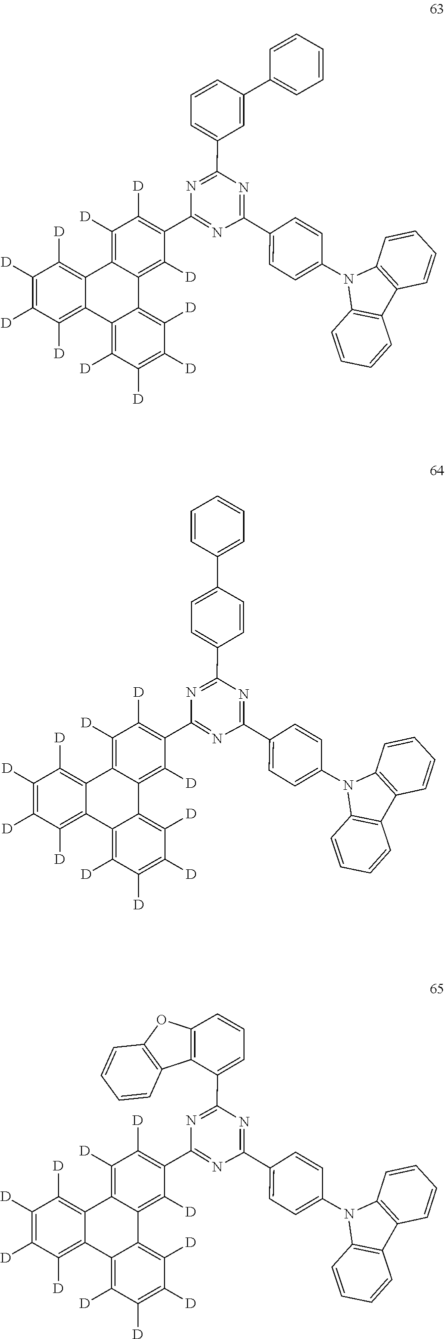 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-n5c6ccccc6c6ccccc65)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5oc6ccccc6c45)n3)c([2H])c([2H])c21]

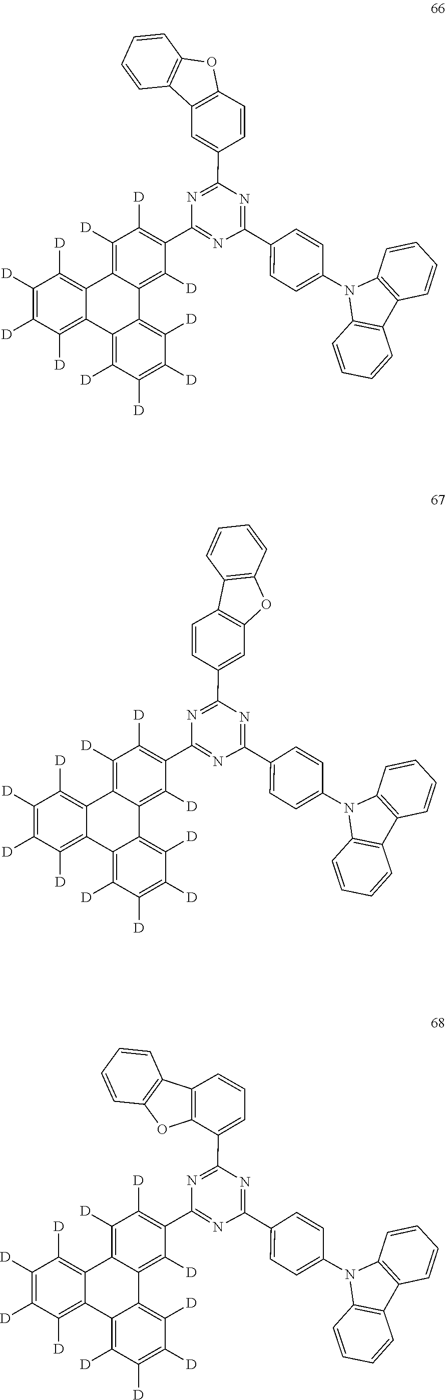 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5c4oc4ccccc45)n3)c([2H])c([2H])c21]

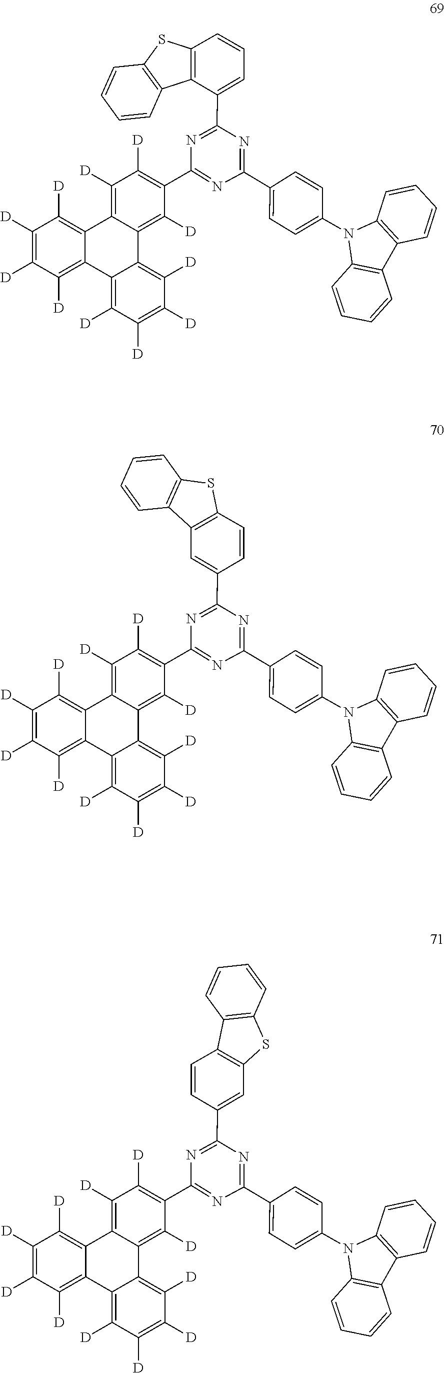 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4ccc5c(c4)sc4ccccc45)n3)c([2H])c([2H])c21]

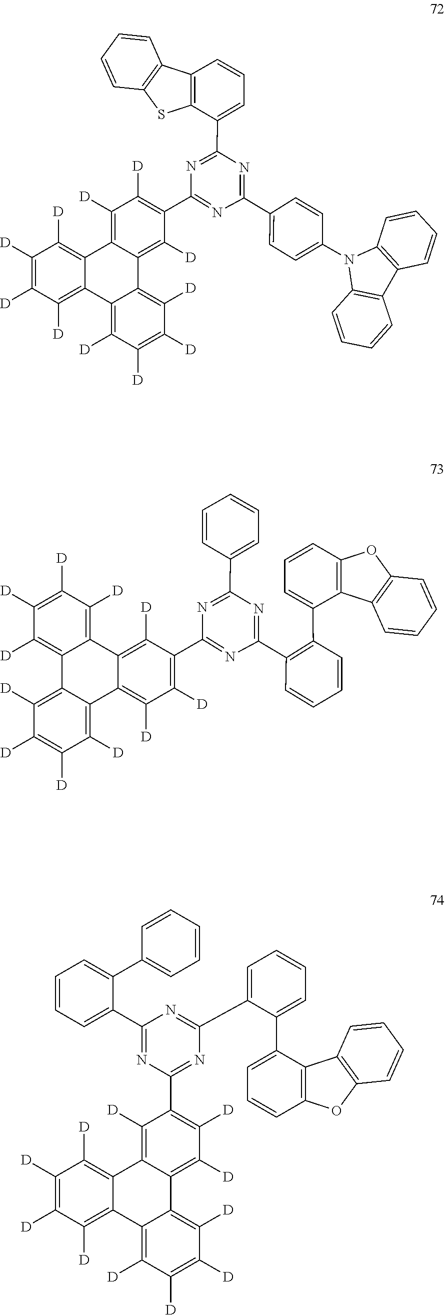 [SMILES: [2H]c1c([2H])c([2H])c2c(c1[2H])c1c([2H])c([2H])c([2H])c([2H])c1c1c([2H])c(-c3nc(-c4ccc(-n5c6ccccc6c6ccccc65)cc4)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5oc6ccccc6c45)n3)c([2H])c([2H])c21]

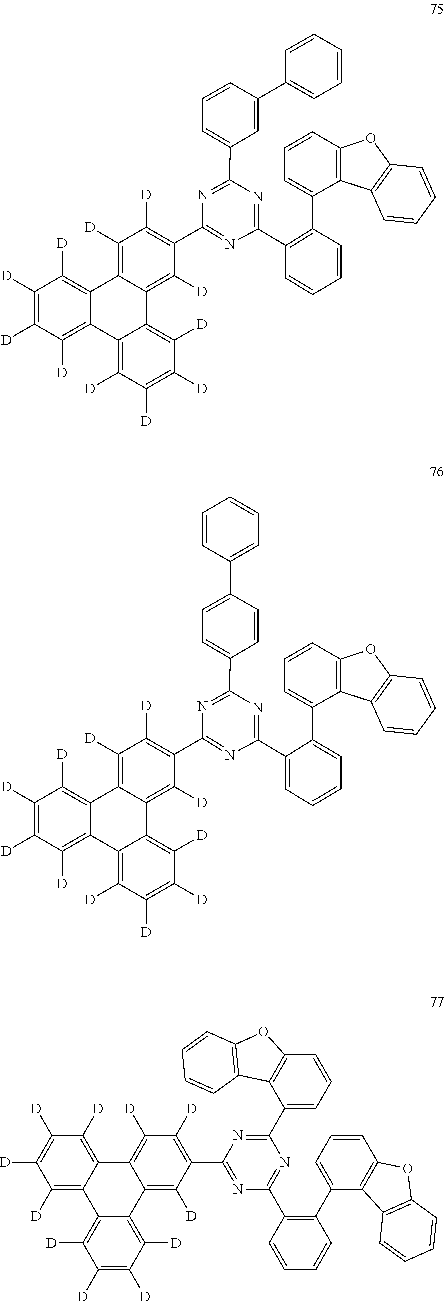 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5oc6ccccc6c45)n3)c([2H])c([2H])c21]

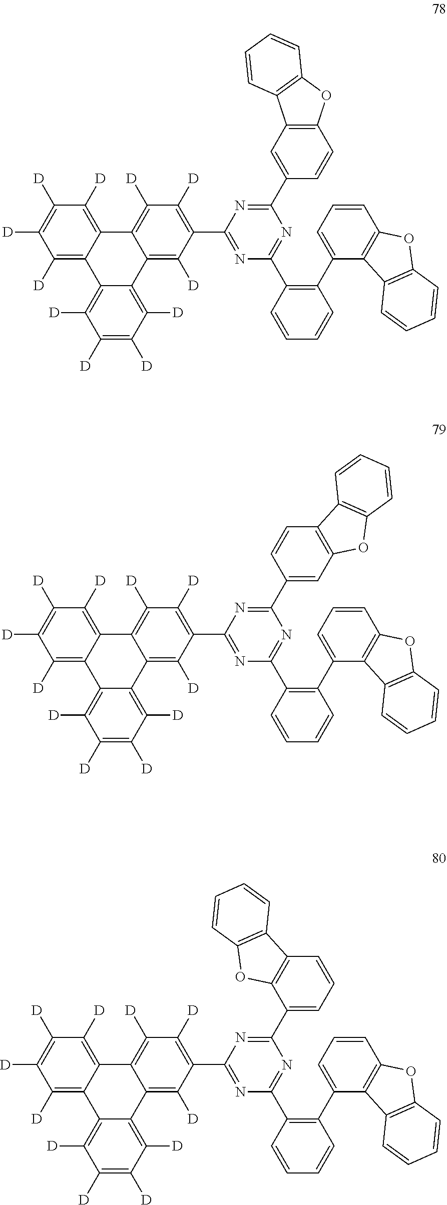 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5c4oc4ccccc45)n3)c([2H])c([2H])c21]

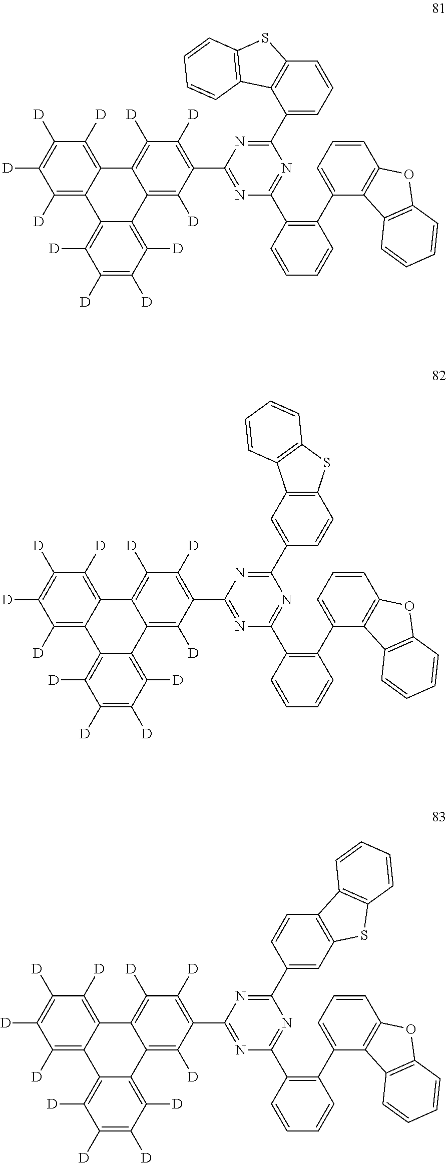 [SMILES: [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5oc6ccccc6c45)n3)c([2H])c([2H])c21]

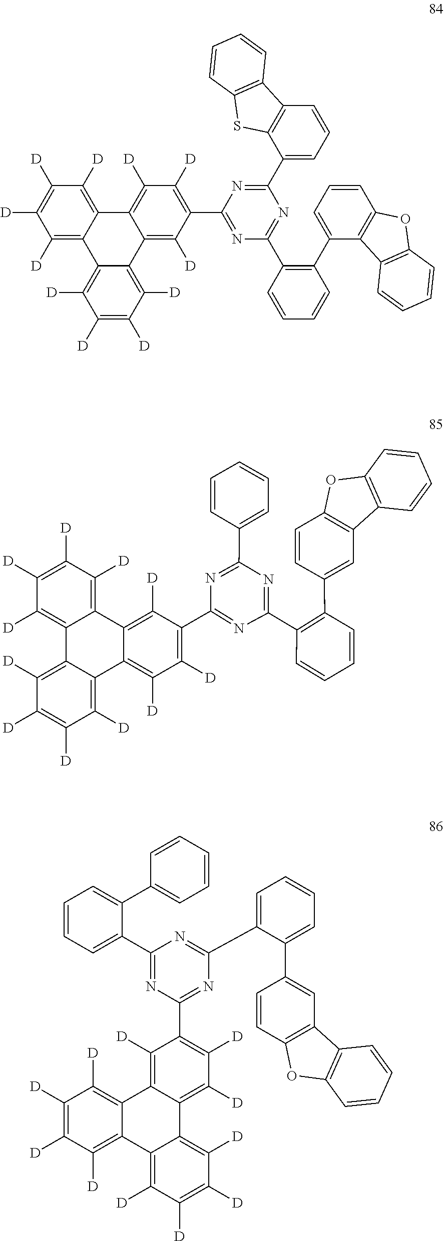 [SMILES: [2H]c1c([2H])c([2H])c2c(c1[2H])c1c([2H])c([2H])c([2H])c([2H])c1c1c([2H])c(-c3nc(-c4ccccc4-c4cccc5oc6ccccc6c45)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5oc6ccccc6c5c4)n3)c([2H])c([2H])c21]

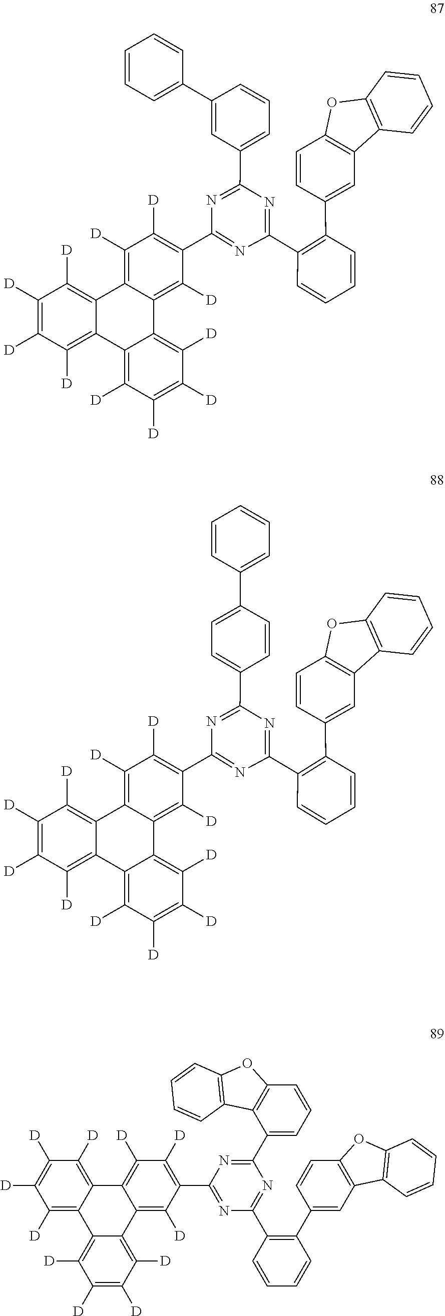 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5oc6ccccc6c5c4)nc(-c4cccc5oc6ccccc6c45)n3)c([2H])c([2H])c21]

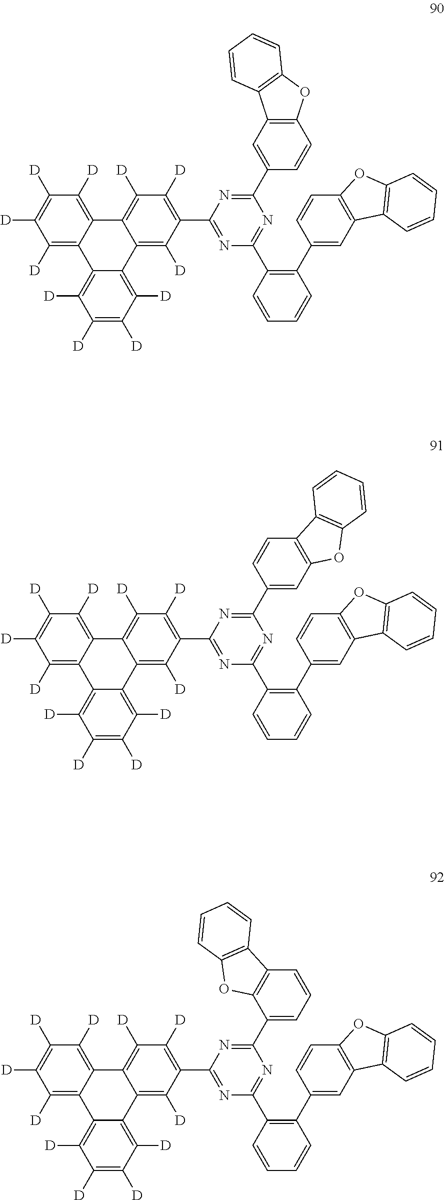 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5oc6ccccc6c5c4)nc(-c4cccc5c4oc4ccccc45)n3)c([2H])c([2H])c21]

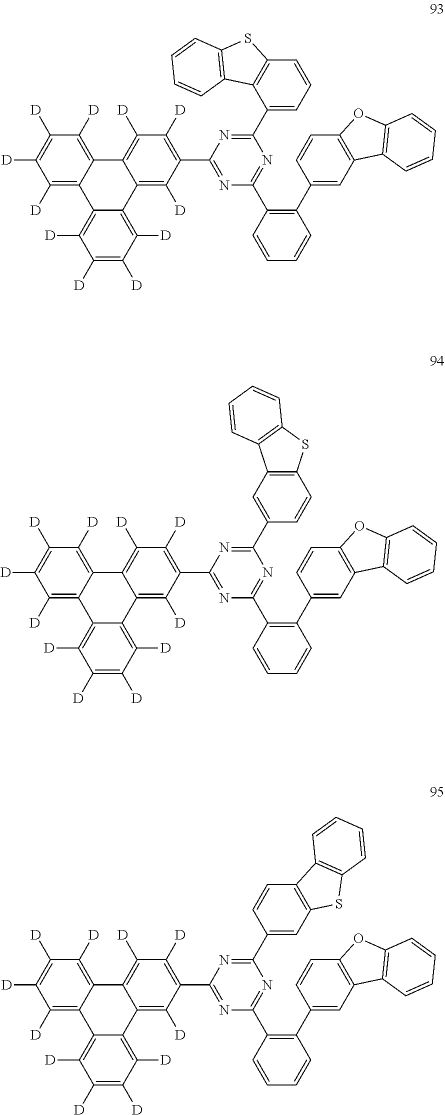 [SMILES: [2H]c1c([2H])c([2H])c2c(c1[2H])c1c([2H])c([2H])c([2H])c([2H])c1c1c([2H])c(-c3nc(-c4ccccc4-c4ccc5oc6ccccc6c5c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4ccc5oc6ccccc6c5c4)n3)c([2H])c([2H])c21]

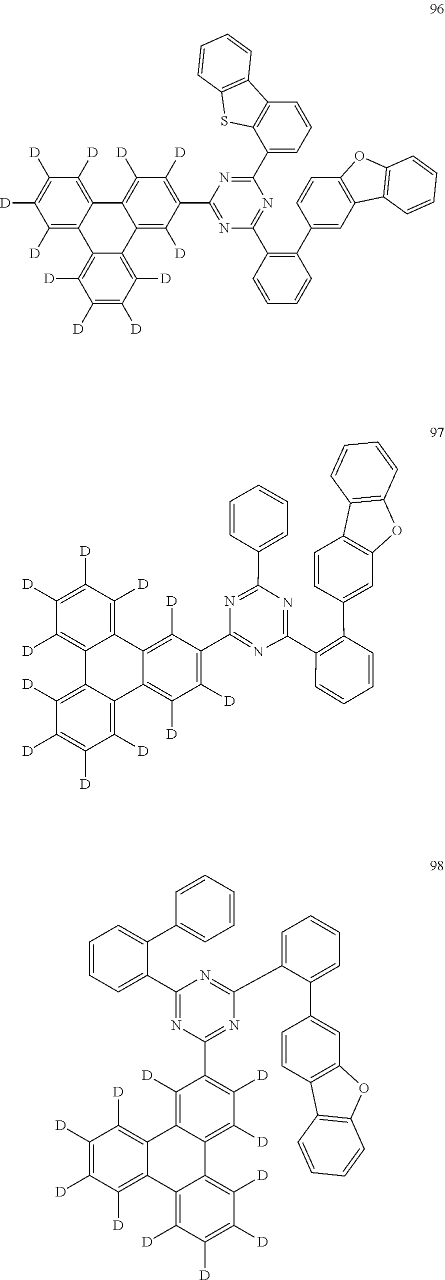 [SMILES: [2H]c1c([2H])c([2H])c2c(c1[2H])c1c([2H])c([2H])c([2H])c([2H])c1c1c([2H])c(-c3nc(-c4ccccc4-c4ccc5oc6ccccc6c5c4)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c(c4)oc4ccccc45)n3)c([2H])c([2H])c21]

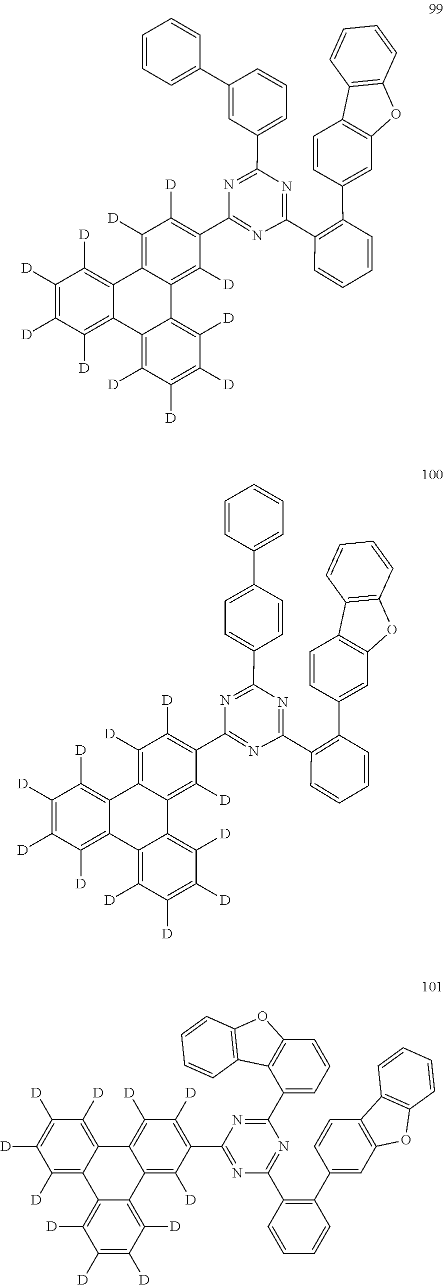 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5oc6ccccc6c45)n3)c([2H])c([2H])c21]

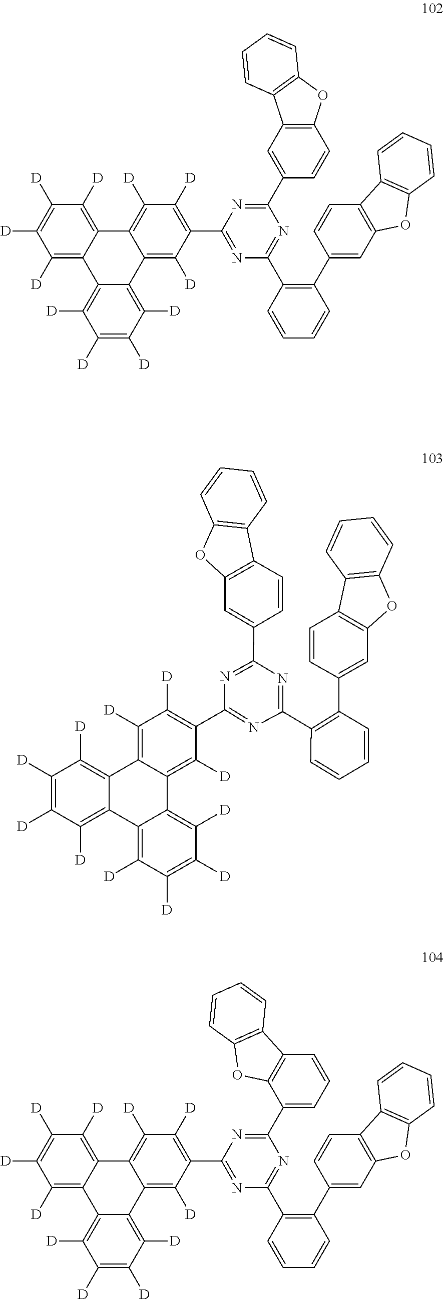 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5c4oc4ccccc45)n3)c([2H])c([2H])c21]

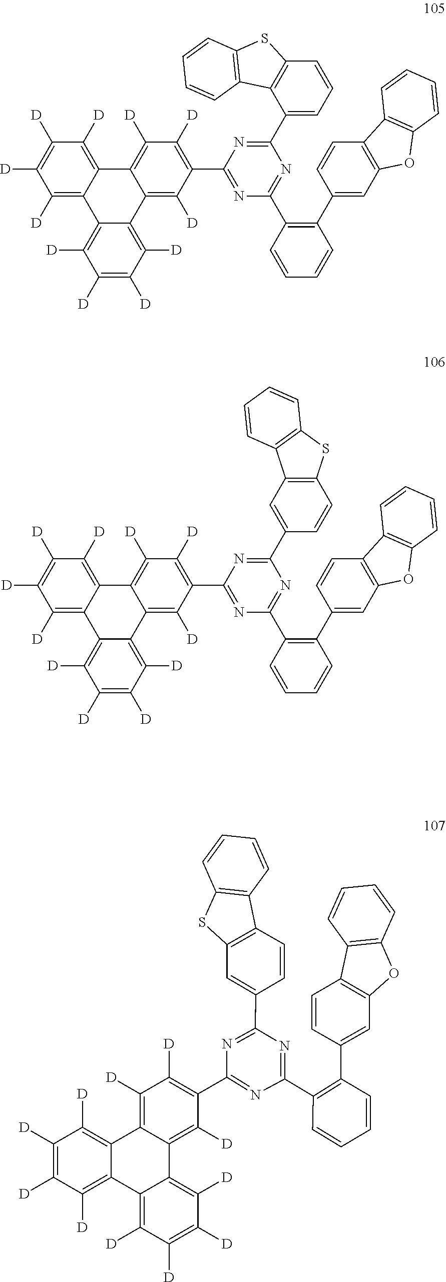 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4ccc5c(c4)oc4ccccc45)n3)c([2H])c([2H])c21]

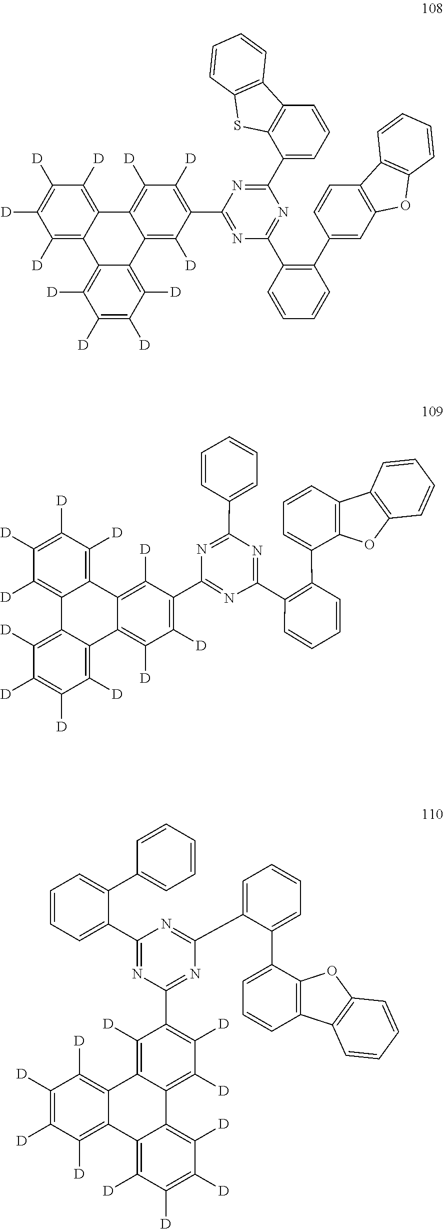 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)oc4ccccc45)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c4oc4ccccc45)n3)c([2H])c([2H])c21]

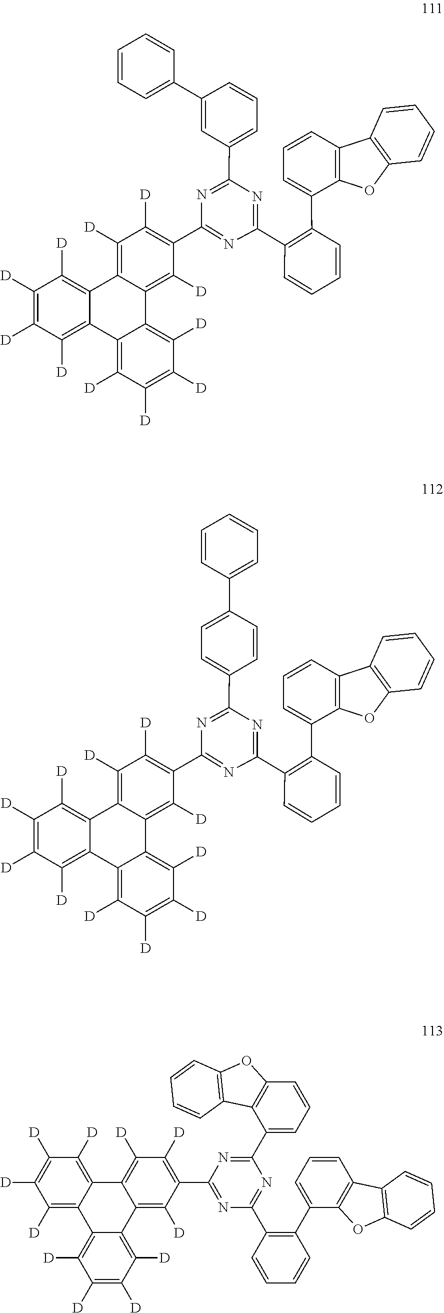 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5oc6ccccc6c45)n3)c([2H])c([2H])c21]

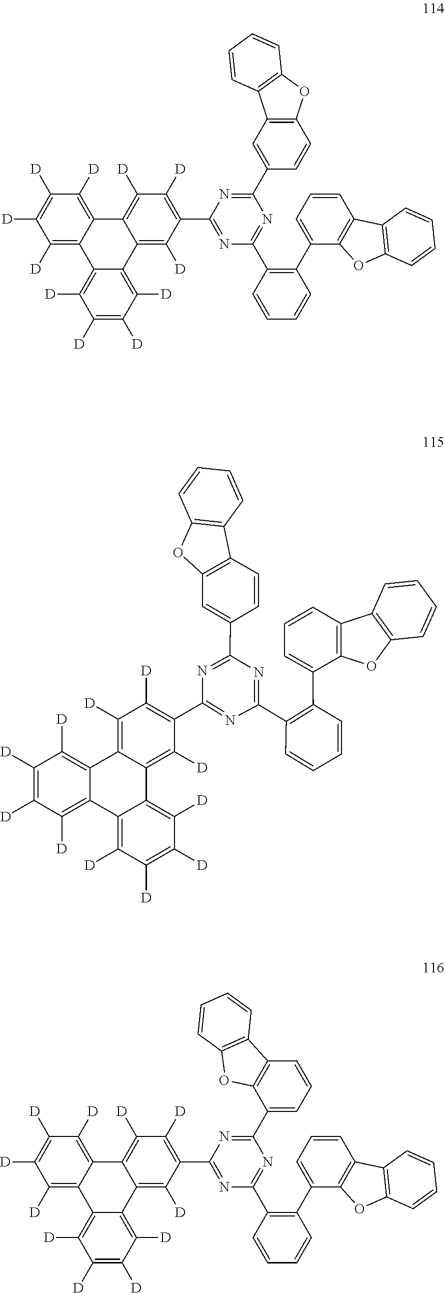 [SMILES: [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5c4oc4ccccc45)n3)c([2H])c([2H])c21]

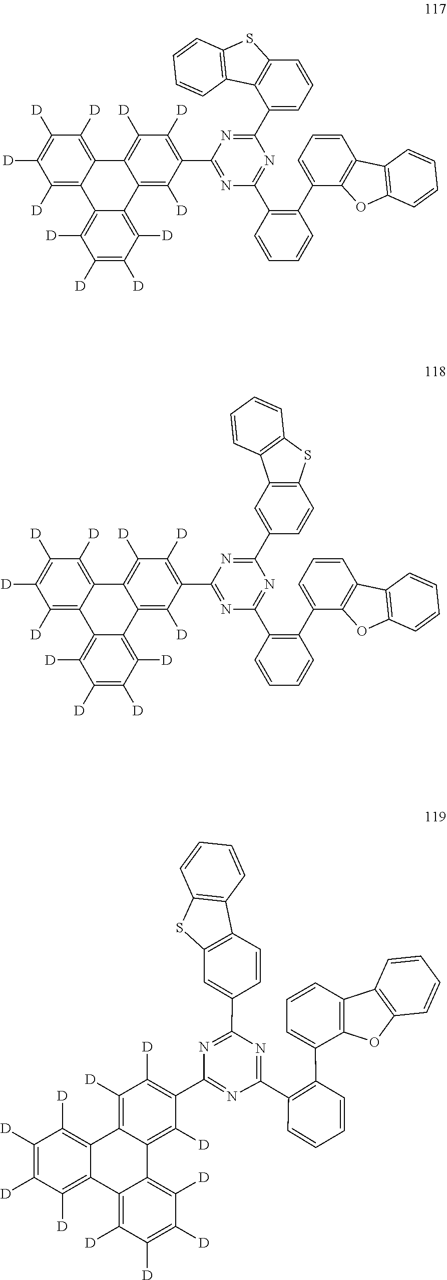 [SMILES: [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5c4oc4ccccc45)n3)c([2H])c([2H])c21]

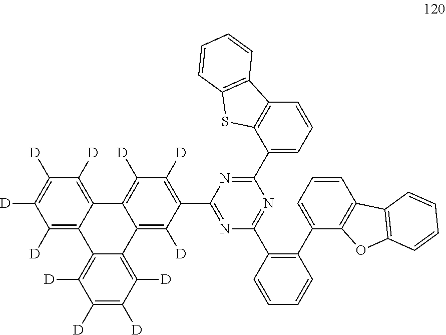 [SMILES: [2H]c1c([2H])c([2H])c2c(c1[2H])c1c([2H])c([2H])c([2H])c([2H])c1c1c([2H])c(-c3nc(-c4ccccc4-c4cccc5c4oc4ccccc45)nc(-c4cccc5c4sc4ccccc45)n3)c([2H])c([2H])c21]

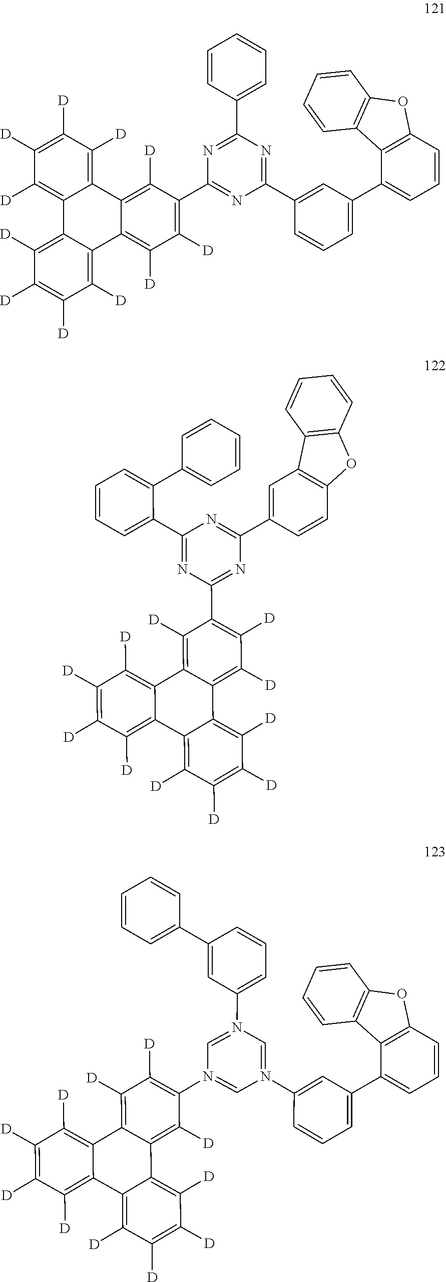

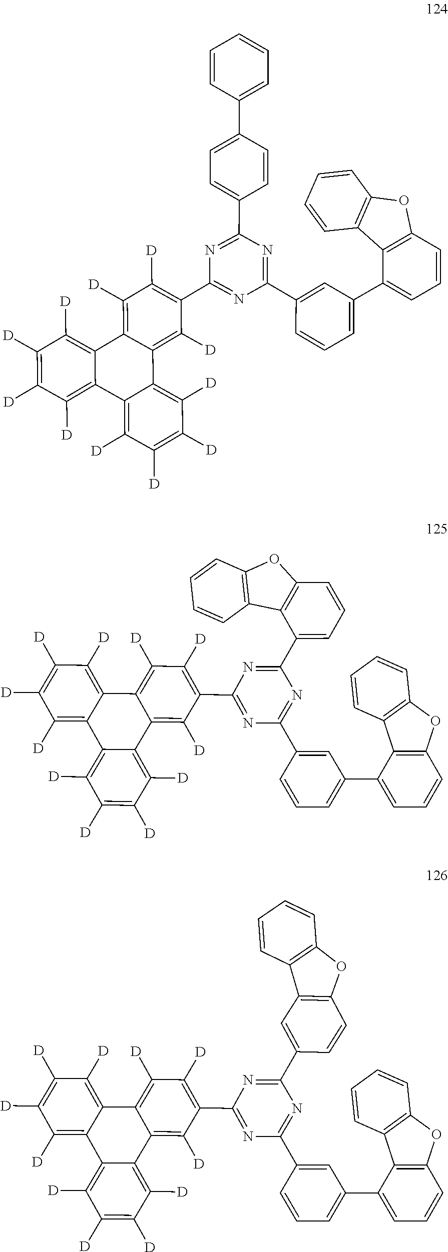 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5cccc6oc7ccccc7c56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4ccc5oc6ccccc6c5c4)n3)c([2H])c([2H])c21]

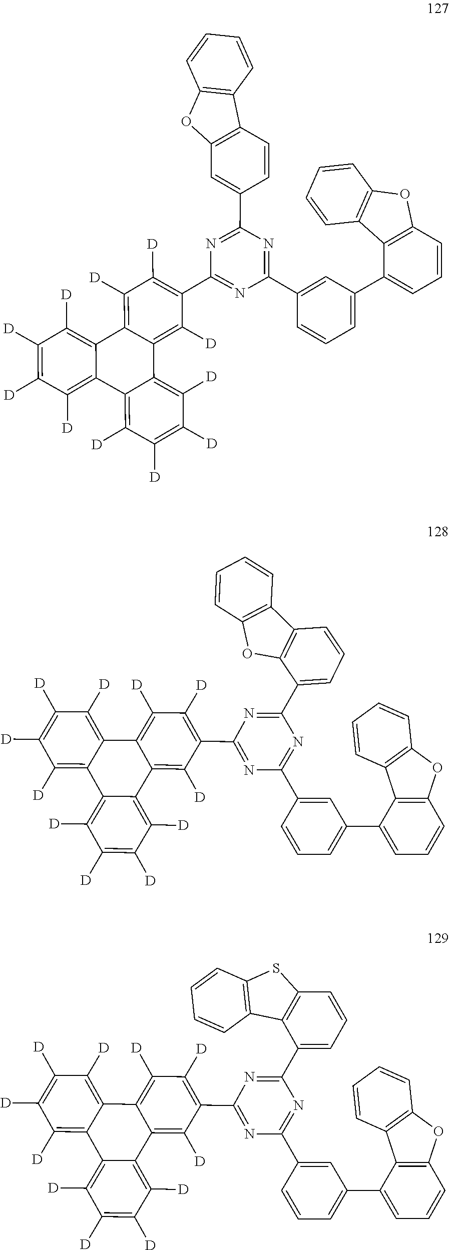 [SMILES: [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4cccc5sc6ccccc6c45)n3)c([2H])c([2H])c21]

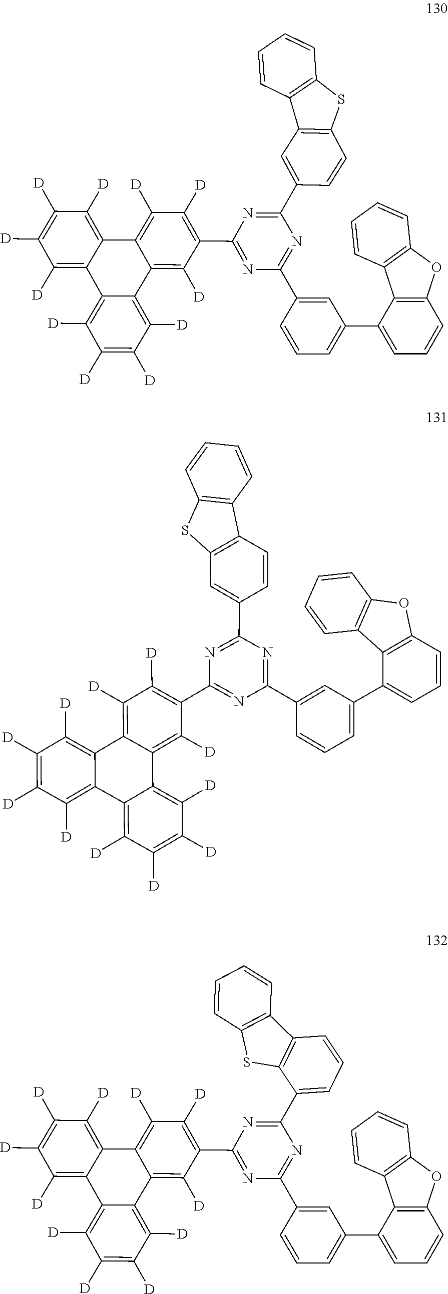 [SMILES: [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6oc7ccccc7c56)c4)nc(-c4cccc5c4sc4ccccc45)n3)c([2H])c([2H])c21]

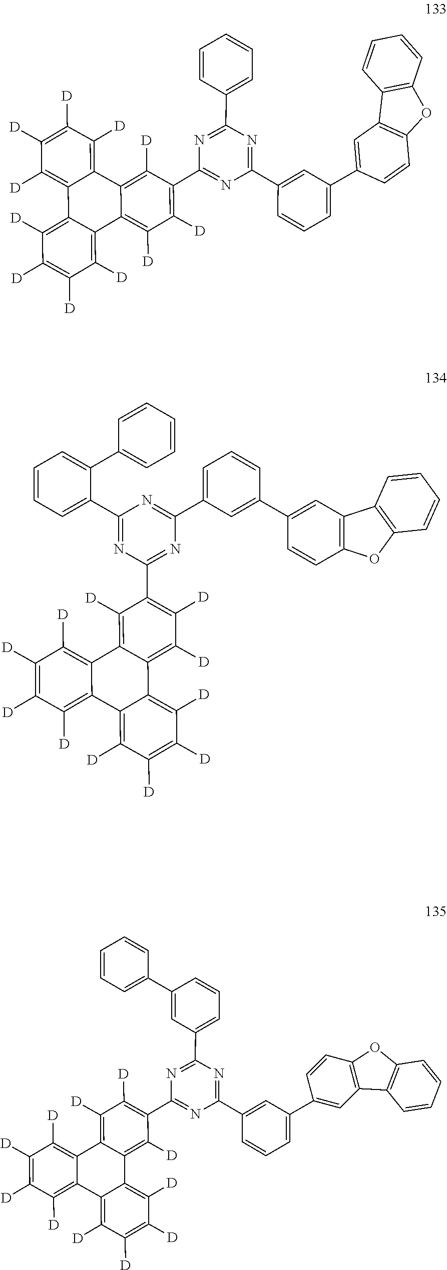 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6oc7ccccc7c6c5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6oc7ccccc7c6c5)c4)n3)c([2H])c([2H])c21]

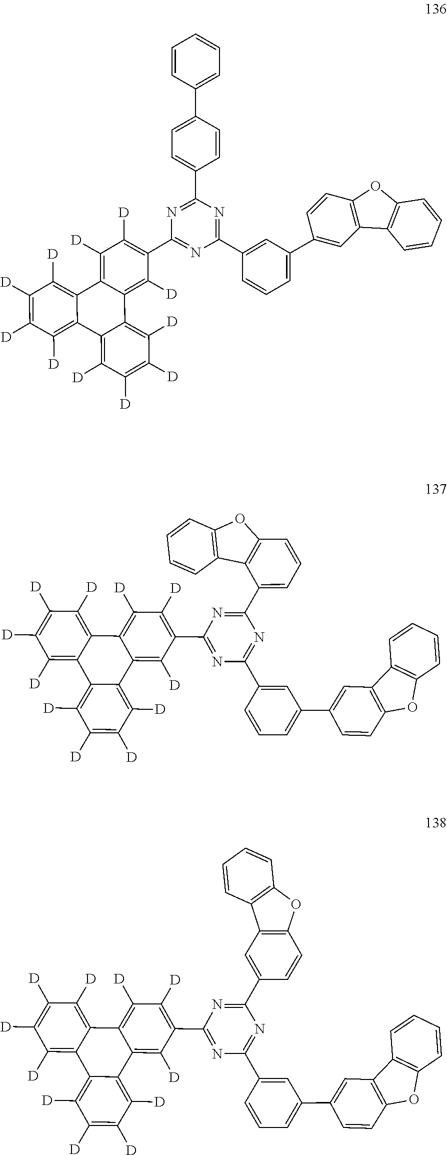 [SMILES: [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6oc7ccccc7c6c5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4ccc5oc6ccccc6c5c4)n3)c([2H])c([2H])c21]

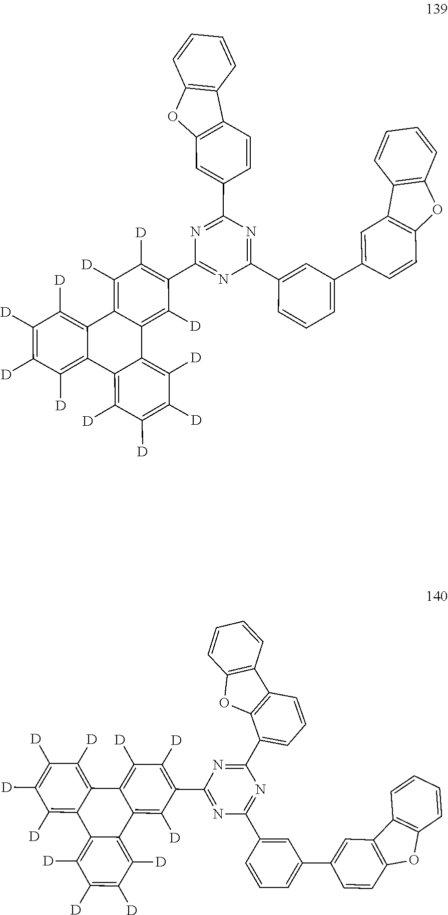 [SMILES: [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4cccc5c4oc4ccccc45)n3)c([2H])c([2H])c21]

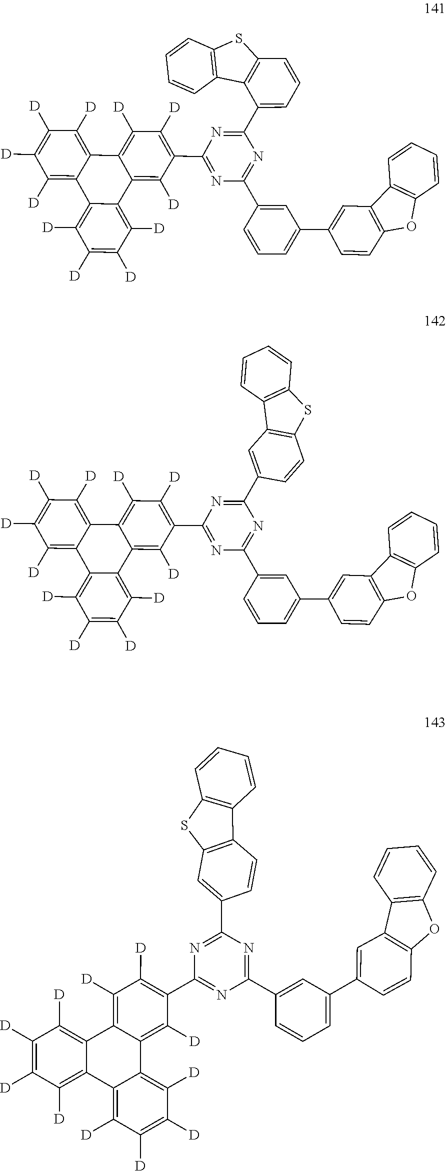 [SMILES: [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4ccc5sc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4ccc5c(c4)sc4ccccc45)n3)c([2H])c([2H])c21]

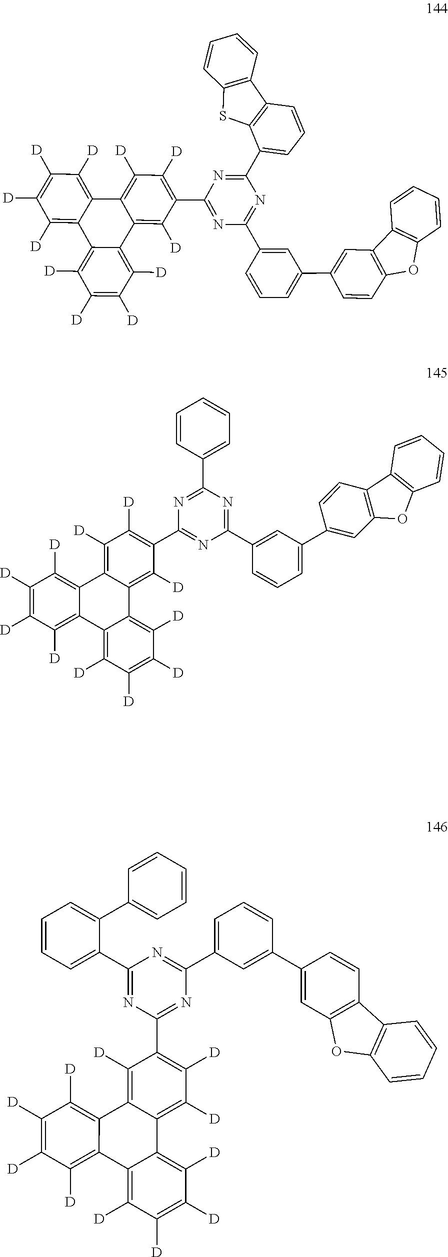 [SMILES: [2H]c1c([2H])c([2H])c2c(c1[2H])c1c([2H])c([2H])c([2H])c([2H])c1c1c([2H])c(-c3nc(-c4cccc(-c5ccc6oc7ccccc7c6c5)c4)nc(-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6c(c5)oc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4ccccc4-c4ccccc4)n3)c([2H])c([2H])c21]

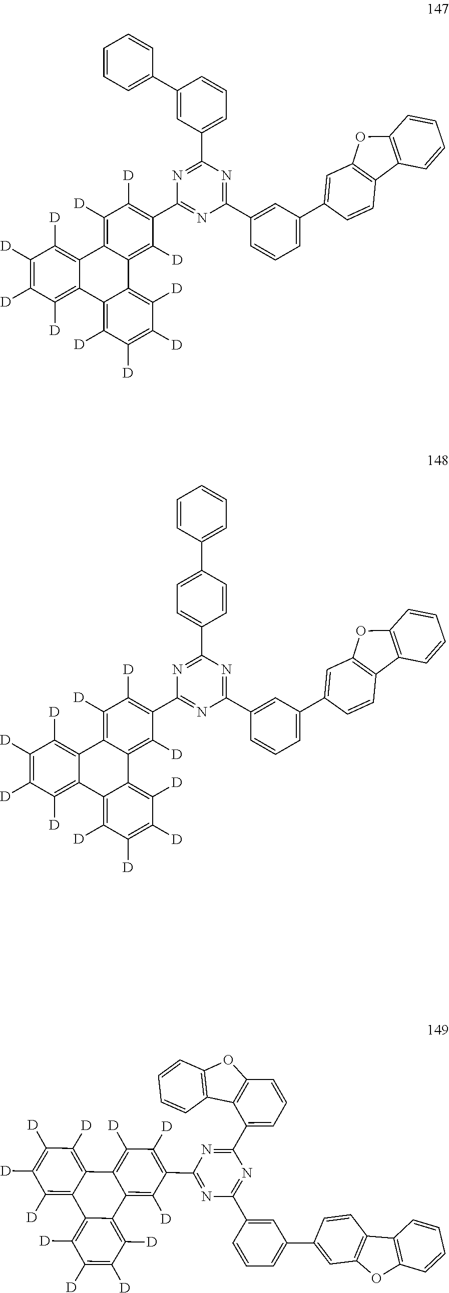 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6c(c5)oc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6c(c5)oc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4cccc5oc6ccccc6c45)n3)c([2H])c([2H])c21]

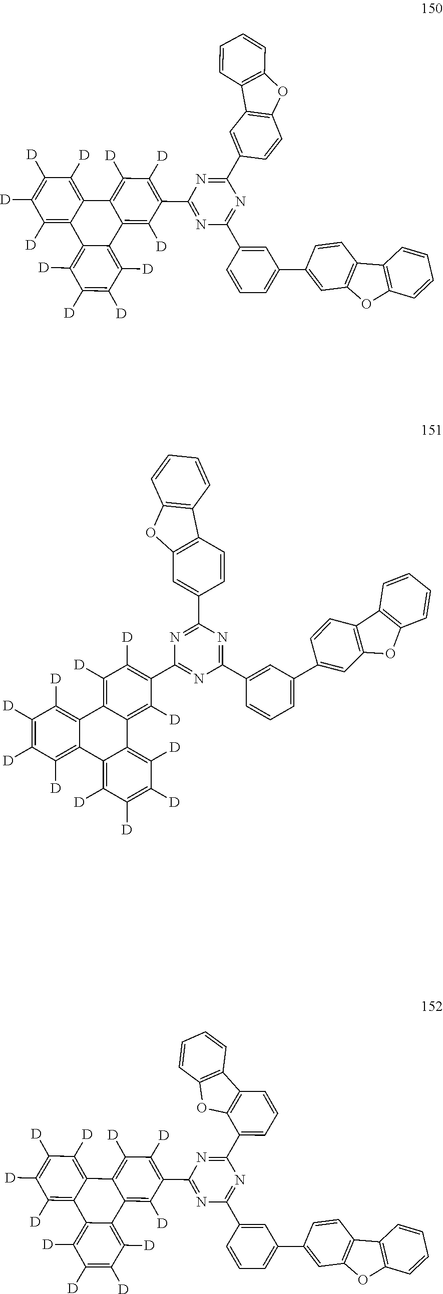 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4ccc5oc6ccccc6c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)oc5ccccc56)c4)nc(-c4cccc5c4oc4ccccc45)n3)c([2H])c([2H])c21]

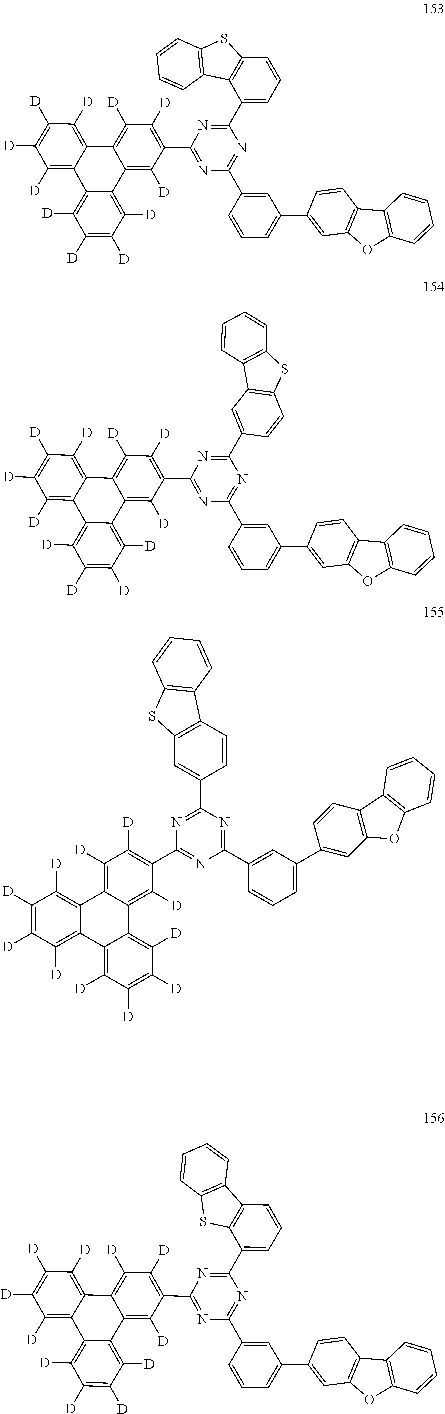

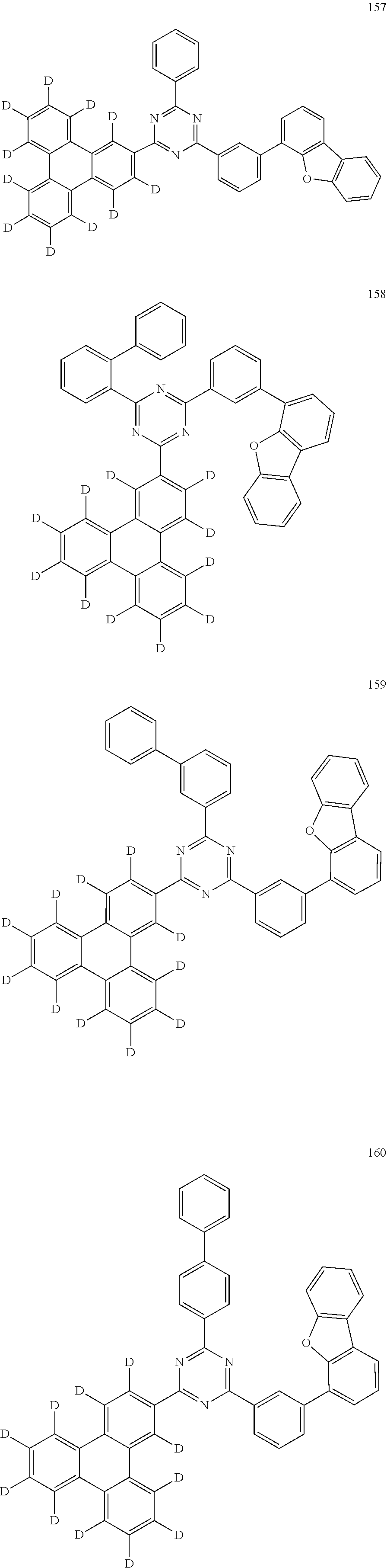

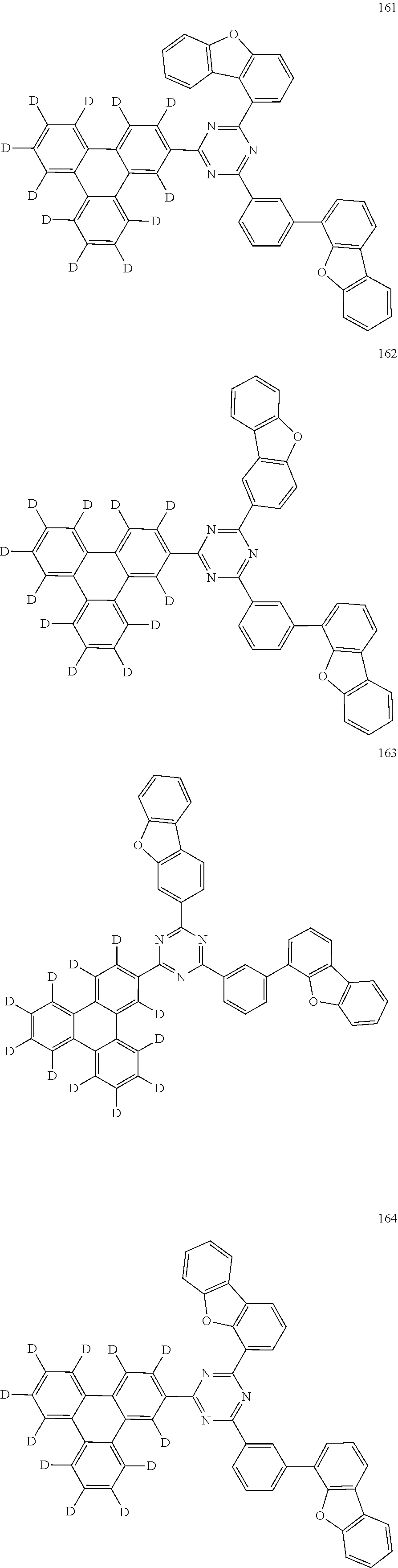

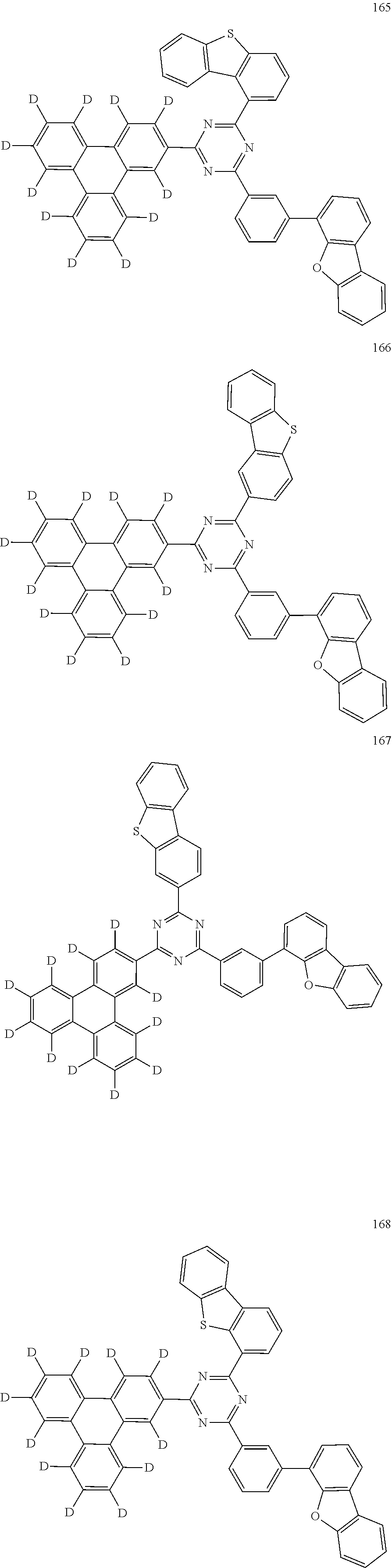

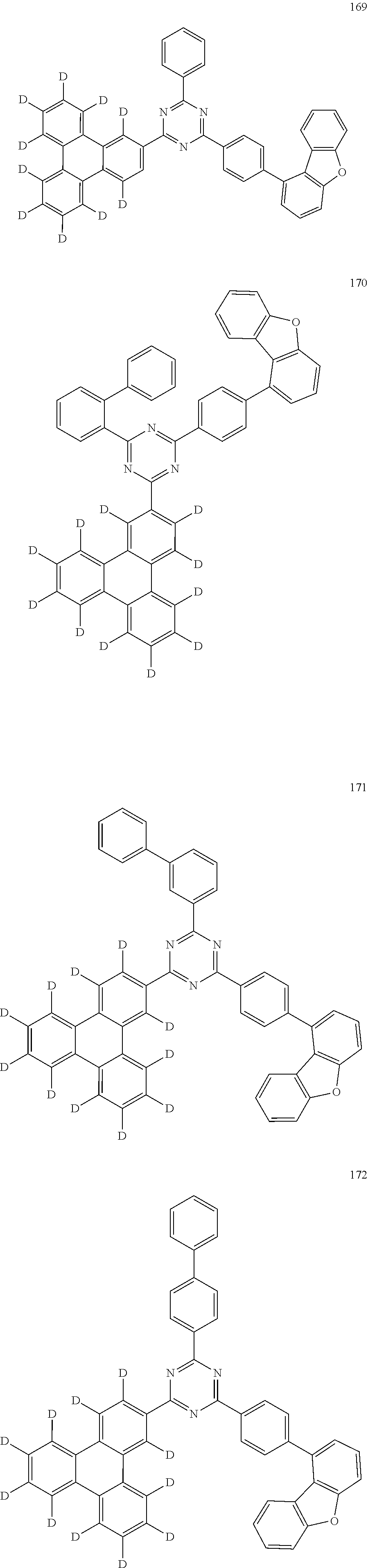

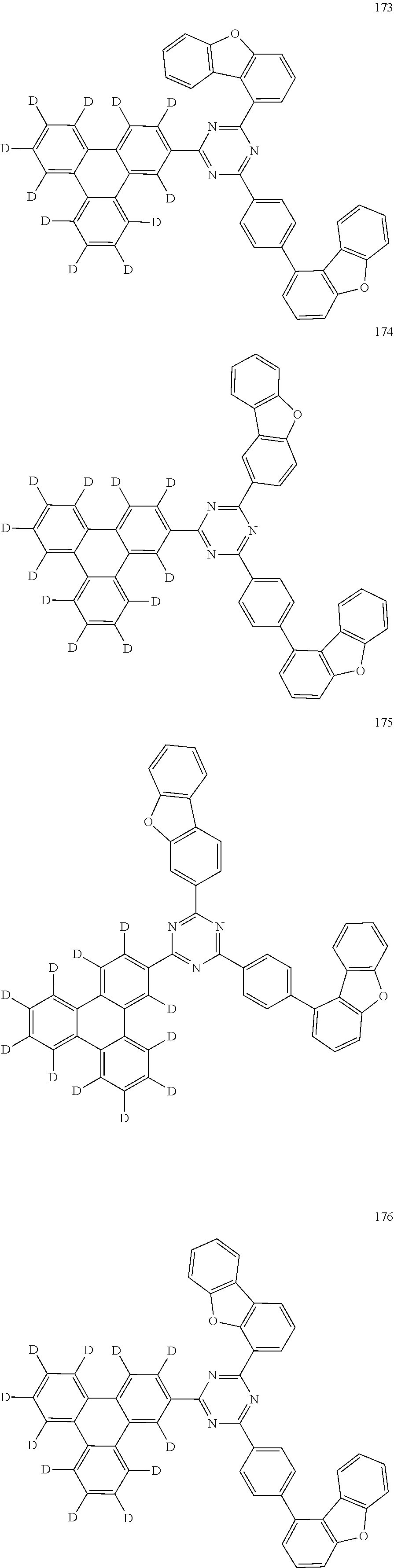

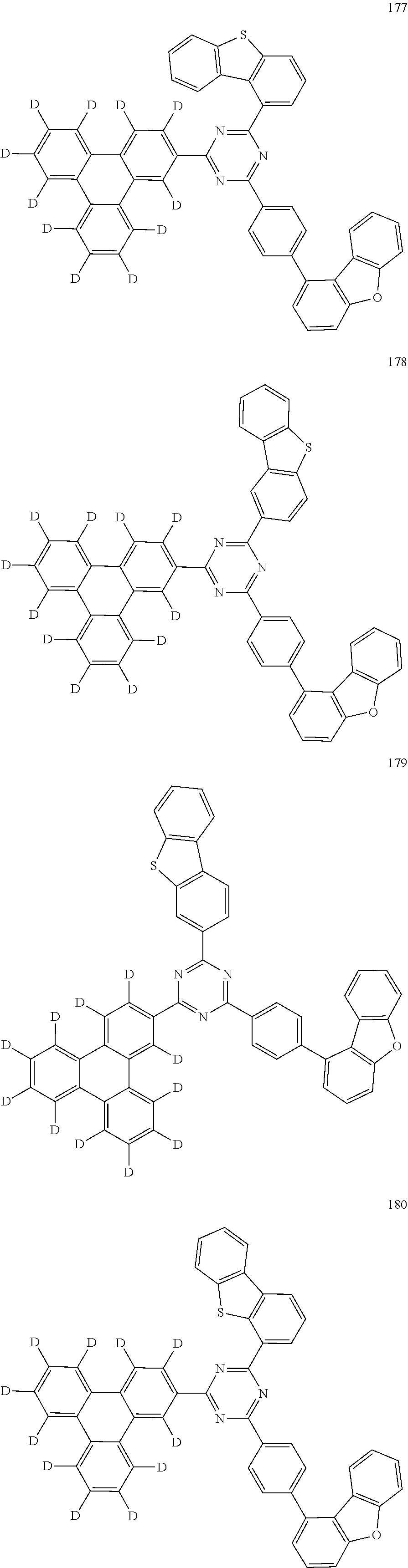

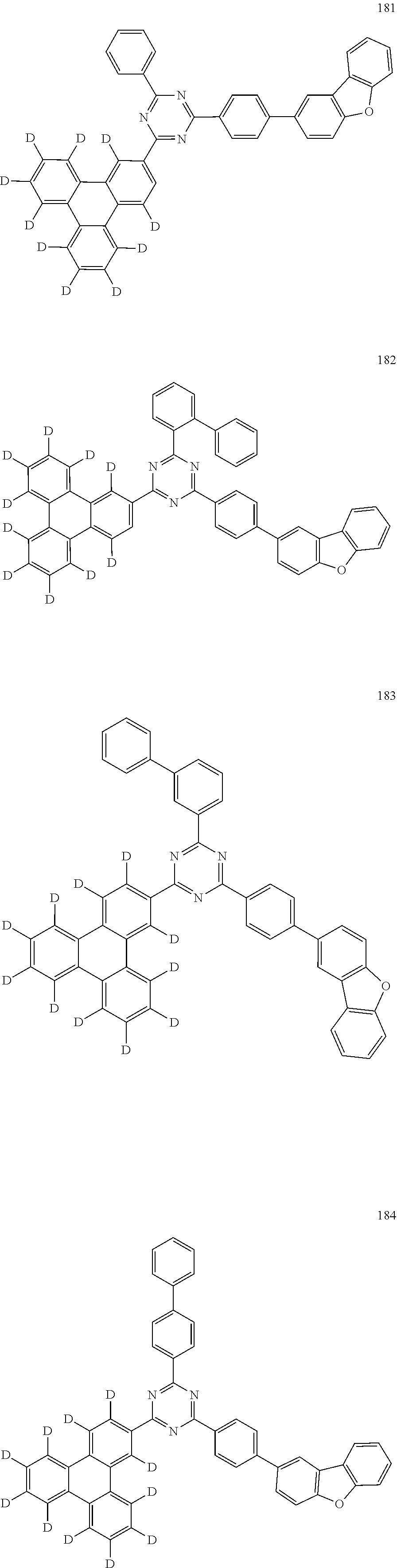

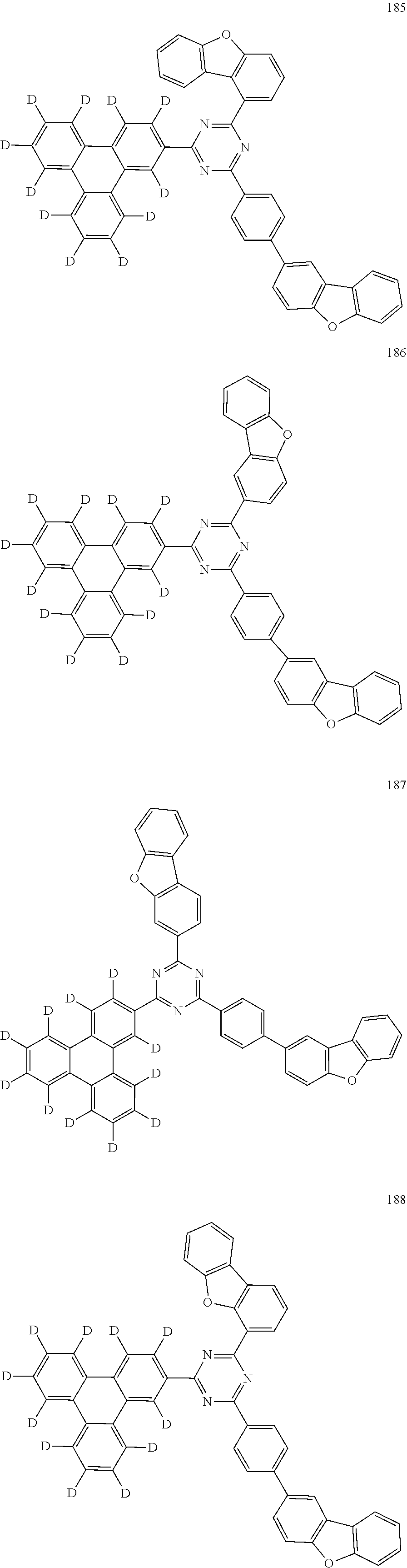

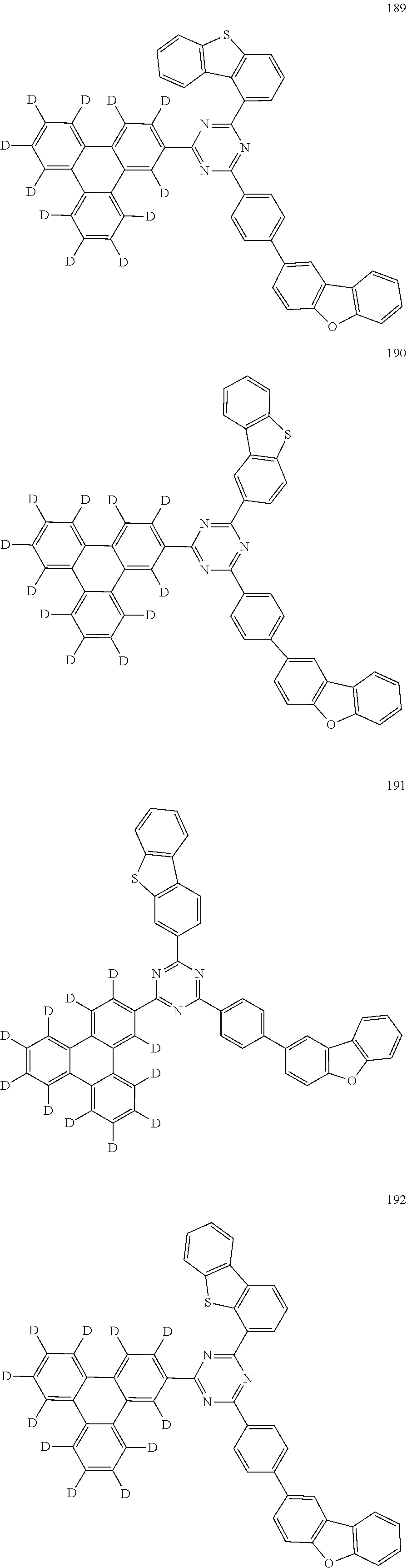

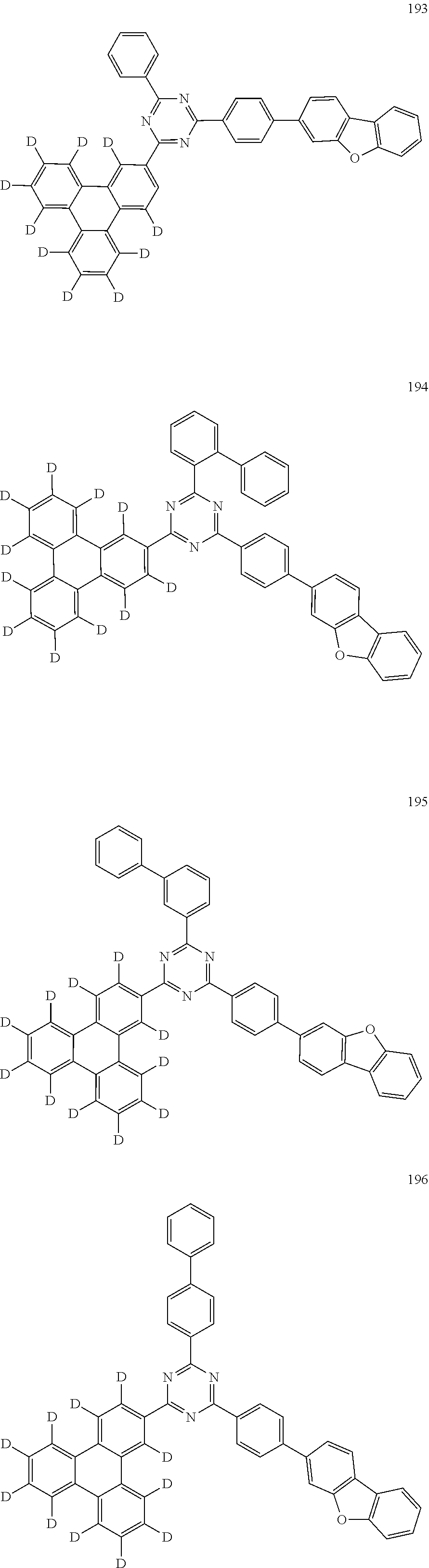

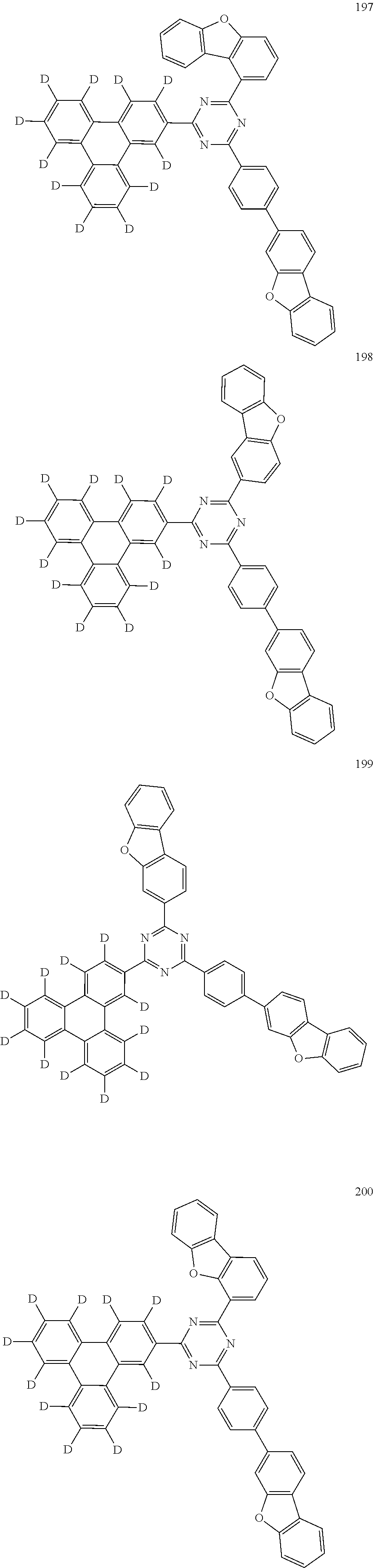

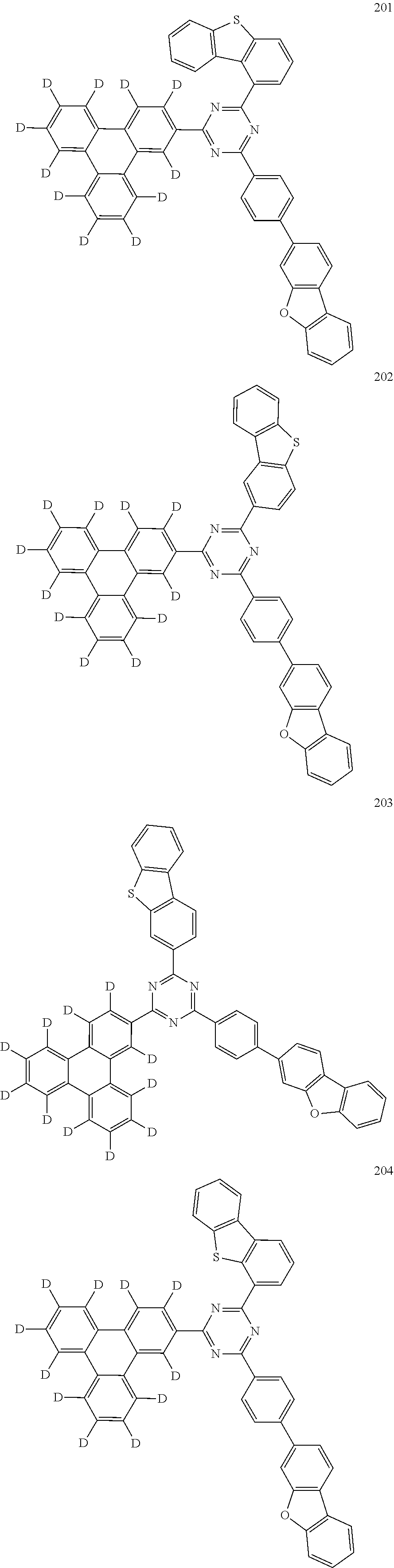

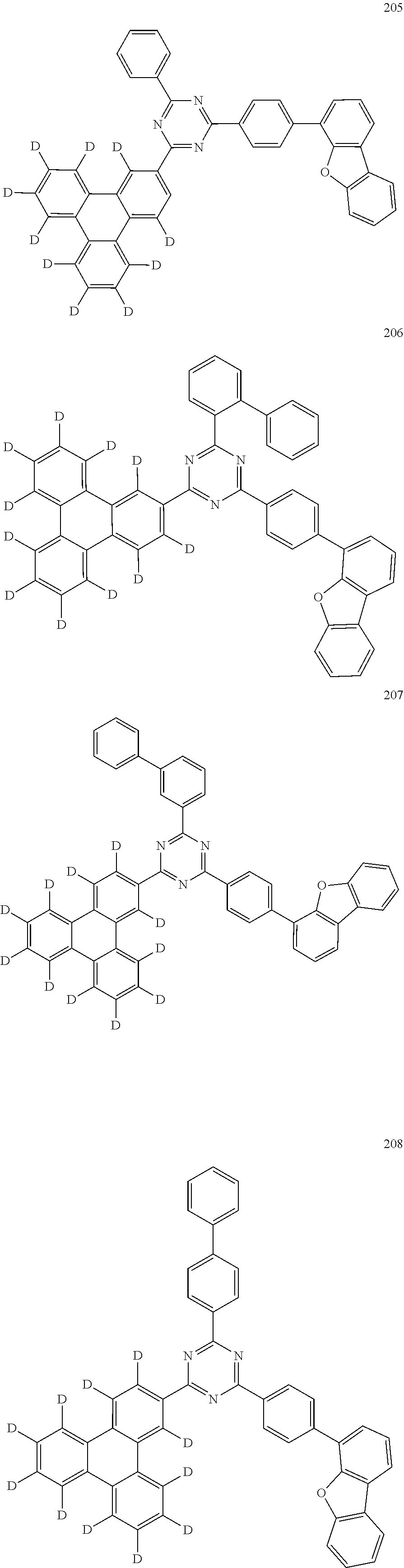

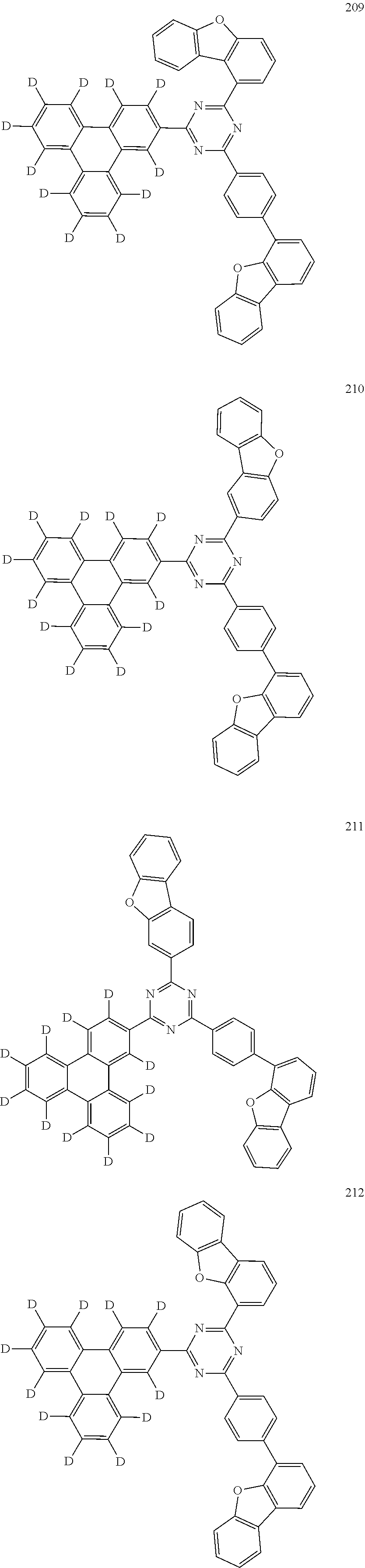

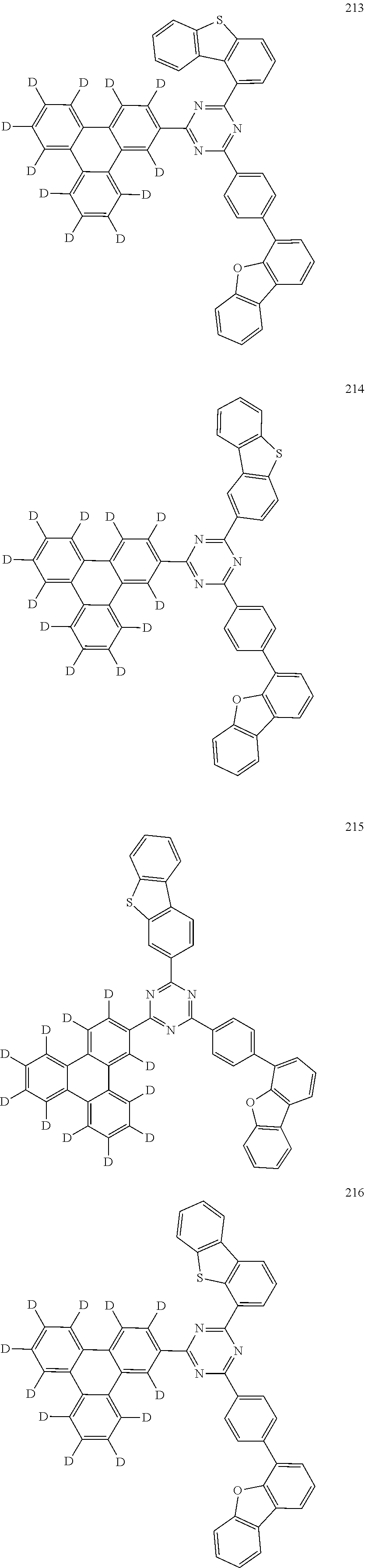

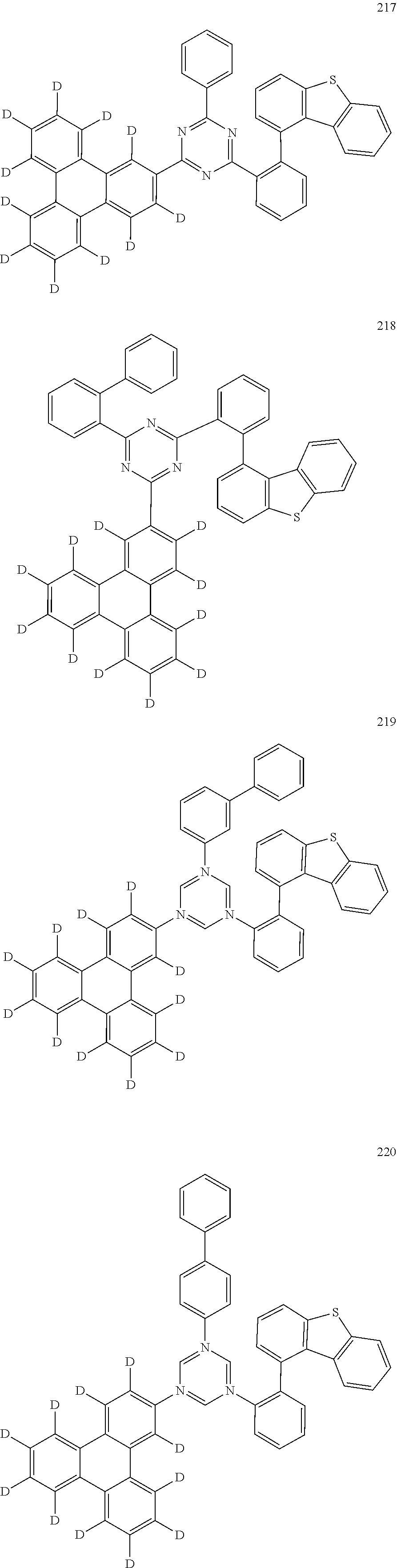

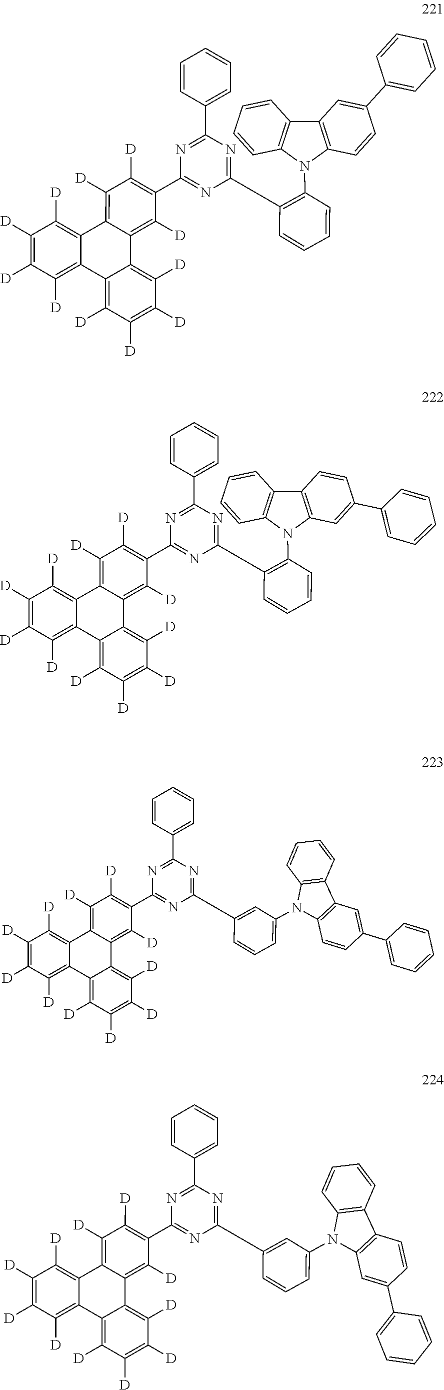

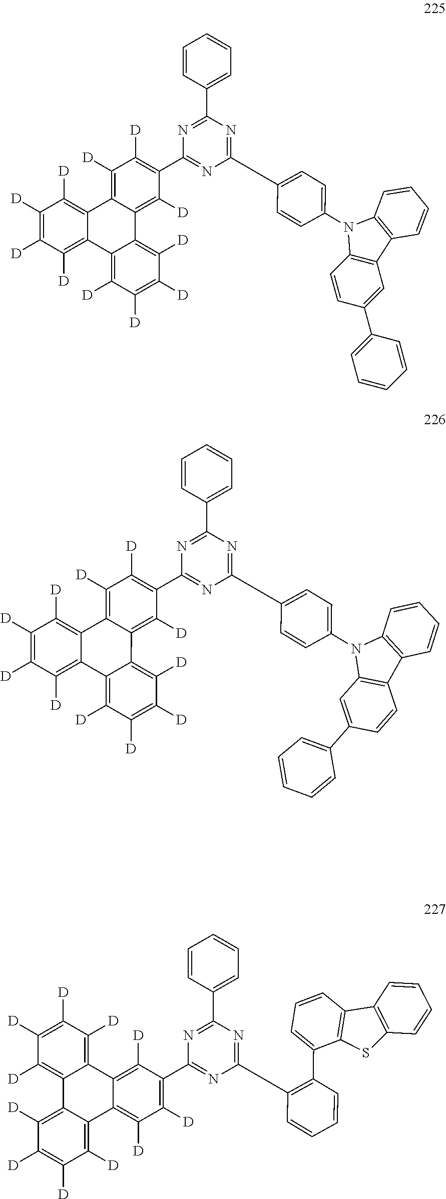 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-n5c6ccccc6c6cc(-c7ccccc7)ccc65)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccc(-n5c6ccccc6c6ccc(-c7ccccc7)cc65)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4sc4ccccc45)n3)c([2H])c([2H])c21]

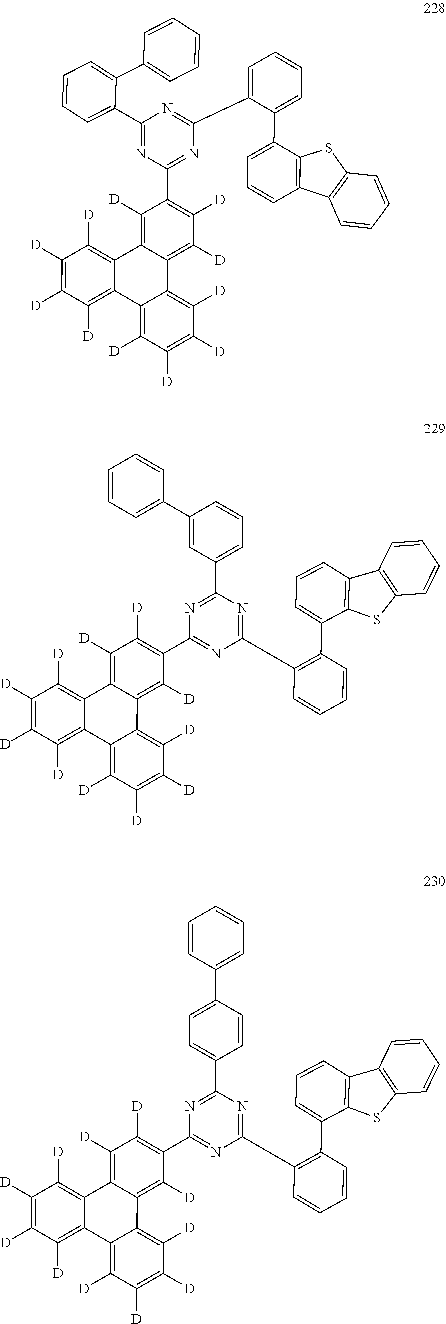 [SMILES: [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c4sc4ccccc45)n3)c([2H])c([2H])c21]

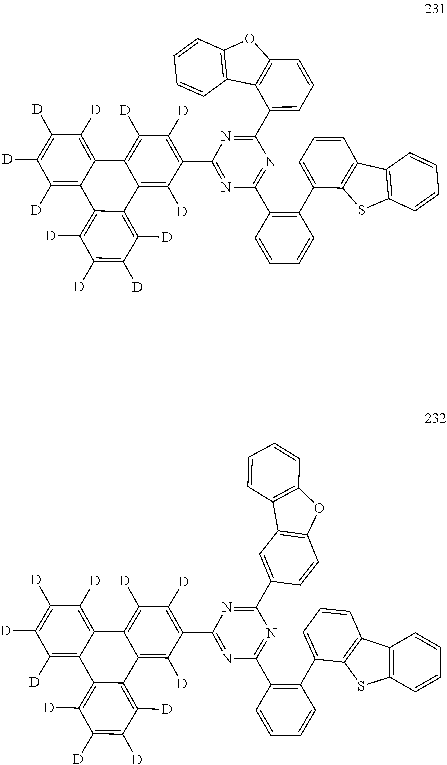 [SMILES: [2H]c1c([2H])c([2H])c2c(c1[2H])c1c([2H])c([2H])c([2H])c([2H])c1c1c([2H])c(-c3nc(-c4ccccc4-c4cccc5c4sc4ccccc45)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5c4sc4ccccc45)n3)c([2H])c([2H])c21]

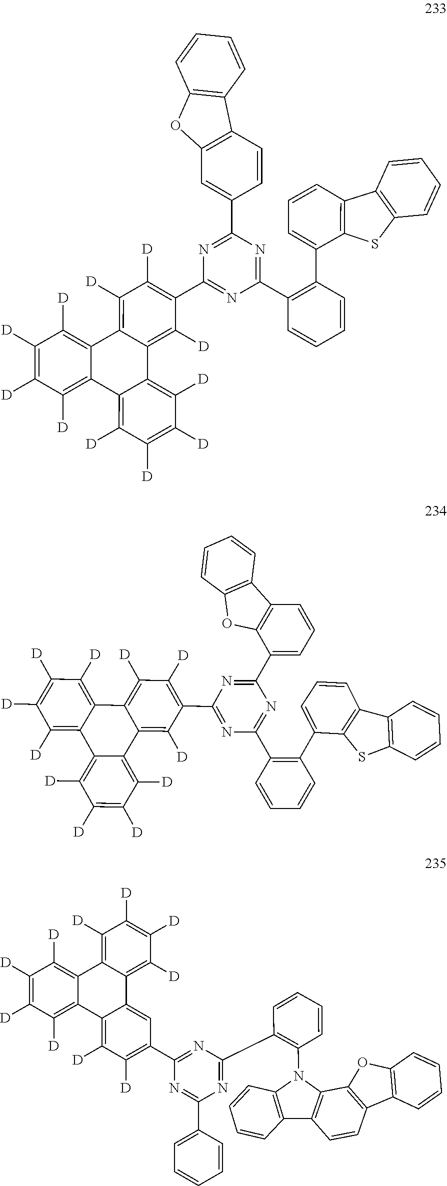 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c4sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4sc4ccccc45)nc(-c4cccc5c4oc4ccccc45)n3)c([2H])c([2H])c21 ; [2H]c1c(-c2nc(-c3ccccc3)nc(-c3ccccc3-n3c4ccccc4c4ccc5c6ccccc6oc5c43)n2)cc2c(c1[2H])c1c([2H])c([2H])c([2H])c([2H])c1c1c([2H])c([2H])c([2H])c([2H])c21]

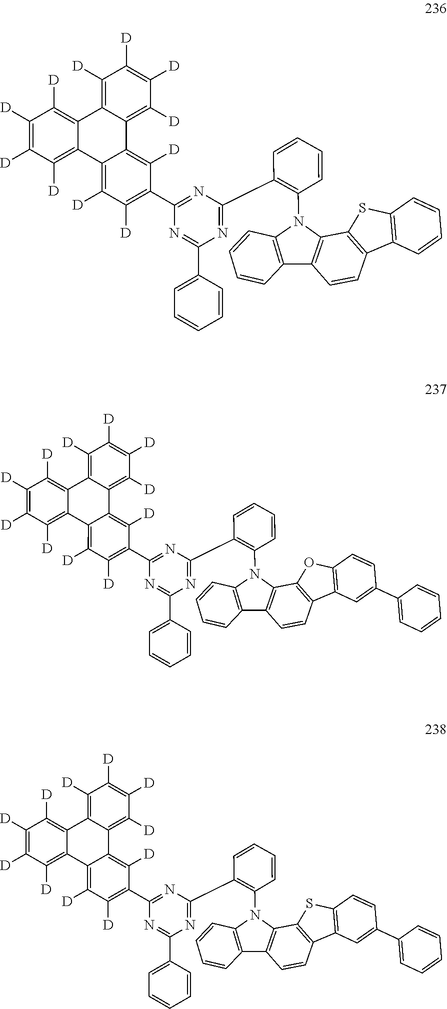 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7ccccc7sc6c54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7cc(-c8ccccc8)ccc7oc6c54)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4ccccc4-n4c5ccccc5c5ccc6c7cc(-c8ccccc8)ccc7sc6c54)n3)c([2H])c([2H])c21]

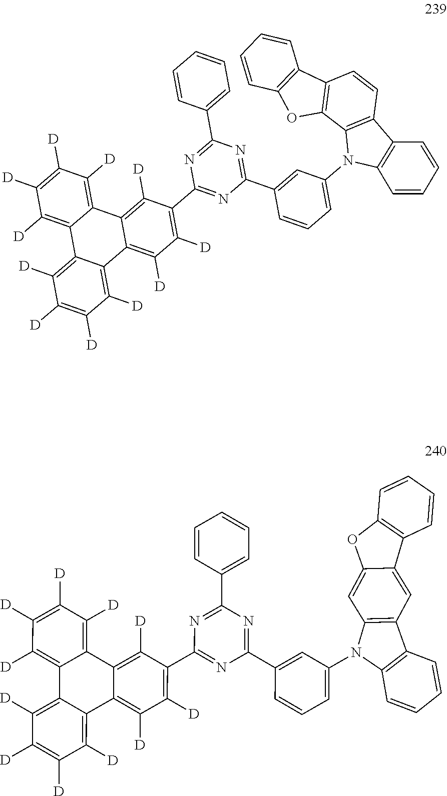 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8oc7c65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7c(cc65)oc5ccccc57)c4)n3)c([2H])c([2H])c21]

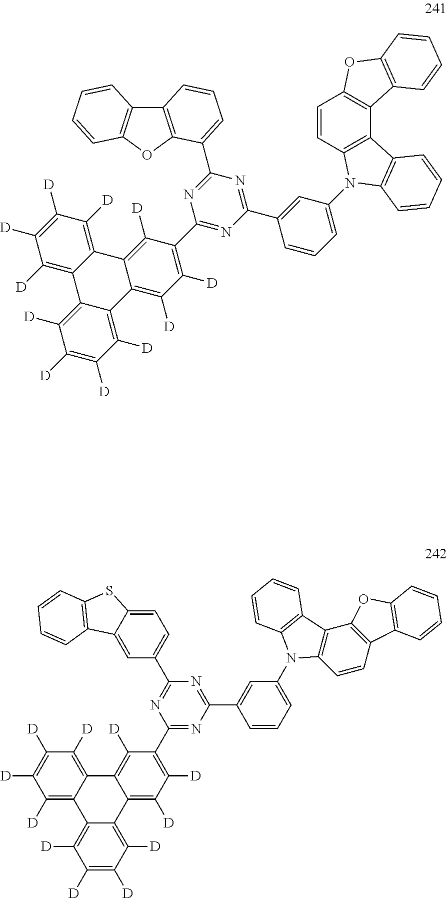 [SMILES: [2H]c1c([2H])c([2H])c2c(c1[2H])c1c([2H])c([2H])c([2H])c([2H])c1c1c([2H])c(-c3nc(-c4cccc(-n5c6ccccc6c6c7c(ccc65)oc5ccccc57)c4)nc(-c4cccc5c4oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6c7oc8ccccc8c7ccc65)c4)nc(-c4ccc5sc6ccccc6c5c4)n3)c([2H])c([2H])c21]

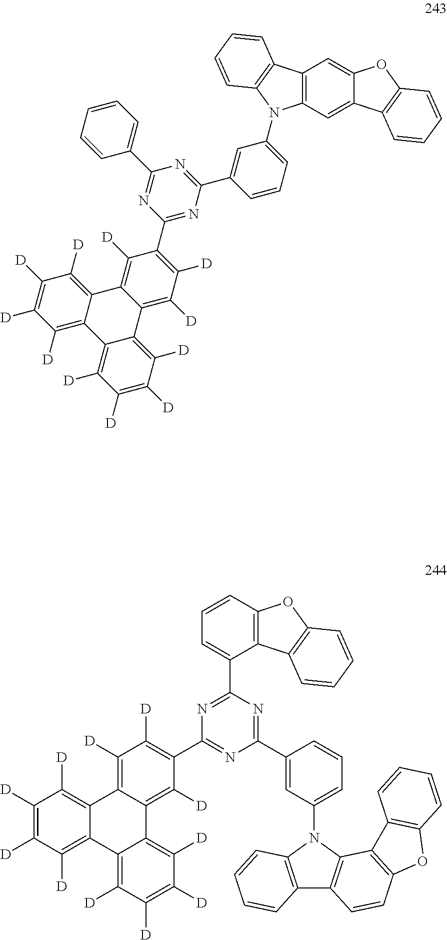 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7oc8ccccc8c7cc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-n5c6ccccc6c6ccc7oc8ccccc8c7c65)c4)nc(-c4cccc5oc6ccccc6c45)n3)c([2H])c([2H])c21]

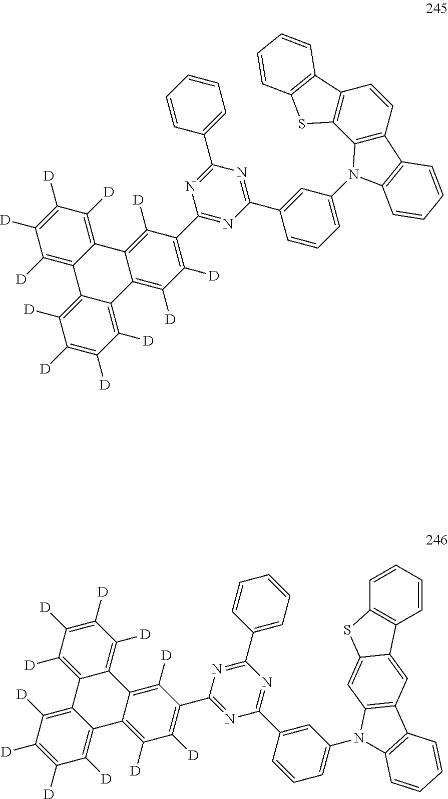 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8sc7c65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7c(cc65)sc5ccccc57)c4)n3)c([2H])c([2H])c21]

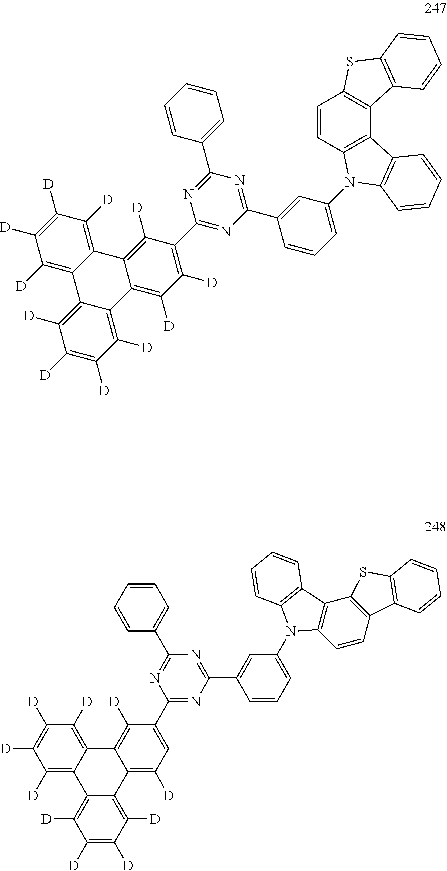 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6c7c(ccc65)sc5ccccc57)c4)n3)c([2H])c([2H])c21 ; [2H]c1c([2H])c([2H])c2c(c1[2H])c1c([2H])cc(-c3nc(-c4ccccc4)nc(-c4cccc(-n5c6ccccc6c6c7sc8ccccc8c7ccc65)c4)n3)c([2H])c1c1c([2H])c([2H])c([2H])c([2H])c12]

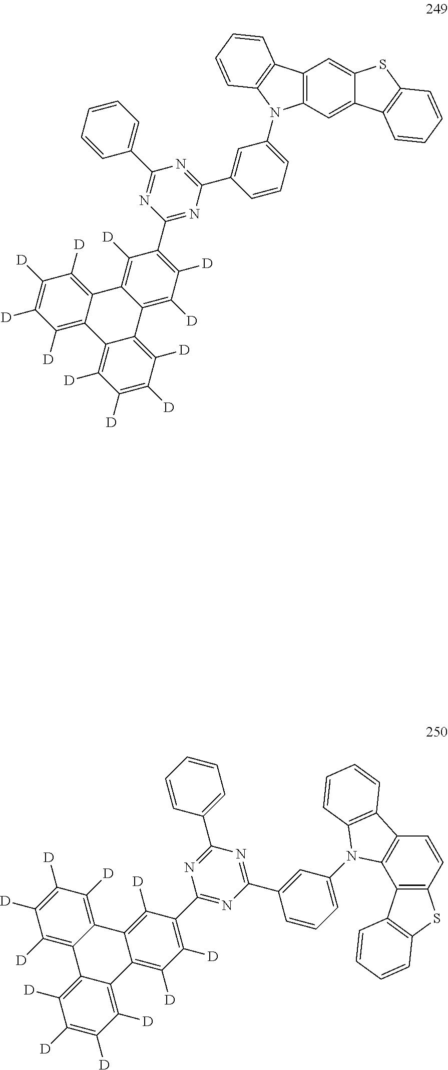 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6cc7sc8ccccc8c7cc65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7sc8ccccc8c7c65)c4)n3)c([2H])c([2H])c21]

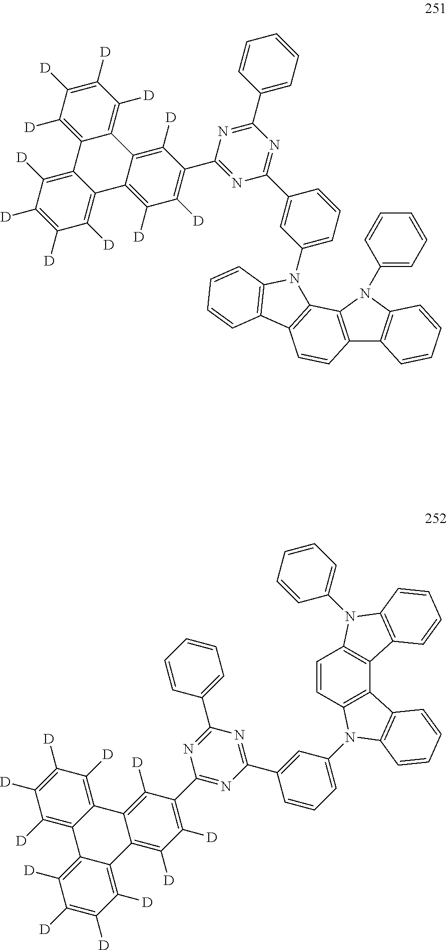 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6ccc7c8ccccc8n(-c8ccccc8)c7c65)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4)nc(-c4cccc(-n5c6ccccc6c6c7c8ccccc8n(-c8ccccc8)c7ccc65)c4)n3)c([2H])c([2H])c21]

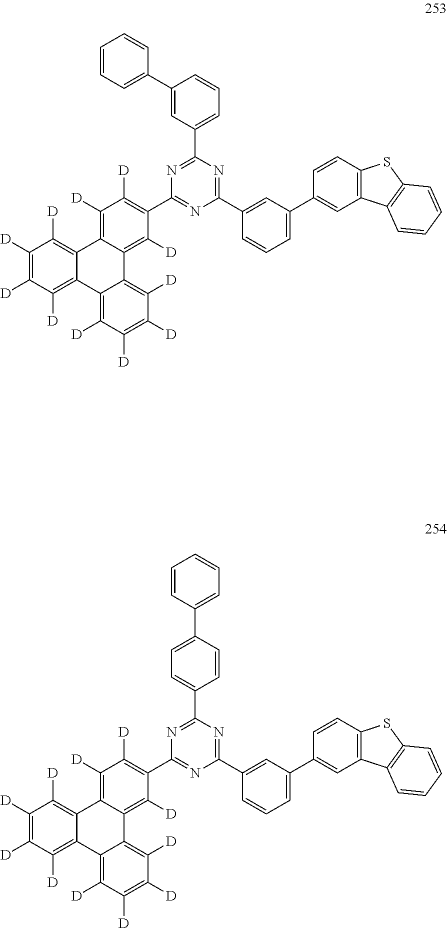 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6sc7ccccc7c6c5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6sc7ccccc7c6c5)c4)n3)c([2H])c([2H])c21]

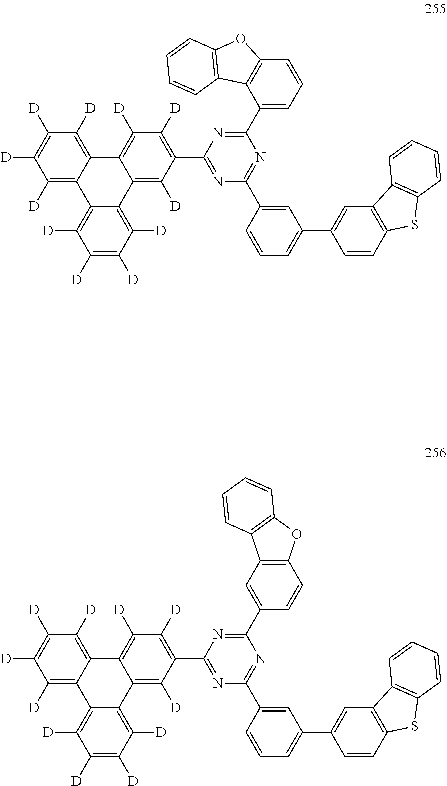 [SMILES: [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4ccc5oc6ccccc6c5c4)n3)c([2H])c([2H])c21]

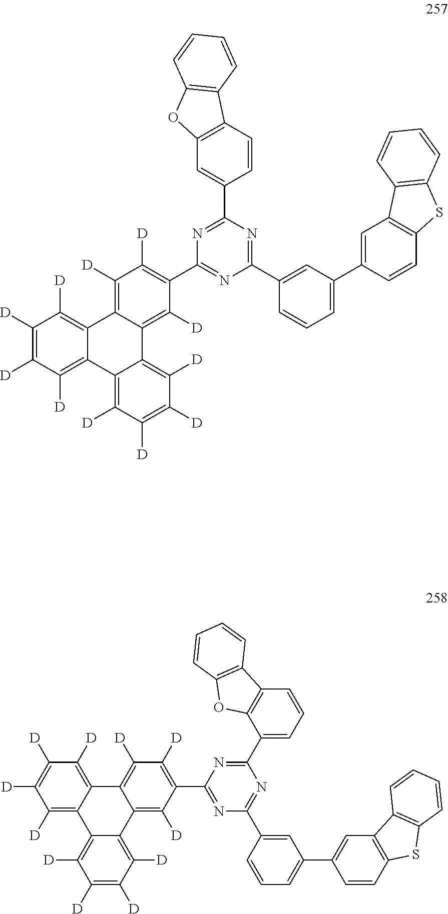 [SMILES: [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4cccc5c4oc4ccccc45)n3)c([2H])c([2H])c21]

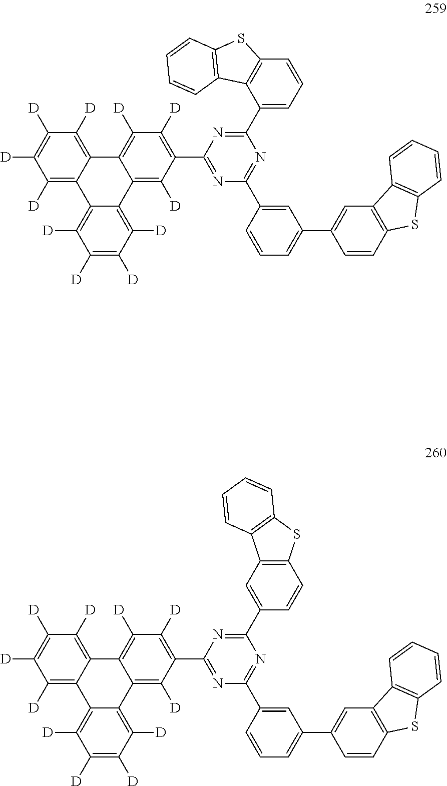 [SMILES: [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4ccc5sc6ccccc6c5c4)n3)c([2H])c([2H])c21]

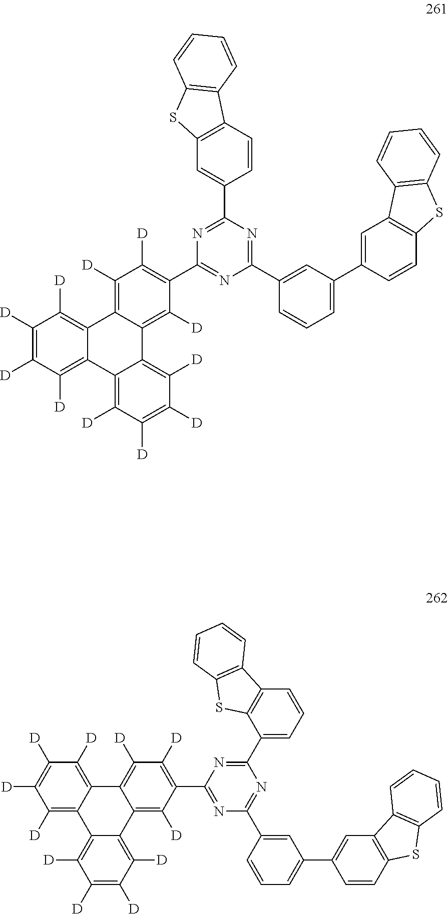 [SMILES: [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6sc7ccccc7c6c5)c4)nc(-c4cccc5c4sc4ccccc45)n3)c([2H])c([2H])c21]

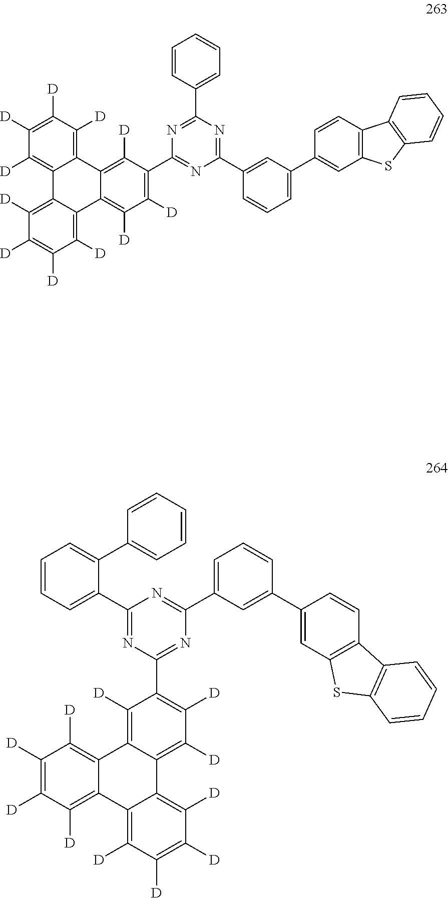 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6c(c5)sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4ccccc4-c4ccccc4)n3)c([2H])c([2H])c21]

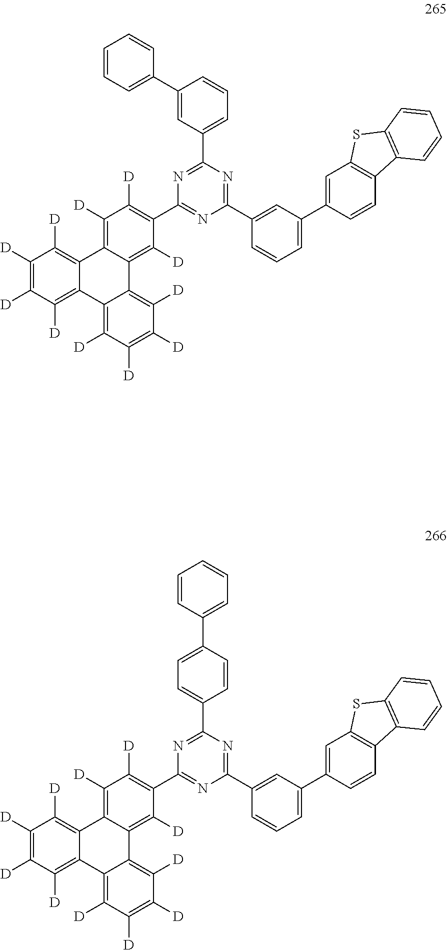 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6c(c5)sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6c(c5)sc5ccccc56)c4)n3)c([2H])c([2H])c21]

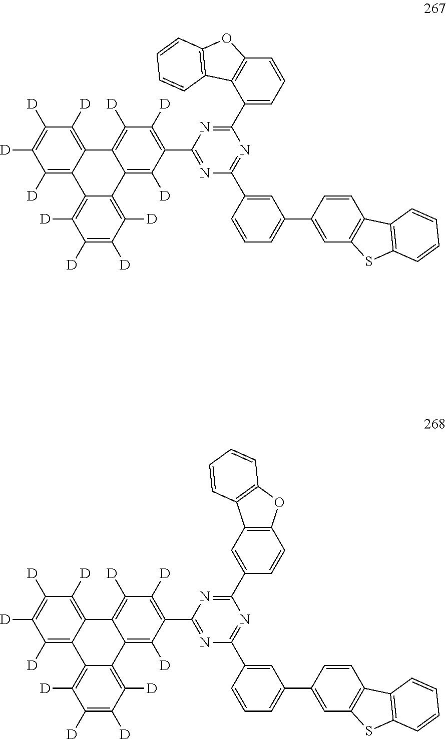 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4ccc5oc6ccccc6c5c4)n3)c([2H])c([2H])c21]

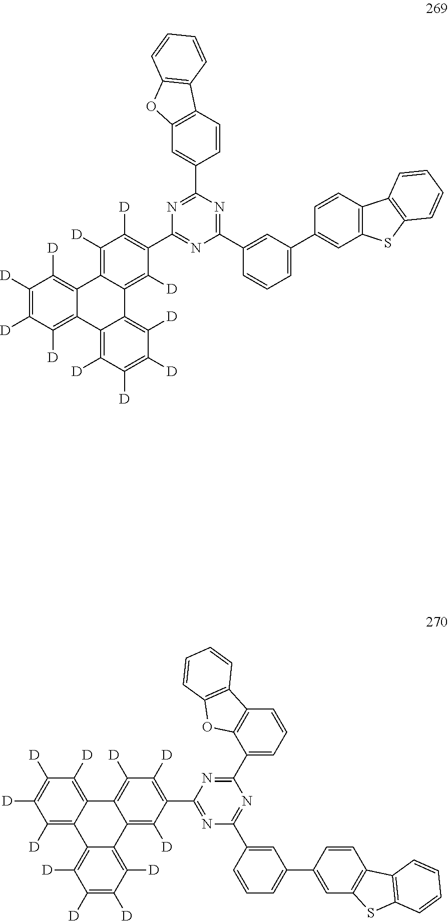 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4cccc5c4oc4ccccc45)n3)c([2H])c([2H])c21]

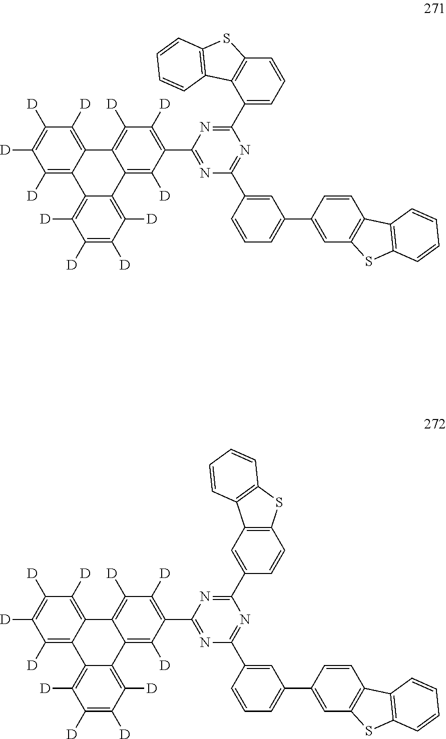 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4ccc5sc6ccccc6c5c4)n3)c([2H])c([2H])c21]

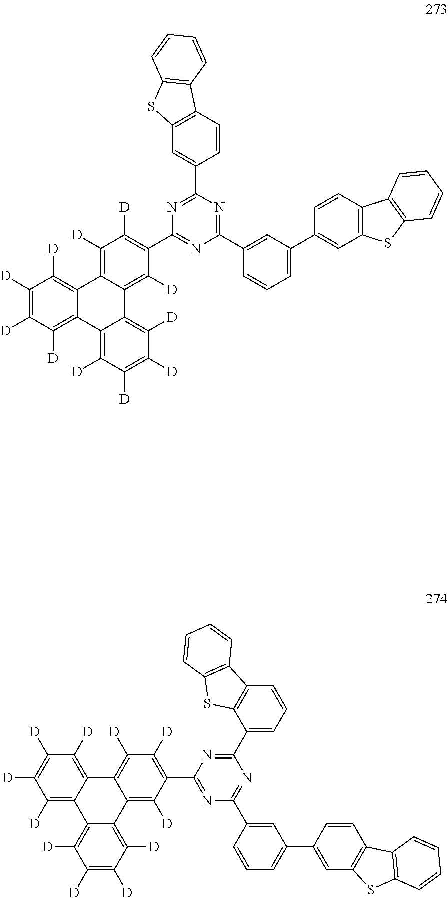 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)sc5ccccc56)c4)nc(-c4cccc5c4sc4ccccc45)n3)c([2H])c([2H])c21]

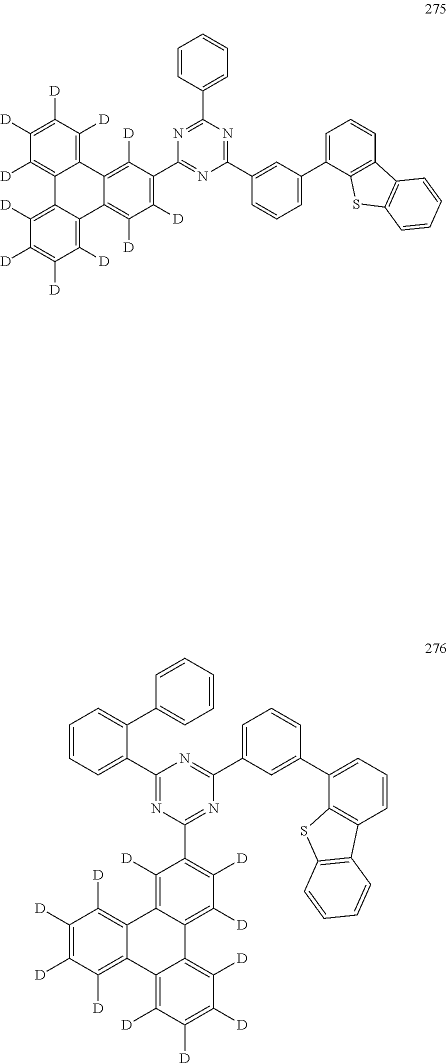 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c5sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccccc4-c4ccccc4)n3)c([2H])c([2H])c21]

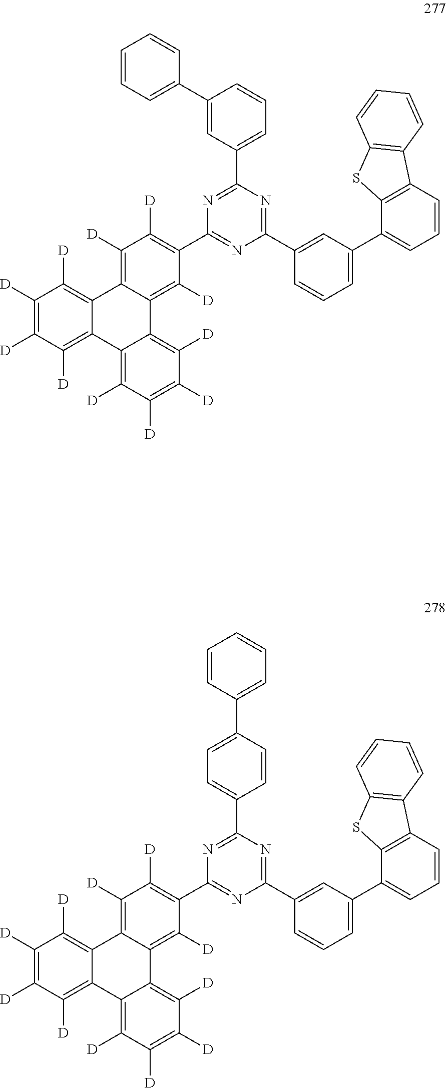 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c5sc5ccccc56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5cccc6c5sc5ccccc56)c4)n3)c([2H])c([2H])c21]

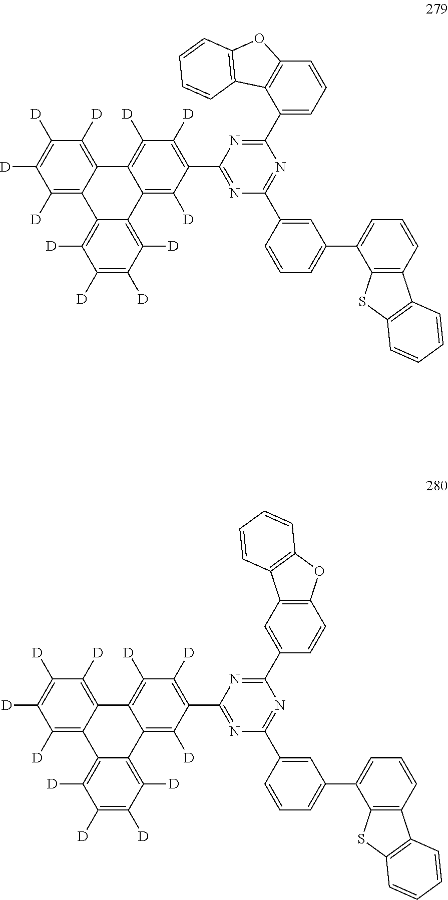 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5oc6ccccc6c5c4)n3)c([2H])c([2H])c21]

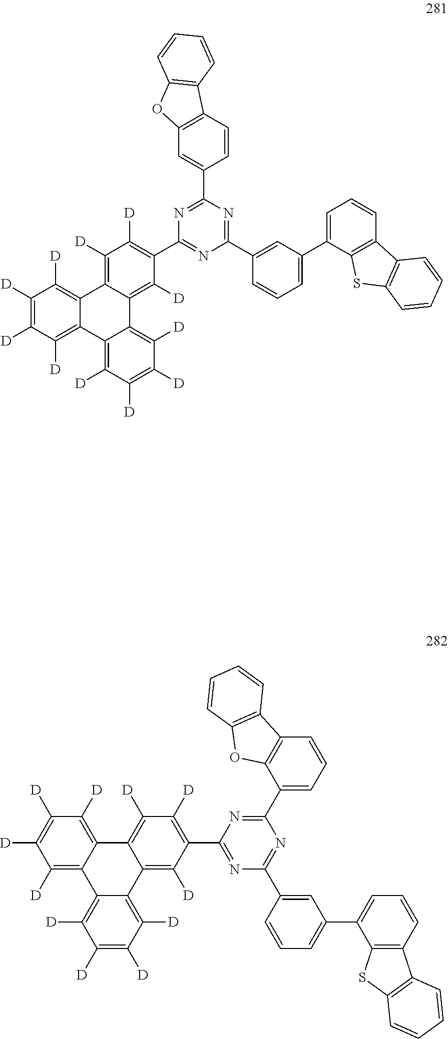 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5c4oc4ccccc45)n3)c([2H])c([2H])c21]

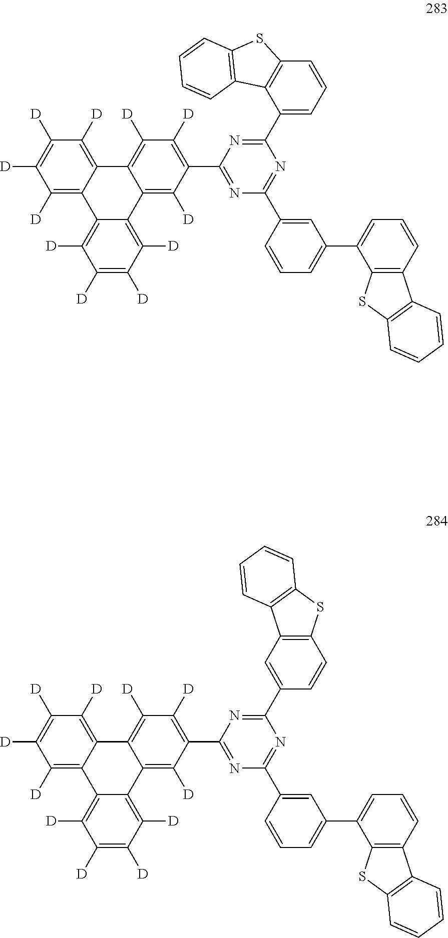 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5sc6ccccc6c5c4)n3)c([2H])c([2H])c21]

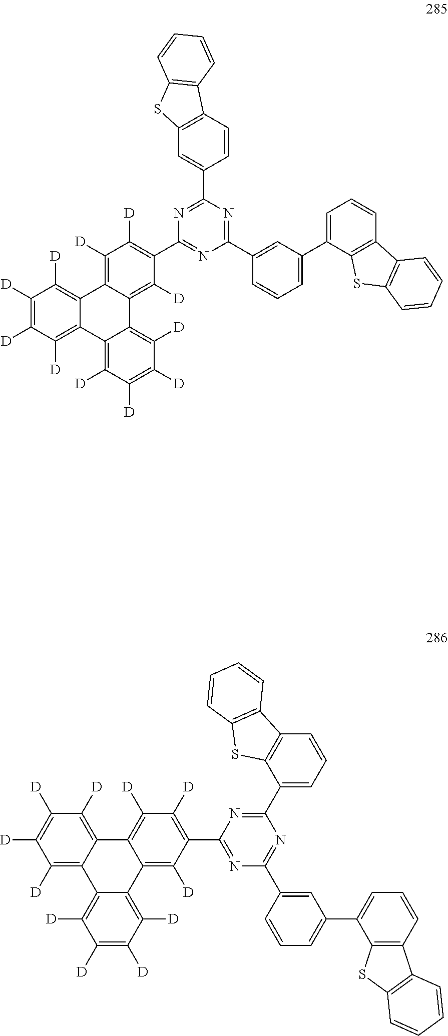 [SMILES: [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5sc5ccccc56)c4)nc(-c4cccc5c4sc4ccccc45)n3)c([2H])c([2H])c21]

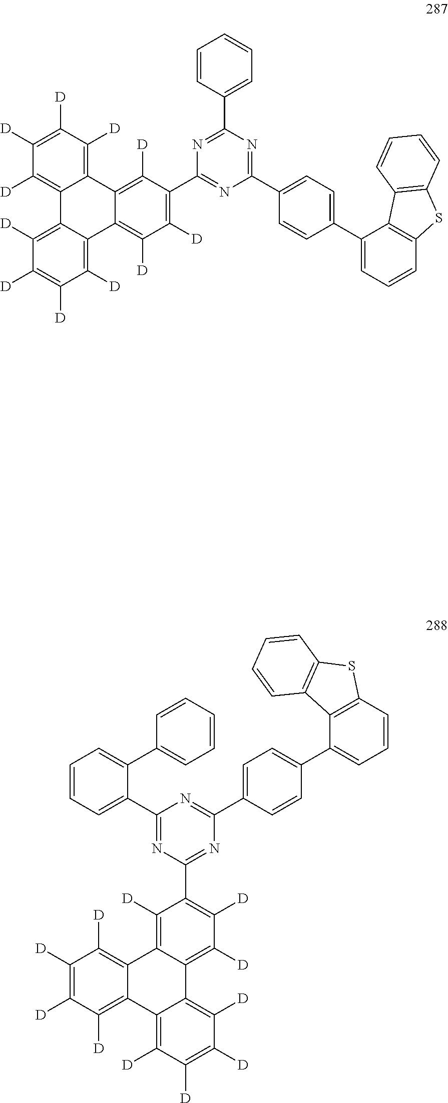 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6sc7ccccc7c56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccccc4-c4ccccc4)n3)c([2H])c([2H])c21]

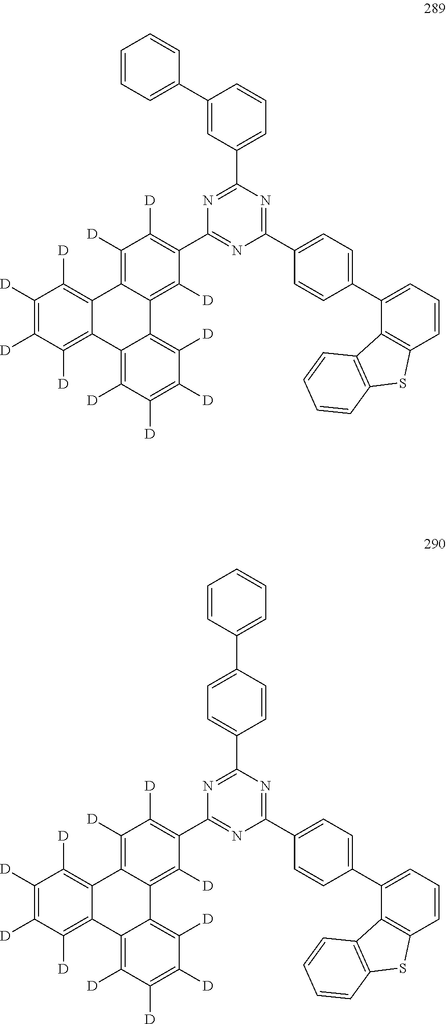 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6sc7ccccc7c56)cc4)n3)c([2H])c([2H])c21]

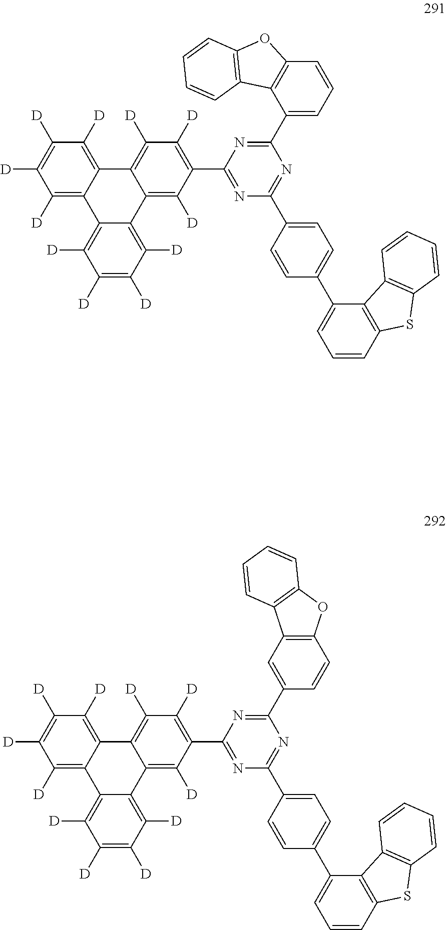 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5oc6ccccc6c5c4)n3)c([2H])c([2H])c21]

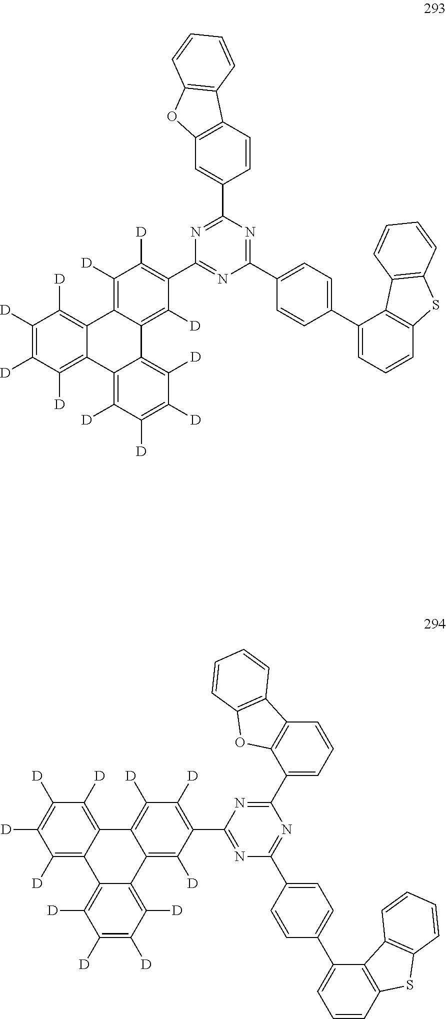 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5c4oc4ccccc45)n3)c([2H])c([2H])c21]

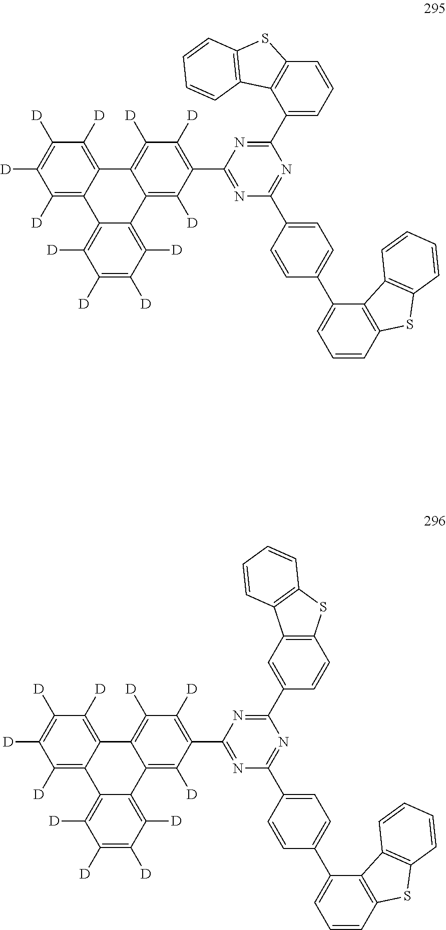 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5sc6ccccc6c5c4)n3)c([2H])c([2H])c21]

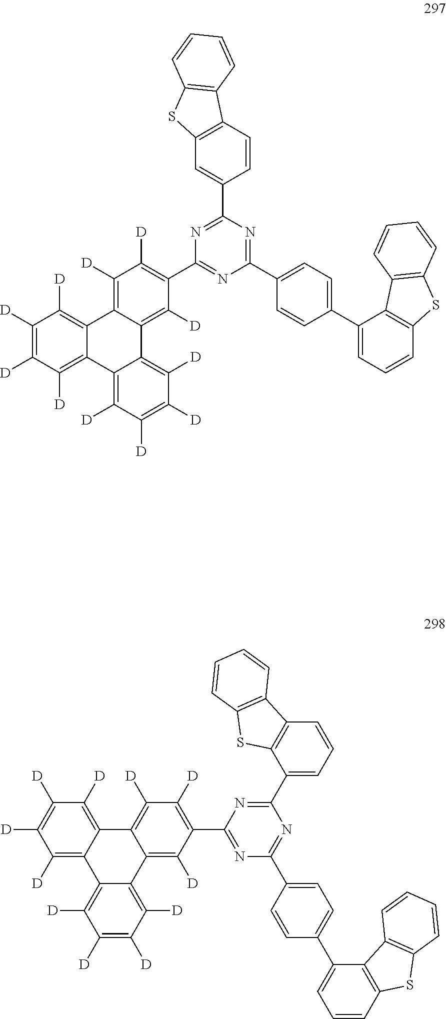 [SMILES: [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6sc7ccccc7c56)cc4)nc(-c4cccc5c4sc4ccccc45)n3)c([2H])c([2H])c21]

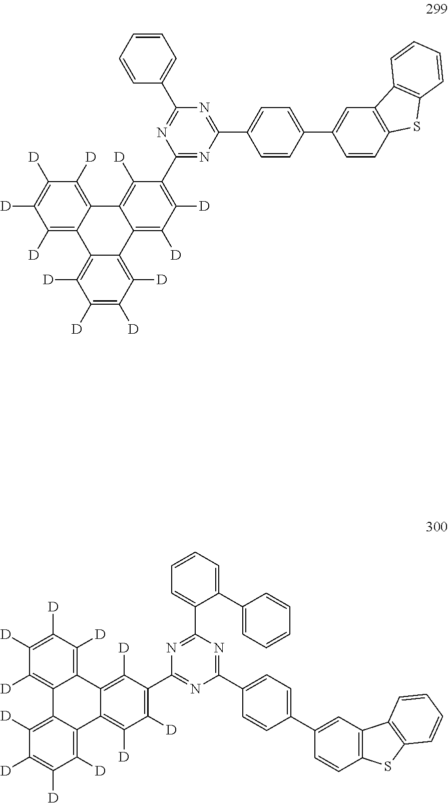 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5ccc6sc7ccccc7c6c5)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccccc4-c4ccccc4)n3)c([2H])c([2H])c21]

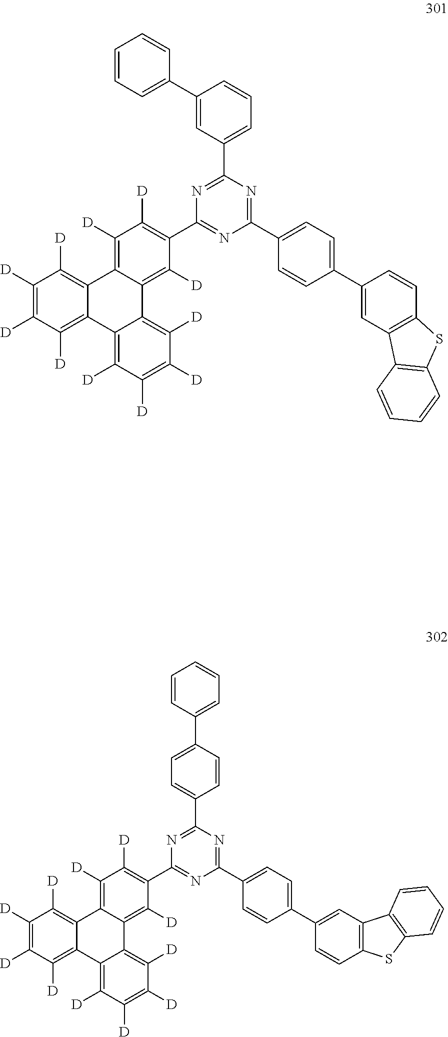 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5ccc6sc7ccccc7c6c5)cc4)n3)c([2H])c([2H])c21]

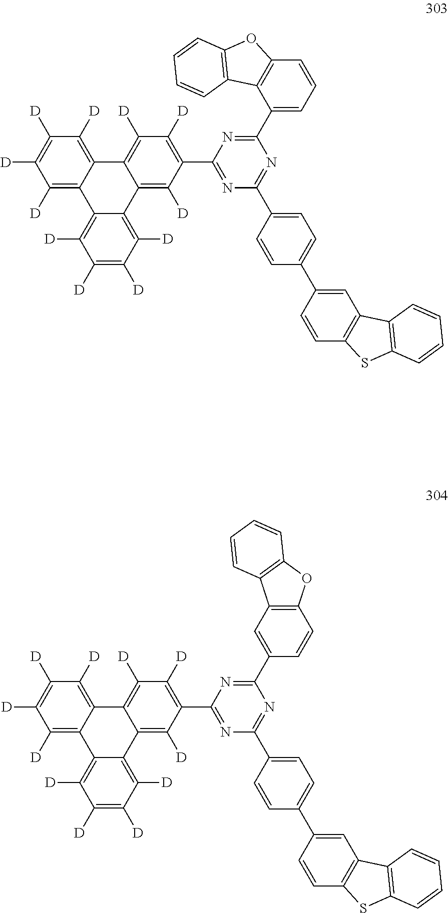 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5oc6ccccc6c5c4)n3)c([2H])c([2H])c21]

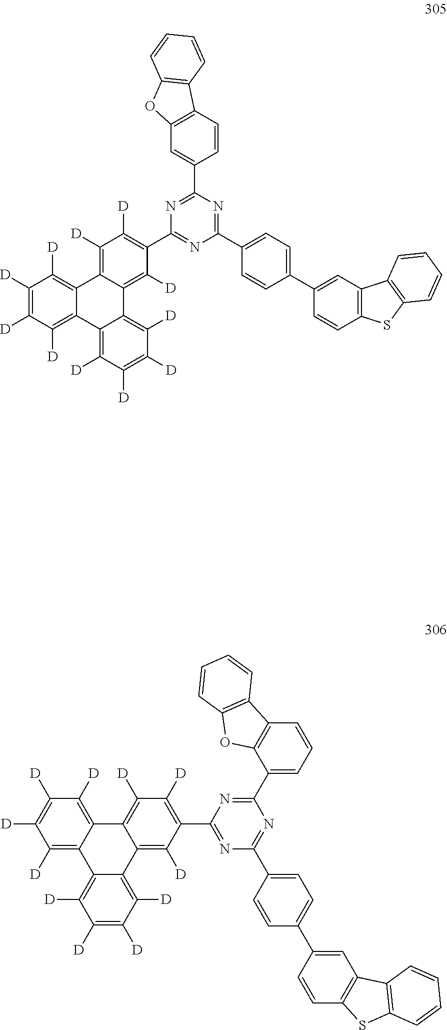 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5c4oc4ccccc45)n3)c([2H])c([2H])c21]

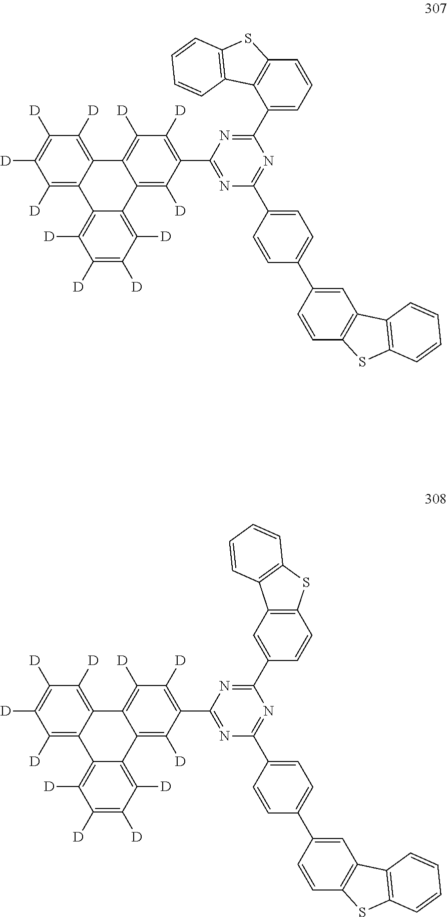 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5sc6ccccc6c5c4)n3)c([2H])c([2H])c21]

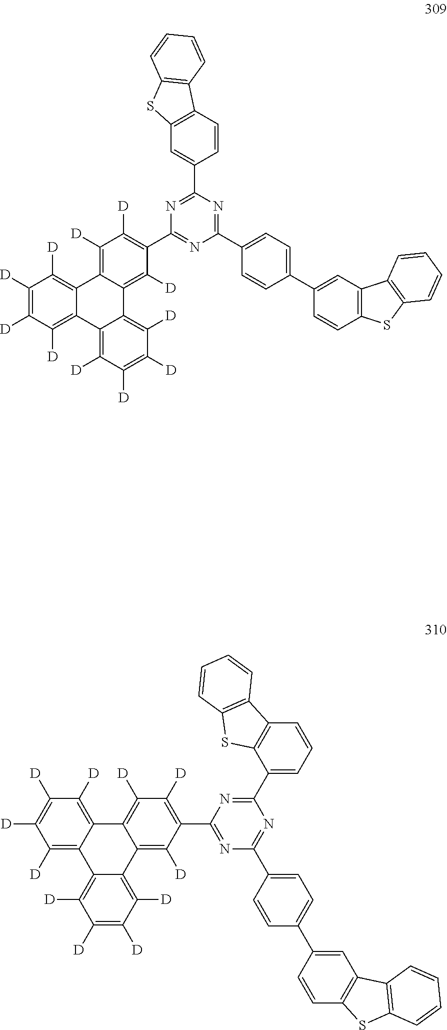 [SMILES: [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6sc7ccccc7c6c5)cc4)nc(-c4cccc5c4sc4ccccc45)n3)c([2H])c([2H])c21]

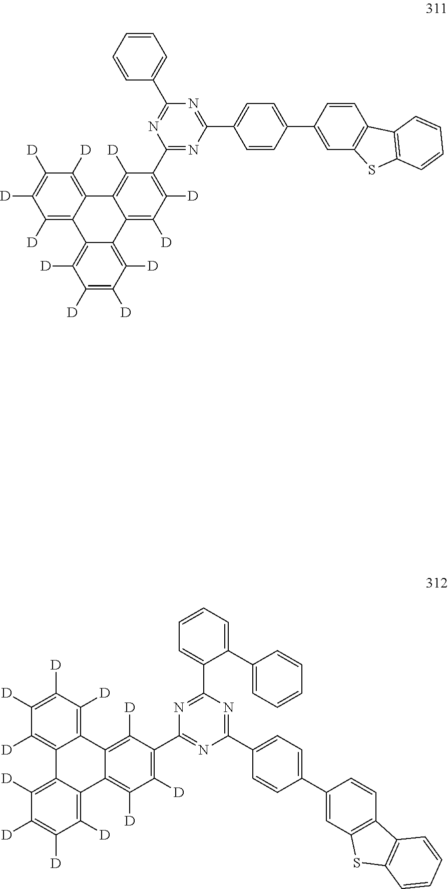 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5ccc6c(c5)sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccccc4-c4ccccc4)n3)c([2H])c([2H])c21]

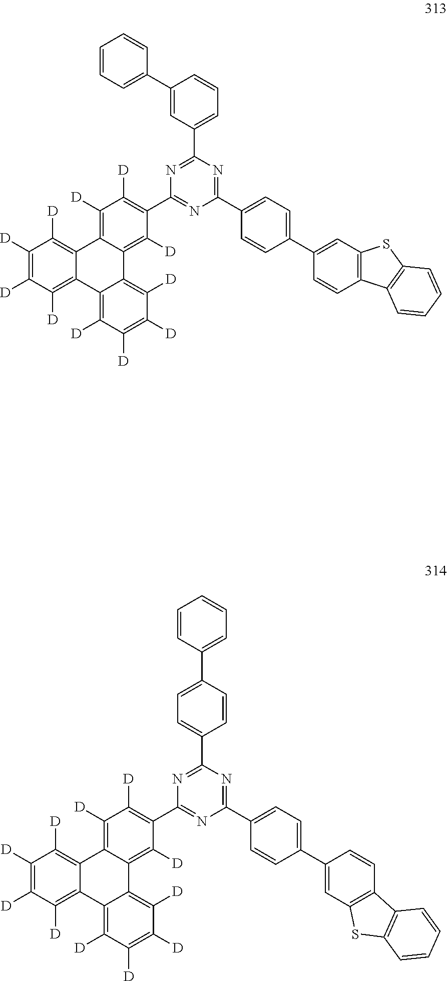 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5ccc6c(c5)sc5ccccc56)cc4)n3)c([2H])c([2H])c21]

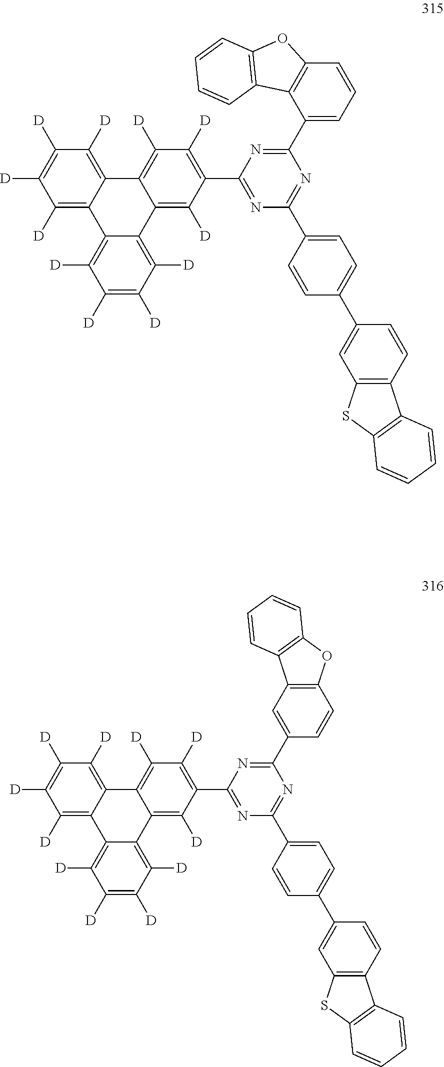 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5oc6ccccc6c5c4)n3)c([2H])c([2H])c21]

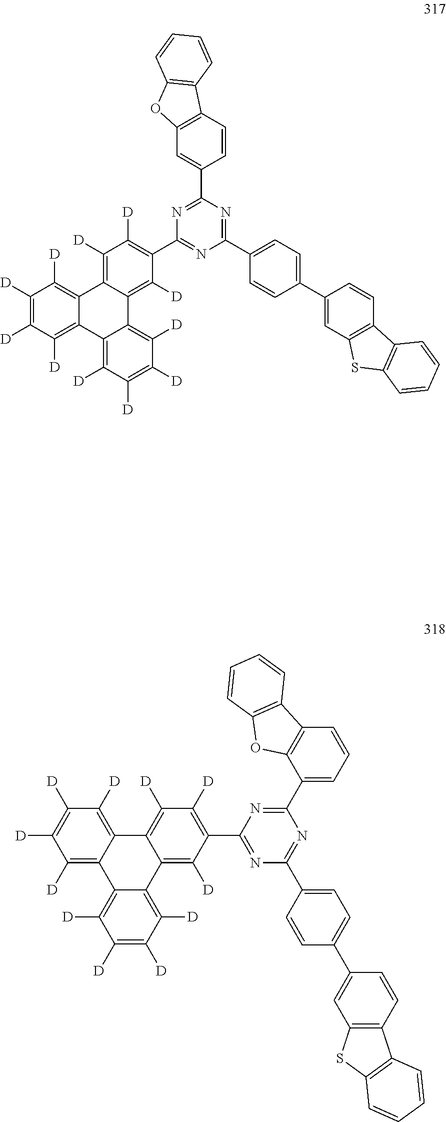 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4oc4ccccc45)n3)c([2H])c([2H])c21]

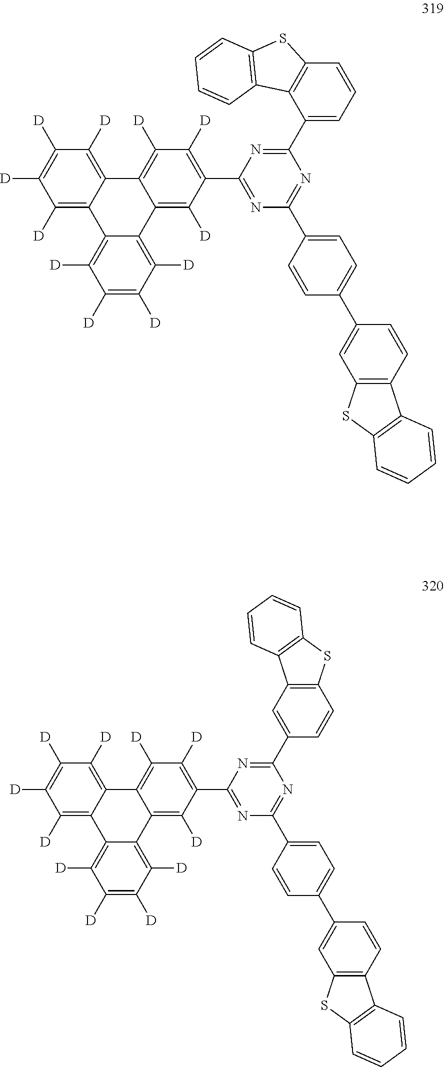 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5sc6ccccc6c5c4)n3)c([2H])c([2H])c21]

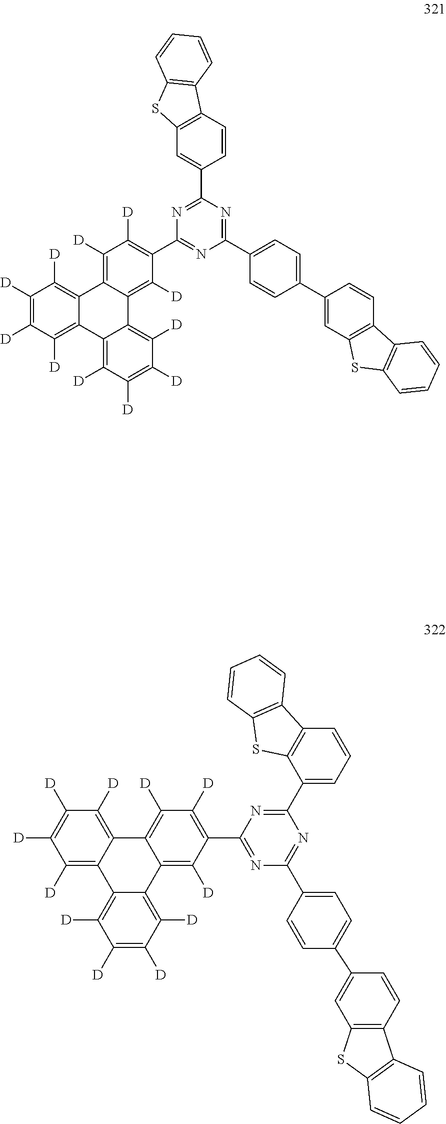 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4sc4ccccc45)n3)c([2H])c([2H])c21]

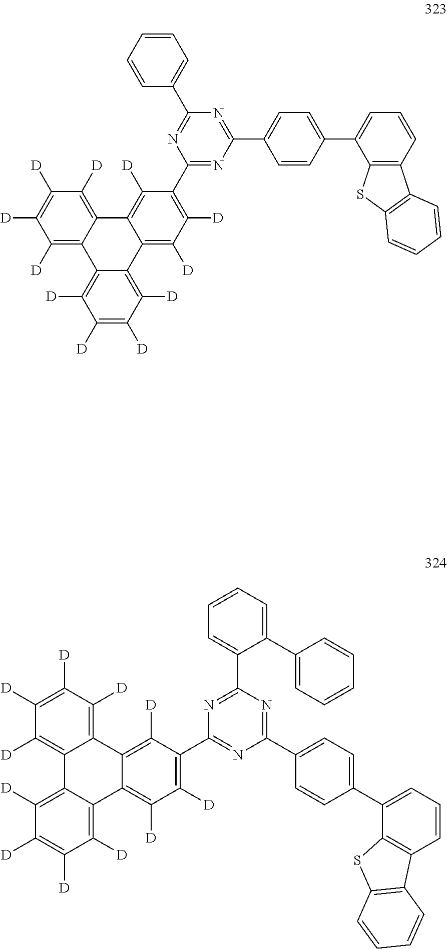 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccccc4-c4ccccc4)n3)c([2H])c([2H])c21]

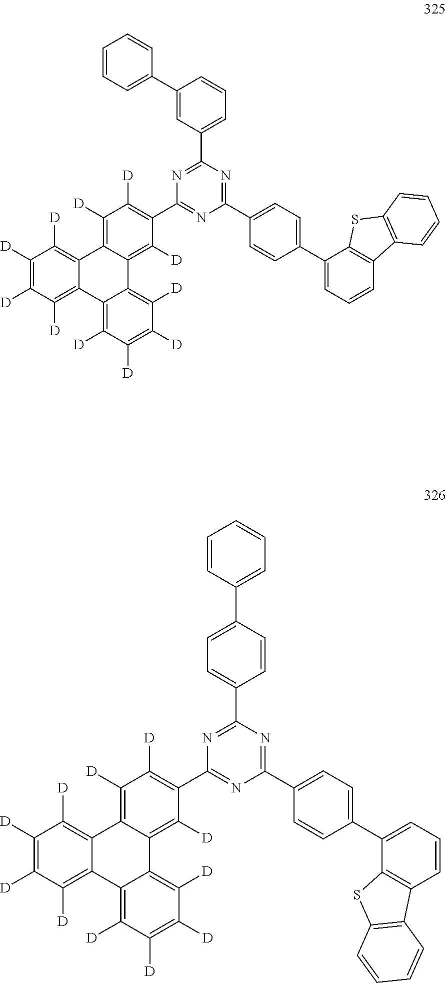 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6c5sc5ccccc56)cc4)n3)c([2H])c([2H])c21]

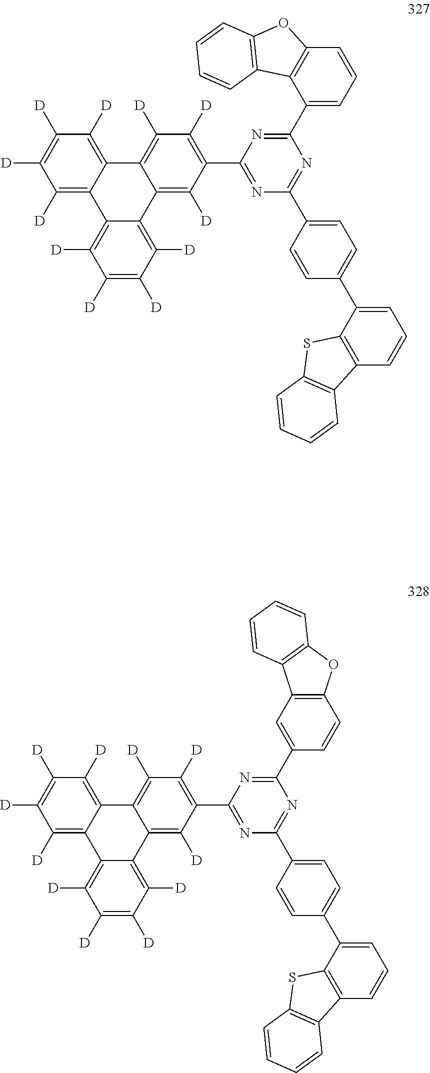 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5oc6ccccc6c5c4)n3)c([2H])c([2H])c21]

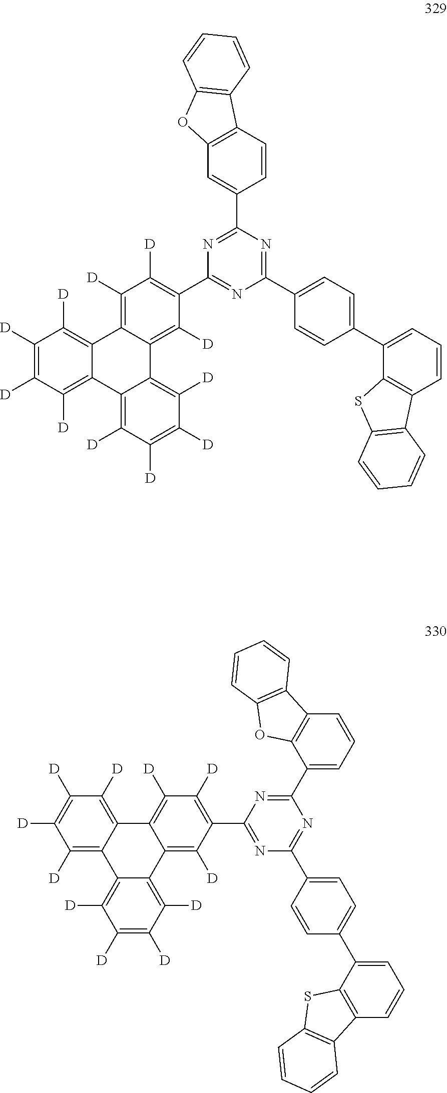 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4oc4ccccc45)n3)c([2H])c([2H])c21]

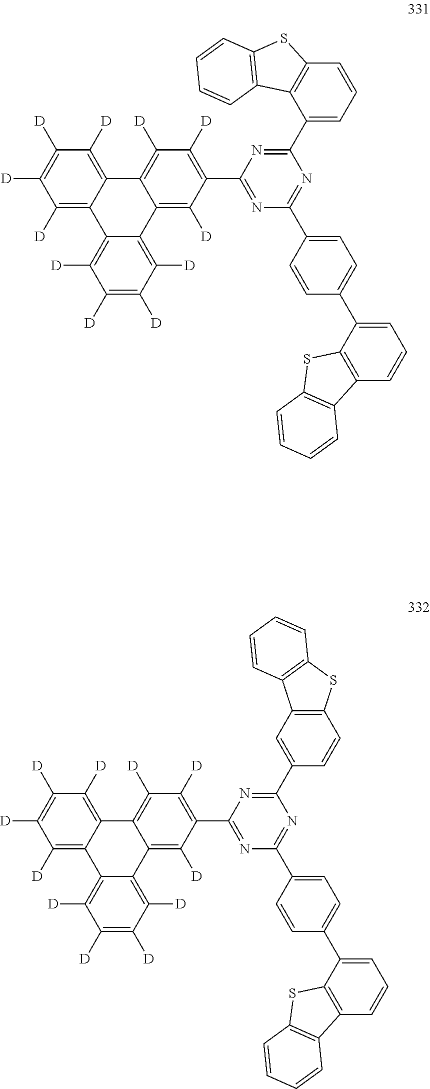 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5sc6ccccc6c5c4)n3)c([2H])c([2H])c21]

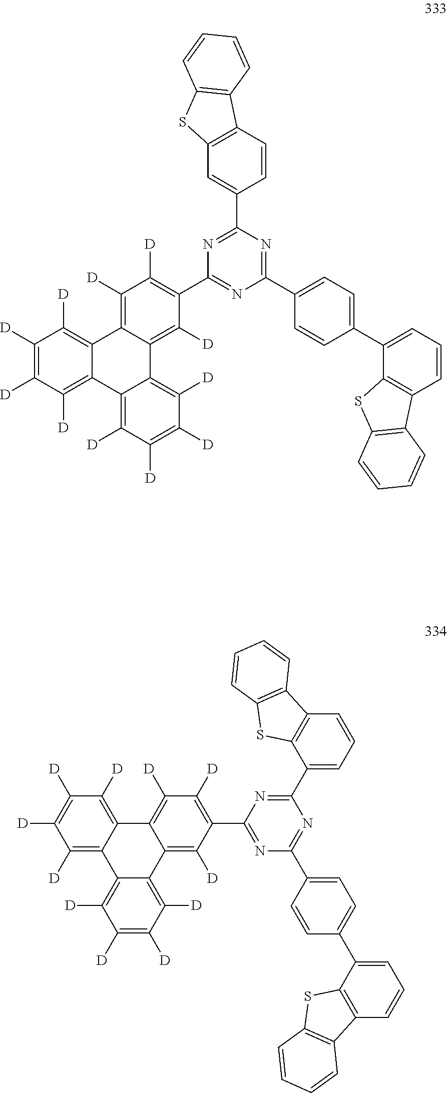 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4sc4ccccc45)n3)c([2H])c([2H])c21]

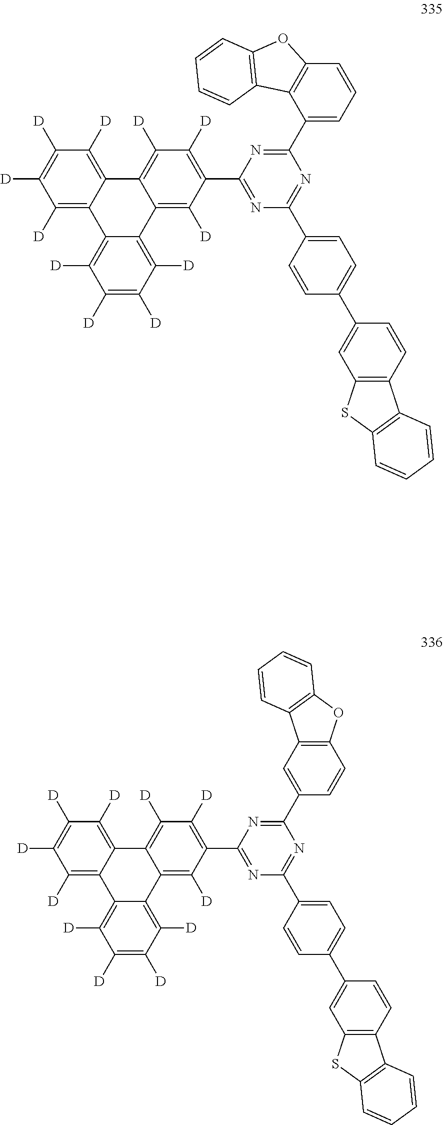 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5oc6ccccc6c5c4)n3)c([2H])c([2H])c21]

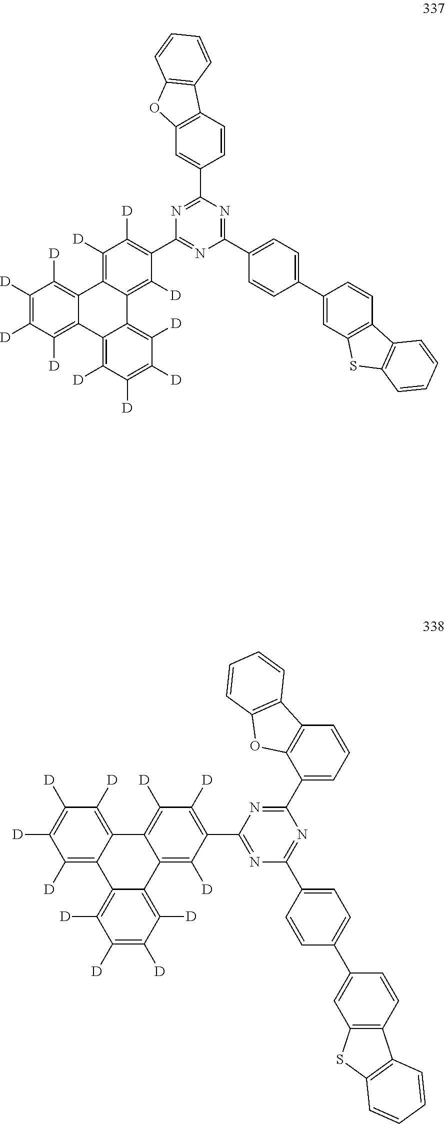 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4oc4ccccc45)n3)c([2H])c([2H])c21]

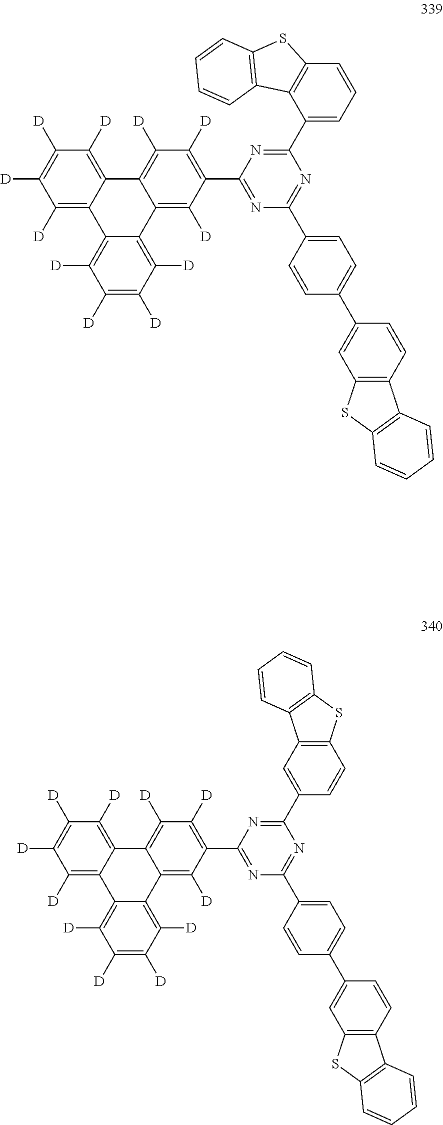 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5sc6ccccc6c5c4)n3)c([2H])c([2H])c21]

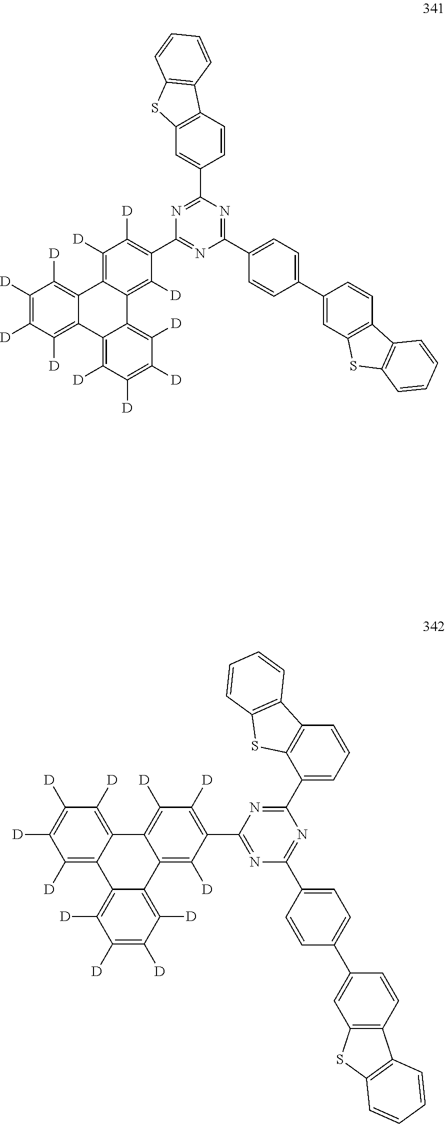 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)sc5ccccc56)cc4)nc(-c4cccc5c4sc4ccccc45)n3)c([2H])c([2H])c21]

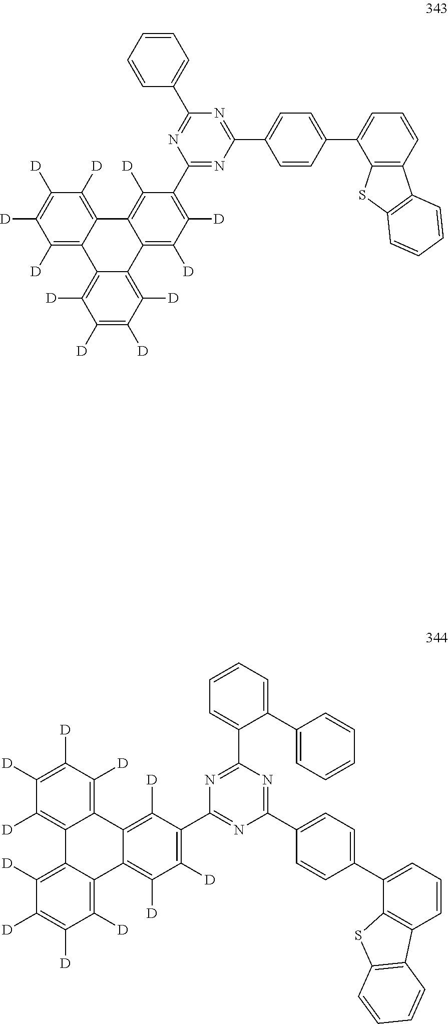 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5sc5ccccc56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccccc4-c4ccccc4)n3)c([2H])c([2H])c21]

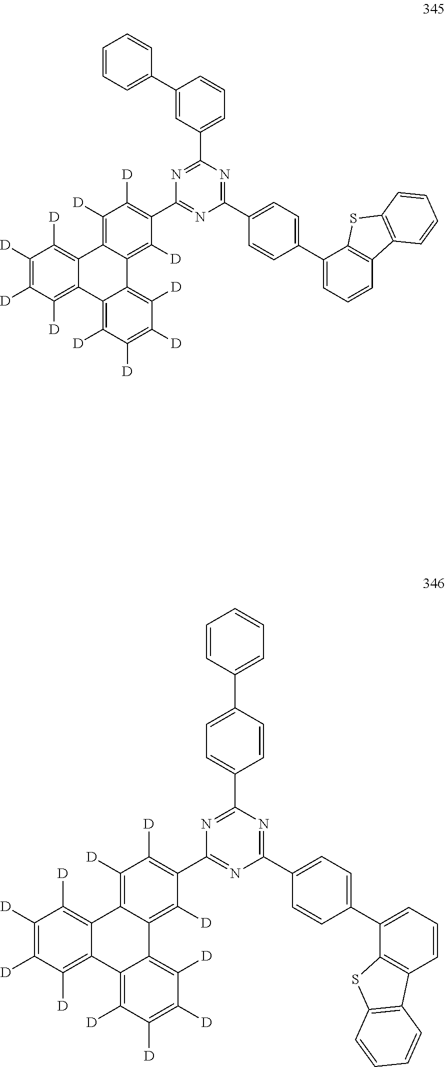 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6c5sc5ccccc56)cc4)n3)c([2H])c([2H])c21]

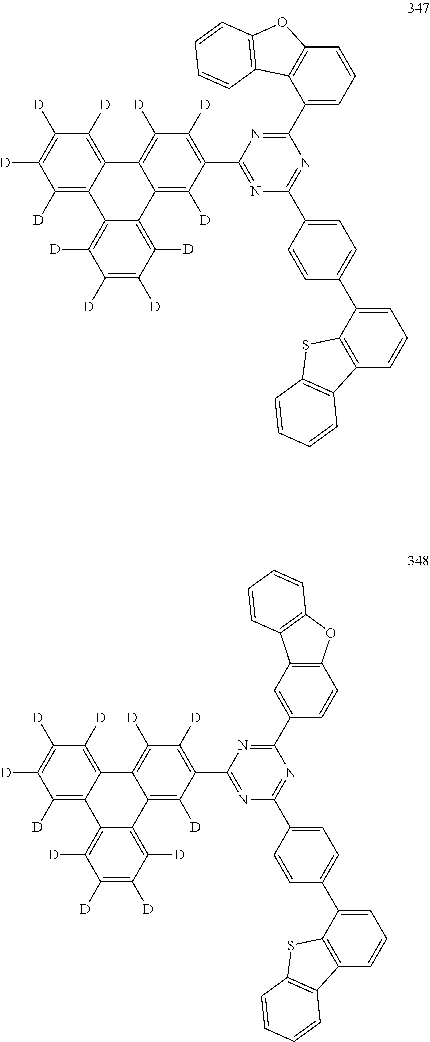 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5oc6ccccc6c5c4)n3)c([2H])c([2H])c21]

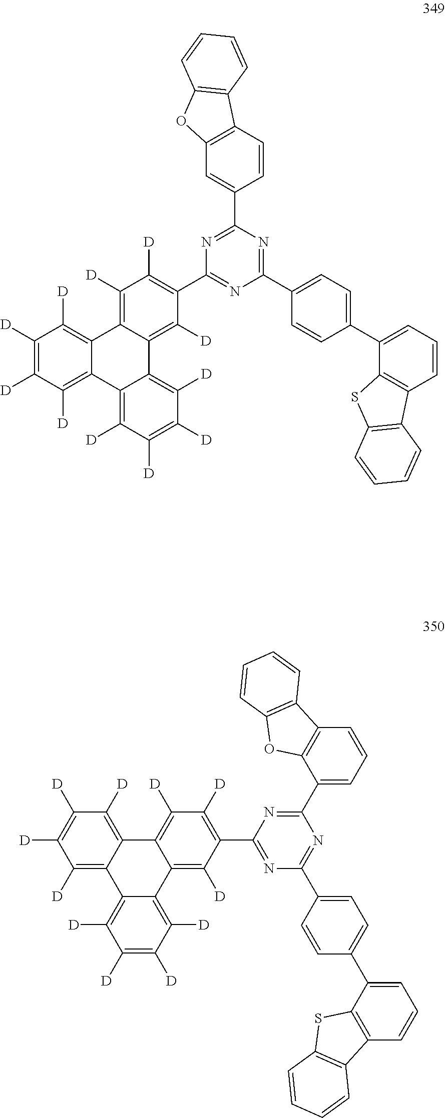 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4oc4ccccc45)n3)c([2H])c([2H])c21]

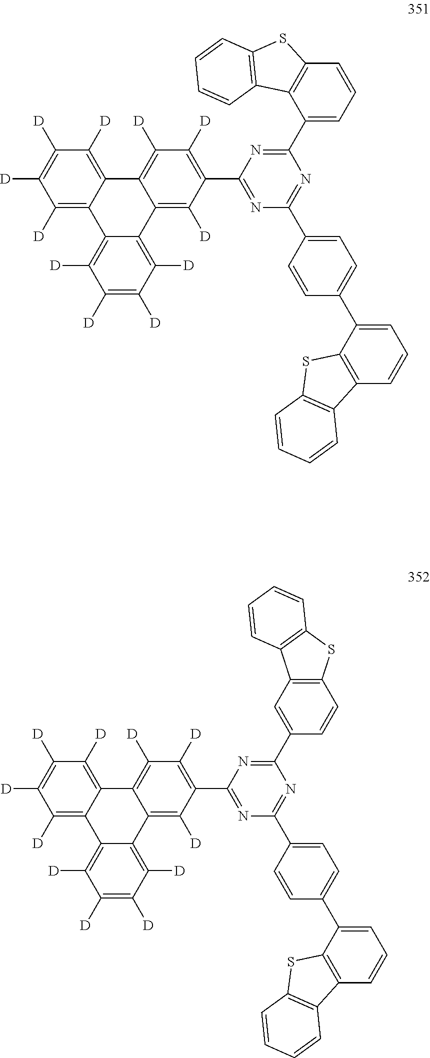 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5sc6ccccc6c5c4)n3)c([2H])c([2H])c21]

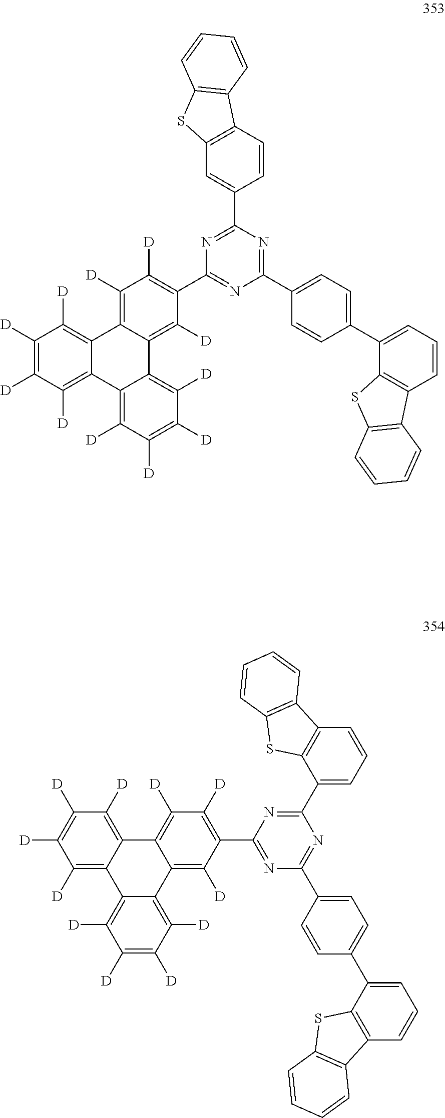 [SMILES: [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5sc5ccccc56)cc4)nc(-c4cccc5c4sc4ccccc45)n3)c([2H])c([2H])c21]

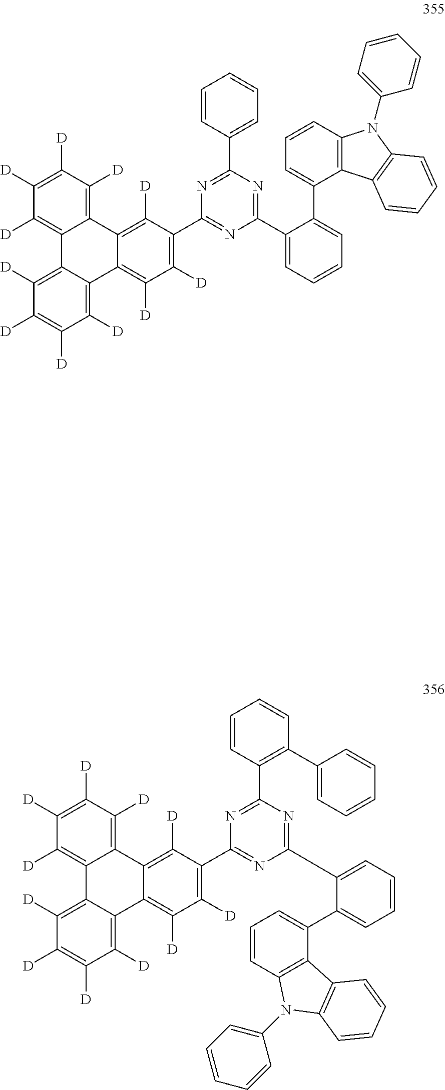 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c4c4ccccc4n5-c4ccccc4)n3)c([2H])c([2H])c21]

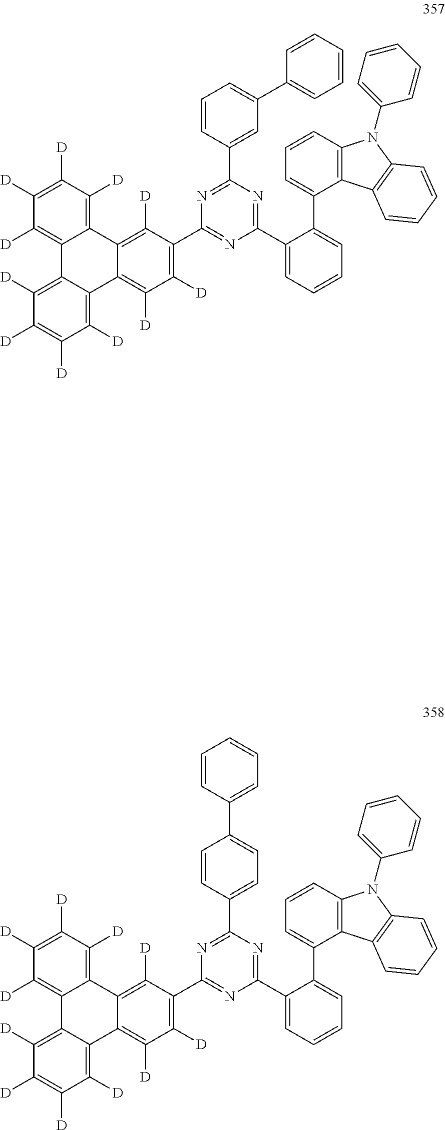 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c4c4ccccc4n5-c4ccccc4)n3)c([2H])c([2H])c21]

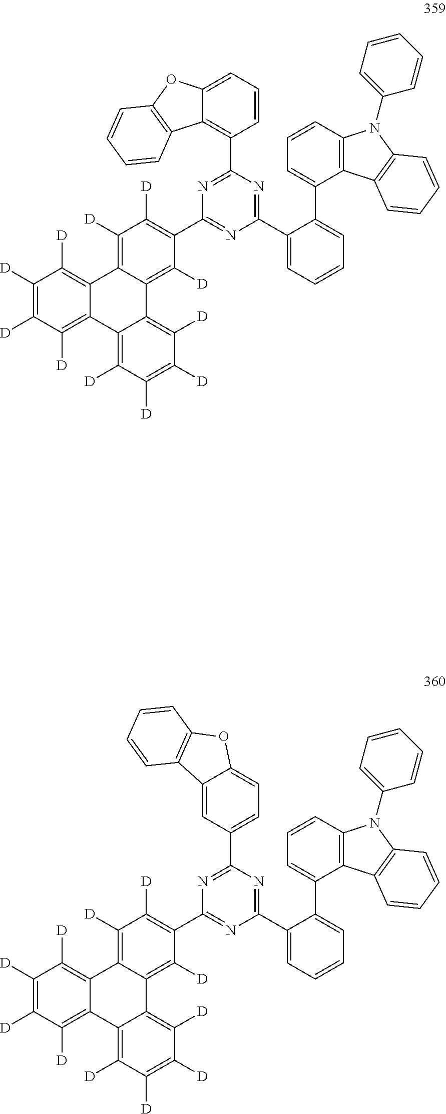 [SMILES: [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5c4c4ccccc4n5-c4ccccc4)n3)c([2H])c([2H])c21]

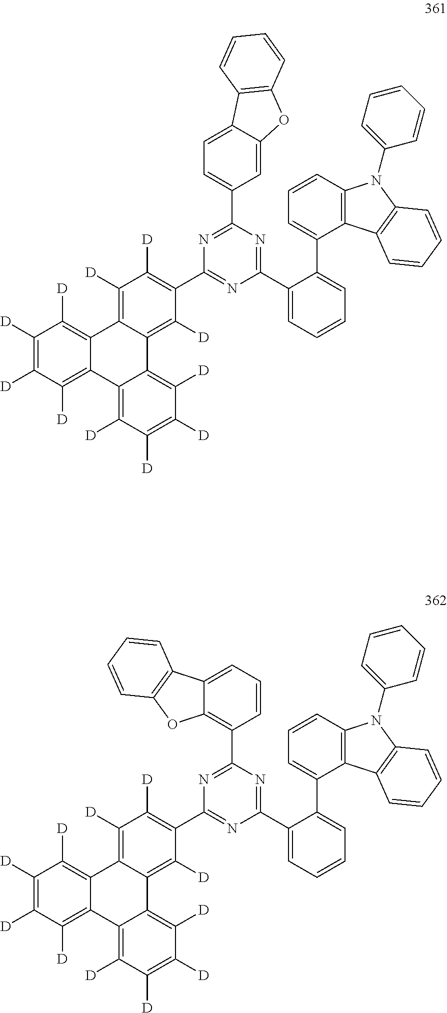 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5c4oc4ccccc45)n3)c([2H])c([2H])c21]

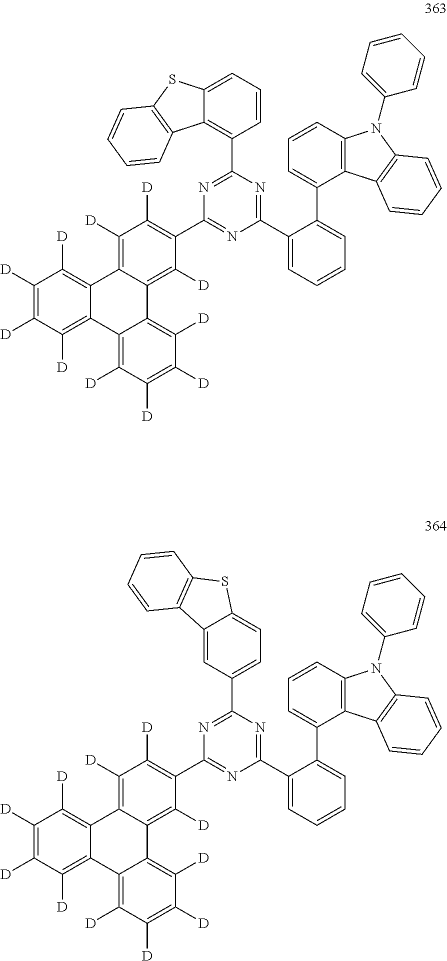 [SMILES: [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5c4c4ccccc4n5-c4ccccc4)n3)c([2H])c([2H])c21]

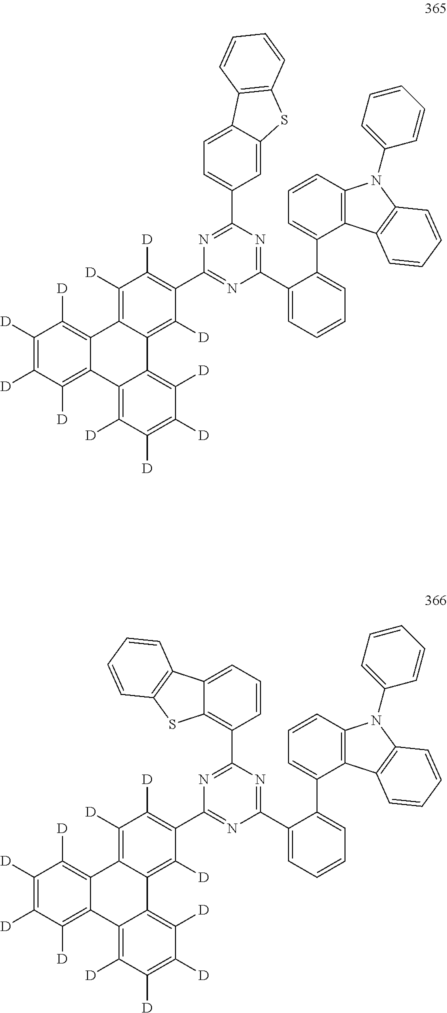 [SMILES: [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5c4c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c4c4ccccc4n5-c4ccccc4)nc(-c4cccc5c4sc4ccccc45)n3)c([2H])c([2H])c21]

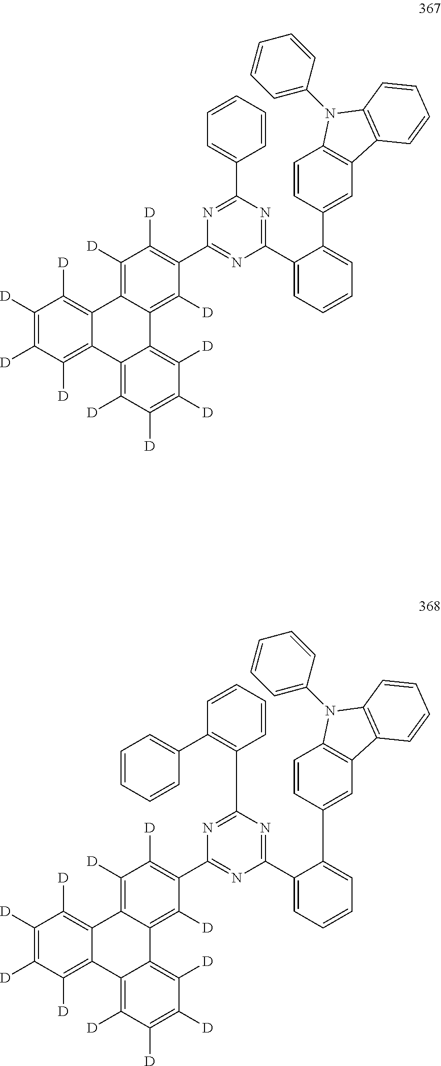 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5c(c4)c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c(c4)c4ccccc4n5-c4ccccc4)n3)c([2H])c([2H])c21]

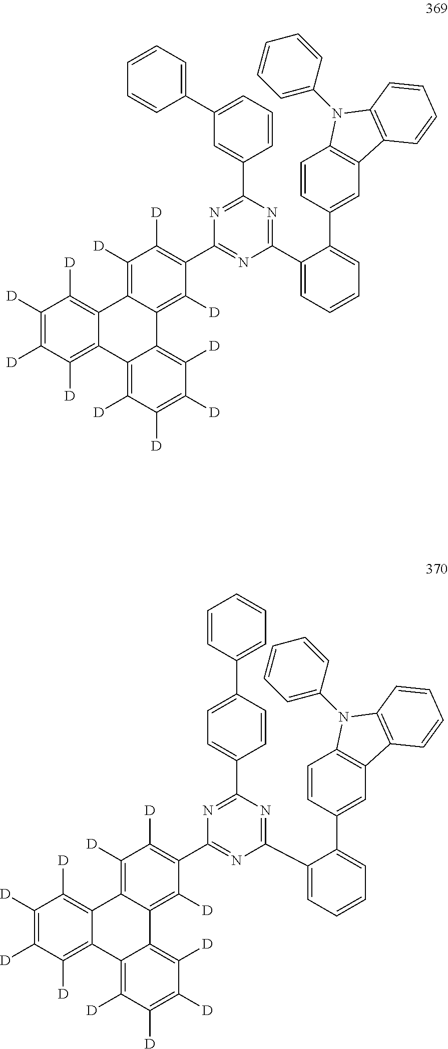 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5c(c4)c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4ccc5c(c4)c4ccccc4n5-c4ccccc4)n3)c([2H])c([2H])c21]

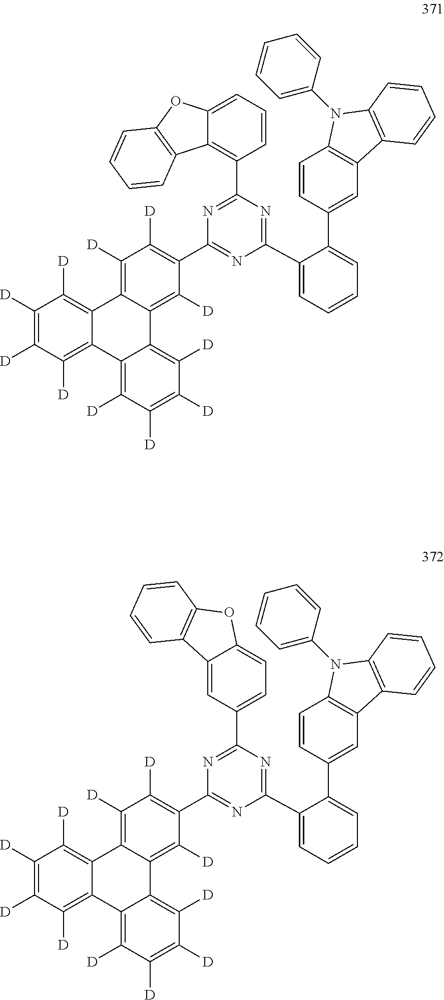 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)c4ccccc4n5-c4ccccc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4ccc5c(c4)c4ccccc4n5-c4ccccc4)n3)c([2H])c([2H])c21]

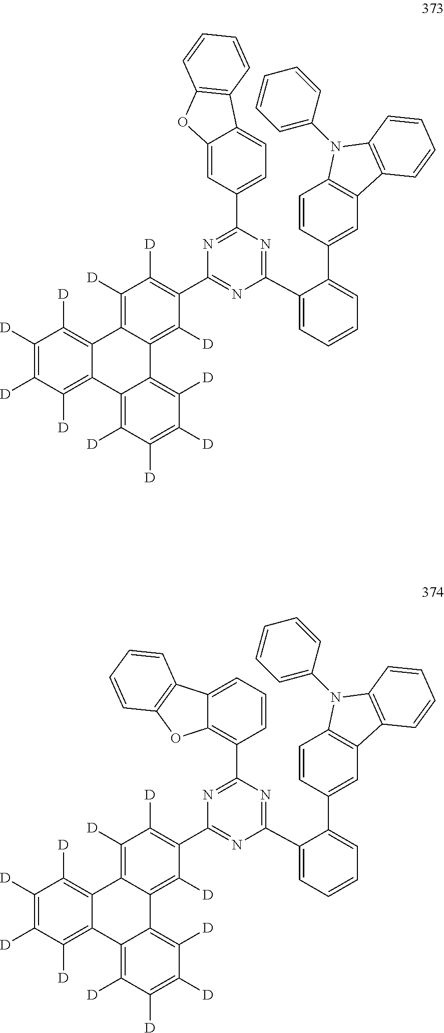 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4ccc5c(c4)c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c(c4)c4ccccc4n5-c4ccccc4)nc(-c4cccc5c4oc4ccccc45)n3)c([2H])c([2H])c21]

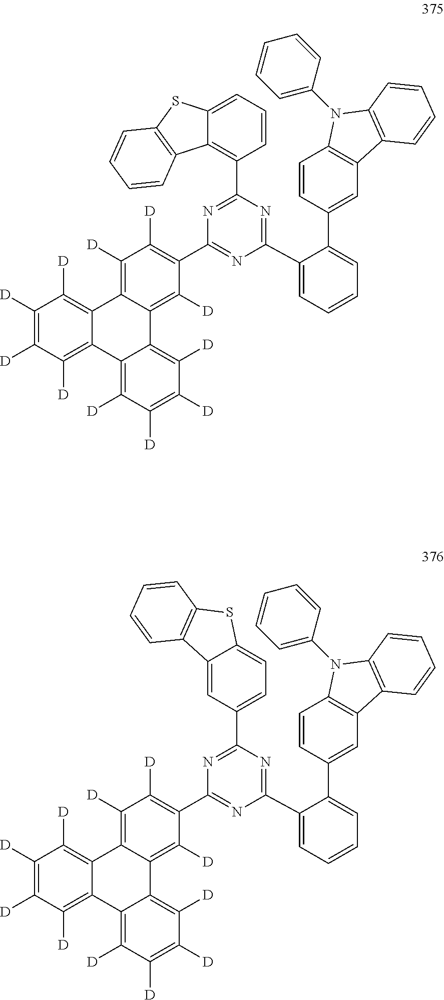 [SMILES: [2H]c1c([2H])c([2H])c2c(c1[2H])c1c([2H])c([2H])c([2H])c([2H])c1c1c([2H])c(-c3nc(-c4ccccc4-c4ccc5c(c4)c4ccccc4n5-c4ccccc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4ccc5c(c4)c4ccccc4n5-c4ccccc4)n3)c([2H])c([2H])c21]

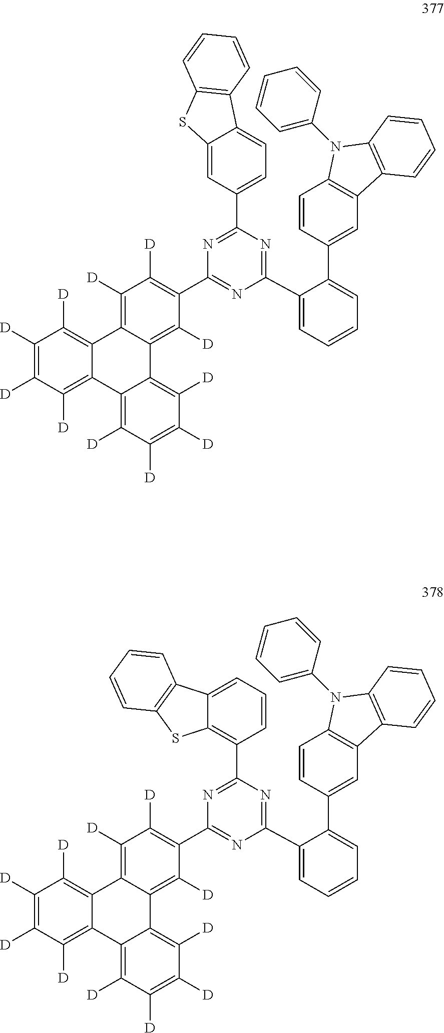 [SMILES: [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4ccc5c(c4)c4ccccc4n5-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c(c4)c4ccccc4n5-c4ccccc4)nc(-c4cccc5c4sc4ccccc45)n3)c([2H])c([2H])c21]

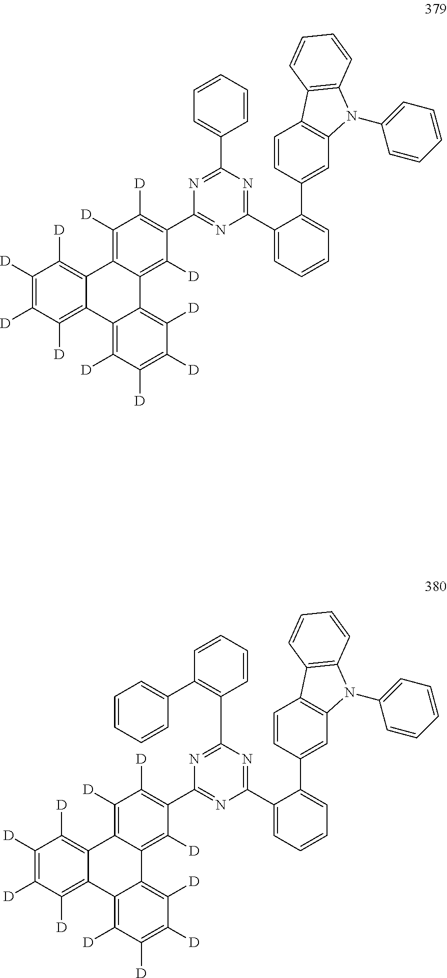 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4ccc5c6ccccc6n(-c6ccccc6)c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4ccc5c6ccccc6n(-c6ccccc6)c5c4)n3)c([2H])c([2H])c21]

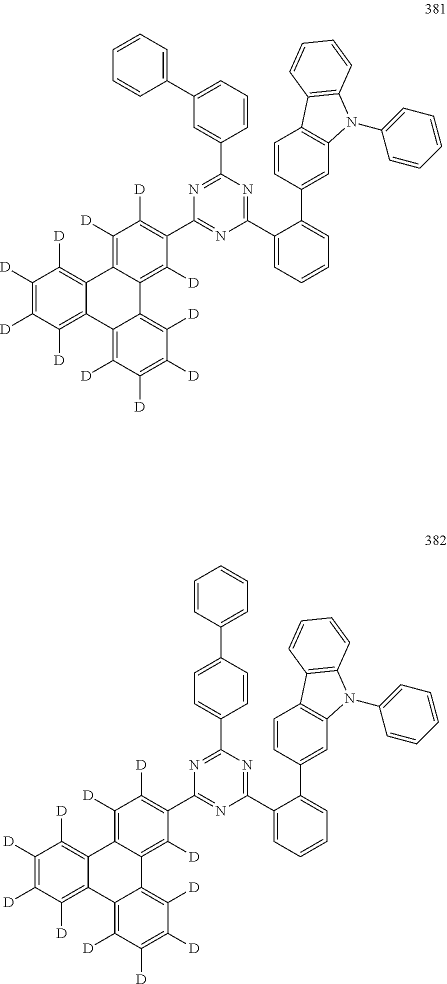 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4ccc5c6ccccc6n(-c6ccccc6)c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4ccc5c6ccccc6n(-c6ccccc6)c5c4)n3)c([2H])c([2H])c21]

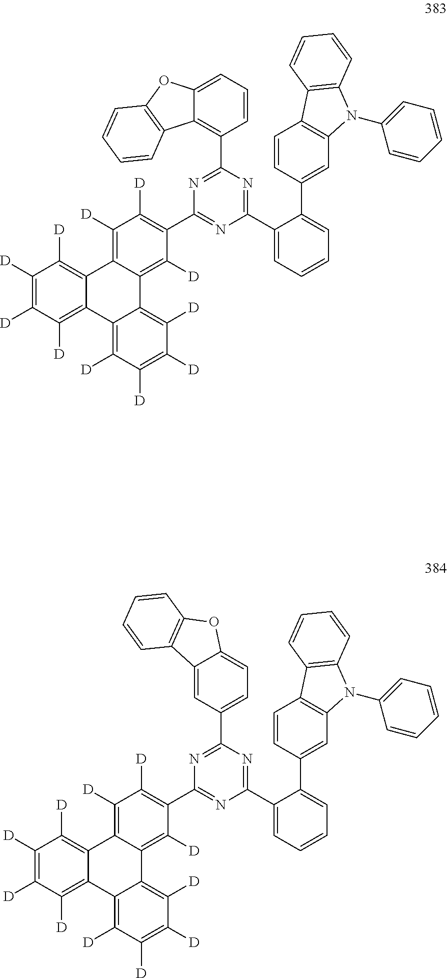 [SMILES: [2H]c1c([2H])c([2H])c2c(c1[2H])c1c([2H])c([2H])c([2H])c([2H])c1c1c([2H])c(-c3nc(-c4ccccc4-c4ccc5c6ccccc6n(-c6ccccc6)c5c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4ccc5c6ccccc6n(-c6ccccc6)c5c4)n3)c([2H])c([2H])c21]

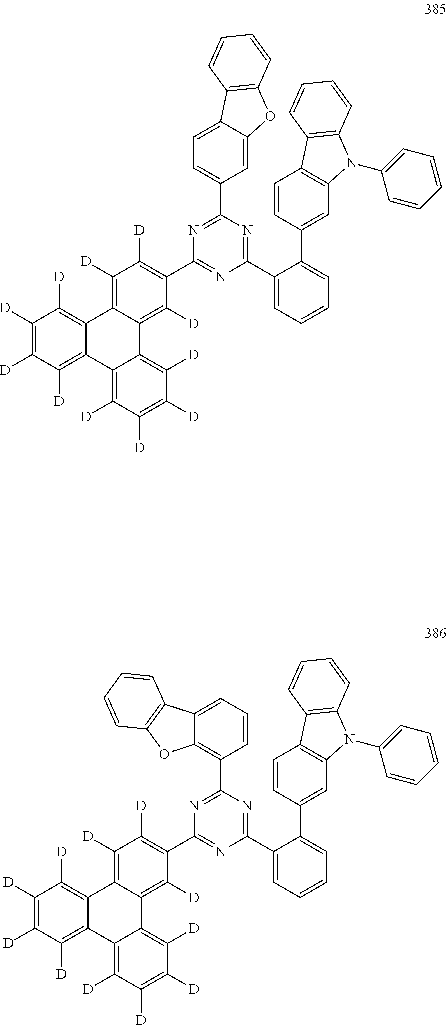 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4ccc5c6ccccc6n(-c6ccccc6)c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c6ccccc6n(-c6ccccc6)c5c4)nc(-c4cccc5c4oc4ccccc45)n3)c([2H])c([2H])c21]

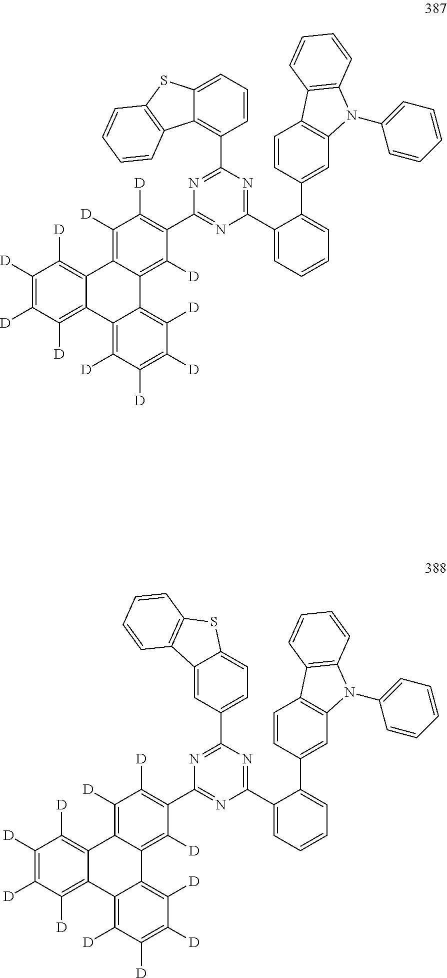 [SMILES: [2H]c1c([2H])c([2H])c2c(c1[2H])c1c([2H])c([2H])c([2H])c([2H])c1c1c([2H])c(-c3nc(-c4ccccc4-c4ccc5c6ccccc6n(-c6ccccc6)c5c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4ccc5c6ccccc6n(-c6ccccc6)c5c4)n3)c([2H])c([2H])c21]

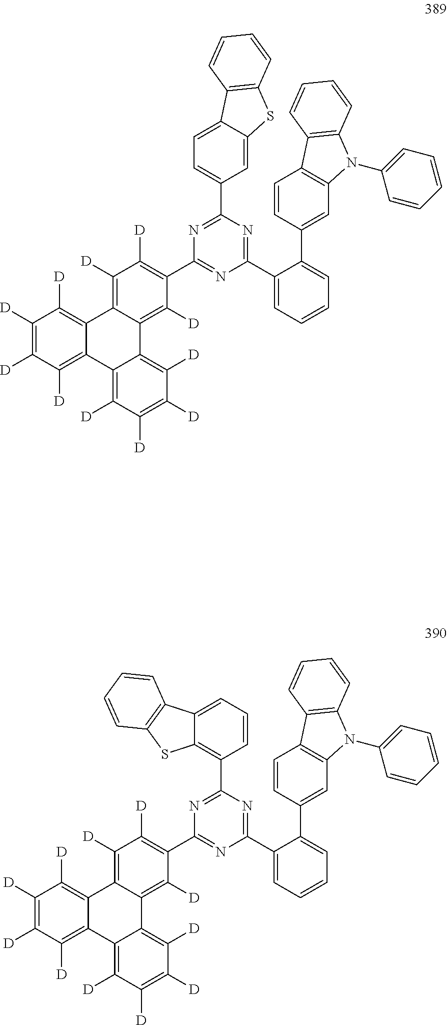 [SMILES: [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4ccc5c6ccccc6n(-c6ccccc6)c5c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccc5c6ccccc6n(-c6ccccc6)c5c4)nc(-c4cccc5c4sc4ccccc45)n3)c([2H])c([2H])c21]

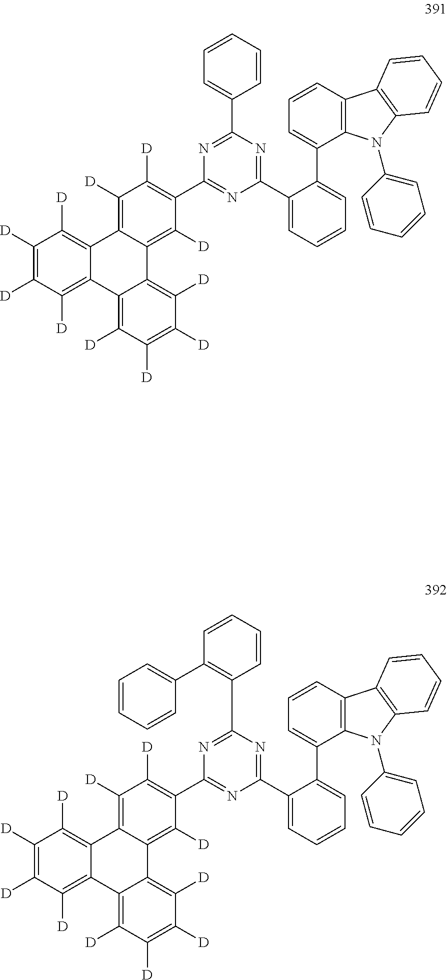 [SMILES: [2H]c1c([2H])c([2H])c2c(c1[2H])c1c([2H])c([2H])c([2H])c([2H])c1c1c([2H])c(-c3nc(-c4ccccc4)nc(-c4ccccc4-c4cccc5c6ccccc6n(-c6ccccc6)c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4ccccc4)nc(-c4ccccc4-c4cccc5c6ccccc6n(-c6ccccc6)c45)n3)c([2H])c([2H])c21]

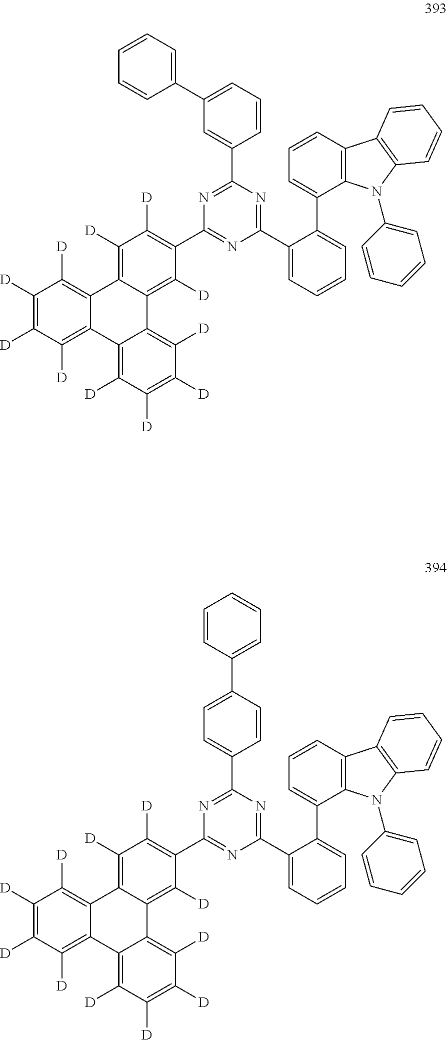 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4ccccc4-c4cccc5c6ccccc6n(-c6ccccc6)c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccccc4-c4cccc5c6ccccc6n(-c6ccccc6)c45)n3)c([2H])c([2H])c21]

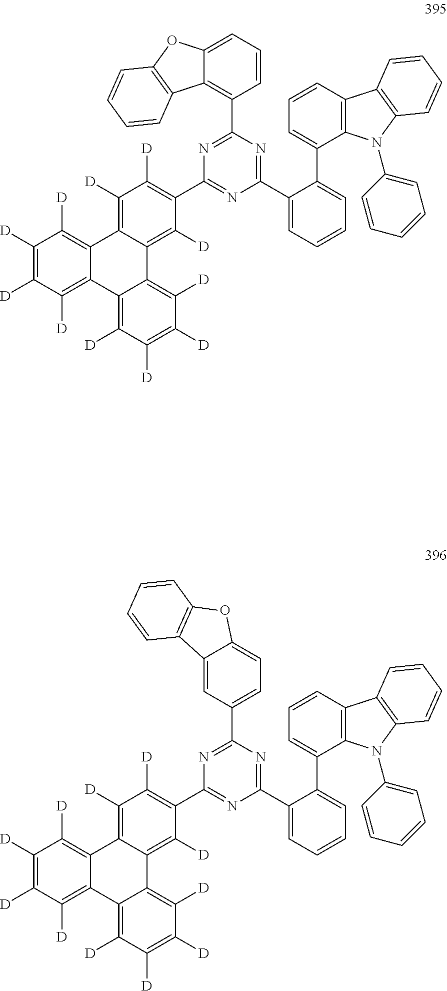 [SMILES: [2H]c1c([2H])c([2H])c2c(c1[2H])c1c([2H])c([2H])c([2H])c([2H])c1c1c([2H])c(-c3nc(-c4ccccc4-c4cccc5c6ccccc6n(-c6ccccc6)c45)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5oc6ccccc6c5c4)nc(-c4ccccc4-c4cccc5c6ccccc6n(-c6ccccc6)c45)n3)c([2H])c([2H])c21]

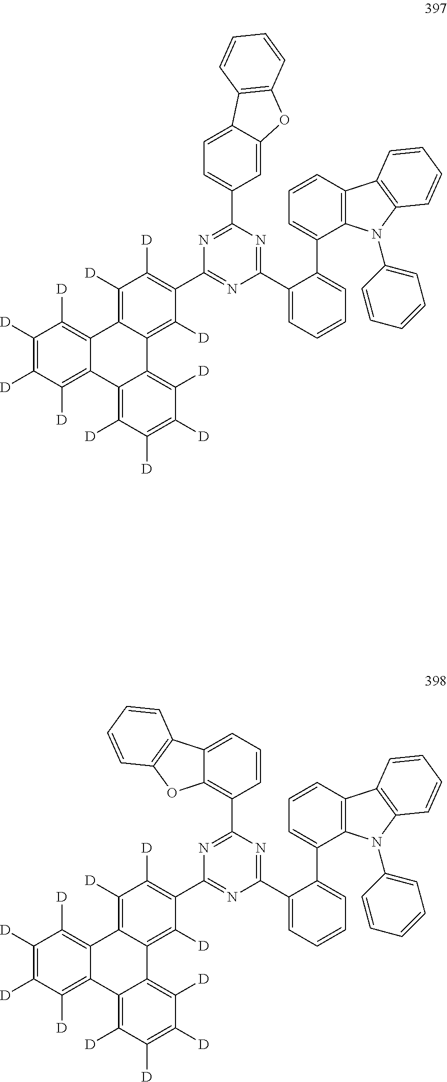 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccccc4-c4cccc5c6ccccc6n(-c6ccccc6)c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c6ccccc6n(-c6ccccc6)c45)nc(-c4cccc5c4oc4ccccc45)n3)c([2H])c([2H])c21]

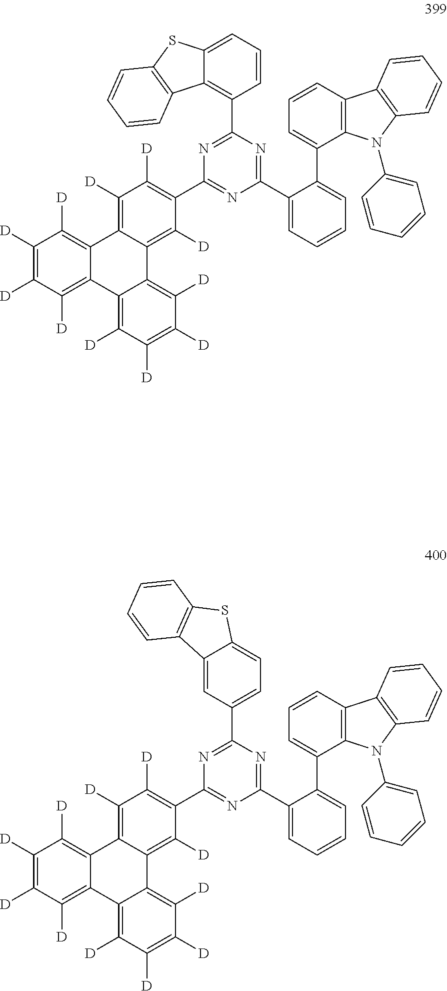 [SMILES: [2H]c1c([2H])c([2H])c2c(c1[2H])c1c([2H])c([2H])c([2H])c([2H])c1c1c([2H])c(-c3nc(-c4ccccc4-c4cccc5c6ccccc6n(-c6ccccc6)c45)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc5sc6ccccc6c5c4)nc(-c4ccccc4-c4cccc5c6ccccc6n(-c6ccccc6)c45)n3)c([2H])c([2H])c21]

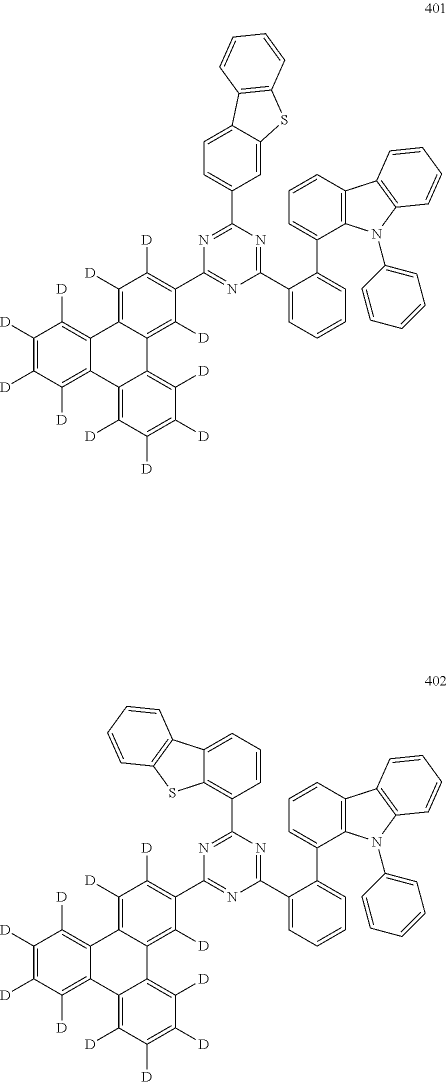 [SMILES: [2H]c1c([2H])c([2H])c2c(c1[2H])c1c([2H])c([2H])c([2H])c([2H])c1c1c([2H])c(-c3nc(-c4ccc5c(c4)sc4ccccc45)nc(-c4ccccc4-c4cccc5c6ccccc6n(-c6ccccc6)c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccccc4-c4cccc5c6ccccc6n(-c6ccccc6)c45)nc(-c4cccc5c4sc4ccccc45)n3)c([2H])c([2H])c21]

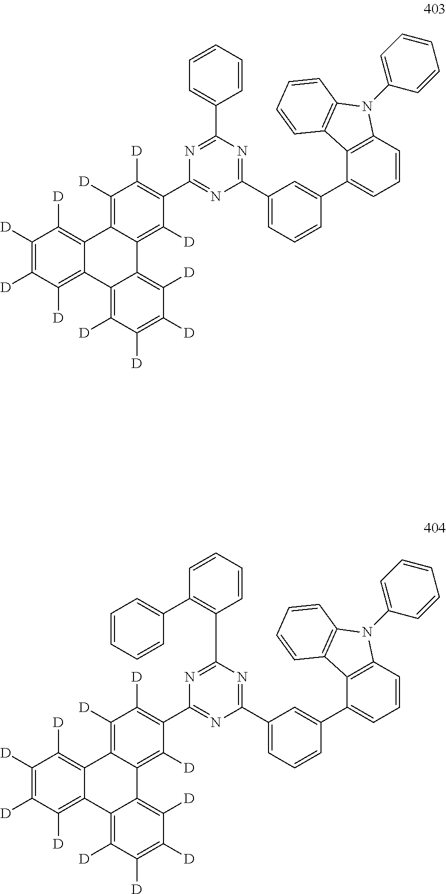 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c5c5ccccc5n6-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4ccccc4-c4ccccc4)n3)c([2H])c([2H])c21]

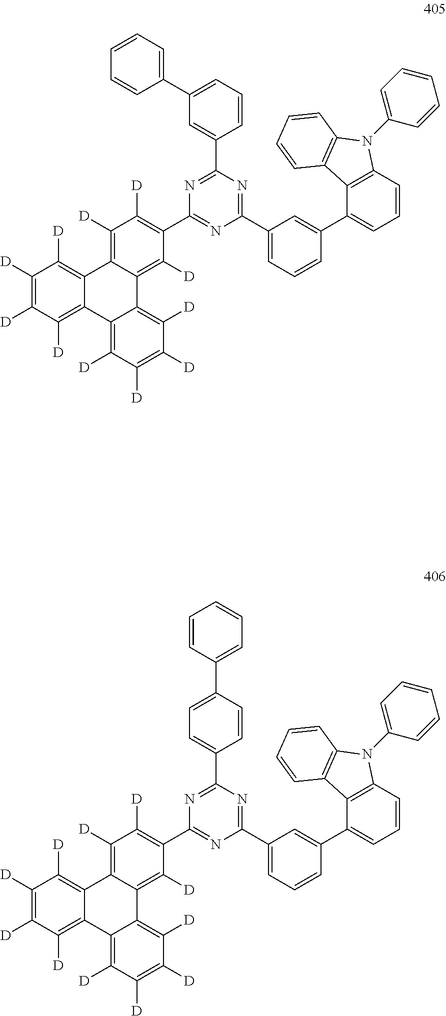 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c5c5ccccc5n6-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5cccc6c5c5ccccc5n6-c5ccccc5)c4)n3)c([2H])c([2H])c21]

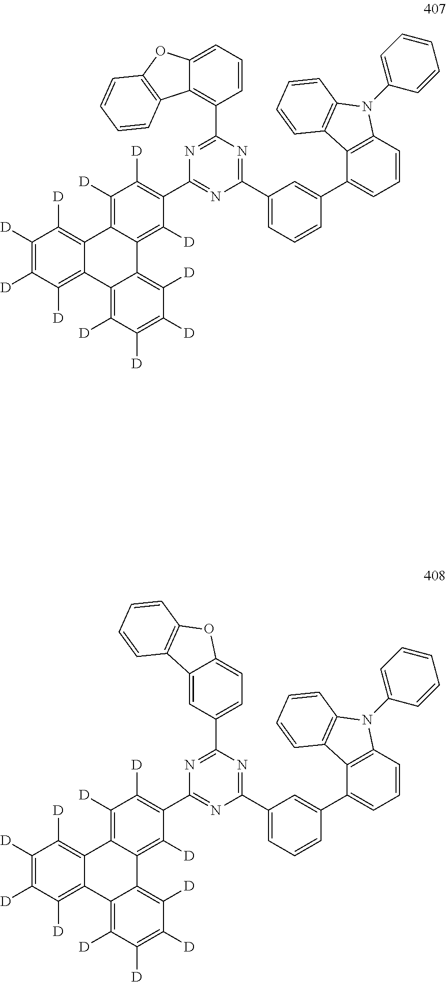 [SMILES: [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4ccc5oc6ccccc6c5c4)n3)c([2H])c([2H])c21]

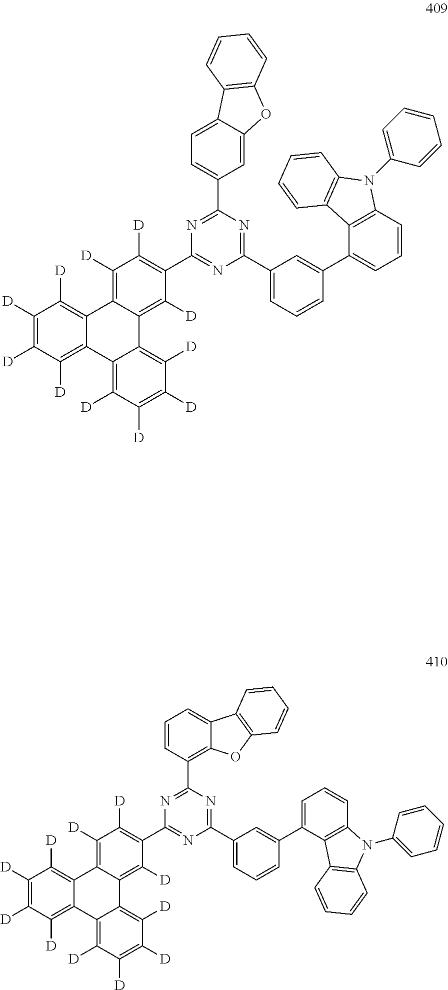 [SMILES: [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4cccc5c4oc4ccccc45)n3)c([2H])c([2H])c21]

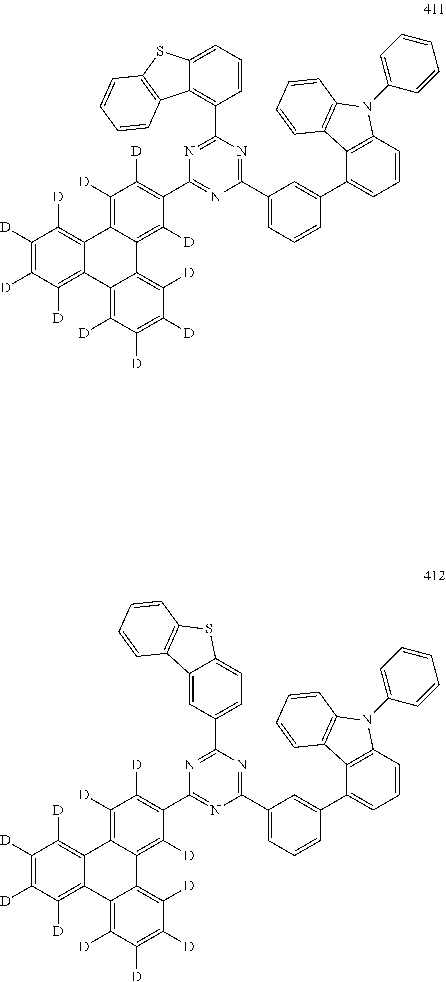 [SMILES: [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4ccc5sc6ccccc6c5c4)n3)c([2H])c([2H])c21]

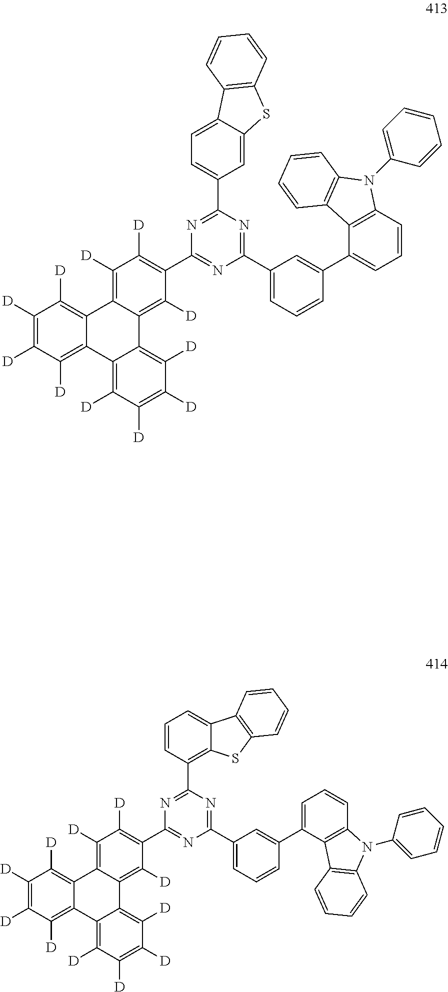 [SMILES: [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c5c5ccccc5n6-c5ccccc5)c4)nc(-c4cccc5c4sc4ccccc45)n3)c([2H])c([2H])c21]

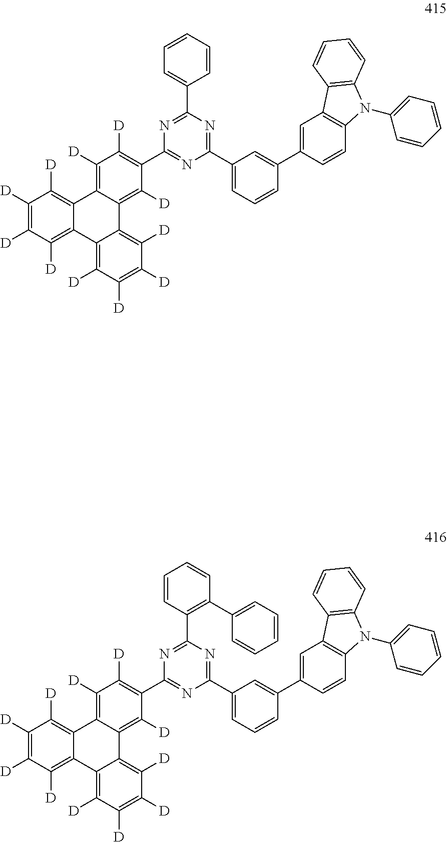 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6c(c5)c5ccccc5n6-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4ccccc4-c4ccccc4)n3)c([2H])c([2H])c21]

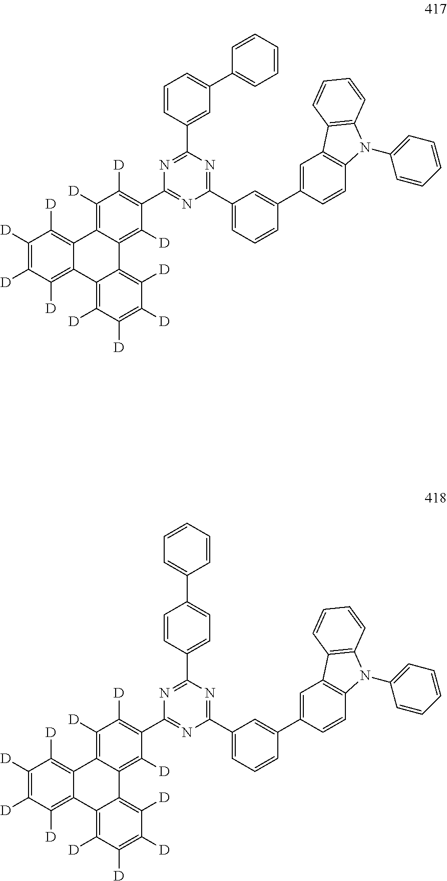 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6c(c5)c5ccccc5n6-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6c(c5)c5ccccc5n6-c5ccccc5)c4)n3)c([2H])c([2H])c21]

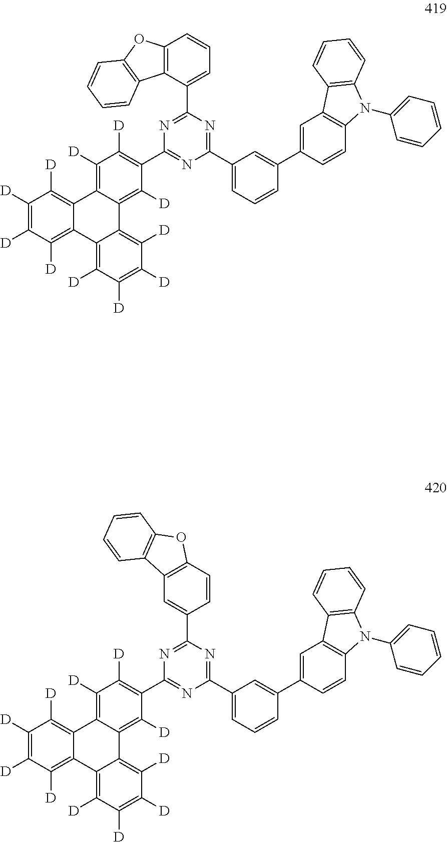 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4ccc5oc6ccccc6c5c4)n3)c([2H])c([2H])c21]

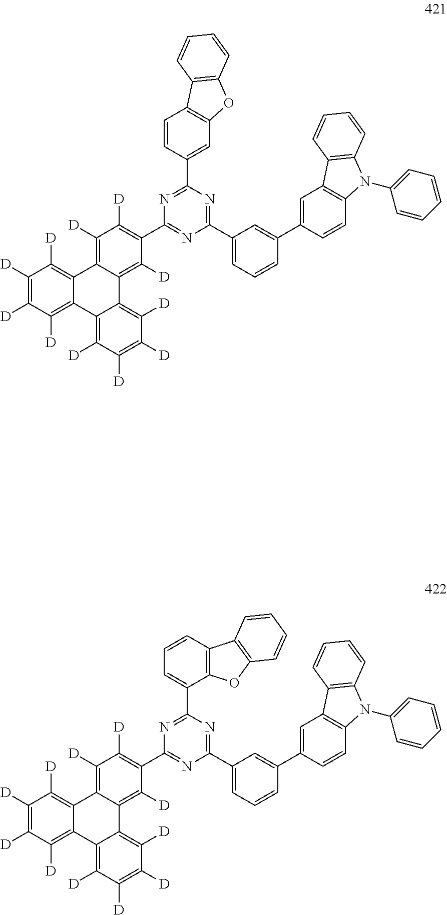 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4cccc5c4oc4ccccc45)n3)c([2H])c([2H])c21]

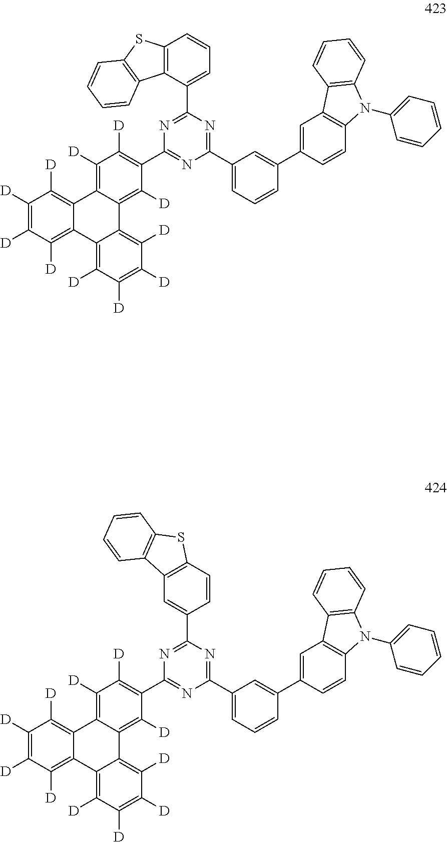 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4ccc5sc6ccccc6c5c4)n3)c([2H])c([2H])c21]

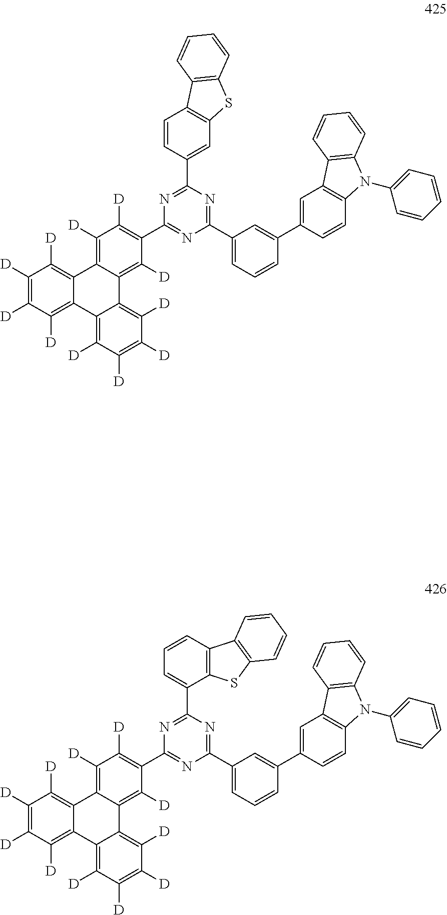 [SMILES: [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c(c5)c5ccccc5n6-c5ccccc5)c4)nc(-c4cccc5c4sc4ccccc45)n3)c([2H])c([2H])c21]

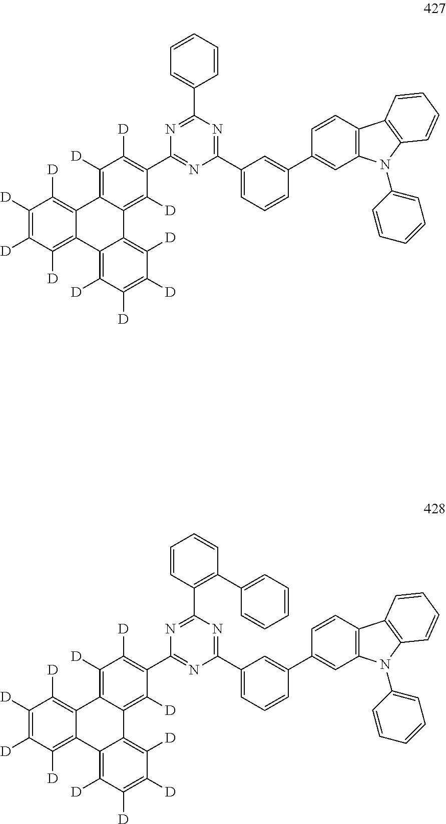 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5ccc6c7ccccc7n(-c7ccccc7)c6c5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4ccccc4-c4ccccc4)n3)c([2H])c([2H])c21]

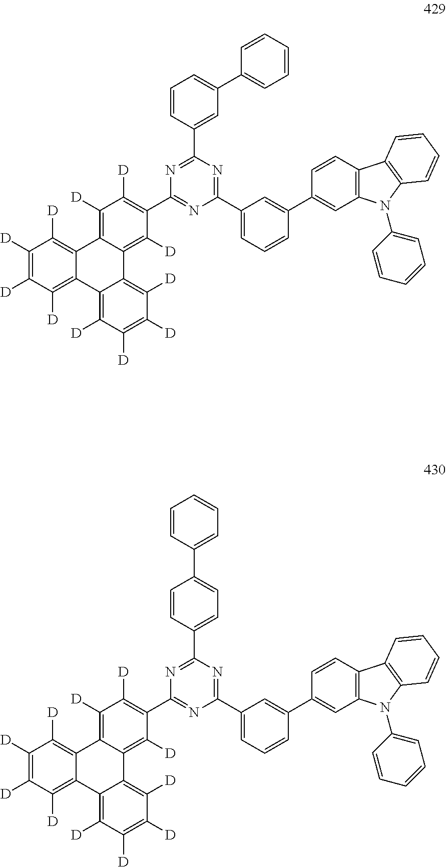 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5ccc6c7ccccc7n(-c7ccccc7)c6c5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5ccc6c7ccccc7n(-c7ccccc7)c6c5)c4)n3)c([2H])c([2H])c21]

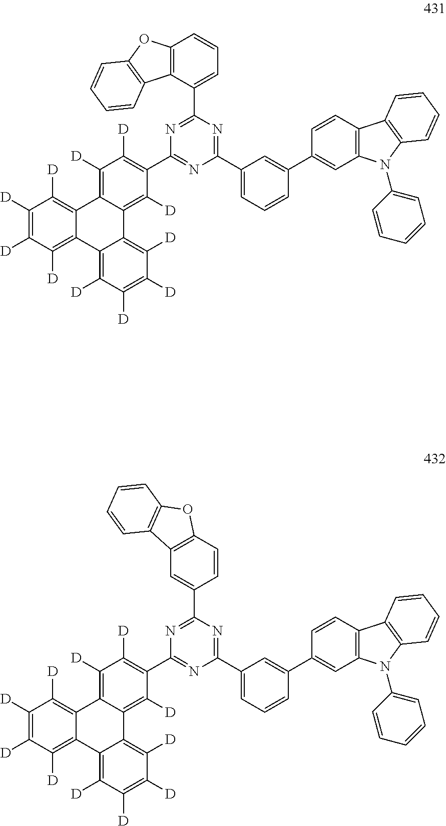 [SMILES: [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4ccc5oc6ccccc6c5c4)n3)c([2H])c([2H])c21]

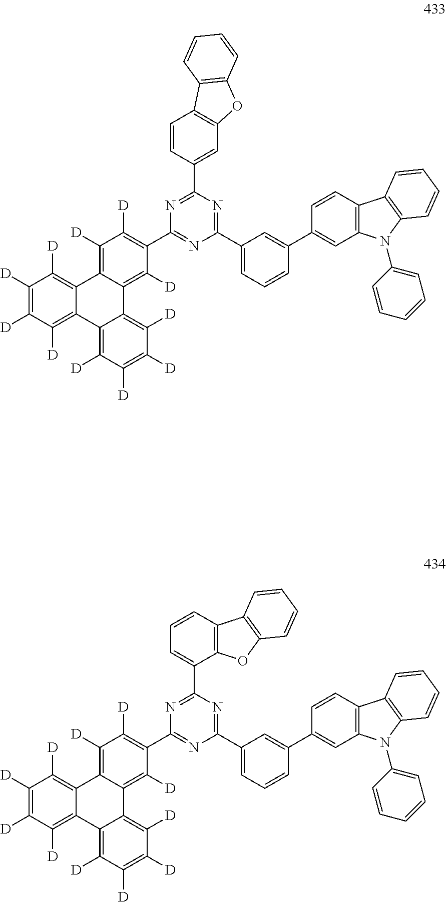 [SMILES: [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4cccc5c4oc4ccccc45)n3)c([2H])c([2H])c21]

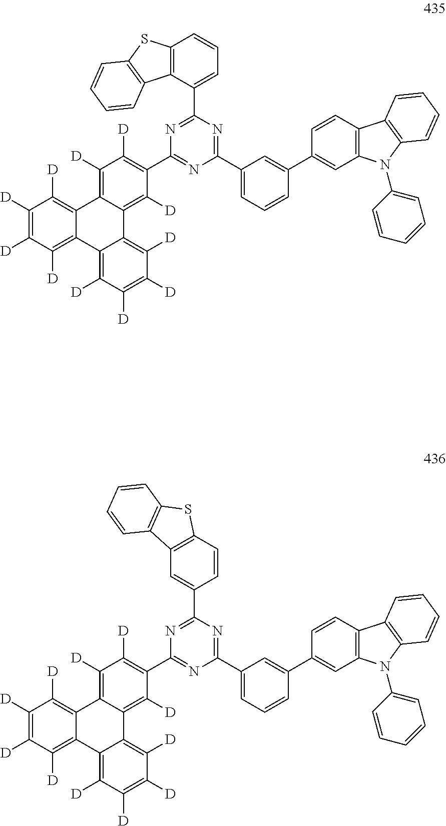 [SMILES: [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4ccc5sc6ccccc6c5c4)n3)c([2H])c([2H])c21]

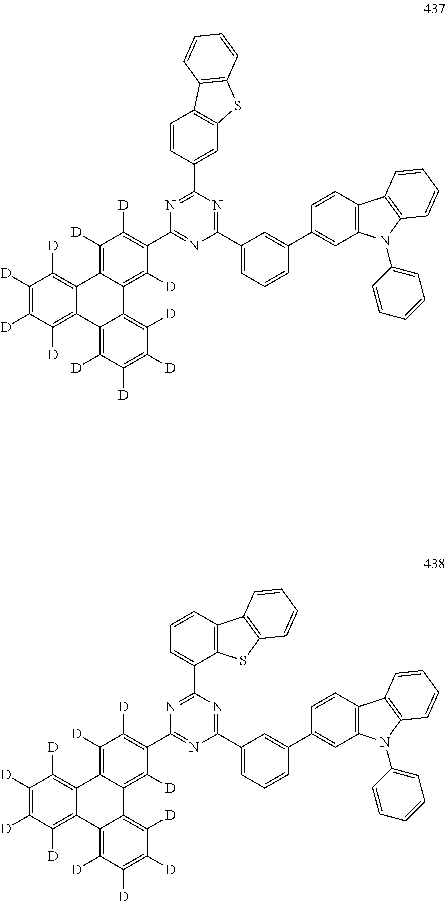 [SMILES: [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5ccc6c7ccccc7n(-c7ccccc7)c6c5)c4)nc(-c4cccc5c4sc4ccccc45)n3)c([2H])c([2H])c21]

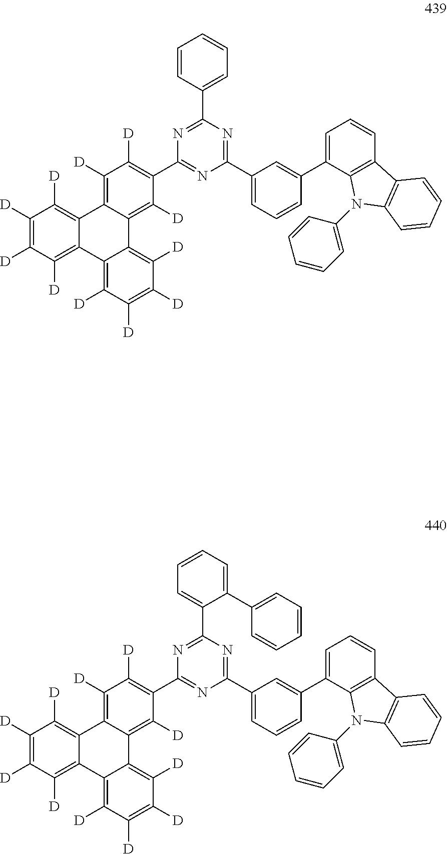 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc(-c5cccc6c7ccccc7n(-c7ccccc7)c56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4ccccc4-c4ccccc4)n3)c([2H])c([2H])c21]

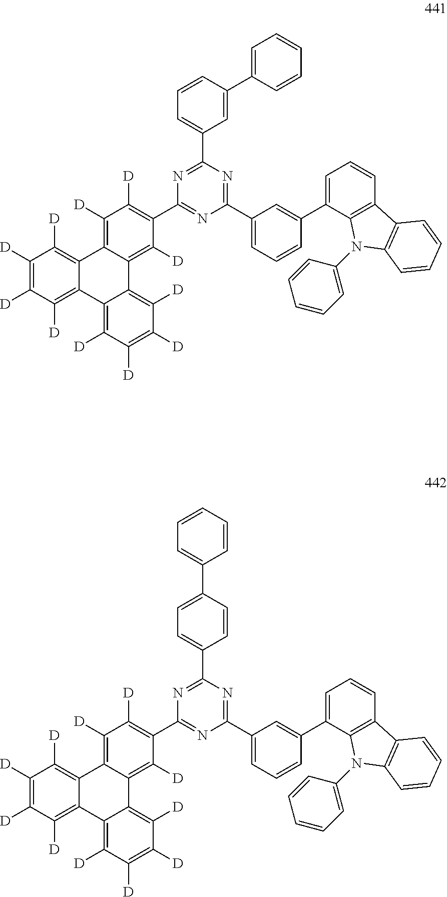 [SMILES: [2H]c1c([2H])c([2H])c2c(c1[2H])c1c([2H])c([2H])c([2H])c([2H])c1c1c([2H])c(-c3nc(-c4cccc(-c5ccccc5)c4)nc(-c4cccc(-c5cccc6c7ccccc7n(-c7ccccc7)c56)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4cccc(-c5cccc6c7ccccc7n(-c7ccccc7)c56)c4)n3)c([2H])c([2H])c21]

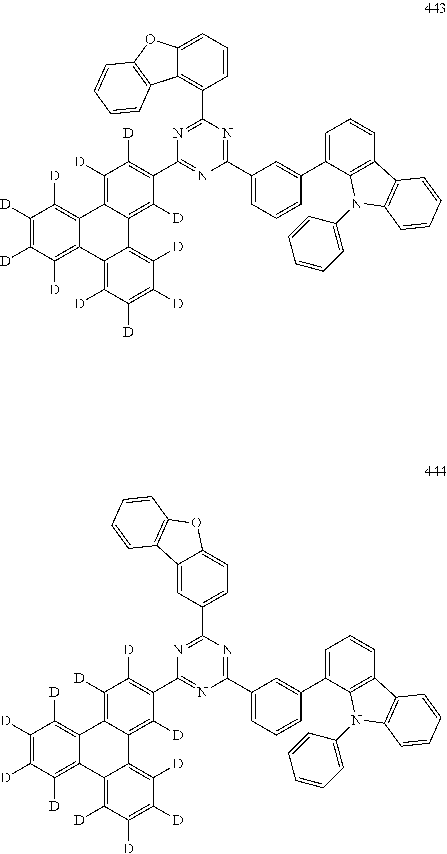 [SMILES: [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4ccc5oc6ccccc6c5c4)n3)c([2H])c([2H])c21]

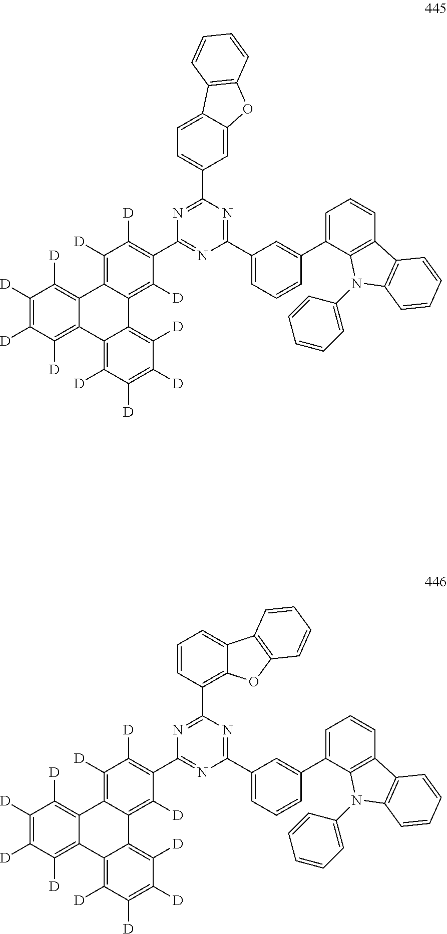 [SMILES: [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4cccc5c4oc4ccccc45)n3)c([2H])c([2H])c21]

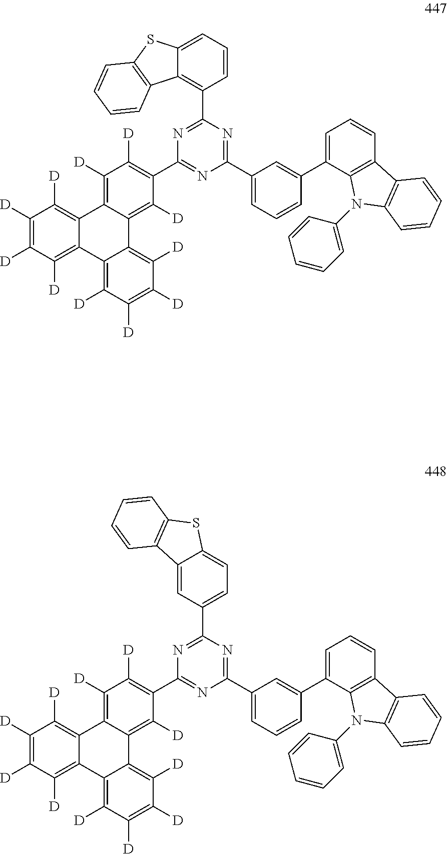 [SMILES: [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4ccc5sc6ccccc6c5c4)n3)c([2H])c([2H])c21]

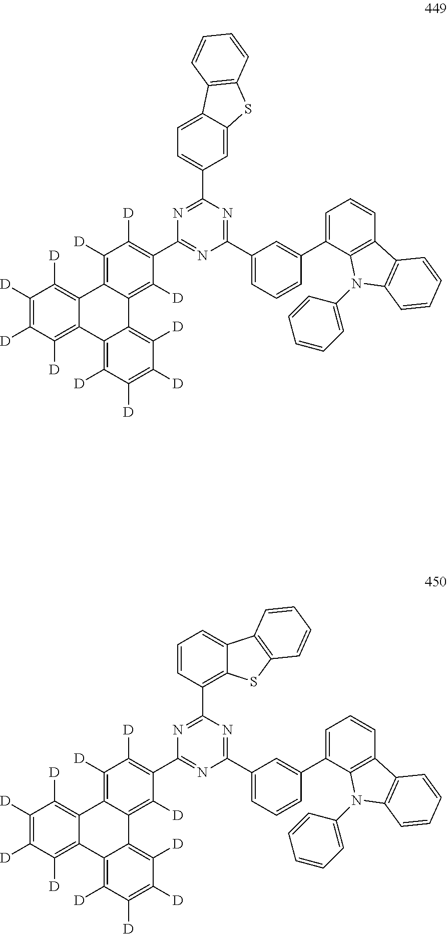 [SMILES: [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4cccc(-c5cccc6c7ccccc7n(-c7ccccc7)c56)c4)nc(-c4cccc5c4sc4ccccc45)n3)c([2H])c([2H])c21]

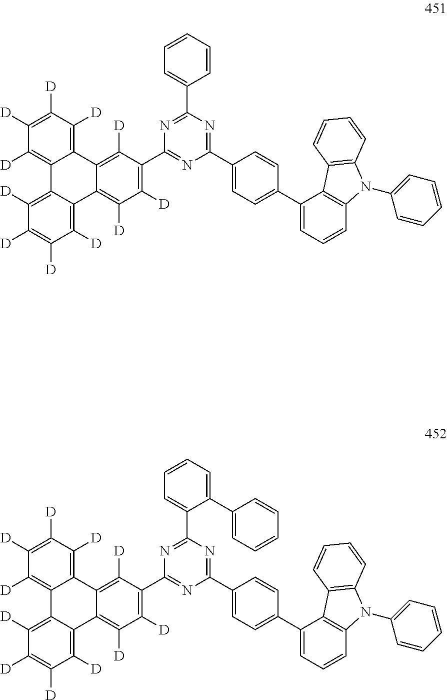 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c5c5ccccc5n6-c5ccccc5)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4ccccc4-c4ccccc4)n3)c([2H])c([2H])c21]

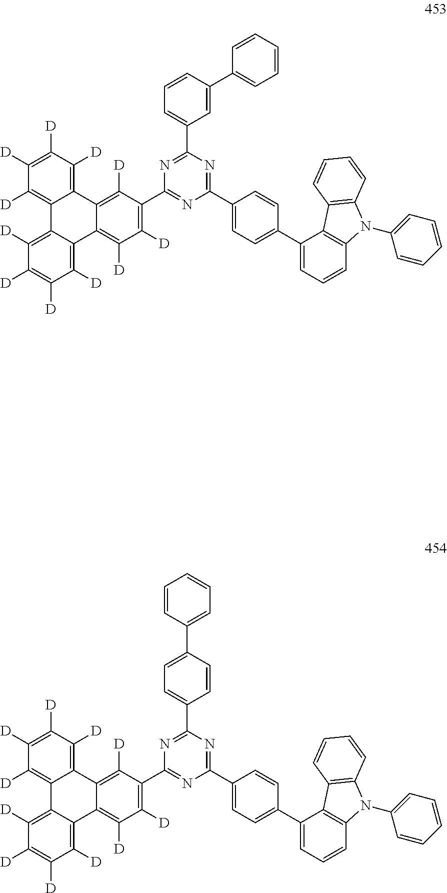 [SMILES: [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5cccc6c5c5ccccc5n6-c5ccccc5)cc4)n3)c([2H])c([2H])c21]

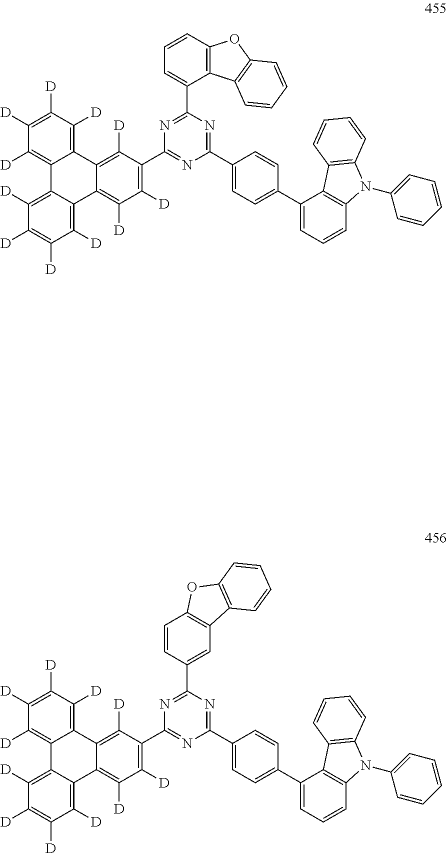 [SMILES: [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4ccc5oc6ccccc6c5c4)n3)c([2H])c([2H])c21]

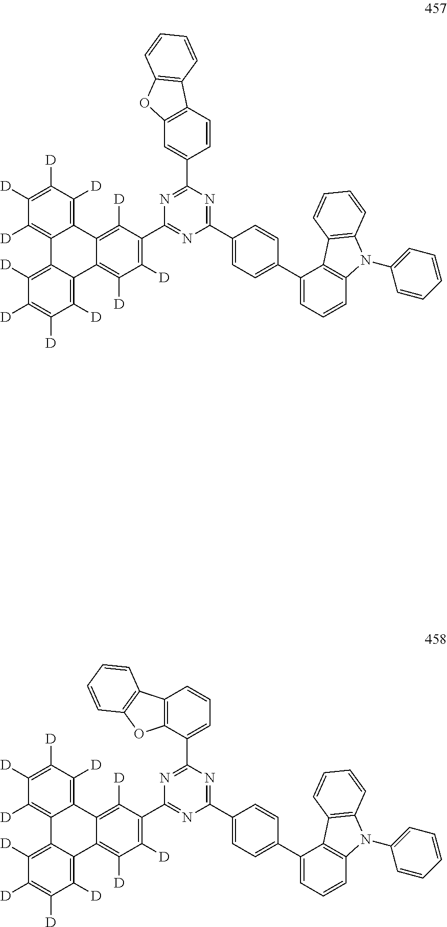 [SMILES: [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4cccc5c4oc4ccccc45)n3)c([2H])c([2H])c21]

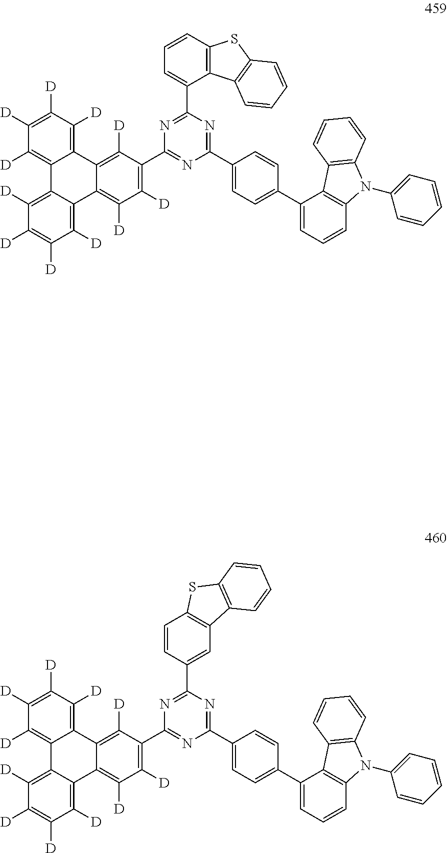 [SMILES: [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4ccc5sc6ccccc6c5c4)n3)c([2H])c([2H])c21]

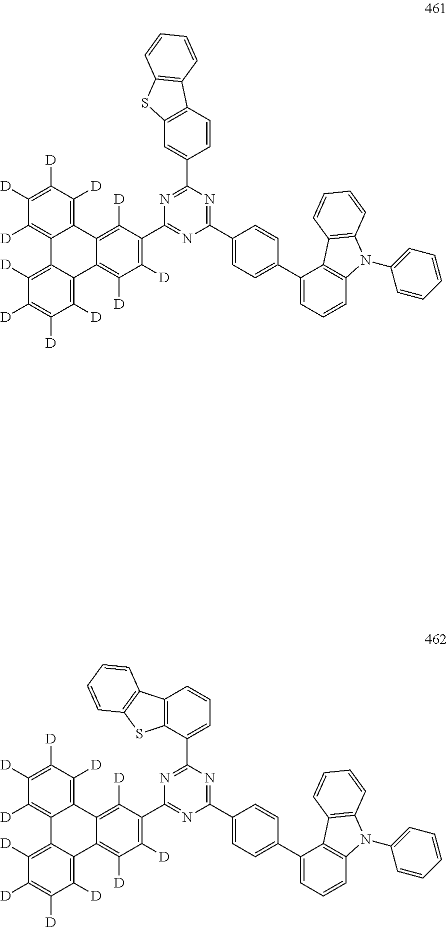 [SMILES: [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c5c5ccccc5n6-c5ccccc5)cc4)nc(-c4cccc5c4sc4ccccc45)n3)c([2H])c([2H])c21]

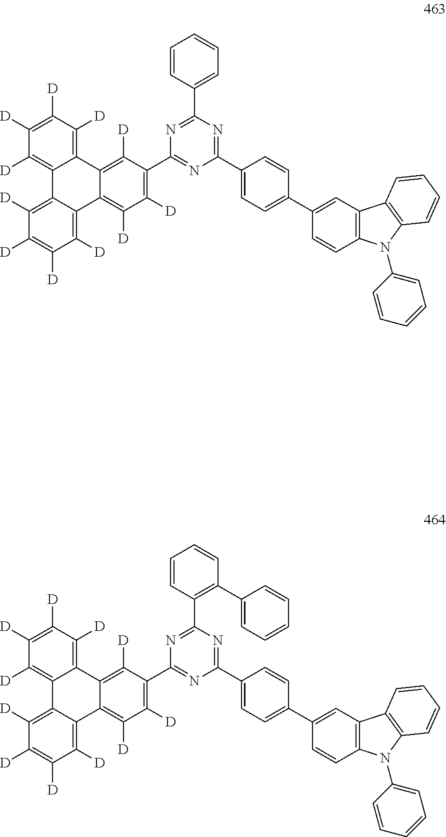 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5ccc6c(c5)c5ccccc5n6-c5ccccc5)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4ccccc4-c4ccccc4)n3)c([2H])c([2H])c21]

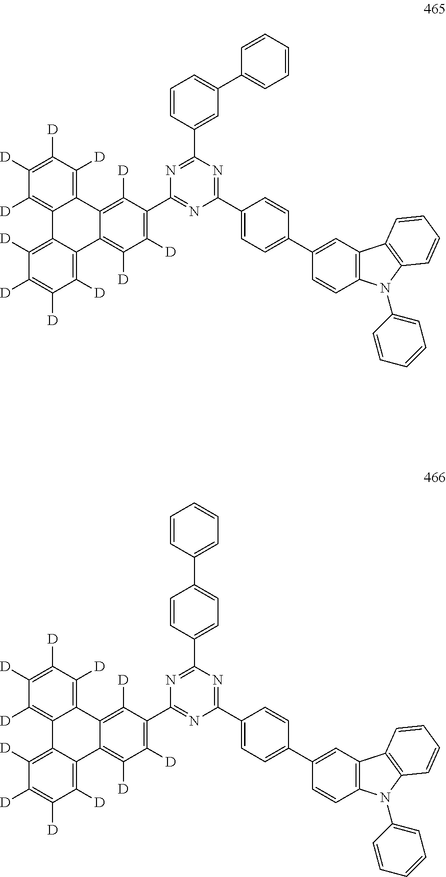 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5ccc6c(c5)c5ccccc5n6-c5ccccc5)cc4)n3)c([2H])c([2H])c21]

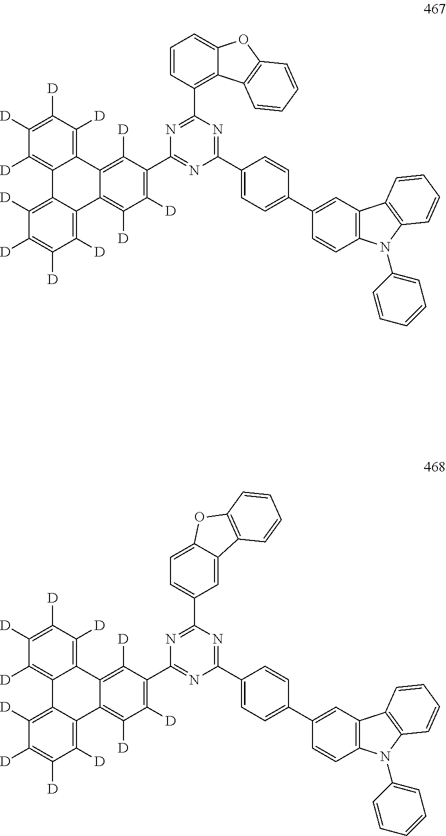 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4ccc5oc6ccccc6c5c4)n3)c([2H])c([2H])c21]

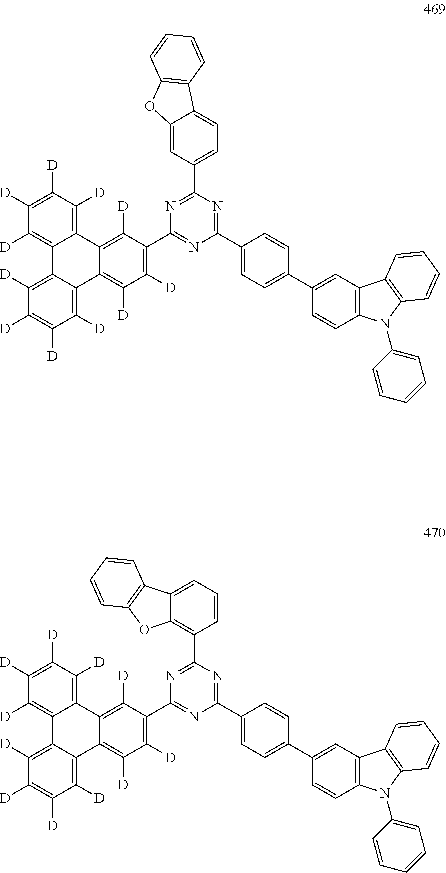 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4ccc5c(c4)oc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4cccc5c4oc4ccccc45)n3)c([2H])c([2H])c21]

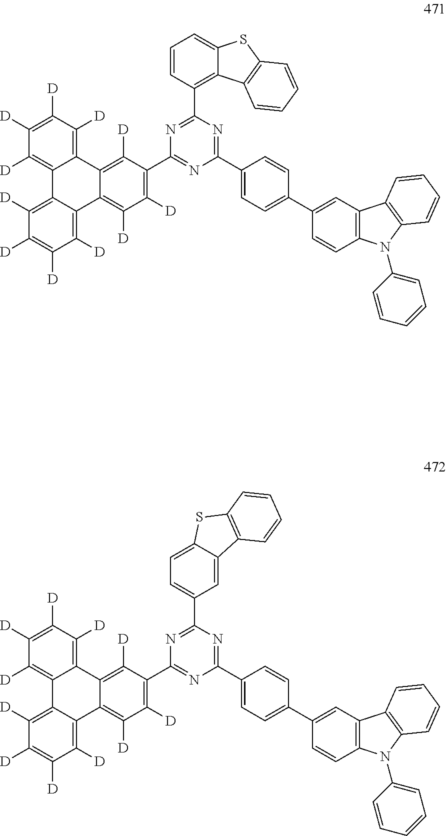 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4cccc5sc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4ccc5sc6ccccc6c5c4)n3)c([2H])c([2H])c21]

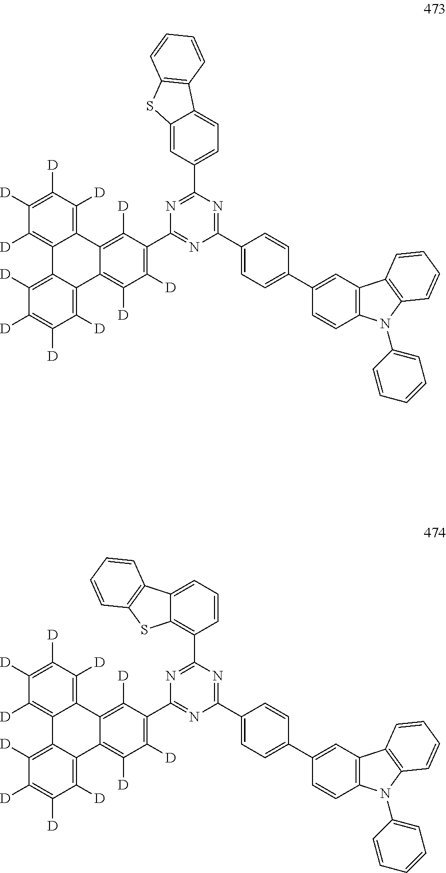 [SMILES: [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4ccc5c(c4)sc4ccccc45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c(c5)c5ccccc5n6-c5ccccc5)cc4)nc(-c4cccc5c4sc4ccccc45)n3)c([2H])c([2H])c21]

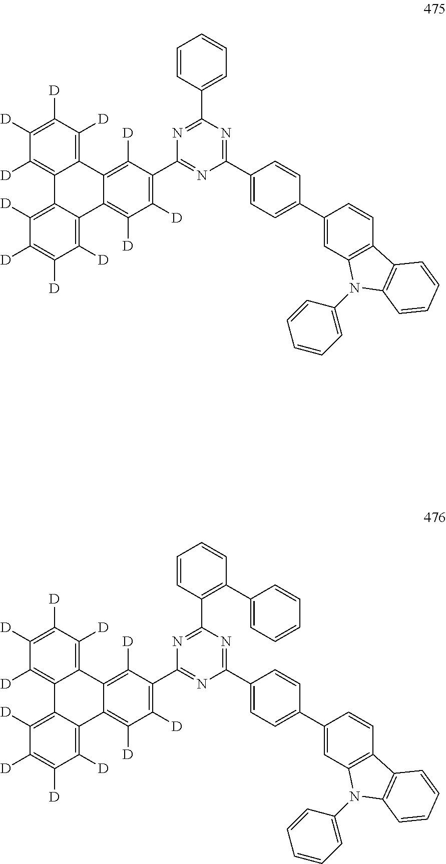 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5ccc6c7ccccc7n(-c7ccccc7)c6c5)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c7ccccc7n(-c7ccccc7)c6c5)cc4)nc(-c4ccccc4-c4ccccc4)n3)c([2H])c([2H])c21]

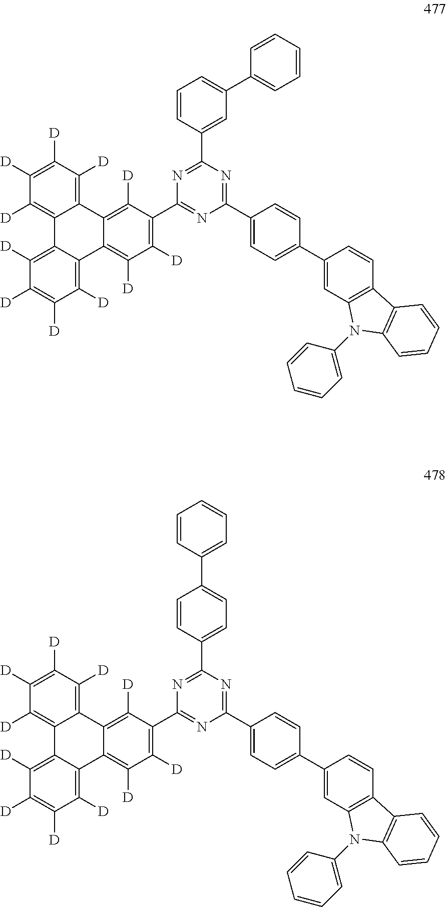 [SMILES: [2H]c1c([2H])c([2H])c2c(c1[2H])c1c([2H])c([2H])c([2H])c([2H])c1c1c([2H])c(-c3nc(-c4ccc(-c5ccc6c7ccccc7n(-c7ccccc7)c6c5)cc4)nc(-c4cccc(-c5ccccc5)c4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccccc5)cc4)nc(-c4ccc(-c5ccc6c7ccccc7n(-c7ccccc7)c6c5)cc4)n3)c([2H])c([2H])c21]

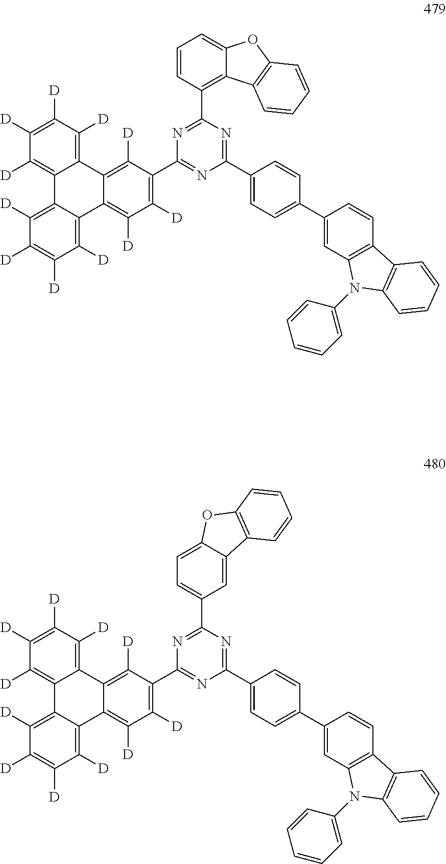 [SMILES: [2H]c1c([2H])c([2H])c2c(c1[2H])c1c([2H])c([2H])c([2H])c([2H])c1c1c([2H])c(-c3nc(-c4ccc(-c5ccc6c7ccccc7n(-c7ccccc7)c6c5)cc4)nc(-c4cccc5oc6ccccc6c45)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5ccc6c7ccccc7n(-c7ccccc7)c6c5)cc4)nc(-c4ccc5oc6ccccc6c5c4)n3)c([2H])c([2H])c21]

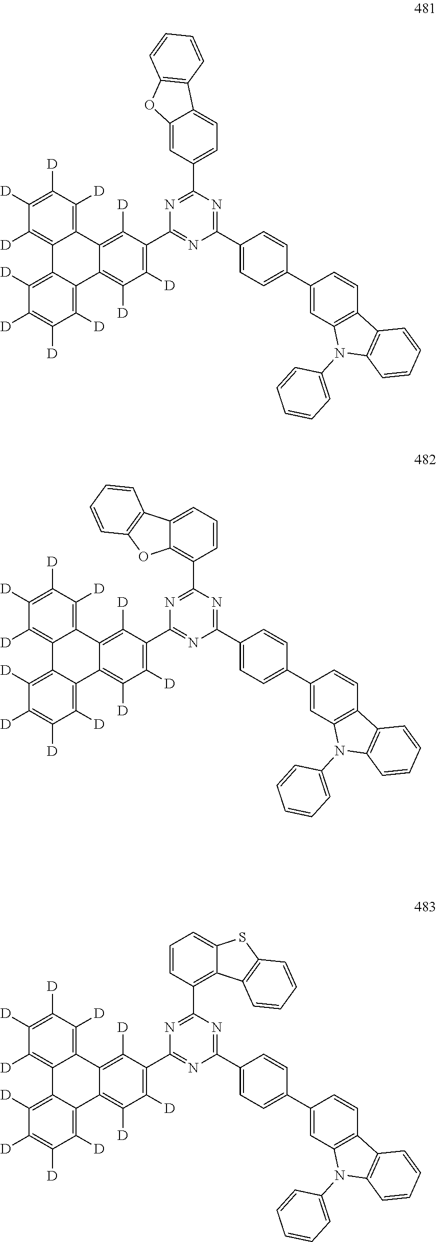

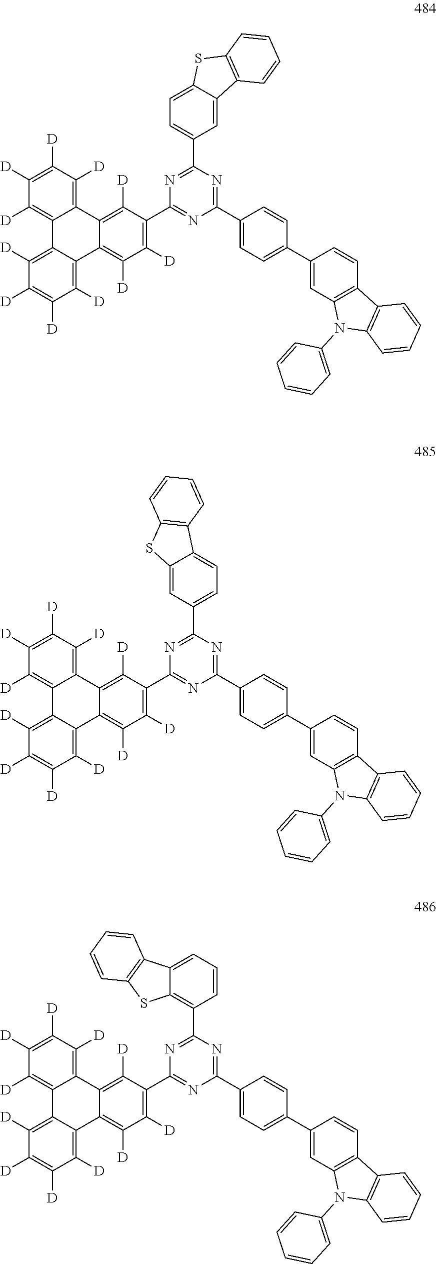

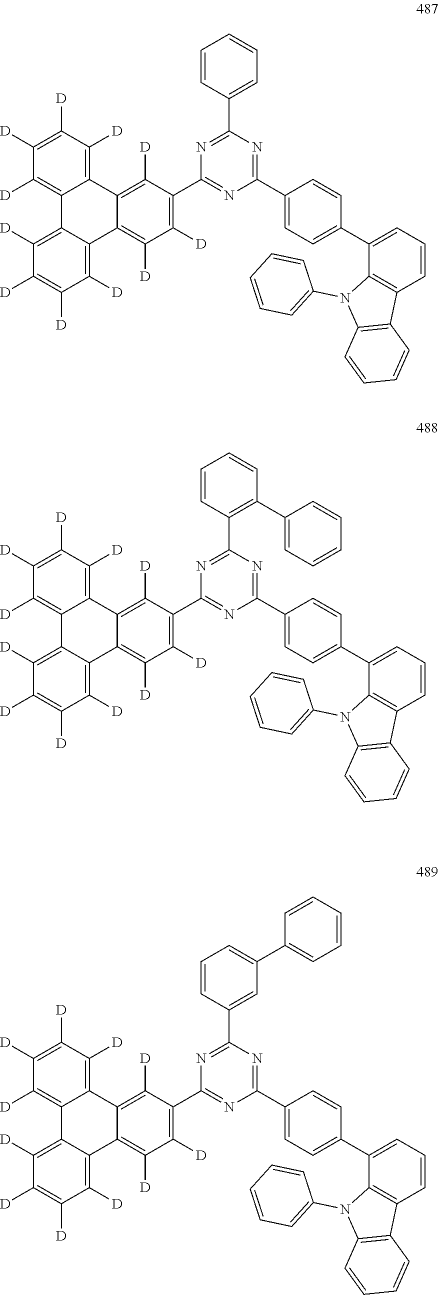 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc(-c5cccc6c7ccccc7n(-c7ccccc7)c56)cc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c7ccccc7n(-c7ccccc7)c56)cc4)nc(-c4ccccc4-c4ccccc4)n3)c([2H])c([2H])c21 ; [2H]c1c([2H])c([2H])c2c(c1[2H])c1c([2H])c([2H])c([2H])c([2H])c1c1c([2H])c(-c3nc(-c4ccc(-c5cccc6c7ccccc7n(-c7ccccc7)c56)cc4)nc(-c4cccc(-c5ccccc5)c4)n3)c([2H])c([2H])c21]

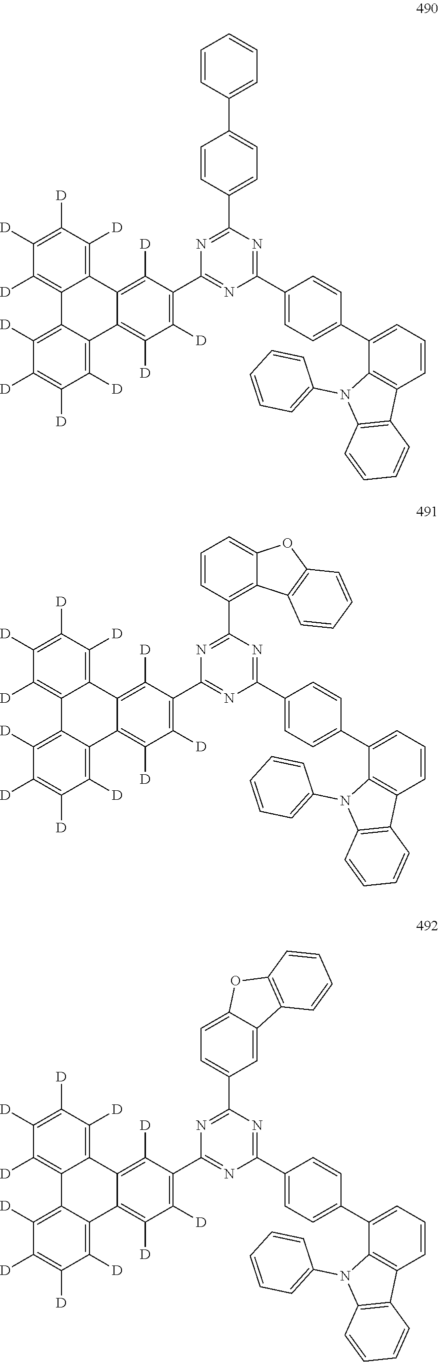

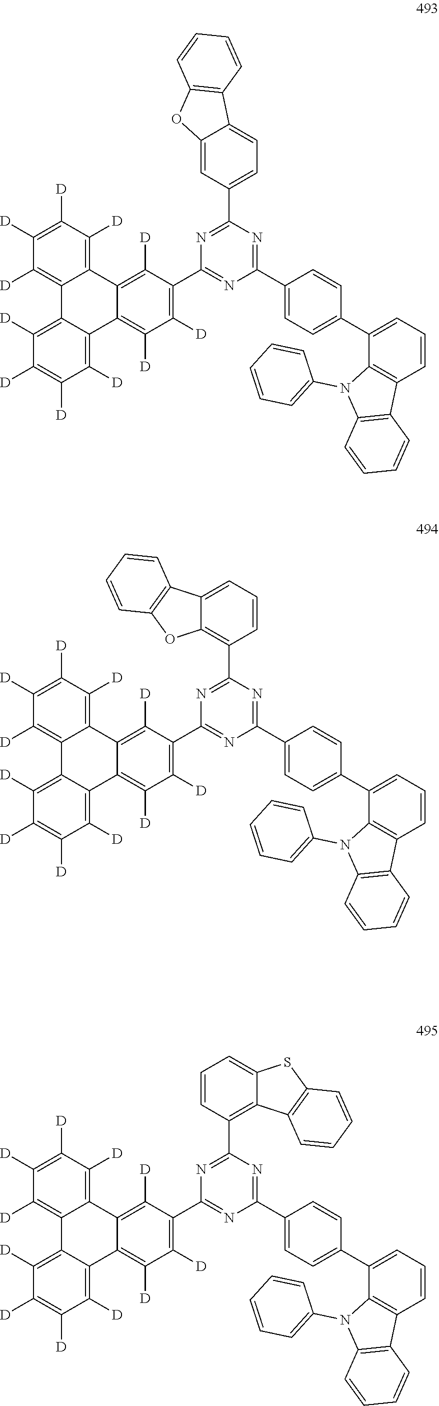

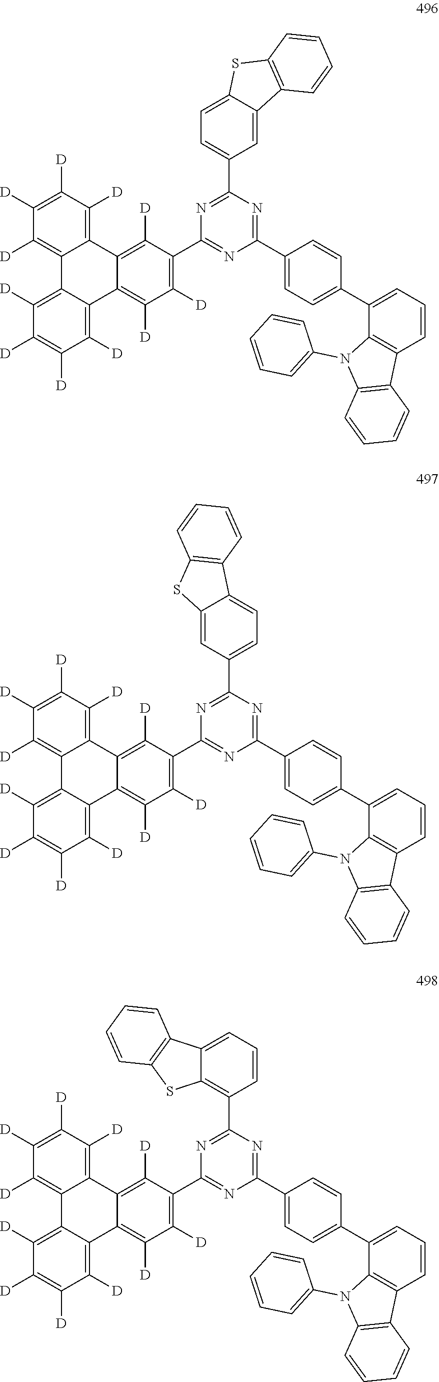

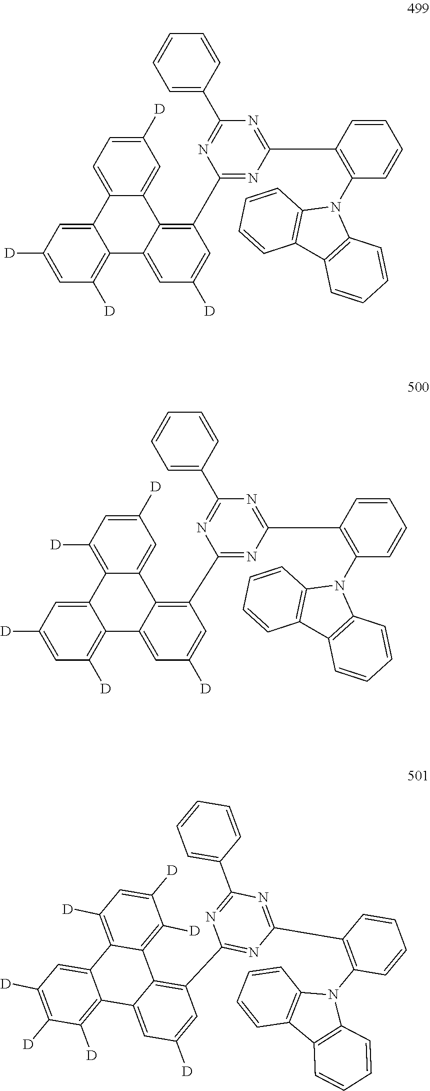 [SMILES: [2H]c1cc([2H])c2c(c1)c1ccc([2H])cc1c1c(-c3nc(-c4ccccc4)nc(-c4ccccc4-n4c5ccccc5c5ccccc54)n3)cc([2H])cc21 ; [2H]c1cc([2H])c2c(c1)c1c([2H])cc([2H])cc1c1c(-c3nc(-c4ccccc4)nc(-c4ccccc4-n4c5ccccc5c5ccccc54)n3)cc([2H])cc21 ; [2H]c1cc(-c2nc(-c3ccccc3)nc(-c3ccccc3-n3c4ccccc4c4ccccc43)n2)c2c(c1)c1c([2H])c([2H])c([2H])cc1c1c([2H])cc([2H])c([2H])c12]

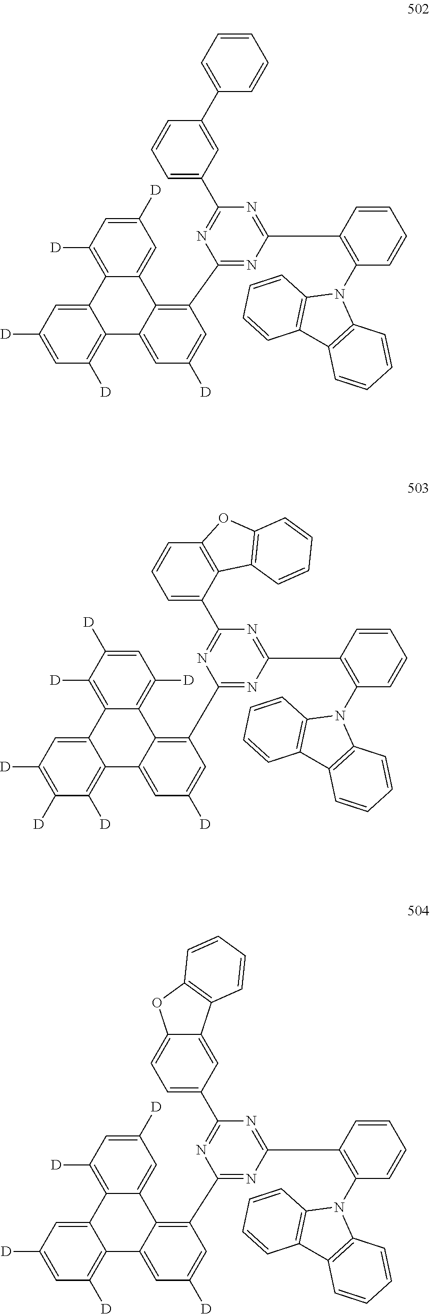 [SMILES: [2H]c1cc([2H])c2c(c1)c1c([2H])cc([2H])cc1c1c(-c3nc(-c4cccc(-c5ccccc5)c4)nc(-c4ccccc4-n4c5ccccc5c5ccccc54)n3)cc([2H])cc21 ; [2H]c1cc(-c2nc(-c3ccccc3-n3c4ccccc4c4ccccc43)nc(-c3cccc4oc5ccccc5c34)n2)c2c(c1)c1c([2H])c([2H])c([2H])cc1c1c([2H])c([2H])cc([2H])c21 ; [2H]c1cc([2H])c2c(c1)c1c([2H])cc([2H])cc1c1c(-c3nc(-c4ccc5oc6ccccc6c5c4)nc(-c4ccccc4-n4c5ccccc5c5ccccc54)n3)cc([2H])cc21]

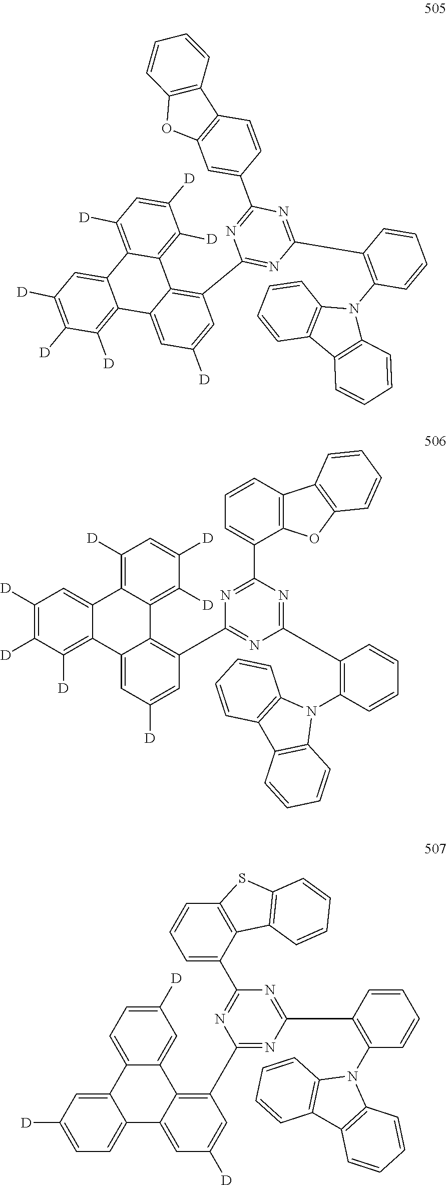

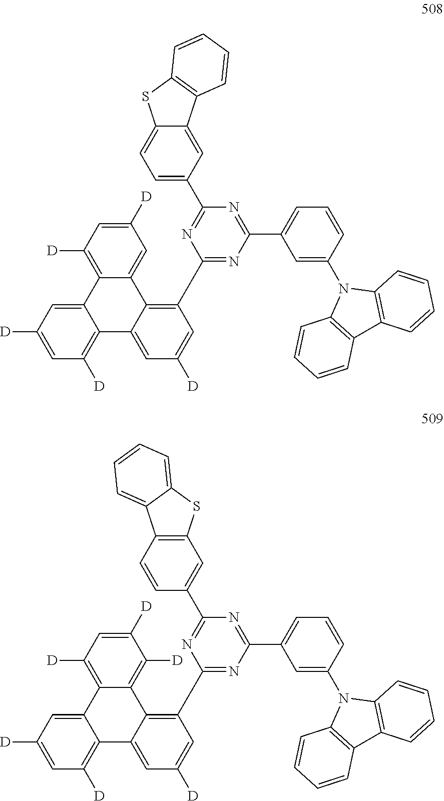 [SMILES: [2H]c1cc([2H])c2c(c1)c1c([2H])cc([2H])cc1c1c(-c3nc(-c4cccc(-n5c6ccccc6c6ccccc65)c4)nc(-c4ccc5sc6ccccc6c5c4)n3)cc([2H])cc21 ; [2H]c1cc([2H])c2c(c1)c1c([2H])cc([2H])c([2H])c1c1c(-c3nc(-c4cccc(-n5c6ccccc6c6ccccc65)c4)nc(-c4ccc5c(c4)sc4ccccc45)n3)cc([2H])cc21]

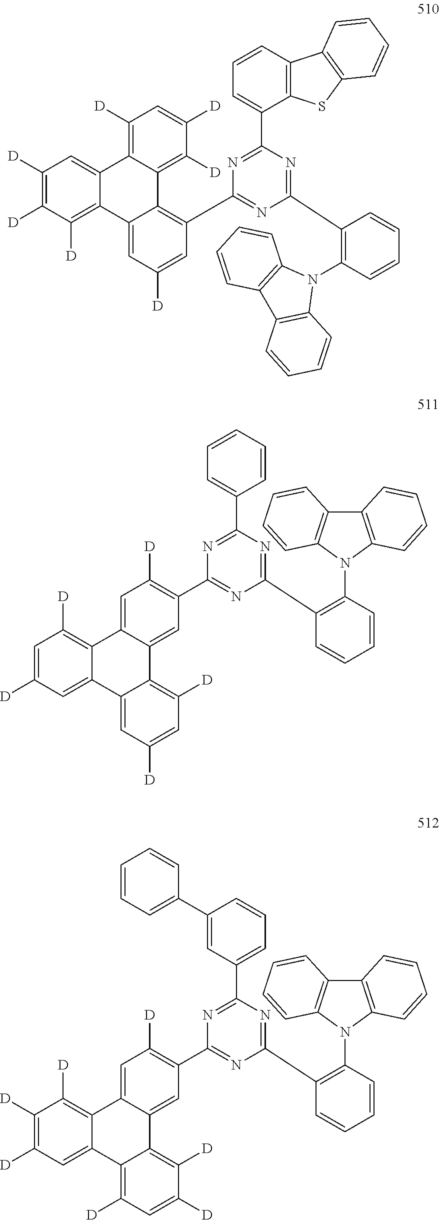 [SMILES: [2H]c1cc(-c2nc(-c3ccccc3-n3c4ccccc4c4ccccc43)nc(-c3cccc4c3sc3ccccc34)n2)c2c(c1)c1c([2H])c([2H])c([2H])cc1c1c([2H])cc([2H])c([2H])c12 ; [2H]c1cc([2H])c2c(c1)c1cc([2H])cc([2H])c1c1cc(-c3nc(-c4ccccc4)nc(-c4ccccc4-n4c5ccccc5c5ccccc54)n3)c([2H])cc12 ; [2H]c1cc2c(cc1-c1nc(-c3cccc(-c4ccccc4)c3)nc(-c3ccccc3-n3c4ccccc4c4ccccc43)n1)c1c([2H])c([2H])cc([2H])c1c1cc([2H])c([2H])c([2H])c21]

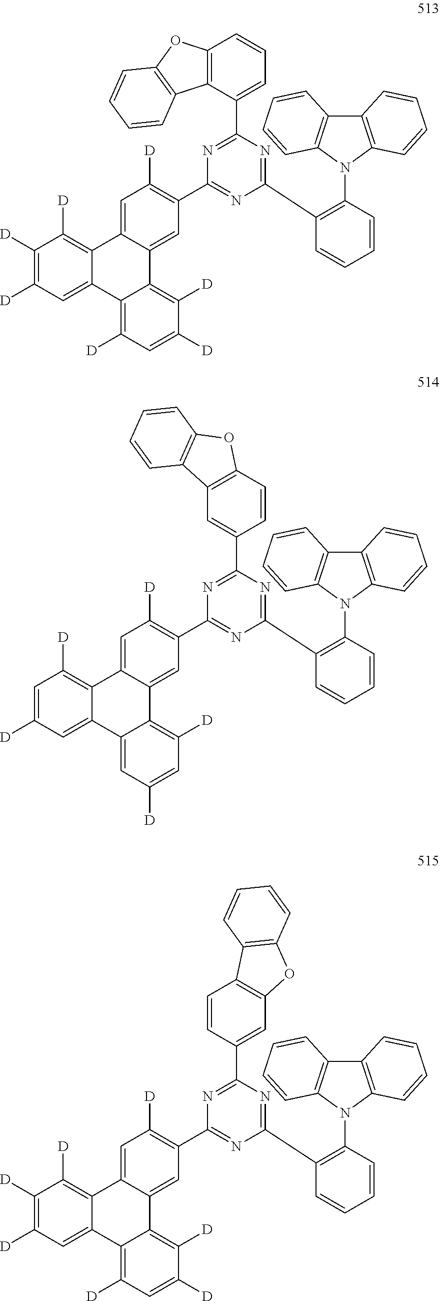 [SMILES: [2H]c1cc2c(cc1-c1nc(-c3ccccc3-n3c4ccccc4c4ccccc43)nc(-c3cccc4oc5ccccc5c34)n1)c1c([2H])c([2H])cc([2H])c1c1cc([2H])c([2H])c([2H])c21 ; [2H]c1cc([2H])c2c(c1)c1cc([2H])cc([2H])c1c1cc(-c3nc(-c4ccc5oc6ccccc6c5c4)nc(-c4ccccc4-n4c5ccccc5c5ccccc54)n3)c([2H])cc12 ; [2H]c1cc2c(cc1-c1nc(-c3ccc4c(c3)oc3ccccc34)nc(-c3ccccc3-n3c4ccccc4c4ccccc43)n1)c1c([2H])c([2H])cc([2H])c1c1cc([2H])c([2H])c([2H])c21]

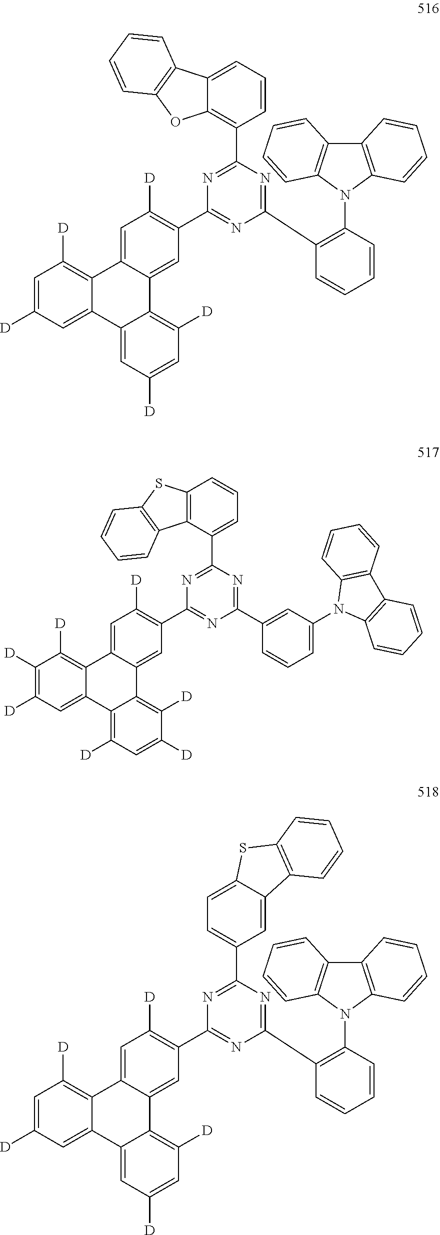 [SMILES: [2H]c1cc([2H])c2c(c1)c1cc([2H])cc([2H])c1c1cc(-c3nc(-c4ccccc4-n4c5ccccc5c5ccccc54)nc(-c4cccc5c4oc4ccccc45)n3)c([2H])cc12 ; [2H]c1cc2c(cc1-c1nc(-c3cccc(-n4c5ccccc5c5ccccc54)c3)nc(-c3cccc4sc5ccccc5c34)n1)c1c([2H])c([2H])cc([2H])c1c1cc([2H])c([2H])c([2H])c21 ; [2H]c1cc([2H])c2c(c1)c1cc([2H])cc([2H])c1c1cc(-c3nc(-c4ccc5sc6ccccc6c5c4)nc(-c4ccccc4-n4c5ccccc5c5ccccc54)n3)c([2H])cc12]

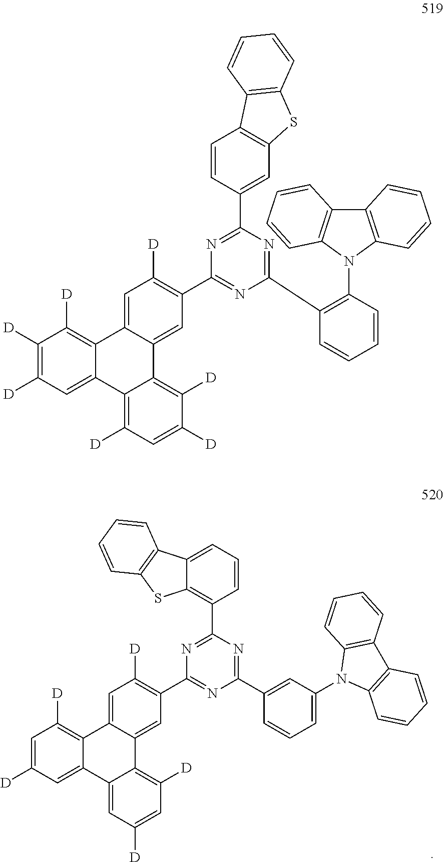 [SMILES: [2H]c1cc2c(cc1-c1nc(-c3ccc4c(c3)sc3ccccc34)nc(-c3ccccc3-n3c4ccccc4c4ccccc43)n1)c1c([2H])c([2H])cc([2H])c1c1cc([2H])c([2H])c([2H])c21 ; [2H]c1cc([2H])c2c(c1)c1cc([2H])cc([2H])c1c1cc(-c3nc(-c4cccc(-n5c6ccccc6c6ccccc65)c4)nc(-c4cccc5c4sc4ccccc45)n3)c([2H])cc12]

9. An organic light emitting device comprising:
a first electrode;
a second electrode provided to face the first electrode; and
an organic material layer having one or more layers provided between the first electrode and the second electrode,
wherein one or more layers of the organic material layer comprise the heterocyclic compound of claim 1.

10. The organic light emitting device of claim 9, wherein one or more layers of the organic material layer further comprise a heterocyclic compound represented by the following Chemical Formula 2 or 3: 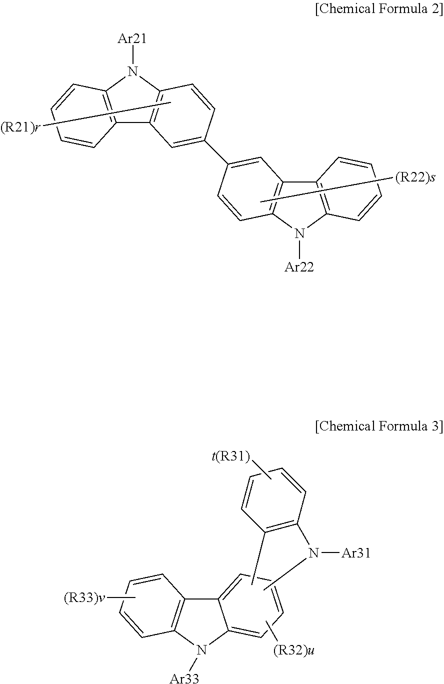
in Chemical Formulae 2 and 3,
R21, R22, and R31 to R33 are the same as or different from each other, and are each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR′R″R′″; —P(═O)R′R″; and an amine group which is unsubstituted or substituted with a substituted or unsubstituted C1 to C60 alkyl group, a substituted or unsubstituted C6 to C60 aryl group or a substituted or unsubstituted C2 to C60 heteroaryl group, or two or more adjacent groups are bonded to each other to form a substituted or unsubstituted C6 to C60 aliphatic or aromatic hydrocarbon ring or a substituted or unsubstituted C2 to C60 aliphatic or aromatic hetero ring,
R′, R″, and R′″ are the same as or different from each other, and are each independently hydrogen; deuterium; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r and s are each an integer from 0 to 7, and when each of r and s is 2 or higher, substituents in the parenthesis are the same as or different from each other,
t and v are each an integer from 0 to 4, and when each of t and v is 2 or higher, substituents in the parenthesis are the same as or different from each other,
u is an integer from 0 to 2, and when u is 2, substituents in the parenthesis are the same as or different from each other, and
Ar21, Ar22, Ar31, and Ar33 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

11. The organic light emitting device of claim 10, wherein the heterocyclic compound represented by Chemical Formula 2 or 3 is any one of the followings:

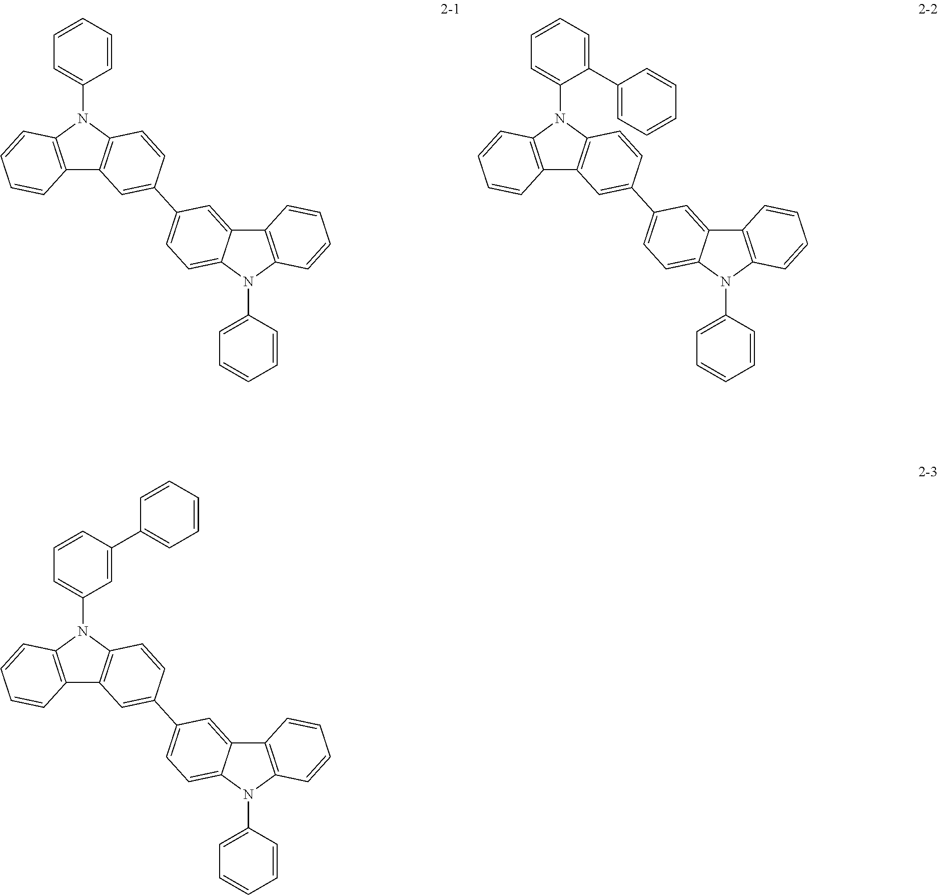 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1]

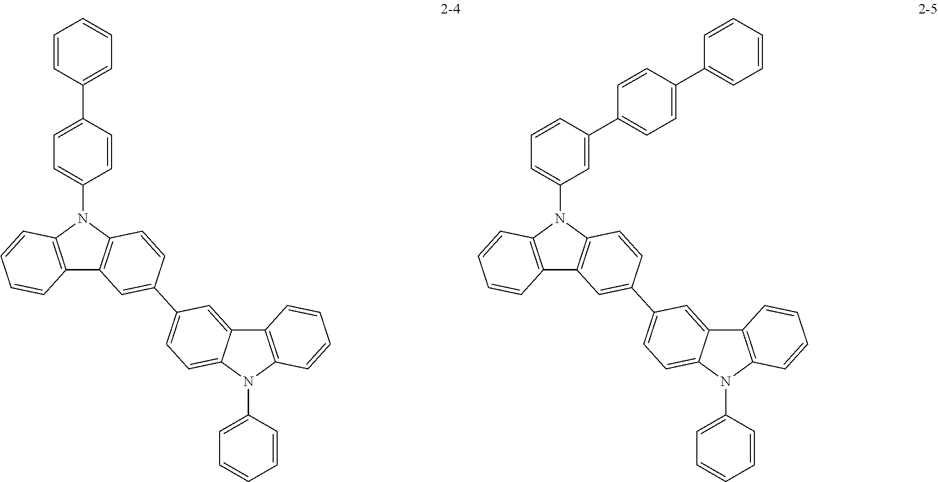 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

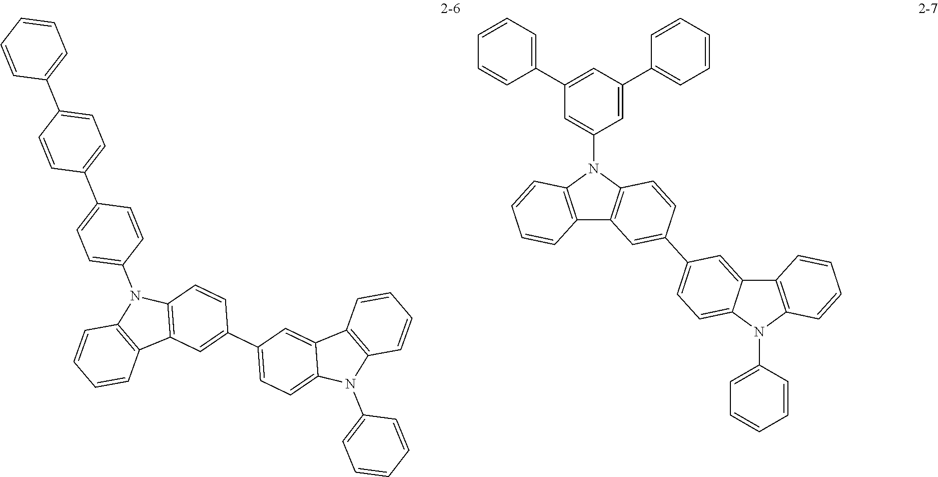 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

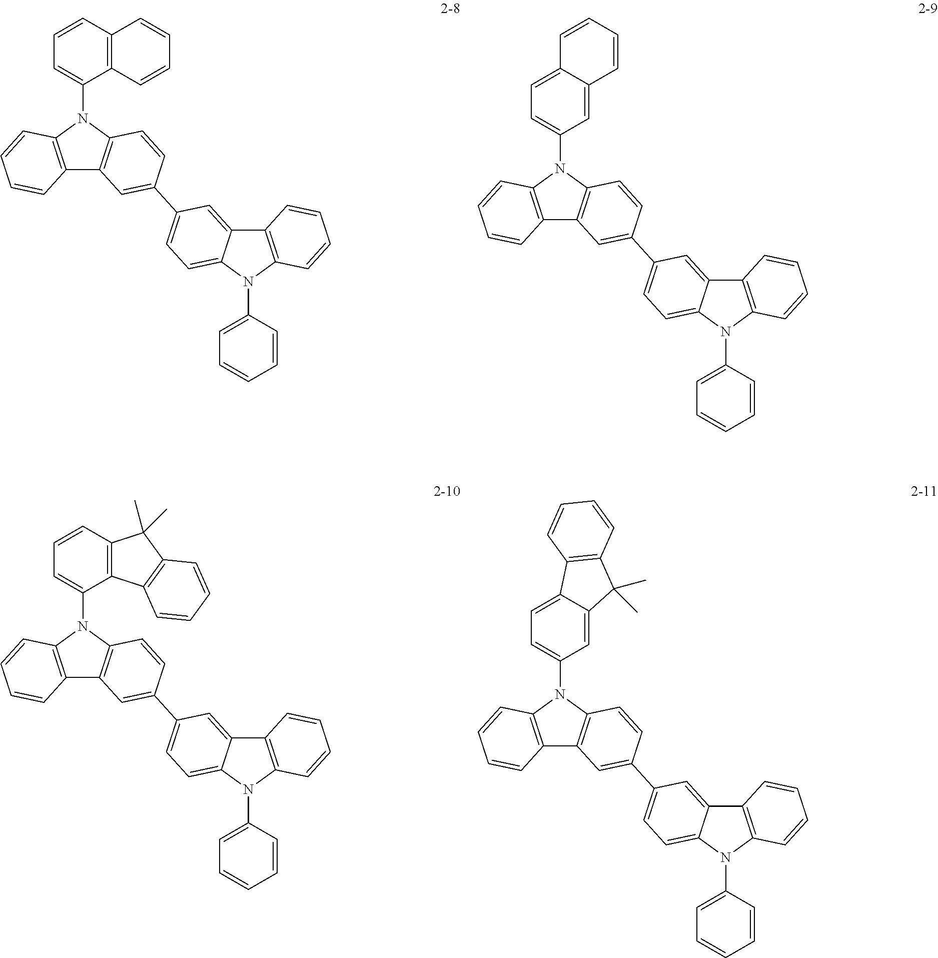 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21]

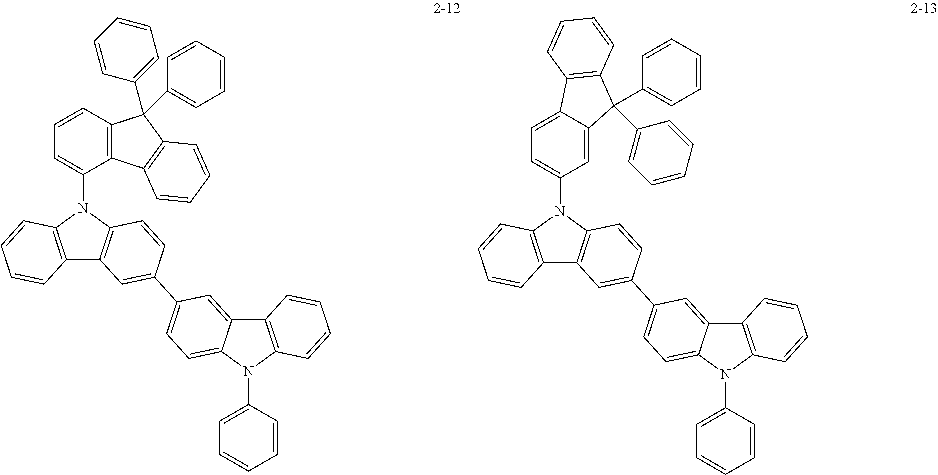 [SMILES: c1ccc(-n2c3ccccc3c3cc(-c4ccc5c(c4)c4ccccc4n5-c4cccc5c4-c4ccccc4C5(c4ccccc4)c4ccccc4)ccc32)cc1 ; c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1]

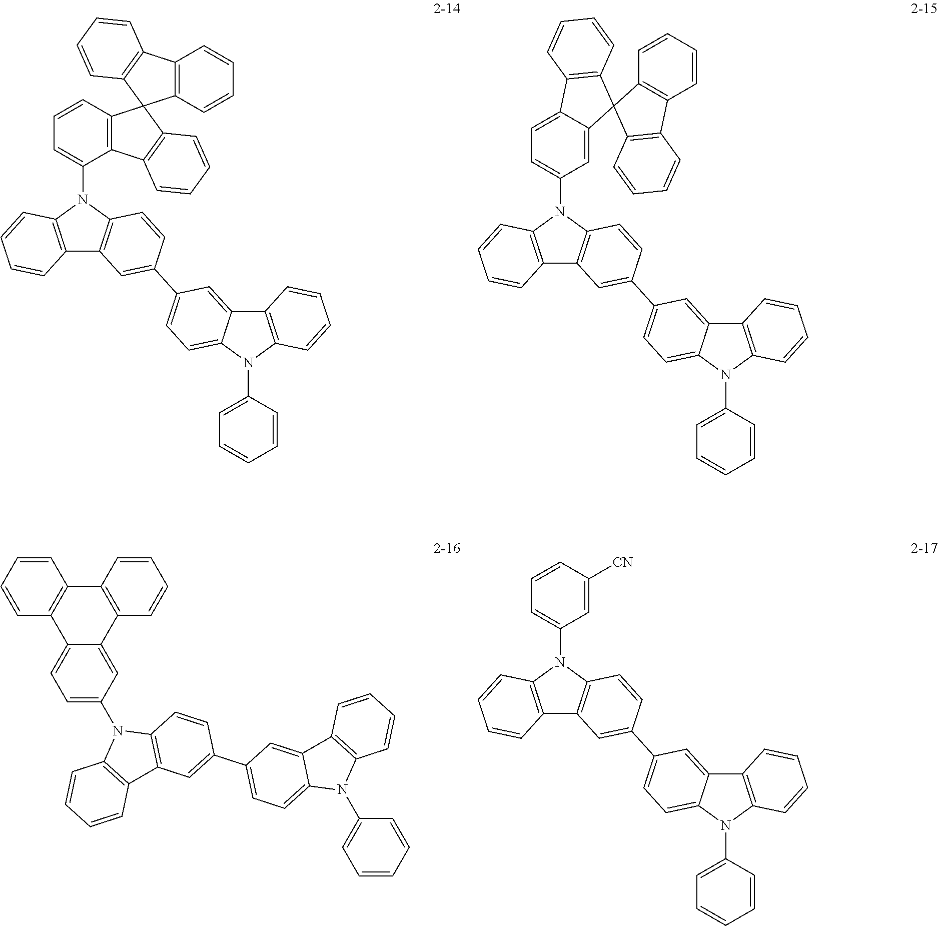 [SMILES: c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1]

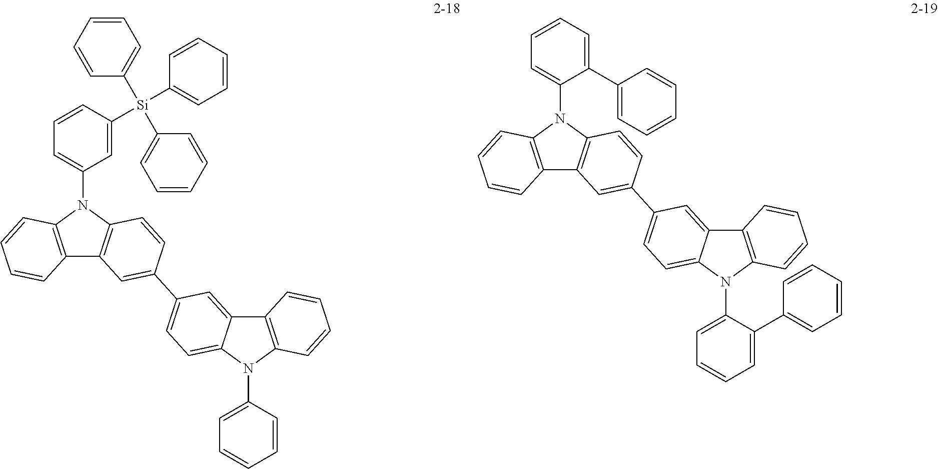 [SMILES: c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1]

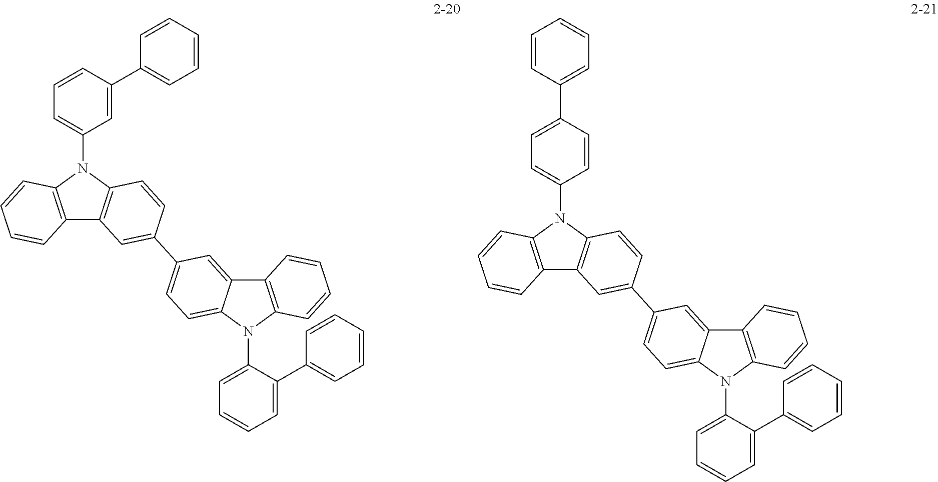 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

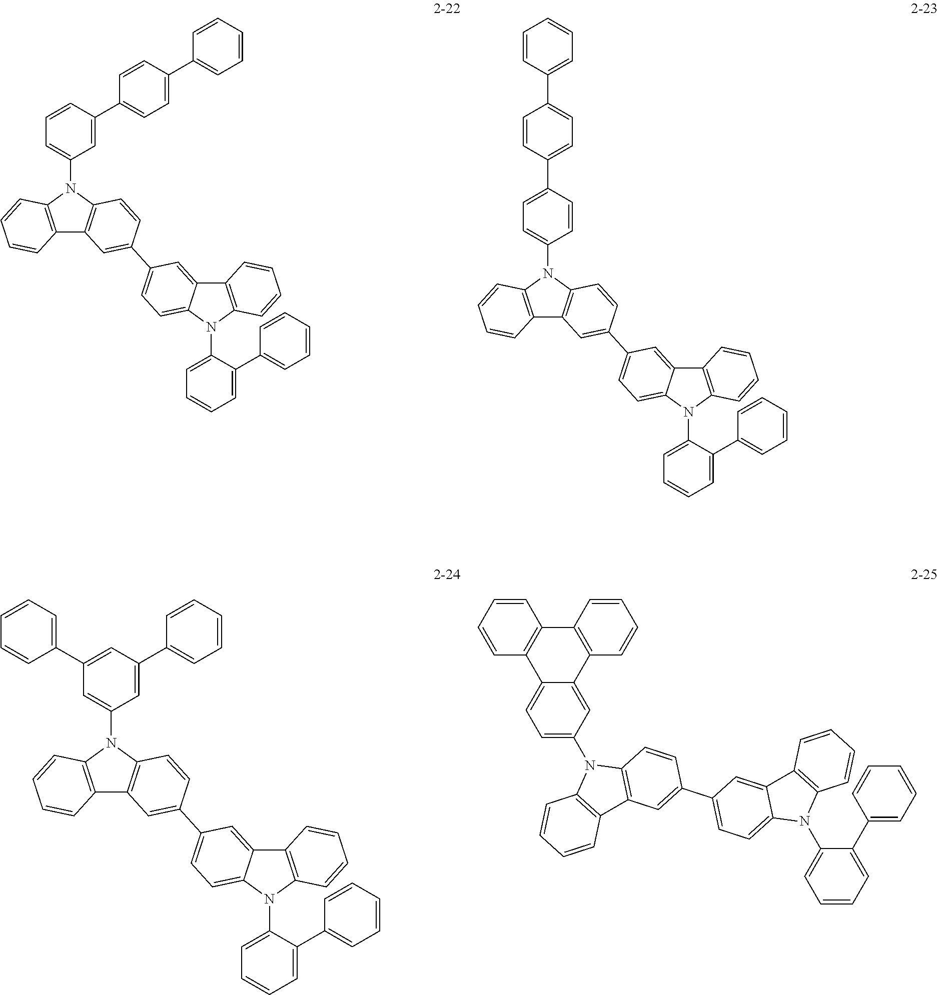 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4ccc5c6ccccc6c6ccccc6c5c4)ccc32)cc1]

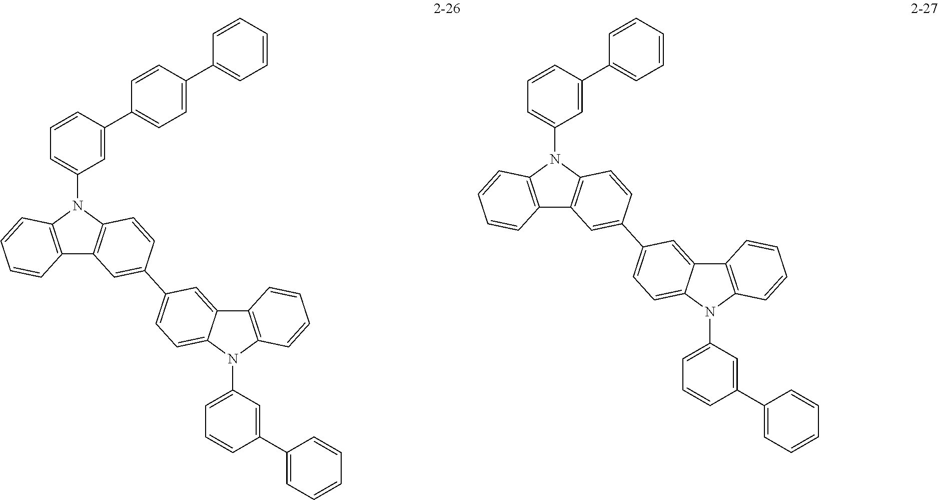 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

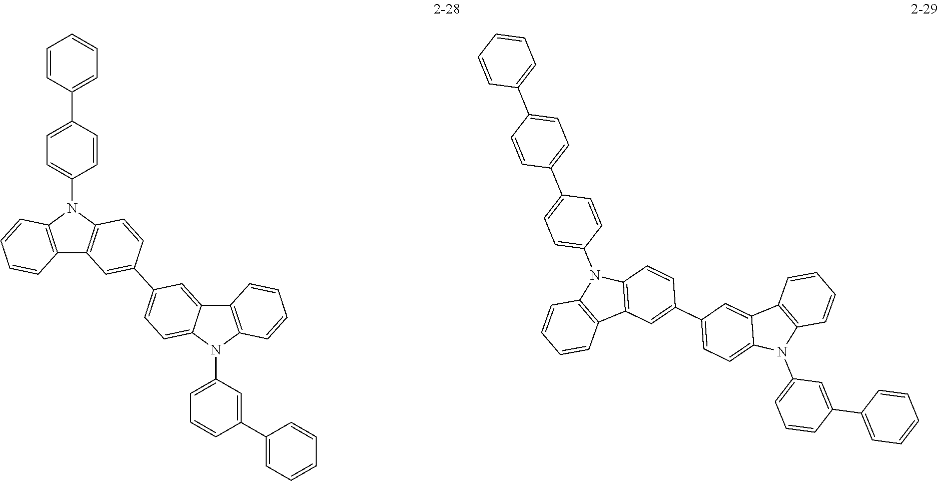 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

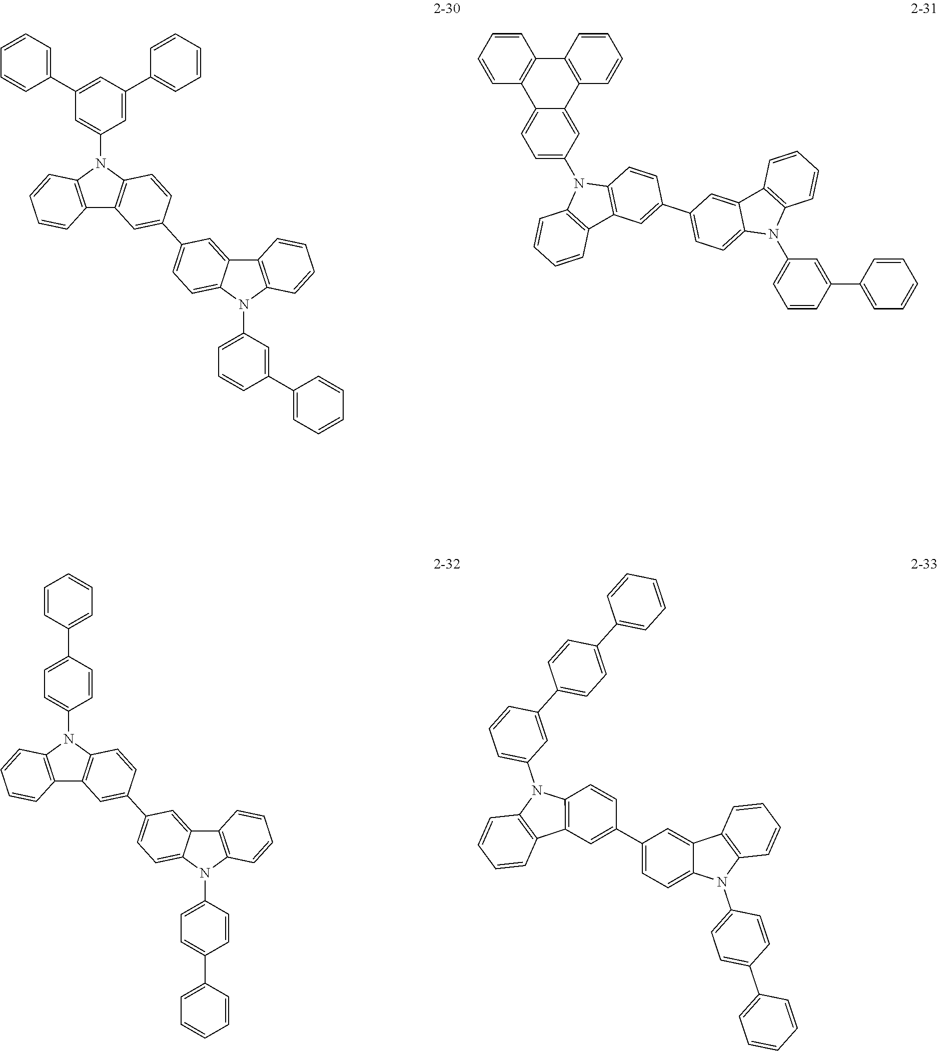 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

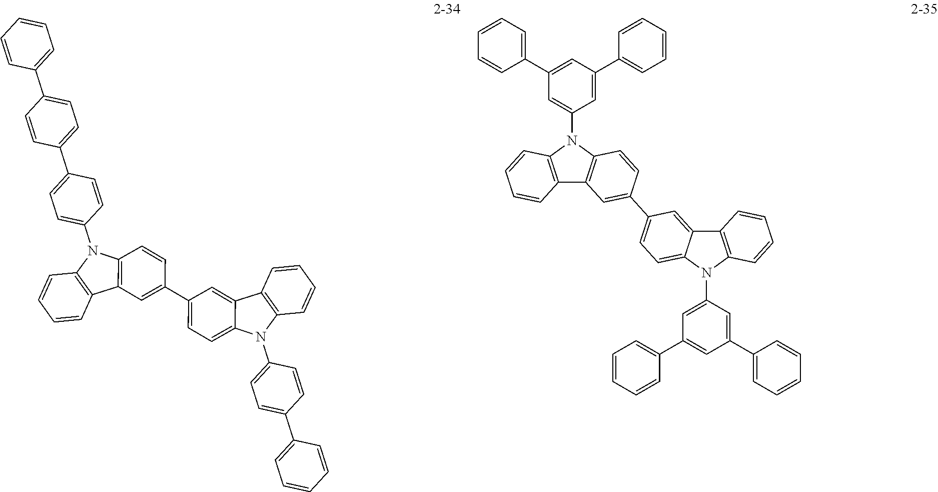 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

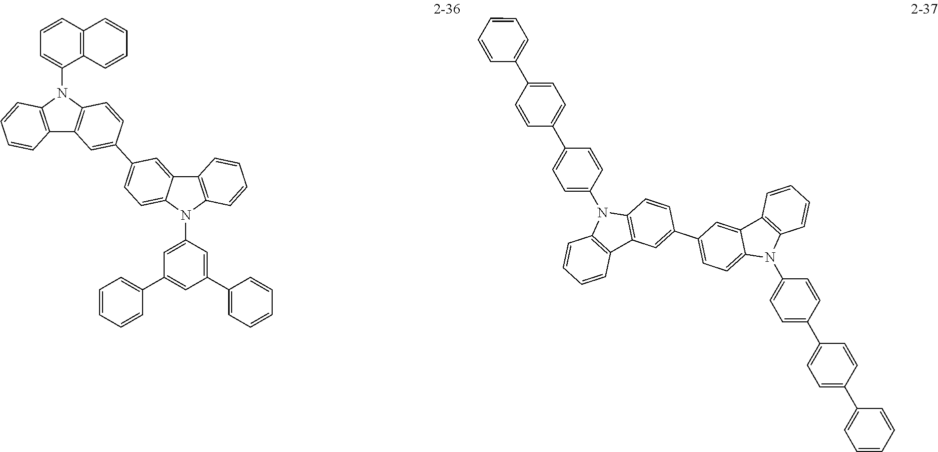 [SMILES: c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1]

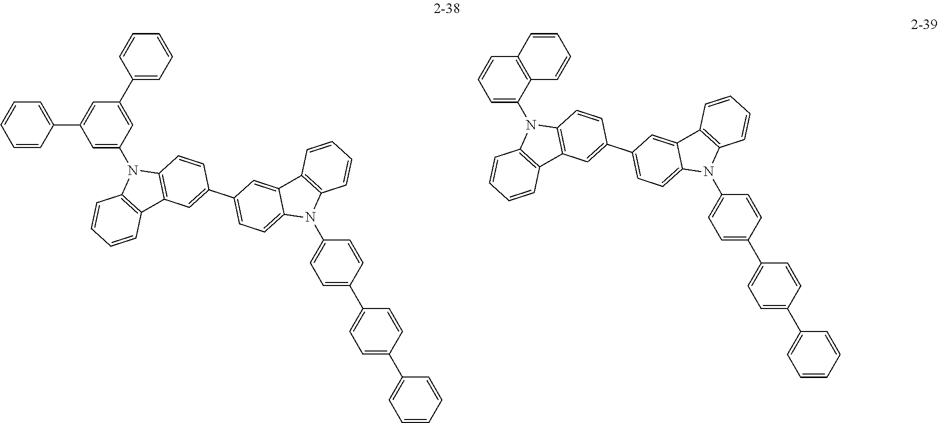 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1]

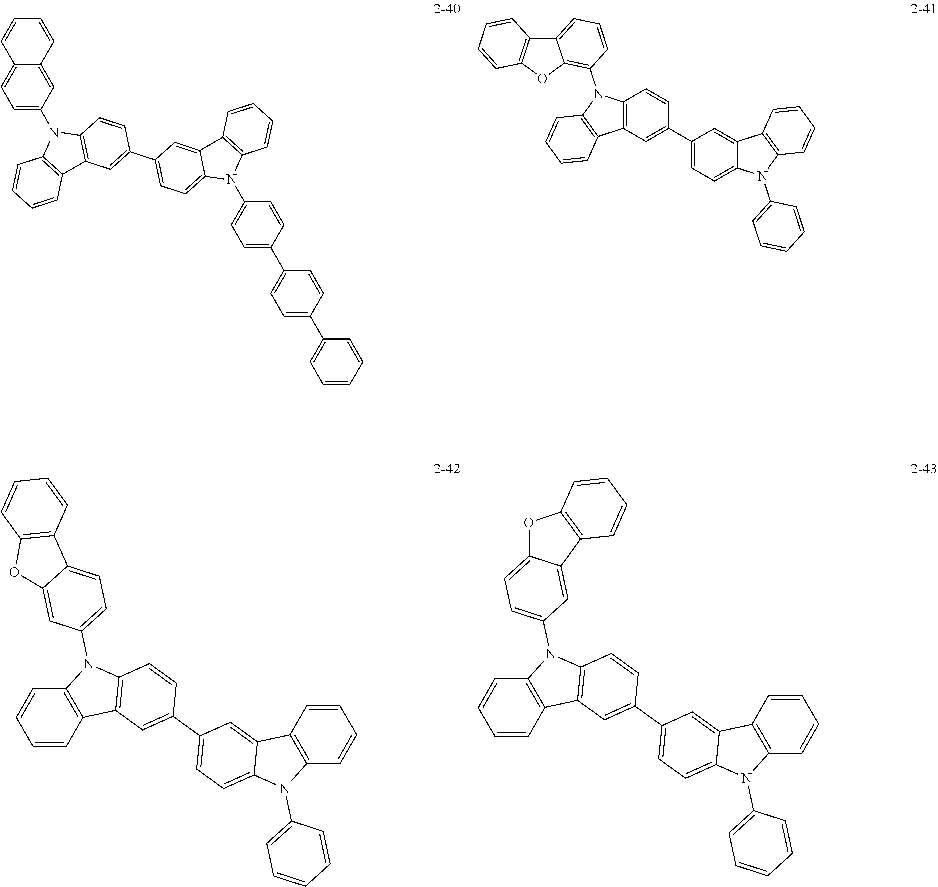 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1]

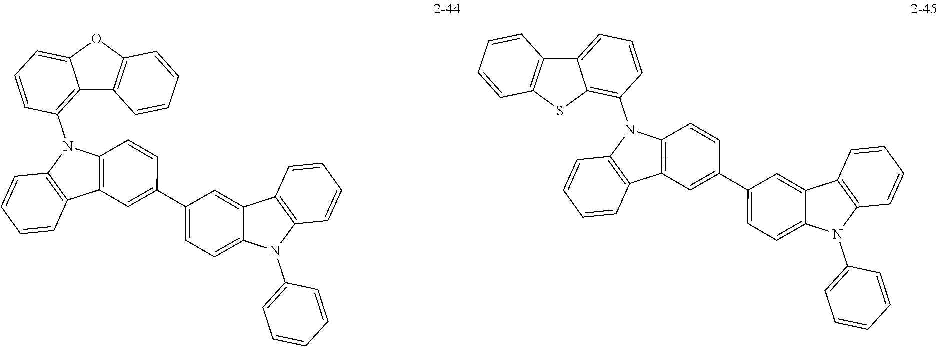 [SMILES: c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4sc4ccccc45)ccc32)cc1]

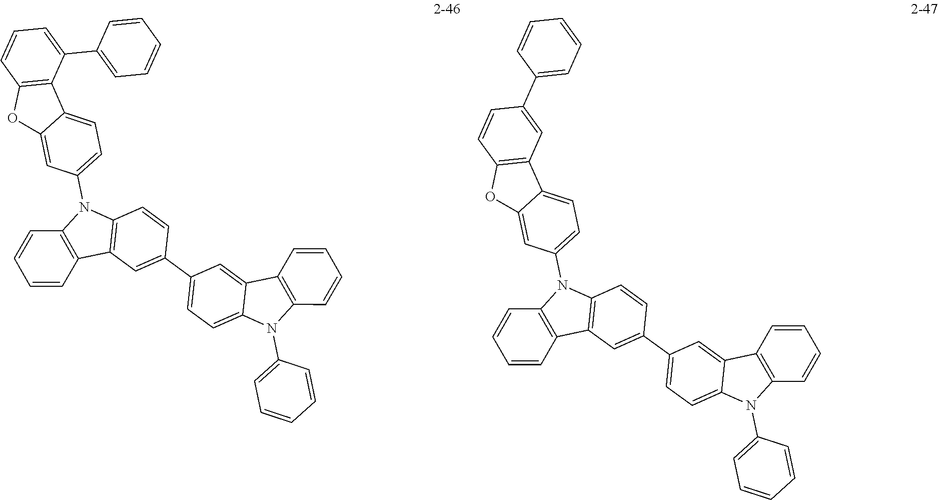 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc3oc4cc(-n5c6ccccc6c6cc(-c7ccc8c(c7)c7ccccc7n8-c7ccccc7)ccc65)ccc4c3c2)cc1]

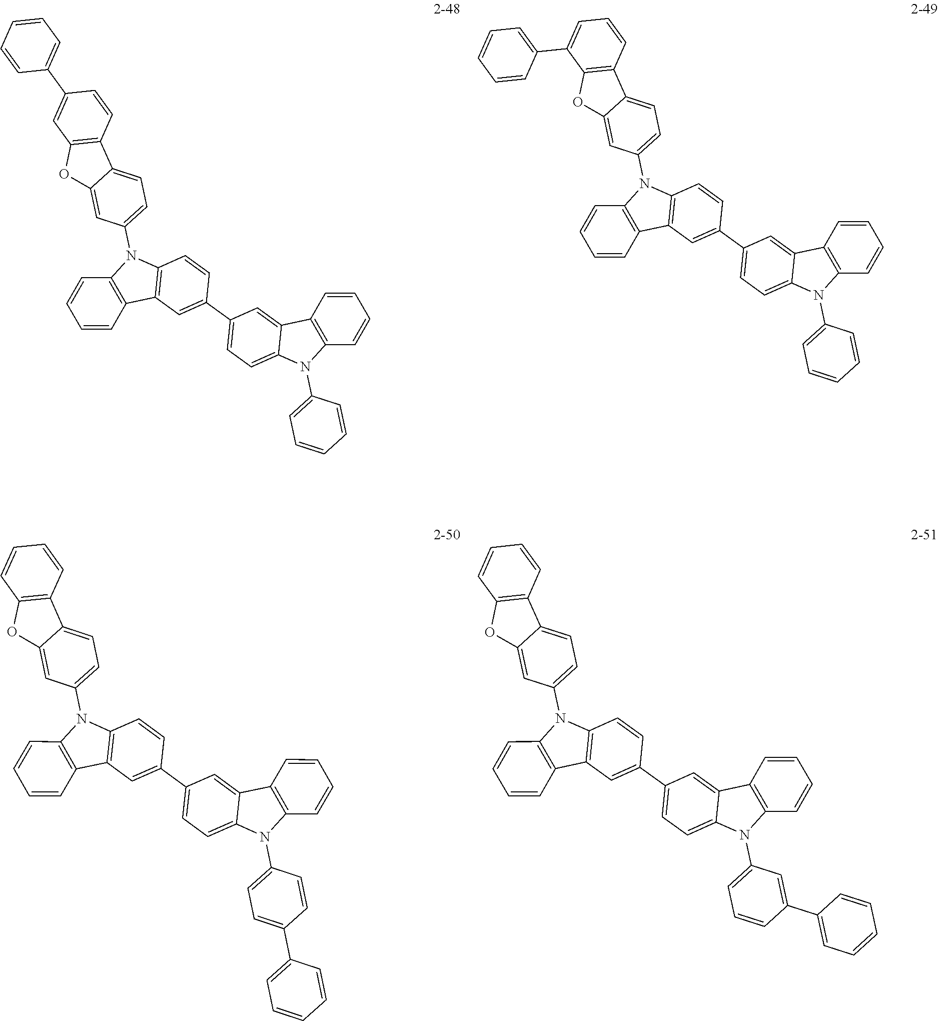 [SMILES: c1ccc(-c2ccc3c(c2)oc2cc(-n4c5ccccc5c5cc(-c6ccc7c(c6)c6ccccc6n7-c6ccccc6)ccc54)ccc23)cc1 ; c1ccc(-c2cccc3c2oc2cc(-n4c5ccccc5c5cc(-c6ccc7c(c6)c6ccccc6n7-c6ccccc6)ccc54)ccc23)cc1 ; c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2cccc(-n3c4ccccc4c4cc(-c5ccc6c(c5)c5ccccc5n6-c5ccc6c(c5)oc5ccccc56)ccc43)c2)cc1]

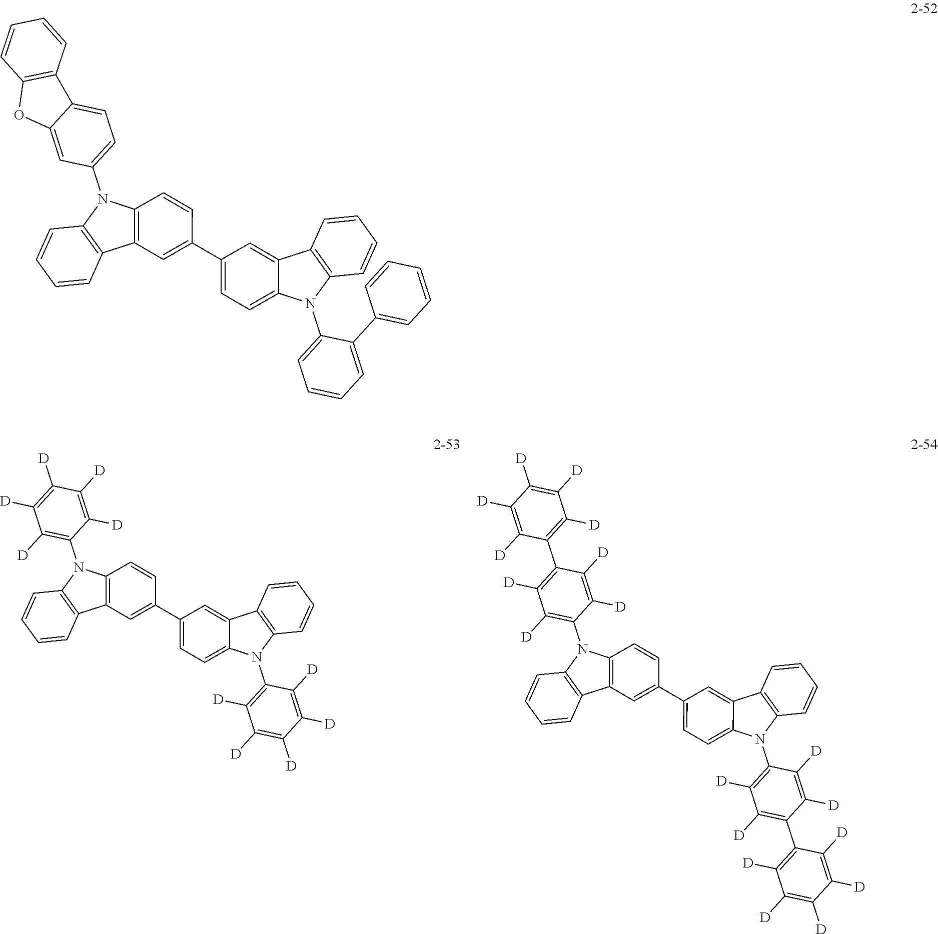 [SMILES: c1ccc(-c2ccccc2-n2c3ccccc3c3cc(-c4ccc5c(c4)c4ccccc4n5-c4ccc5c(c4)oc4ccccc45)ccc32)cc1 ; [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

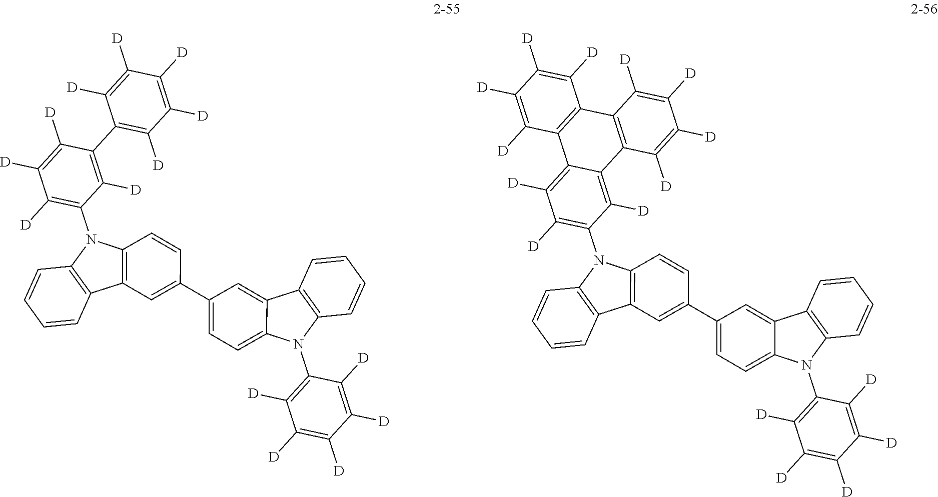 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H] ; [2H]c1c([2H])c([2H])c(-n2c3ccccc3c3cc(-c4ccc5c(c4)c4ccccc4n5-c4c([2H])c([2H])c5c6c([2H])c([2H])c([2H])c([2H])c6c6c([2H])c([2H])c([2H])c([2H])c6c5c4[2H])ccc32)c([2H])c1[2H]]

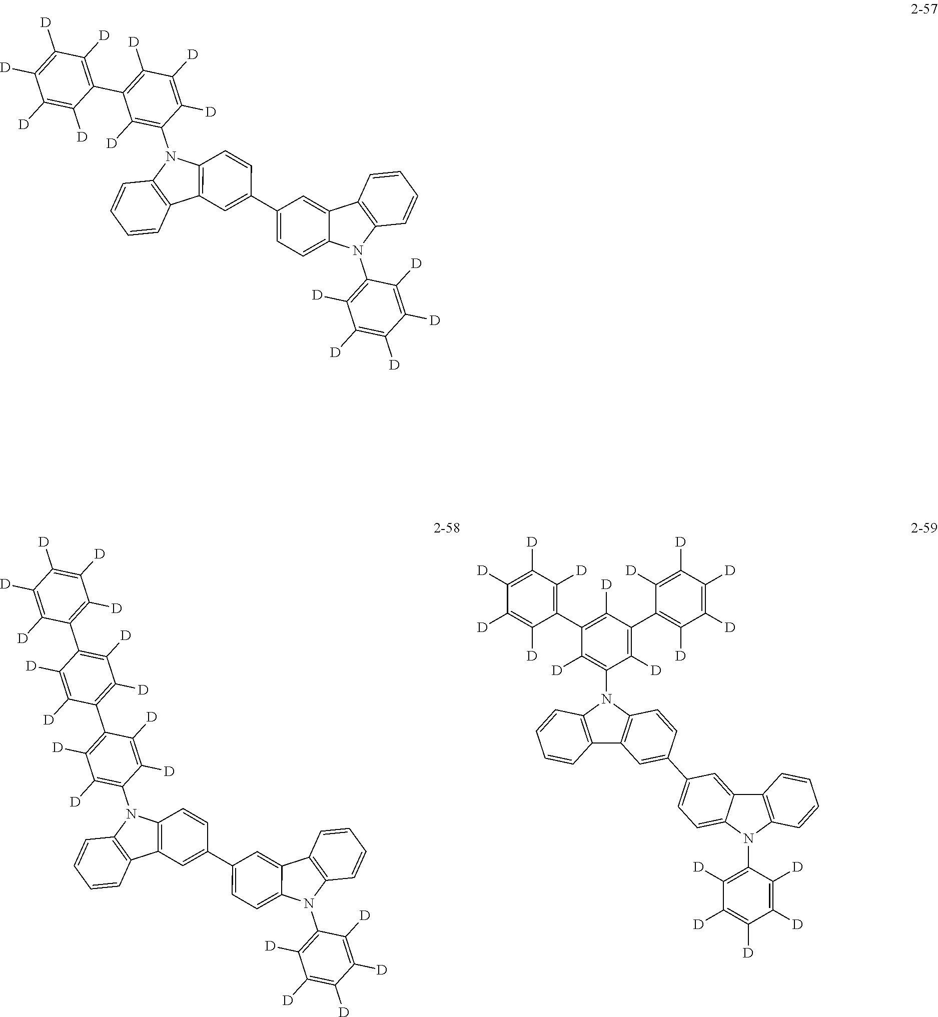

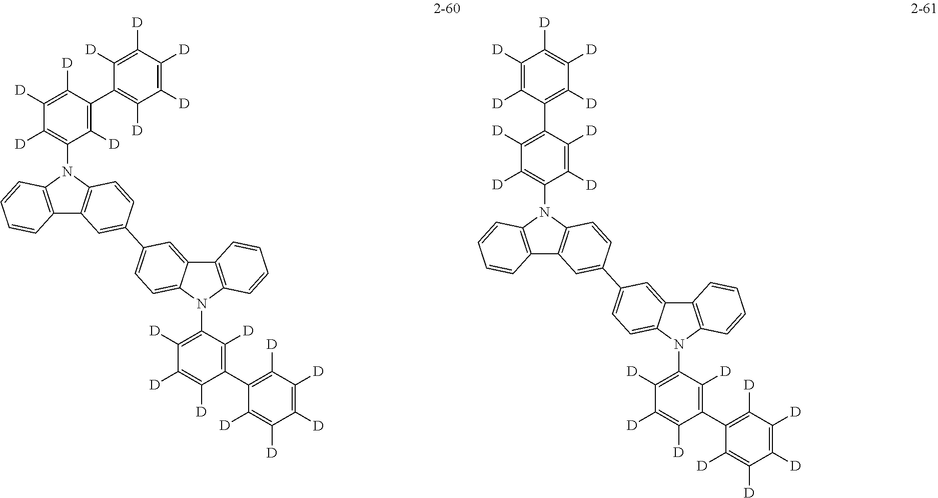 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]]

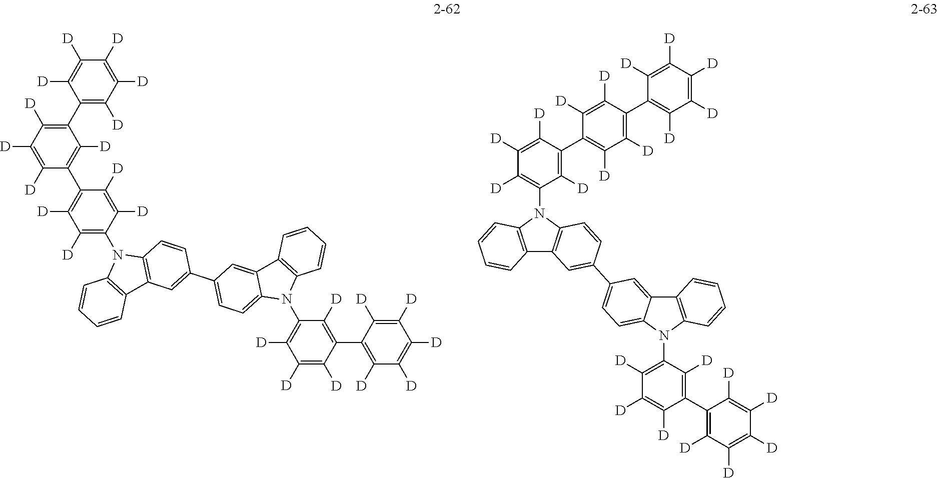 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c3[2H])c([2H])c2[2H])c([2H])c1[2H]]

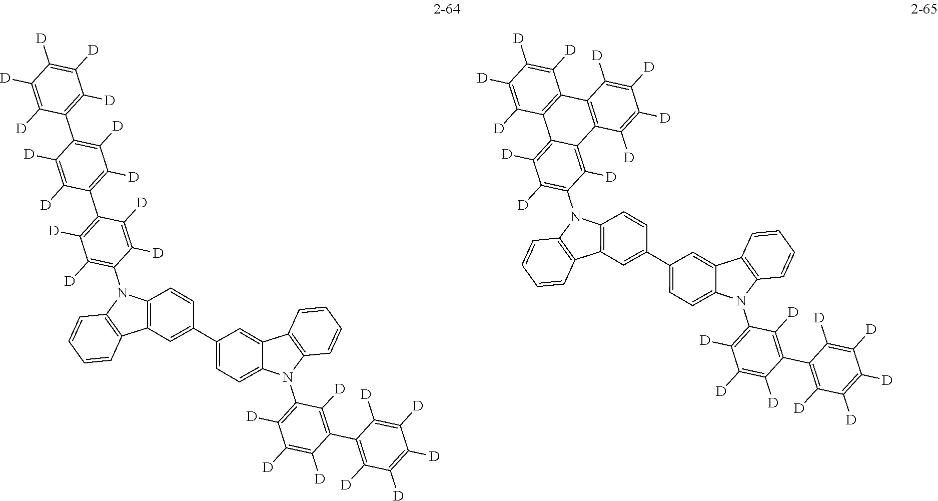 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H]]

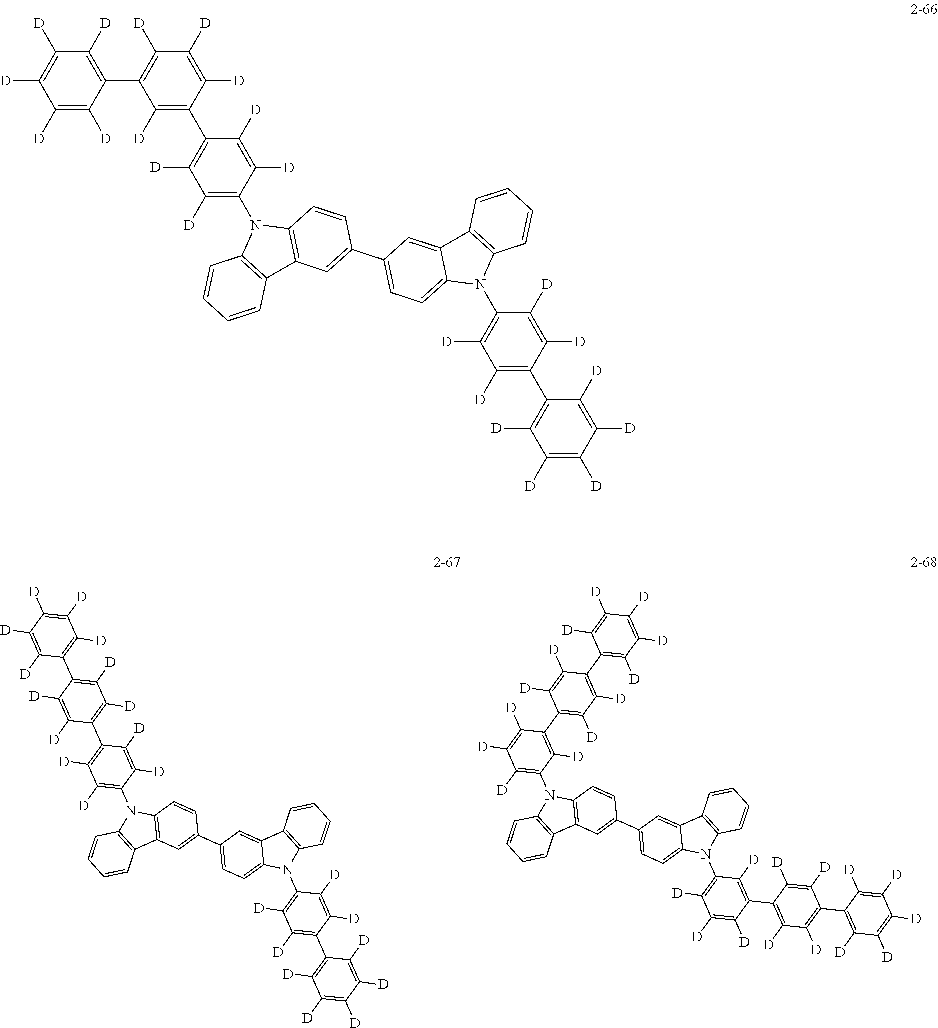

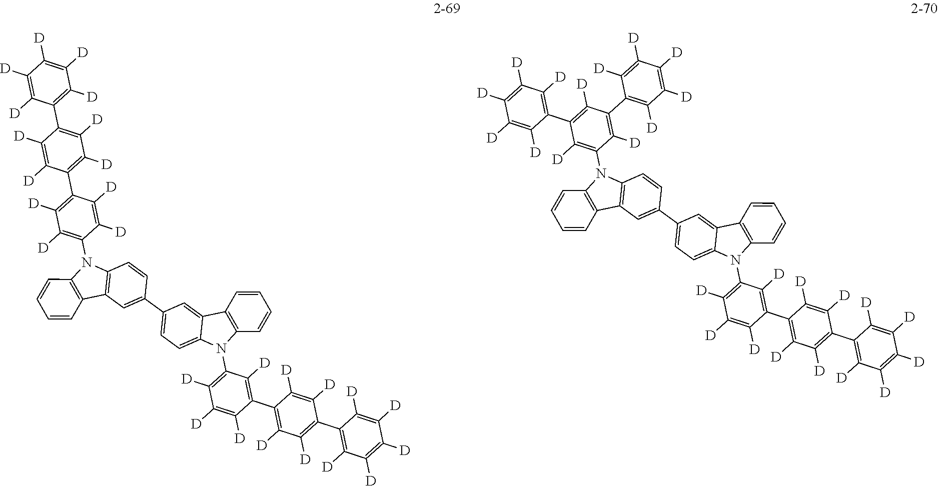

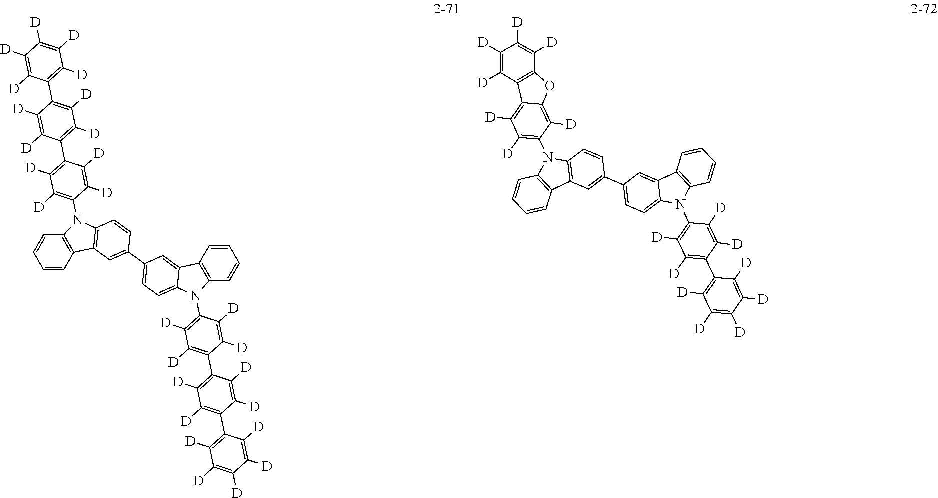 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c(-c8c([2H])c([2H])c([2H])c([2H])c8[2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]]

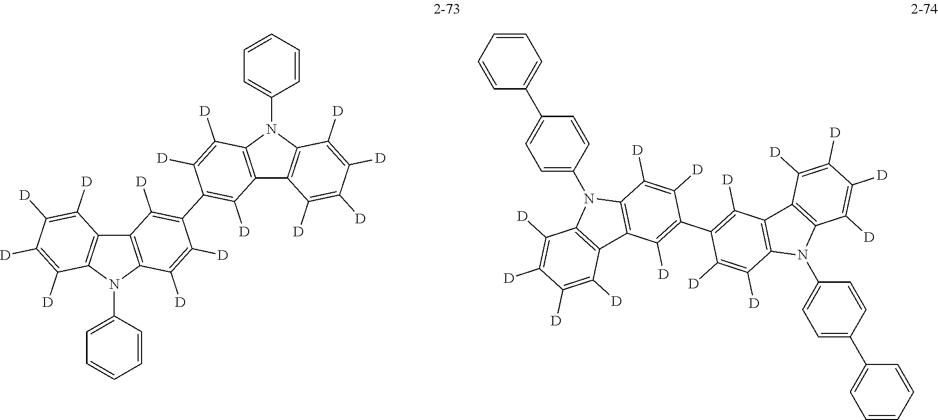 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

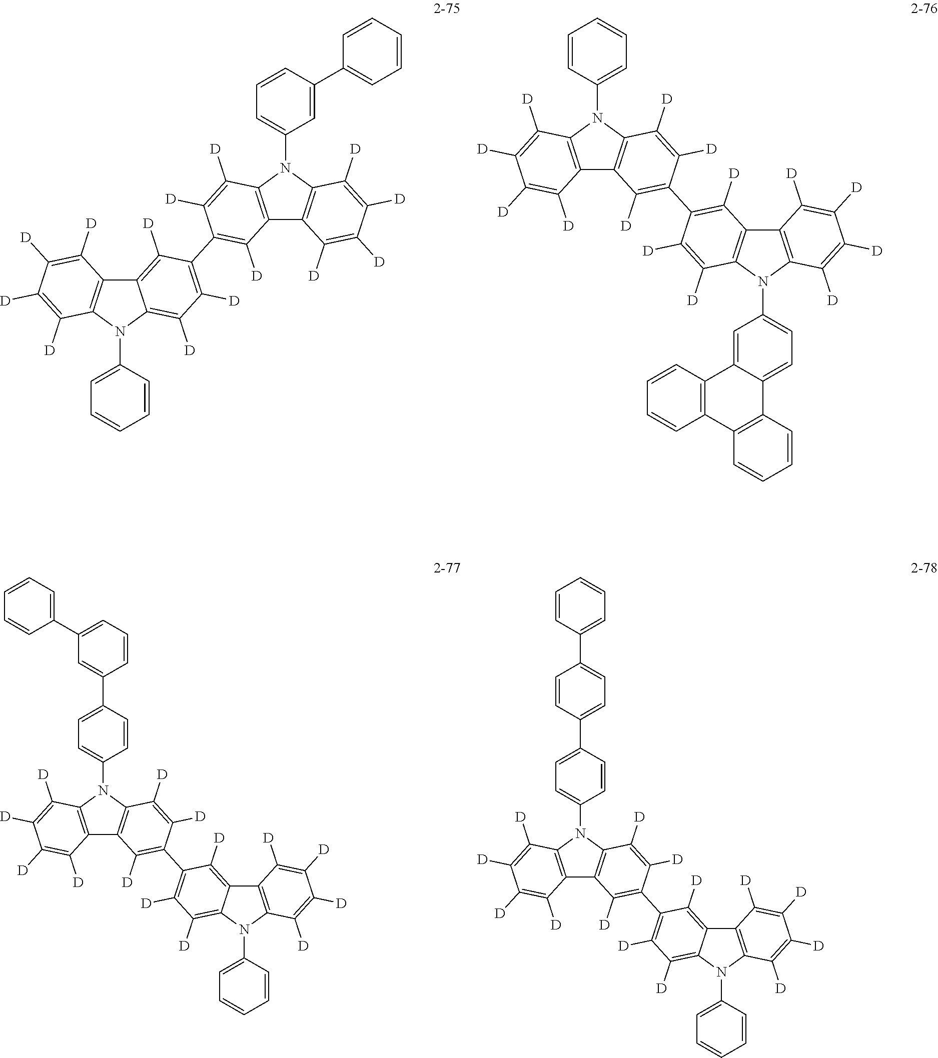

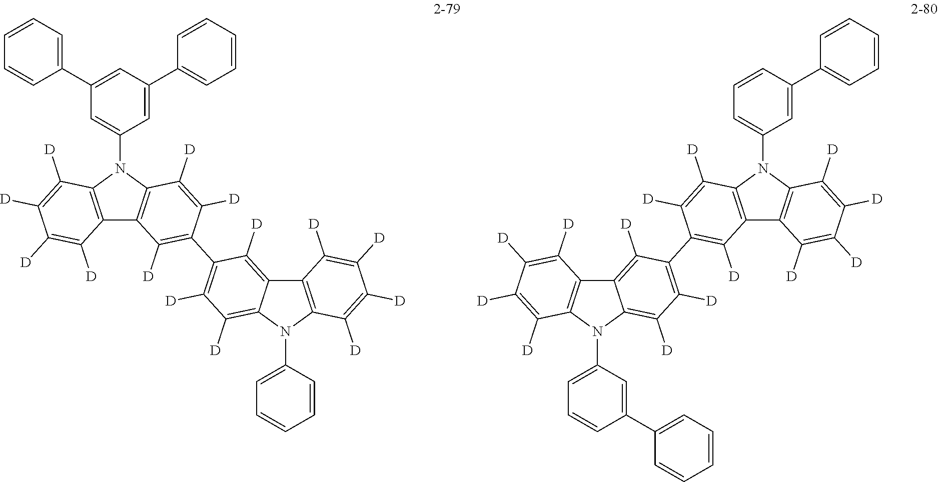 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1cccc(-c2ccccc2)c1]

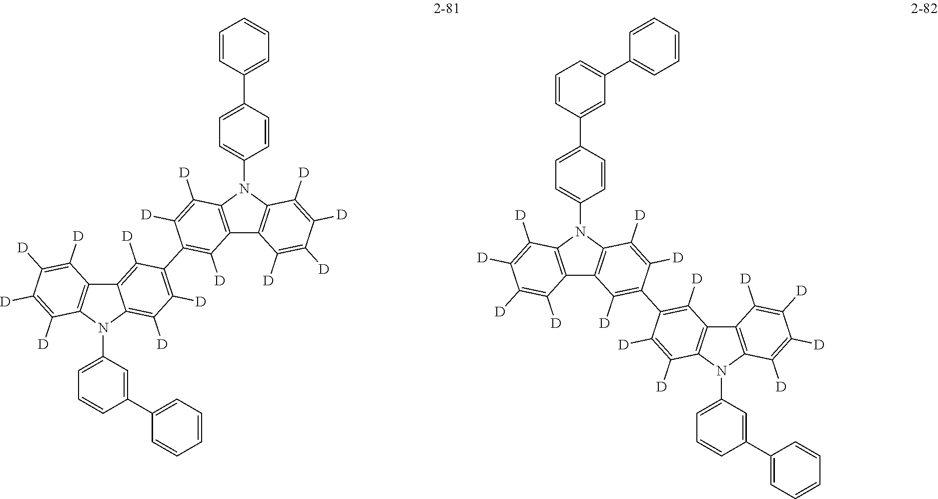 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccccc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2cccc(-c3ccccc3)c2)cc1]

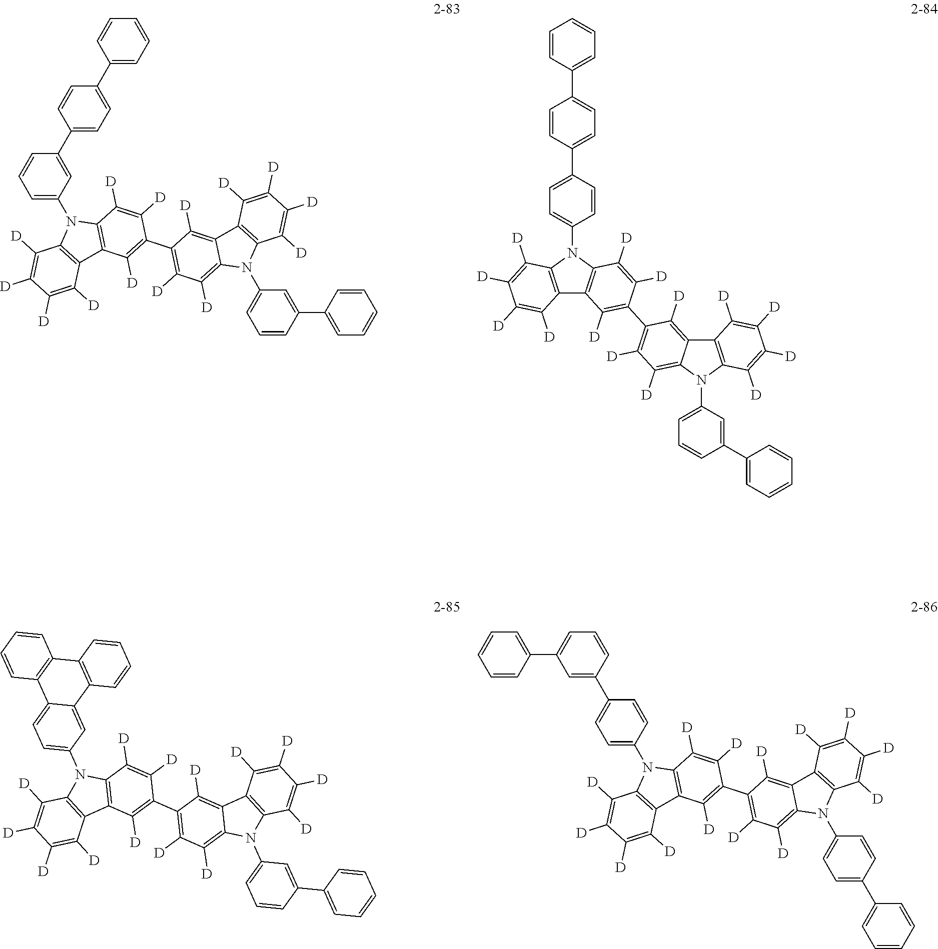

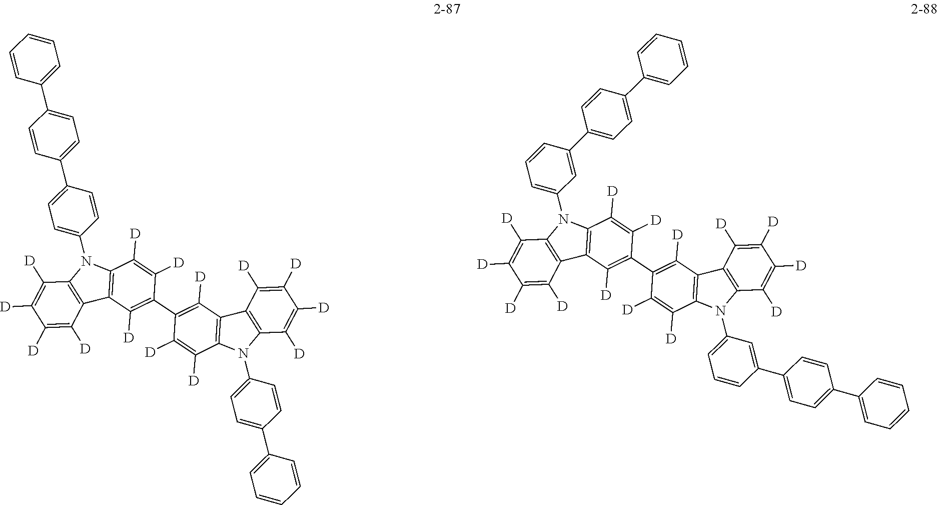 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccccc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1cccc(-c2ccc(-c3ccccc3)cc2)c1]

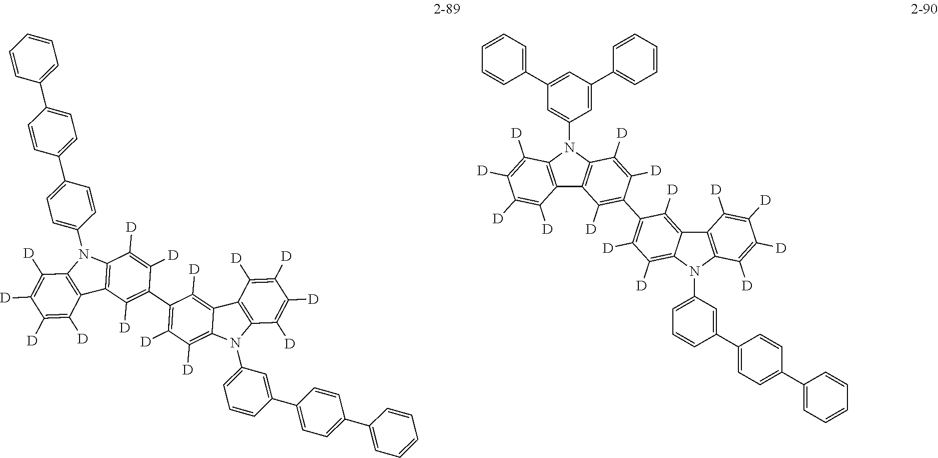 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cccc(-c2ccc(-c3ccccc3)cc2)c1]

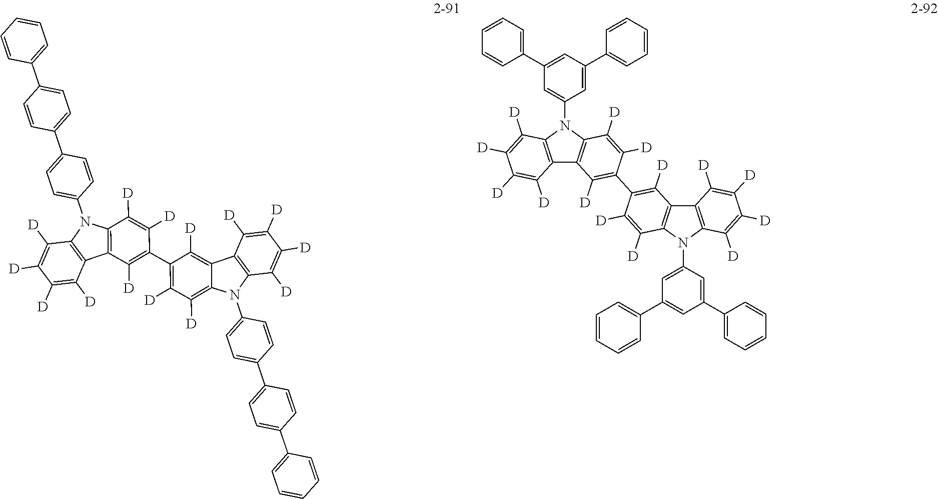 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cc(-c2ccccc2)cc(-c2ccccc2)c1]

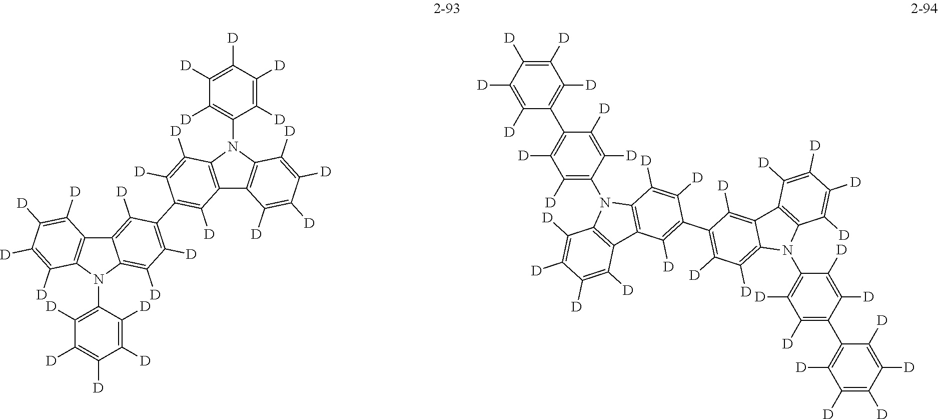 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

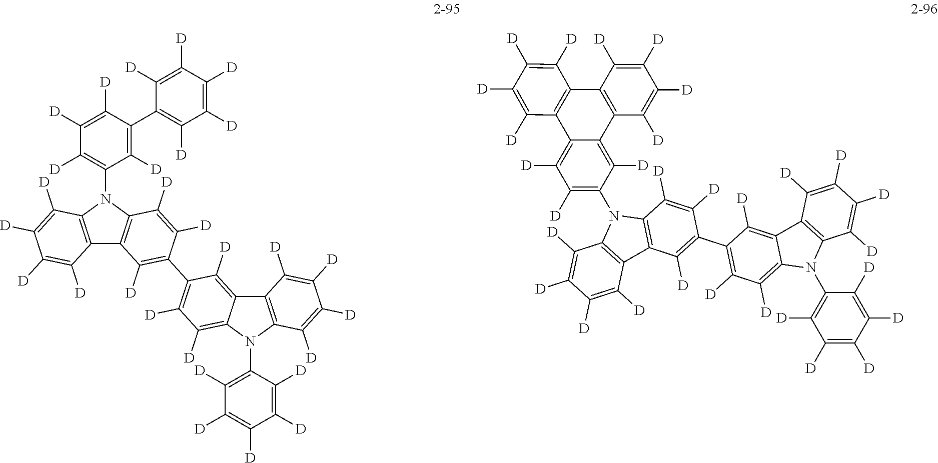 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6c6c([2H])c([2H])c([2H])c([2H])c6c5c4[2H])c([2H])c([2H])c32)c([2H])c1[2H]]

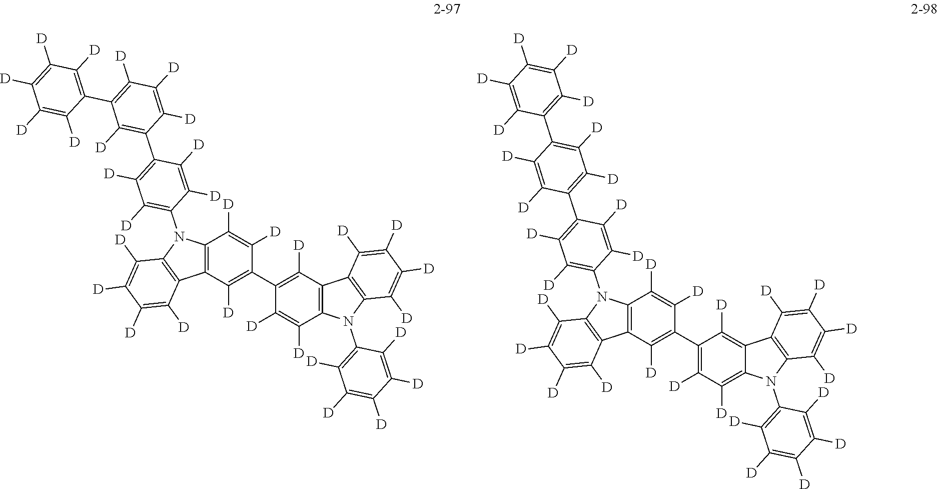

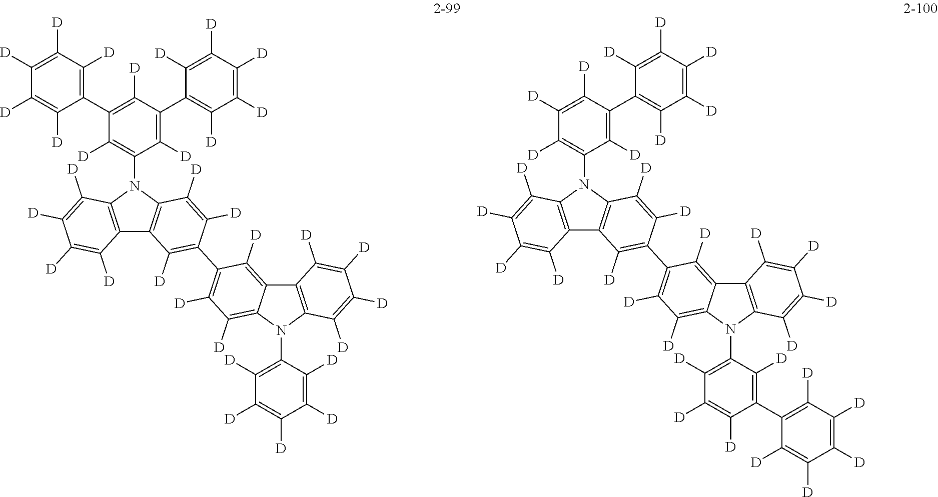

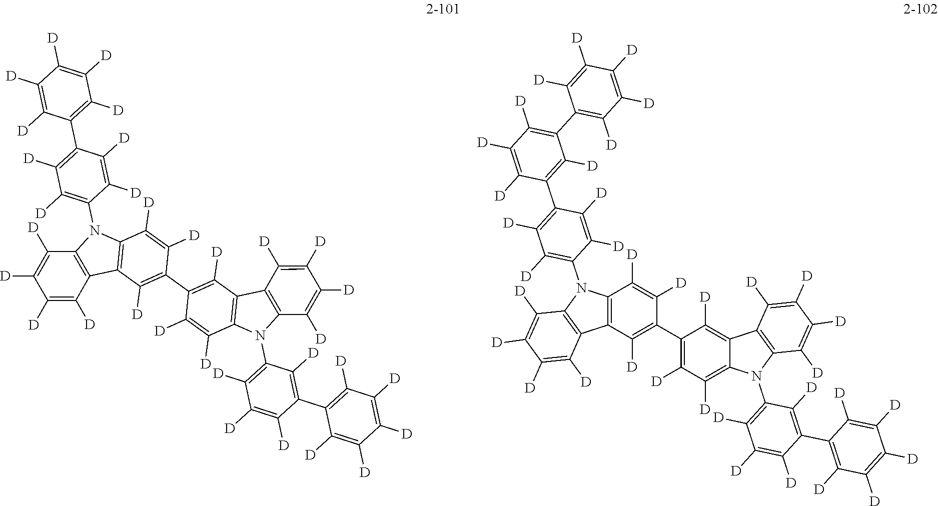

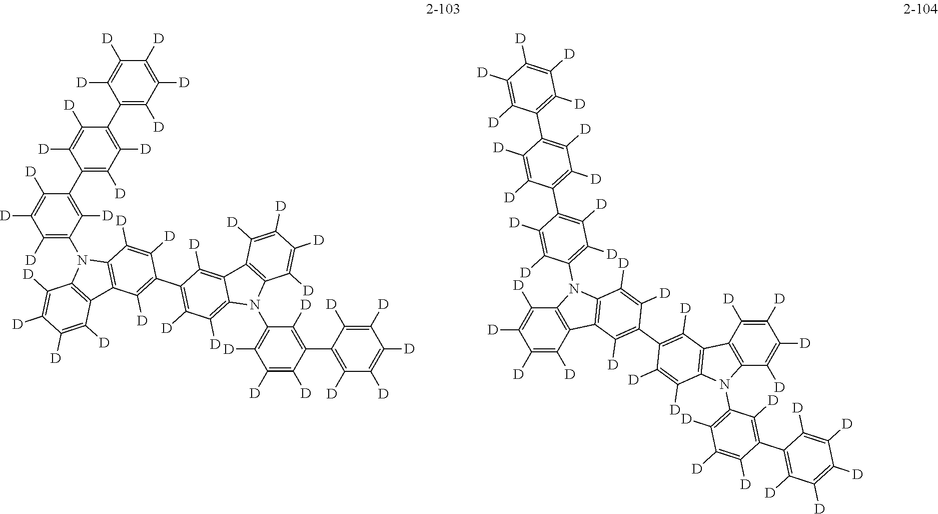

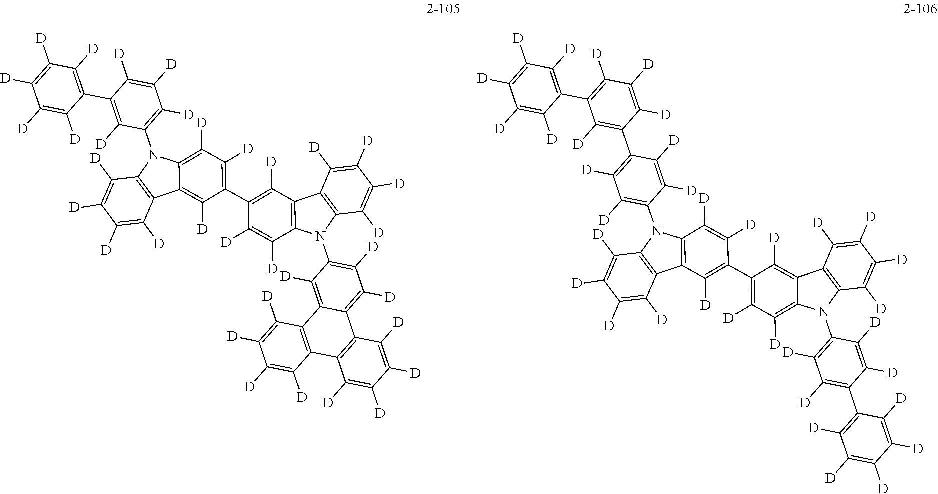

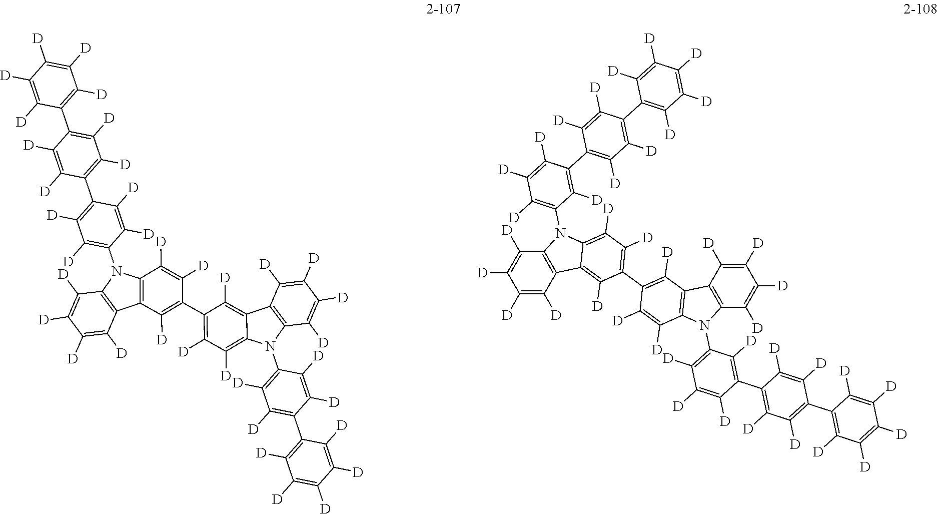

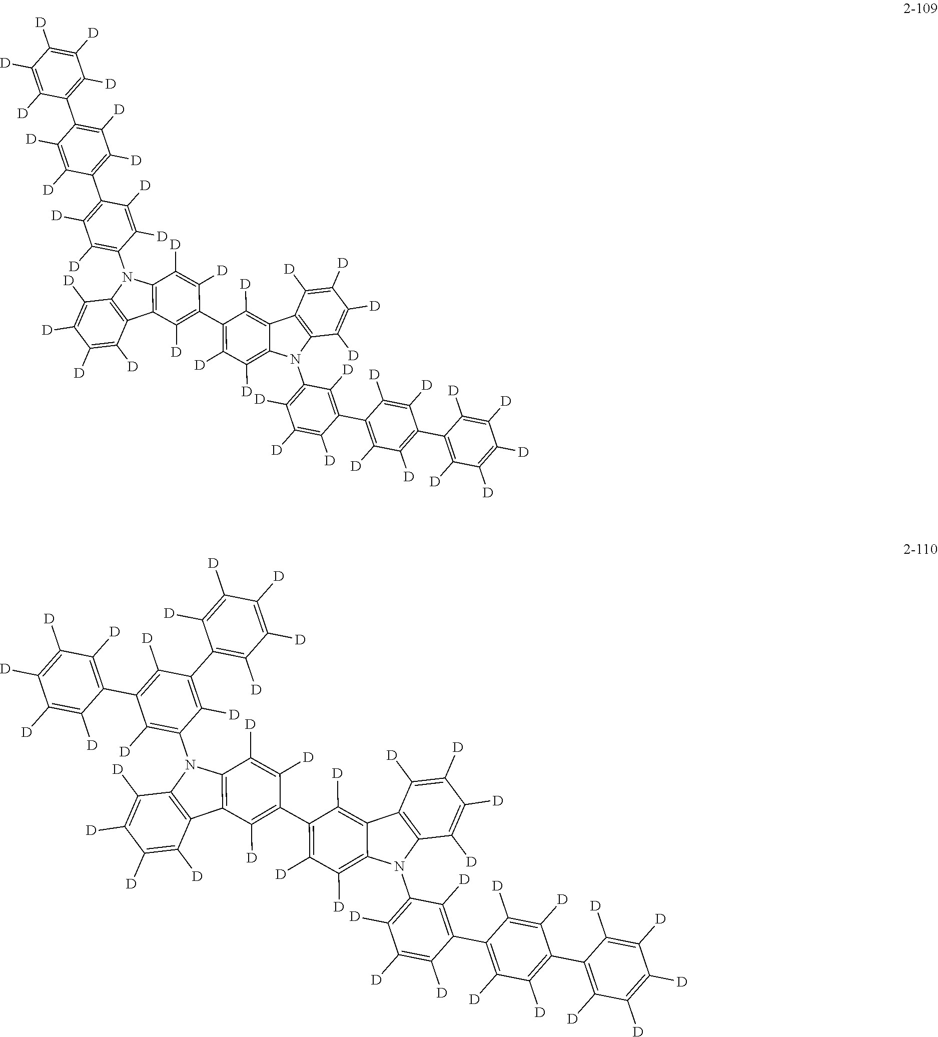

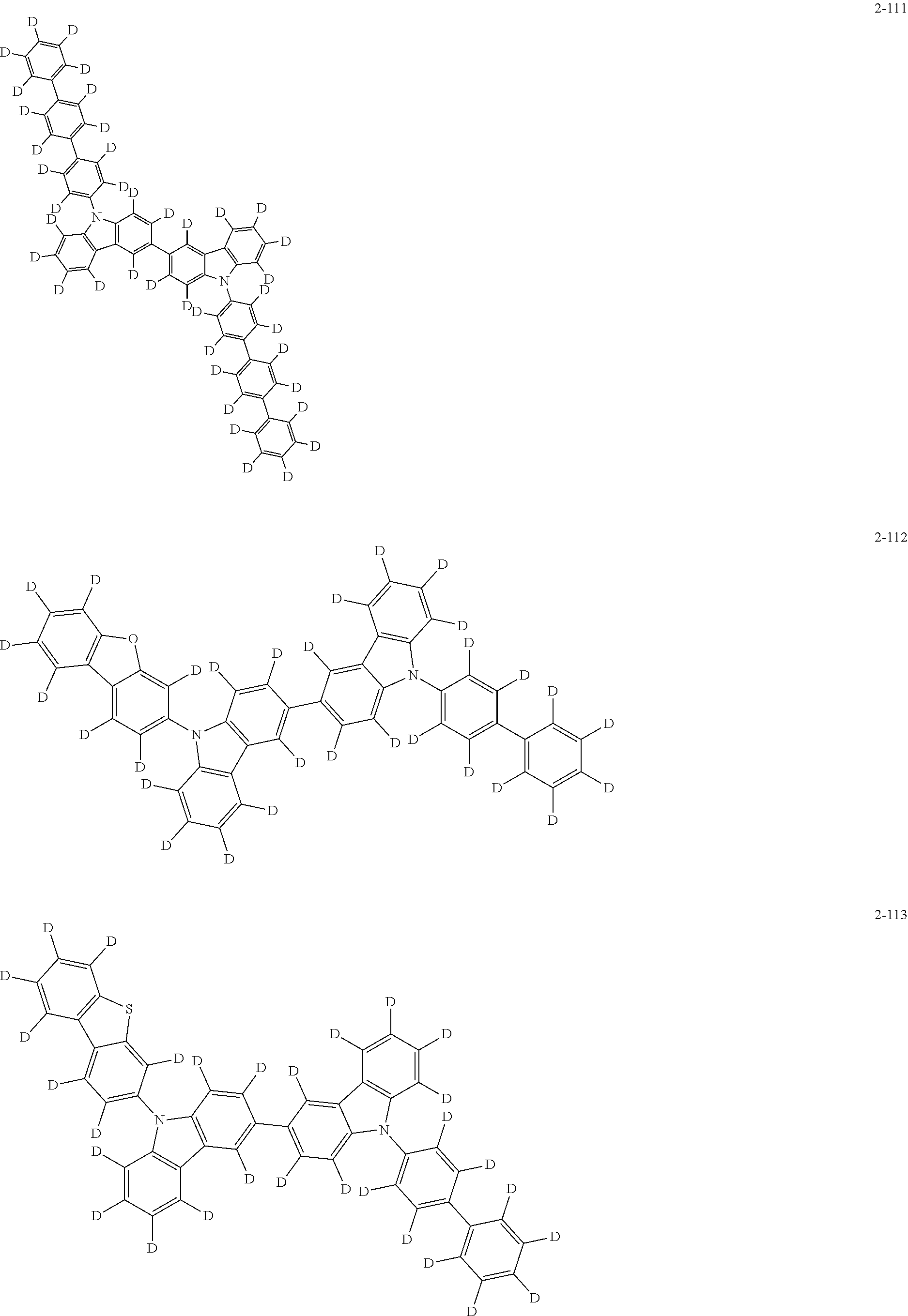

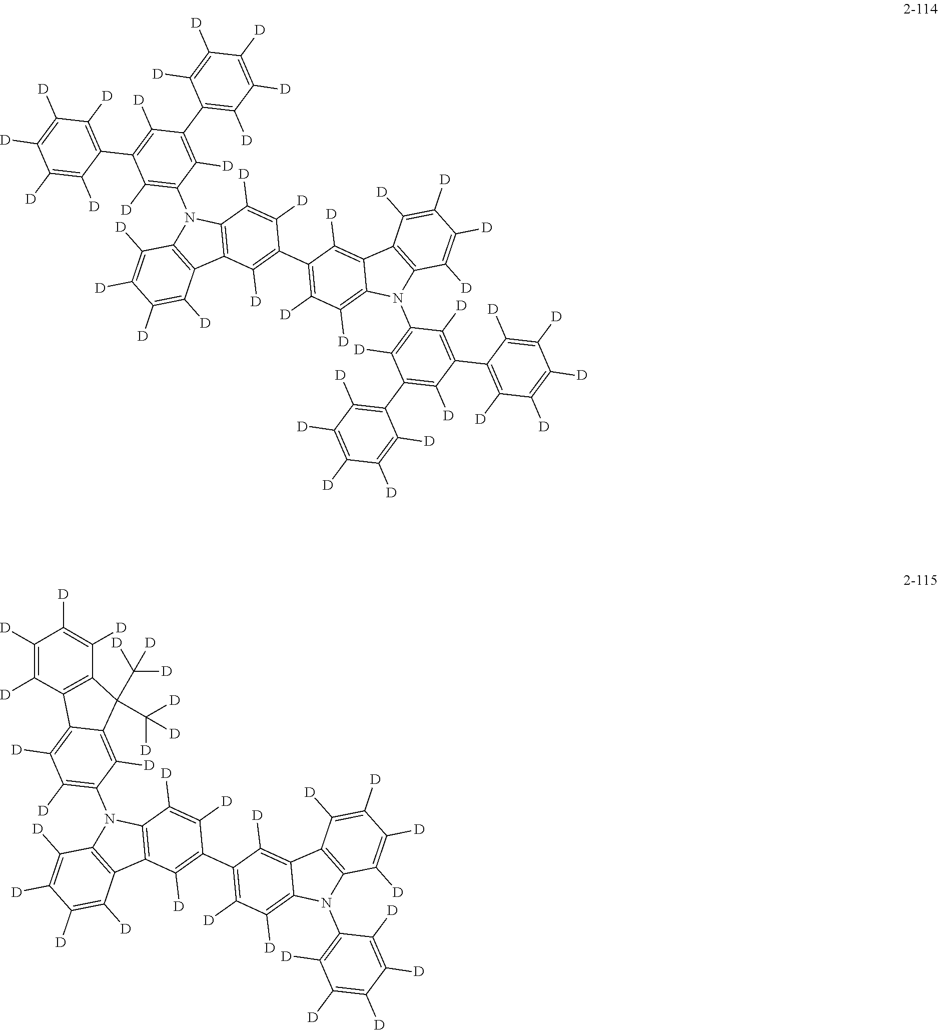

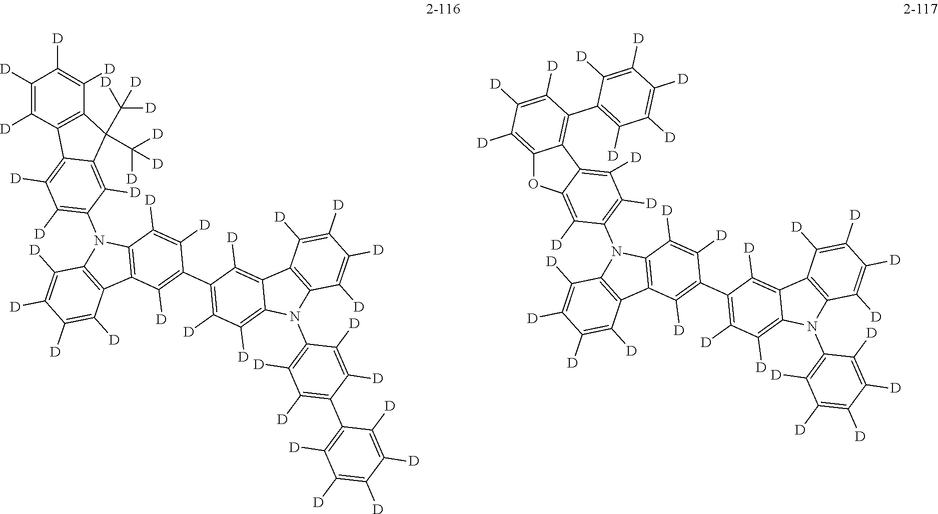

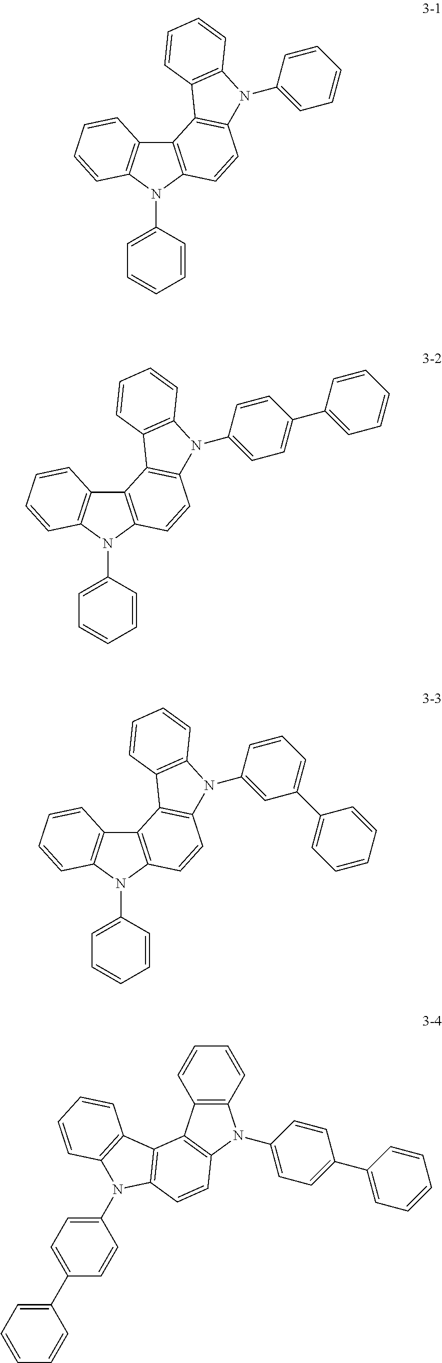 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

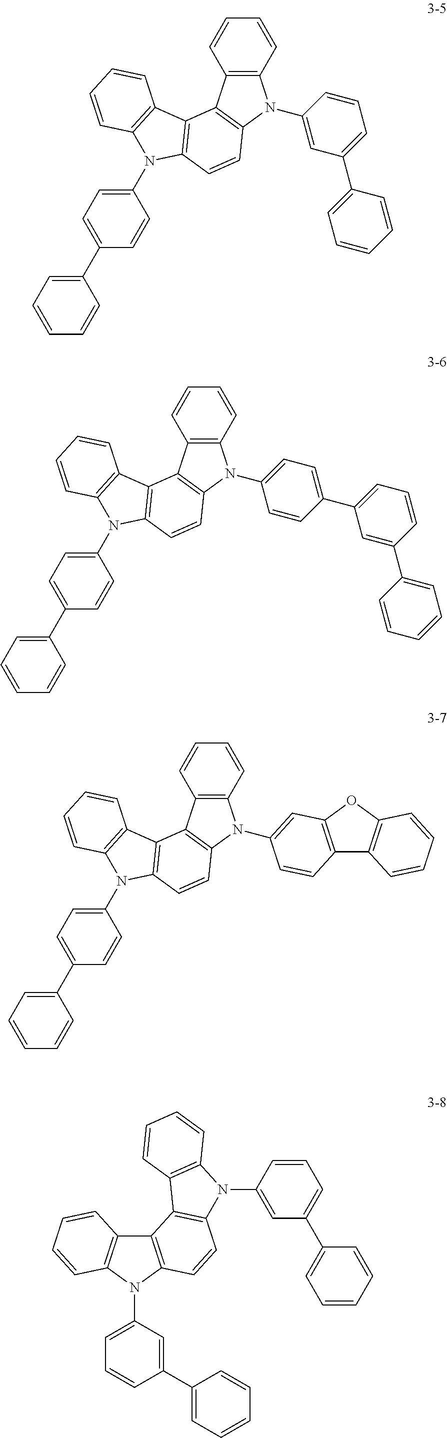 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

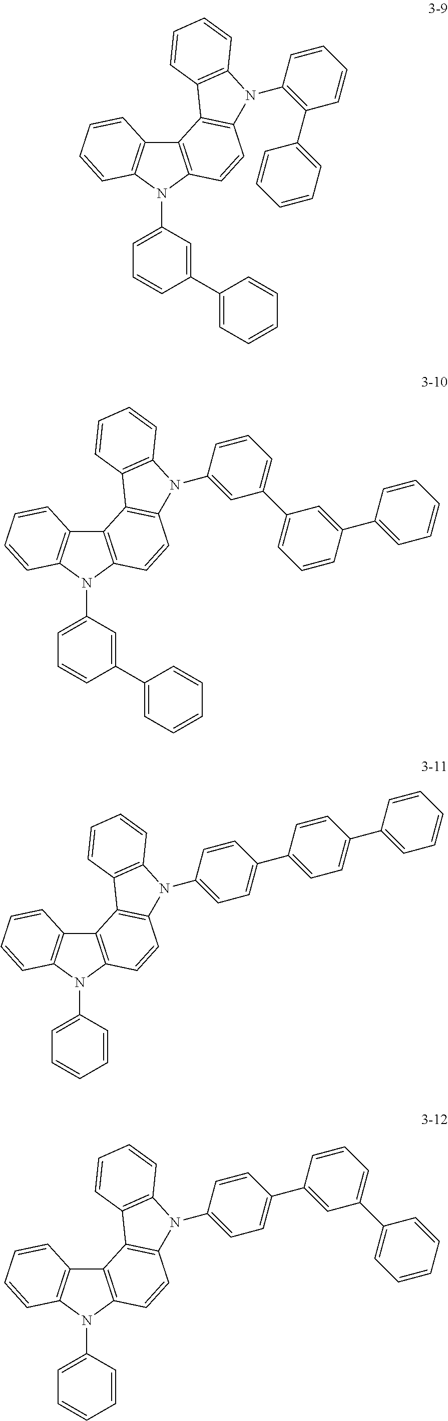 [SMILES: c1ccc(-c2cccc(-n3c4ccccc4c4c5c6ccccc6n(-c6ccccc6-c6ccccc6)c5ccc43)c2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1]

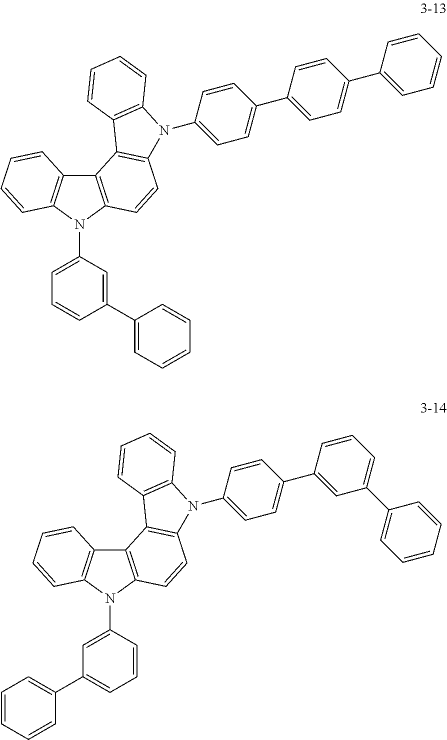 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1]

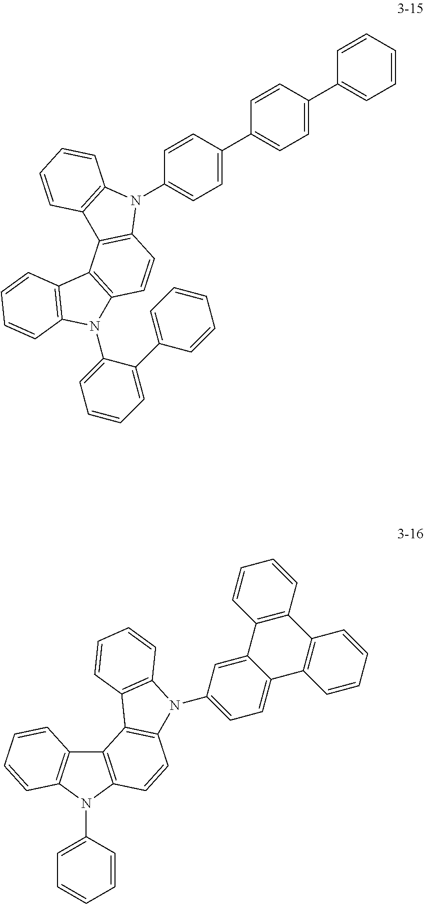 [SMILES: c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

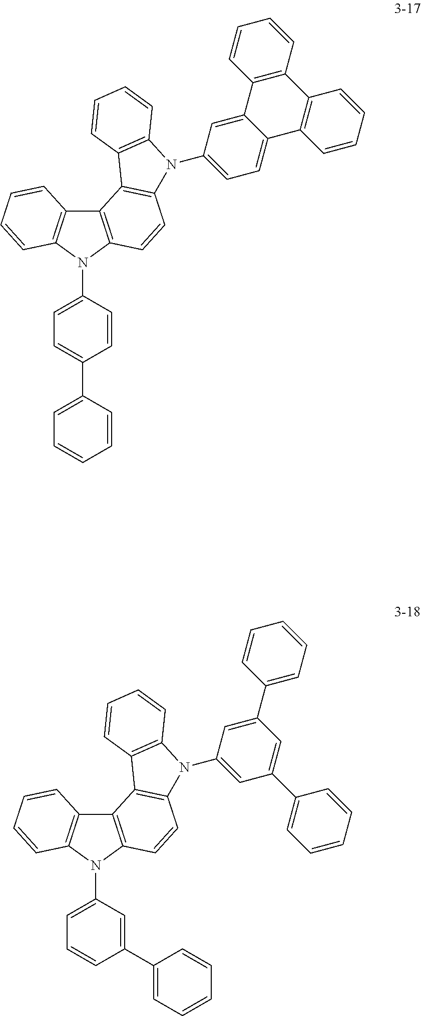 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1]

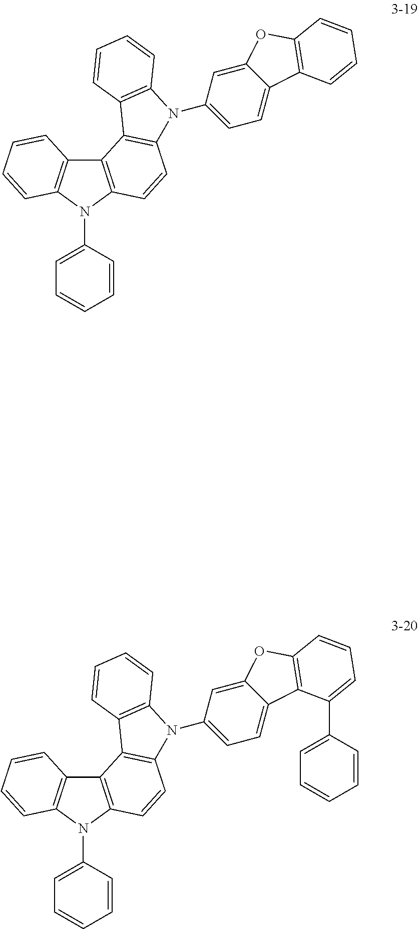 [SMILES: c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1]

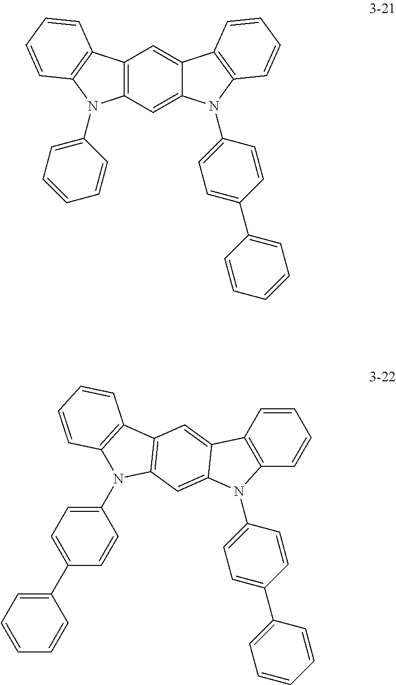 [SMILES: c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1]

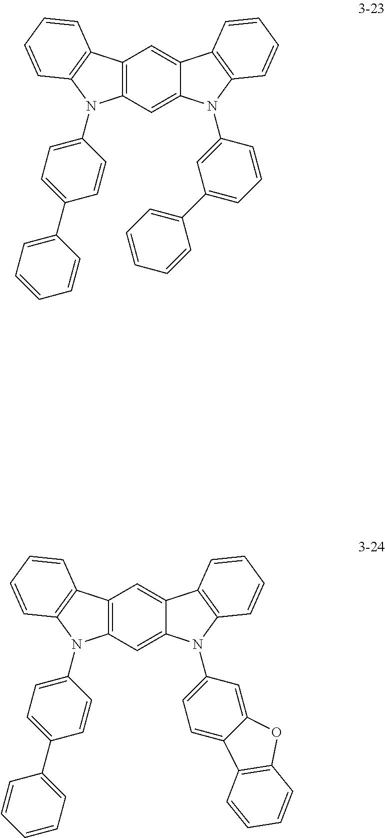 [SMILES: c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1]

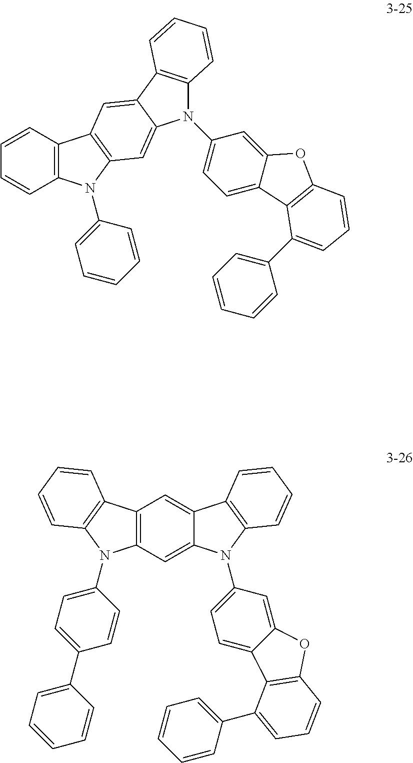 [SMILES: c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1]

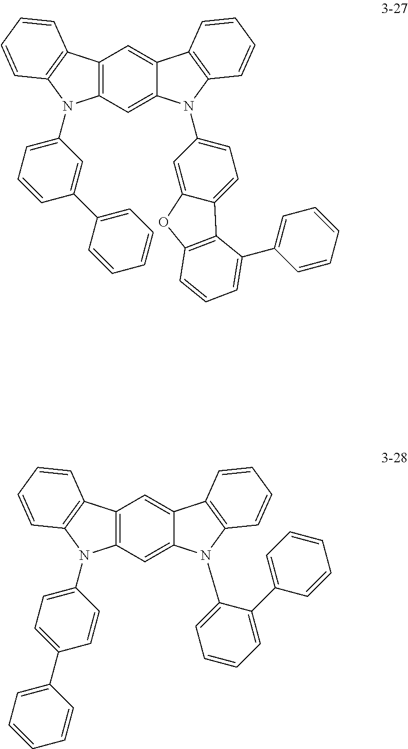 [SMILES: c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1]

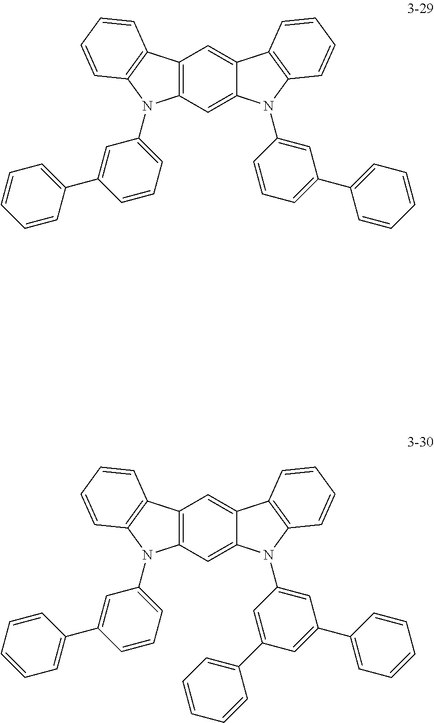 [SMILES: c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1]

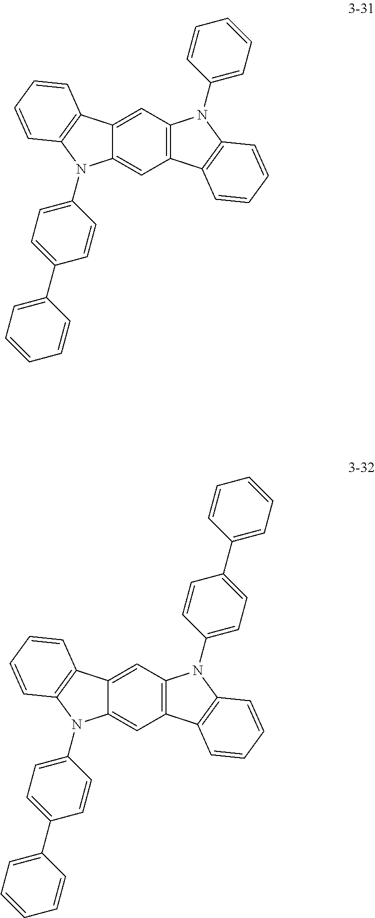 [SMILES: c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

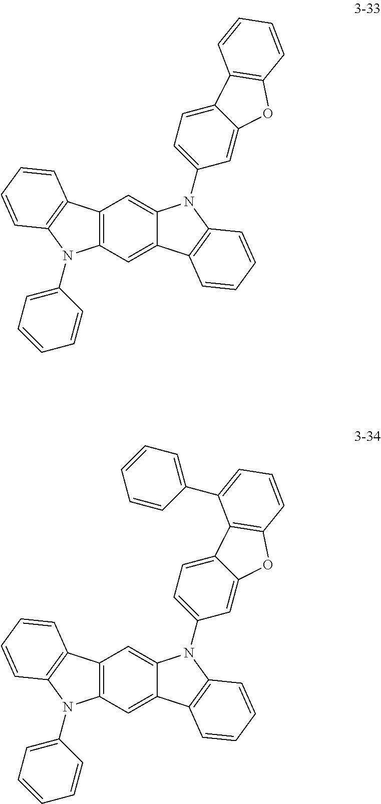 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1]

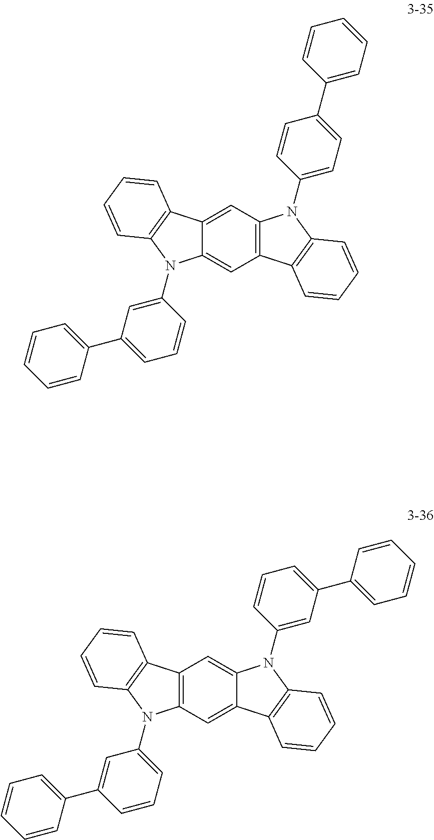 [SMILES: c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1]

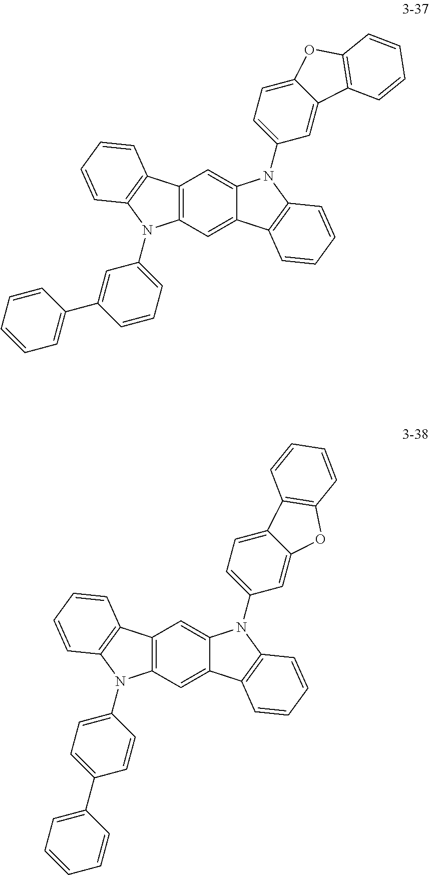 [SMILES: c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1]

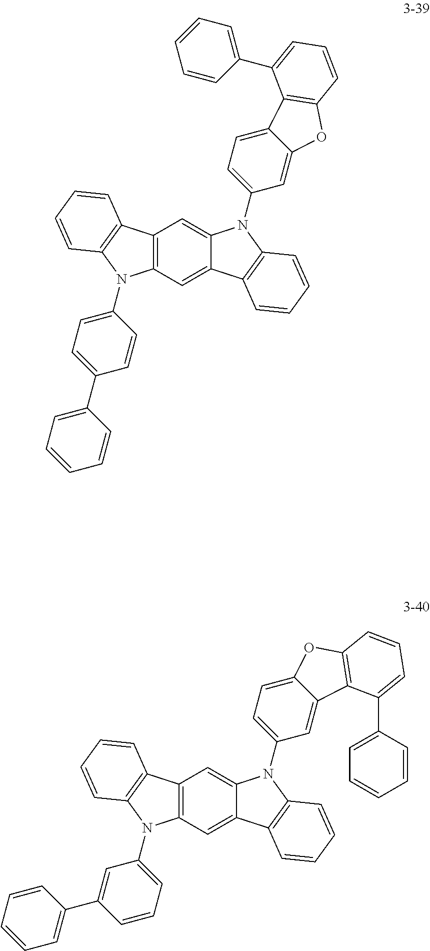 [SMILES: c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

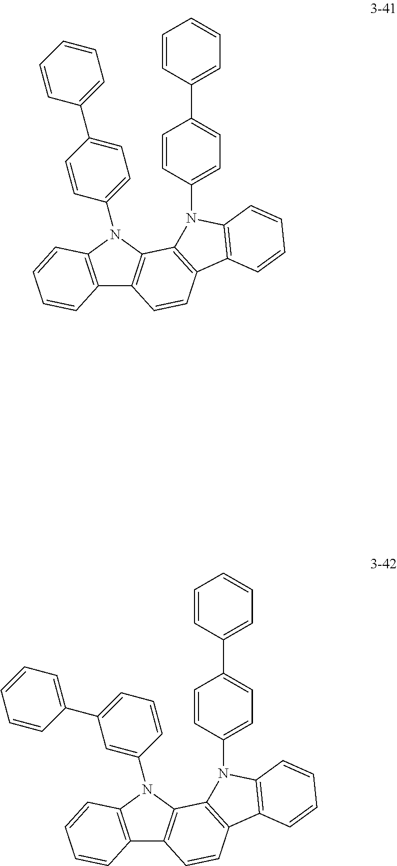 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1]

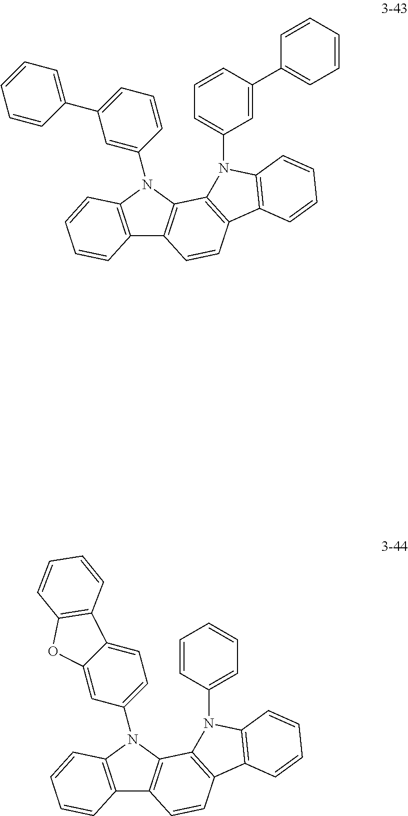 [SMILES: c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

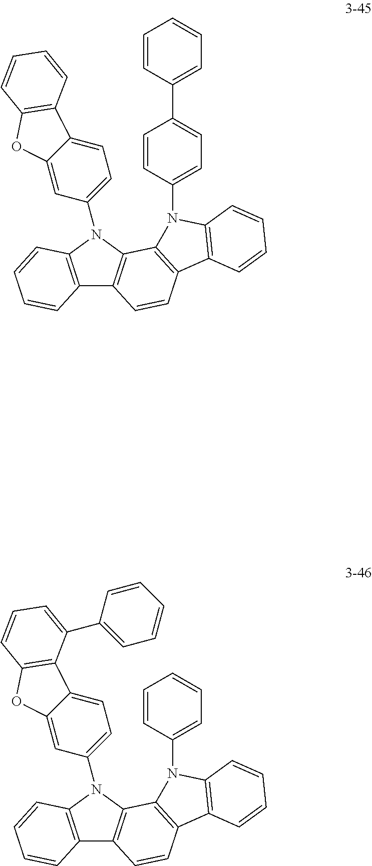 [SMILES: c1ccc(-c2ccc(-n3c4ccccc4c4ccc5c6ccccc6n(-c6ccc7c(c6)oc6ccccc67)c5c43)cc2)cc1 ; c1ccc(-c2cccc3oc4cc(-n5c6ccccc6c6ccc7c8ccccc8n(-c8ccccc8)c7c65)ccc4c23)cc1]

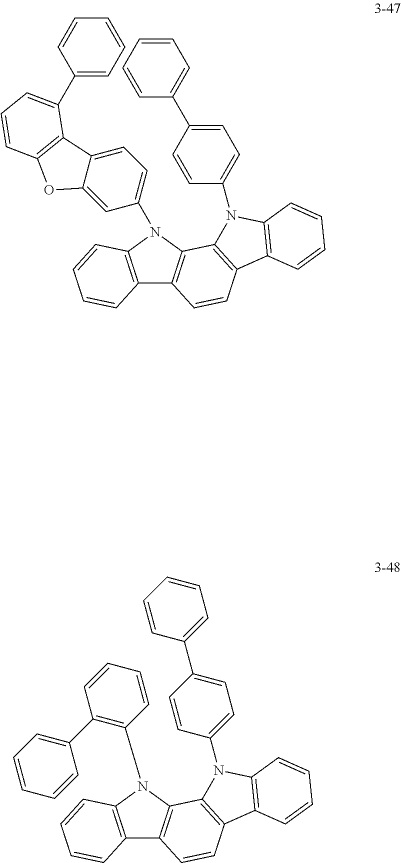 [SMILES: c1ccc(-c2ccc(-n3c4ccccc4c4ccc5c6ccccc6n(-c6ccc7c(c6)oc6cccc(-c8ccccc8)c67)c5c43)cc2)cc1 ; c1ccc(-c2ccc(-n3c4ccccc4c4ccc5c6ccccc6n(-c6ccccc6-c6ccccc6)c5c43)cc2)cc1]

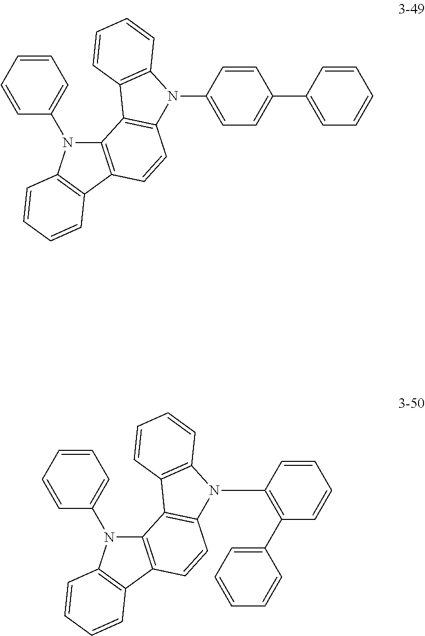 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1]

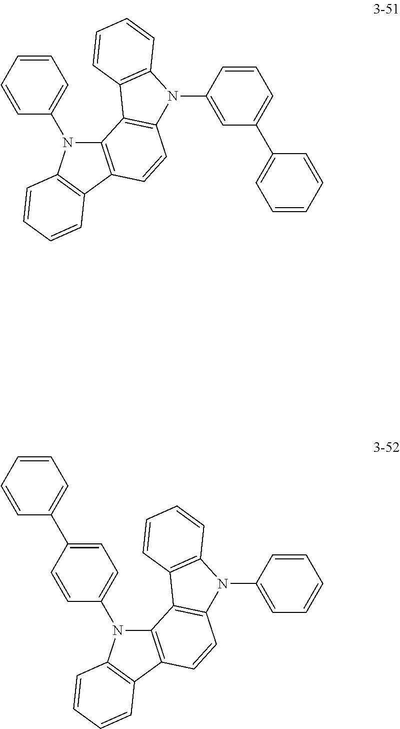 [SMILES: c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

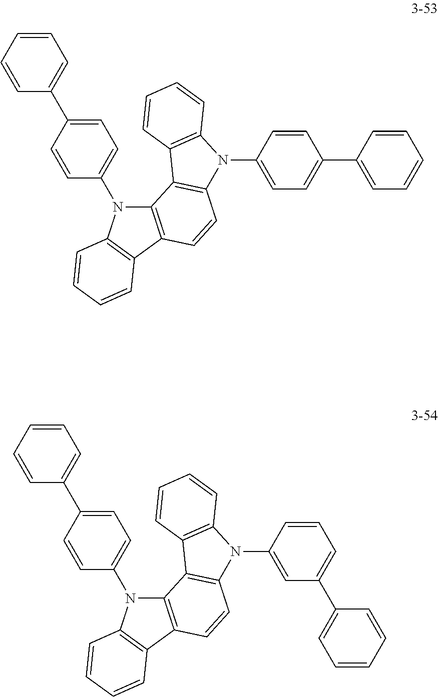 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1]

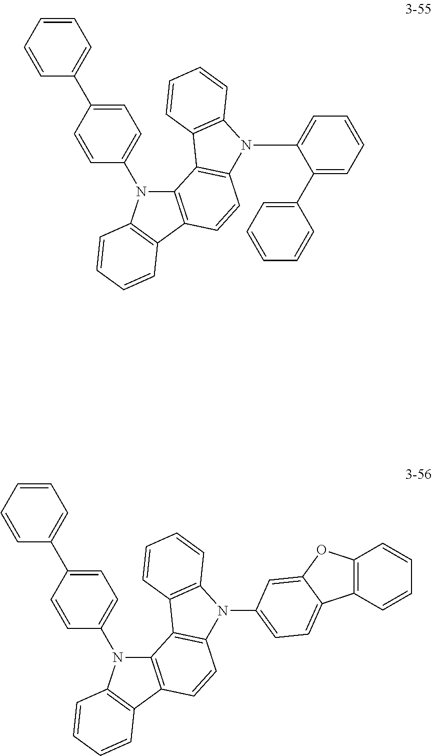 [SMILES: c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1]

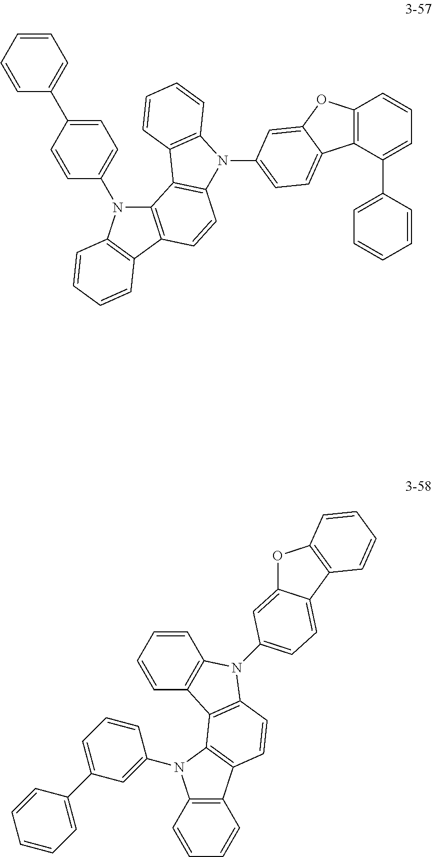 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1]

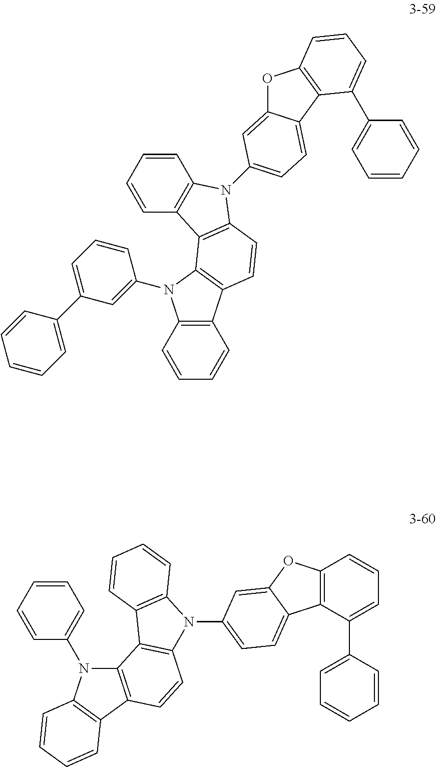 [SMILES: c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

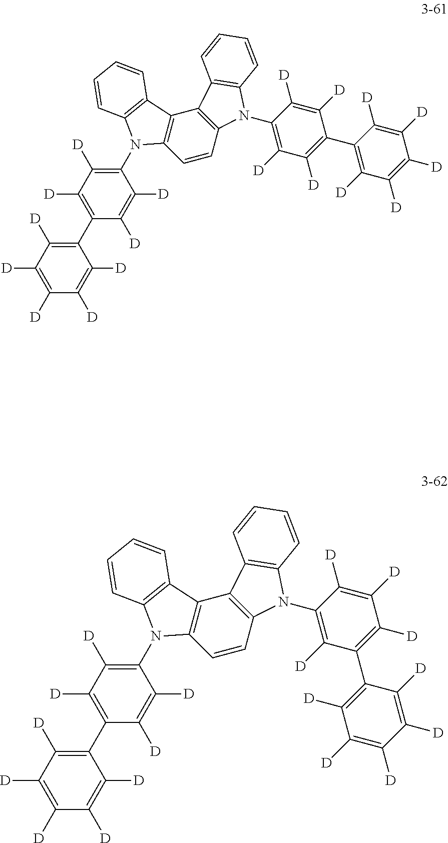 [SMILES: [2H]c1c([2H])c([2H])c(-c2c([2H])c([2H])c(-n3c4ccccc4c4c5c6ccccc6n(-c6c([2H])c([2H])c(-c7c([2H])c([2H])c([2H])c([2H])c7[2H])c([2H])c6[2H])c5ccc43)c([2H])c2[2H])c([2H])c1[2H] ; [2H]c1c([2H])c([2H])c(-c2c([2H])c([2H])c(-n3c4ccccc4c4c5c6ccccc6n(-c6c([2H])c([2H])c([2H])c(-c7c([2H])c([2H])c([2H])c([2H])c7[2H])c6[2H])c5ccc43)c([2H])c2[2H])c([2H])c1[2H]]

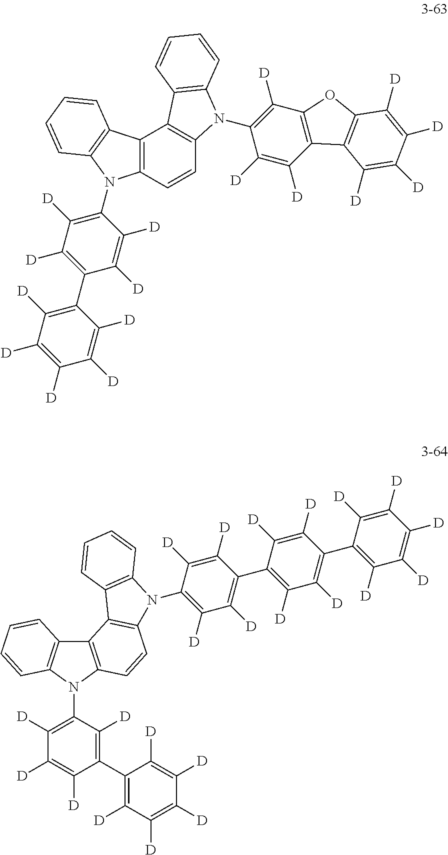 [SMILES: [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c([2H])c87)c6[2H])c5ccc43)c([2H])c2[2H])c([2H])c1[2H] ; [2H]c1c([2H])c([2H])c(-c2c([2H])c([2H])c(-c3c([2H])c([2H])c(-n4c5ccccc5c5c6c7ccccc7n(-c7c([2H])c([2H])c([2H])c(-c8c([2H])c([2H])c([2H])c([2H])c8[2H])c7[2H])c6ccc54)c([2H])c3[2H])c([2H])c2[2H])c([2H])c1[2H]]

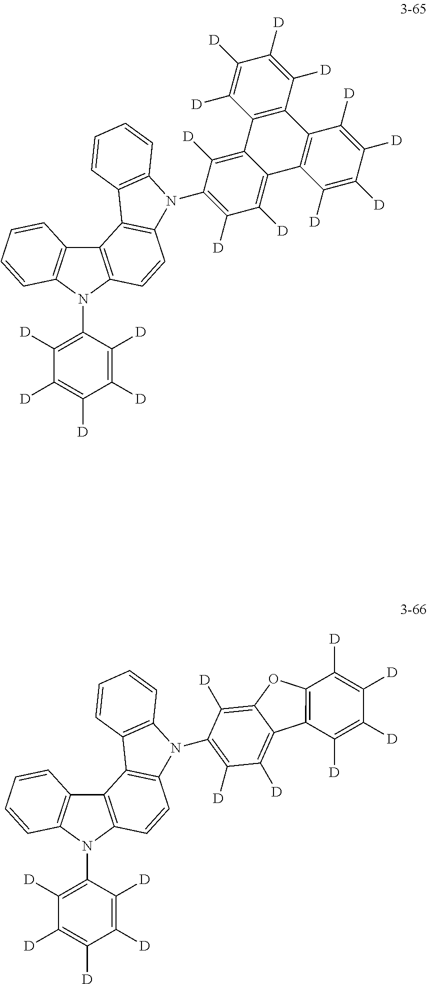 [SMILES: [2H]c1c([2H])c([2H])c(-n2c3ccccc3c3c4c5ccccc5n(-c5c([2H])c([2H])c6c7c([2H])c([2H])c([2H])c([2H])c7c7c([2H])c([2H])c([2H])c([2H])c7c6c5[2H])c4ccc32)c([2H])c1[2H] ; [2H]c1c([2H])c([2H])c(-n2c3ccccc3c3c4c5ccccc5n(-c5c([2H])c([2H])c6c(oc7c([2H])c([2H])c([2H])c([2H])c76)c5[2H])c4ccc32)c([2H])c1[2H]]

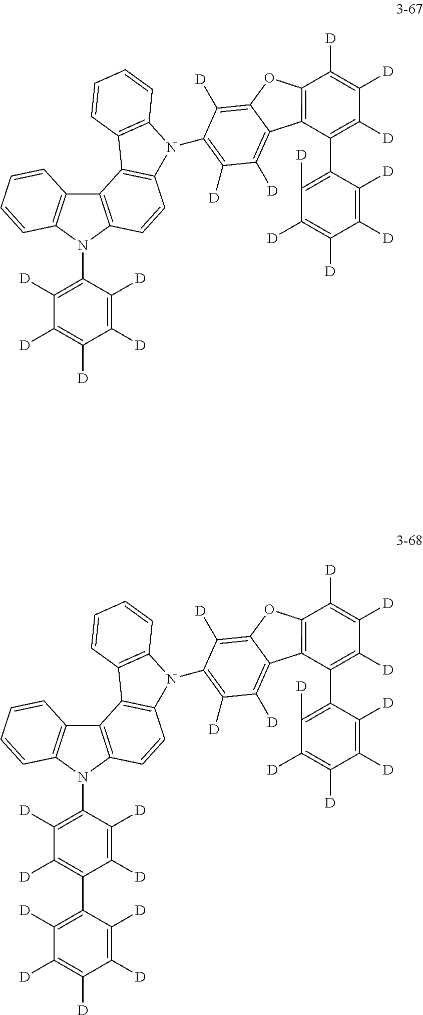 [SMILES: [2H]c1c([2H])c([2H])c(-c2c([2H])c([2H])c([2H])c3oc4c([2H])c(-n5c6ccccc6c6c7c8ccccc8n(-c8c([2H])c([2H])c([2H])c([2H])c8[2H])c7ccc65)c([2H])c([2H])c4c23)c([2H])c1[2H] ; [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c(-c9c([2H])c([2H])c([2H])c([2H])c9[2H])c87)c6[2H])c5ccc43)c([2H])c2[2H])c([2H])c1[2H]]

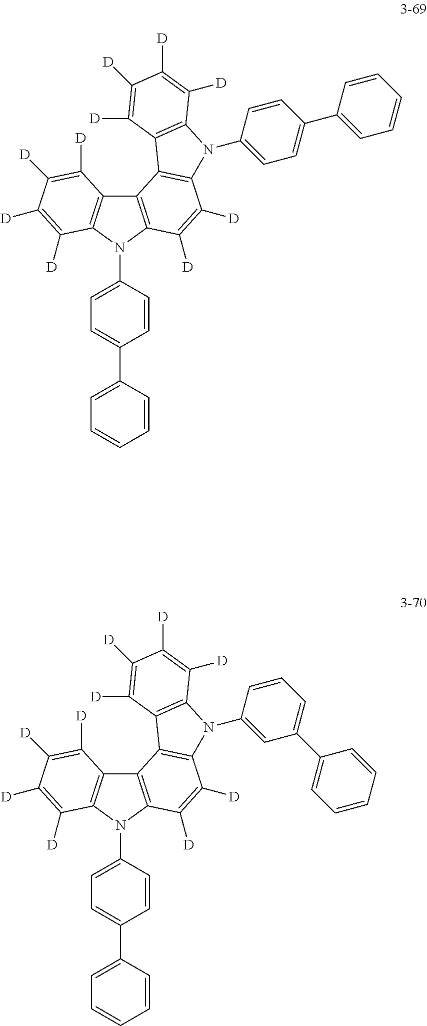 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccccc2)cc1]

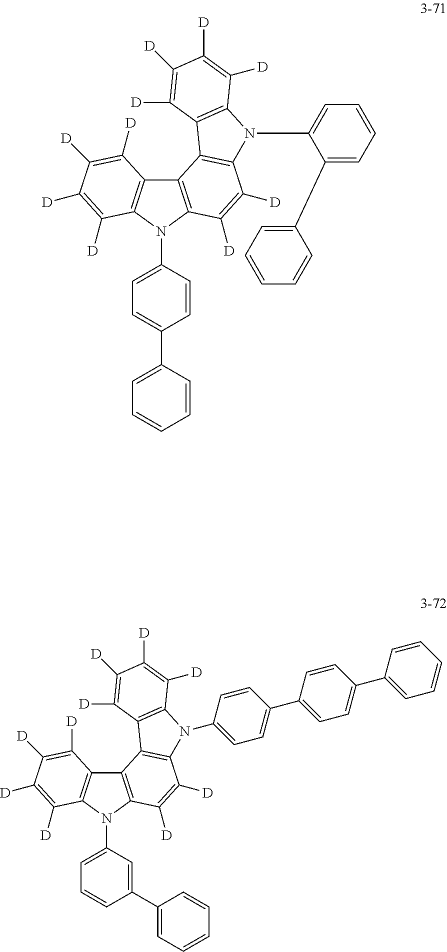 [SMILES: [2H]c1c([2H])c([2H])c2c(c1[2H])c1c3c4c([2H])c([2H])c([2H])c([2H])c4n(-c4ccccc4-c4ccccc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccc(-c3ccccc3)cc2)cc1]

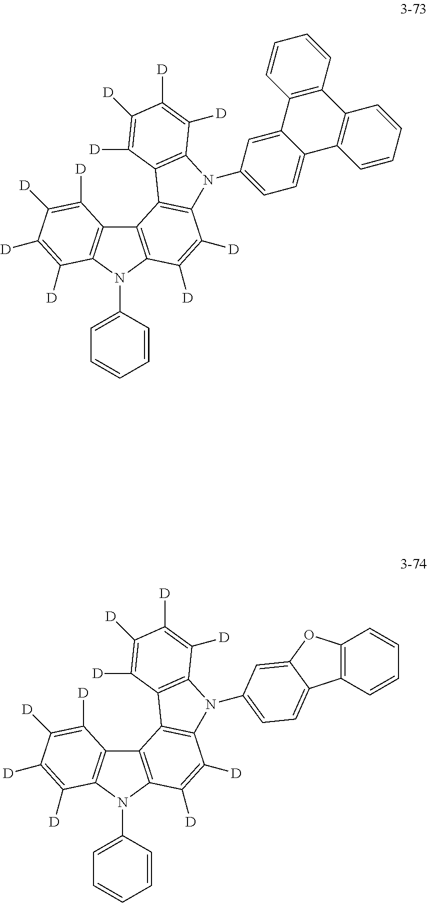 [SMILES: [2H]c1c([2H])c([2H])c2c(c1[2H])c1c3c4c([2H])c([2H])c([2H])c([2H])c4n(-c4ccc5c6ccccc6c6ccccc6c5c4)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccccc1]

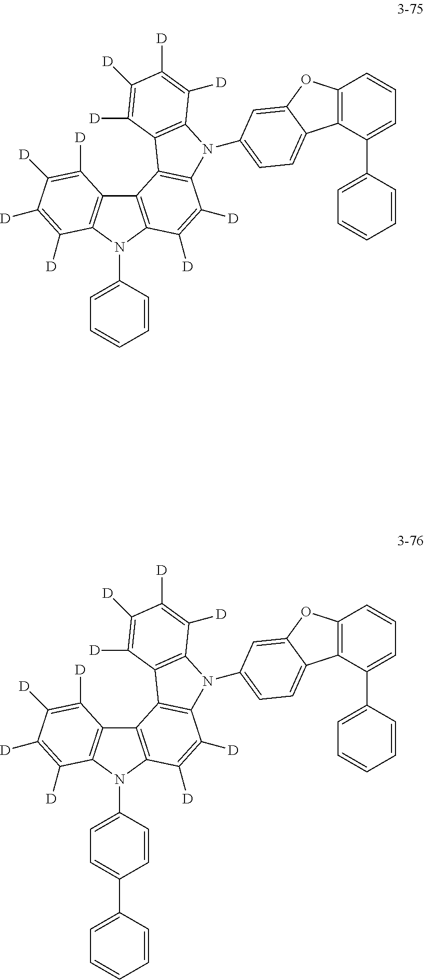 [SMILES: [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccc(-c2ccccc2)cc1]

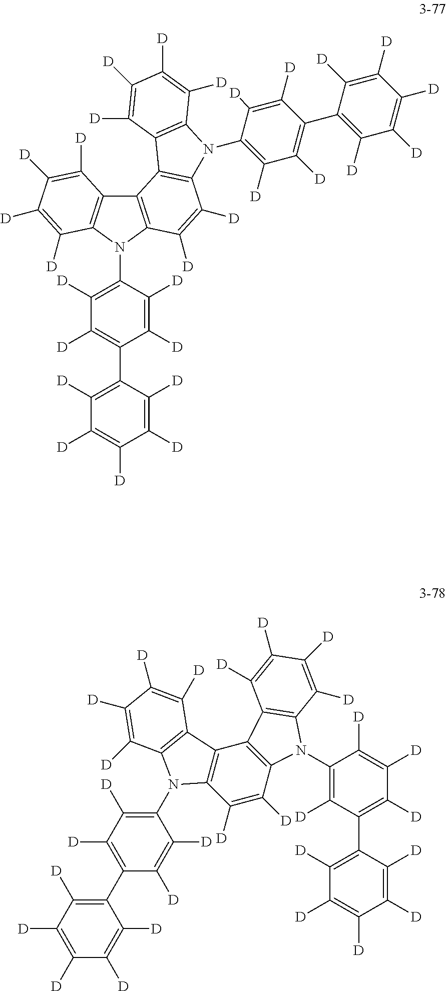 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H]]

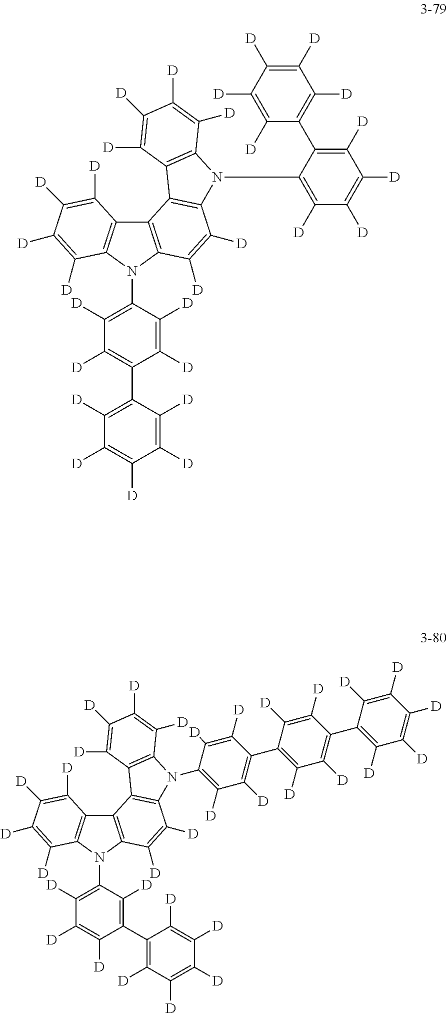 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c([2H])c6-c6c([2H])c([2H])c([2H])c([2H])c6[2H])c5c([2H])c([2H])c43)c([2H])c2[2H])c([2H])c1[2H] ; [2H]c1c([2H])c([2H])c(-c2c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c([2H])c([2H])c8[2H])c7[2H])c6c([2H])c([2H])c54)c([2H])c3[2H])c([2H])c2[2H])c([2H])c1[2H]]

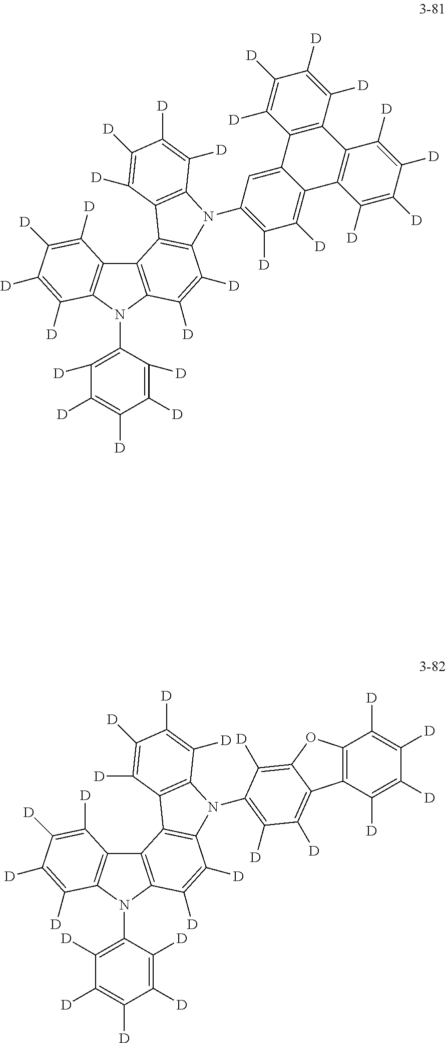 [SMILES: [2H]c1c([2H])c([2H])c(-n2c3c([2H])c([2H])c([2H])c([2H])c3c3c4c5c([2H])c([2H])c([2H])c([2H])c5n(-c5cc6c(c([2H])c5[2H])c5c([2H])c([2H])c([2H])c([2H])c5c5c([2H])c([2H])c([2H])c([2H])c65)c4c([2H])c([2H])c32)c([2H])c1[2H] ; [2H]c1c([2H])c([2H])c(-n2c3c([2H])c([2H])c([2H])c([2H])c3c3c4c5c([2H])c([2H])c([2H])c([2H])c5n(-c5c([2H])c([2H])c6c(oc7c([2H])c([2H])c([2H])c([2H])c76)c5[2H])c4c([2H])c([2H])c32)c([2H])c1[2H]]

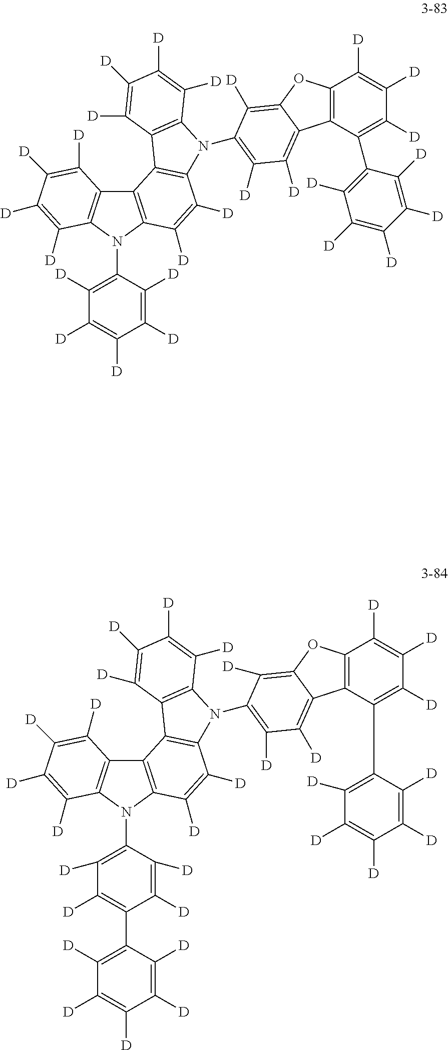 [SMILES: [2H]c1c([2H])c([2H])c(-c2c([2H])c([2H])c([2H])c3oc4c([2H])c(-n5c6c([2H])c([2H])c([2H])c([2H])c6c6c7c8c([2H])c([2H])c([2H])c([2H])c8n(-c8c([2H])c([2H])c([2H])c([2H])c8[2H])c7c([2H])c([2H])c65)c([2H])c([2H])c4c23)c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c7c(oc8c([2H])c([2H])c([2H])c(-c9c([2H])c([2H])c([2H])c([2H])c9[2H])c87)c6[2H])c5c([2H])c([2H])c43)c([2H])c2[2H])c([2H])c1[2H]]

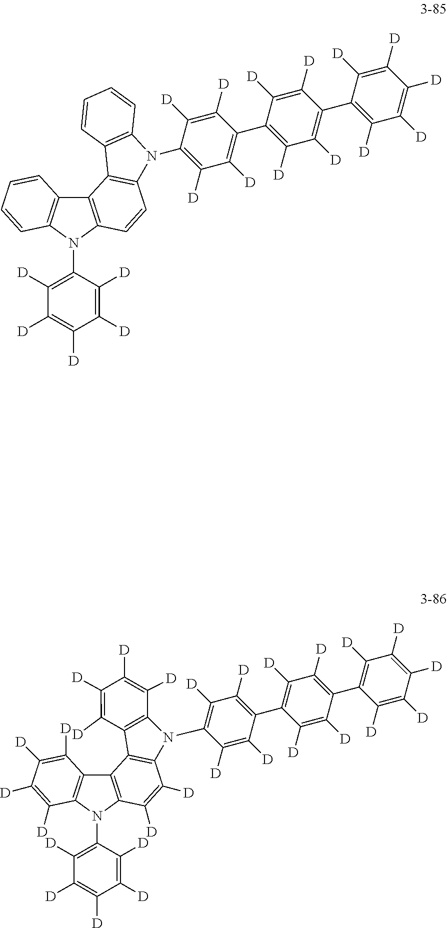 [SMILES: [2H]c1c([2H])c([2H])c(-c2c([2H])c([2H])c(-c3c([2H])c([2H])c(-n4c5ccccc5c5c6c7ccccc7n(-c7c([2H])c([2H])c([2H])c([2H])c7[2H])c6ccc54)c([2H])c3[2H])c([2H])c2[2H])c([2H])c1[2H] ; [2H]c1c([2H])c([2H])c(-c2c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c([2H])c2[2H])c([2H])c1[2H]]

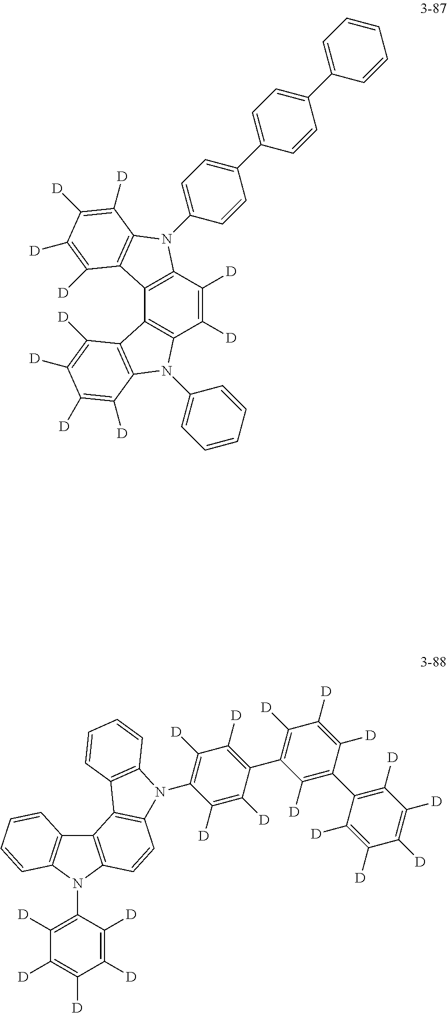 [SMILES: [2H]c1c([2H])c([2H])c2c(c1[2H])c1c3c4c([2H])c([2H])c([2H])c([2H])c4n(-c4ccc(-c5ccc(-c6ccccc6)cc5)cc4)c3c([2H])c([2H])c1n2-c1ccccc1 ; [2H]c1c([2H])c([2H])c(-c2c([2H])c([2H])c([2H])c(-c3c([2H])c([2H])c(-n4c5ccccc5c5c6c7ccccc7n(-c7c([2H])c([2H])c([2H])c([2H])c7[2H])c6ccc54)c([2H])c3[2H])c2[2H])c([2H])c1[2H]]

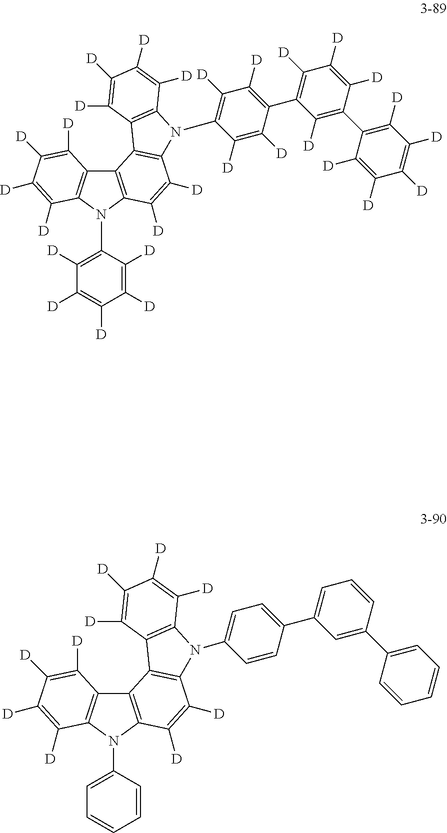 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c2[2H])c([2H])c1[2H] ; [2H]c1c([2H])c([2H])c2c(c1[2H])c1c3c4c([2H])c([2H])c([2H])c([2H])c4n(-c4ccc(-c5cccc(-c6ccccc6)c5)cc4)c3c([2H])c([2H])c1n2-c1ccccc1]

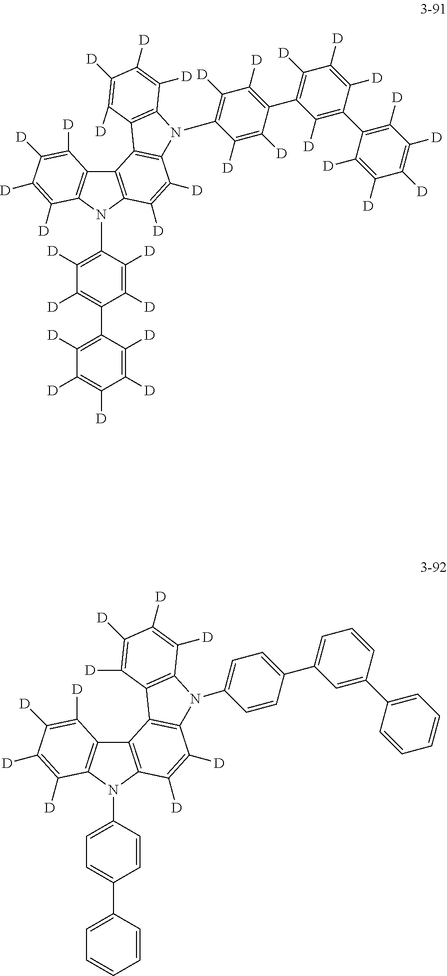 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c(-c8c([2H])c([2H])c([2H])c([2H])c8[2H])c7[2H])c([2H])c6[2H])c5c([2H])c([2H])c43)c([2H])c2[2H])c([2H])c1[2H] ; [2H]c1c([2H])c([2H])c2c(c1[2H])c1c3c4c([2H])c([2H])c([2H])c([2H])c4n(-c4ccc(-c5cccc(-c6ccccc6)c5)cc4)c3c([2H])c([2H])c1n2-c1ccc(-c2ccccc2)cc1]

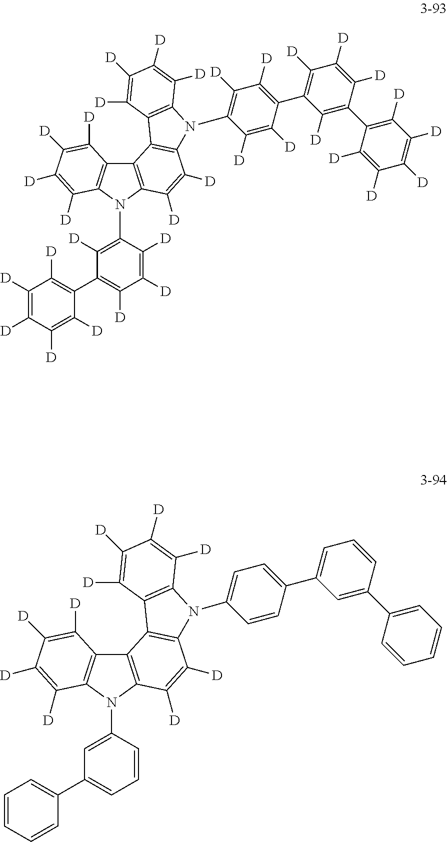 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c([2H])c([2H])c8[2H])c7[2H])c6c([2H])c([2H])c54)c([2H])c3[2H])c2[2H])c([2H])c1[2H] ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2cccc(-c3ccccc3)c2)cc1]

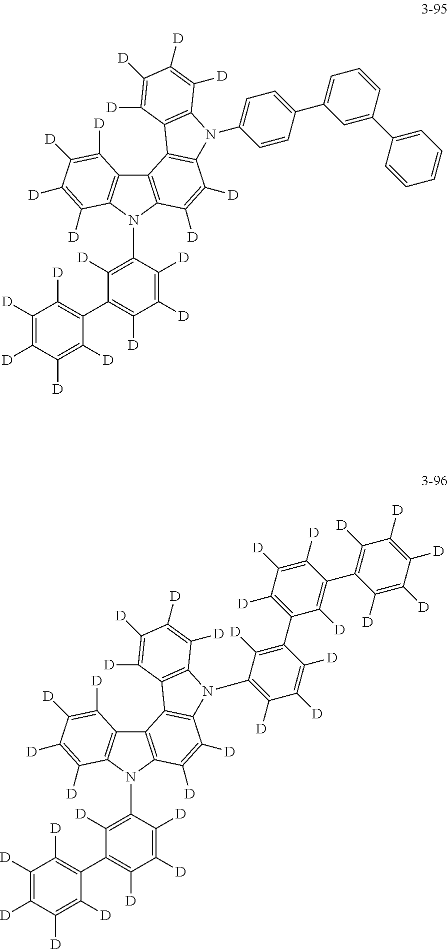 [SMILES: [2H]c1c([2H])c([2H])c(-c2c([2H])c([2H])c([2H])c(-n3c4c([2H])c([2H])c([2H])c([2H])c4c4c5c6c([2H])c([2H])c([2H])c([2H])c6n(-c6ccc(-c7cccc(-c8ccccc8)c7)cc6)c5c([2H])c([2H])c43)c2[2H])c([2H])c1[2H] ; [2H]c1c([2H])c([2H])c(-c2c([2H])c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c([2H])c([2H])c8[2H])c7[2H])c6c([2H])c([2H])c54)c3[2H])c2[2H])c([2H])c1[2H]]

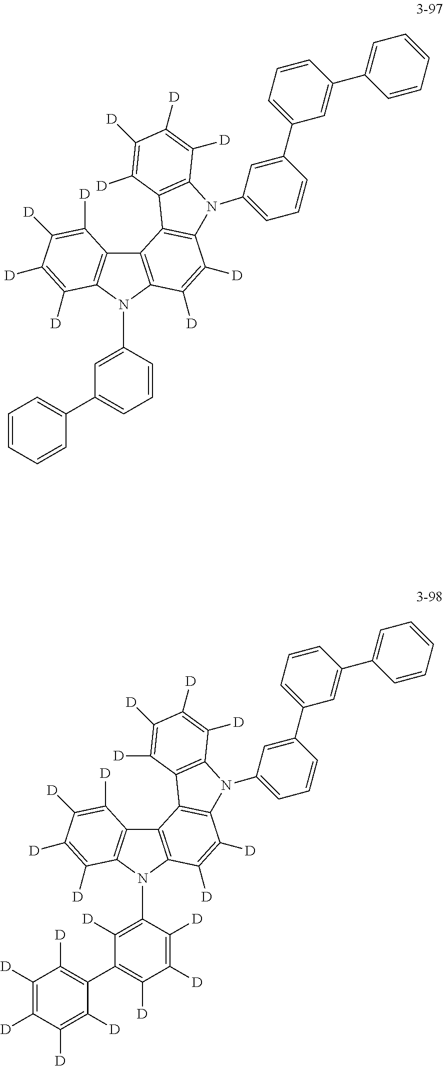 [SMILES: [2H]c1c([2H])c([2H])c2c(c1[2H])c1c3c4c([2H])c([2H])c([2H])c([2H])c4n(-c4cccc(-c5cccc(-c6ccccc6)c5)c4)c3c([2H])c([2H])c1n2-c1cccc(-c2ccccc2)c1 ; [2H]c1c([2H])c([2H])c(-c2c([2H])c([2H])c([2H])c(-n3c4c([2H])c([2H])c([2H])c([2H])c4c4c5c6c([2H])c([2H])c([2H])c([2H])c6n(-c6cccc(-c7cccc(-c8ccccc8)c7)c6)c5c([2H])c([2H])c43)c2[2H])c([2H])c1[2H]]

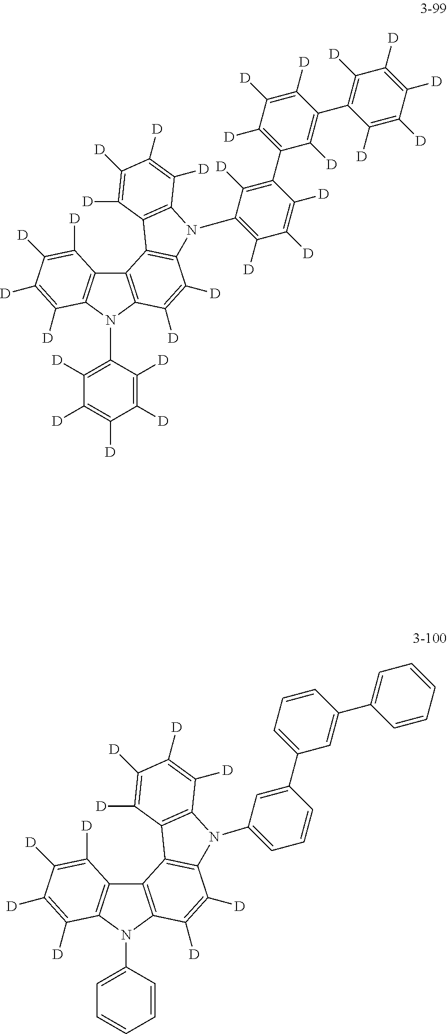 [SMILES: [2H]c1c([2H])c([2H])c(-c2c([2H])c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c3[2H])c2[2H])c([2H])c1[2H] ; [2H]c1c([2H])c([2H])c2c(c1[2H])c1c3c4c([2H])c([2H])c([2H])c([2H])c4n(-c4cccc(-c5cccc(-c6ccccc6)c5)c4)c3c([2H])c([2H])c1n2-c1ccccc1]

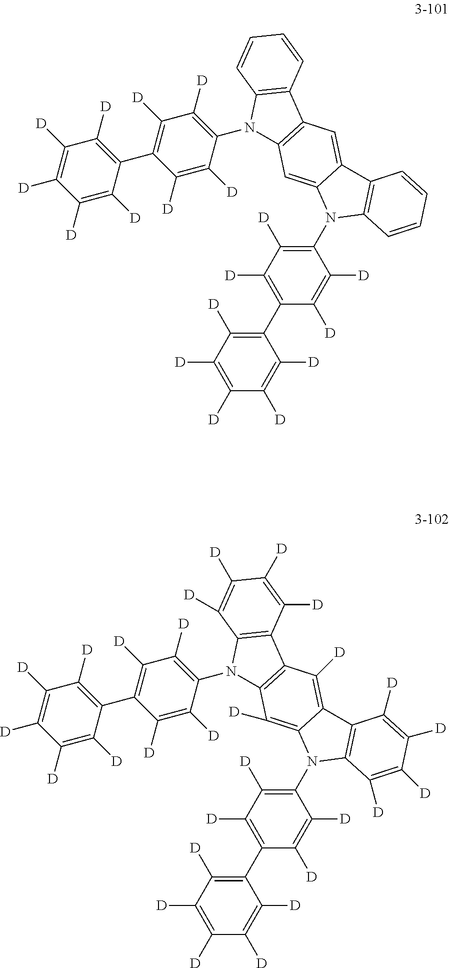 [SMILES: [2H]c1c([2H])c([2H])c(-c2c([2H])c([2H])c(-n3c4ccccc4c4cc5c6ccccc6n(-c6c([2H])c([2H])c(-c7c([2H])c([2H])c([2H])c([2H])c7[2H])c([2H])c6[2H])c5cc43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c43)c([2H])c2[2H])c([2H])c1[2H]]

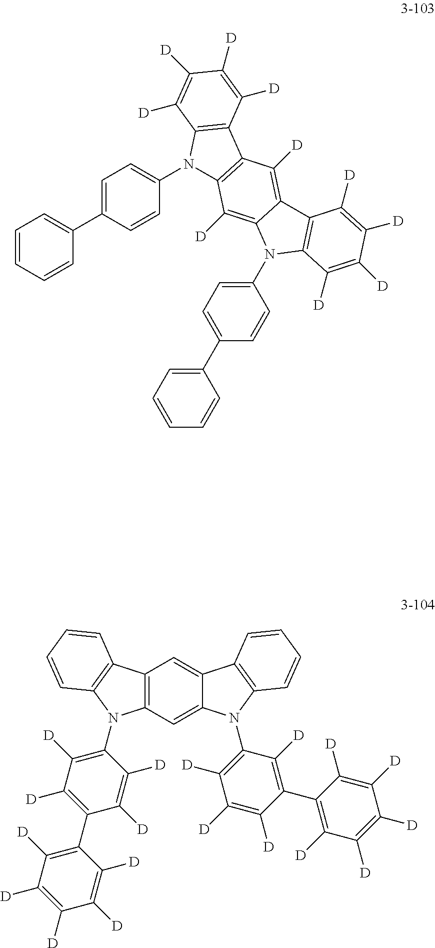 [SMILES: [2H]c1c([2H])c([2H])c2c(c1[2H])c1c([2H])c3c4c([2H])c([2H])c([2H])c([2H])c4n(-c4ccc(-c5ccccc5)cc4)c3c([2H])c1n2-c1ccc(-c2ccccc2)cc1 ; [2H]c1c([2H])c([2H])c(-c2c([2H])c([2H])c(-n3c4ccccc4c4cc5c6ccccc6n(-c6c([2H])c([2H])c([2H])c(-c7c([2H])c([2H])c([2H])c([2H])c7[2H])c6[2H])c5cc43)c([2H])c2[2H])c([2H])c1[2H]]

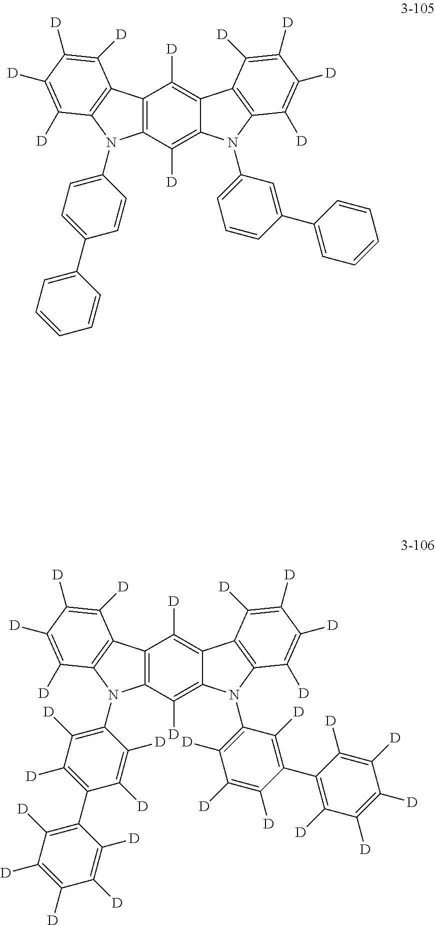 [SMILES: [2H]c1c([2H])c([2H])c2c(c1[2H])c1c([2H])c3c4c([2H])c([2H])c([2H])c([2H])c4n(-c4cccc(-c5ccccc5)c4)c3c([2H])c1n2-c1ccc(-c2ccccc2)cc1 ; [2H]c1c([2H])c([2H])c(-c2c([2H])c([2H])c(-n3c4c([2H])c([2H])c([2H])c([2H])c4c4c([2H])c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c43)c([2H])c2[2H])c([2H])c1[2H]]

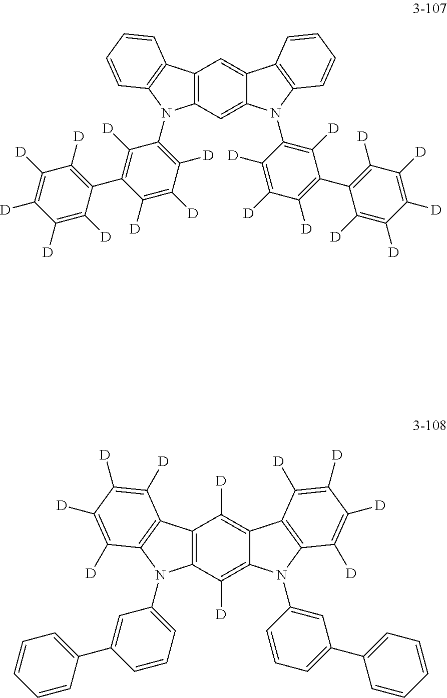 [SMILES: [2H]c1c([2H])c([2H])c(-c2c([2H])c([2H])c([2H])c(-n3c4ccccc4c4cc5c6ccccc6n(-c6c([2H])c([2H])c([2H])c(-c7c([2H])c([2H])c([2H])c([2H])c7[2H])c6[2H])c5cc43)c2[2H])c([2H])c1[2H] ; [2H]c1c([2H])c([2H])c2c(c1[2H])c1c([2H])c3c4c([2H])c([2H])c([2H])c([2H])c4n(-c4cccc(-c5ccccc5)c4)c3c([2H])c1n2-c1cccc(-c2ccccc2)c1]

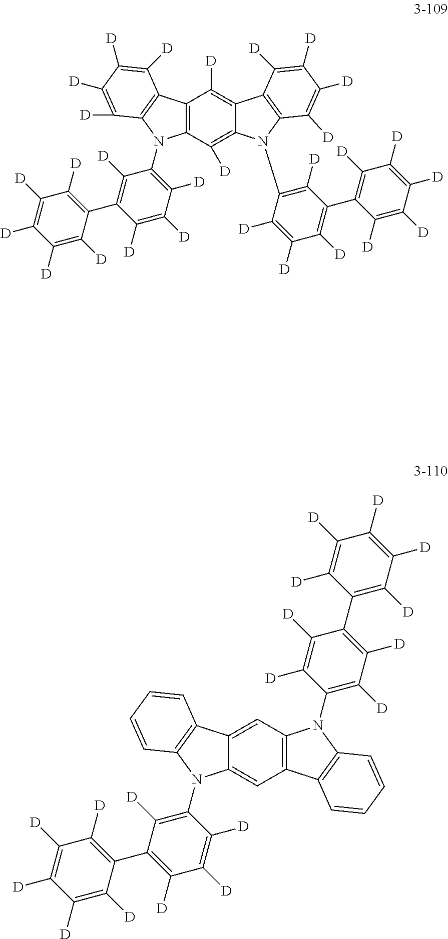 [SMILES: [2H]c1c([2H])c([2H])c(-c2c([2H])c([2H])c([2H])c(-n3c4c([2H])c([2H])c([2H])c([2H])c4c4c([2H])c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c43)c2[2H])c([2H])c1[2H] ; [2H]c1c([2H])c([2H])c(-c2c([2H])c([2H])c(-n3c4ccccc4c4cc5c(cc43)c3ccccc3n5-c3c([2H])c([2H])c([2H])c(-c4c([2H])c([2H])c([2H])c([2H])c4[2H])c3[2H])c([2H])c2[2H])c([2H])c1[2H]]

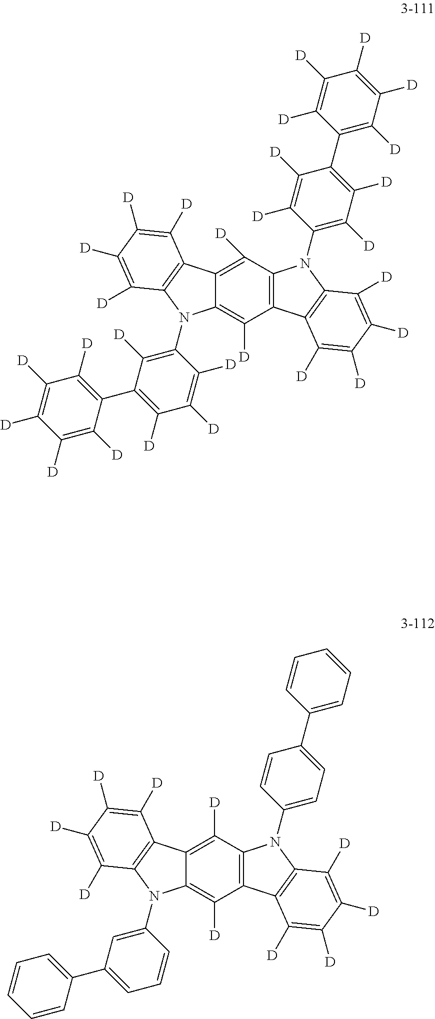 [SMILES: [2H]c1c([2H])c([2H])c(-c2c([2H])c([2H])c(-n3c4c([2H])c([2H])c([2H])c([2H])c4c4c([2H])c5c(c([2H])c43)c3c([2H])c([2H])c([2H])c([2H])c3n5-c3c([2H])c([2H])c([2H])c(-c4c([2H])c([2H])c([2H])c([2H])c4[2H])c3[2H])c([2H])c2[2H])c([2H])c1[2H] ; [2H]c1c([2H])c([2H])c2c(c1[2H])c1c([2H])c3c(c([2H])c1n2-c1ccc(-c2ccccc2)cc1)c1c([2H])c([2H])c([2H])c([2H])c1n3-c1cccc(-c2ccccc2)c1]

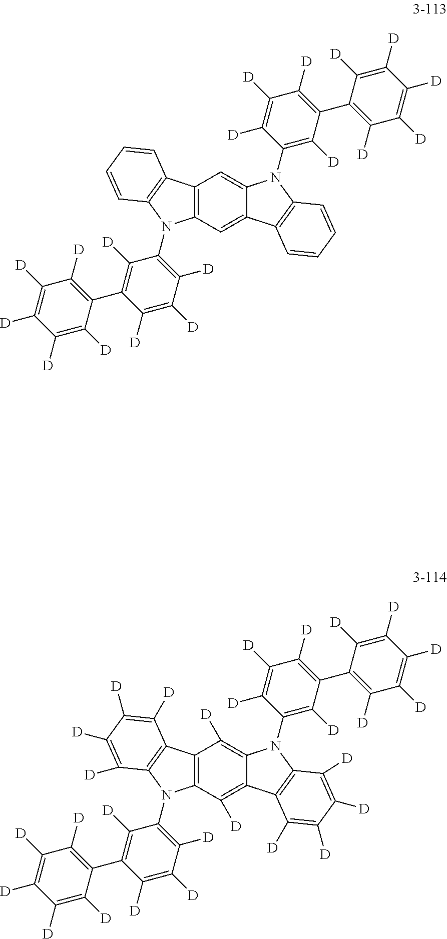 [SMILES: [2H]c1c([2H])c([2H])c(-c2c([2H])c([2H])c([2H])c(-n3c4ccccc4c4cc5c(cc43)c3ccccc3n5-c3c([2H])c([2H])c([2H])c(-c4c([2H])c([2H])c([2H])c([2H])c4[2H])c3[2H])c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c5c(c([2H])c43)c3c([2H])c([2H])c([2H])c([2H])c3n5-c3c([2H])c([2H])c([2H])c(-c4c([2H])c([2H])c([2H])c([2H])c4[2H])c3[2H])c2[2H])c([2H])c1[2H]]

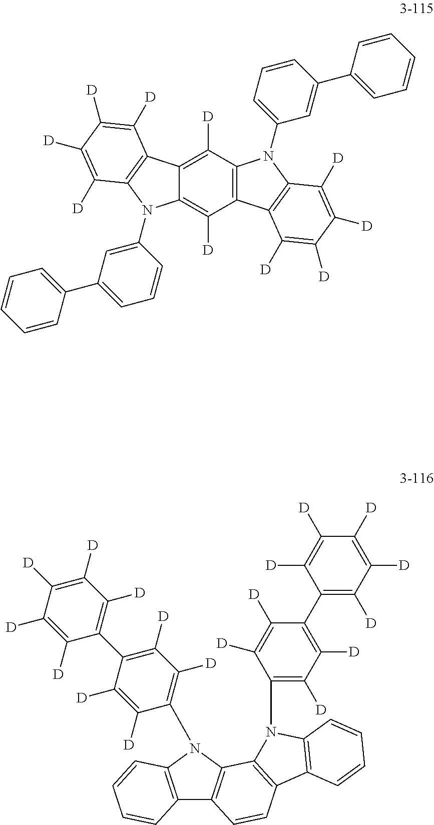 [SMILES: [2H]c1c([2H])c([2H])c2c(c1[2H])c1c([2H])c3c(c([2H])c1n2-c1cccc(-c2ccccc2)c1)c1c([2H])c([2H])c([2H])c([2H])c1n3-c1cccc(-c2ccccc2)c1 ; [2H]c1c([2H])c([2H])c(-c2c([2H])c([2H])c(-n3c4ccccc4c4ccc5c6ccccc6n(-c6c([2H])c([2H])c(-c7c([2H])c([2H])c([2H])c([2H])c7[2H])c([2H])c6[2H])c5c43)c([2H])c2[2H])c([2H])c1[2H]]

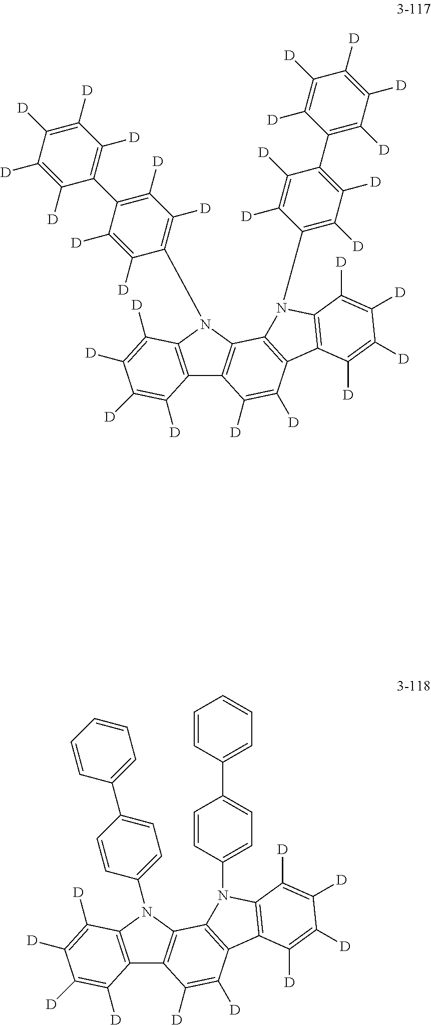 [SMILES: [2H]c1c([2H])c([2H])c(-c2c([2H])c([2H])c(-n3c4c([2H])c([2H])c([2H])c([2H])c4c4c([2H])c([2H])c5c6c([2H])c([2H])c([2H])c([2H])c6n(-c6c([2H])c([2H])c(-c7c([2H])c([2H])c([2H])c([2H])c7[2H])c([2H])c6[2H])c5c43)c([2H])c2[2H])c([2H])c1[2H] ; [2H]c1c([2H])c([2H])c2c(c1[2H])c1c([2H])c([2H])c3c4c([2H])c([2H])c([2H])c([2H])c4n(-c4ccc(-c5ccccc5)cc4)c3c1n2-c1ccc(-c2ccccc2)cc1]

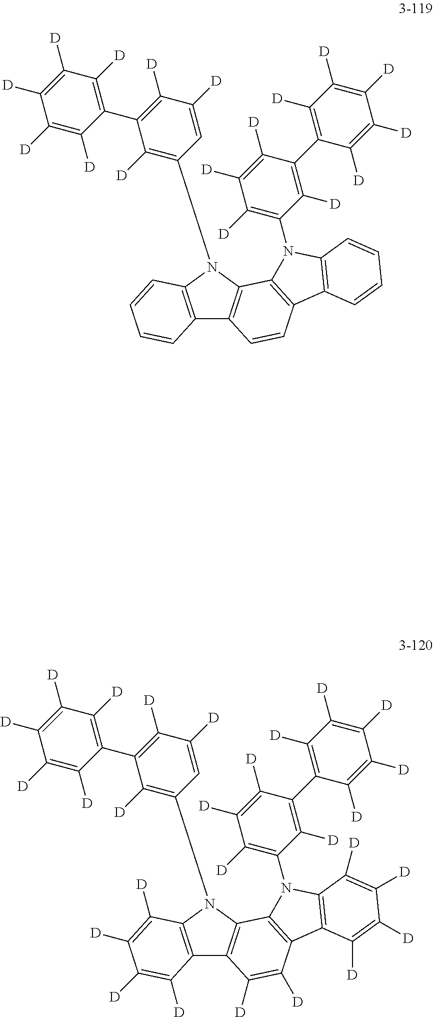 [SMILES: [2H]c1cc(-n2c3ccccc3c3ccc4c5ccccc5n(-c5c([2H])c([2H])c([2H])c(-c6c([2H])c([2H])c([2H])c([2H])c6[2H])c5[2H])c4c32)c([2H])c(-c2c([2H])c([2H])c([2H])c([2H])c2[2H])c1[2H] ; [2H]c1cc(-n2c3c([2H])c([2H])c([2H])c([2H])c3c3c([2H])c([2H])c4c5c([2H])c([2H])c([2H])c([2H])c5n(-c5c([2H])c([2H])c([2H])c(-c6c([2H])c([2H])c([2H])c([2H])c6[2H])c5[2H])c4c32)c([2H])c(-c2c([2H])c([2H])c([2H])c([2H])c2[2H])c1[2H]]

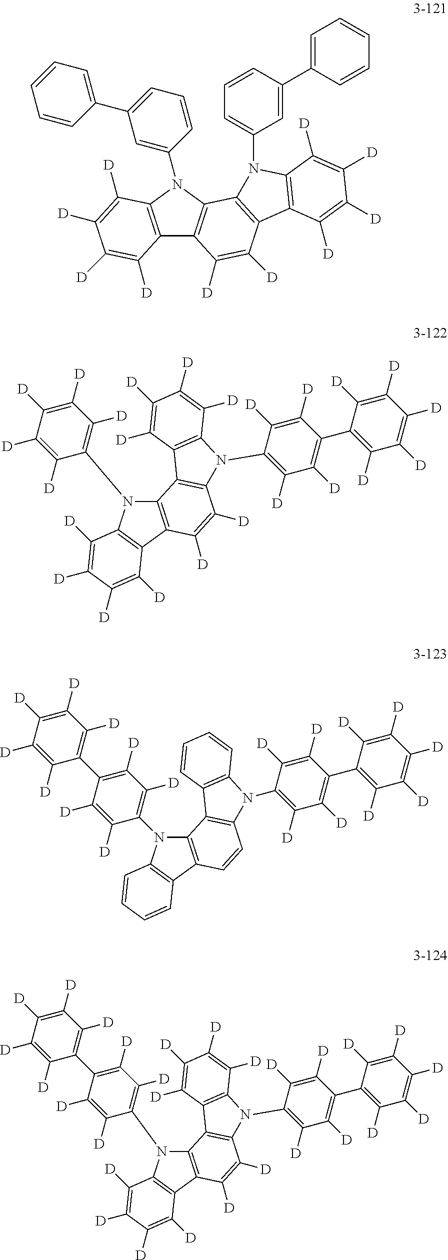

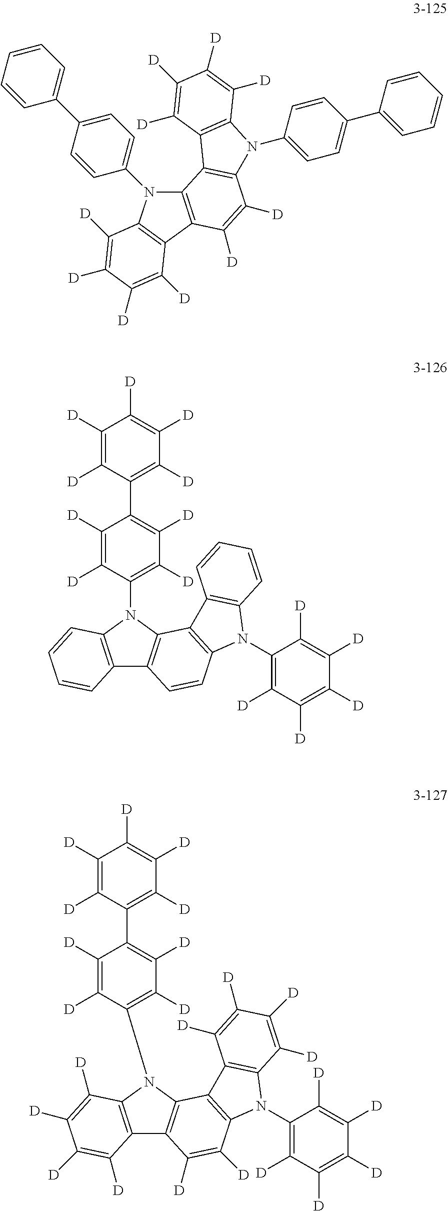 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccc(-c2ccccc2)cc1 ; [2H]c1c([2H])c([2H])c(-c2c([2H])c([2H])c(-n3c4ccccc4c4ccc5c(c6ccccc6n5-c5c([2H])c([2H])c([2H])c([2H])c5[2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)c([2H])c2[2H])c([2H])c1[2H]]

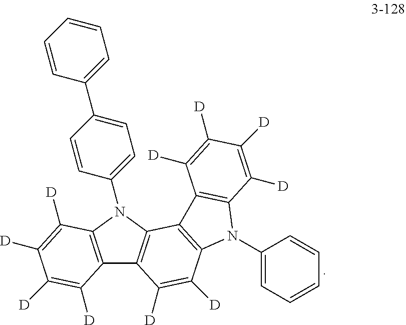 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccccc1]

12. The organic light emitting device of claim 9, wherein one or more layers of the organic material layer comprise a light emitting layer, and the light emitting layer comprises the heterocyclic compound.

13. The organic light emitting device of claim 9, wherein the organic light emitting device further comprises one or two or more layers selected from the group consisting of a light emitting layer, a hole injection layer, a hole transport layer, a hole transport auxiliary layer, an electron injection layer, an electron transport layer, an electron blocking layer, and a hole blocking layer.

14. A composition for an organic material layer of an organic light emitting device, the composition comprising the heterocyclic compound according to claim 1 and a heterocyclic compound represented by the following Chemical Formula 2 or 3: 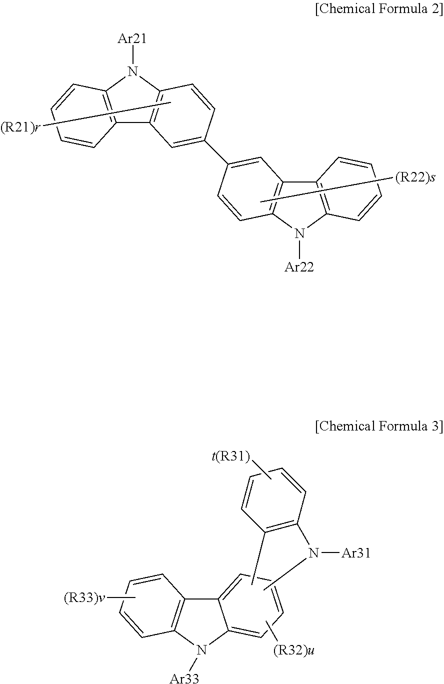
in Chemical Formulae 2 and 3,
R21, R22, and R31 to R33 are the same as or different from each other, and are each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR′R″R′″; —P(═O)R′R″; and an amine group which is unsubstituted or substituted with a substituted or unsubstituted C1 to C60 alkyl group, a substituted or unsubstituted C6 to C60 aryl group or a substituted or unsubstituted C2 to C60 heteroaryl group, or two or more adjacent groups are bonded to each other to form a substituted or unsubstituted C6 to C60 aliphatic or aromatic hydrocarbon ring or a substituted or unsubstituted C2 to C60 aliphatic or aromatic hetero ring,
R′, R″, and R′″ are the same as or different from each other, and are each independently hydrogen; deuterium; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r and s are each an integer from 0 to 7, and when each of r and s is 2 or higher, substituents in the parenthesis are the same as or different from each other,
t and v are each an integer from 0 to 4, and when each of t and v is 2 or higher, substituents in the parenthesis are the same as or different from each other,
u is an integer from 0 to 2, and when u is 2, substituents in the parenthesis are the same as or different from each other, and
Ar21, Ar22, Ar31, and Ar33 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

15. The composition of claim 14, wherein a weight ratio of the heterocyclic compound represented by Chemical Formula 1 to the heterocyclic compound represented by Chemical Formula 2 or 3 is 1:10 to 10:1.
